(19) United States
(12) Patent Application Publication
(10) Pub. No.: US 2009/0069320 A1
(43) Pub. Date: Mar. 12, 2009

(54) Substituted 4-Amino-Quinazoline Compounds with Metabotropic Glutamate Receptor Regulating Activity and Uses Thereof

(75) Inventors: Melanie REICH, Aachen (DE); Stefan Oberboersch, Aachen (DE); Sven Kuehnert, Dueren (DE); Michael Haurand, Aachen (DE); Klaus Schiene, Duesseldorf (DE)

Correspondence Address: CROWELL & MORING LLP;INTELLECTUAL PROPERTY GROUP, P.O. BOX 14300, WASHINGTON, DC, 20044-4300, US

(73) Assignee: Gruenenthal GmbH, Aachen (DE)

(21) Appl. No.: 12/210,365

(22) Filed: Sep. 15, 2008

Related U.S. Application Data
(63) Continuation of application No. PCT/EP2007/002280, filed on Mar. 15, 2007.

(30) Foreign Application Priority Data
Mar. 15, 2006 (DE) 10 2006 012 251.8

(51) Int. Cl. A61K 31/5377; C07D 239/72; A61K 31/517; A61P 29/00; A61P 11/00; A61P 25/00; C07D 413/02

(52) U.S. Cl. 514/234.5; 544/293; 514/266.4; 544/284; 514/266.2; 544/119; 514/266.21

(57) ABSTRACT

Substituted 4-amino-quinazoline compounds corresponding to formula I

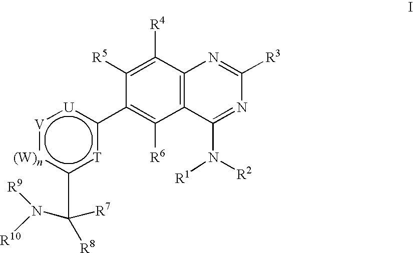

methods for their production, pharmaceutical compositions containing these compounds as active agents, and the uses thereof for treating or inhibiting disorders or disease states.

### CROSS REFERENCE TO RELATED APPLICATIONS

This application is a continuation of international patent application no. PCT/EP2007/02280, filed Mar. 15, 2007, designating the United States of America, and published in German on Sep. 20, 2007 as WO 2007/104560, the entire disclosure of which is incorporated herein by reference. Priority is claimed based on Federal Republic of Germany patent application no. DE 10 2006 012 251.8, filed Mar. 15, 2006.

### BACKGROUND OF THE INVENTION

The present invention relates to substituted 4-amino-quinazoline compounds, methods for their production, pharmaceutical compositions containing these compounds and also their use for producing pharmaceutical compositions.

Pain is one of the basic clinical symptoms. There is a worldwide demand for effective pain therapies. The urgent requirement for a patient-specific and targeted treatment of chronic and non-chronic pain conditions, by which is meant the successful and satisfactory treatment of pain for patients, is also reflected in the large number of scientific papers that have appeared recently in the field of applied analgesics or fundamental research on nociception.

Classic opioids such as morphine, for example, are effective in the therapy of intense to very intense pain, but often result in undesirable side-effects such as e.g. breathing difficulties, vomiting, sedation, constipation or tolerance development. Moreover, they are often poorly effective in the case of neuropathic pain suffered in particular by tumor patients.

### SUMMARY OF THE INVENTION

Therefore, it is an object of the present invention to provide new compounds, which are suitable in particular as pharmaceutical adjuvants in medicaments.

Another object of the invention is to provide new compounds which are especially suitable for the treatment and/or inhibition of pain.

It has now been surprisingly found that the substituted 4-amino-quinazoline compounds corresponding to the following formula I are suitable for mGluR5 receptor regulation and can therefore be used in particular as pharmaceutical adjuvants in medicaments for the inhibition and/or treatment of disorders or diseases associated with these receptors or processes.

Accordingly, the present invention relates to substituted 4-amino-quinazoline compounds corresponding to formula I

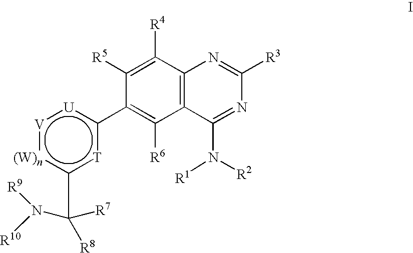

wherein
T represents CR¹¹, N, S or O;
U represents CR¹², N, S or O;
V represents CR¹³, N, S or O;
W represents CR¹⁴ or N;
n represents 0 or 1;
R¹ and R² each independently denote H; —C(═O)—OH; —C(═O)—H; —C(═O)—R¹⁵; —C(═O)—O—R¹⁶; —C(═O)—NH₂; —C(═O)—NH—R¹⁷; —C(═O)—NR¹⁸R¹⁹; —C(═S)—NR¹⁸R¹⁹; —S(═O)—R²⁰; —S(═O)₂—R²¹; unsubstituted or mono- or poly-substituted alkyl, alkenyl or alkinyl; unsubstituted or mono- or poly-substituted heteroalkyl, heteroalkenyl or heteroalkinyl; unsubstituted or mono- or poly-substituted cycloalkyl or cycloalkenyl; unsubstituted or mono- or poly-substituted heterocycloalkyl or heterocycloalkenyl; unsubstituted or mono- or poly-substituted -(alkylene)-cyloalkyl, -(alkenylene)-cycloalkyl, -(alkinylene)-cycloalkyl, -(alkylene)-cycloalkenyl, -(alkenylene)-cycloalkenyl or -(alkinylene)-cycloalkenyl; unsubstituted or mono- or poly-substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or mono- or poly-substituted -(alkylene)-heterocycloalkyl, -(alkenylene)-heterocycloalkyl, -(alkinylene)-heterocycloalkyl, -(alkylene)-heterocycloalkenyl, -(alkenylene)-heterocycloalkenyl or -(alkinylene)-heterocycloalkenyl; unsubstituted or mono- or poly-substituted -(heteroalkylene)-heterocycloalkyl, (heteroalkenylene)-heterocycloalkyl; -(heteroalkylene)-heterocycloalkenyl or -(heteroalkenylene)-heterocycloalkenyl; aryl that can be unsubstituted or substituted with optionally 1, 2, 3, 4 or 5 substituents independently selected from the group consisting of F, Cl, Br, I, —CN, —NO₂, —OH, —SH, —NH₂, —C(═O)—OH, —C₁₋₅-alkyl, —(CH₂)—O—C₁₋₅-alkyl, —C₂₋₅-alkenyl, —C₂₋₅-alkinyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, —S(═O)₂—C₁₋₅-alkyl, —S(═O)—C₁₋₅-alkyl, —NH—C₁₋₅-alkyl, N(C₁₋₅-alkyl)₂, —C(═O)—O—C₁₋₅-alkyl, —C(═O)—H, —C(═O)—C₁₋₅-alkyl, —CH₂—O—C(═O)-phenyl, —O—C(═O)-phenyl, —NH—S(═O)₂—C₁₋₅-alkyl, —NH—C(═O)—C₁₋₅-alkyl, —C(═O)—NH₂, —C(═O)—NH—C₁₋₅-alkyl, —C(═O)—N(C₁₋₅-alkyl)₂, cyclopropyl, cyclobutyl, cyclopentyl, cyclohexyl, piperazinyl, pyrrolidinyl, piperidinyl, pyrazolyl, phenyl, furyl (furanyl), thiazolyl, thiadiazolyl, thiophenyl (thienyl), benzyl and phenethyl, wherein the cyclic substituents or the cyclic residues of these substituents can themselves be substituted with optionally 1, 2, 3, 4 or 5 substituents independently selected from the group consisting of F, Cl, Br, I, —CN, —NO₂, —OH, —SH, —NH₂, —C(═O)—OH, —C₁₋₅-alkyl, —(CH₂)—O—C₁₋₅-alkyl, —C₂₋₅-alkenyl, —C₂₋₅-alkinyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂, —S—CH₂F; unsubstituted or mono- or poly-substituted heteroaryl; unsubstituted or mono- or poly-substituted -(alkylene)-aryl, -(alkenylene)-aryl, (alkinylene)-aryl, (heteroalkylene)-aryl or -(heteroalkenylene)-aryl; or unsubstituted or mono- or poly-substituted -(alkylene)-heteroaryl, -(alkenylene)-heteroaryl, -(alkinylene)-heteroaryl, -(heteroalkylene)-heteroaryl or -(heteroalkenylene)-heteroaryl; or
R¹ and R² together with the nitrogen atom to which they are bound represent unsubstituted or mono- or poly-substituted heterocycloalkyl or heterocycloalkenyl;
R³ represents H; F; Cl; Br; I; —NO₂; —CN, —NH₂, —OH; —SH; —C(═O)—OH; —C(═O)—H; —C(═O)—R¹⁵; —C(═O)—O—R¹⁶; —C(═O)—NH₂; —C(═O)—NH—R¹⁷; —C(═S)—NH—R¹⁷; —C(═O)—NR¹⁸R¹⁹; —C(═S)—NR¹⁸R¹⁹; —S(═O)—R²⁰; —S(═O)₂—R²¹; —NH—R²²; —NR²³R²⁴; —O—R²⁵; —S—R²⁶; —O—C(═O)—R²⁷; —NH—C(═O)—H; —NH—C(═O)—R²⁸; —NR²⁹—C(═O)—R³⁰; —NH—C(═O)—NH—R³¹; —NH—C(═S)—NH—R³²; —NH—S(═O)₂—R³³; —NR³⁴—S(═O)₂—R³⁵; unsubstituted or mono- or poly-substituted alkyl, alkenyl or alkinyl; unsubstituted or mono- or poly-substituted heteroalkyl, heteroalkenyl or heteroalkinyl; unsubstituted or mono- or poly-substituted cycloalkyl or cycloalkenyl; unsubstituted or mono- or poly-substituted heterocycloalkyl or heterocycloalkenyl; unsubstituted or mono- or poly-substituted aryl; unsubstituted or mono- or poly-substituted heteroaryl; unsubstituted or mono- or poly-substituted -(alkylene)-aryl, -(alkenylene)-aryl, (alkinylene)-aryl, (heteroalkylene)-aryl or -(heteroalkenylene)-aryl; or unsubstituted or mono- or poly-substituted -(alkylene)-heteroaryl, -(alkenylene)-heteroaryl, -(alkinylene)-heteroaryl, -(heteroalkylene)-heteroaryl or -(heteroalkenylene)-heteroaryl;
R⁴, R⁵ and R⁶ each independently represent H; F; Cl; Br; I; —NO₂; —CN, —NH₂, —OH; —SH; —C(═O)—OH; —C(═O)—H; —C(═O)—R¹⁵; —C(═O)—O—R¹⁶; —C(═O)—NH₂; —C(═O)—NH—R¹⁷; —C(═S)—NH—R¹⁷; —C(═O)—NR¹⁸R¹⁹; —C(═S)—NR¹⁸R¹⁹; —S(═O)—R²⁰; —S(═O)₂—R²¹; —NH—R²²; NR²³R²⁴; —O—R²⁵; —S—R²⁶; —O—C(═O)—R²⁷; —NH—C(═O)—H; —NH—C(═O)—R²⁸; —NR²⁹—C(═O)—R³⁰; —NH—C(═O)—NH—R³¹; —NH—C(═S)—NH—R³²; —NH—S(═O)₂—R³³; —NR³⁴—S(═O)₂—R³⁵; unsubstituted or mono- or poly-substituted alkyl, alkenyl or alkinyl; unsubstituted or mono- or poly-substituted heteroalkyl, heteroalkenyl or heteroalkinyl; unsubstituted or mono- or poly-substituted cycloalkyl or cycloalkenyl; unsubstituted or mono- or poly-substituted heterocycloalkyl or heterocycloalkenyl; unsubstituted or mono- or poly-substituted aryl; unsubstituted or mono- or poly-substituted heteroaryl; unsubstituted or mono- or poly-substituted -(alkylene)-aryl, -(alkenylene)-aryl, (alkinylene)-aryl, (heteroalkylene)-aryl or -(heteroalkenylene)-aryl; or unsubstituted or mono- or poly-substituted -(alkylene)-heteroaryl, -(alkenylene)-heteroaryl, -(alkinylene)-heteroaryl, -(heteroalkylene)-heteroaryl or -(heteroalkenylene)-heteroaryl;
R⁷ and R⁸ each independently represent H; F; Cl; Br; I; —NO₂; —CN, —NH₂, —OH; —SH; —C(═O)—OH; —C(═O)—H; —C(═O)—R¹⁵; —C(═O)—O—R¹⁶; —C(═O)—NH₂; —C(═O)—NH—R¹⁷; —C(═S)—NH—R¹⁷; —C(═O)—NR¹⁸R¹⁹; —C(═S)—NR¹⁸R¹⁹; —S(═O)—R²⁰; —S(═O)₂—R²¹; —NH—R²²; —NR²³R²⁴; —O—R²⁵; —S—R²⁶; —O—C(═O)—R²⁷; —NH—C(═O)—H; —NH—C(═O)—R²⁸; —NR²⁹—C(═O)—R³⁰; —NH—C(═O)—NH—R³¹; —NH—C(═S)—NH—R³²; —NH—S(═O)₂—R³³; —NR³⁴—S(═O)₂—R³⁵; unsubstituted or mono- or poly-substituted alkyl, alkenyl or alkinyl; unsubstituted or mono- or poly-substituted heteroalkyl, heteroalkenyl or heteroalkinyl; unsubstituted or mono- or poly-substituted cycloalkyl or cycloalkenyl; unsubstituted or mono- or poly-substituted heterocycloalkyl or heterocycloalkenyl; unsubstituted or mono- or poly-substituted aryl; unsubstituted or mono- or poly-substituted heteroaryl; unsubstituted or mono- or poly-substituted -(alkylene)-aryl, -(alkenylene)-aryl, (alkinylene)-aryl, (heteroalkylene)-aryl or -(heteroalkenylene)-aryl; or unsubstituted or mono- or poly-substituted -(alkylene)-heteroaryl, -(alkenylene)-heteroaryl, -(alkinylene)-heteroaryl, -(heteroalkylene)-heteroaryl or -(heteroalkenylene)-heteroaryl;
R⁹ represents H, —C(═O)—OH; —C(═O)—H; —C(═O)—R¹⁵; —C(═O)—O—R¹⁶; —C(═O)—NH₂; —C(═O)—NH—R¹⁷; —C(═S)—NH—R¹⁷; —C(═O)—NR¹⁸R¹⁹; —C(═S)—NR¹⁸R¹⁹; —S(═O)—R²⁰; —S(═O)₂—R²¹; unsubstituted or mono- or poly-substituted alkyl, alkenyl or alkinyl; unsubstituted or mono- or poly-substituted heteroalkyl, heteroalkenyl or heteroalkinyl; unsubstituted or mono- or poly-substituted cycloalkyl or cycloalkenyl; unsubstituted or mono- or poly-substituted heterocycloalkyl or heterocycloalkenyl; unsubstituted or mono- or poly-substituted -(alkylene)-cyloalkyl, -(alkenylene)-cycloalkyl, -(alkinylene)-cycloalkyl, -(alkylene)-cycloalkenyl, -(alkenylene)-cycloalkenyl or -(alkinylene)-cycloalkenyl; unsubstituted or mono- or poly-substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or mono- or poly-substituted -(alkylene)-heterocycloalkyl, -(alkenylene)-heterocycloalkyl, -(alkinylene)-heterocycloalkyl, -(alkylene)-heterocycloalkenyl, -(alkenylene)-heterocycloalkenyl or -(alkinylene)-heterocycloalkenyl; unsubstituted or mono- or poly-substituted -(heteroalkylene)-heterocycloalkyl, (heteroalkenylene)-heterocycloalkyl-(heteroalkylene)-heterocycloalkenyl or -(heteroalkenylene)-heterocycloalkenyl; unsubstituted or mono- or poly-substituted aryl; unsubstituted or mono- or poly-substituted heteroaryl; unsubstituted or mono- or poly-substituted -(alkylene)-aryl, -(alkenylene)-aryl, (alkinylene)-aryl, (heteroalkylene)-aryl or -(heteroalkenylene)-aryl; or unsubstituted or mono- or poly-substituted -(alkylene)-heteroaryl, -(alkenylene)-heteroaryl, -(alkinylene)-heteroaryl, -(heteroalkylene)-heteroaryl or -(heteroalkenylene)-heteroaryl;
R¹⁰ represents —C(═O)—OH; —C(═O)—H; —C(═O)—R¹⁵; —C(═O)—O—R¹⁶; —C(═O)—NH₂; —C(═O)—NH—R¹⁷; —C(═S)—NH—R¹⁷; —C(═O)—NR¹⁸R¹⁹; —C(═S)—NR¹⁸R¹⁹; —S(═O)—R²⁰; —S(═O)₂—R²¹; alkyl, alkenyl or alkinyl, which is respectively substituted with 1, 2, 3, 4 or 5 substituents independently selected from the group consisting of F, Cl, Br, I, —NO₂, —CN, —SH, —NH₂, —N(C₁₋₅)-alkyl)₂, —N(C₁₋₅-alkyl)(phenyl), —N(C₁₋₅-alkyl)(CH₂-phenyl), —N(C₁₋₅-alkyl)(CH₂—CH₂-phenyl), NH—C(═O)—O—C₁₋₅-alkyl, —C(═O)—H, —C(═O)—C₁₋₅-alkyl, —C(═O)-phenyl, —C(═S)—C₁₋₅-alkyl, —C(═S)-phenyl, —C(═O)—OH, —C(═O)—O—C₁₋₅-alkyl, —C(═O)—O-phenyl, C(═O)—NH₂, —C(═O)—NH—C₁₋₅-alkyl, —C(═O)—N(C₁₋₅-alkyl)₂, —S(═O)—C₁₋₅-alkyl, —S(═O)-phenyl, —S(═O)₂-phenyl, —S(═O)₂—NH₂ and —SO₃H, wherein the phenyl residues can be substituted with 1, 2, 3, 4 or 5 substituents independently selected from the group consisting of F, Cl, Br, I, —CN, CF₃, —OH, —NH₂, O—CF₃, —SH, —O—CH₃, —O—C₂H₅, —O—C₃H₇, methyl, ethyl, n-propyl, isopropyl, n-butyl, 2-butyl, isobutyl and tert-butyl; unsubstituted or mono- or poly-substituted heteroalkenyl or heteroalkinyl; unsubstituted or mono- or poly-substituted cycloalkyl or cycloalkenyl; unsubstituted or mono- or poly-substituted heterocycloalkyl or heterocycloalkenyl; unsubstituted or mono- or poly-substituted -(alkylene)-cyloalkyl, -(alkenylene)-cycloalkyl, -(alkinylene)-cycloalkyl, -(alkylene)-cycloalkenyl, -(alkenylene)-cycloalkenyl or -(alkinylene)-cycloalkenyl; unsubstituted or mono- or poly-substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or mono- or poly-substituted -(alkylene)-heterocycloalkyl, -(alkenylene)-heterocycloalkyl, -(alkinylene)-heterocycloalkyl, -(alkylene)-heterocycloalkenyl, -(alkenylene)-heterocycloalkenyl or -(alkinylene)-heterocycloalkenyl; unsubstituted or mono- or poly-substituted -(heteroalkylene)-heterocycloalkyl, (heteroalkenylene)-heterocycloalkyl-(heteroalkylene)-heterocycloalkenyl or -(heteroalkenylene)-heterocycloalkenyl; unsubstituted or mono- or poly-substituted aryl; unsubstituted or mono- or poly-substituted heteroaryl; unsubstituted or mono- or poly-substituted -(alkylene)-aryl, -(alkenylene)-aryl, (alkinylene)-aryl, (heteroalkylene)-aryl or -(heteroalkenylene)-aryl; or unsubstituted or mono- or poly-substituted -(alkylene)-heteroaryl, -(alkenylene)-heteroaryl, -(alkinylene)-heteroaryl, -(heteroalkylene)-heteroaryl or -(heteroalkenylene)-heteroaryl; or
R⁹ and R¹⁰ together with the nitrogen atom linking them form a residue selected from the group consisting of imidazolidinyl, azepanyl, pyrrolyl, diazepanyl, isothioazolidinyl, (2,3)-dihydro-1H-isoindolyl, indolinyl, octahydro-1H-isoindolyl, (2,3)-dihydropyrrolyl, (2,5)-dihydropyrrolyl, 8-azaspiro[4.5]decyl, 8-azaspiro[4.5]decane-7,9-dionyl, isoindoline-1,3-dionyl, pyrrolidine-2,5-dionyl, morpholine-3,5-dionyl, 1H-pyrrole-2,5-dionyl, piperidine-2,6-dionyl, hexahydro-2H-isoindoline-1,3-dionyl, pyrrolidine-2-onyl and 1,1-dioxo-1λ⁶-isothiazolidinyl, which can be respectively unsubstituted or substituted with optionally 1, 2, 3, 4 or 5 substituents independently selected from the group consisting of F, Cl, Br, I, —CN, CF₃, —OH, —NH₂, O—CF₃, —SH, —O—C₁₋₅-alkyl, —O-phenyl, —O—CH₂-phenyl, —(CH₂)—O—C₁₋₅-alkyl, —S—C₁₋₅-alkyl, —S-phenyl, —S—CH₂-phenyl, —C₁₋₅-alkyl, —C₂₋₅-alkenyl, —C₂₋₅-alkinyl, —C≡C—Si(CH₃)₃, —C≡C—Si(C₂H₅)₃, —C(═O)—O—C₁₋₅-alkyl, —C(═O)—CF₃, —S(═O)₂—C₁₋₅-alkyl, —S(═O)—C₁₋₅-alkyl, —S(═O)₂-phenyl, thioxo (═S), —N(C₁₋₅-alkyl)₂, —N(H)(C₁₋₅-alkyl), —NO₂, —S—CF₃, —C(═O)—OH, —NH—S(═O)₂—C₁₋₅-alkyl, —NH—C(═O)—C₁₋₅-alkyl, —NH—C(═O)—CF₃, —C(═O)—H, —C(═O)—C₁₋₅-alkyl, —C(═O)—NH₂, —C(═O)—N(C₁₋₅-alkyl)₂, —C(═O)—N(H)(C₁₋₅-alkyl) and phenyl, wherein the phenyl residues can be respectively unsubstituted or substituted with 1, 2, 3, 4 or 5 substituents independently selected from the group consisting of F, Cl, Br, I, —CN, —CF₃, —OH, —NH₂, —O—CF₃, —SH, —O—C₁₋₅-alkyl, —O-phenyl, —O—CH₂-phenyl, —(CH₂)—O—C₁₋₅-alkyl, —S—C₁₋₅-alkyl, —S-phenyl, —S—CH₂-phenyl, —C₁₋₅-alkyl, —C₂₋₅-alkenyl, —C₂₋₅-alkinyl, —C≡C—Si(CH₃)₃, —C≡C—Si(C₂H₅)₃, —C(═O)—O—C₁₋₅-alkyl and —C(═O)—CF₃, wherein the abovementioned phenyl residues can be substituted with 1, 2, 3, 4 or 5 substituents independently selected from the group consisting of F, Cl, Br, I, —CN, —CF₃, —OH, —NH₂, —O—CF₃, —SH, —O—CH₃, —O—C₂H₅, —O—C₃H₇, methyl, ethyl, n-propyl, isopropyl, n-butyl, 2-butyl, isobutyl and tert-butyl; and
if R¹ represents a residue selected from the group consisting of unsubstituted C₁₋₁₂-alkyl, cyclopropyl and cyclobutyl, preferably for a residue selected from the group consisting of methyl, ethyl, n-propyl, isopropyl, n-butyl, isobutyl, 2-butyl, tert-butyl and cyclopropyl, then R¹⁰ may additionally represent H, unsubstituted alkyl or unsubstituted or mono- or poly-substituted heteroalkyl;
R¹¹, R¹², R¹³ and R¹⁴ each independently represent H; F; Cl; Br; I; —NO₂; —CN, —NH₂, —OH; —SH; —C(═O)—OH; —C(═O)—H; —C(═O)—R¹⁵; —C(═O)—O—R¹⁶; —C(═O)—NH₂; —C(═O)—NH—R¹⁷; —C(═S)—NH—R¹⁷; —C(═O)—NR¹⁸R¹⁹; —C(═S)—NR¹⁸R¹⁹; —S(═O)—R²⁰; —S(═O)₂—R²¹; —NH—R²²; —NR²³R²⁴; —O—R²⁵; —S—R²⁶; —O—C(═O)—R²⁷; —NH—C(═O)—H; —NH—C(═O)—R²⁸; —NR²⁹—C(═O)—R³⁰; —NH—C(═O)—NH—R³¹; —NH—C(═S)—NH—R³²; —NH—S(═O)₂—R³³; —NR³⁴—S(═O)₂—R³⁵; unsubstituted or mono- or poly-substituted alkyl, alkenyl or alkinyl; unsubstituted or mono- or poly-substituted heteroalkyl, heteroalkenyl or heteroalkinyl; unsubstituted or mono- or poly-substituted cycloalkyl or cycloalkenyl; unsubstituted or mono- or poly-substituted heterocycloalkyl or heterocycloalkenyl; unsubstituted or mono- or poly-substituted aryl; unsubstituted or mono- or poly-substituted heteroaryl; unsubstituted or mono- or poly-substituted -(alkylene)-aryl, -(alkenylene)-aryl, (alkinylene)-aryl, (heteroalkylene)-aryl or -(heteroalkenylene)-aryl; or unsubstituted or mono- or poly-substituted -(alkylene)-heteroaryl, -(alkenylene)-heteroaryl, -(alkinylene)-heteroaryl, -(heteroalkylene)-heteroaryl or -(heteroalkenylene)-heteroaryl;
R¹⁵ represents —C(═O)—O—R¹⁶; unsubstituted or mono- or poly-substituted alkyl, alkenyl or alkinyl; unsubstituted or mono- or poly-substituted heteroalkyl, heteroalkenyl or heteroalkinyl; unsubstituted or mono- or poly-substituted cycloalkyl or cycloalkenyl; unsubstituted or mono- or poly-substituted heterocycloalkyl or heterocycloalkenyl; unsubstituted or mono- or poly-substituted -(alkylene)-cyloalkyl, -(alkenylene)-cycloalkyl, or -(alkinylene)-cycloalkyl, -(alkylene)-cycloalkenyl, -(alkenylene)-cycloalkenyl or -(alkinylene)-cycloalkenyl; unsubstituted or mono- or poly-substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or mono- or poly-substituted -(alkylene)-heterocycloalkyl, -(alkenylene)-heterocycloalkyl, -(alkinylene)-heterocycloalkyl, -(alkylene)-heterocycloalkenyl, -(alkenylene)-heterocycloalkenyl or -(alkinylene)-heterocycloalkenyl; unsubstituted or mono- or poly-substituted -(heteroalkylene)-heterocycloalkyl, (heteroalkenylene)-heterocycloalkyl; -(heteroalkylene)-heterocycloalkenyl or -(heteroalkenylene)-heterocycloalkenyl; unsubstituted or mono- or poly-substituted aryl; unsubstituted or mono- or poly-substituted heteroaryl; unsubstituted or mono- or poly-substituted -(alkylene)-aryl, -(alkenylene)-aryl, (alkinylene)-aryl, (heteroalkylene)-aryl or -(heteroalkenylene)-aryl; or unsubstituted or mono- or poly-substituted -(alkylene)-heteroaryl, -(alkenylene)-heteroaryl, -(alkinylene)-heteroaryl, -(heteroalkylene)-heteroaryl or -(heteroalkenylene)-heteroaryl; and
R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², R³³, R³⁴ and R³⁵ each independently represent unsubstituted or mono- or poly-substituted alkyl, alkenyl or alkinyl; unsubstituted or mono- or poly-substituted heteroalkyl, heteroalkenyl or heteroalkinyl; unsubstituted or mono- or poly-substituted cycloalkyl or cycloalkenyl; unsubstituted or mono- or poly-substituted heterocycloalkyl or heterocycloalkenyl; unsubstituted or mono- or poly-substituted -(alkylene)-cyloalkyl, -(alkenylene)-cycloalkyl, or -(alkinylene)-cycloalkyl, -(alkylene)-cycloalkenyl, -(alkenylene)-cycloalkenyl or -(alkinylene)-cycloalkenyl; unsubstituted or mono- or poly-substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or mono- or poly-substituted -(alkylene)-heterocycloalkyl, -(alkenylene)-heterocycloalkyl, -(alkinylene)-heterocycloalkyl, -(alkylene)-heterocycloalkenyl, -(alkenylene)-heterocycloalkenyl or -(alkinylene)-heterocycloalkenyl; unsubstituted or mono- or poly-substituted -(heteroalkylene)-heterocycloalkyl, (heteroalkenylene)-heterocycloalkyl; -(heteroalkylene)-heterocycloalkenyl or -(heteroalkenylene)-heterocycloalkenyl; unsubstituted or mono- or poly-substituted aryl; unsubstituted or mono- or poly-substituted heteroaryl; unsubstituted or mono- or poly-substituted -(alkylene)-aryl, -(alkenylene)-aryl, (alkinylene)-aryl, (heteroalkylene)-aryl or -(heteroalkenylene)-aryl; or unsubstituted or mono- or poly-substituted -(alkylene)-heteroaryl, -(alkenylene)-heteroaryl, -(alkinylene)-heteroaryl, -(heteroalkylene)-heteroaryl or -(heteroalkenylene)-heteroaryl;
   in each case optionally in the form of one of their pure stereoisomers, in particular enantiomers or diastereomers, their racemates, or in the form of a mixture of stereoisomers, in particular the enantiomers and/or diastereomers, in any mixture ratio, or respectively in the form of corresponding salts or respectively in the form of corresponding solvates.

As used herein, the term “alkyl” refers to acyclic saturated hydrocarbon residues, which can be branched or straight-chain as well as unsubstituted or mono- or poly-substituted with, as in the case of C₁₋₁₂-alkyl, 1 to 12 (i.e. 1, 2, 3, 4, 5, 6, 7, 8, 9, 10, 11 or 12) C atoms, or as in the case of C₁₋₆-alkyl, 1 to 6 (i.e. 1, 2, 3, 4, 5 or 6) C atoms. Where one or more of the substituents represent an alkyl residue or have an alkyl residue, which is mono- or multiply-substituted, this can preferably be substituted with optionally 1, 2, 3, 4 or 5, particularly preferred with 1, 2 or 3, substituents independently selected from the group consisting of F, Cl, Br, I, —NO₂, —CN, —OH, —SH, —NH₂, —N(C₁₋₅-alkyl)₂, —N(C₁₋₅-alkyl)(phenyl), —N(C₁₋₅-alkyl)(CH₂-phenyl), —N(C₁₋₅-alkyl)(CH₂—CH₂-phenyl), —NH—C(═O)—O—C₁₋₅-alkyl, —C(═O)—H, —C(═O)—C₁₋₅-alkyl, —C(═O)-phenyl, —C(═S)—C₁₋₅-alkyl, —C(═S)-phenyl, —C(═O)—OH, —C(═O)—O—C₁₋₅-alkyl, —C(═O)—O-phenyl, —C(═O)—NH₂, —C(═O)—NH—C₁₋₅-alkyl, —C(═O)—N(C₁₋₅-alkyl)₂, —S(═O)—C₁₋₅-alkyl, —S(═O)-phenyl, —S(═O)₂—C₁₋₅-alkyl, —S(═O)₂-phenyl, —S(═O)₂—NH₂ and —SO₃H, wherein the abovementioned C₁₋₅-alkyl residues can respectively be linear or branched and the abovementioned phenyl residues can preferably be substituted with 1, 2, 3, 4 or 5 substituents independently selected from the group consisting of F, Cl, Br, I, —CN, —CF₃, —OH, —NH₂, —O—CF₃, —SH, —O—CH₃, —O—C₂H₅, —O—C₃H₇, methyl, ethyl, n-propyl, isopropyl, n-butyl, 2-butyl, isobutyl and tert-butyl. Particularly preferred substituents can be independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃.

Suitable C₁₋₁₂-alkyl residues, which can be unsubstituted or mono- or multiply-substituted, include, for example, methyl, ethyl, n-propyl, isopropyl, n-butyl, isobutyl, 2-butyl, tert-butyl, n-pentyl, 2-pentyl, 3-pentyl, iso-pentyl, neo-pentyl, n-hexyl, 2-hexyl, 3-hexyl, n-heptyl, n-octyl, —C(H)(C₂H₅)₂, —C(H)(n-C₃H₇)₂ and —CH₂—CH₂—C(H)(CH₃)—(CH₂)₃—CH₃. Suitable C₁₋₆-alkyl residues are, for example, methyl, ethyl, n-propyl, isopropyl, n-butyl, isobutyl, 2-butyl, tert-butyl, n-pentyl, 2-pentyl, 3-pentyl, iso-pentyl, neo-pentyl, n-hexyl, 2-hexyl and 3-hexyl.

Multiply-substituted alkyl residues are understood to be those alkyl residues, which are substituted either on different C atoms or on the same C atoms multiple times, preferably twice or three times, e.g. three times on the same C atom as in the case of —CF₃ or at different points such as in the case of —(CHCl)—(CH₂F). Multiple substitution can occur with the same or with different substituents. Suitable substituted alkyl residues include, for example, —CF₃, —CF₂H, —CFH₂, —CH₂Cl, —(CH₂)—OH, —(CH₂)—NH₂, —(CH₂)—CN, —(CH₂)—(CF₃), —(CH₂)—(CHF₂), —(CH₂)—(CH₂F), —(CH₂)—(CH₂Cl), —(CH₂)—(CH₂)—OH, —(CH₂)—(CH₂)—NH₂, —(CH₂)—(CH₂)—CN, —(CF₂)—(CF₃), —(CH₂)—(CH₂)—(CF₃), —(CH₂)—(CH₂)—(CH₂)—OH, —(CH₂)—N(CH₃)₂, —(CH₂)—(CH₂)—(CH₂)—Cl, —(CH₂)—(CH₂)—(CH₂)—(CH₂)—Cl, —(CH₂)—C(═O)—OH, —(CH₂)—(CH₂)—C(═O)—OH, —(CH₂)—C(═O)—O—CH₃ and —(CH₂)—(CH₂)—NH—C(═O)—O—C(CH₃)₃.

As used herein, the term “alkenyl” refers to acyclic unsaturated hydrocarbon residues, which can be branched or straight-chain as well as unsubstituted or mono- or poly-substituted and have at least one double bond, preferably 1, 2 or 3 double bonds with, as in the case of C₂₋₁₂-alkenyl, 2 to 12 (i.e. 2, 3, 4, 5, 6, 7, 8, 9, 10, 11 or 12) C atoms, or, as in the case of C₂₋₆-alkenyl, 2 to 6 (i.e. 2, 3, 4, 5 or 6) C atoms. Where one or more of the substituents denote an alkenyl residue or contain an alkenyl residue, which is mono- or multiply-substituted, this can preferably be substituted with optionally 1, 2, 3, 4 or 5, particularly preferably with 1, 2 or 3, substituents independently selected from the group consisting of F, Cl, Br, I, —NO₂, —CN, —OH, —SH, —NH₂, —N(C₁₋₅-alkyl)₂, —N(C₁₋₅-alkyl)(phenyl), —N(C₁₋₅-alkyl)(CH₂-phenyl), —N(C₁₋₅-alkyl)(CH₂—CH₂-phenyl), —NH—C(═O)—O—C₁₋₅-alkyl, —C(═O)—H, —C(═O)—C₁₋₅-alkyl, —C(═O)-phenyl, —C(═S)—C₁₋₅-alkyl, —C(═S)-phenyl, —C(═O)—OH, —C(═O)—O—C₁₋₅-alkyl, —C(═O)—O-phenyl, —C(═O)—NH₂, —C(═O)—NH—C₁₋₅-alkyl, —C(═O)—N(C₁₋₅-alkyl)₂, —S(═O)—C₁₋₅-alkyl, —S(═O)-phenyl, —S(═O)₂—C₁₋₅-alkyl, —S(═O)₂-phenyl, —S(═O)₂—NH₂ and —SO₃H, wherein the abovementioned C₁₋₅-alkyl residues can respectively be linear or branched and the abovementioned phenyl residues can preferably be substituted with 1, 2, 3, 4 or 5 substituents independently selected from the group consisting of F, Cl, Br, I, —CN, —CF₃, —OH, —NH₂, —O—CF₃, —SH, —O—CH₃, —O—C₂H₅, —O—C₃H₇, methyl, ethyl, n-propyl, isopropyl, n-butyl, 2-butyl, isobutyl and tert-butyl. Particularly preferred substituents can be independently selected from the group consisting of F, Cl, Br, I, —NO₂, —CN, —OH, —SH, —NH₂, —N(CH₃)₂, —N(C₂H₅)₂ and —N(CH₃)(C₂H₅). Suitable C₂₋₁₂-alkenyl residues include, for example, ethenyl, 1-propenyl, 2-propenyl, 1-butenyl, 2-butenyl, 3-butenyl, 1-pentenyl, 2-pentenyl, 3-pentenyl, 4-pentenyl, hexenyl, —CH═C(CH₃)₂, —CH═CH—CH═CH—CH₃ and —CH₂—CH₂—CH═CH₂.

Multiply-substituted alkenyl residues are understood to be alkenyl residues, which are substituted either on different C atoms or on the same C atoms multiple times, preferably twice, e.g. twice on the same C atom as in the case of —CH═CCl₂ or at different points such as in the case of —CCl═CH—(CH₂)—NH₂. Multiple substitution can occur with the same or with different substituents. Suitable substituted alkenyl residues include, for example, —CH═CH—(CH₂)—OH, —CH═CH—(CH₂)—NH₂ and —CH═CH—CN.

In the sense of the present invention the term “alkinyl” denotes acyclic unsaturated hydrocarbon residues, which can be branched or straight-chain as well as unsubstituted or mono- or poly-substituted and have at least one triple bond, preferably 1 or 2 triple bonds with, as in the case of C₂₋₁₂-alkinyl, 2 to 12 (i.e. 2, 3, 4, 5, 6, 7, 8, 9, 10, 11 or 12) C atoms, or as in the case of C₂₋₆-alkinyl, 2 to 6 (i.e. 2, 3, 4, 5 or 6) C atoms. Where one or more of the substituents represents an alkinyl residue or contains an alkinyl residue, which is mono- or multiply-substituted, this can preferably be substituted with optionally 1, 2, 3, 4 or 5, particularly preferably with 1 or 2, substituents independently selected from the group consisting of F, Cl, Br, I, —NO₂, —CN, —OH, —SH, —NH₂, —N(C₁₋₅-alkyl)₂, —N(C₁₋₅-alkyl)(phenyl), —N(C₁₋₅-alkyl)(CH₂-phenyl), —N(C₁₋₅-alkyl)(CH₂—CH₂-phenyl), —NH—C(═O)—O—C₁₋₅-alkyl, —C(═O)—H, —C(═O)—C₁₋₅-alkyl, —C(═O)-phenyl, —C(═S)—C₁₋₅-alkyl, —C(═S)-phenyl, —C(═O)—OH, —C(═O)—O—C₁₋₅-alkyl, —C(═O)—O-phenyl, —C(═O)—NH₂, —C(═O)—NH—C₁₋₅-alkyl, —C(═O)—N(C₁₋₅-alkyl)₂, —S(═O)—C₁₋₅-alkyl, —S(═O)-phenyl, —S(═O)₂—C₁₋₅-alkyl, —S(═O)₂-phenyl, —S(═O)₂—NH₂ and —SO₃H, wherein the abovementioned C₁₋₅-alkyl residues can respectively be linear or branched and the abovementioned phenyl residues can preferably be substituted with 1, 2, 3, 4 or 5 substituents independently selected from the group consisting of F, Cl, Br, I, —CN, —CF₃, —OH, —NH₂, —O—CF₃, —SH, —O—CH₃, —O—C₂H₅, —O—C₃H₇, methyl, ethyl, n-propyl, isopropyl, n-butyl, 2-butyl, isobutyl and tert-butyl. Particularly preferred substituents can be independently selected from the group consisting of F, Cl, Br, I, —NO₂, —CN, —OH, —SH, —NH₂, —N(CH₃)₂, —N(C₂H₅)₂ and —N(CH₃)(C₂H₅).

Suitable C₂₋₁₂-alkinyl residues include, for example, ethinyl, 1-propinyl, 2-propinyl, 1-butinyl, 2-butinyl, 3-butinyl, 1-pentinyl, 2-pentinyl, 3-pentinyl, 4-pentinyl and hexinyl.

Multiply-substituted alkinyl residues are understood to be those alkinyl residues, which are substituted on different C atoms multiple times, e.g. twice on different C atoms as in the case of —CHCl—C≡CCl. Suitable substituted alkinyl residues include, for example, —C≡C—F, —C≡C—Cl and —C≡C—I.

The term “heteroalkyl” refers to an alkyl residue as described above, in which one or more C atoms have been respectively replaced by a heteroatom independently selected from the group consisting of oxygen, sulfur and nitrogen (NH). Heteroalkyl residues can preferably have 1, 2 or 3 heteroatom(s) independently selected from the group consisting of oxygen, sulfur and nitrogen (NH) as chain member(s). Heteroalkyl residues can preferably be 2- to 12-membered, particularly preferred 2- to 6-membered.

Suitable heteroalkyl residues, which may be unsubstituted or mono- or multiply-substituted, include, for example, —CH₂—O—CH₃, —CH₂—O—C₂H₅, —CH₂—O—CH(CH₃)₂, —CH₂—O—C(CH₃)₃, —CH₂—S—CH₃, —CH₂—S—C₂H₅, —CH₂—S—CH(CH₃)₂, —CH₂—S—C(CH₃)₃, —CH₂—NH—CH₃, —CH₂—NH—C₂H₅, —CH₂—NH—CH(CH₃)₂, —CH₂—NH—C(CH₃)₃, —CH₂—CH₂—O—CH₃, —CH₂—CH₂—O—C₂H₅, —CH₂—CH₂—O—CH(CH₃)₂, —CH₂—CH₂—O—C(CH₃)₃, —CH₂—CH₂—S—CH₃, —CH₂—CH₂—S—C₂H₅, —CH₂—CH₂—S—CH(CH₃)₂, —CH₂—CH₂—S—C(CH₃)₃, —CH₂—CH₂—NH—CH₃, —CH₂—CH₂—NH—C₂H₅, —CH₂—CH₂—NH—CH(CH₃)₂, —CH₂—CH₂—NH—C(CH₃)₃, —CH₂—S—CH₂—O—CH₃, —CH₂—O—CH₂—O—C₂H₅, —CH₂—O—CH₂—O—CH(CH₃)₂, —CH₂—S—CH₂—O—C(CH₃)₃, —CH₂—O—CH₂—S—CH₃, —CH₂—O—CH₂—S—C₂H₅, —CH₂—O—CH₂—S—CH(CH₃)₂, —CH₂—NH—CH₂—S—C(CH₃)₃, —CH₂—O—CH₂—NH—CH₃, —CH₂—O—CH₂—NH—C₂H₅, —CH₂—O—CH₂—NH—CH(CH₃)₂, —CH₂—S—CH₂—NH—C(CH₃)₃ and —CH₂—CH₂—C(H)(CH₃)—(CH₂)₃—CH₃.

Suitable substituted heteroalkyl residues include, for example, —(CH₂)—O—(CF₃), —(CH₂)—O—(CHF₂), —(CH₂)—O—(CH₂F), —(CH₂)—S—(CF₃), —(CH₂)—S—(CHF₂), —(CH₂)—S—(CH₂F), —(CH₂)—(CH₂)—O—(CF₃), —(CF₂)—O—(CF₃), —(CH₂)—(CH₂)—S—(CF₃) and —(CH₂)—(CH₂)—(CH₂)—O—(CF₃).

The term “heteroalkenyl” refers to an alkenyl residue as described above, in which one or more C atoms have been respectively replaced by a heteroatom independently selected from the group consisting of oxygen, sulfur and nitrogen (NH). Heteroalkenyl residues preferably may contain 1, 2 or 3 heteroatom(s) independently selected from the group consisting of oxygen, sulfur and nitrogen (NH) as chain member(s). Heteroalkenyl residues can preferably be 2- to 12-membered, particularly preferably 2- to 6-membered.

Suitable heteroalkenyl residues include, for example, —CH₂—O—CH═CH₂, —CH═CH—O—CH═CH—CH₃, —CH₂—CH₂—O—CH═CH₂, —CH₂—S—CH═CH₂, —CH═CH—S—CH═CH—CH₃, —CH₂—CH₂—S—CH═CH₂, —CH₂—NH—CH═CH₂, —CH═CH—NH—CH═CH—CH₃ and —CH₂—CH₂—NH—CH═CH₂.

Suitable substituted heteroalkenyl residues include, for example, —CH₂—O—CH═CH—(CH₂)—OH, —CH₂—S—CH═CH—(CH₂)—NH₂ and —CH₂—NH—CH═CH—CN.

The term “heteroalkinyl” refers to an alkinyl residue as described above, in which one or more C atoms have been respectively replaced by a heteroatom independently selected from the group consisting of oxygen, sulfur and nitrogen (NH). Heteroalkinyl residues preferably may contain 1, 2 or 3 heteroatom(s) independently selected from the group consisting of oxygen, sulfur and nitrogen (NH) as chain member(s). Heteroalkinyl residues can preferably be 2- to 12-membered, particularly preferred 2- to 6-membered.

Suitable heteroalkinyl residues include, for example, —CH₂—O—C≡CH, —CH₂—CH₂—O—C≡CH, —CH₂—O—C≡C—CH₃, —CH₂—CH₂—O—C≡C—CH₃, —CH₂—S—C≡CH, —CH₂—CH₂—S—C≡CH, —CH₂—S—C≡C—CH₃, and —CH₂—CH₂—S—C≡C—CH₃.

Suitable substituted heteroalkinyl residues include, for example, —CH₂—O—C≡C—Cl, —CH₂—CH₂—O—C≡C—I, —CHF—O—C≡C—CH₃, —CHF—CH₂—O—C≡C—CH₃, —CH₂—S—C≡C—Cl, —CH₂—CH₂—S—C≡C—Cl, —CHF—S—C≡C—CH₃, and —CHF—CH₂—S—C≡C—CH₃.

In the sense of the present invention the term “cycloalkyl” refers to a cyclic saturated hydrocarbon residue with preferably 3, 4, 5, 6, 7, 8 or 9 C atoms, particularly preferably 3, 4, 5, 6 or 7 C atoms, and most especially preferred 5 or 6 C atoms, wherein the residue can be unsubstituted or mono-substituted or multiply-substituted with the same or different substituents.

Suitable C₃₋₉-cycloalkyl residues, which can be unsubstituted or mono- or multiply-substituted, include, for example, cyclopropyl, cyclobutyl, cyclopentyl, cyclohexyl, cycloheptyl, cyclooctyl and cyclononyl. Suitable C₃₋₇-cycloalkyl residues are cyclopropyl, cyclobutyl, cyclopentyl, cyclohexyl and cycloheptyl.

In the sense of the present invention the term “cycloalkenyl” means a cyclic unsaturated hydrocarbon residue with preferably 3, 4, 5, 6, 7, 8 or 9 C atoms, particularly preferably 3, 4, 5, 6 or 7 C atoms, and most especially preferably 5 or 6 C atoms, which has at least one double bond, preferably one double bond, and can be unsubstituted or mono-substituted or multiply-substituted with the same or different substituents.

Suitable C₃₋₉-cycloalkenyl residues, which can be unsubstituted or mono- or multiply-substituted, include, for example, cyclobutenyl, cyclopentenyl, cyclohexenyl, cycloheptenyl, cyclononenyl and cyclooctenyl. Suitable C₅₋₆-cycloalkenyl residues include cyclopentenyl and cyclohexenyl.

In the sense of the present invention the term “heterocycloalkyl” means a cyclic saturated hydrocarbon residue with preferably 3, 4, 5, 6, 7, 8 or 9 C atoms, particularly preferably 3, 4, 5, 6 or 7 C atoms, most especially preferably containing 5 or 6 C atoms, in which one or more C atoms have been respectively replaced by a heteroatom independently selected from the group consisting of oxygen, sulfur and nitrogen (NH). Heterocycloalkyl residues preferably contain 1, 2 or 3 heteroatom(s) independently selected from the group consisting of oxygen, sulfur and nitrogen (NH) as ring member(s). A heterocycloalkyl residue can be unsubstituted or mono-substituted or multiply-substituted with the same or different substituents. Heterocycloalkyl residues can preferably be 3- to 9-membered, particularly preferably 3- to 7-membered, most especially preferably 5- to 7-membered.

Suitable 3- to 9-membered heterocycloalkyl residues, which may be unsubstituted or mono- or multiply-substituted, are, for example, imidazolidinyl, tetrahydrofuranyl, tetrahydrothiophenyl, pyrrolidinyl, piperidinyl, morpholinyl, piperazinyl, thiomorpholinyl, tetrahydropyranyl, oxetanyl, azepanyl, azocanyl, diazepanyl, dithiolanyl, (1,3)-dioxolan-2-yl, isoxazolidinyl, isothioazolidinyl, pyrazolidinyl, oxazolidinyl, (1,2,4)-oxadiazolidinyl, (1,2,4)-thiadiazolidinyl, (1,2,4)-triazolidin-3-yl, (1,3,4)-thiadiazolidin-2-yl, (1,3,4)-triazolidin-1-yl, (1,3,4)-triazoldidin-2-yl, tetrahydropyridazinyl, tetrahydropyrimidinyl, tetrahydropyrazinyl, (1,3,5)-tetrahydrotriazinyl, (1,2,4)-tetrahydrotriazin-1-yl, (1,3)-dithian-2-yl and (1,3)-thiazolidinyl. Suitable 5- to 7-membered heterocycloalkyl residues are, for example, imidazolidinyl, tetrahydrofuranyl, tetrahydrothiophenyl, pyrrolidinyl, piperidinyl, morpholinyl, piperazinyl, thiomorpholinyl, tetrahydropyranyl, oxetanyl, azepanyl, diazepanyl and (1,3)-dioxolan-2-yl.

As used herein, the term “heterocycloalkenyl” means a cyclic unsaturated hydrocarbon residue with preferably 4, 5, 6, 7, 8 or 9 C atoms, particularly preferably 4, 5, 6 or 7 C atoms, most especially preferably 5 or 6 C atoms, which has at least one double bond, preferably one double bond and in which one or more C atoms have been respectively replaced by a heteroatom independently selected from the group consisting of oxygen, sulfur and nitrogen (NH). Heterocycloalkenyl residues can preferably have 1, 2 or 3 heteroatom(s) independently selected from the group consisting of oxygen, sulfur and nitrogen (NH) as ring member(s). A heterocycloalkenyl residue can be unsubstituted or mono-substituted or multiply-substituted with the same or different substituents. Heterocycloalkenyl residues can preferably be 4- to 9-membered, particularly preferred 4- to 7-membered, most especially preferred 5- to 7-membered.

Suitable heterocycloalkenyl residues or suitable 5- to 7-membered heterocycloalkenyl residues, which may be unsubstituted or mono- or multiply-substituted, include, for example, (2,3)-dihydrofuranyl, (2,5)-dihydrofuranyl, (2,3)-dihydrothienyl, (2,5)-dihydrothienyl, (2,3)-dihydropyrrolyl, (2,5)-dihydropyrrolyl, (2,3)-dihydroisoxazolyl, (4,5)-dihydroisoxazolyl, (2,5)-dihydroisothiazolyl, (2,3)-dihydropyrazolyl, (4,5)-dihydropyrazolyl, (2,5)-dihydropyrazolyl, (2,3)-dihydrooxazolyl, (4,5)-dihydrooxazolyl, (2,5)-dihydrooxazolyl, (2,3)-dihydrothiazolyl, (4,5)-dihydrothiazolyl, (2,5)-dihydrothiazolyl, (2,3)-dihydroimidazolyl, (4,5)-dihydroimidazolyl, (2,5)-dihydroimidazolyl, (3,4,5,6)-tetrahydropyridin-2-yl, (1,2,5,6)-tetrahydropyridin-1-yl, (1,2)-dihydropyridin-1-yl, (1,4)-dihydropyridin-1-yl, dihydropyranyl and (1,2,3,4)-tetrahydropyridin-1-yl.

In the sense of the present invention a cycloalkyl residue, heterocycloalkyl residue, cycloalkenyl residue or heterocycloalkenyl residue may be condensed (anellated) with an unsubstituted or mono- or poly-substituted mono- or bicyclic ring system. In the sense of the present invention a mono- or bicyclic ring system is understood to mean mono- or bicyclic hydrocarbon residues, which can be saturated, unsaturated or aromatic and can optionally have one or more heteroatoms as ring members. The rings of the abovementioned mono- or bicyclic ring systems are preferably respectively 4-, 5- or 6-membered and can preferably each have possibly 0, 1, 2, 3, 4 or 5 heteroatom(s), particularly preferred possibly 0, 1 or 2 heteroatom(s) as ring members, which are independently selected from the group consisting of oxygen, nitrogen and sulfur. In the case where a bicyclic ring system is present, the different rings, respectively independently of one another, can have a different degree of saturation, i.e. be saturated, unsaturated or aromatic.

If one or more of the substituents contain a monocyclic or bicyclic ring system, which is mono- or multiply-substituted, this can preferably be substituted with optionally 1, 2, 3, 4 or 5, particularly preferred with optionally 1, 2 or 3, substituents, which can be independently selected from the group consisting of F, Cl, Br, I, —CN, —NO₂, —OH, —SH, —NH₂, oxo (═O), thioxo (═S), —C(═O)—OH, C₁₋₅-alkyl, —C₂₋₅-alkenyl, —C₂₋₅-alkinyl, —C≡C—Si(CH₃)₃, —C≡C—Si(C₂H₅)₃, —(CH₂)—O—C₁₋₅-alkyl, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, —S(═O)₂—C₁₋₅-alkyl, —S(═O)—C₁₋₅-alkyl, —NH—C₁₋₅-alkyl, N(C₁₋₅-alkyl)(C₁₋₅-alkyl), —C(═O)—O—C₁₋₅-alkyl, —C(═O)—H, —C(═O)—C₁₋₅-alkyl, —CH₂—O—C(═O)-phenyl, —O—C(═O)-phenyl, —NH—S(═O)₂—C₁₋₅-alkyl, —NH—C(═O)—C₁₋₅-alkyl, —C(═O)—NH₂, —C(═O)—NH—C₁₋₅-alkyl, —C(═O)—N(C₁₋₅-alkyl)₂, pyrazolyl, phenyl, furyl (furanyl), thiadiazolyl, thiophenyl (thienyl) and benzyl, wherein the abovementioned C₁₋₅-alkyl residues can respectively be linear or branched and the cyclic substituents or the cyclic residues of these substituents can themselves be respectively substituted with optionally 1, 2, 3, 4 or 5, preferably with optionally 1, 2, 3 or 4, substituents independently selected from the group consisting of F, Cl, Br, I, —CN, —CF₃, —OH, —NIH2, —O—CF₃, —SH, —O—C₁₋₅-alkyl, —O-phenyl, —O—CH₂-phenyl, —(CH₂)—O—C₁₋₅-alkyl, —S—C₁₋₅-alkyl, —S-phenyl, —S—CH₂-phenyl, —C₁₋₅-alkyl, —C₂₋₅-alkenyl, —C₂₋₅-alkinyl, —C≡C—Si(CH₃)₃, —C≡C—Si(C₂H₅)₃, —C(═O)—O—C₁₋₅-alkyl and —C(═O)—CF₃.

It is particularly preferred that the substituents can be independently selected from the group consisting of F, Cl, Br, I, —CN, —NO₂, —OH, —SH, methyl, ethyl, n-propyl, isopropyl, n-butyl, isobutyl, 2-butyl, tert-butyl, n-pentyl, neo-pentyl, ethenyl, allyl, ethinyl, propinyl, —C≡C—Si(CH₃)₃, —C≡C—Si(C₂H₅)₃, —C≡C—Si(CH₃)₃, —C≡C—Si(C₂H₅)₃, —CH₂—O—CH₃, —CH₂—O—C₂H₅, —OH, —SH, —NH₂, Oxo (═O), —C(═O)—OH, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —O—C(CH₃)₃, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, pyrazolyl, phenyl, —N(CH₃)₂, —N(C₂H₅)₂, —NH—CH₃, —NH—C₂H₅, —CH₂—O—C(═O)-phenyl, —NH—S(═O)₂—CH₃, —C(═O)—O—CH₃, —C(═O)—O—C₂H₅, —C(═O)—O—C(CH₃)₃, —C(═O)—H, —C(═O)—CH₃, —C(═O)—C₂H₅, —NH—C(═O)—CH₃, —NH—C(═O)—C₂H₅, —O—C(═O)-phenyl, —C(═O)—NH₂, —C(═O)—NH—CH₃, —C(═O)—N(CH₃)₂, phenyl, furyl (furanyl), thiadiazolyl, thiophenyl (thienyl) and benzyl, wherein the cyclic substituents or the cyclic residues of these substituents can themselves be substituted with optionally 1, 2, 3, 4 or 5, preferably with possibly 1, 2, 3 or 4, substituents independently selected from the group consisting of F, Cl, Br, I, —CN, —CF₃, —OH, —NH₂, —O—CF₃, —SH, —O—CH₃, —O—C₂H₅, —O—C₃H₇, methyl, ethyl, n-propyl, isopropyl, n-butyl, 2-butyl, isobutyl, tert-butyl, ethenyl, allyl, ethinyl, propinyl, C≡C—Si(CH₃)₃, —C≡C—Si(C₂H₅)₃, —C(═O)—O—C₁₋₅-alkyl and —C(═O)—CF₃.

Suitable cycloalkyl residue, heterocycloalkyl residue, cycloalkenyl residue or heterocycloalkenyl residue, which can be unsubstituted or mono- or multiply-substituted, and are condensed with a mono- or bicyclic ring system, include, for example, (1,2,3,4)-tetrahydroquinolinyl, (1,2,3,4)-tetrahydroisoquinolinyl, (2,3)-dihydro-1H-isoindolyl, indolinyl, (1,2,3,4)-tetrahydronaphthyl, (2,3)-dihydro-benzo[1.4]dioxinyl, benzo[1.3]dioxolyl, (3,4)-dihydro-2H-benzo[1.4]oxazinyl, octahydro-1H-isoindolyl and octahydro-pyrrolo[3,4-c]pyrrolyl.

In the sense of the present invention, a cycloalkyl residue, heterocycloalkyl residue, cycloalkenyl residue or heterocycloalkenyl residue can form a spirocyclic residue with a further cycloalkyl residue, heterocycloalkyl residue, cycloalkenyl residue or heterocycloalkenyl residue via a joint carbon atom. An example of a suitable spirocyclic residue is 8-azaspiro[4.5]decyl residue.

If one or more of the substituents represent a cycloalkyl residue, heterocycloalkyl residue, cycloalkenyl residue or heterocycloalkenyl residue or have such a residue, which is mono- or multiply-substituted, this can preferably be substituted with optionally 1, 2 or 3, substituents independently selected from the group consisting of F, Cl, Br, I, —CN, —CF₃, —OH, —NH₂, —O—CF₃, —SH, —O—C₁₋₅-alkyl, —O-phenyl, —O—CH₂-phenyl, —(CH₂)—O—C₁₋₅-alkyl, —S—C₁₋₅-alkyl, —S-phenyl, —S—CH₂-phenyl, —C₁₋₅-alkyl, —C₂₋₅-alkenyl, —C₂₋₅-alkinyl, —C≡C—Si(CH₃)₃, —C≡C—Si(C₂H₅)₃, —C(═O)—O—C₁₋₅-alkyl, —C(═O)—CF₃, —S(═O)₂—C₁₋₅-alkyl, —S(═O)—C₁₋₅-alkyl, —S(═O)₂-phenyl, oxo (═O), thioxo (═S), —N(C₁₋₅-alkyl)₂, —N(H)(C₁₋₅-alkyl), —NO₂, —S—CF₃, —C(═O)—OH, —NH—S(═O)₂—C₁₋₅-alkyl, —NH—C(═O)—C₁₋₅-alkyl, —NH—C(═O)—CF₃, —C(═O)—H, —C(═O)—C₁₋₅-alkyl, —C(═O)—NH₂, —C(═O)—N(C₁₋₅-alkyl)₂, —C(═O)—N(H)(C₁₋₅-alkyl) and phenyl, wherein the abovementioned C₁₋₅-alkyl residues can respectively be linear or branched and the phenyl residues can be respectively unsubstituted or substituted with 1, 2, 3, 4 or 5, preferably with possibly 1, 2, 3 or 4, substituents independently selected from the group consisting of F, Cl, Br, I, —CN, —CF₃, —OH, —NH₂, —O—CF₃, —SH, —O—C₁₋₅-alkyl, —O-phenyl, —O—CH₂-phenyl, —(CH₂)—O—C₁₋₅-alkyl, —S—C₁₋₅-alkyl, —S-phenyl, —S—CH₂-phenyl, —C₁₋₅-alkyl, —C₂₋₅-alkenyl, —C₂₋₅-alkinyl, —C≡C—Si(CH₃)₃, —C≡C—Si(C₂H₅)₃, —C(═O)—O—C₁₋₅-alkyl and —C(═O)—CF₃.

It is particularly preferred that the substituents can be independently selected from the group consisting of F, Cl, Br, I, —CN, methyl, ethyl, n-propyl, isopropyl, n-butyl, 2-butyl, isobutyl, tert-butyl, ethenyl, allyl, ethinyl, propinyl, —C≡C—Si(CH₃)₃, —C≡C—Si(C₂H₅)₃, —OH, oxo, thioxo, —O—CH₃, —O—C₂H₅, —O—C₃H₇, —(CH₂)—O—CH₃, —(CH₂)—O—C₂H₅, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃, —S—C₂H₅, —S(═O)—CH₃, —S(═O)₂—CH₃, —S(═O)—C₂H₅, —S(═O)₂—C₂H₅, —NH—S(═O)₂—CH₃, —C(═O)—OH, —C(═O)—H; —C(═O)—CH₃, —C(═O)—C₂H₅, —C(═O)—N(CH₃)₂, —C(═O)—NH—CH₃, —C(═O)—NH₂, —NH—C(═O)—CF₃, —NH—C(═O)—CH₃, —NH—C(═O)—C₂H₅, —C(═O)—O—CH₃, —C(═O)—O—C₂H₅, —C(═O)—O—C(CH₃)₃ and phenyl, the phenyl residue can be substituted with 1, 2, 3, 4 or 5, preferably 1, 2 or 3, substituents independently selected from the group consisting of F, Cl, Br, I, —CN, —CF₃, —OH, —NH₂, —O—CF₃, —SH, —O—CH₃, —O—C₂H₅, —O—C₃H₇, methyl, ethyl, n-propyl, isopropyl, n-butyl, 2-butyl, isobutyl, tert-butyl, ethenyl, allyl, ethinyl, propinyl, —C≡C—Si(CH₃)₃, —C≡C—Si(C₂H₅)₃, —C(═O)—O—C₁₋₅-alkyl and —C(═O)—CF₃.

In the sense of the present invention the term “aryl” means a mono- or polycyclic, preferably a mono- or bicyclic, aromatic hydrocarbon residue with preferably 6, 10 or 14 C atoms. An aryl residue can be unsubstituted or mono-substituted or multiply-substituted with the same or different substituents. Suitable aryl residues are, for example, phenyl, 1-naphthyl, 2-naphthyl and anthracenyl. It is particularly preferred that an aryl residue is a phenyl residue.

In the sense of the present invention the term “heteroaryl” means a monocyclic or polycyclic, preferably a mono-, bi- or tricyclic, aromatic hydrocarbon residue with preferably 5, 6, 7, 8, 9, 10, 11, 12, 13 or 14 C atoms, particularly preferably with 5, 6, 9, 10, 13 or 14 C atoms, most especially preferably with 5 or 6 C atoms, in which one or more C atoms have respectively been replaced by a heteroatom independently selected from the group consisting of oxygen, sulfur and nitrogen (NH). Heteroaryl residues can preferably have 1, 2, 3 4 or 5, particularly preferably 1, 2 or 3, heteroatom(s) independently selected from the group consisting of oxygen, sulfur and nitrogen (NH) as ring member(s). A heteroaryl residue can be unsubstituted or mono-substituted or multiply-substituted with the same or different substituents.

Suitable heteroaryl residues include, for example, indolizinyl, benzimidazolyl, tetrazolyl, triazinyl, isoxazolyl, phthalazinyl, carbazolyl, carbolinyl, diaza-naphthyl, thienyl, furyl, pyrrolyl, pyrazolyl, pyrazinyl, pyranyl, triazolyl, pyridinyl, imidazolyl, indolyl, isoindolyl, benzo[b]furanyl, benzo[b]thiophenyl, benzo[d]thiazolyl, benzodiazolyl, benzotriazolyl, benzoxazolyl, benzisoxazolyl, thiazolyl, thiadiazolyl, oxazolyl, oxadiazolyl, isoxazolyl, pyridazinyl, pyrimidinyl, indazolyl, quinoxalinyl, quinazolinyl, quinolinyl, naphthridinyl and isoquinolinyl.

In the sense of the present invention aryl or heteroaryl residues can be condensed (anellated) with a mono- or bicyclic ring system. Examples of aryl residues, which are condensed with a mono- or bicyclic ring system, include (2,3)-dihydrobenzo[b]thiophenyl, (2,3)-dihydro-1H-indenyl, indolinyl, (2,3)-dihydrobenzofuranyl, (2,3)-dihydrobenzo[d]oxazolyl, benzo[d][1,3]dioxolyl, benzo[d][1,3]oxathiolyl, isoindolinyl, (1,3)-dihydroisobenzofuranyl, (1,3)-dihydrobenzo[c]thiophenyl, (1,2,3,4)-tetrahydronaphthyl, (1,2,3,4)-tetrahydroquinolinyl, chromanyl, thiochromanyl, (1,2,3,4)-tetrahydroisoquinolinyl, (1,2,3,4)-tetrahydroquinoxalinyl, (3,4)-dihydro-2H-benzo[b][1,4]oxazinyl, (3,4)-dihydro-2H-benzo[b][1,4]thiazinyl, (2,3)-dihydrobenzo[b][1,4]dioxinyl, (2,3)-dihydrobenzo[b][1,4]oxathiinyl, (6,7,8,9)-tetrahydro-5H-benzo[7]annulenyl, (2,3,4,5)-tetrahydro-1H-benzo[b]azepinyl and (2,3,4,5)-tetrahydro-1H-benzo[c]azepinyl.

Unless specified otherwise, where one or more of the substituents represent an aryl or heteroaryl residue or contain an aryl or heteroaryl residue, which is mono- or multiply-substituted, these aryl or heteroaryl residues can preferably be substituted with optionally 1, 2, 3, 4 or 5, particularly preferably with optionally 1, 2 or 3, substituents independently selected from the group consisting of F, Cl, Br, I, —CN, —NO₂, —OH, —SH, —NH₂, —C(═O)—OH, —C₁₋₅-alkyl, —(CH₂)—O—C₁₋₅-alkyl, —C₂₋₅-alkenyl, —C₂₋₅-alkinyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, —S(═O)₂—C₁₋₅-alkyl, —S(═O)—C₁₋₅-alkyl, —NH—C₁₋₅-alkyl, N(C₁₋₅alkyl)₂, —C(═O)—O—C₁₋₅-alkyl, —C(═O)—O-phenyl, —C(═O)—H; —C(═O)—C₁₋₅-alkyl, —CH₂—O—C(═O)-phenyl, —O—C(═O)—C₁₋₅-alkyl, —O—C(═O)-phenyl, —NH—S(═O)₂—C₁₋₅-alkyl, —NH—C(═O)—C₁₋₅-alkyl, —C(═O)—NH₂, —C(═O)—NH—C₁₋₅-alkyl, —C(═O)—N(C₁₋₅-alkyl)₂, —C(═O)—N(C₁₋₅-alkyl)(phenyl), —C(═O)—NH-phenyl, cyclopropyl, cyclobutyl, cyclopentyl, cyclohexyl, piperazinyl, pyrrolidinyl, piperidinyl, pyrazolyl, phenyl, furyl (furanyl), thiazolyl, thiadiazolyl, thiophenyl (thienyl), benzyl and phenethyl, wherein the abovementioned C₁₋₅-alkyl residues can respectively be linear or branched and the cyclic substituents or the cyclic residues of these substituents can themselves be substituted with optionally 1, 2, 3, 4 or 5, preferably with optionally 1, 2, 3 or 4, substituents independently selected from the group consisting of F, Cl, Br, I, —CN, —NO₂, —OH, —SH, —NH₂, —C(═O)—OH, —C₁₋₅-alkyl, —(CH₂)—O—C₁₋₅-alkyl, —C₂₋₅-alkenyl, —C₂₋₅-alkinyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂ and —S—CH₂F.

It is particularly preferred if the substituents are each independently selected from the group consisting of F, Cl, Br, I, —CN, —NO₂, —OH, —SH, methyl, ethyl, n-propyl, isopropyl, n-butyl, isobutyl, 2-butyl, tert-butyl, n-pentyl, neo-pentyl, ethenyl, allyl, ethinyl, propinyl, —C≡C—Si(CH₃)₃, —C≡C—Si(C₂H₅)₃, —CH₂—O—CH₃, —CH₂—O—C₂H₅, —OH, —SH, —NH₂, —C(═O)—OH, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —O—C(CH₃)₃, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, pyrazolyl, phenyl, —N(CH₃)₂, —N(C₂H₅)₂, —NH—CH₃, —NH—C₂H₅, —CH₂—O—C(═O)-phenyl, —NH—S(═O)₂—CH₃, —C(═O)—O—CH₃, —C(═O)—O—C₂H₅, —C(═O)—O—C(CH₃)₃, —C(═O)—H, —C(═O)—CH₃, —C(═O)—C₂H₅, —NH—C(═O)—CH₃, —NH—C(═O)—C₂H₅, —O—C(═O)-phenyl, —C(═O)—NH₂, —C(═O)—NH—CH₃, —C(═O)—N(CH₃)₂, —C(═O)—O—CH(CH₃)₂, —C(═O)—O—(CH₂)₃—CH₃, —C(═O)—O-phenyl, —O—C(═O)—CH₃, —O—C(═O)—C₂H₅, —C(═O)—NH—C₂H₅, —C(═O)—NH—C(CH₃)₃, —C(═O)—N(C₂H₅)₂, —C(═O)—NH-phenyl, —C(═O)—N(CH₃)-phenyl, —C(═O)—N(C₂H₅)-phenyl, cyclopropyl, cyclobutyl, cyclopentyl, cyclohexyl, piperazinyl, pyrrolidinyl, piperidinyl, phenyl, furyl (furanyl), thiadiazolyl, thiophenyl (thienyl) and benzyl, wherein the cyclic substituents or the cyclic residues of these substituents can themselves be respectively substituted with optionally 1, 2, 3, 4 or 5, preferably with possibly 1, 2, 3 or 4, substituents independently selected from the group consisting of F, Cl, Br, I, —CN, —NO₂, —OH, —SH, —NH₂, —C(═O)—OH, methyl, ethyl, n-propyl, isopropyl, n-butyl, isobutyl, 2-butyl, tert-butyl, n-pentyl, neo-pentyl, ethenyl, allyl, ethinyl, propinyl, —C≡C—Si(CH₃)₃, —C≡C—Si(C₂H₅)₃, —CH₂—O—CH₃, —CH₂—O—C₂H₅, —S—CH₃, —S—C₂H₅, —S(═O)—CH₃, —S(═O)₂—CH₃, —S(═O)—C₂H₅, —S(═O)₂—C₂H₅, —O—CH₃, —O—C₂H₅, —O—C₃H₇, —O—C(CH₃)₃, —CF₃, —CHF₂, —CH₂F, —O—CF₃, —O—CHF₂, —O—CH₂F, —C(═O)—CF₃, —S—CF₃, —S—CHF₂ and —S—CH₂F.

Most particularly preferred a substituted aryl residue can be selected from the group consisting of 2-methyl-phenyl, 3-methyl-phenyl, 4-methyl-phenyl, 2-fluoro-phenyl, 3-fluoro-phenyl, 4-fluoro-phenyl, 2-cyano-phenyl, 3-cyano-phenyl, 4-cyano-phenyl, 2-hydroxy-phenyl, 3-hydroxy-phenyl, 4-hydroxy-phenyl, 2-amino-phenyl, 3-amino-phenyl, 4-amino-phenyl, 2-dimethylamino-phenyl, 3-dimethylamino-phenyl, 4-dimethylamino-phenyl, 2-methylamino-phenyl, 3-methylamino-phenyl, 4-methylamino-phenyl, 2-acetyl-phenyl, 3-acetyl-phenyl, 4-acetyl-phenyl, 2-methylsulfinyl-phenyl, 3-methylsulfinyl-phenyl, 4-methylsulfinyl-phenyl, 2-methylsulfonyl-phenyl, 3-methylsulfonyl-phenyl, 4-methylsulfonyl-phenyl, 2-methoxy-phenyl, 3-methoxy-phenyl, 4-methoxy-phenyl, 2-chloro-phenyl, 3-chloro-phenyl, 4-chloro-phenyl, 2-ethoxy-phenyl, 3-ethoxy-phenyl, 4-ethoxyphenyl, 2-trifluoromethyl-phenyl, 3-trifluoromethyl-phenyl, 4-trifluoromethyl-phenyl, 2-difluoromethyl-phenyl, 3-difluoromethyl-phenyl, 4-difluoromethyl-phenyl, 2-fluoromethyl-phenyl, 3-fluoromethyl-phenyl, 4-fluoromethyl-phenyl, 2-nitro-phenyl, 3-nitro-phenyl, 4-nitro-phenyl, 2-ethyl-phenyl, 3-ethyl-phenyl, 4-ethyl-phenyl, 2-propyl-phenyl, 3-propyl-phenyl, 4-propyl-phenyl, 2-isopropyl-phenyl, 3-isopropyl-phenyl, 4-isopropyl-phenyl, 2-tert-butyl-phenyl, 3-tert-butyl-phenyl, 4-tert-butyl-phenyl, 2-carboxyphenyl, 3-carboxy-phenyl, 4-carboxyphenyl, 2-ethenyl-phenyl, 3-ethenyl-phenyl, 4-ethenyl-phenyl, 2-ethinyl-phenyl, 3-ethinyl-phenyl, 4-ethinyl-phenyl, 2-allyl-phenyl, 3-allyl-phenyl, 4-allyl-phenyl, 2-trimethylsilanylethinyl-phenyl, 3-trimethylsilanylethinyl-phenyl, 4-trimethylsilanylethinyl-phenyl, 2-formyl-phenyl, 3-formyl-phenyl, 4-formyl-phenyl, 2-acetamino-phenyl, 3-acetamino-phenyl, 4-acetamino-phenyl, 2-dimethylaminocarbonyl-phenyl, 3-dimethylaminocarbonyl-phenyl, 4-dimethylaminocarbonyl-phenyl, 2-methoxymethyl-phenyl, 3-methoxymethyl-phenyl, 4-methoxymethyl-phenyl, 2-ethoxymethyl-phenyl, 3-ethoxymethyl-phenyl, 4-ethoxymethyl-phenyl, 2-aminocarbonyl-phenyl, 3-aminocarbonyl-phenyl, 4-aminocarbonyl-phenyl, 2-methylaminocarbonyl-phenyl, 3-methylaminocarbonyl-phenyl, 4-methylaminocarbonyl-phenyl, 2-carboxymethylester-phenyl, 3-carboxymethylester-phenyl, 4-carboxymethylester-phenyl, 2-carboxyethylester-phenyl, 3-carboxyethylester-phenyl, 4-carboxyethylester-phenyl, 2-carboxy-tert-butylester-phenyl, 3-carboxy-tert-butylester-phenyl, 4-carboxy-tert-butylester-phenyl, 2-methylmercapto-phenyl, 3-methylmercapto-phenyl, 4-methylmercapto-phenyl, 2-ethylmercapto-phenyl, 3-ethylmercapto-phenyl, 4-ethylmercaptophenyl, 2-biphenyl, 3-biphenyl, 4-biphenyl, 2-bromophenyl, 3-bromophenyl, 4-bromophenyl, 2-iodo-phenyl, 3-iodophenyl, 4-iodophenyl, 2-trifluoromethoxy-phenyl, 3-trifluoromethoxy-phenyl, 4-trifluoromethoxy-phenyl, 2-fluoro-3-trifluoromethylphenyl, 2-fluoro-4-methyl-phenyl, (2,3)-difluorophenyl, (2,3)-dimethyl-phenyl, (2,3)-dichlorophenyl, 3-fluoro-2-trifluoromethylphenyl, (2,4)-dichloro-phenyl, (2,4)-difluorophenyl, 4-fluoro-2-trifluoromethyl-phenyl, (2,4)-dimethoxyphenyl, 2-chloro-4-fluoro-phenyl, 2-chloro-4-nitro-phenyl, 2-chloro-4-methyl-phenyl, 2-chloro-5-trifluoromethyl-phenyl, 2-chloro-5-methoxy-phenyl, 2-bromo-5-trifluoromethyl-phenyl, 2-bromo-5-methoxy-phenyl, (2,4)-dibromo-phenyl, (2,4)-dimethyl-phenyl, 2-fluoro-4-trifluoromethyl-phenyl, (2,5)-difluoro-phenyl, 2-fluoro-5-trifluoromethyl-phenyl, 5-fluoro-2-trifluoromethyl-phenyl, 5-chloro-2-trifluoromethyl-phenyl, 5-bromo-2-trifluoromethyl-phenyl, (2,5)-dimethoxy-phenyl, (2,5)-bis-trifluoromethyl-phenyl, (2,5)-dichloro-phenyl, (2,5)-dibromo-phenyl, 2-methoxy-5-nitro-phenyl, 2-fluoro-6-trifluoromethyl-phenyl, (2,6)-dimethoxy-phenyl, (2,6)-dimethyl-phenyl, (2,6)-dichloro-phenyl, 2-chloro-6-fluoro-phenyl, 2-bromo-6-chloro-phenyl, 2-bromo-6-fluoro-phenyl, (2,6)-difluoro-phenyl, (2,6)-difluoro-3-methyl-phenyl, (2,6)-dibromo-phenyl, (2,6)-dichlorophenyl, 3-chloro-2-fluoro-phenyl, 3-chloro-5-methyl-phenyl, (3,4)-dichlorophenyl, (3,4)-dimethyl-phenyl, 3-methyl-4-methoxy-phenyl, 4-chloro-3-nitro-phenyl, (3,4)-dimethoxy-phenyl, 4-fluoro-3-trifluoromethylphenyl, 3-fluoro-4-trifluoromethyl-phenyl, (3,4)-difluoro-phenyl, 3-cyano-4-fluoro-phenyl, 3-cyano-4-methyl-phenyl, 3-cyano-4-methoxy-phenyl, 3-bromo-4-fluoro-phenyl, 3-bromo-4-methyl-phenyl, 3-bromo-4-methoxy-phenyl, 4-chloro-2-fluoro-phenyl, 4-chloro-3-trifluoromethyl, 4-bromo-3-methyl-phenyl, 4-bromo-5-methyl-phenyl, 3-chloro-4-fluoro-phenyl, 4-fluoro-3-nitro-phenyl, 4-bromo-3-nitro-phenyl, (3,4)-dibromo-phenyl, 4-chloro-3-methyl-phenyl, 4-bromo-3-methyl-phenyl, 4-fluoro-3-methyl-phenyl, 3-fluoro-4-methyl-phenyl, 3-fluoro-5-methyl-phenyl, 2-fluoro-3-methyl-phenyl, 4-methyl-3-nitro-phenyl, (3,5)-dimethoxy-phenyl, (3,5)-dimethyl-phenyl, (3,5)-bis-trifluoromethyl-phenyl, (3,5)-difluoro-phenyl, (3,5)-dinitro-phenyl, (3,5)-dichloro-phenyl, 3-fluoro-5-trifluoromethyl-phenyl, 5-fluoro-3-trifluoromethyl-phenyl, (3,5)-dibromo-phenyl, 5-chloro-4-fluoro-phenyl, 5-chloro-4-fluoro-phenyl, 5-bromo-4-methyl-phenyl, (2,3,4)-trifluorophenyl, (2,3,4)-trichlorophenyl, (2,3,6)-trifluoro-phenyl, 5-chloro-2-methoxy-phenyl, (2,3)-difluoro-4-methyl, (2,4,5)-trifluoro-phenyl, (2,4,5)-trichloro-phenyl, (2,4)-dichloro-5-fluoro-phenyl, (2,4,6)-trichloro-phenyl, (2,4,6)-trimethylphenyl, (2,4,6)-trifluoro-phenyl, (2,4,6)-trimethoxy-phenyl, (3,4,5)-trimethoxy-phenyl, (2,3,4,5)-tetrafluoro-phenyl, 4-methoxy-(2,3,6)-trimethyl-phenyl, 4-methoxy-(2,3,6)-trimethyl-phenyl, 4-chloro-2,5-dimethyl-phenyl, 2-chloro-6-fluoro-3-methyl-phenyl, 6-chloro-2-fluoro-3-methyl, (2,4,6)-trimethylphenyl and (2,3,4,5,6)-pentafluoro-phenyl.

Most particularly preferred, a substituted heteroaryl residue can be selected from the group consisting of 3-methyl-pyrid-2-yl, 4-methyl-pyrid-2-yl, 5-methyl-pyrid-2-yl, 6-methyl-pyrid-2-yl, 2-methyl-pyrid-3-yl, 4-methyl-pyrid-3-yl, 5-methyl-pyrid-3-yl, 6-methyl-pyrid-3-yl, 2-methyl-pyrid-4-yl, 3-methyl-pyrid-4-yl, 3-fluoro-pyrid-2-yl, 4-fluoro-pyrid-2-yl, 5-fluoro-pyrid-2-yl, 6-fluoro-pyrid-2-yl, 3-chloro-pyrid-2-yl, 4-chloro-pyrid-2-yl, 5-chloro-pyrid-2-yl, 6-chloro-pyrid-2-yl, 3-trifluoromethyl-pyrid-2-yl, 4-trifluoromethyl-pyrid-2-yl, 5-trifluoromethyl-pyrid-2-yl, 6-trifluoromethyl-pyrid-2-yl, 3-methoxy-pyrid-2-yl, 4-methoxy-pyrid-2-yl, 5-methoxy-pyrid-2-yl, 6-methoxy-pyrid-2-yl, 4-methyl-thiazol-2-yl, 5-methyl-thiazol-2-yl, 4-trifluoromethyl-thiazol-2-yl, 5-trifluoromethyl-thiazol-2-yl, 4-chloro-thiazol-2-yl, 5-chloro-thiazol-2-yl, 4-bromo-thiazol-2-yl, 5-bromo-thiazol-2-yl, 4-fluoro-thiazol-2-yl, 5-fluoro-thiazol-2-yl, 4-cyano-thiazol-2-yl, 5-cyano-thiazol-2-yl, 4-methoxy-thiazol-2-yl, 5-methoxy-thiazol-2-yl, 4-methyl-oxazol-2-yl, 5-methyl-oxazol-2-yl, 4-trifluoromethyl-oxazol-2-yl, 5-trifluoromethyl-oxazol-2-yl, 4-chloro-oxazol-2-yl, 5-chloro-oxazol-2-yl, 4-bromo-oxazol-2-yl, 5-bromo-oxazol-2-yl, 4-fluoro-oxazol-2-yl, 5-fluoro-oxazol-2-yl, 4-cyano-oxazol-2-yl, 5-cyano-oxazol-2-yl, 4-methoxy-oxazol-2-yl, 5-methoxy-oxazol-2-yl, 2-methyl-(1,2,4)-thiadiazol-5-yl, 2-trifluoromethyl-(1,2,4)-thiadiazol-5-yl, 2-chloro-(1,2,4)-thiadiazol-5-yl, 2-fluoro-(1,2,4)-thiadiazol-5-yl, 2-methoxy-(1,2,4)-thiadiazol-5-yl, 2-cyano-(1,2,4)-thiadiazol-5-yl, 2-methyl-(1,2,4)-oxadiazol-5-yl, 2-trifluoromethyl-(1,2,4)-oxadiazol-5-yl, 2-chloro-(1,2,4)-oxadiazol-5-yl, 2-fluoro-(1,2,4)-oxadiazol-5-yl, 2-methoxy-(1,2,4)-oxadiazol-5-yl and 2-cyano-(1,2,4)-oxadiazol-5-yl.

In the sense of the present invention the term “alkylene” denotes acyclic saturated hydrocarbon chains, which link an aryl, heteroaryl, cycloalkyl, heterocycloalkyl, cycloalkenyl or heterocycloalkenyl residue to the compounds of formula I or to another substituent. Alkylene chains can be branched or straight-chain and also unsubstituted or mono- or poly-substituted with, as in the case of C₁₋₁₂ alkylene, 1 to 12 (i.e. 1, 2, 3, 4, 5, 6, 7, 8, 9, 10, 11 or 12) C atoms, with, as in the case of C₁₋₆ alkylene, 1 to 6 (i.e. 1, 2, 3, 4, 5 or 6) C atoms, or with, as in the case of C₁₋₃ alkylene, 1 to 3 (i.e. 1, 2 or 3) C atoms. Examples of suitable C₁₋₆ alkylene groups are —(CH₂)—, —(CH₂)₂—, —C(H)(CH₃)—, —(CH₂)₃—, —(CH₂)₄—, —(CH₂)₅—, —C(CH₃)₂—, —C(H)(CH₃)—, —C(H)(C(H)(CH₃)₂)— and C(C₂H₅)(H)—. Examples of C₁₋₃-alkylene groups include —(CH₂)—, —(CH₂)₂— and —(CH₂)₃—.

In the sense of the present invention the term “alkenylene” denotes acyclic unsaturated hydrocarbon chains, which link an aryl, heteroaryl, cycloalkyl, heterocycloalkyl, cycloalkenyl or heterocycloalkenyl residue to the compounds of the general formula I or to another substituent. Alkenylene chains have at least one double bond, preferably 1, 2 or 3 double bonds, and can be branched or straight-chain and also unsubstituted or mono- or poly-substituted with, as in the case of C₂₋₁₂ alkenylene, 2 to 12 (i.e. 2, 3, 4, 5, 6, 7, 8, 9, 10, 11 or 12) C atoms with, as in the case of C₂₋₆ alkenylene, 2 to 6 (i.e. 2, 3, 4, 5 or 6) C atoms, or with, as in the case of C₂₋₃ alkenylene, 2 to 3 (i.e. 2 or 3) C atoms. C₂₋₃ alkenylene groups such as —CH═CH— and ═CH₂—CH═CH— are specified by way of example.

In the sense of the present invention the term “alkinylene” refers to acyclic unsaturated hydrocarbon chains, which link an aryl, heteroaryl, cycloalkyl, heterocycloalkyl, cycloalkenyl or heterocycloalkenyl residue to the compounds of the general formula I or to another substituent. Alkinylene chains have at least one triple bond, preferably 1 or 2 triple bonds, and can be branched or straight-chain and also unsubstituted or mono- or poly-substituted with, as in the case of C₂₋₁₂ alkinylene, 2 to 12 (i.e. 2, 3, 4, 5, 6, 7, 8, 9, 10, 11 or 12) C atoms, with, as in the case of C₂₋₆ alkinylene, 2 to 6 (i.e. 2, 3, 4, 5 or 6) C atoms, or with, as in the case of C₂₋₃ alkinylene, 2 to 3 (i.e. 2 or 3) C atoms. C₂₋₃ alkinylene groups such as —CH≡C— and —CH₂—C≡C— are specified by way of example.

The term “heteroalkylene” refers to an alkylene chain as described above, in which one or more C atoms have been respectively replaced by a heteroatom independently selected from the group consisting of oxygen, sulfur and nitrogen (NH). Heteroalkylene groups can preferably have 1, 2 or 3 heteroatom(s), particularly preferred one heteroatom, selected from the group consisting of oxygen, sulfur and nitrogen (NH) as chain member(s). Heteroalkylene groups can preferably be 2- to 12-membered, particularly preferred 2- to 6-membered, most particularly preferred 2- or 3-membered.

Heteroalkylene groups such as —(CH₂)—O—, —(CH₂)₂—O—, —(CH₂)₃—O—, —(CH₂)₄—O—, —O—(CH₂)—, —O—(CH₂)₂—, —O—(CH₂)₃—, —O—(CH₂)₄—, —C(C₂H₅)(H)—O—, —O—C(C₂H₅)(H)—, —CH₂—O—CH₂—, —CH₂—S—CH₂—, —CH₂—NH—CH₂—, —CH₂—NH— and —CH₂—CH₂—NH—CH₂—CH₂ are specified by way of example.

The term “heteroalkenylene” refers to an alkenylene chain as described above, in which one or more C atoms have been respectively replaced by a heteroatom independently selected from the group consisting of oxygen, sulfur and nitrogen (NH). Heteroalkenylene groups can preferably have 1, 2 or 3 heteroatom(s), particularly preferred 1 heteroatom, selected from the group consisting of oxygen, sulfur and nitrogen (NH) as chain member(s). Heteroalkenylene groups can preferably be 2- to 12-membered, particularly preferred 2- to 6-membered, most particularly preferred 2- or 3-membered. Heteroalkenylene groups such as —CH═CH—NH—, CH═CH—O— and —CH═CH—S— are specified by way of example.

If one or more of the substituents represents an alkylene, alkenylene, alkinylene, heteroalkylene or heteroalkenylene group or contains such a group, which is mono- or multiply-substituted, this can preferably be substituted with optionally 1, 2, 3, 4 or 5, particularly preferably with optionally 1, 2 or 3 substituents independently selected from the group consisting of phenyl, F, Cl, Br, I, —NO₂, —CN, —OH, —O-phenyl, —O—CH₂-phenyl, —SH, —S-phenyl, —S—CH₂-phenyl, —NH₂, —N(C₁₋₅-alkyl)₂, —NH-phenyl, —N(C₁₋₅-alkyl)(phenyl), —N(C₁₋₅-alkyl)(CH₂-phenyl), —N(C₁₋₅-alkyl)(CH₂—CH₂-phenyl), —C(═O)—H, —C(═O)—C₁₋₅-alkyl, —C(═O)-phenyl, —C(═S)—C₁₋₅-alkyl, —C(═S)-phenyl, —C(═O)—OH, —C(═O)—O—C₁₋₅-alkyl, —C(═O)—O-phenyl, —C(═O)—NH₂, —C(═O)—NH—C₁₋₅-alkyl, —C(═O)—N(C₁₋₅-alkyl)₂, —S(═O)—C₁₋₅-alkyl, —S(═O)-phenyl, —S(═O)₂—C₁₋₅-alkyl, —S(═O)₂-phenyl, —S(═O)₂—NH₂ and —SO₃H, wherein the abovementioned C₁₋₅-alkyl residues can respectively be linear or branched and the abovementioned phenyl residues can be substituted with 1, 2, 3, 4 or 5, preferably with 1, 2, 3 or 4, substituents independently selected from the group consisting of F, Cl, Br, I, —CN, —NO₂, —OH, —SH, —NH₂, —C(═O)—OH, —C₁₋₅-alkyl, —(CH₂)—O—C₁₋₅-alkyl, —C₂₋₅-alkenyl, —C₂₋₅-alkinyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂ and —S—CH₂F.

It is particularly preferred that alkylene, alkenylene, alkinylene, heteroalkylene or heteroalkenylene groups with 1, 2 or 3 substituents can be independently selected from the group consisting of phenyl, F, Cl, Br, I, —NO₂, —CN, —OH, —O-phenyl, —SH, —S-phenyl, —NH₂, —N(CH₃)₂, —N(C₂H₅)₂ and —N(CH₃)(C₂H₅), wherein the phenyl residue can be substituted with 1, 2, 3, 4 or 5 substituents independently selected from the group consisting of F, Cl, Br, I, —OH, —SH, —NO₂, —CN, —O—CH₃, —O—CF₃ and —O—C₂H₅.

Substituted 4-amino-quinazoline compounds of the foregoing formula I are preferred in which T, U, V and optionally W together with two carbon atoms form a ring selected from the group consisting of

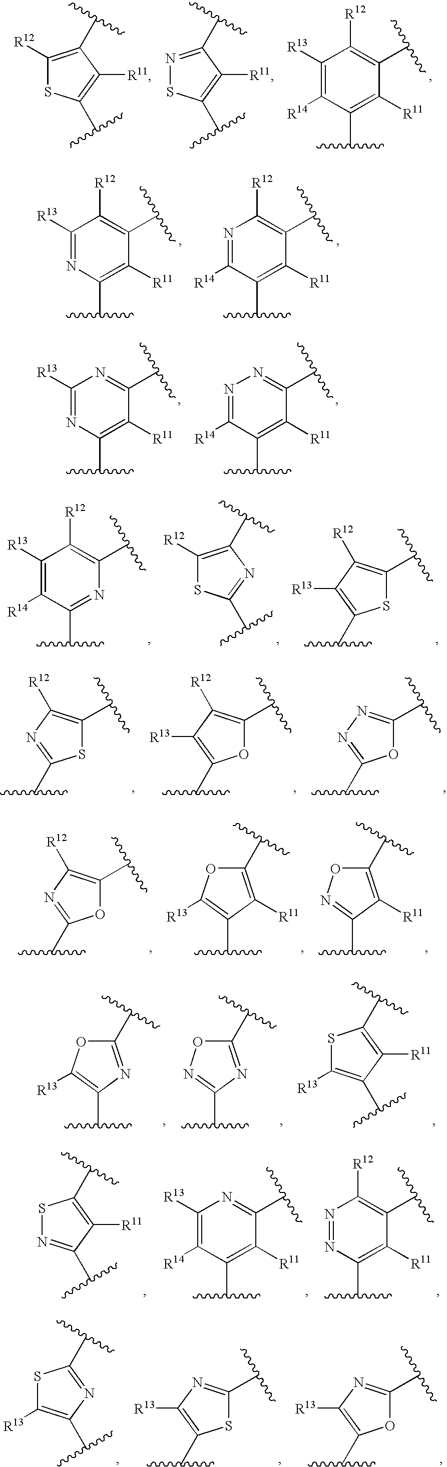

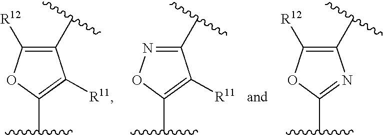

and the meaning of the other residues in each case is as specified above, in each case optionally in the form of one of their pure stereoisomers, in particular enantiomers or diastereomers, their racemates, or in the form of a mixture of stereoisomers, in particular the enantiomers and/or diastereomers, in any mixture ratio, or respectively in the form of corresponding salts or respectively in the form of corresponding solvates.

Substituted 4-amino quinazoline compounds corresponding to the foregoing formula I are also preferred in which:
R¹ and R² each independently represent H; —C(═O)—OH; —C(═O)—H; —C(═O)—R¹⁵; —C(═O)—O—R¹⁶; —C(═O)—NH₂; —C(═O)—NH—R¹⁷; —C(═S)—NH—R¹⁷; —C(═O)—NR¹⁸R¹⁹; —C(═S)—NR¹⁸R¹⁹; —S(═O)—R²⁰; —S(═O)₂—R²¹; C₁₋₆-alkyl, C₂₋₆-alkenyl or C₂₋₆-alkinyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; 2- to 6-membered heteroalkyl, 2- to 6-membered heteroalkenyl or 2- to 6-membered heteroalkinyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃, and has 1 or 2 heteroatom(s) independently selected from the group consisting of oxygen, sulfur and nitrogen (NH) as chain member(s); C₃₋₇-cycloalkyl, C₅₋₆-cycloalkenyl, 5- to 7-membered heterocycloalkyl or 5- to 7-membered heterocycloalkenyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅, and can possibly have 1 or 2 heteroatom(s) independently selected from the group consisting of oxygen, sulfur and nitrogen (NH) as ring member(s); or represent a residue selected from the group consisting of phenyl, naphthyl and anthracenyl, which can be respectively bonded via a C₁₋₃-alkylene, C₂₋₃-alkenylene or C₂₋₃-alkinylene group and/or is unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, piperazinyl, pyrrolidinyl, piperidinyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅; or
R¹ and R² together with the nitrogen atom to which they are bound form a 5- to 7-membered heterocycloalkyl or 5- to 7-membered heterocycloalkenyl group, which may be unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅, can have 1 or 2 further heteroatom(s) independently selected from the group consisting of oxygen, sulfur and nitrogen (NH) as ring member(s);
   and the meaning of the remaining residues in each case is as specified above, in each case optionally in the form of one of their pure stereoisomers, in particular enantiomers or diastereomers, their racemates, or in the form of a mixture of stereoisomers, in particular the enantiomers and/or diastereomers, in any mixture ratio, or respectively in the form of corresponding salts or respectively in the form of corresponding solvates.

Substituted 4-amino quinazoline compounds corresponding to the foregoing formula I are also preferred in which:
R³ represents H; F; Cl; Br; I; —NO₂; —CN; —NH₂; —OH; —SH; —C(═O)—OH; —C(═O)—H; —C(═O)—R¹⁵; —C(═O)—O—R¹⁶; —C(═O)—NH₂; —C(═O)—NH—R¹⁷; —C(═S)—NH—R¹⁷; —C(═O)—NR¹⁸R¹⁹; —C(═S)—NR¹⁸R¹⁹; —NH—R²²; —NR²³R²⁴; —O—R²⁵; —S—R²⁶; C₁₋₆-alkyl, C₂₋₆-alkenyl or C₂₋₆-alkinyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃ and —C(═O)—O—C₂H₅; C₃₋₇-cycloalkyl, C₅₋₆-cycloalkenyl, 5- to 7-membered heterocycloalkyl or 5- to 7-membered heterocycloalkenyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅, and can optionally contain 1 or 2 heteroatom(s) independently selected from the group consisting of oxygen, sulfur and nitrogen (NH) as ring member(s); or represents a residue selected from the group consisting of phenyl, pyrrolyl, furanyl, thiophenyl, pyrazolyl, imidazolyl, thiazolyl, thiadiazolyl, triazolyl, oxazolyl, oxadiazolyl, isoxazolyl, pyridinyl, pyridazinyl, pyrimidinyl, pyrazinyl and pyranyl, which can respectively be bonded by a C₁₋₃-alkylene, C₂₋₃-alkenylene or C₂₋₃-alkinylene group and/or be unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅; and
   the meaning of each of the remaining residues is as specified above, in each case optionally in the form of one of their pure stereoisomers, in particular enantiomers or diastereomers, their racemates, or in the form of a mixture of stereoisomers, in particular the enantiomers and/or diastereomers, in any mixture ratio, or respectively in the form of corresponding salts or respectively in the form of corresponding solvates.

Substituted 4-amino quinazoline compounds of the foregoing formula I are further preferred in which:
R⁴, R⁵ and R⁶ each independently represent H; F; Cl; Br; I; —NO₂; —CN; —NH₂; —OH; —SH; —C(═O)—OH; —C(═O)—H; —C(═O)—NH₂; —NH—R²²; —NR²³R²⁴; —O—R²⁵; —S—R²⁶; or C₁₋₆-alkyl, C₂₋₆-alkenyl or C₂₋₆-alkinyl, which is respectively unsubstituted or substituted with optionally 1, 2, 3, 4 or 5 substituents independently selected from the group consisting of F, Cl, Br, I, —NO₂, —CN, —OH, —SH and —NH₂;
   and the meaning of the remaining residues in each case is as specified above, in each case optionally in the form of one of their pure stereoisomers, in particular enantiomers or diastereomers, their racemates, or in the form of a mixture of stereoisomers, in particular the enantiomers and/or diastereomers, in any mixture ratio, or respectively in the form of corresponding salts or respectively in the form of corresponding solvates.

Substituted 4-amino quinazoline compounds corresponding to the foregoing formula I are additionally preferred in which:
R⁷ and R⁸ each independently represent H; F; Cl; Br; I; —NO₂; —CN; —NH₂; —OH; —SH; —C(═O)—OH; —C(═O)—H; —C(═O)—NH₂; —NH—R²²; —NR²³R²⁴; —O—R²⁵; —S—R²⁶; or C₁₋₆-alkyl, C₂₋₆-alkenyl or C₂₋₆-alkinyl, which is respectively unsubstituted or substituted with optionally 1, 2, 3, 4 or 5 substituents independently selected from the group consisting of F, Cl, Br, I, —NO₂, —CN, —OH, —SH and —NH₂;
   and the meaning of the remaining residues in each case is as specified above, in each case optionally in the form of one of their pure stereoisomers, in particular enantiomers or diastereomers, their racemates, or in the form of a mixture of stereoisomers, in particular the enantiomers and/or diastereomers, in any mixture ratio, or respectively in the form of corresponding salts or respectively in the form of corresponding solvates.

Substituted 4-amino quinazoline compounds corresponding to the foregoing formula I are likewise preferred, wherein
R⁹ represents H; —C(═O)—OH; —C(═O)—H; —C(═O)—R¹⁵; —C(═O)—O—R¹⁶; —C(═O)—NH₂; —C(═O)—NH—R¹⁷; —C(═S)—NH—R¹⁷; —C(═O)—NR¹⁸R¹⁹; —C(═S)—NR¹⁸R¹⁹; —S(═O)—R²⁰; —S(═O)₂—R²¹; C₁₋₆-alkyl, C₂₋₆-alkenyl or C₂₋₆-alkinyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; 2- to 6-membered heteroalkyl, 2- to 6-membered heteroalkenyl or 2- to 6-membered heteroalkinyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂ and —N(CH₃)(C₂H₅) and contains 1 or 2 heteroatom(s) independently selected from the group consisting of oxygen, sulfur and nitrogen (NH) as chain member(s); C₃₋₇-cycloalkyl or C₅₋₆-cycloalkenyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or represents a residue selected from the group consisting of phenyl, pyrrolyl, furanyl, thiophenyl, pyrazolyl, imidazolyl, thiazolyl, thiadiazolyl, triazolyl, oxazolyl, oxadiazolyl, isoxazolyl, pyridinyl, pyridazinyl, pyrimidinyl, pyrazinyl and pyranyl, which can respectively be bonded via a C₁₋₃-alkylene, C₂₋₃-alkenylene or C₂₋₃-alkinylene group and/or is unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅; and
R¹⁰ represents —C(═O)—OH; —C(═O)—H; —C(═O)—R¹⁵; —C(═O)—O—R¹⁶; —C(═O)—NH₂; —C(═O)—NH—R¹⁷; —C(═S)—NH—R¹⁷; —C(═O)—NR¹⁸R¹⁹; —C(═S)—NR¹⁸R¹⁹; —S(═O)—R²⁰; —S(═O)₂—R²¹; C₁₋₆-alkyl, C₂₋₆-alkenyl or C₂₋₆-alkinyl, which is respectively substituted with 1, 2, 3, 4 or 5 substituents independently selected from the group consisting of F, Cl, Br, I, —NO₂, —CN, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; 2- to 6-membered heteroalkenyl or 2- to 6-membered heteroalkinyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂ and —N(CH₃)(C₂H₅) and has 1 or 2 heteroatom(s) independently selected from the group consisting of oxygen, sulfur and nitrogen (NH) as chain member(s); C₃₋₇-cycloalkyl or C₅₋₆-cycloalkenyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or represents a residue selected from the group consisting of phenyl, pyrrolyl, furanyl, thiophenyl, pyrazolyl, imidazolyl, thiazolyl, thiadiazolyl, triazolyl, oxazolyl, oxadiazolyl, isoxazolyl, pyridinyl, pyridazinyl, pyrimidinyl, pyrazinyl and pyranyl, which can respectively be bonded via a C₁₋₃-alkylene, C₂₋₃-alkenylene or C₂₋₃-alkinylene group and/or is unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅; and
   if R¹ represents a residue selected from the group consisting of methyl, ethyl, n-propyl, isopropyl, n-butyl, isobutyl, 2-butyl, tert-butyl and cyclopropyl, then R¹⁰ can additionally represent H, unsubstituted C₁₋₆-alkyl or 2- to 6-membered heteroalkyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂ and —N(CH₃)(C₂H₅) and contains 1 or 2 heteroatom(s) independently selected from the group consisting of oxygen, sulfur and nitrogen (NH) as chain member(s); or
R⁹ and R¹⁰ together with the nitrogen atom to which they are bound form a group selected from the group consisting of imidazolidinyl, azepanyl, pyrrolyl, diazepanyl, isothioazolidinyl, (2,3)-dihydro-1H-isoindolyl, indolinyl, octahydro-1H-isoindolyl, (2,3)-dihydropyrrolyl, (2,5)-dihydropyrrolyl, 8-azaspiro[4.5]decyl, 8-azaspiro[4.5]decane-7,9-dionyl, isoindoline-1,3-dionyl, pyrrolidine-2,5-dionyl, morpholine-3,5-dionyl, 1H-pyrrole-2,5-dionyl, piperidine-2,6-dionyl, hexahydro-2H-isoindoline-1,3-dionyl, pyrrolidine-2-onyl and 1,1-dioxo-1λ⁶-isothiazolidinyl, which can be respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃, —S—C₂H₅, —NH—C(═O)—CF₃ and phenyl, wherein the phenyl residue can be unsubstituted or substituted with 1, 2, 3, 4 or 5 substituents independently selected from the group consisting of F, Cl, Br, I, —CN, —CF₃, —OH, —NH₂, —O—CF₃, —SH, —O—CH₃, —O—C₂H₅, —O—C₃H₇, methyl, ethyl, n-propyl, isopropyl, n-butyl, 2-butyl, isobutyl and tert-butyl;
   and the meaning of the remaining residues in each case is as specified above, in each case optionally in the form of one of their pure stereoisomers, in particular enantiomers or diastereomers, their racemates, or in the form of a mixture of stereoisomers, in particular the enantiomers and/or diastereomers, in any mixture ratio, or respectively in the form of corresponding salts or respectively in the form of corresponding solvates.

Substituted 4-amino quinazoline compounds corresponding to the foregoing formula I are additionally preferred in which:
R¹¹, R¹², R¹³ and R¹⁴ each independently represent H; F; Cl; Br; I; —NO₂; —CN; —NH₂; —OH; —SH; —C(═O)—OH; —C(═O)—H; —C(═O)—NH₂; —NH—R²²; —NR²³R²⁴; —O—R²⁵; —S—R²⁶; or C₁₋₆-alkyl, C₂₋₆-alkenyl or C₂₋₆-alkinyl, which is respectively unsubstituted or substituted with optionally 1, 2, 3, 4 or 5 substituents independently selected from the group consisting of F, Cl, Br, I, —NO₂, —CN, —OH, —SH and —NH₂;
   and the meaning of the remaining residues in each case is as specified above, in each case possibly in the form of one of their pure stereoisomers, in particular enantiomers or diastereomers, their racemates, or in the form of a mixture of stereoisomers, in particular the enantiomers and/or diastereomers, in any mixture ratio, or respectively in the form of corresponding salts or respectively in the form of corresponding solvates.

Substituted 4-amino quinazoline compounds corresponding to the foregoing formula I are also preferred in which:
R¹⁵ represents —C(═O)—O—R¹⁶; C₁₋₆-alkyl, C₂₋₆-alkenyl or C₂₋₆-alkinyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; 2- to 6-membered heteroalkyl, 2- to 6-membered heteroalkenyl or 2- to 6-membered heteroalkinyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃ and contains 1 or 2 heteroatom(s) independently selected from the group consisting of oxygen, sulfur and nitrogen (NH) as chain member(s); C₃₋₇-cycloalkyl, C₅₋₆-cycloalkenyl, 5- to 7-membered heterocycloalkyl or 5- to 7-membered heterocycloalkenyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅ and can optionally contain 1 or 2 heteroatom(s) independently selected from the group consisting of oxygen, sulfur and nitrogen (NH) as ring member(s); or represents a residue selected from the group consisting of phenyl, naphthyl, anthracenyl, pyrrolyl, indolyl, furanyl, benzo[b]furanyl, thiophenyl, benzo[b]thiophenyl, benzo[d]thiazolyl, pyrazolyl, imidazolyl, thiazolyl, thiadiazolyl, triazolyl, oxazolyl, oxadiazolyl, isoxazolyl, pyridinyl, pyridazinyl, pyrimidinyl, pyrazinyl, pyranyl, indazolyl, quinolinyl, isoquinolinyl and quinazolinyl, which can respectively be bonded via a C₁₋₃-alkylene, C₂₋₃-alkenylene or C₂₋₃-alkinylene group, or 2- or 3-membered heteroalkylene, which has 1 heteroatom selected from the group consisting of oxygen, sulfur and nitrogen (NH) as ring member, and/or is unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
   and the meaning of the remaining residues in each case is as specified above, in each case optionally in the form of one of their pure stereoisomers, in particular enantiomers or diastereomers, their racemates, or in the form of a mixture of stereoisomers, in particular the enantiomers and/or diastereomers, in any mixture ratio, or respectively in the form of corresponding salts or respectively in the form of corresponding solvates.

Substituted 4-amino quinazoline compounds corresponding to the foregoing formula I are additionally preferred in which:
R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², R³³, R³⁴ and R³⁵ each independently represent C₁₋₆-alkyl, C₂₋₆-alkenyl or C₂₋₆-alkinyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; C₃₋₇-cycloalkyl or C₅₋₆-cycloalkenyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or represent a residue selected from the group consisting of phenyl, pyrrolyl, furanyl, thiophenyl, pyrazolyl, imidazolyl, thiazolyl, thiadiazolyl, triazolyl, oxazolyl, oxadiazolyl, isoxazolyl, pyridinyl, pyridazinyl, pyrimidinyl, pyrazinyl and pyranyl, which can be respectively bonded via a C₁₋₃-alkylene, C₂₋₃-alkenylene or C₂₋₃-alkinylene group and/or is unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
   and the meaning of the remaining residues in each case is as specified above, in each case optionally in the form of one of their pure stereoisomers, in particular enantiomers or diastereomers, their racemates, or in the form of a mixture of stereoisomers, in particular the enantiomers and/or diastereomers, in any mixture ratio, or respectively in the form of corresponding salts or respectively in the form of corresponding solvates.

Substituted 4-amino quinazoline compounds corresponding to the foregoing formula I are particularly preferred in which T, U, V and optionally W together with two carbon atoms form a ring selected from the group consisting of:

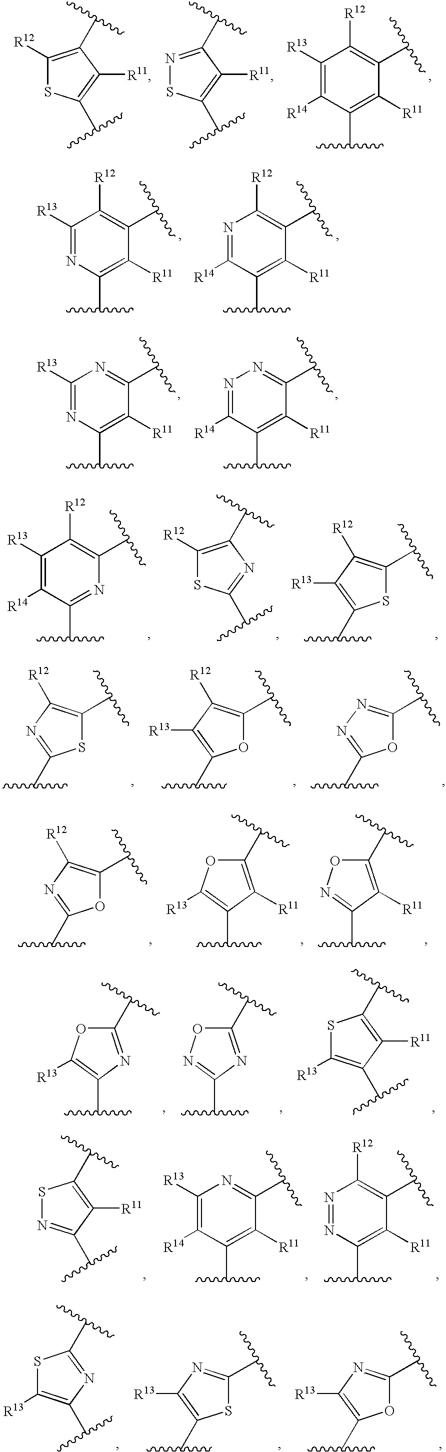

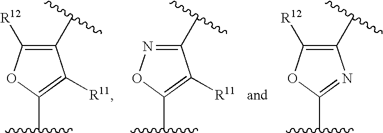

R¹ and R² each independently represent H; —C(═O)—OH; —C(═O)—H; —C(═O)—R¹⁵; —C(═O)—O—R¹⁶; —C(═O)—NH₂; —C(═O)—NH—R¹⁷; —C(═S)—NH—R¹⁷; —C(═O)—NR¹⁸R¹⁹; —C(═S)—NR¹⁸R¹⁹; —S(═O)—R²⁰; —S(═O)₂—R²¹; C₁₋₆-alkyl, C₂₋₆-alkenyl or C₂₋₆-alkinyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; 2- to 6-membered heteroalkyl, 2- to 6-membered heteroalkenyl or 2- to 6-membered heteroalkinyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃ and contains 1 or 2 heteroatom(s) independently selected from the group consisting of oxygen, sulfur and nitrogen (NH) as chain member(s); C₃₋₇-cycloalkyl, C₅₋₆-cycloalkenyl, 5- to 7-membered heterocycloalkyl or 5- to 7-membered heterocycloalkenyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅ and can optionally contain 1 or 2 heteroatom(s) selected from the group consisting of oxygen, sulfur and nitrogen (NH) as ring member(s); or denote a residue selected from the group consisting of phenyl, naphthyl and anthracenyl, which can respectively be bonded via a C₁₋₃-alkylene, C₂₋₃-alkenylene or C₂₋₃-alkinylene group and/or is unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, piperazinyl, pyrrolidinyl, piperidinyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅; or
R¹ and R² together with the nitrogen atom to which they are bound form a 5- to 7-membered heterocycloalkyl or 5- to 7-membered heterocycloalkenyl group, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅, and may contain 1 or 2 further heteroatom(s) independently selected from the group consisting of oxygen, sulfur and nitrogen (NH) as ring member(s);
R³ represents H; F; Cl; Br; I; —NO₂; —CN; —NH₂; —OH; —SH; —C(═O)—OH; —C(═O)—H; —C(═O)—R¹⁵; —C(═O)—O—R¹⁶; —C(═O)—NH₂; —C(═O)—NH—R¹⁷; —C(═S)—NH—R¹⁷; —C(═O)—NR¹⁸R¹⁹; —C(═S)—NR¹⁸R¹⁹; —NH—R²²; —NR²³R²⁴; —O—R²⁵; —S—R²⁶; C₁₋₆-alkyl, C₂₋₆-alkenyl or C₂₋₆-alkinyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃ and —C(═O)—O—C₂H₅; C₃₋₇-cycloalkyl, C₅₋₆-cycloalkenyl, 5- to 7-membered heterocycloalkyl or 5- to 7-membered heterocycloalkenyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅, and can possibly have 1 or 2 heteroatom(s) independently selected from the group consisting of oxygen, sulfur and nitrogen (NH) as ring member(s); or represents a residue selected from the group consisting of phenyl, pyrrolyl, furanyl, thiophenyl, pyrazolyl, imidazolyl, thiazolyl, thiadiazolyl, triazolyl, oxazolyl, oxadiazolyl, isoxazolyl, pyridinyl, pyridazinyl, pyrimidinyl, pyrazinyl and pyranyl, which can respectively be bonded via a C₁₋₃-alkylene, C₂₋₃-alkenylene or C₂₋₃-alkinylene group and/or be unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂Hs, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
R⁴, R⁵ and R⁶ each independently represent H; F; Cl; Br; I; —NO₂; —CN; —NH₂; —OH; —SH; —C(═O)—OH; —C(═O)—H; —C(═O)—NH₂; —NH—R²²; —NR²³R²⁴; —O—R²⁵; —S—R²⁶; or C₁₋₆-alkyl, C₂₋₆-alkenyl or C₂₋₆-alkinyl, which is respectively unsubstituted or substituted with optionally 1, 2, 3, 4 or 5 substituents independently selected from the group consisting of F, Cl, Br, I, —NO₂, —CN, —OH, —SH and —NH₂;
R⁷ and R⁸ each independently represent H; F; Cl; Br; I; —NO₂; —CN; —NH₂; —OH; —SH; —C(═O)—OH; —C(═O)—H; —C(═O)—NH₂; —NH—R²²; —NR²³R²⁴; —O—R²⁵; —S—R²⁶; or C₁₋₆-alkyl, C₂₋₆-alkenyl or C₂₋₆-alkinyl, which is respectively unsubstituted or substituted with optionally 1, 2, 3, 4 or 5 substituents independently selected from the group consisting of F, Cl, Br, I, —NO₂, —CN, —OH, —SH and —NH₂;
R⁹ represents H; —C(═O)—OH; —C(═O)—H; —C(═O)—R¹⁵; —C(═O)—O—R¹⁶; —C(═O)—NH₂; —C(═O)—NH—R¹⁷; —C(═S)—NH—R¹⁷; —C(═O)—NR¹⁸R¹⁹; —C(═S)—NR¹⁸R¹⁹; —S(═O)—R²⁰; —S(═O)₂—R²¹; C₁₋₆-alkyl, C₂₋₆-alkenyl or C₂₋₆-alkinyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; 2- to 6-membered heteroalkyl, 2- to 6-membered heteroalkenyl or 2- to 6-membered heteroalkinyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂ and —N(CH₃)(C₂H₅) and contains 1 or 2 heteroatom(s) independently selected from the group consisting of oxygen, sulfur and nitrogen (NH) as chain member(s); C₃₋₇-cycloalkyl or C₅₋₆-cycloalkenyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or represents a residue selected from the group consisting of phenyl, pyrrolyl, furanyl, thiophenyl, pyrazolyl, imidazolyl, thiazolyl, thiadiazolyl, triazolyl, oxazolyl, oxadiazolyl, isoxazolyl, pyridinyl, pyridazinyl, pyrimidinyl, pyrazinyl and pyranyl, which can respectively be bonded via a C₁₋₃-alkylene, C₂₋₃-alkenylene or C₂₋₃-alkinylene group and/or is unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
R¹⁰ represents —C(═O)—OH; —C(═O)—H; —C(═O)—R¹⁵; —C(═O)—O—R¹⁶; —C(═O)—NH₂; —C(═O)—NH—R¹⁷; —C(═S)—NH—R¹⁷; —C(═O)—NR¹⁸R¹⁹; —C(═S)—NR¹⁸R¹⁹; —S(═O)—R²⁰; —S(═O)₂—R²¹; C₁₋₆-alkyl, C₂₋₆-alkenyl or C₂₋₆-alkinyl, which is respectively substituted with 1, 2, 3, 4 or 5 substituents independently selected from the group consisting of F, Cl, Br, I, —NO₂, —CN, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; 2- to 6-membered heteroalkenyl or 2- to 6-membered heteroalkinyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂ and —N(CH₃)(C₂H₅) and has 1 or 2 heteroatom(s) independently selected from the group consisting of oxygen, sulfur and nitrogen (NH) as chain member(s); C₃₋₇-cycloalkyl or C₅₋₆-cycloalkenyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or represents a residue selected from the group consisting of phenyl, pyrrolyl, furanyl, thiophenyl, pyrazolyl, imidazolyl, thiazolyl, thiadiazolyl, triazolyl, oxazolyl, oxadiazolyl, isoxazolyl, pyridinyl, pyridazinyl, pyrimidinyl, pyrazinyl and pyranyl, which can respectively be bonded via a C₁₋₃-alkylene, C₂₋₃-alkenylene or C₂₋₃-alkinylene group and/or is unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
   and if R¹ represents a residue selected from the group consisting of methyl, ethyl, n-propyl, isopropyl, n-butyl, isobutyl, 2-butyl, tert-butyl and cyclopropyl, then R¹⁰ can additionally represent H, unsubstituted C₁₋₆-alkyl or 2- to 6-membered heteroalkyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂ and —N(CH₃)(C₂H₅) and contains 1 or 2 heteroatom(s) independently selected from the group consisting of oxygen, sulfur and nitrogen (NH) as chain member(s); or
R⁹ and R¹⁰ together with the nitrogen atom linking them represent a residue selected from the group consisting of imidazolidinyl, azepanyl, pyrrolyl, diazepanyl, isothioazolidinyl, (2,3)-dihydro-1H-isoindolyl, indolinyl, octahydro-1H-isoindolyl, (2,3)-dihydropyrrolyl, (2,5)-dihydropyrrolyl, 8-azaspiro[4.5]decyl, 8-azaspiro[4.5]decane-7,9-dionyl, isoindoline-1,3-dionyl, pyrrolidine-2,5-dionyl, morpholine-3,5-dionyl, 1H-pyrrole-2,5-dionyl, piperidine-2,6-dionyl, hexahydro-2H-isoindoline-1,3-dionyl, pyrrolidine-2-onyl and 1,1-dioxo-1λ⁶-isothiazolidinyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃, —S—C₂H₅, —NH—C(═O)—CF₃ and phenyl;
R¹¹, R¹², R¹³ and R¹⁴ each independently represent H; F; Cl; Br; I; —NO₂; —CN; —NH₂; —OH; —SH; —C(═O)—OH; —C(═O)—H; —C(═O)—NH₂; —NH—R²²; —NR²³R²⁴; —O—R²⁵; —S—R²⁶; or C₁₋₆-alkyl, C₂₋₆-alkenyl or C₂₋₆-alkinyl, which is respectively unsubstituted or substituted with optionally 1, 2, 3, 4 or 5 substituents independently selected from the group consisting of F, Cl, Br, I, —NO₂, —CN, —OH, —SH and —NH₂;
R¹⁵ represents —C(═O)—O—R¹⁶; C₁₋₆-alkyl, C₂₋₆-alkenyl or C₂₋₆-alkinyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; 2- to 6-membered heteroalkyl, 2- to 6-membered heteroalkenyl or 2- to 6-membered heteroalkinyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃ and contains 1 or 2 heteroatom(s) independently selected from the group consisting of oxygen, sulfur and nitrogen (NH) as chain member(s); C₃₋₇-cycloalkyl, C₅₋₆-cycloalkenyl, 5- to 7-membered heterocycloalkyl or 5- to 7-membered heterocycloalkenyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅ and can possibly have 1 or 2 heteroatom(s) independently selected from the group consisting of oxygen, sulfur and nitrogen (NH) as ring member(s); or represents a residue selected from the group consisting of phenyl, naphthyl, anthracenyl, pyrrolyl, indolyl, furanyl, benzo[b]furanyl, thiophenyl, benzo[b]thiophenyl, benzo[d]thiazolyl, pyrazolyl, imidazolyl, thiazolyl, thiadiazolyl, triazolyl, oxazolyl, oxadiazolyl, isoxazolyl, pyridinyl, pyridazinyl, pyrimidinyl, pyrazinyl, pyranyl, indazolyl, quinolinyl, isoquinolinyl and quinazolinyl, which can respectively be bonded via a C₁₋₃-alkylene-, C₂₋₃-alkenylene- or C₂₋₃-alkinylene group or 2- or 3-membered heteroalkylene; which has 1 heteroatom selected from the group consisting of oxygen, sulfur and nitrogen (NH) as chain member, and/or is unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
R¹⁶, R¹⁷, R¹⁸, R¹⁹, R²⁰, R²¹, R²², R²³, R²⁴, R²⁵ and R²⁶ each independently represent C₁₋₆-alkyl, C₂₋₆-alkenyl or C₂₋₆-alkinyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; C₃₋₇-cycloalkyl or C₅₋₆-cycloalkenyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or represent a residue selected from the group consisting of phenyl, pyrrolyl, furanyl, thiophenyl, pyrazolyl, imidazolyl, thiazolyl, thiadiazolyl, triazolyl, oxazolyl, oxadiazolyl, isoxazolyl, pyridinyl, pyridazinyl, pyrimidinyl, pyrazinyl and pyranyl, which can be respectively bonded via a C₁₋₃-alkylene, C₂₋₃-alkenylene or C₂₋₃-alkinylene group and/or is unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
   in each case optionally in the form of one of their pure stereoisomers, in particular enantiomers or diastereomers, their racemates, or in the form of a mixture of stereoisomers, in particular the enantiomers and/or diastereomers, in any mixture ratio, or respectively in the form of corresponding salts or respectively in the form of corresponding solvates.

Substituted 4-amino quinazoline compounds corresponding to the foregoing formula I are also particularly preferred in which T, U, V and optionally W together with two carbon atoms form a ring selected from the group consisting of from the group consisting of:

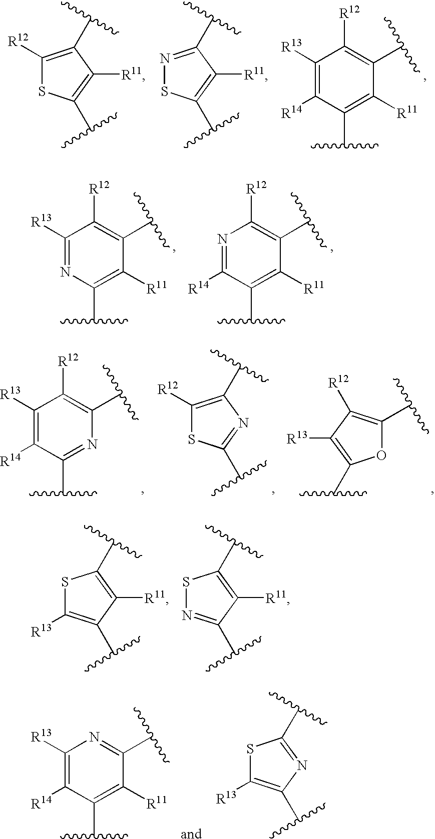

R¹ represents —C(═O)—R¹⁵; an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; a residue selected from the group consisting of cyclopropyl, cyclobutyl, cyclopentyl, cyclohexyl, cycloheptyl, cyclopentenyl and cyclohexenyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or a residue selected from the group consisting of phenyl, naphthyl, benzyl and phenethyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
R² represents H; —C(═O)—R¹⁵; or an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; or
R¹ and R² together with the nitrogen atom to which they are bound form a residue selected from the group consisting of piperazinyl, piperidinyl, morpholinyl, thiomorpholinyl, pyrrolidinyl, azepanyl and diazepanyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —NO₂, —CF₃, —O—CF₃, —S—CF₃, —SH, —S—CH₃ and —S—C₂H₅;
R³ represents H; F; Cl; Br; I; —NO₂; —CN; —NH₂; —OH; —SH; —NH—R²²; —NR²³R²⁴; —O—R²⁵; —S—R²⁶; an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃ and —C(═O)—O—C₂H₅; for a residue selected from the group consisting of morpholinyl, piperazinyl, piperidinyl, pyrrolidinyl, azepanyl, diazepanyl, thiomorpholinyl, cyclopropyl, cyclobutyl, cyclopentyl, cyclohexyl, cycloheptyl, cyclopentenyl and cyclohexenyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —NO₂, —CF₃, —O—CF₃, —S—CF₃, —SH, —S—CH₃ and —S—C₂H₅; or represents a residue selected from the group consisting of phenyl, benzyl, phenethyl, pyridinyl, pyridazinyl, pyrimidinyl and pyrazinyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
R⁴, R⁵ and R⁶ each independently represent H; F; Cl; Br; I; —NO₂; —CN; —O—R²⁵; —S—R²⁶; or an alkyl residue selected from the group consisting of methyl, ethyl, n-propyl, isopropyl, n-butyl, 2-butyl, isobutyl and tert-butyl, which is respectively unsubstituted or substituted with optionally 1, 2, 3, 4 or 5 substituents independently selected from the group consisting of F, Cl, Br, I, —NO₂ and —CN;
R⁷ and R⁸ each independently represent H; F; Cl; Br; I; —NO₂; —CN; —O—R²⁵; —S—R²⁶; or an alkyl residue selected from the group consisting of methyl, ethyl, n-propyl, isopropyl, n-butyl, 2-butyl, isobutyl and tert-butyl, which is respectively unsubstituted or substituted with optionally 1, 2, 3, 4 or 5 substituents independently selected from the group consisting of F, Cl, Br, I, —NO₂ and —CN;
R⁹ represents H; —C(═O)—R¹⁵; —C(═O)—O—R¹⁶; —S(═O)₂—R²¹; an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; a heteroalkyl residue selected from the group consisting of —CH₂—O—CH₃, —CH₂—O—C₂H₅, —CH₂—O—CH(CH₃)₂, —CH₂—O—C(CH₃)₃, —CH₂—CH₂—O—CH₃, —CH₂—CH₂—O—C₂H₅, —CH₂—CH₂—O—CH(CH₃)₂, —CH₂—CH₂—O—C(CH₃)₃, —CH₂—O—CH₂—O—C₂H₅ and —CH₂—O—CH₂—O—CH(CH₃)₂; a residue selected from the group consisting of cyclopropyl, cyclobutyl, cyclopentyl, cyclohexyl, cycloheptyl, cyclopentenyl and cyclohexenyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or a residue selected from the group consisting of pyridinyl, thiazolyl, pyridazinyl, pyrimidinyl, pyrazinyl, furanyl, —(CH₂)-furanyl, —(CH₂)—(CH₂)-furanyl, thiophenyl, phenyl, naphthyl, benzyl and phenethyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
R¹⁰ represents —C(═O)—R¹⁵; —C(═O)—O—R¹⁶; —C(═O)—NH—R¹⁷; —C(═S)—NH—R¹⁷; —C(═O)—NR¹⁸R¹⁹; —C(═S)—NR¹⁸R¹⁹; —S(═O)—R²⁰; —S(═O)₂—R²¹; or an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which is respectively substituted with optionally 1, 2, 3, 4 or 5 substituents independently selected from the group consisting of F, Cl, Br, I, —NO₂, —CN, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃;
   and if R¹ represents a residue selected from the group consisting of methyl, ethyl, n-propyl, isopropyl, n-butyl, isobutyl, 2-butyl, tert-butyl and cyclopropyl, then R¹⁰ can additionally represent H or an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which can respectively be unsubstituted or substituted with 1, 2 or 3 substituents independently selected from the group consisting of —O—CH₃, —O—C₂H₅, —O—CH₂—C₂—CH₃, —O—CH(CH₃)₂ and —O—C(CH₃)₃; or
   R⁹ and R¹⁰ together with the nitrogen atom to which they are bound form a residue selected from the group consisting of imidazolidinyl, azepanyl, pyrrolyl, diazepanyl, isothioazolidinyl, (2,3)-dihydro-1H-isoindolyl, indolinyl, octahydro-1H-isoindolyl, (2,3)-dihydropyrrolyl, (2,5)-dihydropyrrolyl, 8-azaspiro[4.5]decyl, 8-azaspiro[4.5]decane-7,9-dionyl, isoindoline-1,3-dionyl, pyrrolidine-2,5-dionyl, morpholine-3,5-dionyl, 1H-pyrrole-2,5-dionyl, piperidine-2,6-dionyl, hexahydro-2H-isoindoline-1,3-dionyl, pyrrolidine-2-onyl and 1,1-dioxo-1λ⁶-isothiazolidinyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, thioxo, —O—CH₃, —O—C₂H₅, —O—C₃H₇, —NH—C(═O)—CF₃ and phenyl;
R¹¹, R¹², R¹³ and R¹⁴ each independently represent H; F; Cl; Br; I; —NO₂; —CN; —O—R²⁵; —S—R²⁶; or for an alkyl residue selected from the group consisting of methyl, ethyl, n-propyl, isopropyl, n-butyl, 2-butyl, isobutyl and tert-butyl, which is respectively unsubstituted or substituted with optionally 1, 2, 3, 4 or 5 substituents independently selected from the group consisting of F, Cl, Br, I, —NO₂, —CN, —OH, —SH and —NH₂;
R¹⁵ represents —C(═O)—O—R¹⁶; an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; an alkenyl residue selected from the group consisting of ethenyl, 1-propenyl, 2-propenyl, 1-butenyl, 2-butenyl, 3-butenyl and —CH═C(CH₃)₂; for a heteroalkyl residue selected from the group consisting of —CH₂—O—CH₃, —CH₂—O—C₂H₅, —CH₂—O—CH(CH₃)₂, —CH₂—O—C(CH₃)₃, —CH₂—CH₂—O—CH₃, —CH₂—CH₂—O—C₂H₅, —CH₂—CH₂—O—CH(CH₃)₂, —CH₂—CH₂—O—C(CH₃)₃, —CH₂—O—CH₂—O—C₂H₅ and —CH₂—O—CH₂—O—CH(CH₃)₂; a residue selected from the group consisting of cyclopropyl, cyclobutyl, cyclopentyl, cyclohexyl, cycloheptyl, cyclopentenyl and cyclohexenyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or represents a residue selected from the group consisting of phenyl, naphthyl, furanyl, thiophenyl, oxazolyl, oxadiazolyl, isoxazolyl, pyridinyl, pyridazinyl, pyrimidinyl, pyrazinyl, quinolinyl, isoquinolinyl and quinazolinyl, which can respectively be bonded via a —(CH₂)—, —(CH₂)—(CH₂)—, —(CH₂)—(CH₂)—(CH₂)—, —(CH═CH)— or —(CH₂)—O— group and/or is unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
R¹⁶, R¹⁷, R¹⁸, R¹⁹, R²⁰, R²¹, R²², R²³, R²⁴, R²⁵ and R²⁶ each independently represent an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which is respectively unsubstituted or substituted with optionally 1, 2, 3, 4 or 5 substituents independently selected from the group consisting of F, Cl, Br, I, —NO₂, —CN, —OH, —SH, —NH₂, —C(═O)—OH, —C(═O)—O—CH₃, —C(═O)—O—C₂H₅ and —C(═O)—O—C(CH₃)₃; a residue selected from the group consisting of cyclopropyl, cyclobutyl, cyclopentyl, cyclohexyl, cycloheptyl, cyclopentenyl and cyclohexenyl, which is respectively unsubstituted; or a residue selected from the group consisting of phenyl and benzyl, which is respectively unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NO₂, —CF₃, —O—CF₃, —S—CF₃, —SH, —C(═O)—O—CH₃ and —C(═O)—O—C₂H₅;
   in each case optionally in the form of one of their pure stereoisomers, in particular enantiomers or diastereomers, their racemates, or in the form of a mixture of stereoisomers, in particular the enantiomers and/or diastereomers, in any mixture ratio, or respectively in the form of corresponding salts or respectively in the form of corresponding solvates.

Most particularly preferred are substituted 4-amino-quinazoline compounds corresponding to the foregoing formula I in which T, U, V and optionally W together with two carbon atoms form a ring selected from the group consisting of:

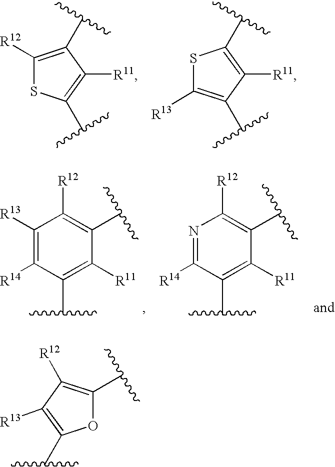

R¹ represents a residue selected from the group consisting of methyl, ethyl, n-propyl, isopropyl, n-butyl, 2-butyl, isobutyl, tert-butyl, n-pentyl, sec-pentyl, neo-pentyl and n-hexyl, —(CH₂)—C(═O)—OH and —(CH₂)—(CH₂)—C(═O)—OH; for a residue selected from the group consisting of cyclopropyl, cyclobutyl, cyclopentyl, cyclohexyl, cycloheptyl, cyclopentenyl and cyclohexenyl; or for a residue selected from the group consisting of phenyl, benzyl and phenethyl;
R² represents H; —C(═O)—CH₃; —C(═O)—C₂H₅; or for an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl; or
R¹ and R² together with the nitrogen atom to which they are bound form a residue selected from the group consisting of piperazinyl, piperidinyl, morpholinyl, thiomorpholinyl, pyrrolidinyl, azepanyl and diazepanyl;
R³ represents H; F; Cl; Br; —NH₂; —NH—R²²; —NR²³R²⁴; an alkyl residue selected from the group consisting of methyl, ethyl, n-propyl, isopropyl, n-butyl, 2-butyl, isobutyl, tert-butyl, n-pentyl, sec-pentyl, neo-pentyl, n-hexyl, —CF₃, —CHF₂, —CH₂F and —CF₂—CF₃; a residue selected from the group consisting of morpholinyl, piperazinyl, piperidinyl, pyrrolidinyl, azepanyl, diazepanyl and thiomorpholinyl; or a residue selected from the group consisting of phenyl, benzyl, phenethyl and pyridinyl;
R⁴, R⁵ and R⁶ each respectively represent H, F, Cl, —CF₃, methyl, ethyl, —CN or —O—CH₃;
R⁷ and R⁸ each independently represent H or for an alkyl residue selected from the group consisting of methyl, ethyl, n-propyl, isopropyl, n-butyl, 2-butyl, isobutyl and tert-butyl;
R⁹ represents H; —C(═O)—O—R¹⁶; an alkyl residue selected from the group consisting of methyl, ethyl, n-propyl, isopropyl, n-butyl, 2-butyl, isobutyl, tert-butyl, n-pentyl, sec-pentyl, neo-pentyl, n-hexyl, —(CH₂)—(CH₂)—NH—C(═O)—O—C(CH₃)₃, —(CH₂)—NH—C(═O)—O—C(CH₃)₃, —(CH₂)—CN, —(CH₂)—(CH₂)—CN, —(CH₂)—Cl, —(CH₂)—(CH₂)—Cl, —(CH₂)—NH₂, —(CH₂)—(CH₂)—NH₂, —(CH₂)—C(═O)—O—CH₃, —(CH₂)—(CH₂)—C(═O)—O—CH₃ and —(CH₂)—C(═O)—O—C₂H₅; for a heteroalkyl residue selected from the group consisting of —CH₂—O—CH₃, —CH₂—O—C₂H₅, —CH₂—O—CH(CH₃)₂, —CH₂—O—C(CH₃)₃, —CH₂—CH₂—O—CH₃ and —CH₂—CH₂—O—C₂H₅; a residue selected from the group consisting of cyclopropyl, cyclobutyl, cyclopentyl, cyclohexyl, cycloheptyl, cyclopentenyl and cyclohexenyl; or a residue selected from the group consisting of pyridinyl, thiazolyl, —(CH₂)-furanyl, —(CH₂)—(CH₂)— furanyl, thiophenyl, phenyl, benzyl and phenethyl, which is respectively unsubstituted or is substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —O—CH₃, —O—C₂H₅ and —O—C₃H₇;
R¹⁰ represents —C(═O)—R¹⁵; —C(═O)—NH—R¹⁷; —C(═S)—NH—R¹⁷; —C(═O)—NR¹⁸R¹⁹; —C(═S)—NR¹⁸R¹⁹ or —S(═O)₂—R²¹;
   and if R¹ represents a residue selected from the group consisting of methyl, ethyl, n-propyl, isopropyl, n-butyl, isobutyl, 2-butyl, tert-butyl and cyclopropyl, then R¹⁰ additionally may represent H or an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which can respectively be unsubstituted or substituted with 1, 2 or 3 substituents independently selected from the group consisting of —O—CH₃, —O—C₂H₅, —O—CH₂—C₂—CH₃, —O—CH(CH₃)₂ and —O—C(CH₃)₃; or
R⁹ and R¹⁰ together with the nitrogen atom linking them represent a residue selected from the group consisting of:

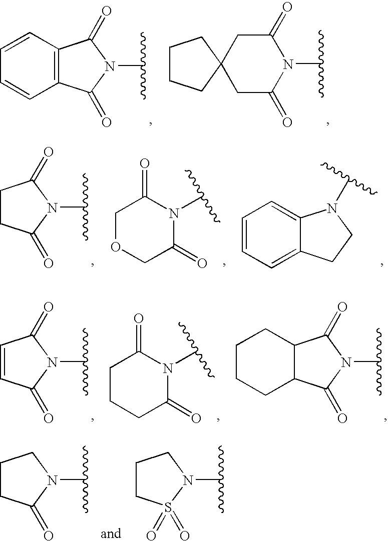

which is respectively unsubstituted or substituted with optionally 1, 2 or 3 substituents independently selected from the group consisting of methyl, ethyl, n-propyl, isopropyl, n-butyl, 2-butyl, isobutyl, tert-butyl, —NH—C(═O)—CF₃ and phenyl;
R¹¹ represents H; F; Cl; Br; —O—CH₃; —O—C₂H₅; —O—CH(CH₃)₂; —O—C(CH₃)₃; methyl; ethyl; n-propyl or isopropyl;
R¹² represents H; F; Cl; Br; —O—CH₃; —O—C₂H₅; —O—CH(CH₃)₂; —O—C(CH₃)₃; methyl; ethyl; n-propyl or isopropyl;
R¹³ represents H; F; Cl; Br; —O—CH₃; —O—C₂H₅; —O—CH(CH₃)₂; —O—C(CH₃)₃; methyl; ethyl; n-propyl or isopropyl;
R¹⁴ represents H; F; Cl; Br; —O—CH₃; —O—C₂H₅; —O—CH(CH₃)₂; —O—C(CH₃)₃; methyl; ethyl; n-propyl or isopropyl;
R¹⁵ represents —C(═O)—O—R¹⁶; an alkyl group from the group consisting of methyl, ethyl, n-propyl, isopropyl, n-butyl, 2-butyl, isobutyl, tert-butyl, n-pentyl, sec-pentyl, neo-pentyl, n-hexyl, —CF₃, —(CH₂)—F, —CHF₂, —(CH₂)—Cl, —(CH₂)—CF₃, —CF₂—CF₃, —(CH₂)—CN, —(CH₂)—N(CH₃)₂, —(CH₂)—(CH₂)—CN, —(CH₂)—(CH₂)—N(CH₃)₂, —(CH₂)—C(═O)—O—CH₃, —(CH₂)—C(═O)—OH, —(CH₂)—(CH₂)—C(═O)—O—CH₃ and —(CH₂)—(CH₂)—C(═O)—OH; an alkenyl residue selected from the group consisting of ethenyl, 1-propenyl, 2-propenyl, 1-butenyl, 2-butenyl, 3-butenyl and —CH═C(CH₃)₂; a heteroalkyl residue selected from the group consisting of —CH₂—O—CH₃, —CH₂—O—C₂H₅, —CH₂—O—CH(CH₃)₂, —CH₂—O—C(CH₃)₃, —CH₂—CH₂—O—CH₃ and —CH₂—CH₂—O—C₂H₅; a residue selected from the group consisting of cyclopropyl, cyclobutyl, cyclopentyl, cyclohexyl, cycloheptyl, cyclopentenyl and cyclohexenyl; a residue selected from the group consisting of phenyl and pyridinyl, which is respectively bonded via a —(CH₂)—, —(CH₂)—(CH₂)—, —(CH₂)—(CH₂)—(CH₂)—, —(CH═CH)— or —(CH₂)—O— group and/or is unsubstituted or substituted with optionally 1, 2, 3, 4 or 5 substituents independently selected from the group consisting of F, Cl, Br, I, —C(═O)—OH, —C(═O)—O—CH₃ and —C(═O)—O—C₂H₅; or represents a residue selected from the group consisting of phenyl, furanyl, thiophenyl, oxazolyl, isoxazolyl, pyridinyl, pyrazinyl, quinolinyl and isoquinolinyl, which is respectively substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NO₂, —CF₃, —O—CF₃ and —S—CF₃;
R¹⁶ represents an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl;
R¹⁷, R¹⁸ and R¹⁹ each represent an alkyl residue independently selected from the group consisting of methyl, ethyl, n-propyl, isopropyl, n-butyl, 2-butyl, isobutyl, tert-butyl, n-pentyl, sec-pentyl, neo-pentyl and n-hexyl;
R²¹ represents an alkyl residue selected from the group consisting of methyl, ethyl, n-propyl, isopropyl, n-butyl, 2-butyl, isobutyl, tert-butyl, n-pentyl, sec-pentyl, neo-pentyl, n-hexyl, —CF₃, —CHF₂, —CH₂F, —CF₂—CF₃, —(CH₂)—CF₃, —(CH₂)—Cl, —(CH₂)—(CH₂)—Cl, —(CH₂)—(CH₂)—(CH₂)—Cl and —(CH₂)—(CH₂)—(CH₂)—(CH₂)—Cl; or a residue selected from the group consisting of phenyl and benzyl, which is respectively unsubstituted or substituted with 1, 2, 3, 4 or 5 substituents independently selected from the group consisting of F, Cl, Br and —CF₃; and
R²², R²³ and R²⁴ each independently represent an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl; a residue selected from the group consisting of cyclopropyl, cyclobutyl, cyclopentyl, cyclohexyl, cycloheptyl, cyclopentenyl and cyclohexenyl; or a residue selected from the group consisting of phenyl and benzyl;
   in each case optionally in the form of one of their pure stereoisomers, in particular enantiomers or diastereomers, their racemates, or in the form of a mixture of stereoisomers, in particular the enantiomers and/or diastereomers, in any mixture ratio, or respectively in the form of corresponding salts or respectively in the form of corresponding solvates.

In a further preferred embodiment the present invention relates to substituted 4-amino-quinazoline compounds corresponding to the foregoing formula I in which T, U, V and optionally W together with two carbon atoms form a ring selected from the group consisting of:

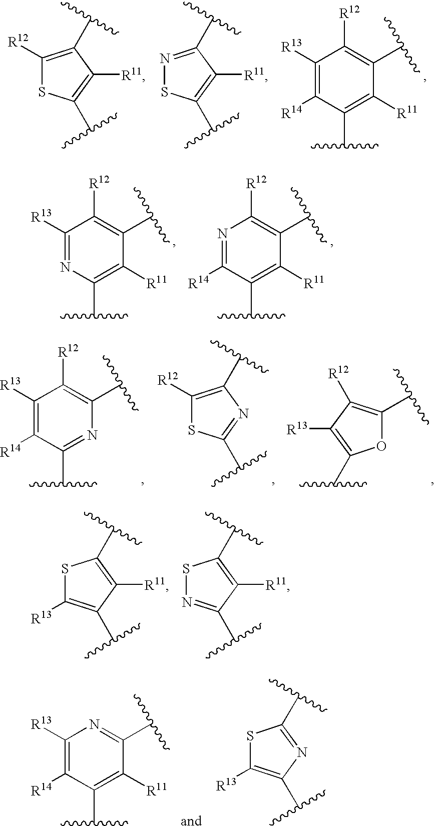

R¹ represents —C(═O)—R¹⁵; an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which is respectively unsubstituted or substituted with optionally 1, 2, 3, 4 or 5 substituents independently selected from the group consisting of F, Cl, Br, I, —NO₂, —CN, —OH, —OCH₃, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; for a residue selected from the group consisting of cyclopropyl, cyclobutyl, cyclopentyl, cyclohexyl, cycloheptyl, cyclopentenyl and cyclohexenyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or represents a residue selected from the group consisting of phenyl, naphthyl, benzyl and phenethyl, which is respectively unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
R² represents H; —C(═O)—R¹⁵; or an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; or
R¹ and R² together with the nitrogen atom to which they are bound form a residue selected from the group consisting of piperazinyl, piperidinyl, morpholinyl, thiomorpholinyl, pyrrolidinyl, azepanyl and diazepanyl, which is respectively unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —NO₂, —CF₃, —O—CF₃, —S—CF₃, —SH, —S—CH₃ and —S—C₂H₅;
R³ represents H; F; Cl; Br; I; —NO₂; —CN; —NH₂; —OH; —SH; —NH—R²²; —NR²³R²⁴; —O—R²⁵; —S—R²⁶; an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃ and —C(═O)—O—C₂H₅; for a residue selected from the group consisting of morpholinyl, piperazinyl, piperidinyl, pyrrolidinyl, azepanyl, diazepanyl, thiomorpholinyl, cyclopropyl, cyclobutyl, cyclopentyl, cyclohexyl, cycloheptyl, cyclopentenyl and cyclohexenyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —NO₂, —CF₃, —O—CF₃, —S—CF₃, —SH, —S—CH₃ and —S—C₂H₅; or represents a residue selected from the group consisting of phenyl, benzyl, phenethyl, pyridinyl, pyridazinyl, pyrimidinyl and pyrazinyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
R⁴, R⁵ and R⁶ each independently represent H; F; Cl; Br; I; —NO₂; —CN; —O—R²⁵; —S—R²⁶; or an alkyl residue selected from the group consisting of methyl, ethyl, n-propyl, isopropyl, n-butyl, 2-butyl, isobutyl and tert-butyl, which is respectively unsubstituted or substituted with optionally 1, 2, 3, 4 or 5 substituents independently selected from the group consisting of F, Cl, Br, I, —NO₂ and —CN;
R⁷ and R⁸ each independently represent H; F; Cl; Br; I; —NO₂; —CN; —O—R²⁵; —S—R²⁶; or an alkyl residue selected from the group consisting of methyl, ethyl, n-propyl, isopropyl, n-butyl, 2-butyl, isobutyl and tert-butyl, which is respectively unsubstituted or substituted with optionally 1, 2, 3, 4 or 5 substituents independently selected from the group consisting of F, Cl, Br, I, —NO₂ and —CN;
R⁹ represents H; —C(═O)—R¹⁵; —C(═O)—O—R¹⁶; —S(═O)₂—R²¹; an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; for a —CH₂—CH═CH₂ or a —CH═CH—CH₃ residue, for a heteroalkyl residue selected from the group consisting of —CH₂—O—CH₃, —CH₂—O—C₂H₅, —CH₂—O—CH(CH₃)₂, —CH₂—O—C(CH₃)₃, —CH₂—CH₂—O—CH₃, —CH₂—CH₂—O—C₂H₅, —CH₂—CH₂—O—CH(CH₃)₂, —CH₂—CH₂—O—C(CH₃)₃, —CH₂—O—CH₂—O—C₂H₅ and —CH₂—O—CH₂—O—CH(CH₃)₂; a residue selected from the group consisting of cyclopropyl, cyclobutyl, cyclopentyl, cyclohexyl, cycloheptyl, cyclopentenyl and cyclohexenyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or a residue selected from the group phenyl, benzyl, phenethyl, pyridinyl, —(CH₂)-pyridinyl, thiazolyl, pyridazinyl, pyrimidinyl, pyrazinyl, furanyl, —(CH₂)-furanyl, —(CH₂)—(CH₂)-furanyl, thiophenyl, phenyl, naphthyl, benzyl and phenethyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
R¹⁰ represents —C(═O)—R¹⁵; —C(═O)—O—R¹⁶; —C(═O)—NH—R¹⁷; —C(═S)—NH—R¹⁷; —C(═O)—NR¹⁸R¹⁹; —C(═S)—NR¹⁸R¹⁹; —S(═O)—R²⁰; —S(═O)₂—R²¹; thiazolyl, or for an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which is respectively substituted with 1, 2, 3, 4 or 5 substituents independently selected from the group consisting of F, Cl, Br, I, —NO₂, —CN, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃;
   and if R¹ represents a residue selected from the group consisting of methyl, ethyl, n-propyl, isopropyl, n-butyl, isobutyl, 2-butyl, tert-butyl and cyclopropyl, then R¹⁰ can additionally denote H or an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which can respectively be unsubstituted or substituted with 1, 2 or 3 substituents independently selected from the group consisting of —O—CH₃, —O—C₂H₅, —O—CH₂—C₂—CH₃, —O—CH(CH₃)₂ and —O—C(CH₃)₃; or
R⁹ and R¹⁰ together with the nitrogen atom to which they are bound form a residue selected from the group consisting of

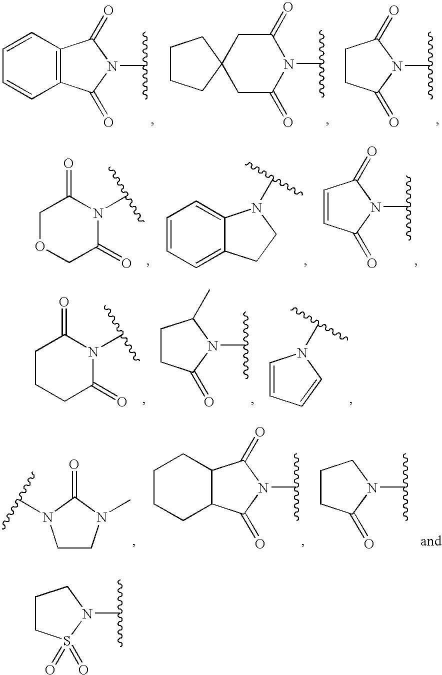

which is respectively unsubstituted or substituted with optionally 1, 2 or 3 substituents independently selected from the group consisting of methyl, ethyl, n-propyl, isopropyl, n-butyl, 2-butyl, isobutyl, tert-butyl, —NH—C(═O)—CF₃ and phenyl;
R¹¹, R¹², R¹³ and R¹⁴ independently of one another respectively represent H; F; Cl; Br; I; —NO₂; —CN; —O—R²⁵; —S—R²⁶; or for an alkyl residue selected from the group consisting of methyl, ethyl, n-propyl, isopropyl, n-butyl, 2-butyl, isobutyl and tert-butyl, which is respectively unsubstituted or substituted with optionally 1, 2, 3, 4 or 5 substituents independently selected from the group consisting of F, Cl, Br, I, —NO₂, —CN, —OH, —SH and —NH₂;
R¹⁵ represents —C(═O)—O—R¹⁶; an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; for an alkenyl residue selected from the group consisting of ethenyl, 1-propenyl, 2-propenyl, 1-butenyl, 2-butenyl, 3-butenyl and —CH═C(CH₃)₂; for a heteroalkyl residue selected from the group consisting of —CH₂—O—CH₃, —CH₂—O—C₂H₅, —CH₂—O—CH(CH₃)₂, —CH₂—O—C(CH₃)₃, —CH₂—CH₂—O—CH₃, —CH₂—CH₂—O—C₂H₅, —CH₂—CH₂—O—CH(CH₃)₂, —CH₂—CH₂—O—C(CH₃)₃, —CH₂—O—CH₂—O—C₂H₅ and —CH₂—O—CH₂—O—CH(CH₃)₂; for a residue selected from the group consisting of piperidinyl, cyclopropyl, cyclobutyl, cyclopentyl, cyclohexyl, cycloheptyl, cyclopentenyl and cyclohexenyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NH—CO—(CH₂)₃—CH₃, —NO₂, —CF₃, —O—CF₃, —S—CF₃, —SH, —S—CH₃ and —S—C₂H₅; or represents a residue selected from the group consisting of phenyl, naphthyl, furanyl, thiophenyl, oxazolyl, oxadiazolyl, isoxazolyl, pyridinyl, pyridazinyl, pyrimidinyl, pyrazinyl, pyrazolyl, isoxazolyl, thiazolyl, quinolinyl, isoquinolinyl and quinazolinyl, which can respectively be bonded via a —(CH₂)—, —(CH₂)—(CH₂)—, —(CH₂)—(CH₂)—(CH₂)—, —(CH═CH)— or —(CH₂)—O— group and/or is unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
R¹⁶, R¹⁷, R¹⁸, R¹⁹, R²⁰, R²¹, R²², R²³, R²⁴, R²⁵ and R²⁶ each independently represent an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which is respectively unsubstituted or substituted with optionally 1, 2, 3, 4 or 5 substituents independently selected from the group consisting of F, Cl, Br, I, —NO₂, —CN, —OH, —SH, —NH₂, —C(═O)—OH, —C(═O)—O—CH₃, —C(═O)—O—C₂H₅ and —C(═O)—O—C(CH₃)₃; for a residue selected from the group consisting of cyclopropyl, cyclobutyl, cyclopentyl, cyclohexyl, cycloheptyl, cyclopentenyl and cyclohexenyl, which is respectively unsubstituted; or for a residue selected from the group consisting of furanyl, thienyl, quinolinyl, isoquinolinyl, phenyl and benzyl, which is respectively unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NO₂, —CF₃, —O—CF₃, —S—CF₃, —SH, —C(═O)—O—CH₃ and —C(═O)—O—C₂H₅;
   in each case optionally in the form of one of their pure stereoisomers, in particular enantiomers or diastereomers, their racemates, or in the form of a mixture of stereoisomers, in particular the enantiomers and/or diastereomers, in any mixture ratio, or respectively in the form of corresponding salts or respectively in the form of corresponding solvates.

In a further preferred embodiment the present invention relates to substituted 4-amino-quinazoline compounds corresponding to the foregoing formula I, wherein T, U, V and optionally W together with two carbon atoms form a ring selected from the group consisting of:

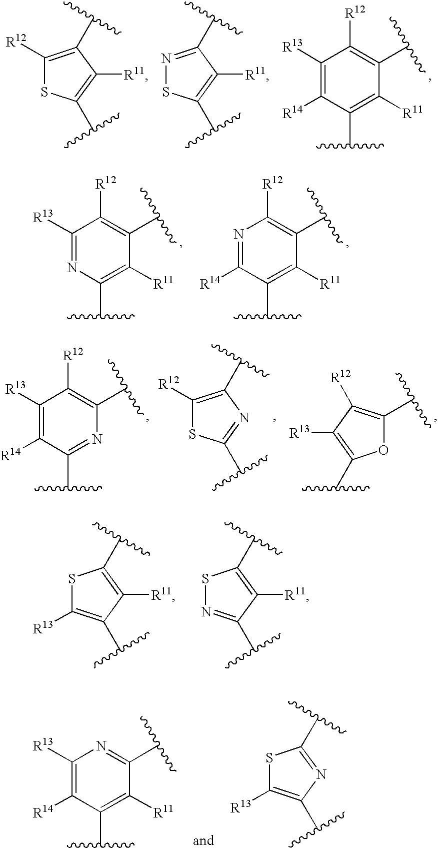

R¹ represents an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which is respectively unsubstituted or substituted with optionally 1, 2, 3, 4 or 5 substituents independently selected from the group consisting of F, Cl, Br, I, —NO₂, —CN, —OH, —OH₃, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; a residue selected from the group consisting of cyclopropyl, cyclobutyl, cyclopentyl, cyclohexyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or a residue selected from the group consisting of phenyl, benzyl and phenethyl, which is respectively unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅ and —CF₃;
R² represents H or an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; or
R¹ and R² together with the nitrogen atom to which they are bound form a residue selected from the group consisting of piperazinyl, piperidinyl, morpholinyl, thiomorpholinyl, pyrrolidinyl, azepanyl and diazepanyl, which is respectively unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —NO₂, —CF₃, —O—CF₃, —S—CF₃, —SH, —S—CH₃ and —S—C₂H₅;
R³ represents H; F; Cl; Br; I; —CN; —O—R²⁵; an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which is respectively unsubstituted or substituted with optionally 1, 2, 3, 4 or 5 substituents independently selected from the group consisting of F, Cl, Br, I, —CN and —OH;
R⁴, R⁵ and R⁶ each independently represent H; F; Cl; Br; I; —CN; —O—R²⁵; or an alkyl residue selected from the group consisting of methyl, ethyl, n-propyl, isopropyl, n-butyl, 2-butyl, isobutyl and tert-butyl, which is respectively unsubstituted or substituted with optionally 1, 2, 3, 4 or 5 substituents independently selected from the group consisting of F, Cl, Br, I, —NO₂ and —CN;
R⁷ and R⁸ each represent H;
R⁹ represents H; —C(═O)—CH₃; C(═O)—CH₂—CH₃; —C(═O)—O—R¹⁶; —S(═O)₂—R²¹; an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; for a —CH₂—CH═CH₂ or a —CH═CH—CH₃ residue, for a residue selected from the group consisting of cyclopropyl, cyclobutyl, cyclopentyl, cyclohexyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl and —CF₃; or for a residue selected from the group phenyl, pyridinyl, —(CH₂)-pyridinyl, thiazolyl, pyridazinyl, pyrimidinyl, pyrazinyl, furanyl, —(CH₂)-furanyl, —(CH₂)—(CH₂)-furanyl, thiophenyl, phenyl, naphthyl, benzyl and phenethyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH and —CF₃;
R¹⁰ represents —C(═O)—R¹⁵; —C(═O)—O—R¹⁶; —S(═O)—R²⁰; thiazolyl, or an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which is respectively substituted with 1, 2, 3, 4 or 5 substituents independently selected from the group consisting of F, Cl, Br, I, —NO₂, —CN, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃;
   and if R¹ represents a residue selected from the group consisting of methyl, ethyl, n-propyl, isopropyl, n-butyl, isobutyl, 2-butyl, tert-butyl and cyclopropyl, then R¹⁰ may additionally represent H or an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which can respectively be unsubstituted or substituted with 1, 2 or 3 substituents independently selected from the group consisting of —O—CH₃, —O—C₂H₅, —O—CH₂—C₂—CH₃, —O—CH(CH₃)₂ and —O—C(CH₃)₃; or
R⁹ and R¹⁰ together with the nitrogen atom linking them represent a residue selected from the group consisting of

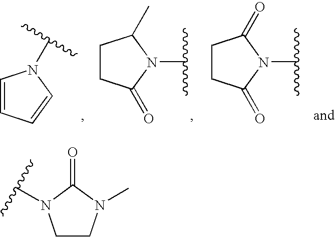

which is respectively unsubstituted or substituted with optionally 1, 2 or 3 substituents independently selected from the group consisting of methyl, ethyl, n-propyl, isopropyl, n-butyl, 2-butyl, isobutyl, tert-butyl, —NH—C(═O)—CF₃ and phenyl;
R¹¹, R¹², R¹³ and R¹⁴ each independently represent H; F; Cl; Br; I; —CN; —O—R²⁵; or an alkyl residue selected from the group consisting of methyl, ethyl, n-propyl, isopropyl, n-butyl, 2-butyl, isobutyl and tert-butyl, which is respectively unsubstituted or substituted with optionally 1, 2, 3, 4 or 5 substituents independently selected from the group consisting of F, Cl, Br, I, —NO₂, —CN, —OH, —SH and —NH₂;
R¹⁵ represents —C(═O)—O—R¹⁶; an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; for an alkenyl residue selected from the group consisting of ethenyl, 1-propenyl, 2-propenyl, 1-butenyl, 2-butenyl, 3-butenyl and —CH═C(CH₃)₂; for a heteroalkyl residue selected from the group consisting of —CH₂—O—CH₃, —CH₂—O—C₂H₅, —CH₂—O—CH(CH₃)₂, —CH₂—O—C(CH₃)₃, —CH₂—CH₂—O—CH₃, —CH₂—CH₂—O—C₂H₅, —CH₂—CH₂—O—CH(CH₃)₂, —CH₂—CH₂—O—C(CH₃)₃, —CH₂—O—CH₂—O—C₂H₅ and —CH₂—O—CH₂—O—CH(CH₃)₂; for a residue selected from the group consisting of piperidinyl, cyclopropyl, cyclobutyl, cyclopentyl, cyclohexyl, cycloheptyl, cyclopentenyl and cyclohexenyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NH—CO—(CH₂)₃—CH₃, —NO₂, —CF₃, —O—CF₃, —S—CF₃, —SH, —S—CH₃ and —S—C₂H₅; or a residue selected from the group consisting of phenyl, naphthyl, furanyl, thiophenyl, oxazolyl, oxadiazolyl, isoxazolyl, pyridinyl, pyridazinyl, pyrimidinyl, pyrazinyl, pyrazolyl, isoxazolyl, thiazolyl, quinolinyl, isoquinolinyl and quinazolinyl, which can be respectively bonded via a —(CH₂)—, —(CH₂)—(CH₂)—, —(CH₂)—(CH₂)—(CH₂)—, —(CH═CH)— or —(CH₂)—O— group and/or is unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
R¹⁶, R²⁰, R²¹ and R²⁵ each independently represent an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which is respectively unsubstituted or substituted with optionally 1, 2, 3, 4 or 5 substituents independently selected from the group consisting of F, Cl, Br, I, —NO₂, —CN, —OH, —SH, —NH₂, —C(═O)—OH, —C(═O)—O—CH₃, —C(═O)—O—C₂H₅ and —C(═O)—O—C(CH₃)₃; a residue selected from the group consisting of cyclopropyl, cyclobutyl, cyclopentyl, cyclohexyl, cycloheptyl, cyclopentenyl and cyclohexenyl, which is respectively unsubstituted; or a residue selected from the group consisting of furanyl, thienyl, quinolinyl, isoquinolinyl, phenyl and benzyl, which is respectively unsubstituted or is substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NO₂, —CF₃, —O—CF₃, —S—CF₃, —SH, —C(═O)—O—CH₃ and —C(═O)—O—C₂H₅;
   in each case optionally in the form of one of their pure stereoisomers, in particular enantiomers or diastereomers, their racemates, or in the form of a mixture of stereoisomers, in particular the enantiomers and/or diastereomers, in any mixture ratio, or respectively in the form of corresponding salts or respectively in the form of corresponding solvates.

Likewise highly preferred are substituted 4-amino-quinazoline compounds corresponding to formula Ia

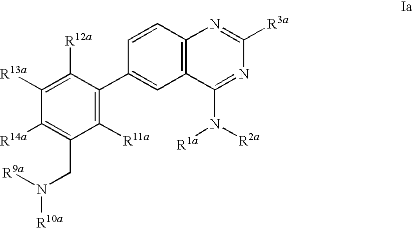

wherein
R^{1a} represents —C(═O)—R^{15a}; an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; a residue selected from the group consisting of cyclopropyl, cyclobutyl, cyclopentyl, cyclohexyl, cycloheptyl, cyclopentenyl and cyclohexenyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or a residue selected from the group consisting of phenyl, naphthyl, benzyl and phenethyl, which is respectively unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
R^{2a} represents H; —C(═O)—R^{15a}; or an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; or
R^{1a} and R^{2a} together with the nitrogen atom to which they are bound form a residue selected from the group consisting of piperazinyl, piperidinyl, morpholinyl, thiomorpholinyl, pyrrolidinyl, azepanyl and diazepanyl, which is respectively unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —NO₂, —CF₃, —O—CF₃, —S—CF₃, —SH, —S—CH₃ and —S—C₂H₅;
R^{3a} represents H; F; Cl; Br; I; —NO₂; —CN; —NH₂; —OH; —SH; —NH—R^{22a}; —NR^{23a}R^{24a}; —O—R^{25a}; —S—R^{26a}; an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃ and —C(═O)—O—C₂H₅; for a residue selected from the group consisting of morpholinyl, piperazinyl, piperidinyl, pyrrolidinyl, azepanyl, diazepanyl, thiomorpholinyl, cyclopropyl, cyclobutyl, cyclopentyl, cyclohexyl, cycloheptyl, cyclopentenyl and cyclohexenyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —NO₂, —CF₃, —O—CF₃, —S—CF₃, —SH, —S—CH₃ and —S—C₂H₅; or a residue selected from the group consisting of phenyl, benzyl, phenethyl, pyridinyl, pyridazinyl, pyrimidinyl and pyrazinyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
R^{9a} represents H; —C(═O)—R^{15a}; —C(═O)—O—R^{16a}; —S(═O)₂—R^{21a}; an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; for a heteroalkyl residue selected from the group consisting of —CH₂—O—CH₃, —CH₂—O—C₂H₅, —CH₂—O—CH(CH₃)₂, —CH₂—O—C(CH₃)₃, —CH₂—CH₂—O—CH₃, —CH₂—CH₂—O—C₂H₅, —CH₂—CH₂—O—CH(CH₃)₂, —CH₂—CH₂—O—C(CH₃)₃, —CH₂—O—CH₂—O—C₂H₅ and —CH₂—O—CH₂—O—CH(CH₃)₂; for a residue selected from the group consisting of cyclopropyl, cyclobutyl, cyclopentyl, cyclohexyl, cycloheptyl, cyclopentenyl and cyclohexenyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or represents a residue selected from the group consisting of pyridinyl, thiazolyl, pyridazinyl, pyrimidinyl, pyrazinyl, furanyl, —(CH₂)-furanyl, —(CH₂)—(CH₂)— furanyl, thiophenyl, phenyl, naphthyl, benzyl and phenethyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
R^{10a} represents —C(═O)—R^{15a}; —C(═O)—O—R^{16a}; —C(═O)—NH—R^{17a}; —C(═O)—NR^{18a}R^{19a}; —S(═O)—R^{20a}; —S(═O)₂—R^{21a}; or for an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which is respectively substituted with 1, 2, 3, 4 or 5 substituents independently selected from the group consisting of F, Cl, Br, I, —NO₂, —CN, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃;
   and if R^{1a} represents a residue selected from the group consisting of methyl, ethyl, n-propyl, isopropyl, n-butyl, isobutyl, 2-butyl, tert-butyl and cyclopropyl, then R^{10a} may additionally represent H or an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which can respectively be unsubstituted or substituted with 1, 2 or 3 substituents independently selected from the group consisting of —O—CH₃, —O—C₂H₅, —O—CH₂—C₂—CH₃, —O—CH(CH₃)₂ and —O—C(CH₃)₃; or
R^{9a} and R^{10a} together with the nitrogen atom to which they are bound form a residue selected from the group consisting of imidazolidinyl, azepanyl, pyrrolyl, diazepanyl, isothioazolidinyl, (2,3)-dihydro-1H-isoindolyl, indolinyl, octahydro-1H-isoindolyl, (2,3)-dihydropyrrolyl, (2,5)-dihydropyrrolyl, 8-azaspiro[4.5]decyl, 8-azaspiro[4.5]decane-7,9-dionyl, isoindoline-1,3-dionyl, pyrrolidine-2,5-dionyl, morpholine-3,5-dionyl, 1H-pyrrole-2,5-dionyl, piperidine-2,6-dionyl, hexahydro-2H-isoindoline-1,3-dionyl, pyrrolidine-2-onyl and 1,1-dioxo-1λ⁶-isothiazolidinyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, thioxo, —O—CH₃, —O—C₂H₅, —O—C₃H₇, —NH—C(═O)—CF₃ and phenyl; wherein the phenyl residue can be unsubstituted or substituted with 1, 2, 3, 4 or 5 substituents independently selected from the group consisting of F, Cl, Br, I, —CN, —CF₃, —O—CF₃, —O—CH₃, —O—C₂H₅, —O—C₃H₇, methyl, ethyl, n-propyl, isopropyl, n-butyl, 2-butyl, isobutyl and tert-butyl;
R^{11a}, R^{12a}, R^{13a} and R^{14a} each independently represent H; F; Cl; Br; I; —NO₂; —CN; —O—R²⁵; —S—R²⁶; or an alkyl residue selected from the group consisting of methyl, ethyl, n-propyl, isopropyl, n-butyl, 2-butyl, isobutyl and tert-butyl, which is respectively unsubstituted or substituted with optionally 1, 2, 3, 4 or 5 substituents independently selected from the group consisting of F, Cl, Br, I, —NO₂, —CN, —OH, —SH and —NH₂;
R^{15a} represents —C(═O)—O—R^{16a}; an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; an alkenyl residue selected from the group consisting of ethenyl, 1-propenyl, 2-propenyl, 1-butenyl, 2-butenyl, 3-butenyl and —CH═C(CH₃)₂; a heteroalkyl residue selected from the group consisting of —CH₂—O—CH₃, —CH₂—O—C₂H₅, —CH₂—O—CH(CH₃)₂, —CH₂—O—C(CH₃)₃, —CH₂—CH₂—O—CH₃, —CH₂—CH₂—O—C₂H₅, —CH₂—CH₂—O—CH(CH₃)₂, —CH₂—CH₂—O—C(CH₃)₃, —CH₂—O—CH₂—O—C₂H₅ and —CH₂—O—CH₂—O—CH(CH₃)₂; a residue selected from the group consisting of cyclopropyl, cyclobutyl, cyclopentyl, cyclohexyl, cycloheptyl, cyclopentenyl and cyclohexenyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or represents a residue selected from the group consisting of phenyl, naphthyl, furanyl, thiophenyl, oxazolyl, oxadiazolyl, isoxazolyl, pyridinyl, pyridazinyl, pyrimidinyl, pyrazinyl, quinolinyl, isoquinolinyl and quinazolinyl, which can respectively be bonded via a —(CH₂)—, —(CH₂)—(CH₂)—, —(CH₂)—(CH₂)—(CH₂)—, —(CH═CH)— or —(CH₂)—O— group and/or is unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
R^{16a}, R^{17a}, R^{18a}, R^{19a}, R^{20a}, R^{21a}, R^{22a}, R^{23a}, R^{24a}, R^{25a} and R^{26a} each independently represent an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which is respectively unsubstituted or substituted with optionally 1, 2, 3, 4 or 5 substituents independently selected from the group consisting of F, Cl, Br, I, —NO₂, —CN, —OH, —SH, —NH₂, —C(═O)—OH, —C(═O)—O—CH₃, —C(═O)—O—C₂H₅ and —C(═O)—O—C(CH₃)₃; a residue selected from the group consisting of cyclopropyl, cyclobutyl, cyclopentyl, cyclohexyl, cycloheptyl, cyclopentenyl and cyclohexenyl, which is respectively unsubstituted; or a residue selected from the group consisting of phenyl and benzyl, which is respectively unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NO₂, —CF₃, —O—CF₃, —S—CF₃, —SH, —C(═O)—O—CH₃ and —C(═O)—O—C₂H₅;
   in each case optionally in the form of one of their pure stereoisomers, in particular enantiomers or diastereomers, their racemates, or in the form of a mixture of stereoisomers, in particular the enantiomers and/or diastereomers, in any mixture ratio, or respectively in the form of corresponding salts or respectively in the form of corresponding solvates.

Likewise highly preferred are substituted 4-amino-quinazoline compounds corresponding to formula Ib

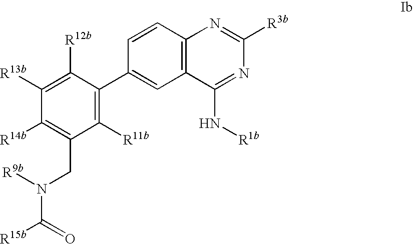

wherein
R^{1b} represents a residue selected from the group consisting of methyl, ethyl, n-propyl, isopropyl, n-butyl, 2-butyl, isobutyl, tert-butyl, n-pentyl, sec-pentyl, neo-pentyl, n-hexyl, —(CH₂)—C(═O)—OH and —(CH₂)—(CH₂)—C(═O)—OH; a residue selected from the group consisting of cyclopropyl, cyclobutyl, cyclopentyl, cyclohexyl, cycloheptyl, cyclopentenyl and cyclohexenyl; or a residue selected from the group consisting of phenyl, benzyl and phenethyl;
R^{3b} represents H; F; Cl; Br; —NH₂; —NH—R^{22b}; —NR^{23b}R^{24b}; an alkyl residue selected from the group consisting of methyl, ethyl, n-propyl, isopropyl, n-butyl, 2-butyl, isobutyl, tert-butyl, n-pentyl, sec-pentyl, neo-pentyl, n-hexyl, —CF₃, —CHF₂, —CH₂F and —CF₂—CF₃; a residue selected from the group consisting of morpholinyl, piperazinyl, piperidinyl, pyrrolidinyl, azepanyl, diazepanyl and thiomorpholinyl; or a residue selected from the group consisting of phenyl, benzyl, phenethyl and pyridinyl;
R^{9b} represents H; —C(═O)—O—R^{16b}; an alkyl residue selected from the group consisting of methyl, ethyl, n-propyl, isopropyl, n-butyl, 2-butyl, isobutyl, tert-butyl, n-pentyl, sec-pentyl, neo-pentyl, n-hexyl, —(CH₂)—(CH₂)—NH—C(═O)—O—C(CH₃)₃, —(CH₂)—NH—C(═O)—O—C(CH₃)₃, —(CH₂)—CN, —(CH₂)—(CH₂)—CN, —(CH₂)—Cl, —(CH₂)—(CH₂)—Cl, —(CH₂)—NH₂, —(CH₂)—(CH₂)—NH₂, —(CH₂)—C(═O)—O—CH₃, —(CH₂)—(CH₂)—C(═O)—O—CH₃ and —(CH₂)—C(═O)—O—C₂H₅; for a heteroalkyl residue selected from the group consisting of —CH₂—O—CH₃, —CH₂—O—C₂H₅, —CH₂—O—CH(CH₃)₂, —CH₂—O—C(CH₃)₃, —CH₂—CH₂—O—CH₃ and —CH₂—CH₂—O—C₂H₅; a residue selected from the group consisting of cyclopropyl, cyclobutyl, cyclopentyl, cyclohexyl, cycloheptyl, cyclopentenyl and cyclohexenyl; or a residue selected from the group consisting of pyridinyl, thiazolyl, —(CH₂)-furanyl, —(CH₂)—(CH₂)— furanyl, thiophenyl, phenyl, benzyl and phenethyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —O—CH₃, —O—C₂H₅ and —O—C₃H₇;
R^{11b} represents H; F; Cl; Br; —O—CH₃; —O—C₂H₅; —O—CH(CH₃)₂; —O—C(CH₃)₃; methyl; ethyl; n-propyl or isopropyl;
R^{12b} represents H; F; Cl; Br; —O—CH₃; —O—C₂H₅; —O—CH(CH₃)₂; —O—C(CH₃)₃; methyl; ethyl; n-propyl or isopropyl;
R^{13b} represents H; F; Cl; Br; —O—CH₃; —O—C₂H₅; —O—CH(CH₃)₂; —O—C(CH₃)₃; methyl; ethyl; n-propyl or isopropyl;
R^{14b} represents H; F; Cl; Br; —O—CH₃; —O—C₂H₅; —O—CH(CH₃)₂; —O—C(CH₃)₃; methyl; ethyl; n-propyl or isopropyl;
R^{15b} represents —C(═O)—O—R^{16b}; an alkyl residue selected from the group consisting of methyl, ethyl, n-propyl, isopropyl, n-butyl, 2-butyl, isobutyl, tert-butyl, n-pentyl, sec-pentyl, neo-pentyl, n-hexyl, —CF₃, —(CH₂)—F, —CHF₂, —(CH₂)—Cl, —(CH₂)—CF₃, —CF₂—CF₃, —(CH₂)—CN, —(CH₂)—N(CH₃)₂, —(CH₂)—(CH₂)—CN, —(CH₂)—(CH₂)—N(CH₃)₂, —(CH₂)—C(═O)—O—CH₃, —(CH₂)—C(═O)—OH, —(CH₂)—(CH₂)—C(═O)—O—CH₃ and —(CH₂)—(CH₂)—C(═O)—OH; an alkenyl residue selected from the group consisting of ethenyl, 1-propenyl, 2-propenyl, 1-butenyl, 2-butenyl, 3-butenyl and —CH═C(CH₃)₂; a heteroalkyl residue selected from the group consisting of —CH₂—O—CH₃, —CH₂—O—C₂H₅, —CH₂—O—CH(CH₃)₂, —CH₂—O—C(CH₃)₃, —CH₂—CH₂—O—CH₃ and —CH₂—CH₂—O—C₂H₅; a residue selected from the group consisting of cyclopropyl, cyclobutyl, cyclopentyl, cyclohexyl, cycloheptyl, cyclopentenyl and cyclohexenyl; a residue selected from the group consisting of phenyl and pyridinyl, which is respectively bonded via a —(CH₂)—, —(CH₂)—(CH₂)—, —(CH₂)—(CH₂)—(CH₂)—, —(CH═CH)— or —(CH₂)—O— group and/or is unsubstituted or substituted with optionally 1, 2, 3, 4 or 5 substituents independently selected from the group consisting of F, Cl, Br, I, —C(═O)—OH, —C(═O)—O—CH₃ and —C(═O)—O—C₂H₅; or a residue selected from the group consisting of phenyl, furanyl, thiophenyl, oxazolyl, isoxazolyl, pyridinyl, pyrazinyl, quinolinyl and isoquinolinyl, which is respectively substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NO₂, —CF₃, —O—CF₃ and —S—CF₃;
R^{16b} represents an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl; and
R^{22b}, R^{23b} and R^{24b} each independently represent an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl; a residue selected from the group consisting of cyclopropyl, cyclobutyl, cyclopentyl, cyclohexyl, cycloheptyl, cyclopentenyl and cyclohexenyl; or a residue selected from the group consisting of phenyl and benzyl;
   in each case optionally in the form of one of their pure stereoisomers, in particular enantiomers or diastereomers, their racemates, or in the form of a mixture of stereoisomers, in particular the enantiomers and/or diastereomers, in any mixture ratio, or respectively in the form of corresponding salts or respectively in the form of corresponding solvates.

Also highly preferred are substituted 4-amino-quinazoline compounds corresponding to the foregoing formula Ib in which:
R^{1b} represents a residue selected from the group consisting of methyl, ethyl, n-propyl, isopropyl, n-butyl, 2-butyl, isobutyl, tert-butyl, n-pentyl, sec-pentyl, neo-pentyl, n-hexyl, —(CH₂)—C(═O)—OH and —(CH₂)—(CH₂)—C(═O)—OH;
R^{3b} represents H;
R^{9b} represents H; —C(═O)—OR^{16b}; an alkyl residue selected from the group consisting of methyl, ethyl, n-propyl, isopropyl, n-butyl, 2-butyl, isobutyl, tert-butyl, n-pentyl, sec-pentyl, neo-pentyl, n-hexyl, —(CH₂)—(CH₂)—NH—C(═O)—O—C(CH₃)₃, —(CH₂)—NH—C(═O)—O—C(CH₃)₃, —(CH₂)—CN, —(CH₂)—(CH₂)—CN, —(CH₂)—Cl, —(CH₂)—(CH₂)—Cl, —(CH₂)—NH₂, —(CH₂)—(CH₂)—NH₂, —(CH₂)—C(═O)—O—CH₃, —(CH₂)—(CH₂)—C(═O)—O—CH₃ and —(CH₂)—C(═O)—O—C₂H₅; for a heteroalkyl residue selected from the group consisting of —CH₂—O—CH₃, —CH₂—O—C₂H₅, —CH₂—O—CH(CH₃)₂, —CH₂—O—C(CH₃)₃, —CH₂—CH₂—O—CH₃ and —CH₂—CH₂—O—C₂H₅; a residue selected from the group consisting of cyclopropyl, cyclobutyl, cyclopentyl, cyclohexyl, cycloheptyl, cyclopentenyl and cyclohexenyl; or a residue selected from the group consisting of pyridinyl, thiazolyl, —(CH₂)-furanyl, —(CH₂)—(CH₂)— furanyl, thiophenyl, phenyl, benzyl and phenethyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —O—CH₃, —O—C₂H₅ and —O—C₃H₇;
R^{11b} represents H;
R^{12b} represents H;
R^{13b} represents H;
R^{14b} represents H;
R^{15b} represents —C(═O)—O—R^{16b}; an alkyl residue selected from the group consisting of methyl, ethyl, n-propyl, isopropyl, n-butyl, 2-butyl, isobutyl, tert-butyl, n-pentyl, sec-pentyl, neo-pentyl, n-hexyl, —CF₃, —(CH₂)—F, —CHF₂, —(CH₂)—Cl, —(CH₂)—CF₃, —CF₂—CF₃, —(CH₂)—CN, —(CH₂)—N(CH₃)₂, —(CH₂)—(CH₂)—CN, —(CH₂)—(CH₂)—N(CH₃)₂, —(CH₂)—C(═O)—O—CH₃, —(CH₂)—C(═O)—OH, —(CH₂)—(CH₂)—C(═O)—O—CH₃ and —(CH₂)—(CH₂)—C(═O)—OH; an alkenyl residue selected from the group consisting of ethenyl, 1-propenyl, 2-propenyl, 1-butenyl, 2-butenyl, 3-butenyl and —CH═C(CH₃)₂; a heteroalkyl residue selected from the group consisting of —CH₂—O—CH₃, —CH₂—O—C₂H₅, —CH₂—O—CH(CH₃)₂, —CH₂—O—C(CH₃)₃, —CH₂—CH₂—O—CH₃ and —CH₂—CH₂—O—C₂H₅; a residue selected from the group consisting of cyclopropyl, cyclobutyl, cyclopentyl, cyclohexyl, cycloheptyl, cyclopentenyl and cyclohexenyl; a residue selected from the group consisting of phenyl and pyridinyl, which is respectively bonded via a —(CH₂)—, —(CH₂)—(CH₂)—, —(CH₂)—(CH₂)—(CH₂)—, —(CH═CH)— or —(CH₂)—O— group and/or is unsubstituted or substituted with optionally 1, 2, 3, 4 or 5 substituents independently selected from the group consisting of F, Cl, Br, I, —C(═O)—OH, —C(═O)—O—CH₃ and —C(═O)—O—C₂H₅; or a residue selected from the group consisting of phenyl, furanyl, thiophenyl, oxazolyl, isoxazolyl, pyridinyl, pyrazinyl, quinolinyl and isoquinolinyl, which is respectively substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NO₂, —CF₃, —O—CF₃ and —S—CF₃; and
R^{16b} represents an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl;
   in each case optionally in the form of one of their pure stereoisomers, in particular enantiomers or diastereomers, their racemates, or in the form of a mixture of stereoisomers, in particular the enantiomers and/or diastereomers, in any mixture ratio, or respectively in the form of corresponding salts or respectively in the form of corresponding solvates.

Also highly preferred are substituted 4-amino-quinazoline compounds corresponding to formula Ic

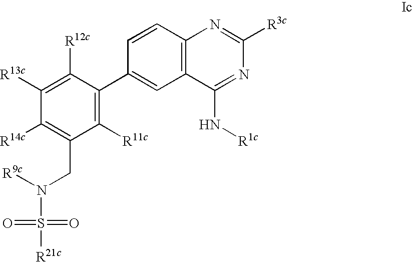

wherein
R^{1c} represents a residue selected from the group consisting of methyl, ethyl, n-propyl, isopropyl, n-butyl, 2-butyl, isobutyl, tert-butyl, n-pentyl, sec-pentyl, neo-pentyl, n-hexyl, —(CH₂)—C(═O)—OH and —(CH₂)—(CH₂)—C(═O)—OH; a residue selected from the group consisting of cyclopropyl, cyclobutyl, cyclopentyl, cyclohexyl, cycloheptyl, cyclopentenyl and cyclohexenyl; or a residue selected from the group consisting of phenyl, benzyl and phenethyl;
R^{3c} represents H; F; Cl; Br; —NH₂; —NH—R^{22c}; —NR^{23c}R^{24c}; an alkyl residue selected from the group consisting of methyl, ethyl, n-propyl, isopropyl, n-butyl, 2-butyl, isobutyl, tert-butyl, n-pentyl, sec-pentyl, neo-pentyl, n-hexyl, —CF₃, —CHF₂, —CH₂F and —CF₂—CF₃; a residue selected from the group consisting of morpholinyl, piperazinyl, piperidinyl, pyrrolidinyl, azepanyl, diazepanyl and thiomorpholinyl; or a residue selected from the group consisting of phenyl, benzyl, phenethyl and pyridinyl;
R^{9c} represents H; —C(═O)—O—R^{16c}; an alkyl residue selected from the group consisting of methyl, ethyl, n-propyl, isopropyl, n-butyl, 2-butyl, isobutyl, tert-butyl, n-pentyl, sec-pentyl, neo-pentyl, n-hexyl, —(CH₂)—(CH₂)—NH—C(═O)—O—C(CH₃)₃, —(CH₂)—NH—C(═O)—O—C(CH₃)₃, —(CH₂)—CN, —(CH₂)—(CH₂)—CN, —(CH₂)—Cl, —(CH₂)—(CH₂)—Cl, —(CH₂)—NH₂, —(CH₂)—(CH₂)—NH₂, —(CH₂)—C(═O)—O—CH₃, —(CH₂)—(CH₂)—C(═O)—O—CH₃ and —(CH₂)—C(═O)—O—C₂H₅; a heteroalkyl residue selected from the group consisting of —CH₂—O—CH₃, —CH₂—O—C₂H₅, —CH₂—O—CH(CH₃)₂, —CH₂—O—C(CH₃)₃, —CH₂—CH₂—O—CH₃ and —CH₂—CH₂—O—C₂H₅; a residue selected from the group consisting of cyclopropyl, cyclobutyl, cyclopentyl, cyclohexyl, cycloheptyl, cyclopentenyl and cyclohexenyl; or a residue selected from the group consisting of pyridinyl, thiazolyl, —(CH₂)-furanyl, —(CH₂)—(CH₂)— furanyl, thiophenyl, phenyl, benzyl and phenethyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —O—CH₃, —O—C₂H₅ and —O—C₃H₇;
R^{11c} represents H; F; Cl; Br; —O—CH₃; —O—C₂H₅; —O—CH(CH₃)₂; —O—C(CH₃)₃; methyl; ethyl; n-propyl or isopropyl;
R^{12c} represents H; F; Cl; Br; —O—CH₃; —O—C₂H₅; —O—CH(CH₃)₂; —O—C(CH₃)₃; methyl; ethyl; n-propyl or isopropyl;
R^{13c} represents H; F; Cl; Br; —O—CH₃; —O—C₂H₅; —O—CH(CH₃)₂; —O—C(CH₃)₃; methyl; ethyl; n-propyl or isopropyl;
R^{14c} represents H; F; Cl; Br; —O—CH₃; —O—C₂H₅; —O—CH(CH₃)₂; —O—C(CH₃)₃; methyl; ethyl; n-propyl or isopropyl;
R^{16c} represents an alkyl residue selected from the group consisting of methyl, ethyl, n-propyl, isopropyl, n-butyl, 2-butyl, isobutyl, tert-butyl, n-pentyl, sec-pentyl, neo-pentyl, and n-hexyl;
R^{21c} represents an alkyl residue selected from the group consisting of methyl, ethyl, n-propyl, isopropyl, n-butyl, 2-butyl, isobutyl, tert-butyl, n-pentyl, sec-pentyl, neo-pentyl, n-hexyl, —CF₃, —(CH₂)—F, —CHF₂, —(CH₂)—Cl, —(CH₂)—CF₃, —CF₂—CF₃, —(CH₂)—CN, —(CH₂)—N(CH₃)₂, —(CH₂)—(CH₂)—CN, —(CH₂)—(CH₂)—N(CH₃)₂, —(CH₂)—C(═O)—O—CH₃, —(CH₂)—C(═O)—OH, —(CH₂)—(CH₂)—C(═O)—O—CH₃ and —(CH₂)—(CH₂)—C(═O)—OH; a residue selected from the group consisting of cyclopropyl, cyclobutyl, cyclopentyl, cyclohexyl, cycloheptyl, cyclopentenyl and cyclohexenyl, which is respectively unsubstituted; or a residue selected from the group consisting of phenyl and benzyl, which is respectively unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NO₂, —CF₃, —O—CF₃, —S—CF₃, —SH, —C(═O)—O—CH₃ and —C(═O)—O—C₂H₅; and
R^{22c}, R^{23c} and R^{24c} each independently represent an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl; a residue selected from the group consisting of cyclopropyl, cyclobutyl, cyclopentyl, cyclohexyl, cycloheptyl, cyclopentenyl and cyclohexenyl; or a residue selected from the group consisting of phenyl and benzyl;
   in each case optionally in the form of one of their pure stereoisomers, in particular enantiomers or diastereomers, their racemates, or in the form of a mixture of stereoisomers, in particular the enantiomers and/or diastereomers, in any mixture ratio, or respectively in the form of corresponding salts or respectively in the form of corresponding solvates.

Also highly preferred are substituted 4-amino-quinazoline compounds corresponding to the foregoing formula Ic in which:
R^{1c} represents a residue selected from the group consisting of methyl, ethyl, n-propyl, isopropyl, n-butyl, 2-butyl, isobutyl, tert-butyl, n-pentyl, sec-pentyl, neo-pentyl, n-hexyl, —(CH₂)—C(═O)—OH and —(CH₂)—(CH₂)—C(═O)—OH;
R^{3c} represents H;
R^{9c} represents H; —C(═O)—O—R^{16c}; an alkyl residue selected from the group consisting of methyl, ethyl, n-propyl, isopropyl, n-butyl, 2-butyl, isobutyl, tert-butyl, n-pentyl, sec-pentyl, neo-pentyl, n-hexyl, —(CH₂)—(CH₂)—NH—C(═O)—O—C(CH₃)₃, —(CH₂)—NH—C(═O)—O—C(CH₃)₃, —(CH₂)—CN, —(CH₂)—(CH₂)—CN, —(CH₂)—Cl, —(CH₂)—(CH₂)—Cl, —(CH₂)—NH₂, —(CH₂)—(CH₂)—NH₂, —(CH₂)—C(═O)—O—CH₃, —(CH₂)—(CH₂)—C(═O)—O—CH₃ and —(CH₂)—C(═O)—O—C₂H₅; for a heteroalkyl residue selected from the group consisting of —CH₂—O—CH₃, —CH₂—O—C₂H₅, —CH₂—O—CH(CH₃)₂, —CH₂—O—C(CH₃)₃, —CH₂—CH₂—O—CH₃ and —CH₂—CH₂—O—C₂H₅; for a residue selected from the group consisting of cyclopropyl, cyclobutyl, cyclopentyl, cyclohexyl, cycloheptyl, cyclopentenyl and cyclohexenyl; or for a residue selected from the group consisting of pyridinyl, thiazolyl, —(CH₂)-furanyl, —(CH₂)—(CH₂)-furanyl, thiophenyl, phenyl, benzyl and phenethyl, which is respectively unsubstituted or is substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —O—CH₃, —O—C₂H₅ and —O—C₃H₇;
R^{11c} represents H;
R^{12c} represents H;
R^{13c} represents H;
R^{14c} represents H;
R^{16c} represents an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl; and
R^{21c} represents an alkyl residue selected from the group consisting of methyl, ethyl, n-propyl, isopropyl, n-butyl, 2-butyl, isobutyl, tert-butyl, n-pentyl, sec-pentyl, neo-pentyl, n-hexyl, —CF₃, —CHF₂, —CH₂F, —CF₂—CF₃, —(CH₂)—CF₃, —(CH₂)—Cl, —(CH₂)—(CH₂)—Cl, —(CH₂)—(CH₂)—(CH₂)—Cl and —(CH₂)—(CH₂)—(CH₂)—(CH₂)—Cl; or a residue selected from the group consisting of phenyl and benzyl, which is respectively unsubstituted or substituted with 1, 2, 3, 4 or 5 substituents independently selected from the group consisting of F, Cl, Br and —CF₃;
   in each case optionally in the form of one of their pure stereoisomers, in particular enantiomers or diastereomers, their racemates, or in the form of a mixture of stereoisomers, in particular the enantiomers and/or diastereomers, in any mixture ratio, or respectively in the form of corresponding salts or respectively in the form of corresponding solvates.

Even more preferred are substituted 4-amino-quinazoline compounds selected from the group consisting of:
1 6-hydroxy-N-isobutyl-N-[3-(4-morpholin-4-yl-quinazolin-6-yl)-benzyl]-nicotinamide,
2 N-cyclopropyl-6-hydroxy-N-[3-(4-pyrrolidin-1-yl-quinazolin-6-yl)-benzyl]-nicotinamide,
3 N-cyclopropyl-N-[3-(4-cyclopropylamino-quinazolin-6-yl)-benzyl]-2,4-difluorobenzamide,
4 thiophene-3-carboxylic acid cyclopropyl-[3-(4-cyclopropylamino-quinazolin-6-yl)-benzyl]-amide,
5 N-isobutyl-N-[3-(4-morpholin-4-yl-quinazolin-6-yl)-benzyl]-benzamide,
6 2-bromo-N-isobutyl-N-[3-(4-morpholin-4-yl-quinazolin-6-yl)-benzyl]-benzamide,
7 N-isobutyl-3,4-dimethoxy-N-[3-(4-morpholin-4-yl-quinazolin-6-yl)-benzyl]-benzamide,
8 2,4-difluoro-N-isobutyl-N-[3-(4-morpholin-4-yl-quinazolin-6-yl)-benzyl]-benzamide,
9 isoquinoline-3-carboxylic acid isobutyl-[3-(4-morpholin-4-yl-quinazolin-6-yl)-benzyl]-amide,
10 N-isobutyl-N-[3-(4-morpholin-4-yl-quinazolin-6-yl)-benzyl]-3-phenyl-acrylamide,
11 4-chloro-N-isobutyl-N-[3-(4-morpholin-4-yl-quinazolin-6-yl)-benzyl]-benzamide,
12 4-cyano-N-isobutyl-N-[3-(4-morpholin-4-yl-quinazolin-6-yl)-benzyl]-benzamide,
13 pyrazine-2-carboxylic acid isobutyl-[3-(4-morpholin-4-yl-quinazolin-6-yl)-benzyl]-amide,
14 6-bromo-pyridine-2-carboxylic acid isobutyl-[3-(4-morpholin-4-yl-quinazolin-6-yl)-benzyl]-amide,
15 N-isobutyl-N-[3-(4-morpholin-4-yl-quinazolin-6-yl)-benzyl]-2-phenoxy-acetamide,
16 N-isobutyl-N-[3-(4-morpholin-4-yl-quinazolin-6-yl)-benzyl]-2-pyridin-2-yl-acetamide,
17 3-cyano-N-isobutyl-N-[3-(4-morpholin-4-yl-quinazolin-6-yl)-benzyl]-benzamide,
18 thiophene-3-carboxylic acid isobutyl-[3-(4-morpholin-4-yl-1 quinazolin-6-yl)-benzyl]-amide,
19 6-chloro-N-isobutyl-N-[3-(4-morpholin-4-yl-quinazolin-6-yl)-benzyl]-nicotinamide,
20 3-methyl-thiophene-2-carboxylic acid isobutyl-[3-(4-morpholin-4-yl-quinazolin-6-yl)-benzyl]-amide,
21 2-cyano-N-isobutyl-N-[3-(4-morpholin-4-yl-quinazolin-6-yl)-benzyl]-acetamide,
22 N-isobutyl-4-methyl-N-[3-(4-morpholin-4-yl-quinazolin-6-yl)-benzyl]-benzamide,
23 N-isobutyl-N-[3-(4-morpholin-4-yl-quinazolin-6-yl)-benzyl]-2-trifluoromethyl-benzamide,
24 5-bromo-thiophene-2-carboxylic acid isobutyl-[3-(4-morpholin-4-yl-quinazolin-6-yl)-benzyl]-amide,
25 N-isobutyl-N-[3-(4-morpholin-4-yl-quinazolin-6-yl)-benzyl]-isonicotinamide,
26 N-isobutyl-N-[3-(4-morpholin-4-yl-quinazolin-6-yl)-benzyl]-butyramide,
27 2-hydroxy-N-isobutyl-N-[3-(4-morpholin-4-yl-quinazolin-6-yl)-benzyl]-benzamide,
28 2-fluoro-N-isobutyl-N-[3-(4-morpholin-4-yl-quinazolin-6-yl)-benzyl]-4-trifluoromethyl-benzamide,
29 thiophene-2-carboxylic acid isobutyl-[3-(4-morpholin-4-yl-quinazolin-6-yl)-benzyl]-amide,
30 N-isobutyl-N-[3-(4-morpholin-4-yl-quinazolin-6-yl)-benzyl]-nicotinamide,
31 2-dimethylamino-N-isobutyl-N-[3-(4-morpholin-4-yl-quinazolin-6-yl)-benzyl]-acetamide,
32 N-isobutyl-4-methoxy-N-[3-(4-morpholin-4-yl-quinazolin-6-yl)-benzyl]-benzamide,
33 2-chloro-N-isobutyl-N-[3-(4-morpholin-4-yl-quinazolin-6-yl)-benzyl]-benzamide,
34 furan-2-carboxylic acid isobutyl-[3-(4-morpholin-4-yl-quinazolin-6-yl)-benzyl]-amide,
35 pyridine-2-carboxylic acid isobutyl-[3-(4-morpholin-4-yl-quinazolin-6-yl)-benzyl]-amide,
36 N-benzyl-N-[3-(4-isopropylamino-quinazolin-6-yl)-benzyl]-benzamide,
37 N-benzyl-2-bromo-N-[3-(4-isopropylamino-quinazolin-6-yl)-benzyl]-benzamide,
38 3-methyl-but-2-ene acid benzyl-[3-(4-isopropylamino-quinazolin-6-yl)-benzyl]-amide,
39 N-benzyl-N-[3-(4-isopropylamino-quinazolin-6-yl)-benzyl]-3,4-dimethoxy-benzamide,
40 N-benzyl-2,4-difluoro-N-[3-(4-isopropylamino-quinazolin-6-yl)-benzyl]-benzamide,
41 isoquinoline-3-carboxylic acid benzyl-[3-(4-isopropylamino-quinazolin-6-yl)-benzyl]-amide,
42 N-benzyl-N-[3-(4-isopropylamino-quinazolin-6-yl)-benzyl]-3-phenyl-acrylamide,
43 N-Benzyl-4-chloro-N-[3-(4-isopropylamino-quinazolin-6-yl)-benzyl]-benzamide,
44 N-benzyl-4-cyano-N-[3-(4-isopropylamino-quinazolin-6-yl)-benzyl]-benzamide,
45 pyrazine-2-carboxylic acid benzyl-[3-(4-isopropylamino-quinazolin-6-yl)-benzyl]-amide,
46 6-bromo-pyridine-2-carboxylic acid benzyl-[3-(4-isopropylamino-quinazolin-6-yl)-benzyl]-amide,
47 N-benzyl-N-[3-(4-isopropylamino-quinazolin-6-yl)-benzyl]-2-phenoxy-acetamide,
48 N-benzyl-N-[3-(4-isopropylamino-quinazolin-6-yl)-benzyl]-2-pyridin-2-yl-acetamide,
49 N-benzyl-3-cyano-N-[3-(4-isopropylamino-quinazolin-6-yl)-benzyl]-benzamide,
50 thiophene-3-carboxylic acid benzyl-[3-(4-isopropylamino-quinazolin-6-yl)-benzyl]-amide,
51 N-benzyl-6-chloro-N-[3-(4-isopropylamino-quinazolin-6-yl)-benzyl]-nicotinamide,
52 3-methyl-thiophene-2-carboxylic acid benzyl-[3-(4-isopropylamino-quinazolin-6-yl)-benzyl]-amide,
53 N-benzyl-2-cyano-N-[3-(4-isopropylamino-quinazolin-6-yl)-benzyl]-acetamide,
54 N-benzyl-N-[3-(4-isopropylamino-quinazolin-6-yl)-benzyl]-4-methyl-benzamide,
55 N-benzyl-N-[3-(4-isopropylamino-quinazolin-6-yl)-benzyl]-2-trifluoromethyl-benzamide,
56 N-benzyl-2-hydroxy-N-[3-(4-isopropylamino-quinazolin-6-yl)-benzyl]-benzamide,
57 N-[3-(4-diethylamino-quinazolin-6-yl)-benzyl]-N-methyl-benzamide,
58 2-bromo-N-[3-(4-diethylamino-quinazolin-6-yl)-benzyl]-N-methyl-benzamide,
59 N-[3-(4-diethylamino-quinazolin-6-yl)-benzyl]-3,4-dimethoxy-N-methyl-benzamide,
60 N-[3-(4-diethylamino-quinazolin-6-yl)-benzyl]-2,4-difluoro-N-methyl-benzamide,
61 isoquinoline-3-carboxylic acid [3-(4-diethylamino-quinazolin-6-yl)-benzyl]-methyl-amide,
62 N-[3-(4-diethylamino-quinazolin-6-yl)-benzyl]-N-methyl-3-phenyl-acrylamide,
63 4-chloro-N-[3-(4-diethylamino-quinazolin-6-yl)-benzyl]-N-methyl-benzamide,
64 4-cyano-N-[3-(4-diethylamino-quinazolin-6-yl)-benzyl]-N-methyl-benzamide,
65 pyrazine-2-carboxylic acid [3-(4-diethylamino-quinazolin-6-yl)-benzyl]-methyl-amide,
66 6-bromo-pyridine-2-carboxylic acid [3-(4-diethylamino-quinazolin-6-yl)-benzyl]-methyl-amide,
67 N-[3-(4-diethylamino-quinazolin-6-yl)-benzyl]-N-methyl-2-phenoxy-acetamide,
68 N-[3-(4-diethylamino-quinazolin-6-yl)-benzyl]-N-methyl-2-pyridin-2-yl-acetamide,
69 3-cyano-N-[3-(4-diethylamino-quinazolin-6-yl)-benzyl]-N-methyl-benzamide,
70 thiophene-3-carboxylic acid [3-(4-diethylamino-quinazolin-6-yl)-benzyl]-methyl-amide,
71 6-chloro-N-[3-(4-diethylamino-quinazolin-6-yl)-benzyl]-N-methyl-nicotinamide,
72 3-methyl-thiophene-2-carboxylic acid [3-(4-diethylamino-quinazolin-6-yl)-benzyl]-methyl-amide,
73 N-[3-(4-diethylamino-quinazolin-6-yl)-benzyl]-6-hydroxy-N-methyl-nicotinamide,
74 2-cyano-N-[3-(4-diethylamino-quinazolin-6-yl)-benzyl]-N-methyl-acetamide,
75 N-[3-(4-diethylamino-quinazolin-6-yl)-benzyl]-4,N-dimethyl-benzamide,
76 N-[3-(4-diethylamino-quinazolin-6-yl)-benzyl]-N-methyl-2-trifluoromethyl-benzamide,
77 5-bromo-thiophene-2-carboxylic acid [3-(4-diethylamino-quinazolin-6-yl)-benzyl]-methyl-amide,
78 N-[3-(4-diethylamino-quinazolin-6-yl)-benzyl]-N-methyl-isonicotinamide,
79 N-[3-(4-diethylamino-quinazolin-6-yl)-benzyl]-N-methyl-butyramide,
80 N-[3-(4-diethylamino-quinazolin-6-yl)-benzyl]-2-hydroxy-N-methyl-benzamide,
81 N-[3-(4-diethylamino-quinazolin-6-yl)-benzyl]-2-fluoro-N-methyl-4-trifluoromethyl-benzamide,
82 thiophene-2-carboxylic acid [3-(4-diethylamino-quinazolin-6-yl)-benzyl]-methyl-amide,
83 N-[3-(4-diethylamino-quinazolin-6-yl)-benzyl]-N-methyl-nicotinamide,
84 N-[3-(4-diethylamino-quinazolin-6-yl)-benzyl]-2-dimethylamino-N-methyl-acetamide,
85 N-[3-(4-diethylamino-quinazolin-6-yl)-benzyl]-4-methoxy-N-methyl-benzamide,
86 2-chloro-N-[3-(4-diethylamino-quinazolin-6-yl)-benzyl]-N-methyl-benzamide,
87 furan-2-carboxylic acid [3-(4-diethylamino-quinazolin-6-yl)-benzyl]-methyl-amide,
88 pyridine-2-carboxylic acid [3-(4-diethylamino-quinazolin-6-yl)-benzyl]-methyl-amide,
89 N-[3-(4-phenylamino-quinazolin-6-yl)-benzyl]-N-propyl-benzamide,
90 2-phenoxy-N-[3-(4-phenylamino-quinazolin-6-yl)-benzyl]-N-propyl-acetamide,
91 4-methyl-N-[3-(4-phenylamino-quinazolin-6-yl)-benzyl]-N-propyl-benzamide,
92 N-[3-(4-phenylamino-quinazolin-6-yl)-benzyl]-N-propyl-isonicotinamide,
93 2-fluoro-N-[3-(4-phenylamino-quinazolin-6-yl)-benzyl]-N-propyl-4-trifluoromethyl-benzamide,
94 4-methoxy-N-[3-(4-phenylamino-quinazolin-6-yl)-benzyl]-N-propyl-benzamide,
95 pyridine-2-carboxylic acid [3-(4-phenylamino-quinazolin-6-yl)-benzyl]-propyl-amide,
96 3-methyl-thiophene-2-carboxylic acid cyclopropyl-[3-(4-pyrrolidin-1-yl-quinazolin-6-yl)-benzyl]-amide,
97 2-bromo-N-[3-(4-phenylamino-quinazolin-6-yl)-benzyl]-N-propyl-benzamide,
98 3,4-dimethoxy-N-[3-(4-phenylamino-quinazolin-6-yl)-benzyl]-N-propyl-benzamide,
99 2,4-difluoro-N-[3-(4-phenylamino-quinazolin-6-yl)-benzyl]-N-propyl-benzamide,
100 isoquinoline-3-carboxylic acid [3-(4-phenylamino-quinazolin-6-yl)-benzyl]-propyl-amide,
101 3-phenyl-N-[3-(4-phenylamino-quinazolin-6-yl)-benzyl]-N-propyl-acrylamide,
102 4-chloro-N-[3-(4-phenylamino-quinazolin-6-yl)-benzyl]-N-propyl-benzamide,
103 4-cyano-N-[3-(4-phenylamino-quinazolin-6-yl)-benzyl]-N-propyl-benzamide,
104 pyrazine-2-carboxylic acid [3-(4-phenylamino-quinazolin-6-yl)-benzyl]-propyl-amide,
105 6-bromo-pyridine-2-carboxylic acid [3-(4-phenylamino-quinazolin-6-yl)-benzyl]-propyl-amide,
106 N-[3-(4-phenylamino-quinazolin-6-yl)-benzyl]-N-propyl-2-pyridin-2-yl-acetamide,
107 3-cyano-N-[3-(4-phenylamino-quinazolin-6-yl)-benzyl]-N-propyl-benzamide,
108 thiophene-3-carboxylic acid [3-(4-phenylamino-quinazolin-6-yl)-benzyl]-propyl-amide,
109 6-chloro-N-[3-(4-phenylamino-quinazolin-6-yl)-benzyl]-N-propyl-nicotinamide,
110 3-methyl-thiophene-2-carboxylic acid [3-(4-phenylamino-quinazolin-6-yl)-benzyl]-propyl-amide,
111 6-hydroxy-N-[3-(4-phenylamino-quinazolin-6-yl)-benzyl]-N-propyl-nicotinamide,
112 2-cyano-N-[3-(4-phenylamino-quinazolin-6-yl)-benzyl]-N-propyl-acetamide,
113 N-[3-(4-phenylamino-quinazolin-6-yl)-benzyl]-N-propyl-2-trifluoromethyl-benzamide,
114 5-bromo-thiophene-2-carboxylic acid [3-(4-phenylamino-quinazolin-6-yl)-benzyl]-propyl-amide,
115 N-[3-(4-phenylamino-quinazolin-6-yl)-benzyl]-N-propyl-butyramide,
116 thiophene-2-carboxylic acid [3-(4-phenylamino-quinazolin-6-yl)-benzyl]-propyl-amide,
117 N-[3-(4-phenylamino-quinazolin-6-yl)-benzyl]-N-propyl-nicotinamide,
118 2-dimethylamino-N-[3-(4-phenylamino-quinazolin-6-yl)-benzyl]-N-propyl-acetamide,
119 2-chloro-N-[3-(4-phenylamino-quinazolin-6-yl)-benzyl]-N-propyl-benzamide,
120 furan-2-carboxylic acid [3-(4-phenylamino-quinazolin-6-yl)-benzyl]-propyl-amide,
121 N-cyclopropyl-N-[3-(4-pyrrolidin-1-yl-quinazolin-6-yl)-benzyl]-benzamide,
122 2-bromo-N-cyclopropyl-N-[3-(4-pyrrolidin-1-yl-quinazolin-6-yl)-benzyl]-benzamide,
123 N-cyclopropyl-3,4-dimethoxy-N-[3-(4-pyrrolidin-1-yl-quinazolin-6-yl)-benzyl]-benzamide,
124 N-cyclopropyl-2,4-difluoro-N-[3-(4-pyrrolidin-1-yl-quinazolin-6-yl)-benzyl]-benzamide,
125 isoquinoline-3-carboxylic acid cyclopropyl-[3-(4-pyrrolidin-1-yl-quinazolin-6-yl)-benzyl]-amide,
126 N-cyclopropyl-3-phenyl-N-[3-(4-pyrrolidin-1-yl-quinazolin-6-yl)-benzyl]-acrylamide,
127 4-chloro-N-cyclopropyl-N-[3-(4-pyrrolidin-1-yl-quinazolin-6-yl)-benzyl]-benzamide,
128 4-cyano-N-cyclopropyl-N-[3-(4-pyrrolidin-1-yl-quinazolin-6-yl)-benzyl]-benzamide,
129 pyrazine-2-carboxylic acid cyclopropyl-[3-(4-pyrrolidin-1-yl-quinazolin-6-yl)-benzyl]-amide,
130 6-bromo-pyridine-2-carboxylic acid cyclopropyl-[3-(4-pyrrolidin-1-yl-quinazolin-6-yl)-benzyl]-amide,
131 thiophene-3-carboxylic acid cyclopropyl-[3-(4-pyrrolidin-1-yl-quinazolin-6-yl)-benzyl]-amide,
132 6-chloro-N-cyclopropyl-N-[3-(4-pyrrolidin-1-yl-quinazolin-6-yl)-benzyl]-nicotinamide,
133 2-cyano-N-cyclopropyl-N-[3-(4-pyrrolidin-1-yl-quinazolin-6-yl)-benzyl]-acetamide,
134 N-cyclopropyl-4-methyl-N-[3-(4-pyrrolidin-1-yl-quinazolin-6-yl)-benzyl]-benzamide,
135 N-cyclopropyl-N-[3-(4-pyrrolidin-1-yl-quinazolin-6-yl)-benzyl]-2-trifluoromethyl-benzamide,
136 5-bromo-thiophene-2-carboxylic acid cyclopropyl-[3-(4-pyrrolidin-1-yl-quinazolin-6-yl)-benzyl]-amide,
137 N-cyclopropyl-N-[3-(4-pyrrolidin-1-yl-quinazolin-6-yl)-benzyl]-isonicotinamide,
138 N-cyclopropyl-N-[3-(4-pyrrolidin-1-yl-quinazolin-6-yl)-benzyl]-butyramide,
139 N-cyclopropyl-2-fluoro-N-[3-(4-pyrrolidin-1-yl-quinazolin-6-yl)-benzyl]-4-trifluoromethyl-benzamide,
140 thiophene-2-carboxylic acid cyclopropyl-[3-(4-pyrrolidin-1-yl-quinazolin-6-yl)-benzyl]-amide,
141 N-cyclopropyl-N-[3-(4-pyrrolidin-1-yl-quinazolin-6-yl)-benzyl]-nicotinamide,
142 N-cyclopropyl-4-methoxy-N-[3-(4-pyrrolidin-1-yl-quinazolin-6-yl)-benzyl]-benzamide,
143 2-chloro-N-cyclopropyl-N-[3-(4-pyrrolidin-1-yl-quinazolin-6-yl)-benzyl]-benzamide,
144 furan-2-carboxylic acid cyclopropyl-[3-(4-pyrrolidin-1-yl-quinazolin-6-yl)-benzyl]-amide,
145 N-cyclopropyl-2-phenoxy-N-[3-(4-pyrrolidin-1-yl-quinazolin-6-yl)-benzyl]-acetamide,
146 3-cyano-N-cyclopropyl-N-[3-(4-pyrrolidin-1-yl-quinazolin-6-yl)-benzyl]-benzamide,
147 pyridine-2-carboxylic acid cyclopropyl-[3-(4-pyrrolidin-1-yl-quinazolin-6-yl)-benzyl]-amide,
148 N-benzyl-N-[3-(4-isopropylamino-quinazolin-6-yl)-benzyl]-butyramide,
149 furan-2-carboxylic acid benzyl-[3-(4-isopropylamino-quinazolin-6-yl)-benzyl]-amide,
150 N-benzyl-2-chloro-N-[3-(4-isopropylamino-quinazolin-6-yl)-benzyl]-benzamide,
151 N-benzyl-N-[3-(4-isopropylamino-quinazolin-6-yl)-benzyl]-isonicotinamide,
152 N-benzyl-N-[3-(4-cyclopropylamino-quinazolin-6-yl)-benzyl]-2-fluoro-4-trifluoromethyl-benzamide,
153 N-benzyl-N-[3-(4-isopropylamino-quinazolin-6-yl)-benzyl]-nicotinamide,
154 thiophene-2-carboxylic acid benzyl-[3-(4-isopropylamino-quinazolin-6-yl)-benzyl]-amide,
155 N-benzyl-N-[3-(4-isopropylamino-quinazolin-6-yl)-benzyl]-4-methoxy-benzamide,
156 N-cyclopropyl-N-[3-(4-cyclopropylamino-quinazolin-6-yl)-benzyl]-2-hydroxy-benzamide,
157 2-bromo-N-cyclopropyl-N-[3-(4-cyclopropylamino-quinazolin-6-yl)-benzyl]-benzamide,
158 N-cyclopropyl-N-[3-(4-cyclopropylamino-quinazolin-6-yl)-benzyl]-3,4-dimethoxy-benzamide,
159 isoquinoline-3-carboxylic acid cyclopropyl-[3-(4-cyclopropylamino-quinazolin-6-yl)-benzyl]-amide,
160 4-chloro-N-cyclopropyl-N-[3-(4-cyclopropylamino-quinazolin-6-yl)-benzyl]-benzamide,
161 4-cyano-N-cyclopropyl-N-[3-(4-cyclopropylamino-quinazolin-6-yl)-benzyl]-benzamide,
162 pyrazine-2-carboxylic acid cyclopropyl-[3-(4-cyclopropylamino-quinazolin-6-yl)-benzyl]-amide,
163 6-bromo-pyridine-2-carboxylic acid cyclopropyl-[3-(4-cyclopropylamino-quinazolin-6-yl)-benzyl]-amide,
164 N-cyclopropyl-N-[3-(4-cyclopropylamino-quinazolin-6-yl)-benzyl]-2-phenoxy-acetamide,
165 N-cyclopropyl-N-[3-(4-cyclopropylamino-quinazolin-6-yl)-benzyl]-isonicotinamide,
166 5-bromo-thiophene-2-carboxylic acid cyclopropyl-[3-(4-cyclopropylamino-quinazolin-6-yl)-benzyl]-amide,
167 N-cyclopropyl-N-[3-(4-cyclopropylamino-quinazolin-6-yl)-benzyl]-2-trifluoromethyl-benzamide,
168 N-cyclopropyl-N-[3-(4-cyclopropylamino-quinazolin-6-yl)-benzyl]-butyramide,
169 furan-2-carboxylic acid cyclopropyl-[3-(4-cyclopropylamino-quinazolin-6-yl)-benzyl]-amide,
170 pyridine-2-carboxylic acid cyclopropyl-[3-(4-cyclopropylamino-quinazolin-6-yl)-benzyl]-amide,
171 N-cyclopropyl-N-[3-(4-cyclopropylamino-quinazolin-6-yl)-benzyl]-4-methoxy-benzamide,
172 2-chloro-N-cyclopropyl-N-[3-(4-cyclopropylamino-quinazolin-6-yl)-benzyl]-benzamide,
173 N-cyclopropyl-N-[3-(4-cyclopropylamino-quinazolin-6-yl)-benzyl]-2-dimethylamino-acetamide,
174 N-cyclopropyl-N-[3-(4-cyclopropylamino-quinazolin-6-yl)-benzyl]-2-pyridin-2-yl-acetamide,
175 N-[3-(4-cyclopropylamino-quinazolin-6-yl)-benzyl]-4,N-dimethyl-benzamide,
176 3-cyano-N-cyclopropyl-N-[3-(4-cyclopropylamino-quinazolin-6-yl)-benzyl]-benzamide,
177 N-cyclopropyl-N-[3-(4-cyclopropylamino-quinazolin-6-yl)-benzyl]-2-fluoro-4-trifluoromethyl-benzamide,
178 thiophene-2-carboxylic acid cyclopropyl-[3-(4-cyclopropylamino-quinazolin-6-yl)-benzyl]-amide,
179 6-chloro-N-cyclopropyl-N-[3-(4-cyclopropylamino-quinazolin-6-yl)-benzyl]-nicotinamide,
180 1-[3-(4-cyclopropylamino-2-methyl-quinazolin-6-yl)-benzyl]-pyrrolidine-2,5-dione,
181 1-[3-(4-cyclopropylamino-2-trifluoromethyl-quinazolin-6-yl)-benzyl]-pyrrolidine-2,5-dione,
182 1-[3-(2,4-bis-cyclopropylamino-quinazolin-6-yl)-benzyl]-pyrrolidine-2,5-dione,
183 1-[3-(2-amino-4-cyclopropylamino-quinazolin-6-yl)-benzyl]-pyrrolidine-2,5-dione,
184 1-[3-(4-cyclopropylamino-2-methylamino-quinazolin-6-yl)-benzyl]-pyrrolidine-2,5-dione,
185 1-[3-(4-cyclopropylamino-2-phenylamino-quinazolin-6-yl)-benzyl]-pyrrolidine-2,5-dione,
186 1-[3-(4-cyclopropylamino-2-dimethylamino-quinazolin-6-yl)-benzyl]-pyrrolidine-2,5-dione,
187 1-[3-(2-tert-butyl-4-cyclopropylamino-quinazolin-6-yl)-benzyl]-pyrrolidine-2,5-dione,
188 N-tert-butyl-2-cyano-N-(5-(4-(cyclopropylamino)quinazolin-6-yl)-2-fluorobenzyl)acetamide,
189 4-(5-(4-(cyclopropylamino)quinazolin-6-yl)-2-fluorobenzyl)morpholine-3,5-dione,
190 methyl 2-(2-cyano-N-(3-(4-(cyclopropylamino)quinazolin-6-yl)benzyl)acetamido)acetate,
191 N-cyclopropyl-N-(3-(4-(cyclopropylamino)quinazolin-6-yl)-2-fluorobenzyl)acetamide,
192 1-(5-(4-(cyclopropylamino)quinazolin-6-yl)-2-fluorobenzyl)-1H-pyrrole-2,5-dione,
193 1-(5-(4-(cyclopropylamino)quinazolin-6-yl)-2-fluorobenzyl)pyrrolidine-2,5-dione,
194 1-[3-(4-cyclopropylamino-quinazolin-6-yl)-benzyl]-piperidine-2,6-dione,
195 2-cyano-N-[3-(4-cyclopropylamino-quinazolin-6-yl)-benzyl]-N-pyridin-3-yl-acetamide,
196 1-[3-(4-tert-butylamino-quinazolin-6-yl)-benzyl]-pyrrolidine-2,5-dione hydrochloride,
197 2-cyano-N-cyclopropyl-N-[3-(4-cyclopropylamino-quinazolin-6-yl)-2-fluoro-benzyl]-acetamide,
198 1-[3-(4-methylamino-quinazolin-6-yl)-benzyl]-pyrrolidine-2,5-dione,
199 1-(3-(4-(benzylamino)quinazolin-6-yl)benzyl)pyrrolidine-2,5-dione,
200 N-cyanomethyl-N-[3-(4-cyclopropylamino-quinazolin-6-yl)-benzyl]-acetamide,
201 N-[5-(4-tert-butylamino-quinazolin-6-yl)-2-fluoro-benzyl]-2-cyano-N-cyclopropyl-acetamide,
202 N-tert-butyl-N-[3-(4-cyclopropylamino-quinazolin-6-yl)-benzyl]-acetamide,
203 {acetyl-[3-(4-cyclopropylamino-quinazolin-6-yl)-benzyl]-amino}-methyl acetate,
204 tert-butyl 2-(N-(3-(4-(cyclopropylamino)quinazolin-6-yl)benzyl)acetamido) ethylcarbamate,
205 N-cyclopropyl-N-[3-(4-cyclopropylamino-quinazolin-6-yl)-benzyl]-3,3,3-trifluoro-propionamide; hydrochloride,
206 cyclopropyl-{6-[3-(1,1-dioxo-1λ⁶-isothiazolidin-2-yl methyl)-phenyl]-quinazoline-4-yl}-amine; hydrochloride,
207 N-(3-(4-(cyclopropylamino)quinazolin-6-yl)benzyl)-N-phenylacetamide hydrochloride,
208 N-cyclopropyl-N-[3-(4-cyclopropylamino-quinazolin-6-yl)-benzyl]-2-fluoro-acetamide,
209 N-cyclopentyl-N-(3-(4-(cyclopropylamino)quinazolin-6-yl)benzyl)acetamide,
210 N-cyclopentyl-N-(3-(4-(cyclopropylamino)quinazolin-6-yl)benzyl)methane sulfonamide,
211 2-cyano-N-cyclopropyl-N-[3-(4-cyclopropylamino-quinazolin-6-yl)-benzyl]-acetamide,
212 4-(3-(4-(cyclopropylamino)quinazolin-6-yl)benzyl)morpholine-3,5-dione,
213 N-(3-(4-(cyclopropylamino)quinazolin-6-yl)benzyl)-N-(2-methoxyethyl)acetamide,
214 N-[3-(4-cyclopropylamino-quinazolin-6-yl)-benzyl]-N-phenethyl-acetamide,
215 N-(3-(4-(cyclopropylamino)quinazolin-6-yl)benzyl)-N-(furan-2-yl-methyl)methane sulfonamide,
216 N-[3-(4-cyclopropylamino-quinazolin-6-yl)-benzyl]-N-furan-2-yl-methyl-acetamide,
217 1-[3-(4-cyclopropylamino-quinazolin-6-yl)-benzyl]-pyrrolidine-2-one; hydrochloride,
218 N-butyl-N-[3-(4-cyclopropylamino-quinazolin-6-yl)-benzyl]-acetamide; hydrochloride,
219 N-benzyl-N-[3-(4-cyclopropylamino-quinazolin-6-yl)-benzyl]-acetamide; hydrochloride,
220 1-[3-(4-cyclopropylamino-quinazolin-6-yl)-benzyl]-3-phenyl-piperidine-2,6-dione; hydrochloride,
221 N-[3-(4-cyclopropylamino-quinazolin-6-yl)-benzyl]-N-methyl-acetamide,
222 N-benzyl-N-(3-(4-(cyclopropylamino)quinazolin-6-yl)benzyl)methane sulfonamide,
223 N-(3-(4-(cyclopropylamino)quinazolin-6-yl)benzyl)-N-methyl methane sulfonamide,
224 1-[3-(4-cyclopropylamino-quinazolin-6-yl)-benzyl]-4-methyl-piperidine-2,6-dione; hydrochloride,
225 N-cyclopropyl-N-(3-(4-(cyclopropylamino)quinazolin-6-yl)benzyl)-2,2,2-trifluoroethane sulfonamide,
226 N-cyclopropyl-N-(3-(4-(cyclopropylamino)quinazolin-6-yl)benzyl)-4-(trifluoromethyl)benzole sulfonamide,
227 N-cyclopropyl-N-(3-(4-(cyclopropylamino)quinazolin-6-yl)benzyl)benzole sulfonamide,
228 2-[3-(4-cyclopropylamino-quinazolin-6-yl)-benzyl]-hexahydro-isoindole-1,3-dione; hydrochloride,
229 1-[3-(4-cyclopropylamino-quinazolin-6-yl)-benzyl]-3-phenyl-pyrrolidine-2,5-dione; hydrochloride,
230 N-cyclopropyl-N-[5-(4-cyclopropylamino-quinazolin-6-yl)-pyridin-3-yl-methyl]-benzamide; hydrochloride,
231 N-cyclopropyl-N-(1-(3-(4-(cyclopropylamino)quinazolin-6-yl)phenyl)ethyl)methane sulfonamide,
232 N-cyclopropyl-N-[5-(4-cyclopropylamino-quinazolin-6-yl)-furan-2-yl-methyl]-acetamide,
233 N-cyclopropyl-N-{1-[3-(4-cyclopropylamino-quinazolin-6-yl)-phenyl]-ethyl}-benzamide,
234 N-cyclopropyl-N-[5-(4-cyclopropylamino-quinazolin-6-yl)-pyridin-3-yl-methyl]-methane sulfonamide,
235 N-cyclopropyl-N-{1-[3-(4-cyclopropylamino-quinazolin-6-yl)-phenyl]-ethyl}-acetamide,
236 N-{1-[3-(4-cyclopropylamino-quinazolin-6-yl)-benzyl]-2,5-dioxo-pyrrolidine-3-yl}-2,2,2-trifluoro-acetamide; hydrochloride,
237 N-cyclopropyl-N-[3-(4-cyclopropylamino-quinazolin-6-yl)-benzyl]-methyl malonate,
238 N-cyclopropyl-N-[3-(4-cyclopropylamino-quinazolin-6-yl)-benzyl]-methyl oxalate,
239 N-cyclopropyl-N-[3-(4-cyclopropylamino-quinazolin-6-yl)-5-methyl-benzyl]-acetamide,
240 N-cyclopropyl-N-[5-(4-cyclopropylamino-quinazolin-6-yl)-pyridin-3-yl-methyl]-acetamide,
241 2-[3-(4-cyclopropylamino-quinazolin-6-yl)-benzyl]-isoindole-1,3-dione; hydrochloride,
242 2-[3-(4-cyclopropylamino-quinazolin-6-yl)-benzyl]-hexahydro-isoindole-1,3-dione hydrochloride,
243 8-[3-(4-cyclopropylamino-quinazolin-6-yl)-benzyl]-8-aza-spiro[4.5]decane-7,9-dione; hydrochloride,
244 N-cyclopropyl-N-[3-(4-cyclopropylamino-quinazolin-6-yl)-2,6-difluoro-benzyl]-benzamide,
245 1-[3-(4-cyclopropylamino-quinazolin-6-yl)-benzyl]-3-methyl-pyrrolidine-2,5-dione,
246 N-cyclopropyl-N-(3-(4-(cyclopropylamino)quinazolin-6-yl)-2,6-difluorobenzyl)methane sulfonamide,
247 1-cyclopropyl-1-[3-(4-cyclopropylamino-quinazolin-6-yl)-benzyl]-3-isopropyl-urea,
248 N-cyclopropyl-N-[3-(4-cyclopropylamino-quinazolin-6-yl)-benzyl]-nicotinamide,
249 N-cyclopropyl-N-[3-(4-cyclopropylamino-quinazolin-6-yl)-2,6-difluoro-benzyl]-acetamide,
250 N-cyclopropyl-N-(3-(4-(cyclopropylamino)quinazolin-6-yl)-4-methoxybenzyl)methane sulfonamide,
251 N-cyclopropyl-N-(5-(4-(cyclopropylamino)quinazolin-6-yl)-2-fluorobenzyl)methane sulfonamide,
252 N-cyclopropyl-N-[3-(4-cyclopropylamino-quinazolin-6-yl)-benzyl]-2-methoxy-acetamide,
253 isoxazole-5-carboxylic acid cyclopropyl-[3-(4-cyclopropylamino-quinazolin-6-yl)-benzyl]-amide,
254 N-cyclopropyl-N-[3-(4-cyclopropylamino-quinazolin-6-yl)-benzyl]-2,3,4,5,6-pentafluoro-benzamide,
255 N-cyclopropyl-N-[3-(4-cyclopropylamino-quinazolin-6-yl)-benzyl]-3-phenyl-acrylamide,
256 pentanoic acid cyclopropyl-[3-(4-cyclopropylamino-quinazolin-6-yl)-benzyl]-amide,
257 N-cyclopropyl-N-[3-(4-cyclopropylamino-quinazolin-6-yl)-benzyl]-isonicotinamide,
258 1-[3-(4-cyclopropylamino-quinazolin-6-yl)-benzyl]-pyrrolidine-2,5-dione,
259 N-cyclopropyl-N-[5-(4-cyclopropylamino-quinazolin-6-yl)-2-methoxy-benzyl]-acetamide,
260 N-cyclopropyl-N-[5-(4-cyclopropylamino-quinazolin-6-yl)-2-fluoro-benzyl]-acetamide,
261 N-cyclopropyl-N-[3-(4-cyclopropylamino-quinazolin-6-yl)-4-methoxy-benzyl]-acetamide,
262 N-cyclopropyl-N-[4-(4-cyclopropylamino-quinazolin-6-yl)-thiophen-2-ylmethyl]-acetamide,
263 N-cyclopropyl-N-[3-(4-cyclopropylamino-quinazolin-6-yl)-benzyl]-3-phenyl-propionamide,
264 N-cyclopropyl-N-[3-(4-cyclopropylamino-quinazolin-6-yl)-benzyl]-2-phenyl-acetamide,
265 N-cyclopropyl-N-(3-(4-(cyclopropylamino)quinazolin-6-yl)benzyl)cyclohexane carboxamide,
266 N-cyclopropyl-N-(3-(4-(cyclopropylamino)quinazolin-6-yl)benzyl)pivalamide,
267 N-cyclopropyl-N-(3-(4-(cyclopropylamino)quinazolin-6-yl)-4-methoxybenzyl)benzamide hydrochloride,
268 N-cyclopropyl-N-(3-(4-(cyclopropylamino)quinazolin-6-yl)-4-methoxybenzyl)benzamide,
269 N-cyclopropyl-N-(5-(4-(cyclopropylamino)quinazolin-6-yl)-2-fluorobenzyl)benzamide,
270 N-cyclopropyl-N-(3-(4-(cyclopropylamino)quinazolin-6-yl)benzyl)methane sulfonamide,
271 N-cyclopropyl-N-(3-(4-(cyclopropylamino)quinazolin-6-yl)benzyl)acetamide,
272 N-cyclopropyl-N-(3-(4-(cyclopropyl(methyl)amino)quinazolin-6-yl)benzyl)picolinamide,
273 N-cyclopropyl-N-(3-(4-(cyclopropylamino)quinazolin-6-yl)benzyl)-2,2,2-trifluoroacetamide,
274 N-cyclopropyl-N-(3-(4-(cyclopropylamino)quinazolin-6-yl)benzyl)benzamide,
275 N-cyclopropyl-N-(3-(4-(cyclopropylamino)quinazolin-6-yl)benzyl) picolinamide,
276 1-(3-(4-(benzylamino)quinazolin-6-yl)benzyl)pyrrolidine-2,5-dione,
277 2-cyano-N-cyclopropyl-N-(5-(4-(cyclopropylamino)quinazolin-6-yl)-2-fluorobenzyl)acetamide,
278 1-(5-(4-(cyclopropylamino)quinazolin-6-yl)-2-fluorobenzyl)piperidine-2,6-dione,
279 N-tert-butyl-N-(5-(4-(cyclopropylamino)quinazolin-6-yl)-2-fluorobenzyl)acetamide, tert-butyl
280 2-(2-cyano-N-(3-(4-(cyclopropylamino)quinazolin-6-yl)benzyl)acetamido)ethylcarbamate,
281 2-cyano-N-cyclopropyl-N-(5-(4-(cyclopropylamino)quinazolin-6-yl)-2-fluorobenzyl)acetamide hydrochloride,
282 N-cyclopropyl-N-(5-(4-(cyclopropylamino)quinazolin-6-yl)-2-fluorobenzyl)-3,3,3-trifluoropropanamide,
283 2-chloro-N-cyclopropyl-N-(5-(4-(cyclopropylamino)quinazolin-6-yl)-2-fluorobenzyl)acetamide,
284 N-(2-aminoethyl)-2-cyano-N-(3-(4-(cyclopropylamino)quinazolin-6-yl)benzyl)acetamide hydrochloride,
285 1-(3-(4-(dimethylamino)quinazolin-6-yl)benzyl)pyrrolidine-2,5-dione hydrochloride,
286 1-(3-(4-(butylamino)quinazolin-6-yl)benzyl)pyrrolidine-2,5-dione hydrochloride,
287 1-(3-(4-(cyclopentylamino)quinazolin-6-yl)benzyl)pyrrolidine-2,5-dione hydrochloride,
288 1-(3-(2-benzyl-4-(cyclopropylamino)quinazolin-6-yl)benzyl)pyrrolidine-2,5-dione,
289 1-(3-(4-(cyclopropylamino)-2-(pyridin-2-yl)quinazolin-6-yl)benzyl)pyrrolidine-2,5-dione,
290 1-(3-(4-(cyclopropylamino)-2-morpholinoquinazolin-6-yl)benzyl)pyrrolidine-2,5-dione,
291 1-(3-(2-(benzylamino)-4-(cyclopropylamino)quinazolin-6-yl)benzyl)pyrrolidine-2,5-dione,
292 N-cyclopropyl-6-(3-(indolin-1-ylmethyl)phenyl)quinazoline-4-amine,
293 1-(3-(4-(cyclopropylamino)-2-phenylquinazolin-6-yl)benzyl)pyrrolidine-2,5-dione,
294 N-cyclopropyl-6-(3-((cyclopropylamino)methyl)-phenyl)quinazoline-4-amine,
295 tert-butyl 3-(4-(cyclopropylamino)quinazolin-6-yl)benzylcarbamate,
296 N-cyclopropyl-6-(3-((dimethylamino)methyl)phenyl)quinazoline-4-amine,
297 6-(3-(aminomethyl)phenyl)-N-cyclopropylquinazoline-4-amine dihydrochloride,
298 N-cyclopropyl-6-(3-((cyclopropylamino)methyl)-4-methoxyphenyl)quinazoline-4-amine,
299 N-cyclopropyl-6-(3-((cyclopropylamino)methyl)-4-fluorophenyl)quinazoline-4-amine,
300 N-cyclopropyl-6-(5-((cyclopropylamino)methyl)thiophen-3-yl)quinazoline-4-amine,
301 N-cyclopropyl-6-(5-((cyclopropylamino)methyl)-2-methoxyphenyl)quinazoline-4-amine,
302 N-(3-(4-(cyclopropylamino)quinazolin-6-yl)benzyl)acetamide,
303 N-(3-(4-(cyclopropylamino)quinazolin-6-yl)benzyl)benzamide,
304 N-cyclopropyl-6-(3-((cyclopropylamino)methyl)-2,4-difluorophenyl)quinazoline-4-amine,
305 N-cyclopropyl-6-(5-((cyclopropylamino)methyl)pyridin-3-yl)quinazoline-4-amine,
306 N-cyclopropyl-6-(3-((cyclopropylamino)methyl)-5-methylphenyl)quinazoline-4-amine,
307 N-cyclopropyl-6-(3-(1-(cyclopropylamino)ethyl)phenyl)quinazoline-4-amine,
308 6-(3-((benzylamino)methyl)phenyl)-N-cyclopropylquinazoline-4-amine,
309 N-cyclopropyl-6-(3-((methylamino)methyl)phenyl)quinazoline-4-amine,
310 6-(3-((butylamino)methyl)phenyl)-N-cyclopropylquinazoline-4-amine,
311 N-(6-(3-(acetamidomethyl)phenyl)quinazoline-4-yl)-N-cyclopropylacetamide hydrochloride,
312 N-cyclopropyl-6-(3-((furan-2-ylmethylamino)methyl)phenyl)quinazoline-4-amine dihydrochloride,
313 N-cyclopropyl-6-(3-((phenethylamino)methyl)phenyl)quinazoline-4-amine,
314 N-cyclopropyl-6-(3-((2-methoxyethylamino)methyl)phenyl)quinazoline-4-amine dihydrochloride,
315 6-(3-((2-chloroethylamino)methyl)phenyl)-N-cyclopropylquinazoline-4-amine,
316 3-chloro-N-(3-(4-(cyclopropylamino)quinazolin-6-yl)benzyl)propane-1-sulfonamide hydrochloride,
317 5-chloro-N-(3-(4-(cyclopropylamino)quinazolin-6-yl)benzyl)pentanamide hydrochloride,
318 6-(3-((cyclopentylamino)methyl)phenyl)-N-cyclopropylquinazoline-4-amine,
319 N-cyclopropyl-6-(3-((phenylamino)methyl)phenyl)quinazoline-4-amine,
320 2-(3-(4-(cyclopropylamino)quinazolin-6-yl)benzylamino)acetonitrile,
321 methyl 2-(3-(4-(cyclopropylamino)quinazolin-6-yl)benzylamino)acetate,
322 N-tert-butyl-6-(3-((cyclopropylamino)methyl)-4-fluorophenyl)quinazoline-4-amine,
323 6-(3-((tert-butylamino)methyl)phenyl)-N-cyclopropylquinazoline-4-amine,
324 N-cyclopropyl-6-(3-((cyclopropylamino)methyl)-2-fluorophenyl)quinazoline-4-amine,
325 N-cyclopropyl-6-(3-((pyridin-3-ylamino)methyl)phenyl)quinazoline-4-amine,
326 tert-butyl 2-(3-(4-(cyclopropylamino)quinazolin-6-yl)benzylamino) ethylcarbamate,
327 6-(3-((tert-butylamino)methyl)-4-fluorophenyl)-N-cyclopropylquinazoline-4-amine,
328 2-(2-(3-(4-(cyclopropylamino)quinazolin-6-yl)benzylamino)-2-oxoethyl)benzoic acid,
329 4-oxo-4-(3-(4-(phenylamino)quinazolin-6-yl)benzylamino)butyric acid,
330 4-(3-(4-(carboxymethylamino)quinazolin-6-yl)benzylamino)-4-oxobutyric acid;
331 N-benzyl-5-bromo-N-(3-(4-(isopropylamino)quinazolin-6-yl)benzyl)thiophene-2-carboxamide,
332 N-benzyl-N-(3-(4-(isopropylamino)quinazolin-6-yl)benzyl)picolinamide,
333 N-(3-(4-(cyclopropylamino)quinazolin-6-yl)benzyl)picolinamide,
334 2-chloro-N-(3-(4-(cyclopropylamino)quinazolin-6-yl)benzyl)benzamide,
335 N-(3-(4-(cyclopropylamino)quinazolin-6-yl)benzyl)-4-methoxybenzamide,
336 N-(3-(4-(cyclopropylamino)quinazolin-6-yl)benzyl)thiophene-2-carboxamide,
337 N-(3-(4-(cyclopropylamino)quinazolin-6-yl)benzyl)-2-fluoro-4-(trifluoromethyl)benzamide,
338 N-(3-(4-(cyclopropylamino)quinazolin-6-yl)benzyl)butyramide,
339 5-bromo-N-(3-(4-(cyclopropylamino)quinazolin-6-yl)benzyl)thiophene-2-carboxamide,
340 N-(3-(4-(cyclopropylamino)quinazolin-6-yl)benzyl)-2-(trifluoromethyl)benzamide,
341 N-(3-(4-(cyclopropylamino)quinazolin-6-yl)benzyl)-4-methylbenzamide,
342 6-chloro-N-(3-(4-(cyclopropylamino)quinazolin-6-yl)benzyl)nicotinamide,
343 N-(3-(4-(cyclopropylamino)quinazolin-6-yl)benzyl)thiophene-3-carboxamide,
344 N-(3-(4-(cyclopropylamino)quinazolin-6-yl)benzyl)-2-phenoxyacetamide,
345 6-bromo-N-(3-(4-(cyclopropylamino)quinazolin-6-yl)benzyl)picolinamide,
346 4-cyano-N-(3-(4-(cyclopropylamino)quinazolin-6-yl)benzyl)benzamide,
347 N-(3-(4-(cyclopropylamino)quinazolin-6-yl)benzyl)cinnamamide,
348 N-(3-(4-(cyclopropylamino)quinazolin-6-yl)benzyl)isoquinoline-3-carboxamide,
349 N-(3-(4-(cyclopropylamino)quinazolin-6-yl)benzyl)-2,4-difluorobenzamide,
350 2-bromo-N-(3-(4-(cyclopropylamino)quinazolin-6-yl)benzyl)benzamide,
351 N-(3-(4-(cyclopropylamino)quinazolin-6-yl)benzyl)furan-2-carboxamide,
352 N-(3-(4-(cyclopropylamino)quinazolin-6-yl)benzyl)-2-(dimethylamino)acetamide,
353 N-(3-(4-(cyclopropylamino)quinazolin-6-yl)benzyl)nicotinamide,
354 N-(3-(4-(cyclopropylamino)quinazolin-6-yl)benzyl)-2-hydroxybenzamide,
355 N-(3-(4-(cyclopropylamino)quinazolin-6-yl)benzyl)isonicotinamide,
356 2-cyano-N-(3-(4-(cyclopropylamino)quinazolin-6-yl)benzyl)acetamide,
357 N-(3-(4-(cyclopropylamino)quinazolin-6-yl)benzyl)-3-methylthiophene-2-carboxamide,
358 3-cyano-N-(3-(4-(cyclopropylamino)quinazolin-6-yl)benzyl)benzamide,
359 N-(3-(4-(cyclopropylamino)quinazolin-6-yl)benzyl)-2-(pyridin-2-yl)acetamide,
360 N-(3-(4-(cyclopropylamino)quinazolin-6-yl)benzyl)pyrazine-2-carboxamide,
361 4-chloro-N-(3-(4-(cyclopropylamino)quinazolin-6-yl)benzyl)benzamide,
362 N-(3-(4-(cyclopropylamino)quinazolin-6-yl)benzyl)-3,4-dimethoxybenzamide,
363 N-cyclopropyl-N-(3-(4-(3-methylbenzylamino)quinazolin-6-yl)benzyl)benzole sulfonamide,
364 N-cyclopropyl-2-fluoro-N-(3-(4-(3-methylbenzylamino)quinazolin-6-yl)benzyl)-4-(trifluoromethyl)benzamide,
365 N-cyclopropyl-N-(3-(4-(3-methylbenzylamino)quinazolin-6-yl)benzyl)-2-phenylacetamide,
366 N-cyclopropyl-N-(5-(4-(cyclopropylamino)quinazolin-6-yl)-2-fluorobenzyl)-3,4-dimethoxybenzamide,
367 N-cyclopropyl-N-(5-(4-(cyclopropylamino)quinazolin-6-yl)-2-fluorobenzyl)nicotinamide,
368 N-cyclopropyl-N-(5-(4-(cyclopropylamino)quinazolin-6-yl)-2-fluorobenzyl)benzole sulfonamide,
369 N-cyclopropyl-1,3-dimethyl-N-(3-(4-(phenethylamino)quinazolin-6-yl)benzyl)-1H-pyrazole-5-carboxamide,
370 N-cyclopropyl-N-(3-(4-(phenethylamino)quinazolin-6-yl)benzyl)picolinamide,
371 N-cyclopropyl-4-methoxy-N-(3-(4-(3-methylbenzylamino)quinazolin-6-yl)benzyl)benzole sulfonamide,
372 N-cyclopropyl-N-(3-(4-(3-methylbenzylamino)quinazolin-6-yl)benzyl)-3-(trifluoromethyl)benzamide,
373 N-cyclopropyl-N-(3-(4-(3-methylbenzylamino)quinazolin-6-yl)benzyl)-3-phenylpropanamide,
374 N-cyclopropyl-N-(5-(4-(cyclopropylamino)quinazolin-6-yl)-2-fluorobenzyl)-3-fluorobenzamide,
375 N-cyclopropyl-N-(5-(4-(cyclopropylamino)quinazolin-6-yl)-2-fluorobenzyl)-3-(trifluoromethyl)benzole sulfonamide,
376 N-cyclopropyl-N-(3-(4-(phenethylamino)quinazolin-6-yl)benzyl)benzamide,
377 N-cyclopropyl-N-(3-(4-(phenethylamino)quinazolin-6-yl)benzyl)isonicotinamide,
378 N-cyclopropyl-N-(3-(4-(3-methylbenzylamino)quinazolin-6-yl)benzyl)thiophene-2-sulfonamide,
379 N-cyclopropyl-N-(3-(4-(3-methylbenzylamino)quinazolin-6-yl)benzyl)-2-(trifluoromethyl)benzamide,
380 N-cyclopropyl-N-(3-(4-(3-methylbenzylamino)quinazolin-6-yl)benzyl)methane sulfonamide,
381 N-cyclopropyl-N-(5-(4-(cyclopropylamino)quinazolin-6-yl)-2-fluorobenzyl)-2,4-difluorobenzamide,
382 N-cyclopropyl-N-(5-(4-(cyclopropylamino)quinazolin-6-yl)-2-fluorobenzyl)pentanamide,
383 N-cyclopropyl-N-(5-(4-(cyclopropylamino)quinazolin-6-yl)-2-fluorobenzyl)-4-methoxybenzole sulfonamide
384 N-cyclopropyl-3,4-dimethoxy-N-(3-(4-(phenethylamino)quinazolin-6-yl)benzyl)benzamide,
385 N-cyclopropyl-N-(3-(4-(phenethylamino)quinazolin-6-yl)benzyl)nicotinamide,
386 N-cyclopropyl-N-(3-(4-(3-methylbenzylamino)quinazolin-6-yl)benzyl)furan-2-carboxamide,
387 3,5-dichloro-N-cyclopropyl-N-(3-(4-(3-methylbenzylamino)quinazolin-6-yl)benzyl)benzamide,
388 N-cyclopropyl-N-(3-(4-(3-methylbenzylamino)quinazolin-6-yl)benzyl) 1-phenylmethane sulfonamide,
389 N-cyclopropyl-N-(5-(4-(cyclopropylamino)quinazolin-6-yl)-2-fluorobenzyl)-2,3,4,5,6-pentafluorobenzamide,
390 N-cyclopropyl-N-(5-(4-(cyclopropylamino)quinazolin-6-yl)-2-fluorobenzyl)-2-methoxyacetamide,
391 2,4-dichloro-N-cyclopropyl-N-(5-(4-(cyclopropylamino)quinazolin-6-yl)-2-fluorobenzyl)benzole sulfonamide,
392 N-cyclopropyl-3-fluoro-N-(3-(4-(phenethylamino)quinazolin-6-yl)benzyl)benzamide,
393 N-cyclopropyl-N-(3-(4-(phenethylamino)quinazolin-6-yl)benzyl)acetamide,
394 N-cyclopropyl-N-(3-(4-(3-methylbenzylamino)quinazolin-6-yl)benzyl)thiophene-2-carboxamide,
395 N-cyclopropyl-N-(3-(4-(3-methylbenzylamino)quinazolin-6-yl)benzyl)picolinamide,
396 2,4-dichloro-N-cyclopropyl-N-(3-(4-(3-methylbenzylamino)quinazolin-6-yl)benzyl)benzole sulfonamide,
397 N-cyclopropyl-N-(5-(4-(cyclopropylamino)quinazolin-6-yl)-2-fluorobenzyl)-2-fluoro-4-(trifluoromethyl)benzamide,
398 N-cyclopropyl-N-(5-(4-(cyclopropylamino)quinazolin-6-yl)-2-fluorobenzyl)-2-(dimethylamino)acetamide,
399 N-cyclopropyl-N-(5-(4-(cyclopropylamino)quinazolin-6-yl)-2-fluorobenzyl)-2,3,4,5,6-pentafluorobenzole sulfonamide,
400 N-cyclopropyl-2,4-difluoro-N-(3-(4-(phenethylamino)quinazolin-6-yl)benzyl)benzamide,
401 N-cyclopropyl-N-(3-(4-(phenethylamino)quinazolin-6-yl)benzyl)pivalamide,
402 N-cyclopropyl-1,3-dimethyl-N-(3-(4-(3-methylbenzylamino)quinazolin-6-yl)benzyl)-1H-pyrazole-5-carboxamide,
403 N-cyclopropyl-N-(3-(4-(3-methylbenzylamino)quinazolin-6-yl)benzyl)isonicotinamide,
404 N-cyclopropyl-2,3,4,5,6-pentafluoro-N-(3-(4-(3-methylbenzylamino)quinazolin-6-yl)benzyl)benzole sulfonamide,
405 N-cyclopropyl-N-(5-(4-(cyclopropylamino)quinazolin-6-yl)-2-fluorobenzyl)-3-(trifluoromethyl)benzamide,
406 N-cyclopropyl-N-(5-(4-(cyclopropylamino)quinazolin-6-yl)-2-fluorobenzyl)-2-phenylacetamide,
407 N-cyclopropyl-N-(5-(4-(cyclopropylamino)quinazolin-6-yl)-2-fluorobenzyl)thiophene-2-sulfonamide,
408 N-cyclopropyl-2,3,4,5,6-pentafluoro-N-(3-(4-(phenethylamino)quinazolin-6-yl)benzyl)benzamide,
409 N-cyclopropyl-N-(3-(4-(phenethylamino)quinazolin-6-yl)benzyl)pentanamide,
410 N-cyclopropyl-N-(3-(4-(3-methylbenzylamino)quinazolin-6-yl)benzyl)benzamide,
411 N-cyclopropyl-N-(3-(4-(3-methylbenzylamino)quinazolin-6-yl)benzyl)nicotinamide,
412 N-cyclopropyl-N-(3-(4-(3-methylbenzylamino)quinazolin-6-yl)benzyl)quinoline-8-sulfonamide,
413 N-cyclopropyl-N-(5-(4-(cyclopropylamino)quinazolin-6-yl)-2-fluorobenzyl)-2-(trifluoromethyl)benzamide,
414 N-cyclopropyl-N-(5-(4-(cyclopropylamino)quinazolin-6-yl)-2-fluorobenzyl)-3-phenylpropanamide,
415 N-cyclopropyl-N-(5-(4-(cyclopropylamino)quinazolin-6-yl)-2-fluorobenzyl)quinoline-8-sulfonamide,
416 N-cyclopropyl-2-fluoro-N-(3-(4-(phenethylamino)quinazolin-6-yl)benzyl)-4-(trifluoromethyl)benzamide,
417 N-cyclopropyl-2-(dimethylamino)-N-(3-(4-(phenethylamino)quinazolin-6-yl)benzyl)acetamide,
418 N-cyclopropyl-3,4-dimethoxy-N-(3-(4-(3-methylbenzylamino)quinazolin-6-yl)benzyl)benzamide,
419 N-cyclopropyl-N-(3-(4-(3-methylbenzylamino)quinazolin-6-yl)benzyl)acetamide,
420 N-cyclopropyl-N-(5-(4-(cyclopropyl(methyl)amino)quinazolin-6-yl)-2-fluorobenzyl)furan-2-carboxamide,
421 3,5-dichloro-N-cyclopropyl-N-(5-(4-(cyclopropylamino)quinazolin-6-yl)-2-fluorobenzyl)benzamide,
422 N-cyclopropyl-N-(3-(4-(phenethylamino)quinazolin-6-yl)benzyl)furan-2-carboxamide,
423 N-cyclopropyl-N-(3-(4-(phenethylamino)quinazolin-6-yl)benzyl)-3-(trifluoromethyl)benzamide,
424 N-cyclopropyl-N-(3-(4-(phenethylamino)quinazolin-6-yl)benzyl)-2-phenylacetamide,
425 N-cyclopropyl-3-fluoro-N-(3-(4-(3-methylbenzylamino)quinazolin-6-yl)benzyl)benzamide,
426 N-cyclopropyl-N-(3-(4-(3-methylbenzylamino)quinazolin-6-yl)benzyl)pentanamide,
427 N-cyclopropyl-N-(5-(4-(cyclopropyl(methyl)amino)quinazolin-6-yl)-2-fluorobenzyl)thiophene-2-carboxamide,
428 N-cyclopropyl-N-(5-(4-(cyclopropylamino)quinazolin-6-yl)-2-fluorobenzyl)picolinamide
429 N-cyclopropyl-N-(5-(4-(cyclopropylamino)quinazolin-6-yl)-2-fluorobenzyl)propane-2-sulfonamide,
430 N-cyclopropyl-N-(3-(4-(phenethylamino)quinazolin-6-yl)benzyl)thiophene-2-carboxamide,
431 N-cyclopropyl-N-(3-(4-(phenethylamino)quinazolin-6-yl)benzyl)-2-(trifluoromethyl)benzamide,
432 N-cyclopropyl-N-(3-(4-(phenethylamino)quinazolin-6-yl)benzyl)-3-phenylpropanamide,
433 N-cyclopropyl-2,4-difluoro-N-(3-(4-(3-methylbenzylamino)quinazolin-6-yl)benzyl)benzamide,
434 N-cyclopropyl-2-methoxy-N-(3-(4-(3-methylbenzylamino)quinazolin-6-yl)benzyl)acetamide,
435 N-cyclopropyl-N-(5-(4-(cyclopropylamino)quinazolin-6-yl)-2-fluorobenzyl)-1,3-dimethyl-1H-pyrazole-5-carboxamide,
436 N-cyclopropyl-N-(5-(4-(cyclopropylamino)quinazolin-6-yl)-2-fluorobenzyl)isonicotinamide,
437 N-cyclopropyl-N-(5-(4-(cyclopropylamino)quinazolin-6-yl)-2-fluorobenzyl)-1-phenylmethane sulfonamide,
438 N-cyclopropyl-N-(3-(4-(phenethylamino)quinazolin-6-yl)benzyl)isoxazole-5-carboxamide,
439 3,5-dichloro-N-cyclopropyl-N-(3-(4-(phenethylamino)quinazolin-6-yl)benzyl)benzamide,
440 N-cyclopropyl-N-(3-(4-(phenethylamino)quinazolin-6-yl)benzyl)methane sulfonamide,
441 N-cyclopropyl-N-(3-(4-(phenethylamino)quinazolin-6-yl)benzyl)-1-phenylmethane sulfonamide,
442 N-cyclopropyl-2,3,4,5,6-pentafluoro-N-(3-(4-(3-methylbenzylamino)quinazolin-6-yl)benzyl)benzamide,
443 2,3,4,5,6-pentafluoro-N-(3-(4-(methylamino)quinazolin-6-yl)benzyl)-N-(pyridin-2-ylmethyl)benzole sulfonamide,
444 N-(3-(4-(methylamino)quinazolin-6-yl)benzyl)-N-(pyridin-2-ylmethyl)-3-(trifluoromethyl)benzamide,
445 N-(3-(4-(methylamino)quinazolin-6-yl)benzyl)-3-phenyl-N-(pyridin-2-ylmethyl)propanamide,
446 N-(5-(4-(benzylamino)quinazolin-6-yl)-2-fluorobenzyl)-2,4-dichloro-N-methylbenzole sulfonamide,
447 N-cyclopropyl-N-(3-(4-(cyclopropylamino)-2-methylquinazolin-6-yl)benzyl)-2,3,4,5,6-pentafluorobenzamide,
448 2,4-dichloro-N-cyclopropyl-N-(3-(4-(cyclopropylamino)-2-methylquinazolin-6-yl)benzyl)benzole sulfonamide,
449 N-cyclopropyl-N-(3-(4-(phenethylamino)quinazolin-6-yl)benzyl)benzole sulfonamide,
450 N-(3-(4-(methylamino)quinazolin-6-yl)benzyl)-N-(pyridin-2-ylmethyl)quinoline-8-sulfonamide,
451 3,5-dichloro-N-(3-(4-(methylamino)quinazolin-6-yl)benzyl)-N-(pyridin-2-ylmethyl)benzamide,
452 N-cyclopropyl-N-(5-(4-(cyclopropylamino)quinazolin-6-yl)-2-fluorobenzyl)cyclohexane carboxamide,
453 N-(5-(4-(benzylamino)quinazolin-6-yl)-2-fluorobenzyl)-2,3,4,5,6-pentafluoro-N-methylbenzole sulfonamide,
454 N-cyclopropyl-N-(3-(4-(cyclopropylamino)-2-methylquinazolin-6-yl)benzyl)-2-fluoro-4-(trifluoromethyl)benzamide,
455 N-cyclopropyl-N-(3-(4-(cyclopropylamino)-2-methylquinazolin-6-yl)benzyl)-2,3,4,5,6-pentafluorobenzole sulfonamide,
456 N-cyclopropyl-N-(3-(4-(phenethylamino)quinazolin-6-yl)benzyl)-3-(trifluoromethyl)benzole sulfonamide,
457 N-cyclopropyl-N-(5-(4-(cyclopropylamino)quinazolin-6-yl)-2-fluorobenzyl)isoxazole-5-carboxamide,
458 N-(3-(4-(methylamino)quinazolin-6-yl)benzyl)-N-(pyridin-2-ylmethyl)furan-2-carboxamide,
459 N-(3-(4-(methylamino)quinazolin-6-yl)benzyl)-N-(pyridin-2-ylmethyl)picolinamide,
460 N-cyclopropyl-N-(3-(4-(phenethylamino)quinazolin-6-yl)benzyl)cyclohexane carboxamide,
461 N-(5-(4-(benzylamino)quinazolin-6-yl)-2-fluorobenzyl)-N-methylthiophene-2-sulfonamide,
462 N-cyclopropyl-N-(3-(4-(cyclopropylamino)-2-methylquinazolin-6-yl)benzyl)-3-(trifluoromethyl)benzamide,
463 N-cyclopropyl-N-(3-(4-(cyclopropylamino)-2-methylquinazolin-6-yl)benzyl)thiophene-2-sulfonamide,
464 N-cyclopropyl-4-methoxy-N-(3-(4-(phenethylamino)quinazolin-6-yl)benzyl)benzole sulfonamide,
465 N-cyclopropyl-N-(3-(4-(3-methylbenzylamino)quinazolin-6-yl)benzyl)pivalamide,
466 N-(3-(4-(methylamino)quinazolin-6-yl)benzyl)-N-(pyridin-2-ylmethyl)thiophene-2-carboxamide,
467 N-(3-(4-(methylamino)quinazolin-6-yl)benzyl)-N-(pyridin-2-ylmethyl)isonicotinamide,
468 N-cyclopropyl-N-(3-(4-(3-methylbenzylamino)quinazolin-6-yl)benzyl)cyclohexane carboxamide,
469 N-(5-(4-(benzylamino)quinazolin-6-yl)-2-fluorobenzyl)-N-methylquinoline-8-sulfonamide,
470 N-cyclopropyl-N-(5-(4-(cyclopropylamino)-2-methylquinazolin-6-yl)-2-fluorobenzyl)-2-(trifluoromethyl)benzamide,
471 N-cyclopropyl-N-(3-(4-(cyclopropylamino)-2-methylquinazolin-6-yl)benzyl)quinoline-8-sulfonamide,
472 2,4-dichloro-N-cyclopropyl-N-(3-(4-(phenethylamino)quinazolin-6-yl)benzyl)benzole sulfonamide,
473 N-cyclopropyl-N-(3-(4-(3-methylbenzylamino)quinazolin-6-yl)benzyl)-3-(trifluoromethyl)benzole sulfonamide,
474 1,3-dimethyl-N-(3-(4-(methylamino)quinazolin-6-yl)benzyl)-N-(pyridin-2-ylmethyl)-1H-pyrazole-5-carboxamide,
475 N-(3-(4-(methylamino)quinazolin-6-yl)benzyl)-N-(pyridin-2-ylmethyl)nicotinamide,
476 N-(5-(4-(benzylamino)quinazolin-6-yl)-2-fluorobenzyl)-N-methylmethane sulfonamide,
477 N-cyclopropyl-N-(3-(4-(cyclopropylamino)-2-methylquinazolin-6-yl)benzyl)isoxazole-5-carboxamide,
478 N-cyclopropyl-N-(3-(4-(cyclopropylamino)-2-methylquinazolin-6-yl)benzyl)methane sulfonamide,
479 3,5-dichloro-N-cyclopropyl-N-(3-(4-(cyclopropylamino)-2-methylquinazolin-6-yl)benzyl)benzamide,
480 N-cyclopropyl-2,3,4,5,6-pentafluoro-N-(3-(4-(phenethylamino)quinazolin-6-yl)benzyl)benzole sulfonamide,
481 N-(3-(4-(methylamino)quinazolin-6-yl)benzyl)-N-(pyridin-2-ylmethyl)methane sulfonamide,
482 N-(3-(4-(methylamino)quinazolin-6-yl)benzyl)-N-(pyridin-2-ylmethyl)benzamide,
483 N-(3-(4-(methylamino)quinazolin-6-yl)benzyl)-N-(pyridin-2-ylmethyl)acetamide,
484 N-(5-(4-(benzylamino)quinazolin-6-yl)-2-fluorobenzyl)-N-methylpropane-2-sulfonamide,
485 N-cyclopropyl-N-(3-(4-(cyclopropylamino)-2-methylquinazolin-6-yl)benzyl)-1,3-dimethyl-1H-pyrazole-5-carboxamide,
486 N-cyclopropyl-N-(3-(4-(cyclopropylamino)-2-methylquinazolin-6-yl)benzyl)propane-2-sulfonamide,
487 N-cyclopropyl-N-(3-(4-(cyclopropylamino)-2-methylquinazolin-6-yl)benzyl)picolinamide,
488 N-cyclopropyl-N-(3-(4-(phenethylamino)quinazolin-6-yl)benzyl)thiophene-2-sulfonamide,
489 N-(3-(4-(methylamino)quinazolin-6-yl)benzyl)-1-phenyl-N-(pyridin-2-ylmethyl)methane sulfonamide,
490 3,4-dimethoxy-N-(3-(4-(methylamino)quinazolin-6-yl)benzyl)-N-(pyridin-2-ylmethyl)benzamide,
491 N-(3-(4-(methylamino)quinazolin-6-yl)benzyl)-N-(pyridin-2-ylmethyl)pentanamide,
492 N-(5-(4-(benzylamino)quinazolin-6-yl)-2-fluorobenzyl)-N-methyl-1-phenylmethane sulfonamide,
493 N-cyclopropyl-N-(3-(4-(cyclopropylamino)-2-methylquinazolin-6-yl)benzyl)benzamide,
494 N-cyclopropyl-N-(3-(4-(cyclopropylamino)-2-methylquinazolin-6-yl)benzyl)-1-phenylmethane sulfonamide,
495 N-cyclopropyl-N-(3-(4-(cyclopropylamino)-2-methylquinazolin-6-yl)benzyl)isonicotinamide,
496 N-cyclopropyl-N-(3-(4-(phenethylamino)quinazolin-6-yl)benzyl)quinoline-8-sulfonamide,
497 N-(3-(4-(methylamino)quinazolin-6-yl)benzyl)-N-(pyridin-2-ylmethyl)benzole sulfonamide,
498 3-fluoro-N-(3-(4-(methylamino)quinazolin-6-yl)benzyl)-N-(pyridin-2-ylmethyl)benzamide,
499 2-methoxy-N-(3-(4-(methylamino)quinazolin-6-yl)benzyl)-N-(pyridin-2-ylmethyl)acetamide,
500 N-(5-(4-(benzylamino)quinazolin-6-yl)-2-fluorobenzyl)-N-methylbenzole sulfonamide,
501 N-cyclopropyl-N-(3-(4-(cyclopropylamino)-2-methylquinazolin-6-yl)benzyl)-3,4-dimethoxybenzamide,
502 N-cyclopropyl-N-(3-(4-(cyclopropylamino)-2-methylquinazolin-6-yl)benzyl)benzole sulfonamide,
503 N-cyclopropyl-N-(3-(4-(cyclopropylamino)-2-methylquinazolin-6-yl)benzyl)nicotinamide,
504 N-cyclopropyl-N-(3-(4-(3-methylbenzylamino)quinazolin-6-yl)benzyl)propane-2-sulfonamide,
505 N-(3-(4-(methylamino)quinazolin-6-yl)benzyl)-N-(pyridin-2-ylmethyl)-3-(trifluoromethyl)benzole sulfonamide,
506 2,4-difluoro-N-(3-(4-(methylamino)quinazolin-6-yl)benzyl)-N-(pyridin-2-ylmethyl)benzamide,
507 N-(3-(4-(methylamino)quinazolin-6-yl)benzyl)-N-(pyridin-2-ylmethyl)cyclohexane carboxamide,
508 N-(5-(4-(benzylamino)quinazolin-6-yl)-2-fluorobenzyl)-N-methyl-3-(trifluoromethyl)benzole sulfonamide,
509 N-cyclopropyl-N-(3-(4-(cyclopropylamino)-2-methylquinazolin-6-yl)benzyl)-3-fluorobenzamide,
510 N-cyclopropyl-N-(3-(4-(cyclopropylamino)-2-methylquinazolin-6-yl)benzyl)-3-(trifluoromethyl)benzole sulfonamide,
511 N-cyclopropyl-N-(3-(4-(cyclopropylamino)-2-methylquinazolin-6-yl)benzyl)acetamide,
512 N-cyclopropyl-N-(3-(4-(3-methylbenzylamino)quinazolin-6-yl)benzyl)isoxazole-5-carboxamide,
513 4-methoxy-N-(3-(4-(methylamino)quinazolin-6-yl)benzyl)-N-(pyridin-2-ylmethyl)benzole sulfonamide,
514 2-fluoro-N-(3-(4-(methylamino)quinazolin-6-yl)benzyl)-N-(pyridin-2-ylmethyl)-4-(trifluoromethyl)benzamide,
515 N-(3-(4-(methylamino)quinazolin-6-yl)benzyl)-2-phenyl-N-(pyridin-2-ylmethyl)acetamide,
516 N-(5-(4-(benzylamino)quinazolin-6-yl)-2-fluorobenzyl)-4-methoxy-N-methylbenzole sulfonamide,
517 N-cyclopropyl-N-(3-(4-(cyclopropylamino)-2-methylquinazolin-6-yl)benzyl)-2,4-difluorobenzamide,
518 N-cyclopropyl-N-(3-(4-(cyclopropylamino)-2-methylquinazolin-6-yl)benzyl)-4-methoxybenzole sulfonamide,
519 N-cyclopropyl-N-(3-(4-(cyclopropylamino)-2-methylquinazolin-6-yl)benzyl)pentanamide,
520 N-cyclopropyl-N-(3-(4-(cyclopropylamino)-2-methylquinazoline-6-yl)benzyl)-2-methoxyacetamide,
521 N-(2-fluoro-5-(4-(methylamino)quinazolin-6-yl)benzyl)-N-methylfuran-2-carboxamide,
522 N-(2-fluoro-5-(4-(methylamino)quinazolin-6-yl)benzyl)-N-methyl-3-(trifluoromethyl)benzamide,
523 N-(2-fluoro-5-(4-(methylamino)quinazolin-6-yl)benzyl)-N-methyl-3-(trifluoromethyl)benzole sulfonamide,
524 N-cyclopropyl-N-(5-(4-(cyclopropylamino)-2-methylquinazolin-6-yl)-2-fluorobenzyl)-2,4-difluorobenzamide,
525 N-(5-(4-(benzylamino)quinazolin-6-yl)-2-fluorobenzyl)-N-methyl-2-(trifluoromethyl)benzamide,
526 N-cyclopropyl-N-(5-(4-(cyclopropylamino)-2-methylquinazolin-6-yl)-2-fluorobenzyl)furan-2-carboxamide,
527 N-cyclopropyl-N-(5-(4-(cyclopropylamino)-2-methylquinazolin-6-yl)-2-fluorobenzyl)methane sulfonamide,
528 N-cyclopropyl-N-(3-(4-(cyclopropylamino)-2-methylquinazolin-6-yl)benzyl)-2-(dimethylamino)acetamide,
529 N-(2-fluoro-5-(4-(methylamino)quinazolin-6-yl)benzyl)-N-methylthiophene-2-carboxamide,
530 N-(2-fluoro-5-(4-(methylamino)quinazolin-6-yl)benzyl)-N-methyl-2-(trifluoromethyl)benzamide,
531 2,4-dichloro-N-(2-fluoro-5-(4-(methylamino)quinazolin-6-yl)benzyl)-N-methylbenzole sulfonamide,
532 N-cyclopropyl-N-(5-(4-(cyclopropylamino)-2-methylquinazolin-6-yl)-2-fluorobenzyl)-2-fluoro-4-(trifluoromethyl)benzamide,
533 N-(5-(4-(benzylamino)quinazolin-6-yl)-2-fluorobenzyl)-3,5-dichloro-N-methylbenzamide,
534 N-cyclopropyl-N-(5-(4-(cyclopropylamino)-2-methylquinazolin-6-yl)-2-fluorobenzyl)thiophene-2-carboxamide,
535 N-cyclopropyl-N-(5-(4-(cyclopropylamino)-2-methylquinazolin-6-yl)-2-fluorobenzyl)propane-2-sulfonamide,
536 N-cyclopropyl-N-(3-(4-(cyclopropylamino)-2-methylquinazolin-6-yl)benzyl)cyclohexane carboxamide,
537 N-(2-fluoro-5-(4-(methylamino)quinazolin-6-yl)benzyl)-N-methylisoxazole-5-carboxamide,
538 N-(2-fluoro-5-(4-(methylamino)quinazolin-6-yl)benzyl)-N-methylacetamide,
539 2,3,4,5,6-pentafluoro-N-(2-fluoro-5-(4-(methylamino)quinazolin-6-yl)benzyl)-N-methylbenzole sulfonamide,
540 N-cyclopropyl-N-(5-(4-(cyclopropylamino)-2-methylquinazolin-6-yl)-2-fluorobenzyl)pentanamide,
541 N-(5-(4-(benzylamino)quinazolin-6-yl)-2-fluorobenzyl)-N-methyl pivalamide,
542 N-cyclopropyl-N-(5-(4-(cyclopropylamino)-2-methylquinazolin-6-yl)-2-fluorobenzyl)-1,3-dimethyl-1H-pyrazole-5-carboxamide,
543 N-cyclopropyl-N-(5-(4-(cyclopropylamino)-2-methylquinazolin-6-yl)-2-fluorobenzyl)-1-phenylmethane sulfonamide,
544 N-cyclopropyl-N-(3-(4-(cyclopropylamino)-2-methylquinazolin-6-yl)benzyl)-2-phenylacetamide,
545 N-(2-fluoro-5-(4-(methylamino)quinazolin-6-yl)benzyl)-N,1,3-trimethyl-1H-pyrazole-5-carboxamide,
546 N-(2-fluoro-5-(4-(methylamino)quinazolin-6-yl)benzyl)-N-methyl cyclohexane carboxamide,
547 N-(2-fluoro-5-(4-(methylamino)quinazolin-6-yl)benzyl)-N-methylthiophene-2-sulfonamide,
548 N-(5-(4-(benzylamino)quinazolin-6-yl)-2-fluorobenzyl)-N,1,3-trimethyl-1H-pyrazole-5-carboxamide,
549 N-(5-(4-(benzylamino)quinazolin-6-yl)-2-fluorobenzyl)-N-methyl pentanamide,
550 N-cyclopropyl-N-(5-(4-(cyclopropylamino)-2-methylquinazolin-6-yl)-2-fluorobenzyl)benzamide,
551 N-cyclopropyl-N-(5-(4-(cyclopropylamino)-2-methylquinazolin-6-yl)-2-fluorobenzyl)benzole sulfonamide,
552 N-cyclopropyl-N-(3-(4-(cyclopropylamino)-2-methylquinazolin-6-yl)benzyl)-3-phenylpropanamide,
553 N-(2-fluoro-5-(4-(methylamino)quinazolin-6-yl)benzyl)-N-methylbenzamide,
554 N-(2-fluoro-5-(4-(methylamino)quinazolin-6-yl)benzyl)-2-methoxy-N-methylacetamide,
555 N-(2-fluoro-5-(4-(methylamino)quinazolin-6-yl)benzyl)-N-methylquinoline-8-sulfonamide,
556 N-(5-(4-(benzylamino)quinazolin-6-yl)-2-fluorobenzyl)-N-methylbenzamide,
557 N-(5-(4-(benzylamino)quinazolin-6-yl)-2-fluorobenzyl)-2-methoxy-N-methylacetamide,
558 N-cyclopropyl-N-(5-(4-(cyclopropylamino)-2-methylquinazolin-6-yl)-2-fluorobenzyl)-3,4-dimethoxybenzamide,
559 N-cyclopropyl-N-(5-(4-(cyclopropylamino)-2-methylquinazolin-6-yl)-2-fluorobenzyl)-3-(trifluoromethyl)benzole sulfonamide,
560 N-(2-fluoro-5-(4-(methylamino)quinazolin-6-yl)benzyl)-4-methoxy-N-methylbenzole sulfonamide,
561 N-(2-fluoro-5-(4-(methylamino)quinazolin-6-yl)benzyl)-3,4-dimethoxy-N-methylbenzamide,
562 N-(2-fluoro-5-(4-(methylamino)quinazolin-6-yl)benzyl)-N-methyl-3-phenylpropanamide,
563 N-(5-(4-(benzylamino)quinazolin-6-yl)-2-fluorobenzyl)-N-methylthiophene-2-carboxamide,
564 N-(5-(4-(benzylamino)quinazolin-6-yl)-2-fluorobenzyl)-3,4-dimethoxy-N-methylbenzamide,
565 N-(5-(4-(benzylamino)quinazolin-6-yl)-2-fluorobenzyl)-2-(dimethylamino)-N-methylacetamide,
566 N-cyclopropyl-N-(5-(4-(cyclopropylamino)-2-methylquinazolin-6-yl)-2-fluorobenzyl)-3-fluorobenzamide,
567 N-cyclopropyl-N-(5-(4-(cyclopropylamino)-2-methylquinazolin-6-yl)-2-fluorobenzyl)-4-methoxybenzole sulfonamide,
568 N-(2-fluoro-5-(4-(methylamino)quinazolin-6-yl)benzyl)-N-methylisonicotinamide,
569 3-fluoro-N-(2-fluoro-5-(4-(methylamino)quinazolin-6-yl)benzyl)-N-methylbenzamide,
570 N-(2-fluoro-5-(4-(methylamino)quinazolin-6-yl)benzyl)-N-methylmethane sulfonamide,
571 N-(5-(4-(benzylamino)quinazolin-6-yl)-2-fluorobenzyl)-3-fluoro-N-methylbenzamide,
572 N-(5-(4-(benzylamino)quinazolin-6-yl)-2-fluorobenzyl)-2,4-difluoro-N-methylbenzamide,
573 N-(2-fluoro-5-(4-(methylamino)quinazolin-6-yl)benzyl)-N-methyl nicotinamide,
574 N-cyclopropyl-N-(5-(4-(cyclopropylamino)-2-methylquinazolin-6-yl)-2-fluorobenzyl)-2,3,4,5,6-pentafluorobenzamide,
575 2,4-dichloro-N-cyclopropyl-N-(5-(4-(cyclopropylamino)-2-methylquinazolin-6-yl)-2-fluorobenzyl)benzole sulfonamide
576 N-(2-fluoro-5-(4-(methylamino)quinazolin-6-yl)benzyl)-N-methylpentanamide,
577 2,4-difluoro-N-(2-fluoro-5-(4-(methylamino)quinazolin-6-yl)benzyl)-N-methylbenzamide,
578 N-(2-fluoro-5-(4-(methylamino)quinazolin-6-yl)benzyl)-N-methylpropane-2-sulfonamide,
579 N-(5-(4-(benzylamino)quinazolin-6-yl)-2-fluorobenzyl)-N-methyl isonicotinamide,
580 N-(5-(4-(benzylamino)quinazolin-6-yl)-2-fluorobenzyl)-2,3,4,5,6-pentafluoro-N-methylbenzamide,
581 N-(5-(4-(benzylamino)quinazolin-6-yl)-2-fluorobenzyl)-N-methyl cyclohexane carboxamide,
582 N-cyclopropyl-N-(5-(4-(cyclopropylamino)-2-methylquinazolin-6-yl)-2-fluorobenzyl)-3-(trifluoromethyl)benzamide,
583 N-cyclopropyl-N-(5-(4-(cyclopropylamino)-2-methylquinazolin-6-yl)-2-fluorobenzyl)-2,3,4,5,6-pentafluorobenzole sulfonamide,
584 N-(2-fluoro-5-(4-(methylamino)quinazolin-6-yl)benzyl)-N-methyl-2-phenylacetamide,
585 2,3,4,5,6-pentafluoro-N-(2-fluoro-5-(4-(methylamino)quinazolin-6-yl)benzyl)-N-methylbenzamide,
586 N-(2-fluoro-5-(4-(methylamino)quinazolin-6-yl)benzyl)-N-methyl-1-phenylmethane sulfonamide,
587 N-(5-(4-(benzylamino)quinazolin-6-yl)-2-fluorobenzyl)-N-methyl nicotinamide,
588 N-(5-(4-(benzylamino)quinazolin-6-yl)-2-fluorobenzyl)-2-fluoro-N-methyl-4-(trifluoromethyl)benzamide,
589 N-(5-(4-(benzylamino)quinazolin-6-yl)-2-fluorobenzyl)-N-methyl-2-phenylacetamide,
590 N-(2-fluoro-5-(4-(methylamino)quinazolin-6-yl)benzyl)-N-methyl pivalamide,
591 N-cyclopropyl-N-(5-(4-(cyclopropylamino)-2-methylquinazolin-6-yl)-2-fluorobenzyl)thiophene-2-sulfonamide,
592 N-(2-fluoro-5-(4-(methylamino)quinazolin-6-yl)benzyl)-N-methyl picolinamide,
593 2-fluoro-N-(2-fluoro-5-(4-(methylamino)quinazolin-6-yl)benzyl)-N-methyl-4-(trifluoromethyl)benzamide,
594 N-(2-fluoro-5-(4-(methylamino)quinazolin-6-yl)benzyl)-N-methyl benzole sulfonamide,
595 N-(5-(4-(benzylamino)quinazolin-6-yl)-2-fluorobenzyl)-N-methylacetamide,
596 N-(5-(4-(benzylamino)quinazolin-6-yl)-2-fluorobenzyl)-N-methyl-3-(trifluoromethyl)benzamide,
597 N-(5-(4-(benzylamino)quinazolin-6-yl)-2-fluorobenzyl)-N-methyl-3-phenylpropanamide,
598 N-(5-(4-(benzylamino)quinazolin-6-yl)-2-fluorobenzyl)-N-methylfuran-2-carboxamide,
599 N-cyclopropyl-N-(5-(4-(cyclopropylamino)-2-methylquinazolin-6-yl)-2-fluorobenzyl)quinoline-8-sulfonamide,
600 3,5-dichloro-N-cyclopropyl-N-(5-(4-(cyclopropylamino)-2-methylquinazolin-6-yl)-2-fluorobenzyl)benzamide,
601 N-cyclopropyl-N-(5-(4-(cyclopropylamino)-2-methylquinazolin-6-yl)-2-fluorobenzyl)-3-phenylpropanamide,
602 N-cyclopropyl-N-(3-(4-(2,4-dimethoxybenzylamino)quinazolin-6-yl)benzyl)benzole sulfonamide,
603 N-cyclopropyl-N-(3-(4-(2,4-dimethoxybenzylamino)quinazolin-6-yl)benzyl)isonicotinamide,
604 N-cyclopropyl-N-(5-(4-(cyclopropylamino)-2-methylquinazolin-6-yl)-2-fluorobenzyl)picolinamide,
605 N-cyclopropyl-N-(5-(4-(cyclopropylamino)quinazolin-6-yl)-2-fluorobenzyl)-2-methylthiazole-4-carboxamide,
606 N-cyclopropyl-N-(3-(4-(2,4-dimethoxybenzylamino)quinazolin-6-yl)benzyl)-3-(trifluoromethyl)benzole sulfonamide,
607 N-cyclopropyl-N-(3-(4-(2,4-dimethoxybenzylamino)quinazolin-6-yl)benzyl)benzamide,
608 N-cyclopropyl-N-(5-(4-(cyclopropylamino)-2-methylquinazolin-6-yl)-2-fluorobenzyl)isonicotinamide,
609 N-cyclopropyl-2-methyl-N-(3-(4-(phenethylamino)quinazolin-6-yl)benzyl)thiazole-4-carboxamide,
610 N-cyclopropyl-N-(3-(4-(2,4-dimethoxybenzylamino)quinazolin-6-yl)benzyl)-4-methoxybenzole sulfonamide,
611 N-cyclopropyl-N-(3-(4-(2,4-dimethoxybenzylamino)quinazolin-6-yl)benzyl)-3-fluorobenzamide,
612 N-cyclopropyl-N-(5-(4-(cyclopropylamino)-2-methylquinazolin-6-yl)-2-fluorobenzyl)nicotinamide,
613 N-cyclopropyl-N-(3-(4-(2,4-dimethoxybenzylamino)quinazolin-6-yl)benzyl)-2-methylthiazole-4-carboxamide,
614 2,4-dichloro-N-cyclopropyl-N-(3-(4-(2,4-dimethoxybenzylamino)quinazolin-6-yl)benzyl)benzole sulfonamide,
615 N-cyclopropyl-N-(3-(4-(2,4-dimethoxybenzylamino)quinazolin-6-yl)benzyl)-2,4-difluorobenzamide,
616 N-cyclopropyl-N-(5-(4-(cyclopropylamino)-2-methylquinazolin-6-yl)-2-fluorobenzyl)acetamide,
617 N-(2-fluoro-5-(4-(methylamino)quinazolin-6-yl)benzyl)-N,2-dimethylthiazole-4-carboxamide,
618 N-cyclopropyl-2-methyl-N-(3-(4-(3-methylbenzylamino)quinazolin-6-yl)benzyl)thiazole-4-carboxamide,
619 N-cyclopropyl-N-(3-(4-(cyclopropylamino)-8-fluoroquinazolin-6-yl)benzyl)-2,4-difluorobenzamide,
620 N-cyclopropyl-N-(3-(4-(2,4-dimethoxybenzylamino)quinazolin-6-yl)benzyl)-2,3,4,5,6-pentafluorobenzamide,
621 N-cyclopropyl-N-(5-(4-(cyclopropylamino)-2-methylquinazolin-6-yl)-2-fluorobenzyl)pivalamide,
622 N-(5-(4-(benzylamino)quinazolin-6-yl)-2-fluorobenzyl)-N,2-dimethylthiazole-4-carboxamide,
623 2,4-dichloro-N-(3-(4-(methylamino)quinazolin-6-yl)benzyl)-N-(pyridin-2-ylmethyl)benzole sulfonamide,
624 N-cyclopropyl-N-(5-(4-(cyclopropylamino)-2-methylquinazolin-6-yl)-2-fluorobenzyl)-2-methylthiazole-4-carboxamide,
625 N-cyclopropyl-N-(3-(4-(2,4-dimethoxybenzylamino)quinazolin-6-yl)benzyl)thiophene-2-sulfonamide,
626 N-cyclopropyl-N-(3-(4-(2,4-dimethoxybenzylamino)quinazolin-6-yl)benzyl)-2-fluoro-4-(trifluoromethyl)benzamide,
627 N-cyclopropyl-N-(5-(4-(cyclopropylamino)-2-methylquinazolin-6-yl)-2-fluorobenzyl)-2-methoxyacetamide,
628 N-(3-(4-(methylamino)quinazolin-6-yl)benzyl)-N-(pyridin-2-ylmethyl)pivalamide,
629 N-(3-(4-(methylamino)quinazolin-6-yl)benzyl)-N-(pyridin-2-ylmethyl)thiophene-2-sulfonamide,
630 2-methyl-N-(3-(4-(methylamino)quinazolin-6-yl)benzyl)-N-(pyridin-2-ylmethyl)thiazole-4-carboxamide,
631 N-cyclopropyl-N-(3-(4-(2,4-dimethoxybenzylamino)quinazolin-6-yl)benzyl)quinoline-8-sulfonamide,
632 N-cyclopropyl-N-(3-(4-(2,4-dimethoxybenzylamino)quinazolin-6-yl)benzyl)-3-(trifluoromethyl)benzamide,
633 N-cyclopropyl-N-(5-(4-(cyclopropylamino)-2-methylquinazolin-6-yl)-2-fluorobenzyl)-2-(dimethylamino)acetamide,
634 N-cyclopropyl-N-(3-(4-(cyclopropylamino)-2-methylquinazolin-6-yl)benzyl)-2-methylthiazole-4-carboxamide,
635 N-(3-(4-(methylamino)quinazolin-6-yl)benzyl)-N-(pyridin-2-ylmethyl)propane-2-sulfonamide,
636 N-(5-(4-(benzylamino)quinazolin-6-yl)-2-fluorobenzyl)-N-methyl picolinamide,
637 N-cyclopropyl-N-(3-(4-(2,4-dimethoxybenzylamino)quinazolin-6-yl)benzyl)furan-2-carboxamide,
638 N-cyclopropyl-N-(3-(4-(2,4-dimethoxybenzylamino)quinazolin-6-yl)benzyl)-2-(trifluoromethyl)benzamide,
639 N-cyclopropyl-N-(5-(4-(cyclopropylamino)-2-methylquinazolin-6-yl)-2-fluorobenzyl)cyclohexane carboxamide,
640 N-cyclopropyl-N-(3-(4-(cyclopropylamino)-2-methylquinazolin-6-yl)benzyl)pivalamide,
641 N-cyclopropyl-N-(3-(4-(2,4-dimethoxybenzylamino)quinazolin-6-yl)benzyl)methane sulfonamide,
642 N-cyclopropyl-N-(3-(4-(2,4-dimethoxybenzylamino)quinazolin-6-yl)benzyl)thiophene-2-carboxamide,
643 3,5-dichloro-N-cyclopropyl-N-(3-(4-(2,4-dimethoxybenzylamino)quinazolin-6-yl)benzyl)benzamide,
644 N-cyclopropyl-N-(5-(4-(cyclopropylamino)-2-methylquinazolin-6-yl)-2-fluorobenzyl)-2-phenylacetamide,
645 N-cyclopropyl-N-(5-(4-(cyclopropylamino)quinazolin-6-yl)-2-fluorobenzyl)pivalamide,
646 N-cyclopropyl-N-(3-(4-(2,4-dimethoxybenzylamino)quinazolin-6-yl)benzyl)-1-phenylmethane sulfonamide,
647 N-cyclopropyl-N-(3-(4-(2,4-dimethoxybenzylamino)quinazolin-6-yl)benzyl)isoxazole-5-carboxamide,
648 N-cyclopropyl-N-(3-(4-(2,4-dimethoxybenzylamino)quinazolin-6-yl)benzyl)picolinamide,
649 N-cyclopropyl-N-(3-(4-(2,4-dimethoxybenzylamino)quinazolin-6-yl)benzyl)nicotinamide,
650 N-cyclopropyl-N-(3-(4-(cyclopropylamino)-2-methylquinazolin-6-yl)benzyl)furan-2-carboxamide,
651 N-cyclopropyl-N-(3-(4-(cyclopropylamino)-7-methylquinazolin-6-yl)benzyl)-3-(trifluoromethyl)benzole sulfonamide,
652 N-cyclopropyl-N-(3-(4-(cyclopropylamino)-7-methylquinazolin-6-yl)benzyl)-3-fluorobenzamide,
653 N-cyclopropyl-N-(3-(4-(cyclopropylamino)-7-methylquinazolin-6-yl)benzyl)cyclohexane carboxamide,
654 N-cyclopropyl-N-(3-(4-(cyclopropylamino)-8-fluoroquinazolin-6-yl)benzyl)pentanamide,
655 N-(2-fluorophenyl)-N-(3-(4-(methylamino)quinazolin-6-yl)benzyl)thiophene-2-sulfonamide,
656 N-cyclopropyl-N-(3-(4-(2,4-dimethoxybenzylamino)quinazolin-6-yl)benzyl)-3,4-dimethoxybenzamide,
657 N-cyclopropyl-N-(3-(4-(2,4-dimethoxybenzylamino)quinazolin-6-yl)benzyl)acetamide,
658 N-cyclopropyl-N-(3-(4-(cyclopropylamino)-2-methylquinazolin-6-yl)benzyl)thiophene-2-carboxamide,
659 N-cyclopropyl-N-(3-(4-(cyclopropylamino)-7-methylquinazolin-6-yl)benzyl)-4-methoxybenzole sulfonamide,
660 N-cyclopropyl-N-(3-(4-(cyclopropylamino)-7-methylquinazolin-6-yl)benzyl)-2,4-difluorobenzamide,
661 N-cyclopropyl-N-(3-(4-(cyclopropylamino)-7-methylquinazolin-6-yl)benzyl)-2-phenylacetamide,
662 N-cyclopropyl-N-(3-(4-(cyclopropylamino)-8-fluoroquinazolin-6-yl)benzyl)cyclohexane carboxamide,
663 N-(2-fluorophenyl)-N-(3-(4-(methylamino)quinazolin-6-yl)benzyl)quinoline-8-sulfonamide,
664 N-(5-(4-(benzylamino)quinazolin-6-yl)-2-fluorobenzyl)-N-methylisoxazole-5-carboxamide,
665 N-cyclopropyl-N-(3-(4-(2,4-dimethoxybenzylamino)quinazolin-6-yl)benzyl)pivalamide,
666 N-cyclopropyl-N-(3-(4-(cyclopropylamino)-2-methylquinazolin-6-yl)benzyl)-2-(trifluoromethyl)benzamide,
667 2,4-dichloro-N-cyclopropyl-N-(3-(4-(cyclopropylamino)-7-methylquinazolin-6-yl)benzyl)benzole sulfonamide,
668 N-cyclopropyl-N-(3-(4-(cyclopropylamino)-7-methylquinazolin-6-yl)benzyl)-2-fluoro-4-(trifluoromethyl)benzamide,
669 N-cyclopropyl-N-(3-(4-(cyclopropylamino)-7-methylquinazolin-6-yl)benzyl)-3-phenylpropanamide,
670 N-cyclopropyl-N-(3-(4-(cyclopropylamino)-8-fluoroquinazolin-6-yl)benzyl)-2-phenylacetamide,
671 N-(2-fluorophenyl)-N-(3-(4-(methylamino)quinazolin-6-yl)benzyl)isoxazole-5-carboxamide,
672 N-(2-fluorophenyl)-N-(3-(4-(methylamino)quinazolin-6-yl)benzyl)-3-(trifluoromethyl)benzole sulfonamide,
673 N-cyclopropyl-N-(3-(4-(2,4-dimethoxybenzylamino)quinazolin-6-yl)benzyl)pentanamide,
674 3,5-dichloro-N-(2-fluoro-5-(4-(methylamino)quinazolin-6-yl)benzyl)-N-methylbenzamide,
675 N-cyclopropyl-N-(3-(4-(cyclopropylamino)-7-methylquinazolin-6-yl)benzyl)-2,3,4,5,6-pentafluorobenzole sulfonamide,
676 N-cyclopropyl-N-(3-(4-(cyclopropylamino)-7-methylquinazolin-6-yl)benzyl)-3-(trifluoromethyl)benzamide,
677 N-cyclopropyl-N-(3-(4-(cyclopropylamino)-8-fluoroquinazolin-6-yl)benzyl)furan-2-carboxamide,
678 N-cyclopropyl-N-(3-(4-(cyclopropylamino)-8-fluoroquinazolin-6-yl)benzyl)methane sulfonamide,
679 N-(2-fluorophenyl)-N-(3-(4-(methylamino)quinazolin-6-yl)benzyl)benzamide,
680 N-(2-fluorophenyl)-4-methoxy-N-(3-(4-(methylamino)quinazolin-6-yl)benzyl)benzole sulfonamide,
681 N-cyclopropyl-N-(3-(4-(2,4-dimethoxybenzylamino)quinazolin-6-yl)benzyl)-2-methoxyacetamide,
682 N-cyclopropyl-N-(5-(4-(cyclopropylamino)-2-methylquinazolin-6-yl)-2-fluorobenzyl)isoxazole-5-carboxamide,
683 N-cyclopropyl-N-(3-(4-(cyclopropylamino)-7-methylquinazolin-6-yl)benzyl)quinoline-8-sulfonamide,
684 N-cyclopropyl-N-(3-(4-(cyclopropylamino)-7-methylquinazolin-6-yl)benzyl)-2-(trifluoromethyl)benzamide,
685 N-cyclopropyl-N-(3-(4-(cyclopropylamino)-8-fluoroquinazolin-6-yl)benzyl)thiophene-2-carboxamide,
686 N-cyclopropyl-N-(3-(4-(cyclopropylamino)-8-fluoroquinazolin-6-yl)benzyl)-3-(trifluoromethyl)benzole sulfonamide,
687 3-fluoro-N-(2-fluorophenyl)-N-(3-(4-(methylamino)quinazolin-6-yl)benzyl)benzamide,
688 N-(3-(4-(methylamino)quinazolin-6-yl)benzyl)-N-(pyridin-2-ylmethyl)isoxazole-5-carboxamide,
689 N-cyclopropyl-N-(3-(4-(2,4-dimethoxybenzylamino)quinazolin-6-yl)benzyl)-2-(dimethylamino)acetamide,
690 N-cyclopropyl-N-(3-(4-(cyclopropylamino)-8-fluoroquinazolin-6-yl)benzyl)benzole sulfonamide,
691 N-cyclopropyl-N-(3-(4-(cyclopropylamino)-7-methylquinazolin-6-yl)benzyl)furan-2-carboxamide,
692 3,5-dichloro-N-cyclopropyl-N-(3-(4-(cyclopropylamino)-7-methylquinazolin-6-yl)benzyl)benzamide,
693 N-cyclopropyl-N-(3-(4-(cyclopropylamino)-8-fluoroquinazolin-6-yl)benzyl)benzamide,
694 N-cyclopropyl-N-(3-(4-(cyclopropylamino)-8-fluoroquinazolin-6-yl)benzyl)-4-methoxybenzole sulfonamide,
695 2,3,4,5,6-pentafluoro-N-(2-fluorophenyl)-N-(3-(4-(methylamino)quinazolin-6-yl)benzyl)benzamide,
696 N-cyclopropyl-N-(3-(4-(2,4-dimethoxybenzylamino)quinazolin-6-yl)benzyl)cyclohexane carboxamide,
697 N-cyclopropyl-N-(3-(4-(cyclopropylamino)-7-methylquinazolin-6-yl)benzyl)methane sulfonamide,
698 N-cyclopropyl-N-(3-(4-(cyclopropylamino)-7-methylquinazolin-6-yl)benzyl)thiophene-2-carboxamide,
699 N-cyclopropyl-N-(3-(4-(cyclopropylamino)-7-methylquinazolin-6-yl)benzyl)picolinamide,
700 N-cyclopropyl-N-(3-(4-(cyclopropylamino)-8-fluoroquinazolin-6-yl)benzyl)-3,4-dimethoxybenzamide,
701 N-cyclopropyl-N-(3-(4-(cyclopropylamino)-8-fluoroquinazolin-6-yl)benzyl)thiophene-2-sulfonamide,
702 N-cyclopropyl-N-(3-(4-(cyclopropylamino)-7-methylquinazolin-6-yl)benzyl)nicotinamide,
703 N-cyclopropyl-N-(3-(4-(cyclopropylamino)-8-fluoroquinazolin-6-yl)benzyl)-3-phenylpropanamide,
704 N-cyclopropyl-N-(3-(4-(2,4-dimethoxybenzylamino)quinazolin-6-yl)benzyl)-2-phenylacetamide,
705 N-cyclopropyl-N-(3-(4-(cyclopropylamino)-7-methylquinazolin-6-yl)benzyl)propane-2-sulfonamide,
706 N-cyclopropyl-N-(3-(4-(cyclopropylamino)-7-methylquinazolin-6-yl)benzyl)-1,3-dimethyl-1H-pyrazole-5-carboxamide,
707 N-cyclopropyl-N-(3-(4-(cyclopropylamino)-7-methylquinazolin-6-yl)benzyl)isonicotinamide,
708 N-cyclopropyl-N-(3-(4-(cyclopropylamino)-8-fluoroquinazolin-6-yl)benzyl)picolinamide,
709 N-cyclopropyl-N-(3-(4-(cyclopropylamino)-8-fluoroquinazolin-6-yl)benzyl)quinoline-8-sulfonamide,
710 N-cyclopropyl-N-(3-(4-(cyclopropylamino)-7-methylquinazolin-6-yl)benzyl)acetamide,
711 N-(2-fluorophenyl)-N-(3-(4-(methylamino)quinazolin-6-yl)benzyl)acetamide,
712 N-cyclopropyl-N-(3-(4-(2,4-dimethoxybenzylamino)quinazolin-6-yl)benzyl)-3-phenylpropanamide,
713 N-cyclopropyl-N-(3-(4-(cyclopropylamino)-7-methylquinazolin-6-yl)benzyl)-1-phenylmethane sulfonamide,
714 N-cyclopropyl-N-(3-(4-(cyclopropylamino)-7-methylquinazolin-6-yl)benzyl)benzamide,
715 N-cyclopropyl-N-(3-(4-(cyclopropylamino)-7-methylquinazolin-6-yl)benzyl)pentanamide,
716 N-cyclopropyl-N-(3-(4-(cyclopropylamino)-8-fluoroquinazolin-6-yl)benzyl)acetamide,
717 N-(2-fluorophenyl)-N-(3-(4-(methylamino)quinazolin-6-yl)benzyl)methane sulfonamide,
718 2-(dimethylamino)-N-(2-fluoro-5-(4-(methylamino)quinazolin-6-yl)benzyl)-N-methylacetamide,
719 N-cyclopropyl-N-(3-(4-(cyclopropylamino)-7-methylquinazolin-6-yl)benzyl)pivalamide,
720 N-(3-(4-(methylamino)quinazolin-6-yl)benzyl)-N-(pyridin-2-ylmethyl)-2-(trifluoromethyl)benzamide,
721 N-cyclopropyl-N-(3-(4-(cyclopropylamino)-7-methylquinazolin-6-yl)benzyl)benzole sulfonamide,
722 N-cyclopropyl-N-(3-(4-(cyclopropylamino)-7-methylquinazolin-6-yl)benzyl)-3,4-dimethoxybenzamide,
723 N-cyclopropyl-N-(3-(4-(cyclopropylamino)-7-methylquinazolin-6-yl)benzyl)-2-(dimethylamino)acetamide,
724 N-cyclopropyl-N-(3-(4-(cyclopropylamino)-8-fluoroquinazolin-6-yl)benzyl)pivalamide,
725 2,4-dichloro-N-(2-fluorophenyl)-N-(3-(4-(methylamino)quinazolin-6-yl)benzyl)benzole sulfonamide,
726 N-cyclopropyl-2-(dimethylamino)-N-(3-(4-(3-methylbenzylamino)quinazolin-6-yl)benzyl)acetamide,
727 N-cyclopropyl-N-(3-(4-(cyclopropylamino)-8-fluoroquinazolin-6-yl)benzyl)-3-fluorobenzamide,
728 N-cyclopropyl-N-(3-(4-(cyclopropylamino)-8-fluoroquinazolin-6-yl)benzyl)-3-(trifluoromethyl)benzamide,
729 3,5-dichloro-N-(2-fluorophenyl)-N-(3-(4-(methylamino)quinazolin-6-yl)benzyl)benzamide,
730 N-cyclopropyl-N-(3-(4-(2,4-dimethoxybenzylamino)quinazolin-6-yl)benzyl)-1,3-dimethyl-1H-pyrazole-5-carboxamide,
731 N-cyclopropyl-N-((4-(4-(cyclopropylamino)quinazolin-6-yl)pyridin-2-yl)methyl)benzole sulfonamide,
732 N-(2-chloro-5-(4-(cyclopropylamino)quinazolin-6-yl)benzyl)-N-cyclopropyl-3-(trifluoromethyl)benzamide,
733 N-(2-chloro-5-(4-(cyclopropylamino)quinazolin-6-yl)benzyl)-N-cyclopropyl-2-(dimethylamino)acetamide,
734 N-(2-chloro-5-(4-(cyclopropylamino)quinazolin-6-yl)benzyl)-N-cyclopropylthiophene-2-sulfonamide,
735 N-cyclopropyl-N-((4-(4-(cyclopropylamino)quinazolin-6-yl)pyridin-2-yl)methyl)-2-fluoro-4-(trifluoromethyl)benzamide,
736 N-cyclopropyl-N-(3-(4-(cyclopropylamino)-8-fluoroquinazolin-6-yl)benzyl)isonicotinamide,
737 N-cyclopropyl-N-(3-(4-(cyclopropylamino)-7-methylquinazolin-6-yl)benzyl)thiophene-2-sulfonamide,
738 N-cyclopropyl-N-(3-(4-(cyclopropylamino)-8-fluoroquinazolin-6-yl)benzyl)-2-fluoro-4-(trifluoromethyl)benzamide,
739 N-cyclopropyl-N-((4-(4-(cyclopropylamino)quinazolin-6-yl)pyridin-2-yl)methyl)-2-(dimethylamino)acetamide,
740 N-(2-chloro-5-(4-(cyclopropylamino)quinazolin-6-yl)benzyl)-N-cyclopropyl-2-(trifluoromethyl)benzamide,
741 N-(2-chloro-5-(4-(cyclopropylamino)quinazolin-6-yl)benzyl)-N-cyclopropylcyclohexane carboxamide,
742 N-(2-chloro-5-(4-(cyclopropylamino)quinazolin-6-yl)benzyl)-N-cyclopropylquinoline-8-sulfonamide,
743 N-cyclopropyl-N-((4-(4-(cyclopropylamino)quinazolin-6-yl)pyridin-2-yl)methyl)-3-(trifluoromethyl)benzamide,
744 N-cyclopropyl-N-(3-(4-(cyclopropylamino)-8-fluoroquinazolin-6-yl)benzyl)-2-methoxyacetamide,
745 N-cyclopropyl-N-(3-(4-(cyclopropylamino)-7-methylquinazolin-6-yl)benzyl)isoxazole-5-carboxamide,
746 N-(2-chloro-5-(4-(cyclopropylamino)quinazolin-6-yl)benzyl)-N-cyclopropylfuran-2-carboxamide,
747 3,5-dichloro-N-(2-chloro-5-(4-(cyclopropylamino)quinazolin-6-yl)benzyl)-N-cyclopropylbenzamide,
748 N-(2-chloro-5-(4-(cyclopropylamino)quinazolin-6-yl)benzyl)-N-cyclopropyl-2-phenylacetamide,
749 N-cyclopropyl-N-((4-(4-(cyclopropylamino)quinazolin-6-yl)pyridin-2-yl)methyl)-2-(trifluoromethyl)benzamide,
750 3,5-dichloro-N-cyclopropyl-N-((4-(4-(cyclopropylamino)quinazolin-6-yl)pyridin-2-yl)methyl)benzamide,
751 N-cyclopropyl-N-(3-(4-(cyclopropylamino)-8-fluoroquinazolin-6-yl)benzyl)-2-(dimethylamino)acetamide,
752 N-cyclopropyl-N-(3-(4-(cyclopropylamino)-8-fluoroquinazolin-6-yl)benzyl)-2,3,4,5,6-pentafluorobenzamide,
753 N-cyclopropyl-N-(3-(4-(cyclopropylamino)-8-fluoroquinazolin-6-yl)benzyl)-1-phenylmethane sulfonamide,
754 N-(2-chloro-5-(4-(cyclopropylamino)quinazolin-6-yl)benzyl)-N-cyclopropylthiophene-2-carboxamide,
755 N-(2-chloro-5-(4-(cyclopropylamino)quinazolin-6-yl)benzyl)-N-cyclopropylpicolinamide,
756 N-(2-chloro-5-(4-(cyclopropylamino)quinazolin-6-yl)benzyl)-N-cyclopropyl-3-phenylpropanamide,
757 N-cyclopropyl-N-((4-(4-(cyclopropylamino)quinazolin-6-yl)pyridin-2-yl)methyl)furan-2-carboxamide,
758 N-cyclopropyl-N-((4-(4-(cyclopropylamino)quinazolin-6-yl)pyridin-2-yl)methyl)picolinamide,
759 N-cyclopropyl-N-(3-(4-(cyclopropylamino)-7-methylquinazolin-6-yl)benzyl)-2-methylthiazole-4-carboxamide,
760 N-cyclopropyl-N-(3-(4-(cyclopropylamino)-8-fluoroquinazolin-6-yl)benzyl)-1,3-dimethyl-1H-pyrazole-5-carboxamide,
761 N-cyclopropyl-N-(3-(4-(cyclopropylamino)-8-fluoroquinazolin-6-yl)benzyl)-2,3,4,5,6-pentafluorobenzole sulfonamide,
762 N-(2-chloro-5-(4-(cyclopropylamino)quinazolin-6-yl)benzyl)-N-cyclopropyl isoxazole-5-carboxamide,
763 N-(2-chloro-5-(4-(cyclopropylamino)quinazolin-6-yl)benzyl)-N-cyclopropyl isonicotinamide,
764 N-(2-chloro-5-(4-(cyclopropylamino)quinazolin-6-yl)benzyl)-N-cyclopropyl methane sulfonamide,
765 N-cyclopropyl-N-((4-(4-(cyclopropylamino)quinazolin-6-yl)pyridin-2-yl)methyl)isoxazole-5-carboxamide,
766 N-cyclopropyl-N-((4-(4-(cyclopropylamino)quinazolin-6-yl)pyridin-2-yl)methyl)nicotinamide,
767 N-(2-fluorophenyl)-N-(3-(4-(methylamino)quinazolin-6-yl)benzyl)furan-2-carboxamide,
768 N-cyclopropyl-N-(3-(4-(cyclopropylamino)-7-methylquinazolin-6-yl)benzyl)-2,3,4,5,6-pentafluorobenzamide,
769 N-(2-chloro-5-(4-(cyclopropylamino)quinazolin-6-yl)benzyl)-N-cyclopropyl-2-methylthiazole-4-carboxamide,
770 N-(2-chloro-5-(4-(cyclopropylamino)quinazolin-6-yl)benzyl)-N-cyclopropyl nicotinamide,
771 N-(2-chloro-5-(4-(cyclopropylamino)quinazolin-6-yl)benzyl)-N-cyclopropyl-1-phenylmethane sulfonamide,
772 N-cyclopropyl-N-((4-(4-(cyclopropylamino)quinazolin-6-yl)pyridin-2-yl)methyl)-2-methylthiazole-4-carboxamide,
773 N-cyclopropyl-N-((4-(4-(cyclopropylamino)quinazolin-6-yl)pyridin-2-yl)methyl)acetamide,
774 3,5-dichloro-N-cyclopropyl-N-(3-(4-(cyclopropylamino)-8-fluoroquinazolin-6-yl)benzyl)benzamide,
775 N-cyclopropyl-N-(3-(4-(cyclopropylamino)-8-fluoroquinazolin-6-yl)benzyl)isoxazole-5-carboxamide,
776 N-(2-chloro-5-(4-(cyclopropylamino)quinazolin-6-yl)benzyl)-N-cyclopropyl-1,3-dimethyl-1H-pyrazole-5-carboxamide,
777 N-(2-chloro-5-(4-(cyclopropylamino)quinazolin-6-yl)benzyl)-N-cyclopropyl acetamide,
778 N-(2-chloro-5-(4-(cyclopropylamino)quinazolin-6-yl)benzyl)-N-cyclopropyl-3-(trifluoromethyl)benzole sulfonamide,
779 N-cyclopropyl-N-((4-(4-(cyclopropylamino)quinazolin-6-yl)pyridin-2-yl)methyl)-1,3-dimethyl-1H-pyrazole-5-carboxamide,
780 N-cyclopropyl-N-((4-(4-(cyclopropylamino)quinazolin-6-yl)pyridin-2-yl)methyl)-2-methoxyacetamide,
781 N-cyclopropyl-N-(3-(4-(cyclopropylamino)-8-fluoroquinazolin-6-yl)benzyl)nicotinamide,
782 2,4-dichloro-N-cyclopropyl-N-(3-(4-(cyclopropylamino)-8-fluoroquinazolin-6-yl)benzyl)benzole sulfonamide,
783 N-(2-chloro-5-(4-(cyclopropylamino)quinazolin-6-yl)benzyl)-N-cyclopropyl benzamide,
784 N-(2-chloro-5-(4-(cyclopropylamino)quinazolin-6-yl)benzyl)-N-cyclopropyl-3,4-dimethoxybenzamide,
785 N-(2-chloro-5-(4-(cyclopropylamino)quinazolin-6-yl)benzyl)-N-cyclopropyl pivalamide,
786 N-(2-chloro-5-(4-(cyclopropylamino)quinazolin-6-yl)benzyl)-N-cyclopropyl-4-methoxybenzole sulfonamide,
787 N-cyclopropyl-N-((4-(4-(cyclopropylamino)quinazolin-6-yl)pyridin-2-yl)methyl)-3,4-dimethoxybenzamide,
788 N-cyclopropyl-N-((4-(4-(cyclopropylamino)quinazolin-6-yl)pyridin-2-yl)methyl)thiophene-2-sulfonamide,
789 N-cyclopropyl-N-(3-(4-(cyclopropylamino)-8-fluoroquinazolin-6-yl)benzyl)-2-(trifluoromethyl)benzamide,
790 2-(dimethylamino)-N-(3-(4-(methylamino)quinazolin-6-yl)benzyl)-N-(pyridin-2-ylmethyl)acetamide,
791 N-(2-chloro-5-(4-(cyclopropylamino)quinazolin-6-yl)benzyl)-N-cyclopropyl-3-fluorobenzamide,
792 N-(2-chloro-5-(4-(cyclopropylamino)quinazolin-6-yl)benzyl)-N-cyclopropyl-2,4-difluorobenzamide,
793 N-(2-chloro-5-(4-(cyclopropylamino)quinazolin-6-yl)benzyl)-N-cyclopropyl pentanamide,
794 2,4-dichloro-N-(2-chloro-5-(4-(cyclopropylamino)quinazolin-6-yl)benzyl)-N-cyclopropyl benzole sulfonamide,
795 N-cyclopropyl-N-((4-(4-(cyclopropylamino)quinazolin-6-yl)pyridin-2-yl)methyl)-3-fluorobenzamide,
796 2,4-dichloro-N-cyclopropyl-N-((4-(4-(cyclopropylamino)quinazolin-6-yl)pyridin-2-yl)methyl)benzole sulfonamide,
797 N-cyclopropyl-N-(3-(4-(cyclopropylamino)-7-methylquinazolin-6-yl)benzyl)-2-methoxyacetamide,
798 N-cyclopropyl-2-methoxy-N-(3-(4-(phenethylamino)quinazolin-6-yl)benzyl)acetamide,
799 N-(2-chloro-5-(4-(cyclopropylamino)quinazolin-6-yl)benzyl)-N-cyclopropyl benzole sulfonamide,
800 N-(2-chloro-5-(4-(cyclopropylamino)quinazolin-6-yl)benzyl)-N-cyclopropyl-2-fluoro-4-(trifluoromethyl)benzamide,
801 N-(2-chloro-5-(4-(cyclopropylamino)quinazolin-6-yl)benzyl)-N-cyclopropyl-2-methoxyacetamide,
802 N-(2-chloro-5-(4-(cyclopropylamino)quinazolin-6-yl)benzyl)-N-cyclopropyl-2,3,4,5,6-pentafluorobenzole sulfonamide,
803 N-cyclopropyl-N-((4-(4-(cyclopropylamino)quinazolin-6-yl)pyridin-2-yl)methyl)-2,3,4,5,6-pentafluorobenzamide,
804 N-cyclopropyl-N-((4-(4-(cyclopropylamino)quinazolin-6-yl)pyridin-2-yl)methyl)propane-2-sulfonamide,
805 N-cyclopropyl-N-((4-(4-(cyclopropylamino)quinazolin-6-yl)pyridin-2-yl)methyl)-1-phenylmethane sulfonamide,
806 N-cyclopropyl-N-((4-(4-(cyclopropylamino)quinazolin-6-yl)pyridin-2-yl)methyl)-4-methoxybenzole sulfonamide,
807 N-(5-(4-(tert-butylamino)quinazolin-6-yl)-2-fluorobenzyl)-N-cyclopropyl furan-2-carboxamide,
808 N-(5-(4-(tert-butylamino)quinazolin-6-yl)-2-fluorobenzyl)-N-cyclopropyl-3-(trifluoromethyl)benzamide,
809 N-(5-(4-(tert-butylamino)quinazolin-6-yl)-2-fluorobenzyl)-N-cyclopropyl cyclohexane carboxamide,
810 N-(5-(4-(tert-butylamino)quinazolin-6-yl)-2-fluorobenzyl)-N-cyclopropyl thiophene-2-sulfonamide,
811 N-cyclopropyl-N-((4-(4-(cyclopropylamino)quinazolin-6-yl)pyridin-2-yl)methyl)thiophene-2-carboxamide,
812 N-cyclopropyl-N-((4-(4-(cyclopropylamino)quinazolin-6-yl)pyridin-2-yl)methyl)quinoline-8-sulfonamide,
813 N-cyclopropyl-N-((4-(4-(cyclopropylamino)quinazolin-6-yl)pyridin-2-yl)methyl)benzamide,
814 N-(5-(4-(tert-butylamino)quinazolin-6-yl)-2-fluorobenzyl)-N-cyclopropyl-2-(trifluoromethyl)benzamide,
815 N-(5-(4-(tert-butylamino)quinazolin-6-yl)-2-fluorobenzyl)-N-cyclopropyl-2-phenylacetamide,
816 N-(5-(4-(tert-butylamino)quinazolin-6-yl)-2-fluorobenzyl)-N-cyclopropyl quinoline-8-sulfonamide,
817 N-cyclopropyl-N-((4-(4-(cyclopropylamino)quinazolin-6-yl)pyridin-2-yl)methyl)-2,4-difluorobenzamide,
818 N-cyclopropyl-N-(3-(4-(phenethylamino)quinazolin-6-yl)benzyl)propane-2-sulfonamide,
819 N-(5-(4-(tert-butylamino)quinazolin-6-yl)-2-fluorobenzyl)-N-cyclopropyl thiophene-2-carboxamide,
820 N-(5-(4-(tert-butylamino)quinazolin-6-yl)-2-fluorobenzyl)-3,5-dichloro-N-cyclopropyl benzamide,
821 N-(5-(4-(tert-butylamino)quinazolin-6-yl)-2-fluorobenzyl)-N-cyclopropyl-3-phenyl propanamide,
822 N-(5-(4-(tert-butylamino)quinazolin-6-yl)-2-fluorobenzyl)-N-cyclopropyl propane-2-sulfonamide,
823 N-cyclopropyl-N-((4-(4-(cyclopropylamino)quinazolin-6-yl)pyridin-2-yl)methyl)isonicotinamide,
824 N-cyclopropyl-N-(3-(4-(cyclopropylamino)-8-fluoroquinazolin-6-yl)benzyl)propane-2-sulfonamide,
825 N-(5-(4-(tert-butylamino)quinazolin-6-yl)-2-fluorobenzyl)-N-cyclopropyl-2-methylthiazole-4-carboxamide,
826 N-(5-(4-(tert-butylamino)quinazolin-6-yl)-2-fluorobenzyl)-N-cyclopropyl picolinamide,
827 N-(5-(4-(tert-butylamino)quinazolin-6-yl)-2-fluorobenzyl)-N-cyclopropyl methane sulfonamide,
828 N-cyclopropyl-N-((4-(4-(cyclopropylamino)quinazolin-6-yl)pyridin-2-yl)methyl)pivalamide,
829 N-cyclopropyl-N-((4-(4-(cyclopropylamino)quinazolin-6-yl)pyridin-2-yl)methyl)pentanamide,
830 N-cyclopropyl-N-(3-(4-(cyclopropylamino)-8-fluoroquinazolin-6-yl)benzyl)-2-methylthiazole-4-carboxamide,
831 N-(5-(4-(tert-butylamino)quinazolin-6-yl)-2-fluorobenzyl)-N-cyclopropyl-1,3-dimethyl-1H-pyrazole-5-carboxamide,
832 N-(5-(4-(tert-butylamino)quinazolin-6-yl)-2-fluorobenzyl)-N-cyclopropyl isonicotinamide,
833 N-(5-(4-(tert-butylamino)quinazolin-6-yl)-2-fluorobenzyl)-N-cyclopropyl-1-phenyl methane sulfonamide,
834 N-(5-(4-(tert-butylamino)quinazolin-6-yl)-2-fluorobenzyl)-N-cyclopropyl isoxazole-5-carboxamide,
835 N-cyclopropyl-N-((4-(4-(cyclopropylamino)quinazolin-6-yl)pyridin-2-yl)methyl)cyclohexane carboxamide,
836 N-cyclopropyl-N-(3-(4-(2,4-dimethoxybenzylamino)quinazolin-6-yl)benzyl)propane-2-sulfonamide,
837 N-(5-(4-(tert-butylamino)quinazolin-6-yl)-2-fluorobenzyl)-N-cyclopropyl benzamide,
838 N-(5-(4-(tert-butylamino)quinazolin-6-yl)-2-fluorobenzyl)-N-cyclopropyl nicotinamide,
839 N-(5-(4-(tert-butylamino)quinazolin-6-yl)-2-fluorobenzyl)-N-cyclopropyl benzole sulfonamide,
840 N-(5-(4-(tert-butylamino)quinazolin-6-yl)-2-fluorobenzyl)-N-cyclopropyl-2,3,4,5,6-pentafluorobenzamide,
841 N-cyclopropyl-N-((4-(4-(cyclopropylamino)quinazolin-6-yl)pyridin-2-yl)methyl)-2-phenylacetamide,
842 N-cyclopropyl-N-(3-(4-(2,4-dimethoxybenzylamino)quinazolin-6-yl)benzyl)-2,3,4,5,6-pentafluorobenzole sulfonamide,
843 N-(5-(4-(tert-butylamino)quinazolin-6-yl)-2-fluorobenzyl)-N-cyclopropyl-3,4-dimethoxybenzamide,
844 N-(5-(4-(tert-butylamino)quinazolin-6-yl)-2-fluorobenzyl)-N-cyclopropyl acetamide,
845 N-(5-(4-(tert-butylamino)quinazolin-6-yl)-2-fluorobenzyl)-N-cyclopropyl-3-(trifluoromethyl)benzole sulfonamide,
846 N-(5-(4-(tert-butylamino)quinazolin-6-yl)-2-fluorobenzyl)-N-cyclopropyl-2-methoxyacetamide,
847 N-cyclopropyl-N-((4-(4-(cyclopropylamino)quinazolin-6-yl)pyridin-2-yl)methyl)-3-phenylpropanamide,
848 N-cyclopropyl-N-((4-(4-(cyclopropylamino)quinazolin-6-yl)pyridin-2-yl)methyl)-2,3,4,5,6-pentafluorobenzole sulfonamide,
849 N-(5-(4-(tert-butylamino)quinazolin-6-yl)-2-fluorobenzyl)-N-cyclopropyl-3-fluorobenzamide,
850 N-(5-(4-(tert-butylamino)quinazolin-6-yl)-2-fluorobenzyl)-N-cyclopropyl pivalamide,
851 N-(5-(4-(tert-butylamino)quinazolin-6-yl)-2-fluorobenzyl)-N-cyclopropyl-4-methoxybenzole sulfonamide,
852 N-(2-fluorophenyl)-N-(3-(4-(methylamino)quinazolin-6-yl)benzyl)benzole sulfonamide,
853 N-cyclopropyl-N-((4-(4-(cyclopropylamino)quinazolin-6-yl)pyridin-2-yl)methyl)methane sulfonamide,
854 N-(2-chloro-5-(4-(cyclopropylamino)quinazolin-6-yl)benzyl)-N-cyclopropyl propane-2-sulfonamide,
855 N-(5-(4-(tert-butylamino)quinazolin-6-yl)-2-fluorobenzyl)-N-cyclopropyl-2,4-difluorobenzamide,
856 N-(5-(4-(tert-butylamino)quinazolin-6-yl)-2-fluorobenzyl)-N-cyclopropyl pentanamide,
857 N-(5-(4-(tert-butylamino)quinazolin-6-yl)-2-fluorobenzyl)-2,4-dichloro-N-cyclopropyl benzole sulfonamide,
858 N-cyclopropyl-N-((4-(4-(cyclopropylamino)quinazolin-6-yl)pyridin-2-yl)methyl)-3-(trifluoromethyl)benzole sulfonamide,
859 N-(2-chloro-5-(4-(cyclopropylamino)quinazolin-6-yl)benzyl)-N-cyclopropyl-2,3,4,5,6-pentafluorobenzamide,
860 N-(5-(4-(tert-butylamino)quinazolin-6-yl)-2-fluorobenzyl)-N-cyclopropyl-2-fluoro-4-(trifluoromethyl)benzamide,
861 N-(5-(4-(tert-butylamino)quinazolin-6-yl)-2-fluorobenzyl)-N-cyclopropyl-2-(dimethylamino)acetamide,
862 N-(5-(4-(tert-butylamino)quinazolin-6-yl)-2-fluorobenzyl)-N-cyclopropyl-2,3,4,5,6-pentafluorobenzole sulfonamide,
866 1-(3-(4-(neopentylamino)quinazolin-6-yl)benzyl)pyrrolidine-2,5-dione,
867 1-(3-(4-(2-methoxyethylamino)quinazolin-6-yl)benzyl)pyrrolidine-2,5-dione hydrochloride,
868 N-cyclopropyl-N-(3-(4-(cyclopropylamino)quinazolin-6-yl)benzyl)-1-methylpiperidine-4-carboxamide,
869 1-amino-N-(3-(4-(cyclopropylamino)quinazolin-6-yl)benzyl)cyclopropane carboxamide dihydrochloride,
870 N-cyclopropyl-N-(5-(4-(cyclopropylamino)quinazolin-6-yl)-2-fluorobenzyl)-2,2,2-trifluoroacetamide,
871 1-(3-(4-morpholinoquinazolin-6-yl)benzyl)pyrrolidine-2,5-dione,
872 1-(3-(4-(cyclobutylamino)quinazolin-6-yl)benzyl)pyrrolidine-2,5-dione hydrochloride,
873 1-(3-(4-(isopropylamino)quinazolin-6-yl)benzyl)pyrrolidine-2,5-dione hydrochloride,
874 N-(3-(4-(cyclopropylamino)quinazolin-6-yl)benzyl)-1-pentanamidocyclopropane carboxamide,
875 ethyl cyclopropyl(3-(4-(cyclopropylamino)quinazolin-6-yl)benzyl)carbamate,
876 6-(3-((1H-pyrrol-1-yl)methyl)phenyl)-N-cyclopropylquinazoline-4-amine,
877 1-(3-(4-(cyclopropylamino)quinazolin-6-yl)benzyl)-3-methylimidazolidin-2-one,
878 N-cyclopropyl-6-(3-((methyl(pyridin-2-yl)amino)methyl)phenyl)quinazoline-4-amine,
879 N-acetyl-N-(3-(4-(cyclopropylamino)quinazolin-6-yl)benzyl)acetamide,
880 1-(3-(4-(pyrrolidin-1-yl)quinazolin-6-yl)benzyl)pyrrolidine-2,5-dione,
881 1-(3-(8-chloro-4-(cyclopropylamino)quinazolin-6-yl)benzyl)pyrrolidine-2,5-dione,
882 1-(3-(4-(cyclopropylamino)-7-fluoroquinazolin-6-yl)benzyl)pyrrolidine-2,5-dione,
883 1-(3-(4-(cyclopropylamino)-8-(trifluoromethyl)quinazolin-6-yl)benzyl)pyrrolidine-2,5-dione,
884 1-(3-(4-(cyclopropylamino)-7-methylquinazolin-6-yl)benzyl)pyrrolidine-2,5-dione,
885 1-(3-(4-(cyclopropylamino)quinazolin-6-yl)benzyl)-5-methylpyrrolidine-2-one,
886 N-allyl-N-(3-(4-(cyclopropylamino)quinazolin-6-yl)benzyl)acetamide,
887 N-(3-(4-(cyclopropylamino)quinazolin-6-yl)benzyl)-N-propylacetamide hydrochloride,
888 N-cyclopropyl-6-(3-((cyclopropyl(pyridin-2-yl)amino)methyl)phenyl)quinazoline-4-amine,
889 1-(3-(4-(cyclopropylamino)quinazolin-6-yl)-5-fluorobenzyl)pyrrolidine-2,5-dione,
890 N-cyclopropyl-N-((6-(4-(cyclopropylamino)quinazolin-6-yl)pyridin-2-yl)methyl)acetamide,
891 N-cyclopropyl-6-(3-((cyclopropyl(methyl)amino)methyl)phenyl)quinazoline-4-amine,
892 N-cyclopropyl-N-(3-(4-(cyclopropylamino)quinazolin-6-yl)benzyl)thiazole-2-amine,
893 N-cyclobutyl-N-(3-(4-(cyclopropylamino)quinazolin-6-yl)benzyl)acetamide,
894 N-(3-(4-(cyclopropylamino)quinazolin-6-yl)benzyl)-N-ethylacetamide,
895 1-(3-(4-(cyclopropylamino)quinazolin-6-yl)-4-fluorobenzyl)pyrrolidine-2,5-dione,
896 N-cyclopropyl-N-(3-(4-(cyclopropylamino)quinazolin-6-yl)-4-fluorobenzyl)acetamide,
897 N-cyclopropyl-N-(3-(4-(cyclopropylamino)quinazolin-6-yl)-5-fluorobenzyl)acetamide,
898 methyl cyclopropyl(3-(4-(cyclopropylamino)quinazolin-6-yl)benzyl)carbamate,
899 1-(3-(4-(cyclopropylamino)-7-methoxyquinazolin-6-yl)benzyl)pyrrolidine-2,5-dione,
900 2-cyano-N-cyclopropyl-N-(3-(4-(cyclopropylamino)quinazolin-6-yl)-4-fluorobenzyl)acetamide,
901 1-(3-(4-(cyclopropyl(methyl)amino)quinazolin-6-yl)benzyl)pyrrolidine-2,5-dione,
902 2-cyano-N-cyclopropyl-N-(3-(4-(cyclopropylamino)quinazolin-6-yl)-5-fluorobenzyl)acetamide,
903 N-cyclopropyl-N-((4-(4-(cyclopropylamino)quinazolin-6-yl)thiazole-2-yl)methyl)acetamide,
904 1-(3-(4-(cyclopropylamino)-5-fluoroquinazolin-6-yl)benzyl)pyrrolidine-2,5-dione,
905 2-cyano-N-cyclopropyl-N-(5-(4-(cyclopropylamino)-7-fluoroquinazolin-6-yl)-2-fluorobenzyl)acetamide,
906 2-cyano-N-cyclopropyl-N-(3-(4-(cyclopropylamino)-7-fluoroquinazolin-6-yl)benzyl)acetamide,
907 1-(3-(4-(cyclopropylamino)-8-fluoroquinazolin-6-yl)benzyl)pyrrolidine-2,5-dione,
908 N-cyclopropyl-6-(3-((cyclopropyl(phenyl)amino)methyl)phenyl)quinazoline-4-amine,
909 1-((4-(4-(cyclopropylamino)quinazolin-6-yl)thiophen-2-yl)methyl)pyrrolidine-2,5-dione,
910 1-(3-(4-(cyclopropylamino)-5,7-difluoroquinazolin-6-yl)benzyl)pyrrolidine-2,5-dione,
911 N-cyclopropyl-N-(3-(4-(cyclopropylamino)quinazolin-6-yl)-2-methylbenzyl)acetamide, and
912 N-cyclopropyl-N-(5-(4-(cyclopropylmethyl)quinazolin-6-yl)-2-methylbenzyl)acetamide,
   in each case optionally in the form of one of their pure stereoisomers, in particular enantiomers or diastereomers, their racemates, or in the form of a mixture of stereoisomers, in particular the enantiomers and/or diastereomers, in any mixture ratio, or respectively in the form of corresponding salts or respectively in the form of corresponding solvates.

The present invention additionally relates to a method for producing compounds corresponding to the foregoing formula I, said method comprising reacting a compound corresponding to formula II

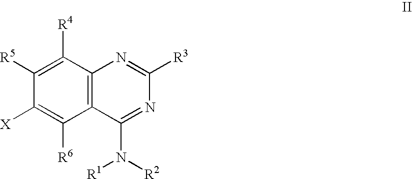

wherein R¹, R², R³, R⁴, R⁵ and R⁶ have the meanings specified above and X represents a leaving group, preferably a halogen residue or a sulfonic ester, particularly preferably a leaving group selected from the group consisting of chlorine, bromine, iodine, triflate, mesylate and tosylate,
with a compound corresponding to formula III

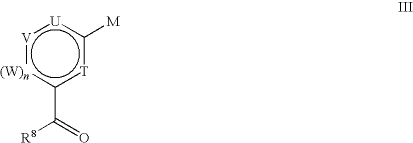

wherein T, U, V, W, n and R⁸ have the meanings specified above and M represents —MgY or —ZnY, wherein Y represents a halogen residue or a sulfonic ester, preferably chlorine, bromine, iodine, mesylate or triflate, or M represents —BF₃K, —B(OH)₂ or —B(OR^{A})₂, wherein R^{A} represents alkyl or two residues R^{A} together with the —O—B—O— group linking them form a heterocycloalkyl residue, preferably together with the —O—B—O— group linking them form a 1,3,2-dioxaborolan-2-yl residue,
optionally in a reaction medium, preferably in a reaction medium selected from the group consisting of methanol, ethyl acetate, ethanol, isopropanol, diethyl ether, dioxane, tetrahydrofuran, dimethylformamide, dimethoxyethane, acetonitrile, dimethyl sulfoxide, toluene, N-methyl-pyrrolidine and water or mixtures thereof, optionally in the presence of a base, preferably in the presence of a base selected from the group consisting of potassium carbonate, sodium carbonate, potassium phosphate, sodium hydrogenphosphate, cesium carbonate, triethylamine, [1,4]-diazabicyclo-[2,2]-octane, diisopropylamine, diisopropylethylamine and N-methylmorpholine, in the presence of a catalyst, which optionally may be polymer-bonded, preferably in the presence of a catalyst, which may be polymer-bonded, selected from the group consisting of palladium(II)acetate, tri(dibenzylideneacetone)dipalladium, palladium(0)bis-dibenzylideneacetone), tetrakis(triphenylphosphine)palladium(0), (1,1′-bis(diphenyl-phosphino)ferrocene)-dichloropalladium(II), bis(acetonitrile)dichloropalladium(II), palladium(II)chloride, dichlorobis(triphenylphosphine)palladium(II), dichloro(tricyclohexylphosphine)-palladium(II), bis(acetato)bis(triphenylphosphine)palladium(II), bistriphenylphosphine-palladium(II)dichloride, bistriphenylphosphine-palladium(II)acetate and iron(III)chloride, optionally in the presence of a ligand, which can be polymer-bonded, preferably in the presence of a ligand, which can be polymer-bonded, selected from the group consisting of 2-dicyclohexylphosphino-2′,6′-dimethoxybiphenyl (S-phos), 2-dicyclohexylphosphino-2′,4′,6′-triisopropylbiphenyl (X-phos), tricyclohexylphosphine, tricyclohexylphosphine tetrafluoroborate, tri-tert-butylphosphine tetrafluoroborate, triphenylphosphine and imidazolium salts, preferably at a temperature of −70° C. to 300° C.,
to yield a compound corresponding to formula IV

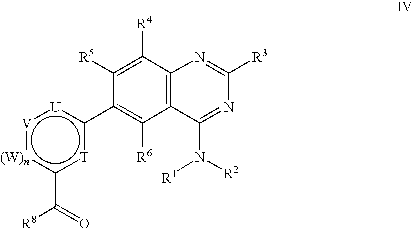

wherein R¹, R², R³, R⁴, R⁵, R⁶, R⁸, T, U, V, W and n have the meanings specified above, and optionally purifying and/or isolating the compound of formula IV; and
reacting the compound of formula IV in a reaction medium, preferably in a reaction medium selected from the group consisting of diethyl ether, toluene, tetrahydrofuran, dichloromethane, methanol and ethanol, or a mixture thereof, in the presence of a reducing agent, which optionally may be polymer-bonded, preferably in the presence of a reducing agent, which may be polymer-bonded, selected from the group consisting of sodium borohydride, sodium triacetoxyborohydride, borane, diisobutyl aluminium hydride and red-Al, preferably at a temperature or −100° C. to 200° C.,
to a compound corresponding to formula V

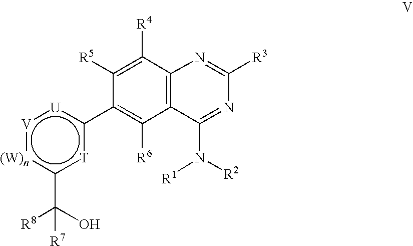

wherein R¹, R², R³, R⁴, R⁵, R⁶, R⁸, T, U, V, W and n have the meanings specified above and R⁷ represents H,
and optionally purifying or isolating the compound of formula V; and
reacting the compound of formula V with a compound corresponding to the formula: HNR⁹R¹⁰ wherein R⁹ and R¹⁰ have the meanings specified above,
optionally in a reaction medium, preferably in a reaction medium selected from the group consisting of tetrahydrofuran, dioxane and dichloromethane or mixtures thereof,
in the presence of a compound corresponding to the formula:

R^{B}—O—C(═O)—N═N—C(═O)—O—R^{B}

which optionally may be polymer-bonded, wherein R^{B} represents alkyl or benzyl, preferably in the presence of a compound selected from the group consisting of diethylazodicarboxylate, di-tert-butyl-azodicarboxylate, diisopropyl azodicarboxylate and polymer-bonded diethyl azodicarboxylate, in the presence of at least one tertiary phosphine, which optionally may be polymer-bonded, preferably in the presence of a tertiary phosphine selected from the group consisting of triphenylphosphine, polymer-bonded triphenylphosphine and fluorinated triphenylphosphine, preferably at a temperature of −100° C. to 200° C., to yield a compound corresponding to formula I

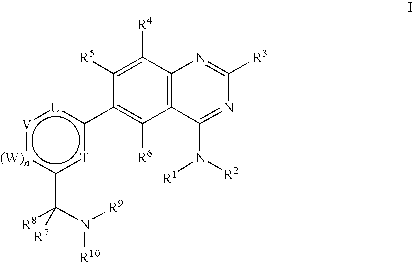

wherein the meaning of R¹, R², R³, R⁴, R⁵, R⁶, R⁸, R⁹, R¹⁰, T, U, V, W and n is as specified above and R⁷ represents H,
and this is optionally purified and/or isolated; or
at least one compound of the general formula IV is reacted with at least one compound of the general formula H₂NR⁹, wherein the meaning of R⁹ is as specified above, in at least one reaction medium, preferably in at least one reaction medium selected from the group consisting of tetrahydrofuran, dioxane, dichloromethane, methanol and ethanol, or corresponding mixtures, in the presence of at least one reducing agent, which can be polymer-bonded, preferably in the presence of at least one reducing agent, which can be polymer-bonded, selected from the group consisting of sodium triacetoxyborohydride, sodium cyanoborohydride and sodium diacetoxyborohydride,
or in the presence of at least one catalyst, preferably in the presence of palladium on carbon or in the presence of a rhodium catalyst, in a hydrogen atmosphere, preferably at a temperature of −100° C. to 200° C., into at least one corresponding compound of the general formula I, wherein the meaning of R¹, R², R³, R⁴, R⁵, R⁶, R⁸, R⁹, T, U, V, W and n is as specified above and R⁷ and R¹⁰ each represent H,
and optionally purifying or isolating this product; and
optionally at least one compound of the general formula I, wherein R¹, R², R³, R⁴, R⁵, R⁶, R⁸, R⁹, T, U, V, W and n have the meanings specified above and R⁷ and R¹⁰ each represent H, is reacted with at least one compound of the formula Z-S(═O)₂—R²¹, wherein R²¹ has the meaning specified above and Z represents a leaving group, preferably a halogen residue, particularly preferably a chlorine atom, optionally in at least one reaction medium, preferably in at least one reaction medium selected from the group consisting of tetrahydrofuran, dioxane, dichloromethane, diethyl ether, toluene, acetonitrile and dimethylformamide or mixtures thereof, optionally in the presence of a base, preferably in the presence of a base selected from the group consisting of triethylamine, pyridine, diisopropylethylamine, dimethylaminopyridine, N-methyl-morpholine and diisopropylamine, preferably at a temperature of −70° C. to 200° C., to yield a compound corresponding to formula I, wherein R¹, R², R³, R⁴, R⁵, R⁶, R⁸, R⁹, T, U, V, W and n have the meanings specified above and R⁷ represents H and R¹⁰ represents —S(═O)₂—R²¹,
and optionally purifying or isolating this product; or
optionally reacting a compound corresponding to formula I, wherein R¹, R², R³, R⁴, R⁵, R⁶, R⁸, R⁹, T, U, V, W and n have the meanings specified above and R⁷ and R¹⁰ each denote H, with a compound of the formula Z-C(═O)—R¹⁵, wherein R¹⁵ has the meaning specified above and Z represents a leaving group, preferably a halogen residue, particularly preferably a chlorine atom, optionally in a reaction medium, preferably in a reaction medium selected from the group consisting of tetrahydrofuran, dioxane, dichloromethane, diethyl ether, toluene, acetonitrile and dimethylformamide, or mixtures thereof, optionally in the presence of at least one base, preferably in the presence of at least one base selected from the group consisting of triethylamine, pyridine, dimethylaminopyridine, diisopropylethylamine, N-methyl-morpholine and diisopropylamine, preferably at a temperature of −70° C. to 200° C., into at least one compound of the general formula I, wherein the meaning of R¹, R², R³, R⁴, R⁵, R⁶, R⁸, R⁹, T, U, V, W and n is as specified above and R⁷ represents H and R¹⁰ represents —C(═O)—R¹⁵,
and this is optionally purified and/or isolated; or
or optionally at least one compound of the general formula I, wherein the meaning of R¹, R², R³, R⁴, R⁵, R⁶, R⁸, R⁹, T, U, V, W and n is as specified above and R⁷ and R¹⁰ respectively represent H, is reacted with at least one compound of the general formula OH—C(═O)—R¹⁵, wherein the meaning of R¹⁵ is as specified above, possibly in at least one reaction medium, preferably at least one reaction medium selected from the group consisting of tetrahydrofuran, dioxane, dichloromethane, diethyl ether, toluene, acetonitrile and dimethylformamide, or corresponding mixtures, possibly in the presence of at least one base, preferably in the presence of at least one base selected from the group consisting of triethylamine, pyridine, diisopropylethylamine, dimethylaminopyridine, N-methyl-morpholine and diisopropylamine, in the presence of at least one coupling reagent, preferably in the presence of at least one coupling reagent selected from the group consisting of 1-benzotriazolyloxy-tris-(dimethylamino)-phosphonium hexafluorophosphate (BOP), dicyclohexylcarbodiimide (DCC), N′-(3-dimethylaminopropyl)-N-ethylcarbodiimide (EDCI), diisopropylcarbodiimide, 1,1′-carbonyl-diimidazole (CDI), N-[(dimethylamino)-1H-1,2,3-triazolo[4,5-b]pyridino-1-ylmethylene]-N-methylmethanaminium hexafluorophosphate N-oxide (HATU), O-(benzotriazol-1-yl)-N,N,N′,N′-tetramethyluronium hexafluorophosphate (HBTU), O-(benzotriazol-1-yl)-N,N,N′,N′-tetramethyluronium-tetrafluoroborate (TBTU), 1-hydroxy-1H-benzotriazole (HOBT), pentafluorophenyl trifluoroacetate and 1-hydroxy-7-azabenzotriazole (HOAt), which can respectively be polymer-bonded, preferably at a temperature of −70° C. to 200° C., into at least one compound of the general formula I, wherein the meaning of R¹, R², R³, R⁴, R⁵, R⁶, R⁸, R⁹, T, U, V, W and n is as specified above and R⁷ represents H and R¹⁰ represents —C(═O)—R¹⁵,
and this is optionally purified and/or isolated; or
optionally at least one compound of the general formula I, wherein R¹, R², R³, R⁴, R⁵, R⁶, R⁸, R⁹, T, U, V, W and n have the meanings specified above and R⁷ and R¹⁰ respectively represent H, is reacted with at least one compound of the general formula R¹⁷—N═C═O or with at least one compound of the general formula R¹⁷—N═C═S, wherein the meaning of R¹⁷ is as specified above, possibly in at least one reaction medium, preferably in at least one reaction medium selected from the group consisting of tetrahydrofuran, dioxane, dichloromethane, diethyl ether, toluene, acetonitrile and dimethylformamide or corresponding mixtures, possibly in the presence of at least one base, preferably in the presence of at least one base selected from the group consisting of triethylamine, pyridine, diisopropylethylamine, dimethylaminopyridine, N-methyl-morpholine and diisopropylamine, preferably at a temperature of −70° C. to 200° C., into at least one compound of the general formula I,
wherein the meaning of R¹, R², R³, R⁴, R⁵, R⁶, R⁸, R⁹, T, U, V, W and n is as specified above and R⁷ represents H and R¹⁰ represents —C(═O)—NH—R¹⁷ or —C(═S)—NH—R¹⁷, and this is optionally purified and/or isolated.

The present invention additionally relates to a method for producing compounds corresponding to the foregoing formula I, said method comprising reacting a compound corresponding to formula II

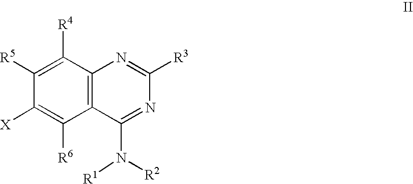

wherein R¹, R², R³, R⁴, R⁵ and R⁶ have the meanings given above and X represents a leaving group, preferably a halogen residue or a sulfonic ester, particularly preferably leaving group selected from the group consisting of chlorine, bromine, iodine, triflate, mesylate and tosylate, with a compound corresponding to formula VI

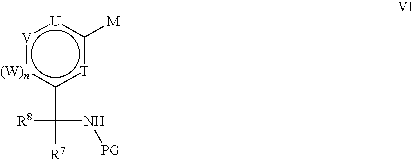

wherein T, U, V, W, n, R⁷ and R⁸ have the meanings given above, M represents —BF₃K, —B(OH)₂ or —B(OR^{A})₂, wherein R^{A} represents alkyl or two residues R^{A} together with the —O—B—O— group linking them form a heterocycloalkyl residue, preferably together with the —O—B—O— group linking them form a 1,3,2-dioxaborolan-2-yl residue, and PG represents a protecting group, preferably for a protecting group selected from the group consisting of tert-butyloxy-carbonyl, benzyl, benzyloxycarbonyl and 9-fluoroenylmethyloxycarbonyl, is reacted in at least one reaction medium, preferably in at least one reaction medium selected from the group consisting of methanol, ethyl acetate, ethanol, isopropanol, diethyl ether, dimethoxyethane, dioxane, tetrahydrofuran, dimethylformamide, acetonitrile, dimethyl sulfoxide, toluene, N-methyl-pyrrolidine and water or corresponding mixtures, possibly in the presence of at least one base, preferably in the presence of at least one base selected from the group consisting of potassium carbonate, sodium carbonate, potassium phosphate, sodium hydrogenphosphate, cesium carbonate, triethylamine, [1,4]-diazabicyclo-[2,2,2]-octane, diisopropylamine, diisopropylethylamine and N-methylmorpholine, in the presence of at least one catalyst, which can be polymer-bonded, preferably in the presence of at least one catalyst, which can be polymer-bonded, selected from the group consisting of palladium(II)acetate, tris(dibenzylideneacetone)dipalladium, palladium(0)bis-dibenzylideneacetone), tetrakis(triphenylphosphine)palladium(0), (1,1′-bis(diphenyl-phosphino)ferrocene)-dichloropalladium(II), bis(acetonitrile)dichloropalladium(II), palladium(II)chloride, dichlorobis(triphenylphosphine)palladium(II), dichloro(tricyclohexylphosphine)palladium(II), bis(acetato)bis(triphenylphosphine)palladium(II), bistriphenylphosphine palladium(II)dichloride, bistriphenylphosphine palladium(II)acetate and iron(III)chloride, possibly in the presence of at least one ligand, which can be polymer-bonded, preferably in the presence of at least one ligand, which can be polymer-bonded, selected from the group consisting of 2-dicyclohexylphosphino-2′,6′-dimethoxybiphenyl (S-phos), 2-dicyclohexylphosphino-2′,4′,6′-triisopropylbiphenyl (X-phos), tricyclohexylphosphine, tricyclohexylphosphine tetrafluoroborate, tri-tert-butylphosphine tetrafluoroborate, triphenylphosphine and imidazolium salts, preferably at a temperature of −70° C. to 300° C., to yield a compound corresponding to formula VII

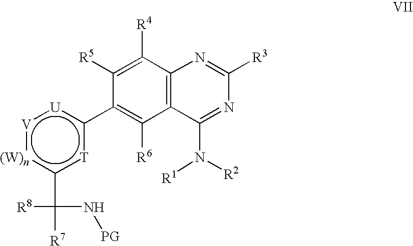

wherein R¹, R², R³, R⁴, R⁵, R⁶, R⁷, R⁸, T, U, V, W, n and PG have the meanings given above, and this is optionally purified and/or isolated; and
if PG represents a tert-butyloxycarbonyl or 9-fluoroenylmethyloxy carbonyl group, reacting a compound of formula VII in a reaction medium, preferably in a reaction medium selected from the group consisting of ethyl acetate, diethyl ether, dioxane, dichloromethane, methanol and ethanol or corresponding mixtures, in the presence of an acid, preferably in the presence of an acid selected from the group consisting of hydrochloric acid and trifluoroacetic acid, preferably at a temperature of between −70° C. to 100° C., or if PG represents a benzyl group or benzyloxycarbonyl group, reacting a compound of formula VII in a reaction medium, preferably in a reaction medium selected from the group consisting of ethyl acetate, diethyl ether, dioxane, dichloromethane, methanol and ethanol or mixtures thereof, in the presence of hydrogen and in the presence of at least one catalyst, preferably in the presence of palladium on carbon, preferably at a temperature of between −70° C. to 200° C., to yield a compound corresponding to formula I or salt thereof

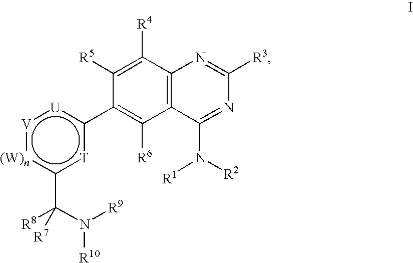

wherein the meaning of R¹, R², R³, R⁴, R⁵, R⁶, R⁷, R⁸, R¹⁰, T, U, V, W and n is as specified above and R⁹ and R¹⁰ respectively represent H,
and optionally purifying or isolating the compound of formula I; and
optionally reacting a compound of formula I, wherein R¹, R², R³, R⁴, R⁵, R⁶, R⁷, R⁸, T, U, V, W and n have the meanings given above and R⁹ and R¹⁰ each represent H, with a compound corresponding to formula Z-S(═O)₂—R²¹, wherein R²¹ has the meaning given above and Z represents a leaving group, preferably a halogen residue, particularly preferably a chlorine atom, optionally in a reaction medium, preferably in a reaction medium selected from the group consisting of tetrahydrofuran, dioxane, dichloromethane, diethyl ether, toluene, acetonitrile and dimethylformamide, or corresponding mixtures, optionally in the presence of a base, preferably in the presence of a base selected from the group consisting of triethylamine, pyridine, diisopropylethylamine, dimethylaminopyridine, N-methyl-morpholine and diisopropylamine, preferably at a temperature of −70° C. to 200° C., to yield a compound corresponding to formula I, wherein R¹, R², R³, R⁴, R⁵, R⁶, R⁷, R⁸, R⁹, T, U, V, W and n have the meanings given above and R¹⁰ represents —S(═O)₂—R²¹,
and optionally purifying or isolating the compound of formula I; or
optionally reacting a compound of formula I, wherein R¹, R², R³, R⁴, R⁵, R⁶, R⁷, R⁸, T, U, V, W and n have the meanings given above and R⁹ and R¹⁰ each represent H, with a compound corresponding to the formula Z-C(═O)—R¹⁵, wherein R¹⁵ has the meaning given above, and Z represents a leaving group, preferably a halogen residue, particularly preferably a chlorine atom, optionally in a reaction medium, preferably in a reaction medium selected from the group consisting of tetrahydrofuran, dioxane, dichloromethane, diethyl ether, toluene, acetonitrile and dimethylformamide, or mixtures thereof, optionally in the presence of a base, preferably in the presence of a base selected from the group consisting of triethylamine, pyridine, dimethylaminopyridine, diisopropylethylamine, N-methyl-morpholine and diisopropylamine, preferably at a temperature of −70° C. to 200° C., to yield a compound corresponding to formula I, wherein R¹, R², R³, R⁴, R⁵, R⁶, R⁷, R⁸, R⁹, T, U, V, W and n have the meanings given above, and R¹⁰ represents —C(═O)—R¹⁵,
and optionally purifying or isolating the compound of formula I; or
optionally reacting a compound corresponding to formula I, wherein R¹, R², R³, R⁴, R⁵, R⁶, R⁷, R⁸, T, U, V, W and n have the meanings given above, and R⁹ and R¹⁰ each represent H, with a compound corresponding to the formula OH—C(═O)—R¹⁵, wherein R¹⁵ has the meaning given above, optionally in a reaction medium, preferably in a reaction medium selected from the group consisting of tetrahydrofuran, dioxane, dichloromethane, diethyl ether, toluene, acetonitrile and dimethylformamide, or mixtures thereof, optionally in the presence of a base, preferably in the presence of a base selected from the group consisting of triethylamine, pyridine, diisopropylethylamine, dimethylaminopyridine, N-methyl-morpholine and diisopropylamine, in the presence of a coupling reagent, preferably in the presence of a coupling reagent selected from the group consisting of 1-benzotriazolyloxy-tris-(dimethylamino)-phosphonium hexafluorophosphate (BOP), dicyclohexylcarbodiimide (DCC), N′-(3-dimethylaminopropyl)-N-ethylcarbodiimide (EDCI), diisopropylcarbodiimide, 1,1′-carbonyl-diimidazole (CDI), N-[(dimethylamino)-1H-1,2,3-triazolo[4,5-b]pyridino-1-ylmethylene]-N-methylmethanaminium hexafluorophosphate N-oxide (HATU), O-(benzotriazol-1-yl)-N,N,N′,N′-tetramethyluroniome hexafluorophosphate (HBTU), O-(benzotriazol-1-yl)-N,N,N′,N′-tetramethyluronium-tetrafluoroborate (TBTU), pentafluorophenyl trifluoroacetate, 1-hydroxy-1H-benzotriazole (HOBT) and 1-hydroxy-7-azabenzotriazole (HOAt), which can respectively be polymer-bonded,
preferably at a temperature of −70° C. to 200° C., to yield a compound of formula I, wherein R¹, R², R³, R⁴, R⁵, R⁶, R⁷, R⁸, R⁹, T, U, V, W and n have the meanings given above, and R¹⁰ represents —C(═O)—R¹⁵,
and optionally purifying or isolating the compound of formula I; or
optionally reacting a compound corresponding to formula I, wherein R¹, R², R³, R⁴, R⁵, R⁶, R⁷, R⁸, T, U, V, W and n have the meanings given above, and R⁹ and R¹⁰ each represent H, with a compound corresponding to the formula R¹⁷—N═C═O or a compound corresponding to the formula R¹⁷—N═C═S, wherein R¹⁷ has the meaning given above, optionally in a reaction medium, preferably in a reaction medium selected from the group consisting of tetrahydrofuran, dioxane, dichloromethane, diethyl ether, toluene, acetonitrile and dimethylformamide, or mixtures thereof, optionally in the presence of a base, preferably in the presence of a base selected from the group consisting of triethylamine, pyridine, diisopropylethylamine, dimethylaminopyridine, N-methyl-morpholine and diisopropylamine, preferably at a temperature of −70° C. to 200° C., to yield a compound of formula I, wherein R¹, R², R³, R⁴, R⁵, R⁶, R⁷, R⁸, R⁹, T, U, V, W and n have the meanings given above, and R¹⁰ represents —C(═O)—NH—R¹⁷ or —C(═S)—NH—R¹⁷,
and optionally purifying or isolating the compound of formula I; or
optionally reacting a compound corresponding to formula I, wherein R¹, R², R³, R⁴, R⁵, R⁶, R⁷, R⁸, T, U, V, W and n have the meanings given above, and R⁹ and R¹⁰ each represent H, with a compound, which has at least two substituents independently selected from the group consisting of bromine, chlorine, —S(═O)₂—Cl, —S(═O)₂—Br, —C(═O)—Cl and —C(═O)—Br, optionally in a reaction medium, preferably in a reaction medium selected from the group consisting of tetrahydrofuran, dioxane, dichloromethane, diethyl ether, toluene, acetonitrile and dimethylformamide, or mixtures thereof, optionally in the presence of a base, preferably in the presence of a base selected from the group consisting of triethylamine, pyridine, diisopropylethylamine, dimethylaminopyridine, N-methyl-morpholine and diisopropylamine, preferably at a temperature of −70° C. to 200° C., to yield a compound corresponding to formula I, wherein R¹, R², R³, R⁴, R⁵, R⁶, R⁷, R⁸, T, U, V, W and n have the meanings given above, and R⁹ and R¹⁰ together with the nitrogen atom to which they are bound form a heterocycloalkyl or a heterocycloalkenyl residue,
and optionally purifying or isolating the compound of formula I.

The present invention additionally relates to a method for producing compounds corresponding to the foregoing formula I, said method comprising reacting a compound corresponding to formula II

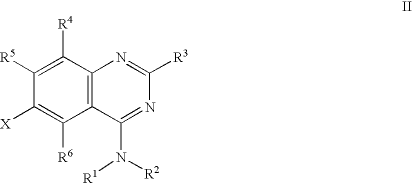

wherein the meaning of R¹, R², R³, R⁴, R⁵ and R⁶ is as specified above and X represents a leaving group, preferably a halogen residue or a sulfonic ester, particularly preferably a leaving group selected from the group consisting of chlorine, bromine, iodine, triflate, mesylate and tosylate,
with a compound corresponding to formula III

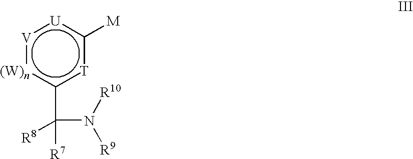

wherein T, U, V, W, n, R⁷, R⁸, R⁹ and R¹⁰ have the meanings given above, and M represents —Mg—Y or —ZnY, wherein Y represents a halogen residue or a sulfonic ester, preferably chlorine, bromine, iodine, mesylate or triflate, or M represents —BF₃K, —B(OH)₂ or —B(OR^{A})₂, wherein R^{A} represents alkyl or two residues R^{A} together with the —O—B—O— group linking them form a heterocycloalkyl residue, preferably together with the —O—B—O— group linking them form a 1,3,2-dioxaborolan-2-yl residue, optionally in a reaction medium, preferably in a reaction medium selected from the group consisting of methanol, ethyl acetate, ethanol, isopropanol, diethyl ether, dioxane, tetrahydrofuran, dimethylformamide, acetonitrile, dimethoxyethane, dimethyl sulfoxide, toluene, N-methyl-pyrrolidine and water or corresponding mixtures, optionally in the presence of a base, preferably in the presence of a base selected from the group consisting of potassium carbonate, sodium carbonate, potassium phosphate, sodium hydrogenphosphate, cesium carbonate, triethylamine, [1,4]-diazabicyclo-[2,2,2]-octane, diisopropylamine, diisopropylethylamine and N-methyl-morpholine, in the presence of at least one catalyst, which can be polymer-bonded, preferably in the presence of at least one catalyst, which can be polymer-bonded, selected from the group consisting of palladium(II)acetate, tris(dibenzylideneacetone)dipalladium, palladium(0)bis-dibenzylideneacetone), tetrakis(triphenylphosphine)palladium(0), (1,1′-bis(diphenyl-phosphino)ferrocene)-dichloropalladium(II), bis(acetonitrile)dichloro-palladium(II), palladium(II)chloride, dichlorobis(triphenylphosphine)palladium(II), dichloro(tricyclohexylphosphine)palladium(II), bis(acetato)bis(triphenylphosphine)palladium(II), bistriphenylphosphine palladium(II)dichloride, bistriphenylphosphine palladium(II)acetate and iron(III)chloride, optionally in the presence of a ligand, which can be polymer-bonded, preferably in the presence of a ligand, which can be polymer-bonded, selected from the group consisting of 2-dicyclohexylphosphino-2′,6′-dimethoxybiphenyl (S-phos), 2-dicyclohexylphosphino-2′,4′,6′-triisopropylbiphenyl (X-phos), tricyclohexylphosphine, tricyclohexylphosphine tetrafluoroborate, tri-tert-butylphosphine tetrafluoro-borate, triphenylphosphine and imidazolium salts, preferably at a temperature of −70° C. to 300° C., to yield a compound corresponding to formula I

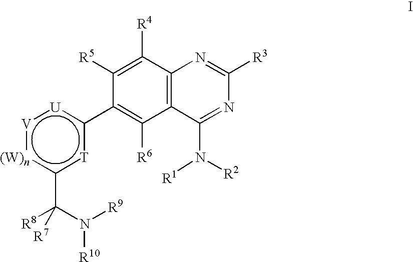

wherein R¹, R², R³, R⁴, R⁵, R⁶, R⁷, R⁸, R⁹, R¹⁰, T, U, V, W and n have the meanings given above,
and optionally purifying or isolating the compound of formula I.

It is particularly preferred if the reaction of compounds of the general formula II with compounds of the general formula III or VI, wherein M represents —B(OH)₂ or —B(OR^{A})₂, to compounds of the general formula IV or VII or I respectively occurs in toluene or dioxane as reaction medium with the addition of ethanol and/or water, in the presence of at least one base selected from the group consisting of potassium carbonate, sodium carbonate and cesium carbonate and in the presence of tetrakis-triphenylphosphine palladium(0) at a temperature of between 70° C. and 120° C.

It is particularly preferred if compounds of the general formula IV are converted to compounds of the general formula V in methanol as reaction medium with sodium borohydride as reducing agent at a temperature of between 0° C. and 30° C.

It is particularly preferred if the reaction of compounds corresponding to formula V with compounds of formula HNR⁹R¹⁰ to compounds of the general formula I, wherein the meaning of R¹, R², R³, R⁴, R⁵, R⁶, R⁷, R⁸, R⁹, R¹⁰, T, U, V, W and n is as specified above, and R⁷ represents H, occurs in tetrahydrofuran, in the presence of triphenylphosphine and diisopropylazodicarboxylate at a temperature between 20° C. and 30° C.

It is particularly preferred if compounds of the general formula IV are reacted with compounds corresponding to the formula H₂NR⁹ to obtain compounds of formula I, wherein R¹, R², R³, R⁴, R⁵, R⁶, R⁷, R⁸, R⁹, T, U, V, W and n have the meanings given above, and R⁷ and R¹⁰ each represent H, occurs in tetrahydrofuran as reaction medium in the presence of sodium triacetoxyborohydride at a temperature of between 20° C. and 30° C.

It is particularly preferred if compounds corresponding to formula I are reacted with compounds corresponding to the formula Z-S(═O)₂—R²¹ in tetrahydrofuran or dichloro-methane as reaction medium in the presence of triethylamine or diisopropylethylamine at a temperature of between −70° C. and 30° C.

It is particularly preferred if compounds corresponding to formula I are reacted with compounds corresponding to formula Z-C(═O)—R¹⁵ in tetrahydrofuran or dichloromethane as reaction medium in the presence of triethylamine or diisopropylethylamine at a temperature of between −70° C. and 30° C.

It is particularly preferred if compounds of formula I are reacted with compounds of the formula HO—C(═O)₂—R¹⁵ in tetrahydrofuran or dichloromethane as reaction medium in the presence of triethylamine or diisopropylethylamine and in the presence of pentafluorophenyl trifluoroacetate at a temperature between 0° C. and 30° C.

It is particularly preferred if compounds of formula I are reacted with compounds of the formula R¹⁷—N═C═O or R¹⁷—N═C═S in toluene as a reaction medium at a temperature between 100° C. and 120° C.

The synthesis of substituted 4-amino-quinazoline compounds corresponding to formula II, wherein R³ represents hydrogen, an alkyl residue or an aryl or heteroaryl residue, takes place as shown in Diagram 1 starting from the corresponding substituted anthranilic acids of formula VIII following procedures known from the technical literature, for example, from D. J. Connolly et al., Tetrahedron, 61:10153-10202 (2005).

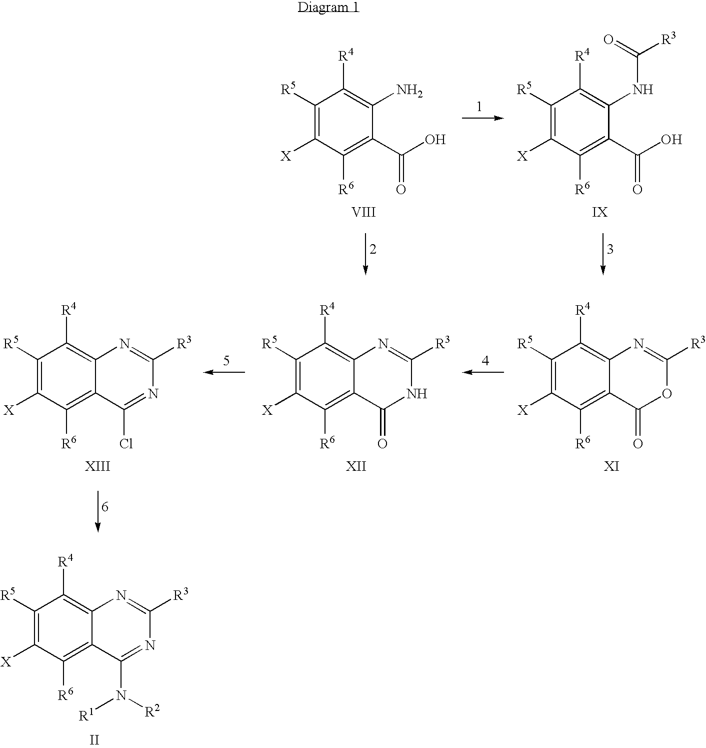

The synthesis of substituted 4-amino-quinazoline compounds of the general formula II, wherein R³ represents a halogen residue, occurs as shown in Diagram 2 working from the corresponding substituted anthranilic acids of the general formula VIII according to the directions known from specialist literature as described in D. J. Connolly et al., Tetrahedron, 2005, 61, 10153-10202. The corresponding parts of the publication apply herewith as part of the disclosure.

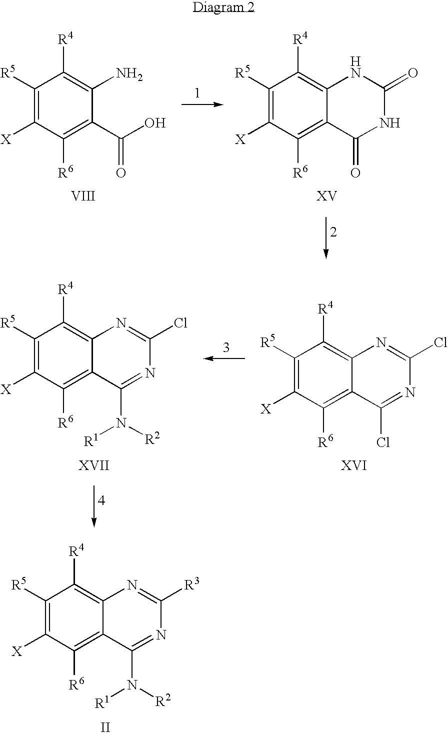

The compounds of the above-specified formulae II, III, VIII, HNR⁹R¹⁰, H₂NR⁹, Z-S(═O)₂—R²¹, Z-C(═O)—R¹⁵ and HO—C(═O)—R¹⁵ are respectively commercially available and/or can be manufactured by conventional processes known to persons skilled in the art.

The present invention additionally relates to a method for producing compounds corresponding to the foregoing formula I, according to which a compound of formula XIII

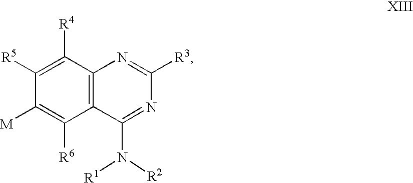

wherein R¹, R², R³, R⁴, R⁵ and R⁶ have the meanings specified above, and M represents —MgY or —ZnY, wherein Y represents a halogen residue or a sulfonic ester, preferably chlorine, bromine, iodine, mesylate or triflate, or M represents —BF₃K, —B(OH)₂ or —B(OR^{A})₂, wherein R^{A} represents alkyl or two residues R^{A} together with the —O—B—O— group linking them form a heterocycloalkyl residue, preferably a 1,3,2-dioxaborolan-2-yl residue, is reacted with a compound corresponding to formula IX

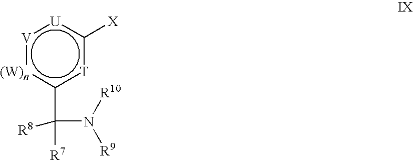

wherein T, U, V, W, n, R⁷, R⁸, R⁹ and R¹⁰ have the meanings specified above, and X represents a leaving group, preferably a halogen residue or a sulfonic ester, particularly preferably a leaving group selected from the group consisting of chlorine, bromine, iodine, triflate, mesylate and tosylate, optionally in a reaction medium, preferably in a reaction medium selected from the group consisting of methanol, ethyl acetate, ethanol, isopropanol, diethyl ether, dioxane, tetrahydrofuran, dimethylformamide, acetonitrile, dimethoxyethane, dimethyl sulfoxide, toluene, N-methyl-pyrrolidine and water or a mixture thereof, optionally in the presence of a base, preferably in the presence of a base selected from the group consisting of potassium carbonate, sodium carbonate, potassium phosphate, sodium hydrogenphosphate, cesium carbonate, triethylamine, [1,4]-diazabicyclo-[2,2,2]-octane, diisopropylamine, diisopropylethylamine and N-methylmorpholine, in the presence of a catalyst, which optionally may be polymer-bonded, preferably in the presence of a catalyst which can be polymer-bonded, selected from the group consisting of palladium(II)acetate, tris(dibenzylideneacetone)dipalladium, palladium(0)bis-dibenzylideneacetone), tetrakis(triphenylphosphine)palladium(0), (1,1′-bis(diphenyl-phosphino)ferrocene)-dichloropalladium(II), bis(acetonitrile)dichloropalladium(II), palladium(II)chloride, dichlorobis(triphenylphosphine)palladium(II), dichloro(tricyclohexylphosphine)palladium(II), bis(acetato)bis(triphenylphosphine)palladium(II), bistriphenylphosphine palladium(II)dichloride, bistriphenylphosphine palladium(II)acetate and iron(III)chloride, optionally in the presence of a ligand, which optionally may be polymer-bonded, preferably in the presence of a ligand, which can be polymer-bonded, selected from the group consisting of 2-dicyclohexylphosphino-2′,6′-dimethoxybiphenyl (S-phos), 2-dicyclohexylphosphino-2′,4′,6′-triisopropylbiphenyl (X-phos), tricyclohexylphosphine, tricyclohexylphosphine tetrafluoroborate, tri-tert-butylphosphine tetrafluoroborate, triphenylphosphine and imidazolium salts, preferably at a temperature of −70° C. to 300° C., to yield a compound corresponding to formula I

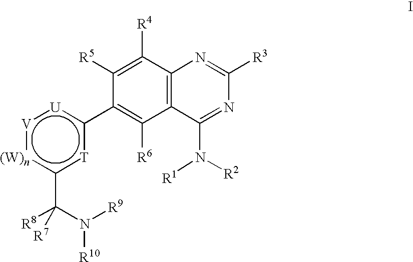

wherein R¹, R², R³, R⁴, R⁵, R⁶, R⁷, R⁸, R⁹, R¹⁰, T, U, V, W and n have the meanings specified above, and optionally purifying or isolating the compound of formula I.

It is particularly preferred if the reaction of a compound of formula II with a compound of formula XIII, wherein M represents —B(OH)₂ or —B(OR^{A})₂, to yield a compound of formula IX occurs in a 1,2-dimethoxyethane, toluene or dioxane reaction medium with the addition of ethanol and/or water, in the presence of a base selected from the group consisting of potassium carbonate, sodium carbonate and cesium carbonate and in the presence of tetrakis-triphenylphosphine palladium(0) at a temperature between 70° C. and 120° C.

The reactions described above can be carried out under conventional conditions known to persons skilled in the art, e.g. with respect to pressure or sequence of addition of the components. If necessary, the optimum procedure in accordance with the respective conditions can be determined by persons skilled in the art by simple preliminary tests.

If desired and/or if necessary, the intermediate and end products obtained after the above-described reactions can be purified and/or isolated by conventional methods known to persons skilled in the art. Suitable purification processes include, for example, extraction processes and chromatographic processes such as column chromatography or preparative chromatography.

All the process steps described above as well as the respective purification and/or isolation of intermediate and end products can be carried out partially or completely under an inert gas atmosphere, preferably under a nitrogen atmosphere.

Where the substituted 4-amino-quinazoline compounds of the invention corresponding to the foregoing formulas I, Ia, Ib or Ic (referred to hereinafter as substituted 4-amino-quinazoline compounds of formula I) have been obtained after their production in the form of a mixture of their stereoisomers, preferably in the form of their racemates or other mixtures of their different enantiomers and/or diastereomers, these can be separated and isolated, if desired, by conventional processes known to persons skilled in the art. Examples include chromatographic separation processes, in particular liquid chromatography processes under normal pressure or under elevated pressure, preferably MPLC and HPLC processes, as well as fractional crystallisation processes. In this case, individual enantiomers can be separated from one another in particular e.g. by HPLC on a chiral phase or by crystallisation with chiral acids, for instance (+) tartaric acid, (−) tartaric acid, or (+) 10-camphorsulfonic acid, formed diasteriomeric salts.

The substituted 4-amino-quinazoline compounds according to the invention corresponding to the foregoing formula I, as well as possible corresponding stereoisomers, can be obtained in the form of corresponding salts by conventional processes known to persons skilled in the art, preferably in the form of corresponding physiologically acceptable salts, particularly in the form of corresponding hydrochlorides, and the pharmaceutical composition of the invention may contain one or more salts of one or more compounds.

The respective salts of the substituted 4-amino-quinazoline compounds of the above-specified general formula I according to the invention as well as corresponding stereoisomers can be obtained, for example, by conversion with one or more inorganic acids and/or one or more organic acids. Suitable acids can preferably be selected from the group consisting of perchloric acid, hydrochloric acid, hydrobromic acid, sulfuric acid, methane-sulfonic acid, formic acid, acetic acid, oxalic acid, succinic acid, tartaric acid, mandelic acid, fumaric acid, lactic acid, citric acid, glutamic acid, saccharinic acid, cyclohexane sulfamic acid, aspartame, monomethyl sebacic acid, 5-oxo-proline, hexane-1-sulfonic acid, nicotinic acid, 2-aminobenzoic acid, 3-aminobenzoic acid or 4-aminobenzoic acid, 2,4,6-trimethylbenzoic acid, α-liponic acid, acetylglycine, hippuric acid, phosphoric acid, maleic acid, malonic acid and aspartic acid.

The substituted 4-amino-quinazoline compounds corresponding to the foregoing formula I according to the invention, as well as possible corresponding stereoisomers and their respective physiologically acceptable salts, can also be obtained in the form of solvates, in particular in the form of hydrates, by conventional processes known to persons skilled in the art.

It has surprisingly been found that the substituted 4-amino-quinazoline compounds corresponding to the foregoing formula I according to the invention are suitable for mGluR5 receptor regulation and can therefore be used in particular as pharmaceutical adjuvants in pharmaceutical compositions for the inhibition and/or treatment of disorders or diseases associated with these receptors or processes.

The substituted 4-amino-quinazoline compounds corresponding to the foregoing formula I according to the invention and possible corresponding stereoisomers and also the corresponding salts and solvates appear to be toxicologically safe and are therefore suitable as pharmaceutical adjuvants in pharmaceutical compositions.

Accordingly, the present invention additionally relates to a pharmaceutical composition containing at least one substituted 4-amino-quinazoline compound corresponding to the foregoing formula I according to the invention, optionally in the form of one of its pure stereoisomers, in particular enantiomers or diastereomers, its racemates, or in the form of a mixture of stereoisomers, in particular the enantiomers and/or diastereomers, in any mixture ratio, or respectively in the form of a corresponding salt or respectively in the form of a corresponding solvate, as well as optionally one or more pharmaceutically acceptable adjuvants.

The compounds and pharmaceutical compositions of the invention are suitable for mGluR5 receptor regulation, in particular for inhibition of the mGluR5 receptor. The compounds and pharmaceutical compositions according to the invention therefore are advantageously suitable for the inhibition and/or treatment of disorders and/or diseases that are at least partially mediated by mGluR5 receptors. The compounds and pharmaceutical compositions according to the invention are particularly suitable for the treatment and/or inhibition of pain, preferably pain selected from the group consisting of acute pain, chronic pain, neuropathic pain and visceral pain; migraine; depression; neurodegenerative diseases, preferably selected from the group consisting of multiple sclerosis, Alzheimer's disease, Parkinson's disease and Huntington's disease; cognitive diseases, preferably cognitive difficiencies, particularly preferably in relation to attention deficit disorder (ADS); psychiatric disorders, preferably anxiety conditions and panic attacks; epilepsy; coughing; urinary incontinence; diarrhoea; pruritus; schizophrenia; cerebral ischemia; muscle spasms; cramps; lung diseases, preferably selected from the group consisting of asthma and pseudo-croup; regurgitation (vomiting); stroke; dyskinesia; retinopathy; lethargy; laryngitis; eating disorders, preferably selected from the group consisting of bulimia, cachexia, anorexia and obesity; alcohol dependence; medication dependence; drug dependence, preferably nicotine and/or cocaine dependence; alcohol abuse; medication abuse; drug abuse; preferably nicotine and/or cocaine abuse; withdrawal symptoms in the case of alcohol, medication and/or drug (in particular nicotine and/or cocaine) dependence; tolerance development with respect to medications, preferably with respect to natural or synthetic opioids; gastro-esophageal reflux syndrome; gastro-esophageal reflux disease; irritable bowel syndrome; for diuresis; for antinatriuresis; for influencing the cardiovascular system; for increasing vigilance; for increasing libido; for modulating movement activity or for local anaesthesia. It is particularly preferred that the compounds and pharmaceutical compositions according to the invention is suitable for the inhibition of pain, preferably of pain selected from the group consisting of acute pain, chronic pain, neuropathic pain and visceral pain; psychiatric disorders, preferably anxiety conditions and panic attacks; cognitive diseases, preferably cognitive difficiencies, particularly preferred in relation to attention deficit disorder (ADS); gastro-esophageal reflux syndrome, gastro-esophageal reflux disease and irritable bowel syndrome. It is even more preferred that the compounds and pharmaceutical compositions according to the invention are suitable for the inhibition and/or treatment of pain, preferably of pain selected from the group consisting of acute pain, chronic pain, neuropathic pain and visceral pain. It is likewise even more preferred that the compounds and pharmaceutical compositions according to the invention are suitable for the inhibition and/or treatment of psychiatric disorders, preferably anxiety conditions and panic attacks.

The present invention additionally relates to the use of a substituted 4-amino-quinazoline compound corresponding to the foregoing formula I according to the invention, optionally in the form of one of its pure stereoisomers, in particular enantiomers or diastereomers, its racemates, or in the form of a mixture of stereoisomers, in particular the enantiomers and/or diastereomers, in any mixture ratio, or respectively in the form of a corresponding salt, or respectively in the form of a corresponding solvate, as well as optionally one or more pharmaceutically acceptable adjuvants for the production of a pharmaceutical composition for mGluR5 receptor regulation, preferably for inhibition of the mGluR5 receptor.

It is preferred to use the substituted 4-amino-quinazoline compounds corresponding to the foregoing formula I according to the invention, optionally in the form of one of their pure stereoisomers, in particular enantiomers or diastereomers, its racemates, or in the form of a mixture of stereoisomers, in particular the enantiomers and/or diastereomers, in any mixture ratio, or respectively in the form of a corresponding salt, or respectively in the form of a corresponding solvate, as well as optionally one or more pharmaceutically acceptable adjuvants, for the production of a pharmaceutical composition for the inhibition and/or treatment of disorders and/or diseases that are at least partially mediated by mGluR5 receptors.

It also is particularly preferred to use the substituted 4-amino-quinazoline compounds corresponding to the foregoing formula I according to the invention, optionally in the form of one of their pure stereoisomers, in particular enantiomers or diastereomers, their racemates, or in the form of a mixture of stereoisomers, in particular the enantiomers and/or diastereomers, in any mixture ratio, or respectively in the form of a corresponding salt, or respectively in the form of a corresponding solvate, as well as optionally one or more pharmaceutically acceptable adjuvants, for the production of a pharmaceutical composition for the inhibition and/or treatment of pain, especially of pain selected from the group consisting of acute pain, chronic pain, neuropathic pain and visceral pain; migraine; depression; neurodegenerative diseases, preferably selected from the group consisting of multiple sclerosis, Alzheimer's disease, Parkinson's disease and Huntington's disease; cognitive diseases, preferably cognitive difficiencies, particularly preferably in relation to attention deficit disorder (ADS); psychiatric disorders, preferably anxiety conditions and panic attacks; epilepsy; coughing; urinary incontinence; diarrhea; pruritus; schizophrenia; cerebral ischaemia; muscle spasms; cramps; lung diseases, preferably selected from the group consisting of asthma and pseudo-croup; regurgitation (vomiting); stroke; dyskinesia; retinopathy; lethargy; laryngitis; eating disorders, preferably selected from the group consisting of bulimia, cachexia, anorexia and obesity; alcohol dependence; medication dependence; drug dependence, preferably nicotine and/or cocaine dependence; alcohol abuse; medication abuse; drug abuse; preferably nicotine and/or cocaine abuse; withdrawal symptoms in the case of alcohol, medication and/or drug (in particular nicotine and/or cocaine) dependence; tolerance development with respect to medications, preferably with respect to natural or synthetic opioids; gastro-oesophageal reflux syndrome; gastro-esophageal reflux disease; irritable bowel syndrome; for diuresis; for antinatriuresis; for influencing the cardiovascular system; for increasing vigilance; for increasing libido; for modulating movement activity or for local anaesthesia.

It is most particularly preferred to use the substituted 4-amino-quinazoline compounds corresponding to the foregoing formula I according to the invention, optionally in the form of one of their respective pure stereoisomers, in particular enantiomers or diastereomers, their racemates, or in the form of a mixture of stereoisomers, in particular the enantiomers and/or diastereomers, in any mixture ratio, or respectively in the form of a corresponding salt, or respectively in the form of a corresponding solvate, as well as optionally one or more pharmaceutically acceptable adjuvants, for the production of a pharmaceutical composition for the inhibition and/or treatment of pain, especially of pain selected from the group consisting of acute pain, chronic pain, neuropathic pain and visceral pain; psychiatric disorders, preferably anxiety conditions and panic attacks; cognitive diseases, preferably cognitive difficiencies, particularly preferably in relation to attention deficit disorder (ADS); gastro-esophageal reflux syndrome, gastro-esophageal reflux disease and irritable bowel syndrome.

It is even more preferred to use the substituted 4-amino-quinazoline compounds corresponding to the foregoing formula I according to the invention, optionally in the form of one of their respective pure stereoisomers, in particular enantiomers or diastereomers, their racemates, or in the form of a mixture of stereoisomers, in particular the enantiomers and/or diastereomers, in any mixture ratio, or respectively in the form of a corresponding salt, or respectively in the form of a corresponding solvate, as well as optionally one or more pharmaceutically acceptable adjuvants, for the production of a pharmaceutical composition for the inhibition and/or treatment of pain, especially of pain selected from the group consisting of acute pain, chronic pain, neuropathic pain and visceral pain; and psychiatric disorders, preferably anxiety conditions and panic attacks.

The compounds and pharmaceutical compositions according to the invention are suitable for administration to adults and children, including small children and babies. The compounds and pharmaceutical compositions according to the invention can be provided as liquid, semi-solid or solid medicament, e.g. in the form of injection solutions, drops, juices, syrups, sprays, suspensions, tablets, patches, capsules, plasters, suppositories, ointments, creams, lotions, gels, emulsions, aerosols or in multiparticulate form, e.g. in the form of pellets or granulates, optionally pressed to form tablets, filled into capsules or suspended in a liquid, and can also be administered as such.

In addition to at least one substituted 4-amino-quinazoline compound corresponding to the foregoing formula I according to the invention, optionally in the form of its respective pure stereoisomers, in particular enantiomers or diastereomers, its racemates or in the form of mixtures of stereoisomers, in particular the enantiomers and/or diastereomers, in any mixture ratio, or optionally in the form of corresponding salts or in the form of a respective corresponding solvate, a pharmaceutical composition according to the invention usually contains further physiologically acceptable pharmaceutical adjuvants, which can preferably be selected from the group consisting of support materials, fillers, solvents, diluents, surfactants, coloring agents, preservatives, disintegrants, slip agents, lubricants, flavorings and binders. The selection of physiologically acceptable adjuvants as well as the quantities thereof to be used depends on whether the drug is to be administered orally, subcutaneously, parenterally, intravenously, intraperitoneally, intradermally, intramuscularly, intranasally, by buccal, rectal or local route, e.g. to infections on the skin, the mucous membranes and the eyes. Preparations preferably suited to oral application are those in the form of tablets, coated tablets, capsules, granulates, pellets, drops, juices and syrups, while solutions, suspensions, easily reconstituted dry preparations as well as sprays are suitable for parenteral, topical and inhalatory administration. Suitable preparations for percutaneous administration are preparations containing a substituted 4-amino-quinazoline compound corresponding to the foregoing formula I according to the invention in a depot in dissolved form or in a plaster, optionally with the addition of agents promoting skin penetration. Preparation forms that can be administered orally or percutaneously can also release the respective substituted 4-amino-quinazoline compounds of the above-specified general formula I as slow-release.

The pharmaceutical compositions according to the invention can be produced using conventional means, devices, methods and processes well known in the art, such as those described, for example, in “Remington's Pharmaceutical Sciences”, editor A. R. Gennaro, 17th edition, Mack Publishing Company, Easton, Pa., 1985, in particular in part 8, chapters 76 to 93.

The quantity of the respective substituted 4-amino-quinazoline compound corresponding to the foregoing formula I to be administered to the patient can vary and depends, for example, on the weight and age of the patient as well as on the mode of administration and the degree of severity of the disease. 0.05 to 100 mg/kg, preferably 0.05 to 10 mg/kg, body weight of the patient of at least one such compound is typically administered.

### Pharmacological Methods:

I. Method for Determining the Inhibition of the [³H]-MPEP Bond in the mGluR5 Receptor Binding Assay

Pig brain homogenate is produced by homogenizing (Polytron PT 3000, Kinematica AG, 10 000 rpm for 90 seconds) pig brain halves without medulla, cerebellum and pons in pH 8.0 buffer (20 mM Hepes, Sigma, order no. H3375+1 complete tablet, 100 ml, Roche Diagnostics, order no. 1836145) in the ratio of 1:20 (brain weight/volume) and differential centrifuging at 900×g and 40 000×g. In incubation batches of 250 μl in 96-well microtitre plates 450 μg of protein from brain homogenate with 5 nM ³[H]-MPEP (Tocris, order no. R1212) (MPEP=2-methyl-6-(3-methoxyphenyl)-ethinylpyridine) and the compounds to be examined (10 μM in test) in buffer (as above) are respectively incubated at room temperature for 60 minutes.

The batches are then filtered using a Brandel cell harvester (Brandel, Robotic 9600) on unifilter plates with fibreglass filter mats (Perkin Elmer, order no. 6005177) and then washed with buffer (as above) 3-times each with 250 μl per sample. The filter plates are then dried for 60 mins. at 55° C. Then, 30 μL of Ultima Gold™ scintillator (Packard BioScience, order no. 6013159) are added per well and after 3 hours the samples are measured on the β-counter (MicroBeta, Perkin Elmer). The non-specific binding is determined by adding 10 μM of MPEP (Tocris, order no. 1212).

IIa. Formalin Test on Rats:

The formalin test (Dubuisson, D. and Dennis, S. G., 1977, Pain, 4, 161-174) represents a model for acute as well as chronic pain. By a single formalin injection into the dorsal side of a rear paw a biphase nociceptive reaction is inducted in free-moving test animals that is detected by observation of three clearly distinguishable behavior patterns. The reaction is two-phase: phase I=immediate reaction (duration up to 10 min.; paw shaking, licking), phase 2=late reaction (after a resting phase; likewise paw shaking, licking; duration up to 60 min.). The first phase reflects a direct stimulation of the peripheral nocisensors with high spinal nociceptive input or glutamate release (acute pain phase); the second phase reflects a spinal and peripheral hypersensitisation (chronic pain phase). The chronic pain component (phase 2) was evaluated in the studies presented here.

Formalin is applied subcutaneously in a volume of 50 μl and a concentration of 5% into the dorsal side of the right rear paw of each animal. The substances to be tested are administered orally (p.o.), intravenously (i.v.) or intraperitoneally (i.p.) 30 min. before the formalin injection. The specific changes in behavior such as lifting and shaking of the paw, shifting weight of the animal as well as biting and licking reactions are observed and recorded in the observation period of 21 to 27 min. after the formalin injection. The different behaviors are combined in the so-called pain rate (PR), which represents the calculation of a mean nociception reaction on the basis of part-intervals of 3 min. The P is calculated on the basis of a numerical weighting (=factor 1, 2, 3 in each case) of the observed behaviors (corresponding to behavior score 1, 2, 3) and is calculated by the following formula: PR=[(T₀×0)+(T₁×1)+(T₂×2)+T₃×3)]/180, wherein T₀, T₁, T₂ and T₃ respectively correspond to the time in seconds, in which the animal exhibits the behaviors 0, 1, 2 or 3. The group size amounts to 10 animals (n=10).

IIb. Formalin Test on Mice

Formalin is administered subcutaneously in a volume of 20 μl and a concentration of 1% into the dorsal side of the right rear paw of each animal. The substances to be tested are applied intraperitoneally (i.p.) 15 min. before the formalin injection. The specific changes in behavior such as lifting and shaking of the paw (score 3, Dubuisson & Dennis, 1977) are observed and recorded in the observation period of 21 to 24 min. after the formalin injection. The group size amounts to 10 animals (n=10).

### III. Neuropathic Pain in Rats

Efficacy against neuropathic pain was examined using the Bennett model (chronic constriction injury; Bennett and Xie, 1988, Pain 33: 87-107).

Spraque-Dawley rats with a weight of 140-160 g are provided with four loose ligatures of the right sciatic nerve under Nembutal narcosis. On the paw innervated by the damaged nerve the animals develop a hypersensitivity, which is quantified after a recovery period of one week over about four weeks using a 4° C. cold metal plate (cold allodynia). The animals are observed on this plate for a period of 2 min. and the number of pull-away reactions of the damaged paw is measured. The substance effect is determined in relation to the initial value before substance application at four points in time (15, 30, 45, 60 min. after application) over a period of one hour and the resulting area under the curve (AUC) as well as the inhibition of the cold allodynia at the individual measuring points is expressed in percent effect to the vehicle control (AUC) or to the starting value (individual measuring points). The group size amounts to n=10. The significance of an anti-allodynic effect is determined by way of the AUC values over a paired T-test (*0.05≧p>0.01; **0.01≧p>0.001; ***p≦0.001; Armitage and Berry, 1987, Stat. Methods in Medical Research, London; Blackwell Scientific Publications).

### IV. “Elevated Plus-Maze” Model

In the “elevated plus-maze” (EPM) model compounds are tested for possible anxiolytic effects. The tests are conducted on male Sprague-Dawley rats (200-250 g) and 2 “elevated plus-mazes” (Med Associates) with electronically controlled infrared light barriers are used to determine the location of the animal in the labyrinth. Each labyrinth has 2 open and 2 closed arms and a central platform. The edges of the open arms are bordered by narrow strips. The entire labyrinth is mounted on a metal stand.

At the beginning of a 5 minute test, each animal is placed individually on the central platform with its head facing the closed arm. The following parameters are determined or calculated and evaluated: number and percent of entries into the open and closed arms as well as percentage time in the open and closed arms and on the central platform. The data are analyzed by means of a 1-factorial ANOVA (comparison of treatment groups versus vehicle group). The significance level is fixed at p<0.05. All groups have a size of N=10.

The test is also described in Hogg, S. (1996): A review of the validity and variability of the elevated plus-maze as an animal model of anxiety. Pharmacol. Biochem. Behav. 54, 21-30; and Rodgers, R. J., Cole, J. C. (1994): The elevated plus-maze: pharmacology, methodology and ethology, in Cooper, S. J., Hendrie, C. A. (eds) Ethology and Psychopharmacology, Wiley & Sons, pp 9-44.

### V. Functional Ca²⁺ Influx Assay

20,000 CHO-hmGluR5 cells/well (Euroscreen, Gosselies, Belgium) are pipetted into 96-well plates (BD Biosciences, Heidelberg, Germany, Ref. 356640, clear bottom, 96-well, poly-D-lysine) and incubated overnight in HBSS buffer (Gibco 14025-050) with the following additions: 10% FCS (GIBCO, 10270-106) and doxycycline (BD Biosciences Clontech 631311 600 ng/ml).

For the functional study the cells were charged with 2 μM of Fluo-4 and 0.01% by vol. pluronic F127 (Molecular Probes Europe BV, Leiden, The Netherlands) in HBSS buffer (Hank's buffered saline solution, Gibco Invitrogen GmbH, Karlsruhe, Germany) with probenicide (Sigma P8761, 069 mg/ml) for 30 min. at 37° C.

The cells are then washed 3 times with washing buffer (HBSS buffer, Gibco No. 14025-050), with probenicide (Sigma P8761, 0.69 mg/ml) and then taken up with the same buffer ad 100 μl. After 15 min. the plates for determining the Ca²⁺ measurements in the presence of DHPG ((S)-3,5-dihydroxyphenylglycine, Tocris Biotrend Chemikalien GmbH, Cologne, Germany, final DHPG concentration: 10 μM) and also in the presence or absence of test substances are transferred into a fluorometric imaging plate reader (FLIPR, Molecular Devices, Sunnyvale, Calif.).

The Ca²⁺-dependent fluorescence is measured before and after the addition of test substances. The quantification occurs through the measurement of the highest fluorescence intensity over time. After the fluorescence base line has been recorded for 10 sec. 50 μl of test substance solution (different test substance concentrations in HBSS buffer with 1% DMSO and 0.02% Tween 20, Sigma) are added and the fluorescence signal is measured for 6 min. 50 μl of DHPG solution ((S)-3,5-dihydroxyphenylglycine, Tocris Biotrend Chemikalien GmbH, Cologne, Germany, final DHPG concentration: 10 μM) are then added and the influx of Ca²⁺ is measured simultaneously for 60 sec. The final DMSO concentration amounts to 0.25% and the final Tween 20 content amounts to 0.005%. The data are analysed with Microsoft Excel and GraphPad Prism. The dose effect curves are calculated with non-linear regression and IC₅₀ values determined. Each data point is determined 3 times and IC₅₀ values are averaged from a minimum of 2 independent measurements. Ki values are calculated according to the following formula:

Ki=IC50/(1+(AG_{conc.}/EC50)).

AG_{conc.}=10 μM; EC50 corresponds to the DHPG concentration necessary for the half-maximum influx of Ca²⁺.

The following examples serve to illustrate the invention in further detail without limiting its scope.

### EXAMPLES

Slight variations with respect to the solvents, the equivalents of the reagents/educts, the reaction times etc. can occur in analogous syntheses. The mixture ratios of solvents, mobile solvents or for chromatographic studies are given in volume/volume or % (volume).

### ABBREVIATIONS

DBU—1,8-diazabicyclo[5.4.0]undec-8-ene
KOCN—potassium cyanate
Pd(dppf)Cl₂—bis(diphenylphosphino)ferrocene)-palladium(II)-dichloride
Pd₂(dba)₃—tris(dibenzylideneacetone)dipalladium(0)
BINAP—2,2′-bis(diphenylphosphino)-1,1′-binaphthyl
Boc—tert.-butoxycarbonyl
EDCI—N-ethyl-N′-(3-dimethylaminopropyl)-carbodiimide hydrochloride
DIPEA—diisopropylethylamine
POCl₃—phosphoroxychloride
HOBt—1-hydroxy-1H-benzotriazole

The term “equivalents” means substance amount equivalents, “RT” room temperature, “conc.” concentration, “d” days, “min” minutes, “h” hours, “M” is a specified concentration in mol/l, “MeOH” methanol, “EtOH” ethanol, “THF” tetrahydrofuran, “aq.” aqueous, “sat.” saturated, “sol.” solution, “EE” ethyl acetate, “brine” sat. aq. sodium chloride sol., “DCM” dicholoromethane, “DMF” dimethylformamide.

The chemicals and solvents used were obtained commercially from the usual suppliers (Acros, Avocado, Aldrich, Bachem, Fluka, Lancaster, Maybridge, Merck, Sigma, TCI etc.) or synthetised using conventional methods known to persons skilled in the art. As stationary phase for the column chromatography silica gel 60 (0.040-0.063 mm) from E. Merck, Darmstadt was used. The thin-film chromatographic studies were conducted with HPTLC chromatoplates, silica gel 60 F 254 from E. Merck, Darmstadt. The mixture ratios of solvents, mobile solvents or for chromatographic studies are always specified in volume/volume. The analytics occurred using mass spectrometry and/or NMR. The yields of the produced compounds were not optimized. All temperatures are uncorrected.

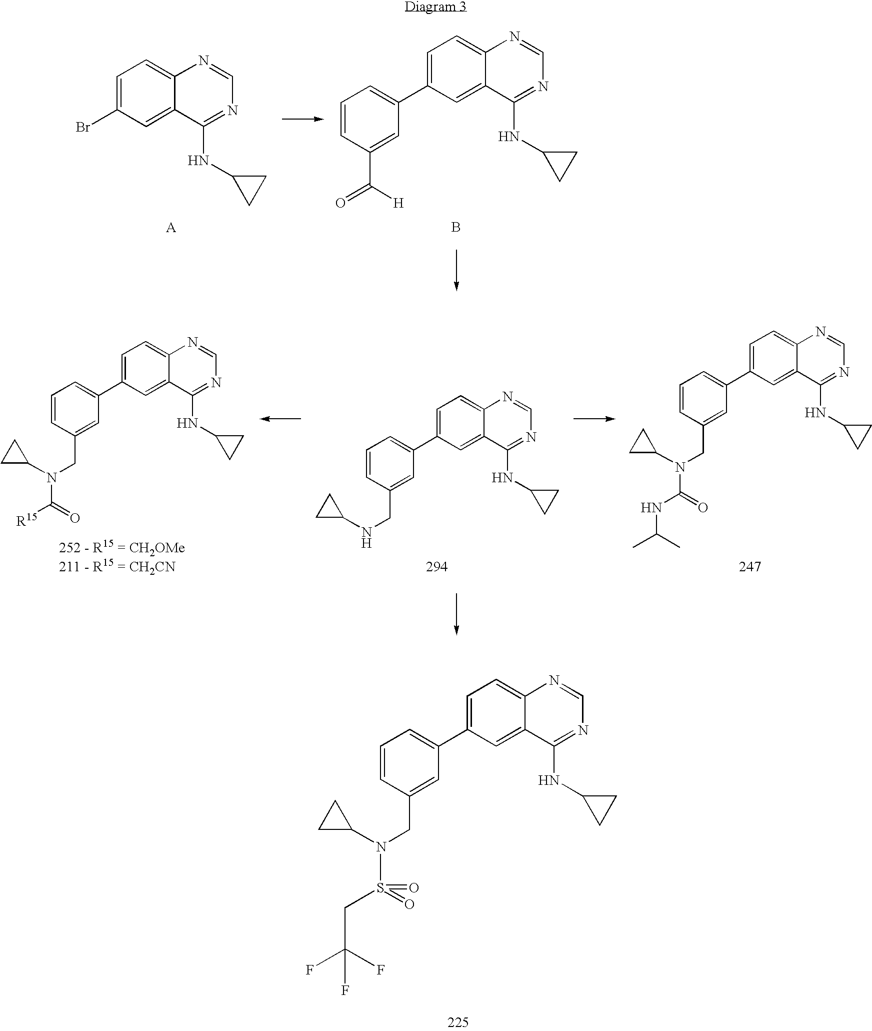

6-bromo-N-cyclopropylquinazoline-4-amine (A) was produced analogously to a direction from H. Hayashi et al., Bioorganic and Medicinal Chemistry, 2003, 11, 383.

### Synthesis of 3-(4-(cyclopropylamino)quinazolin-6-yl)benzaldehyde (B)

EtOH (20 mL) was added to a suspension of 6-bromo-N-cyclopropylquinazoline-4-amine (3.10 g, 11.74 mmol, 1 equiv.) and 3-formylphenyl boric acid (2.11 g, 14.09 mmol, 1.2 equiv.) in toluene (50 mL), followed by aq. sodium carbonate sol. (2.5 M, 20 mL) and tetrakis(triphenylphosphine)-palladium(0) (0.136 g, 0.18 mmol, 0.01 equiv.). The reaction mixture was then heated to reflux for 3 hours and evaporated to low bulk after cooling to RT. The residue was taken up in EE (150 mL) and washed with water (2×20 mL) and brine (1×20 mL), dried (MgSO₄) and the solvent removed in a vacuum. After purification by column chromatography (EE/MeOH/25% aq. ammonia sol.; 100:10:1) the desired product was obtained (2.60 g, 77%).

### Example 294

### N-cyclopropyl-6-(3-((cyclopropylamino)methyl)phenyl)quinazoline-4-amine

Cyclopropylamine (2.89 mL, 41.47 mmol, 5 equiv.) was added to a solution of 3-(4-(cyclopropylamino)quinazolin-6-yl)benzaldehyde (B) (2.40 g, 8.30 mmol, 1 equiv.) in THF (150 mL) and the mixture was then mixed with sodium cyanoborohydride (8.79 g, 41.47 mmol, 5 equiv.). The suspension was stirred for 3 days at RT, then hydrolysed by adding sat. aq. sodium hydrogencarbonate sol. (approx. 10 mL) and the solvent removed in a vacuum. The residue was taken up in EE (150 mL) and extracted with sat. aq. sodium hydrogencarbonate sol. (2×20 mL) and brine (1×20 mL), dried (MgSO₄) and the solvent removed in a vacuum. After purification by column chromatography (EE/MeOH 10:1) the desired product was obtained (294) (2.60 g, 95%).

General Direction for Converting Amines with the Carboxylic Acids of the General Formula R¹⁵—C(═O)—OH in Automated Synthesis

The corresponding carboxylic acids of the general formula R¹⁵—C(═O)—OH (1.1 equivalents), N′-(3-dimethylaminopropyl)-N-ethylcarbodiimide (EDCI) (1.2 equivalents), 1-hydroxy-1H-benzotriazole (HOBT) (1 equivalent) and diisopropylethylamine (1.5 equivalents) were mixed in dichloromethane (0.5 mL) and shaken for 30 min. The amine (50 mg respectively dissolved in 0.5 mL dichloromethane) was added to this. The reaction mixture was shaken for 16 h at RT, diluted with dichloromethane, washed with sat. aq. NH₄Cl sol. and brine, the organic phase was dried by means of sodium sulfate and the solvent removed in a vacuum. The purification of the raw products occurred in parallel via a Biotage system. The exemplary compounds 1 to 179 were obtained in this way.

### Example 252

### N-cyclopropyl-N-[3-(4-cyclopropylamino-quinazolin-6-yl)-benzyl]-2-methoxy-acetamide

Triethylamine (0.094 mL, 0.68 mmol, 1.5 equiv.) was added to a solution of N-cyclopropyl-6-(3-((cyclopropylamino)methyl)phenyl)-quinazoline-4-amine (Example 294) (0.150 g, 0.45 mmol, 1 equiv.) in DCM (5 mL) and the mixture was then cooled to −70° C. 2-methoxyacetylchloride (0.049 mL, 0.55 mmol, 1.2 equiv.) was added in drops and the mixture slowly heated to RT and stirred for 15 hours. The reaction mixture was diluted with DCM (50 mL) and extracted with sat. aq. sodium hydrogencarbonate sol. (1×10 mL) and brine (1×10 mL), dried (MgSO₄) and the solvent removed in a vacuum. After purification by column chromatography (EE/MeOH 10:1) the desired product was obtained (252) (0.150 g, 82%).

### Example 211

### 2-cyano-N-cyclopropyl-N-[3-(4-cyclopropylamino-quinazolin-6-yl)-benzyl]-acetamide

Cyanoacetic acid (0.103 mL, 1.21 mmol, 2 equiv.) and N-cyclohexylcarbodiimide-N′-methyl polystyrene resin [HL (200-400 mesh), 2% DVB] (3.4 g, 1.6 mmol/g, 3 equiv.) were added to a solution of N-cyclopropyl-6-(3-((cyclopropylamino)methyl)phenyl)-quinazoline-4-amine (Example 294) (0.200 g, 0.61 mmol, 1 equiv.) in DCM (15 mL) and the mixture then stirred at RT for 2 hours. The reaction mixture was filtered, diluted with DCM (50 mL) and extracted with sat. aq. sodium hydrogencarbonate sol. (2×5 mL), dried (MgSO₄) and the solvent removed in a vacuum to obtain the desired product (211) (0.170 g, 71%).

### Example 225

### N-cyclopropyl-N-(3-(4-(cyclopropylamino)quinazolin-6-yl)benzyl)-2,2,2-trifluoroethane sulfonamide

Triethylamine (0.125 mL, 0.91 mmol, 1.5 equiv.) was added to a solution of N-cyclopropyl-6-(3-((cyclopropylamino)methyl)phenyl)-quinazoline-4-amine (294) (0.200 g, 0.61 mmol, 1 equiv.) in DCM (7 mL) and the mixture was then cooled to −70° C. 2,2,2-trifluoroethane sulfonyl chloride (0.080 mL, 0.73 mmol, 1.2 equiv.) was added in drops and the mixture was slowly heated to RT and stirred for 15 hours. The reaction mixture was filtered, diluted with DCM (50 mL) and extracted with sat. aq. sodium hydrogencarbonate sol. (1×10 mL) and brine (1×10 mL), dried (MgSO₄) and the solvent removed in a vacuum. After purification by column chromatography (EE/MeOH 20:1) the desired product was obtained (225) (0.196 g, 69%).

### Example 246

### 1-cyclopropyl-1-[3-(4-cyclopropylamino-quinazolin-6-yl)-benzyl]-3-isopropyl-urea

Isopropylisocyanate (0.059 mL, 0.61 mmol, 1 equiv.) was added to a solution of N-cyclopropyl-6-(3-((cyclopropylamino)methyl)phenyl)-quinazoline-4-amine (294) (0.200 g, 0.61 mmol, 1 equiv.) in toluene (15 mL) and the mixture heated to reflux for 2 hours. The reaction mixture was evaporated to low bulk in vacuum and the residue was taken up in EE (50 mL) and extracted with sat. aq. sodium hydrogencarbonate sol. (2×10 mL), dried (MgSO₄) and the solvent in turn removed in a vacuum. After purification by column chromatography (EE/MeOH 10:1) the desired product was obtained (247) (0.230 g, 91%).

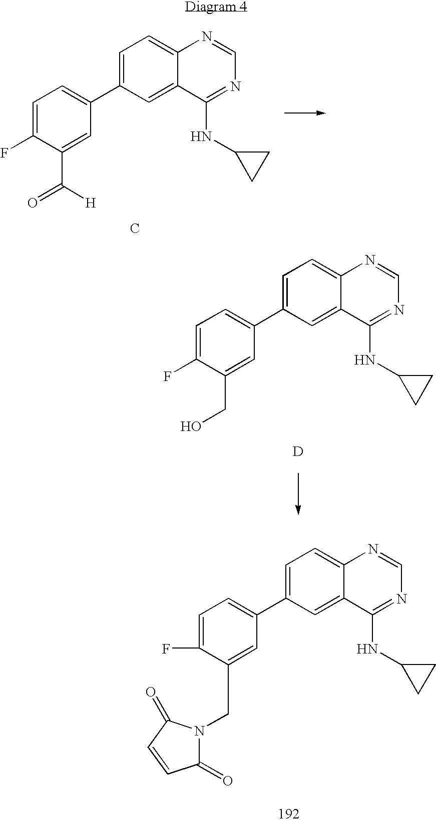

5-(4-(cyclopropylamino)quinazolin-6-yl)-2-fluorobenzaldehyde (C) was produced in the same way as compound B from the corresponding boric acid.

### Compound D:

### (5-(4-(cyclopropylamino)quinazolin-6-yl)-2-fluorophenyl)methanol

Sodium borohydride (0.322 g, 8.49 mmol, 3 equiv.) was added slowly to a suspension of 5-(4-(cyclopropylamino)quinazolin-6-yl)-2-fluorobenzaldehyde (C) (0.870 g, 2.83 mmol, 1 equiv.) in MeOH (40 mL) at 0° C. The reaction mixture was then stirred for 24 hours at RT. After hydrolysation with sat. aq. sodium hydrogencarbonate sol. (approx. 5 mL) and removal of the solvent in a vacuum, the residue was taken up in EE (100 mL) and washed with sodium hydrogencarbonate sol. (3×20 mL), dried (MgSO₄) and the organic phase evaporated to low bulk in the vacuum to obtain the desired product (D) (0.79 g, 90%).

### Example 192

### 1-(5-(4-(cyclopropylamino)quinazolin-6-yl)-2-fluorobenzyl)-1H-pyrrole-2,5-dione

Maleic acid imide (0.062 g, 0.65 mmol, 1 equiv.) was added to a solution of (5-(4-(cyclopropylamino)quinazolin-6-yl)-2-fluorophenyl)methanol (D) (0.200 g, 0.65 mmol, 1 equiv.) in THF (10 mL), followed by triphenylphosphine (0.253 g, 0.97 mmol, 1.5 equiv.) and diisopropylazodicarboxylate (0.195 mL, 0.97 mmol, 1.5 equiv.) and the mixture was then stirred for 24 hours at RT. The reaction mixture was diluted with EE (100 mL) and extracted with sat. aq. sodium hydrogencarbonate sol. (2×20 mL) and brine (20 mL), dried (MgSO₄) and the solvent removed in a vacuum. After purification by column chromatography (EE/MetOH/25% ammonia sol.; 200:10:1) the desired product was obtained (192) (0.150 g, 60%).

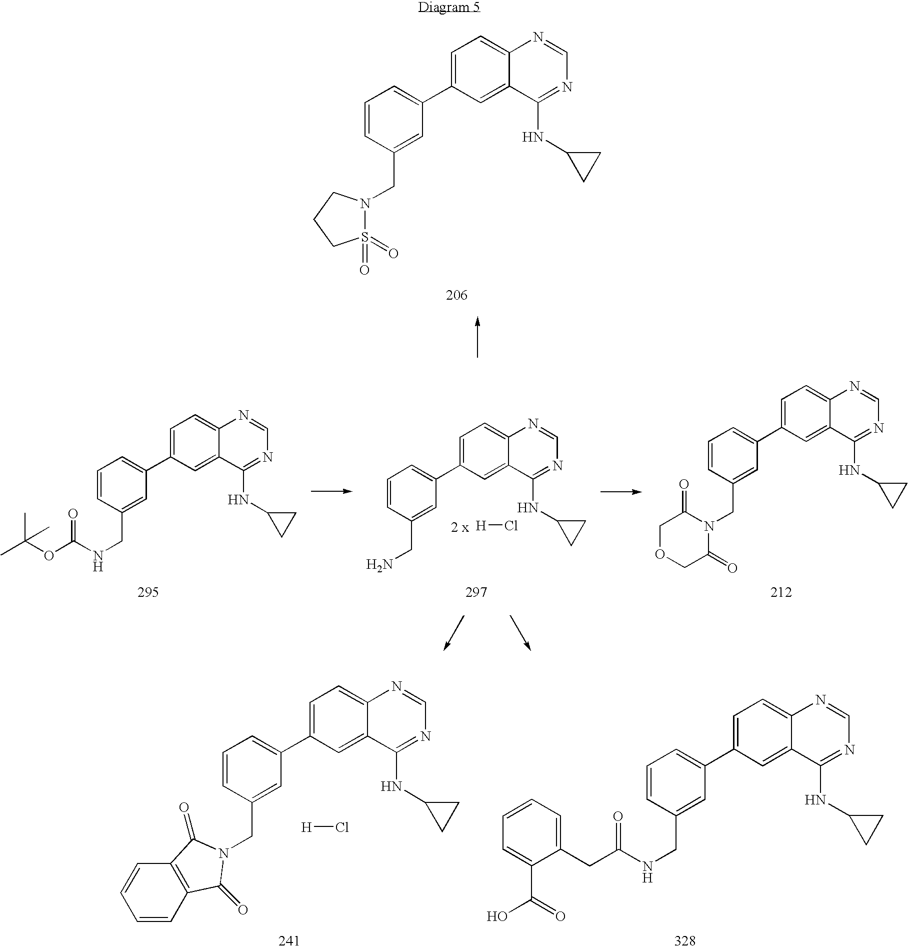

### Example 295

Tert-butyl 3-(4-(cyclopropylamino)quinazolin-6-yl)benzylcarbamate (295) was produced in the same way as compound B from the corresponding boric acid.

### Example 297

### (6-(3-(aminomethyl)phenyl)-N-cyclopropylquinazoline-4-amine dihydrochloride

Conc. aq. hydrochloric acid (37%, 9.12 mL) was slowly added in drops to a solution of tert-butyl 3-(4-(cyclopropylamino)quinazolin-6-yl)benzylcarbamate (Example 295) (4.20 g, 10.76 mmol, 1 equiv.) in MeOH (26 mL) and the mixture was then heated to reflux for 2 hours. The solvent was removed in a vacuum and the residue taken up in EE (100 mL) and diluted with diethyl ether (200 mL). The precipitate formed was filtered out and the desired product obtained (297) (3.51 g, >99%).

### Example 206

### Cyclopropyl-{6-[3-(1,1-dioxo-1λ⁶-isothiazolidin-2-ylmethyl)-phenyl]-quinazolin-4-yl}-amine hydrochloride

Potassium carbonate (0.422 g, 3.06 mmol, 5 equiv.) was added to a suspension of (6-(3-(aminomethyl)phenyl)-N-cyclopropylquinazoline-4-amine dihydrochloride (Example 297) (0.200 g, 0.61 mmol, 1 equiv.) in chloroform (20 mL), followed by 3-chloropropanesulfonic acid chloride (0.372 mL, 3.06 mmol, 5 equiv.), and the mixture was then heated to reflux for 5 hours. The reaction mixture was filtered, the solvent removed in a vacuum and the residue taken up in DMF (5 mL). Sodium hydride (0.147 g, 6.12 mmol, 10 equiv.) in DMF (10 mL) was added to this solution and the mixture stirred for 24 hours at RT. The reaction mixture was diluted with EE (80 mL) and extracted with sat. aq. sodium hydrogencarbonate sol. (2×20 mL) and brine (20 mL), dried (MgSO₄) and the solvent removed in a vacuum. After purification by column chromatography (EE/MeOH/25% ammonia sol.; 100:10:1) and hydrochloride precipitation with chlorotrimethyl silane (0.094 mL, 0.73 mmol, 1.2 equiv.) in diethyl ether (25 mL) the desired product was obtained (206) (0.100 g, 38%).

### Example 212

### 4-(3-(4-(cyclopropylamino)quinazolin-6-yl)benzyl)morpholine-3,5-dione

Triethylamine (0.363 mL, 4.59 mmol, 5 equiv.) was added to a suspension of (6-(3-(aminomethyl)phenyl)-N-cyclopropylquinazoline-4-amine dihydrochloride (Example 297) (0.300 g, 0.92 mmol, 1 equiv.) in chloroform (20 mL), followed by diglycolic acid dichloride (0.544 g, 4.59 mmol, 5 equiv.) and the mixture was then heated to reflux for 30 minutes, cooled to RT and subsequently stirred for 18 hours. The reaction mixture was evaporated to low bulk in a vacuum and the residue dissolved in DMF (5 mL). This solution was slowly added in drops at 0° C. to a suspension of sodium hydride (0.109 g, 4.59 mmol, 5 equiv.) in DMF (10 mL) and the mixture heated to reflux for 5 hours. The reaction mixture was diluted with EE (80 mL) and extracted with sat. aq. sodium hydrogencarbonate sol. (2×20 mL) and brine (20 mL), dried (MgSO₄) and the solvent removed in a vacuum. After purification by column chromatography (EE/MeOH; 20:1) the desired product was obtained (212) (0.090 g, 25%).

### Example 241

### 2-[3-(4-cyclopropylamino-quinazolin-6-yl)-benzyl]-isoindole-1,3-dione; hydrochloride

Triethylamine (0.187 mL, 1.35 mmol, 2.2 equiv.) was added to a suspension of (6-(3-(aminomethyl)phenyl)-N-cyclopropylquinazoline-4-amine dihydrochloride (Example 297) (0.200 g, 0.61 mmol, 1 equiv.) in chloroform (15 mL), followed by phthalic anhydride (0.108 g, 0.73 mmol, 1.2 equiv.) and p-toluene sulfonic acid (0.012 g, 0.07 mmol, 0.012 equiv.) and the mixture was then heated to reflux for 24 hours. The reaction mixture was evaporated to low bulk in a vacuum and the residue purified by column chromatography (EE/MeOH/25% ammonia sol.; 100:10:1). After hydrochloride precipitation with chlorotrimethyl silane (0.094 mL, 0.73 mmol, 1.2 equiv.) in diethyl ether (25 mL) the desired product was obtained (241) (0.210 g, 75%).

### Example 328

### 2-(2-(3-(4-(cyclopropylamino)quinazolin-6-yl)benzylamino)-2-oxoethyl)benzoic acid

The synthesis was conducted with ortho-carboxylphenyl acetic anhydride using the process described for Example 241. After purification by column chromatography the desired product was obtained (328) (0.080 g, 28%).

### Synthesis Diagram for Examples 186 and 187

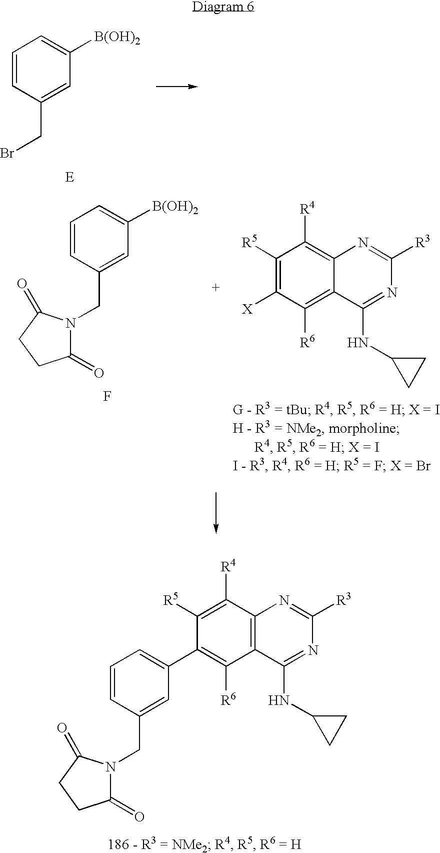

### Compound F:

### 3-((2,5-dioxopyrrolidin-1-yl)methyl)phenylboric acid

Succinimide (5.95 g, 60.00 mmol, 2 equiv.) and potassium carbonate (8.27 g, 60.00 mmol, 1 equiv.) were added to a suspension of 3-(bromomethyl)-phenylboric acid (E) (6.42 g, 30.00 mmol, 1 equiv.) in acetone (300 mL) and the mixture heated to reflux for 3 hours. After cooling to RT the solid was filtered out and the organic phase evaporated to low bulk in a vacuum. Water (200 mL) was added and the mixture adjusted to pH 7 with 5% aq. hydrochloric acid sol. The precipitated solid was filtered out and the desired product (F) (5.13 g, 73%) was obtained.

### 1-[3-(4-cyclopropylamino-2-dimethylamino-quinazolin-6-yl)-benzyl]-pyrrolidine-2,5-dione

Tetrakis(triphenylphosphine)-palladium(0) (0.100 g, 0.13 mmol, 0.09 equiv.) was added to a solution of N4-cyclopropyl-6-iodo-N2,N2-dimethylquinazoline-2,4-diamine (compound of the type H: R³=NMe₂; R⁴, R⁵, R⁶=H; X=I—Diagram 8) (0.531 g, 1.50 mmol, 1 equiv.) in dimethoxyethane (50 mL) and the mixture stirred for 10 minutes at RT. Then 3-((2,5-dioxopyrrolidin-1-yl)methyl)phenylboric acid (F) (0.454 g, 1.95 mmol, 1.3 equiv.) was added, followed by aq. sodium carbonate sol. (0.25 M, 5 mL) and the mixture was then stirred for 2 hours at 70° C. After purification by preparative thin-film chromatography (ethyl acetate), the desired product (186) (0.080 g, 30%) was obtained.

Example 292 was produced in the same way as the process described for Example 186, wherein the corresponding quinazoline-4-amine derivative can be produced according to H. Hayashi et al., Bioorganic and Medicinal Chemistry, 2003, 11, 383 or in the same way as the compound of type I (Diagram 10) and 3-(indolin-1-ylmethyl)phenylboric acid (corresponding compound of type F) was produced as described in Diagram 6.

Examples 880 and 901 were produced in the same way as the process described for Example 186, wherein 6-iodo-4-(pyrrolidin-1-yl)quinazoline and N-cyclopropyl-6-iodo-N-methylquinazoline-4-amine were produced in the same way as the compound of type I (Diagram 10). The amine N-methylcyclopropanamine hydrochloride necessary for Example 901 was produced by processes known to the person skilled in the art working from cyclopropylamine by the introduction of benzyloxycarbonyl (CBZ) protecting groups, methylation (sodium hydride, dimethylformamide, methyl iodide) and the subsequent splitting off of protecting groups.

Synthesis diagram for the production of compounds of type G, using 2-tert-butyl-N-cyclopropyl-6-iodoquinazoline-4-amine (R³=t-Bu; R⁴, R⁵, R⁶=H; X=I) as example

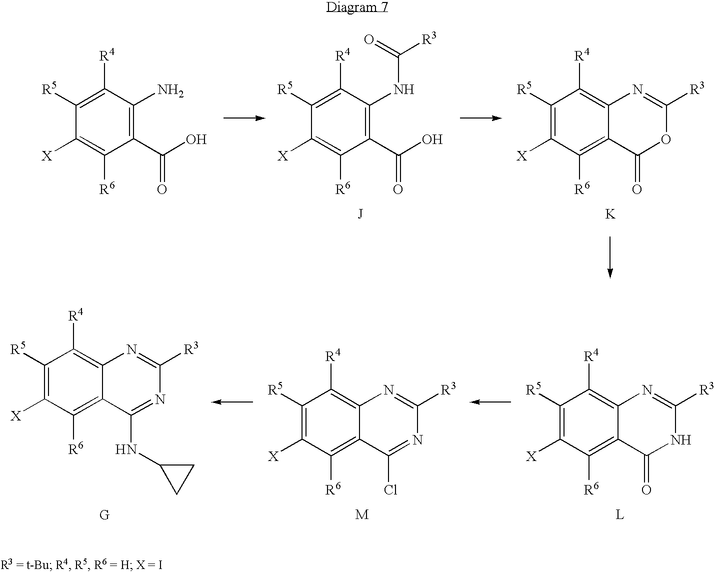

### Compound of Type J:

### 5-iodo-2-pivalamidobenzoic acid

Triethylamine (4.9 mL, 35 mmol, 1 equiv.) and pivaloylchloride (6.0 mL, 49 mmol, 1.4 equiv.) were added to a solution of 2-amino-5-iodobenzoic acid (9.2 g, 35 mmol) in DCE (200 mL). The suspension was stirred for approx. 15 h at RT, then filtered, washed with water (3×) and dried. The product was isolated in virtually quantitative yield and used in the following synthesis step without further purification.

### Compound of Type K:

### 2-tert-butyl-6-iodo-4H-benzo[d][1,3]oxazine-4-one

5-iodo-2-pivalamidobenzoic acid (type J) was dissolved in acetic anhydride (70 mL, 2 mL/mmol) and distilled [initially mainly acetic acid was collected, later also acetic anhydride]. When distillation reached 140° C., distillation was conducted for a further 10 min. [If the temperature of 140° C. is not reached, then more acetic anhydride must be added.] The remaining anhydride was removed in vacuum. The residue crystallised out upon cooling to RT, was filtered out and used in the following synthesis step.

### Compound of Type L:

### 2-tert-butyl-6-iodoquinazoline-4(3H)-one

2-tert-butyl-6-iodo-4H-benzo[d][1,3]oxazine-4-one (type K) was added to 25% ammonia sol. (1.5 L) and stirred for approx. 15 h at RT. The filtered out product was suspended in 1% NaOH in ethylene glycol (250 mL) and held at 160° C. for 2 h. The mixture was then cooled to RT, the precipitate filtered out and washed with water. (Yield: 6.1 g, 60%).

### Compound of Type M:

### 2-tert-butyl-4-chloro-6-iodoquinazoline

2-tert-butyl-6-iodoquinazoline-4(3H)-one (type L) (656 mg, 2 mmol) was dissolved in POCl₃ (20 mmol, 10 equiv.) and DBU (1.33 mmol, 0.66 equiv.) added. The suspension was refluxed for approximately 3 h at 100° C. The reaction course was tracked by thin-film chromatography. The sol. was then placed on ice (100 mL) and the pH value set to neutral by adding sodium carbonate or sodium hydrogencarbonate (temperature 0-5° C.). The precipitated solid was filtered out, washed with plenty of water, dried and used in the next synthesis step.

### Compound of Type G:

### 2-tert-butyl-N-cyclopropyl-6-iodoquinazoline-4-amine

2-tert-butyl-4-chloro-6-iodoquinazoline (type M) was suspended in 1,4-dioxane (12 mL) and cyclopropylamine (1.2 mL, 10 mmol, 5 equiv.) added. The reaction mixture was stirred in a 70 mL SS autoclave for approx. 15 h at 80° C. (pressure ≦3-4 bar). As soon as complete conversion was reached, chloroform (50 mL) was added, the organic phase washed with water (2×100 mL) and evaporated to low bulk. (Yield: 621 mg, 85%).

2-tert-butyl-N-cyclopropyl-6-iodoquinazoline-4-amine (type G) was reacted with compound F to Example 187 (analogous process to the production of Example 186).

The syntheses of following examples were conducted in the same way as the synthesis of Example 187: Examples 180, 288, 289 and 293.

In the case of Example 181, the synthesis of the corresponding compound of type J was conducted as described below. All further synthesis steps were conducted in the same way as Example 187.

### Compound of Type G:

### 5-iodo-2-(2,2,2-trifluoroacetamido)benzoic acid

Triethylamine (4.9 mL, 35 mmol) was added to a sol. of 2-amino-5-iodobenzoic acid (9.2 g, 35 mmol) in DCE (200 mL), followed by trifluoroacetic anhydride (5.5 mL, 38.5 mmol, 1.1 equiv.). The suspension was stirred for approx. 15 h at RT, the product filtered out, washed with water (3×) and dried. The product was isolated in virtually quantitative yield and used in the following synthesis step.

### Synthesis Diagram for the Production of Compounds of Type H, by Way of Examples N4-cyclopropyl-6-iodo-N2,N2-dimethylquinazoline-2,4-diamine (H1) and N-cyclopropyl-6-iodo-2-morpholine quinazoline-4-amine (H2)

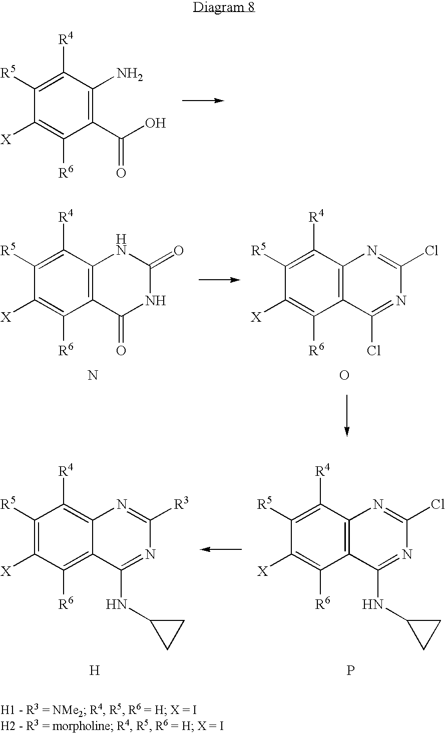

### Compound of Type N:

### 6-iodoquinazoline-2,4(1H,3H)-dione

2-amino-5-iodobenzoic acid (79 g, 300 mmol) was added to a mixture of water (2 L) and acetic acid (33 mL), the suspension was heated to 40° C. and then KOCN (48 g, 738 mmol) in water (200 mL) was carefully added. The reaction mixture was held at 40° C. for 8 h and then sodium hydroxide (540 g, 13.5 mmol, 45 equiv.) was added in portions so that a temperature of ≦40° C. was assured. The suspension was clear, the mixture was cooled to RT and the sodium salt filtered out. This was taken up in water (1 L), the pH value adjusted to 4 with HCl sol., the precipitate filtered out and the product dried in vacuum at 60° C. (Yield: 76.3 g, 88%).

### Compound of Type O:

### 2,4-dichloro-6-iodoquinazoline

6-iodoquinazoline-2,4(1H,3H)-dione (type N) (75 g, 265 mmol) was suspended in POCl₃ (500 mL) and dimethylaniline (22.6 mL, 177 mmol, 0.66 equiv.) added. The mixture was refluxed for 8 h and then placed on ice (1 L), wherein it was ensured that the temperature did not rise above 5° C. (addition of ice if necessary). The mixture was stirred for 30 min. at 0° C., heated to RT and the precipitate filtered out and washed with cold water until pH 7 was reached. The product was washed with hexane (2×) and recrystallised from isopropanol. (Yield: 50.6 g, 59%).

### Compound Type P:

### 2-chloro-N-cyclopropyl-6-iodoquinazoline-4-amine

2,4-dichloro-6-iodoquinazoline (type O) (50 g, 154 mmol) was suspended in acetonitrile (770 mL) and cyclopropylamine (23.5 mL, 338 mmol, 2.2 equiv.) added. The reaction mixture was stirred approx. 15 h at RT, filtered and washed with water (3×) and hexane. The product was dried, stored cold and converted further.

### Compound H1:

### N4-cyclopropyl-6-iodo-N2,N2-dimethylquinazoline-2,4-diamine

A sol. of 2-chloro-N-cyclopropyl-6-iodoquinazoline-4-amine (type P) (2.24 g, 6.5 mmol) in 1,4-dioxane (50 mL) was cooled to −(40-50)° C., the mixture saturated with dimethylamine and then sealed in a 20 ml SS autoclave and heated for approx. 15 h to 80° C. The reaction mixture was dissolved in chloroform, washed with water (2×) and the organic phase evaporated to low bulk. (Yield: 2.08 g, 91%).

[Direction also applies to volatile liquid amines; reaction temp. 60-150° C.; reaction time up to 15 h]

### Compound H2

### N-cyclopropyl-6-iodo-2-morpholine quinazoline-4-amine

Morpholine (1.9 g, 21.7 mmol, 2.5 equiv.) was added to 2-chloro-N-cyclopropyl-6-iodoquinazoline-4-amine (type P) (3 g, 8.7 mmol) in 1,4-dioxane (50 mL) and the mixture refluxed for 3-4 h. The mixture was then added to chloroform, washed with water (2×) and the organic phase evaporated to low bulk. (Yield: 2.6 g, 75%).

N4-cyclopropyl-6-iodo-N2,N2-dimethylquinazoline-2,4-diamine (H1) and N-cyclopropyl-6-iodo-2-morpholine quinazoline-4-amine (H2) were converted with compound F to Examples 186 (synthesis—Diagram 6) and 290 (analogous process to 186). The syntheses of following exemplary compounds were conducted in the same way as the syntheses of Examples 187 and 290: Examples 182-185 and 291.

### Synthesis Diagram for the Production of Compounds of Type I, with 6-bromo-N-cyclopropyl-7-fluoroquinazoline-4-amine as Example

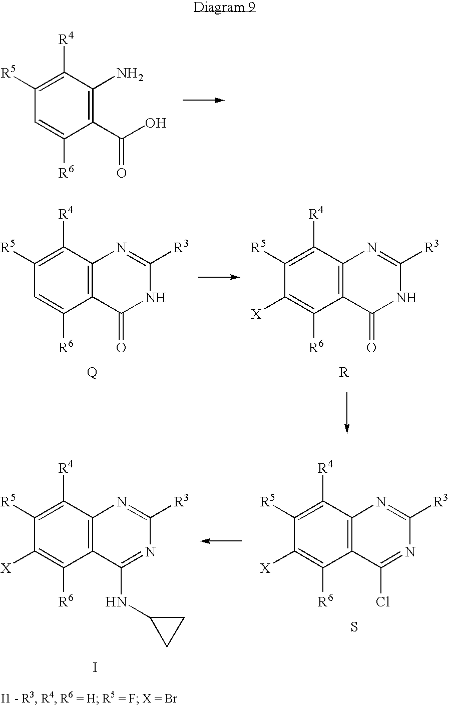

### Compound of Type Q:

### 7-fluoroquinazoline-4(3H)-one

2-amino-4-fluorobenzoic acid (13.5 g, 87 mmol) was dissolved in ethanol (15 mL), added to formamidine acetate (18.1 g, 174 mmol) and refluxed for 1 d. Water (250 mL) was then added, the precipitated product was filtered out and washed with 70% ethanol. (Yield: 14.2 g, 99%).

[Alternatively, the conversion was conducted in several cases (e.g. in the production of exemplary compound 881) without solvent, with formamide at 175° C.]

### Compound of Type R:

6-bromo-7-fluoroquinazoline-4(3H)-one

7-fluoroquinazoline-4(3H)-one (type Q) (14.2 g, 86 mmol) was suspended in water (250 mL), bromine (13.2 mL, 258 mmol) added and the mixture stirred 20 h at 80° C. The precipitate was filtered out and washed with water. (Yield: 4.5 g, 21%).

[Temperature and reaction times can vary slightly in the case of other substituents.]

### Compound of Type S:

### 6-bromo-4-chloro-7-fluoroquinazoline

6-bromo-7-fluoroquinazoline-4(3H)-one (type R) (4.5 g, 18 mmol) was suspended in POCl₃ (18 mL), DBU (1.9 mL, 12.7 mmol) added and the reaction mixture stirred 1 h at 100° C. The mixture was then concentrated by evaporation and the residue taken up in 150 mL of dichloromethane. It was extracted with water (150 mL) and 5% sodium hydroxide sol. (150 mL), the organic phase dried (MgSO₄) and evaporated to low bulk. (Yield: 4.2 g, 87%)

### Compound I1:

### 6-bromo-N-cyclopropyl-7-fluoroquinazoline-4-amine

6-bromo-4-chloro-7-fluoroquinazoline (type S) (4.2 g) was suspended in 1,4-dioxane (20 mL) and cyclopropylamine (3.3 mL, 48 mmol, 3 equiv.) then added. The reaction mixture was heated approx. 15 h at 80° C. in a 70 mL SS autoclave (pressure ≦3-4 bar). The mixture was then added to chloroform (100 mL) and the organic phase washed with water (2×100 mL) and evaporated to low bulk. The raw product was purified by column chromatography (chloroform:methanol, 50:1). (Yield: 1.4 g, 32%)

6-bromo-N-cyclopropyl-7-fluoroquinazoline-4-amine (I1) was converted with compound F to Example 882 (in the same way as Example 186).

The following Examples were produced in the same way as Example 882: Examples 881, 883, 884, 904 and 907.

Examples 905 and 906 were produced as follows: 6-bromo-N-cyclopropyl-7-fluoroquinazoline-4-amine (type I—Diagram 9) was produced as described above and then converted further in the same way as for the production of compound B (Diagram 3) (but 1,2-dimethoxyethane was used as solvent); the last 2 synthesis steps were conducted and in the same way as Example 211 (Diagram 3).

### Synthesis Diagram for the Production of Compounds of Type I Using 6-bromo-N-cyclopropyl-7-methoxyquinazoline-4-amine (I2) as Example

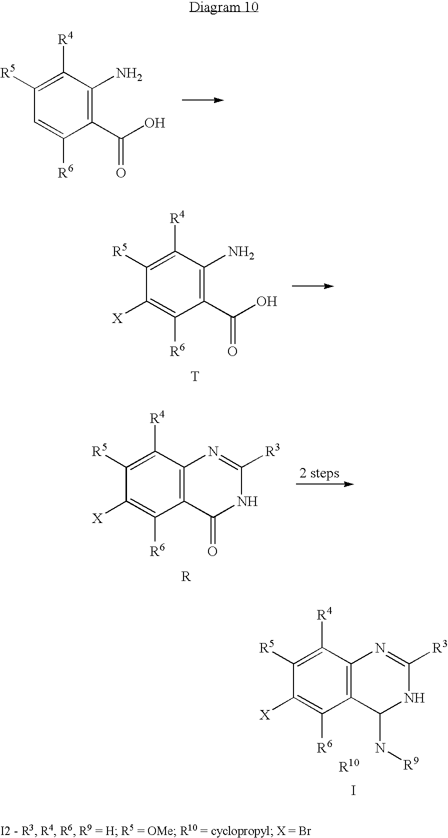

### Compound of Type T:

### 2-amino-5-bromo-4-methoxybenzoic acid

2-amino-4-methoxybenzoic acid (5 g, 30 mmol) was suspended in acetic acid (100 mL) and bromine (0.57 mL, 11.25 mmol) added at RT. The mixture was stirred approx. 15 h at RT, then additional bromine (0.2 mL, 3.75 mmol) was added and the reaction mixture held at 40° C. for 3 hours. The reaction course was tracked by thin-film chromatography, the product filtered out and washed with a little water. (Yield: 5.63 g, 77%)

### Compound of Type R:

### 6-bromo-7-methoxyquinazoline-4(3H)-one

2-amino-5-bromo-4-methoxybenzoic acid (type T) (5.63 g, 23 mmol) was dissolved in EtOH (100 mL), formamidine acetate (4.76 g, 46 mmol) added and refluxed for 1 d. Water (200 mL) was then added, the precipitated product filtered out and washed with 70% ethanol. (Yield: 4.14 g, 71%)

6-bromo-7-methoxyquinazoline-4(3H)-one (type T) was converted in the same way as the process already described into compound I2 (analogous to 6-bromo-N-cyclopropyl-7-fluoroquinazoline-4-amine (I1)—Diagram 9) and then converted with compound F to Example 899 (analogous to Example 186—Diagram 6).

Example 910 was produced in the same way as Example 899; however, the corresponding compound of type T (6-amino-3-bromo-2,4-difluorobenzoic acid) is commercially available.

### Synthesis Diagram for Examples 329 and 330

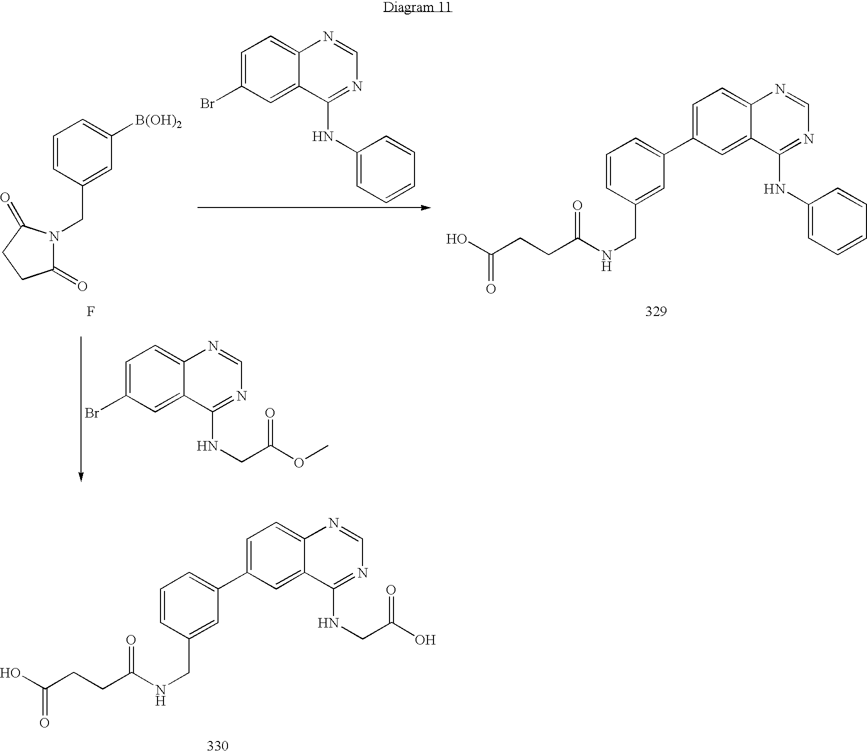

### Examples 329 and 330

### 4-oxo-4-(3-(4-(phenylamino)quinazolin-6-yl)benzylamino)butyric acid (329) 4-(3-(4-(carboxymethylamino)quinazolin-6-yl)benzylamino)-4-oxobutyric acid (330)

The synthesis was carried out according to the process described for compound B, in which the 6-bromo-quinazoline-4-amine compounds can be produced according to H. Hayashi et al., Bioorganic and Medicinal Chemistry, 2003, 11, 383 or in the same way as the compound of type I (Diagram 10) and compound F was produced as described in Diagram 6. After purification by column chromatography the products were obtained in good yields: (329): 0.500 g, 74%; (330): 0.790 g, >99%.

### Synthesis Diagram for Example 888

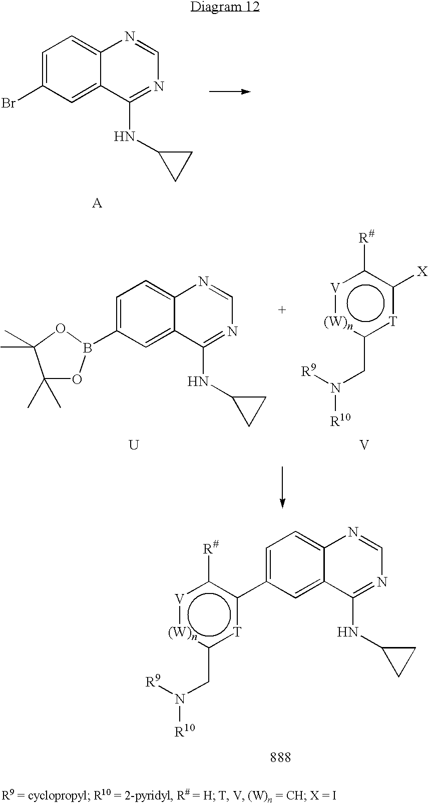

### Compound U:

### N-cyclopropyl-6-(4,4,5,5-tetramethyl-1,3,2-dioxaborolan-2-yl)quinazoline-4-amine

6-bromo-N-cyclopropylquinazoline-4-amine (A) (1.58 g, 6 mmol) (produced according to H. Hayashi et al., Bioorganic and Medicinal Chemistry, 2003, 11, 383 or in the same way as the compound of type I (Diagram 10), the corresponding compound of type T is commercially available), potassium acetate (1.8 g, 18.36 mmol), Pd(dppf)Cl₂ (100 mg) and bis-pinacolatodiboron (1.83 g, 7.2 mmol) were added to dimethylsulfoxide (36 mL) and heated 5 h to 80° C. The solution was extracted with chloroform (50 mL) and water (50 mL) and the organic phase washed with water again (50 mL), dried (MgSO₄) and evaporated to low bulk. The raw product was dissolved in 1,2-dimethoxyethane (5 mL) and this solution was used in Suzuki reactions with aryl halides (3 mmol).

### Example 888

### N-cyclopropyl-6-(3-((cyclopropyl(pyridin-2-yl)amino)methyl)phenyl)quinazoline-4-amine

N-cyclopropyl-N-(3-iodobenzyl)pyridine-2-amine (V1) (Diagram 13) (1.05 g, 3 mmol) was dissolved in 1,2-dimethoxyethane (25 mL) in argon and tetrakis(triphenyl-phosphine)palladium(0) (75 mg) added. The solution was stirred 10 min. at RT and then N-cyclopropyl-6-(4,4,5,5-tetramethyl-1,3,2-dioxaborolan-2-yl)quinazoline-4-amine (U) in 1,2-dimethoxyethane (5 mL), followed by 5% aqueous sodium carbonate sol. (10 mL) were added to the produced sol. The reaction mixture was stirred 2.5 h at 80° C. After cooling to RT it was extracted with chloroform (100 mL) and the organic phase washed with water, dried (MgSO₄) and evaporated to low bulk. The product was purified by column chromatography (1,2-dichloroethane:EtOH, 5:1). (Yield: 153 g, 13%)

### Synthesis Diagram for the Production of Compounds of Type V, on the Basis of Examples N-cyclopropyl-N-(3-iodobenzyl)pyridine-2-amine (V1), N-cyclopropyl-N-(3-iodobenzyl)aniline (V2), 1-(3-bromobenzyl)-3-methylimidazolidine-2-one (V3) and 1-(3-bromo-4-fluorobenzyl)pyrrolidine-2,5-dione (V4)

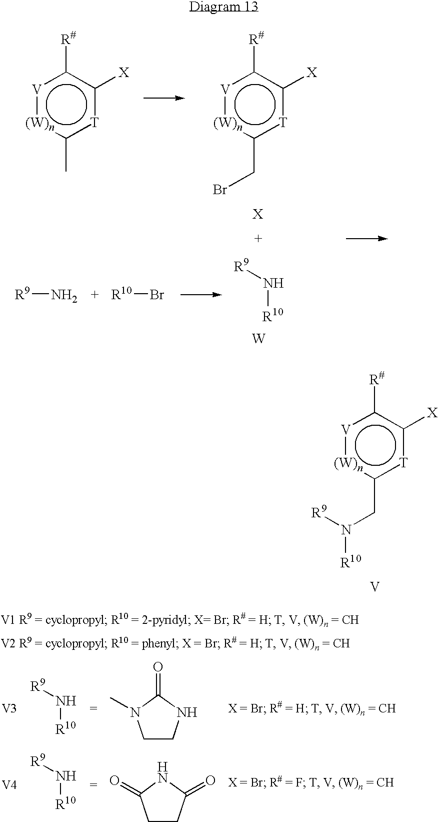

### Intermediate Steps Example 198

### Compound of Type W:

### N-cyclopropylpyridine-2-amine

2-bromopyridine (7.9 g, 50 mmol), sodium tert-butylate (7.1 g, 75 mmol), Pd₂(dba)₃ (1.83 g, 2 mmol), BINAP (372 mg, 0.6 mmol) and cyclopropylamine (5.5 mL, 80 mmol) were added together in toluene (60 mL) and heated in nitrogen in an 80 mL pressure vessel 24 h to 80° C. The mixture was cooled, diluted with diethyl ether, filtered over celite and evaporated to low bulk. The product was then purified by column chromatography (hexane:ethyl acetate, 1:2). (Yield: 1.2 g, 17%)

### Compound of Type V1:

### N-cyclopropyl-N-(3-iodobenzyl)pyridine-2-amine

Potassium tert-butylate (471 mg, 4.2 mmol) was added to a sol. of N-cyclopropylpyridine-2-amine (type W) (1.18 g, 4 mmol) in tetrahydrofuran (30 mL) and then a sol. of 1-(bromomethyl)-3-iodobenzole (type X, commercially available) (537 mg, 4 mmol) in tetrahydrofuran (20 mL) was added in drops over 1 h. The mixture was stirred 1 h, poured into water (100 mL) and extracted with chloroform (2×100 mL). The organic phase was dried (MgSO₄), evaporated to low bulk and the raw product purified by column chromatography (DCE:EtOH, 10:1). (Yield: 1.2 g, 86%)

N-cyclopropyl-6-(4,4,5,5-tetramethyl-1,3,2-dioxaborolan-2-yl)quinazoline-4-amine (U) was converted with N-cyclopropyl-N-(3-iodobenzyl)pyridine-2-amine (V1) to Example 888 as described in Diagram 11.

The following examples were produced in the same way as Example 888: Examples 876, 878, 885 (corresponding compounds of type W are commercially available for these examples).

### Example 908

For Example 908 the corresponding compound of type W was produced in the same way as the above-described process (N-cyclopropylpyridine-2-amine) and then converted to compound V2 as follows.

### Compound of Type V2:

### N-cyclopropyl-N-(3-iodobenzyl)aniline

N-cyclopropylaniline (type W, commercially available) (1.1 g) and 1-(bromomethyl)-3-iodobenzole (type X, commercially available) (2.5 g, 8.3 mmol) were dissolved in tetrahydrofuran (20 mL) and potassium carbonate (2.2 g, 16.6 mmol) added. The mixture was stirred approx. 15 h at RT and then placed on water (200 mL) and extracted with chloroform (100 mL). The organic phase was dried (MgSO₄) and evaporated to low bulk. (Yield: 600 mg, 21%)

N-cyclopropyl-N-(3-iodobenzyl)aniline (V2) was converted to Example 908 analogously to the process described for Example 888.

### Example 877

1-(3-bromobenzyl)-3-methylimidazolidine-2-one (V3) was converted to Example 877 analogously to the process described for Example 888.

### Compound of Type V3:

### 1-(3-bromobenzyl)-3-methylimidazolidine-2-one

1-methylimidazolidine-2-one (type W, commercially available) (2 g, 20 mmol) was dissolved in acetone (100 mL) and potassium carbonate (3 g, 20 mmol) added under constant stirring. 3-bromobenzylbromide (type X, commercially available) (2.5 g, 10 mmol) was added to the mixture and then refluxed for 24 h. Water (500 mL) was then added and extraction occurred with chloroform (3×200 mL). The organic phase was dried (MgSO₄), filtered and evaporated to low bulk. The raw product was purified by column chromatography (hexane:ethyl acetate, 3:2). (Yield: 812 mg, 30%)

Example 879 was produced from the corresponding compound of type W using the same process for 877.

### Example 895

### Compound of Type X

### 2-bromo-4-(bromomethyl)-1-fluorobenzole

2-bromo-1-fluoro-4-methylbenzole (6 mL, 48 mmol) and N-bromosuccinimide (2.7 g, 15 mmol) were dissolved in 1,2-dichloroethane (10 mL), 2,2′-azobisisobutyronitrile (16 mg, 0.1 mmol) was then added and the solution refluxed for 2 h. After the reaction mixture had cooled to RT, it was diluted with 1,2-dichloroethane (40 mL) and washed with water (2×50 mL). The organic phase was dried (MgSO₄) and evaporated to low bulk. (Yield: 9.2 g, 72%)

### Compound of Type V4:

### 1-(3-bromo-4-fluorobenzyl)pyrrolidine-2,5-dione

2-bromo-4-(bromomethyl)-1-fluorobenzole (type X) (1.96 g, 7.3 mmol) and succinimide (type W, commercial available) (1.5 g, 15 mmol) were dissolved in acetone (30 mL) and refluxed for 2 hours. After the reaction mixture had cooled to RT, it was added to water (100 mL). The acetone was removed and the aqueous phase extracted with chloroform (2×100 mL). The organic phase was dried (MgSO₄) and evaporated to low bulk and the product purified by column chromatography (hexane). (Yield: 1.55 g, 54%)

1-(3-bromo-4-fluorobenzyl)pyrrolidine-2,5-dione (V4) was converted to Example 895 analogously to the process described for Example 888.

The following examples were produced using analogous processes to Example 895: Examples 899 (corresponding compounds of types X and W, commercially available), 909 and 889 (in both cases the compounds of type X were produced using an analogous process; the compounds of type W are commercially available).

### Synthesis Diagram for the Production of Compounds of Type V on the Basis of Examples N-(3-iodobenzyl)-N-methylcyclopropanamine (V5) and N-cyclopropyl-N-(3-iodobenzyl)thiazole-2-amine (V6)

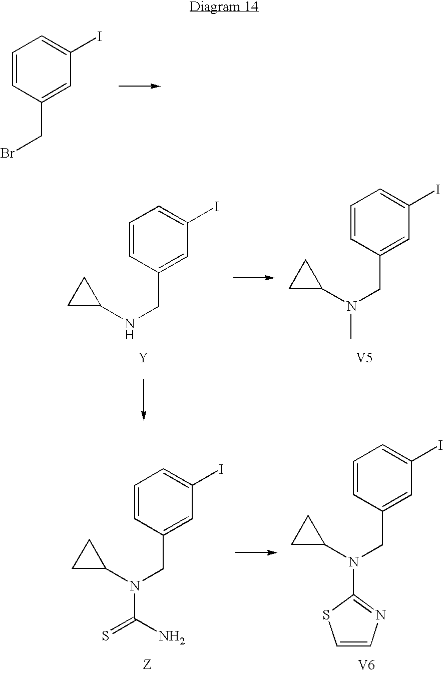

### Compound Y:

### N-(3-iodobenzyl)cyclopropanamine

Cyclopropylamine (27.7 mL, 400 mmol) was dissolved in tetrahydrofuran (100 mL) and 1-bromomethyl-3-iodobenzole (11.9 g, 40 mmol) in tetrahydrofuran (40 mL) was added in drops at 0° C. The reaction mixture was stirred for 20 h at RT, the product extracted with ethyl acetate (2×300 mL) and washed with water (200 mL). The organic phase was dried and evaporated to low bulk. (Yield: 9.4 g, 86%)

### Compound V5:

### N-(3-iodobenzyl)-N-methylcyclopropanamine

N-(3-iodobenzyl)cyclopropanamine (Y) (2.2 g, 8 mmol) and methyl iodide (0.5 mL, 8 mmol) were dissolved in dimethylformamide (10 mL, stored over molecular sieve (4 A)) and potassium carbonate (2.2 g, 16 mmol) added. The reaction mixture was stirred approx. 15 h, then sat. sodium hydrogencarbonate sol. (100 mL) was added and extraction occurred with chloroform (150 mL). The organic phase was dried (MgSO₄), evaporated to low bulk and purified by column chromatography (chloroform). (Yield: 1.6 g, 68%)

N-(3-iodobenzyl)-N-methylcyclopropanamine (V5) was converted to Example 891 analogously to the process described for Example 888.

### Compound Z:

### 1-cyclopropyl-1-(3-iodobenzyl)thiourea

N-(3-iodobenzyl)cyclopropanamine (Y) (5.3 g, 19.6 mmol) was added to a sol. of 1,1′-thiocarbonyldiimidazole (7 g, 32.1 mmol) and DIPEA (4.1 mL, 23.5 mmol) in acetonitrile (20 mL). The reaction mixture was stirred 4 h, conc. aqueous ammonia sol. (25 mL) was added and then stirred approx. 15 h. The product was extracted with chloroform (100 mL), the organic phase washed with water (100 mL), dried (MgSO₄) and purified by column chromatography (chloroform). (Yield: 4.0 g, 62%)

### Compound V6:

### N-cyclopropyl-N-(3-iodobenzyl)thiazole-2-amine

1-cyclopropyl-1-(3-iodobenzyl)thiourea (Z) (2.5 g, 7.4 mmol) and bromoacetaldehyde dimethylacetal (0.9 mL, 7.4 mmol) were dissolved in acetic acid (32 mL). The reaction mixture was refluxed for 1 h, added to water (100 ml) and slowly neutralised with 20% aqueous sodium hydroxide solution. The product was extracted with dichloromethane (2×100 mL) and washed with water (100 mL). The organic phase was dried (MgSO₄), evaporated to low bulk and purified by column chromatography (chloroform). (Yield: 1.9 g, 74%)

N-cyclopropyl-N-(3-iodobenzyl)thiazole-2-amine (V6) was converted to Example 892 analogously to the process described for Example 888.

### Synthesis Diagram for the Production of Compound of Type V, on the Basis of N-((4-bromothiazol-2-yl)methyl)-N-cyclopropylacetamide (V7)

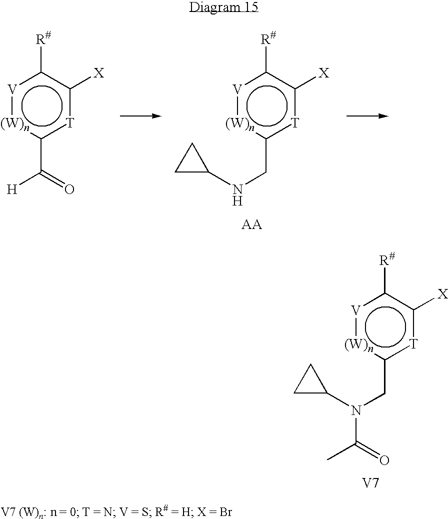

### Compound of Type AA:

### N-((4-bromothiazol-2-yl)methyl)cyclopropanamine

4-bromothiazole-2-carbaldehyde (2.5 g, 13 mmol) was dissolved in tetrahydrofuran (65 mL) and cyclopropylamine (3.72 mL, 53.7 mmol) and sodium triacetoxyborohydride (11.4 g, 53.7 mmol) added. The reaction mixture was stirred 20 h at RT and then hydrolysed at 0° C. with sat. sodium hydrogencarbonate sol. (150 mL). The product was extracted with ethyl acetate (2×300 mL) and the organic phase dried. (Yield: 3.0 g, 97%)

### Compound V7:

### N-((4-bromothiazol-2-yl)methyl)-N-cyclopropylacetamide

N-((4-bromothiazol-2-yl)methyl)cyclopropanamine (Type AA) (3.0 g) and triethylamine (2.67 mL, 19.1 mmol) were dissolved in 1,4-dioxane and a sol. of acetyl chloride (0.96 mL, 19.1 mmol) in 1,4-dioxane (20 mL) slowly added. The reaction mixture was stirred approx. 15 h, sat. sodium hydrogencarbonate sol. (100 mL) added and the product extracted with chloroform (150 mL). The organic phase was dried (MgSO₄), evaporated to low bulk and purified by column chromatography (1,2-dichloroethane:EtOH, 5:1). (Yield: 885 mg, 25%)

N-((4-bromothiazol-2-yl)methyl)-N-cyclopropylacetamide (V7) was converted to Example 903 analogously to the process described for Example 888.

N-((6-bromopyridin-2-yl)methyl)-N-cyclopropylacetamide (type V) was produced in the same way as N-((4-bromothiazol-2-yl)methyl)-N-cyclopropylacetamide (V6) and converted to Example 890 analogously to the process described for Example 888.

### Synthesis Diagram for the Production of Compound of Type V on the Basis of N-cyclopropyl-N-(3-iodo-2-methylbenzyl)acetamide (V9)

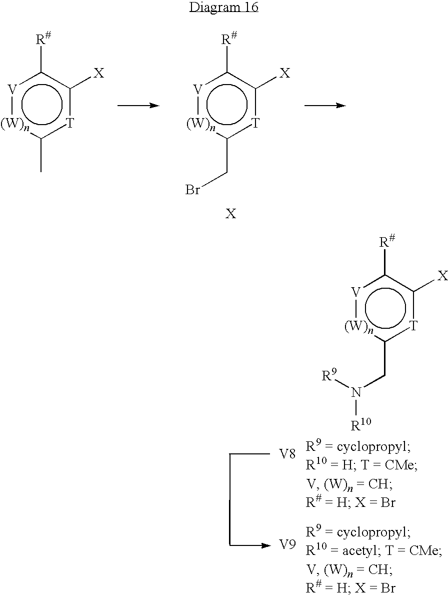

### Compound of Type X:

### 1-(bromomethyl)-3-iodo-2-methylbenzole

1-iodo-2,3-dimethylbenzole (0.7 mL, 5.0 mmol) and N-bromosuccinimide (890 mg, 5.0 mmol) were dissolved in 1,2-dichloroethane (10 mL), then 2,2′-azobisisobutyronitrile (16 mg, 0.1 mmol, 2 mol %) was added and the solution refluxed 2 h. After the reaction mixture had cooled to RT, it was diluted with 1,2-dichloroethane (40 mL) and washed with water (2×50 mL). The organic phase was dried (MgSO₄) and evaporated to low bulk and used in the following synthesis step. (Yield: 9.2 g, 72%)

### Compound V8:

### N-(3-iodo-2-methylbenzyl)cyclopropanamine

1-(bromomethyl)-3-iodo-2-methylbenzole (type X) was dissolved in tetrahydrofuran (15 mL) and cyclopropylamine (5 mL) added. The suspension was stirred approx. 15 h, filtered, diluted with 1,2-dichloroethane (50 mL) and washed with water (2×25 mL). The organic phase was dried (MgSO₄), filtered and evaporated to low bulk. (Yield: 1.31 g, 92%) [Triethylamine was used in some cases.]

### Compound V9:

### N-cyclopropyl-N-(3-iodo-2-methylbenzyl)acetamide

Triethylamine (0.95 ml, 6.8 mmol) was added to a sol. of N-(3-iodo-2-methylbenzyl)cyclopropanamine (V8) (1.31 mg, 4.6 mmol) in 1,2-dichloroethane (10 ml). The solution was cooled in ice water and acetyl chloride (0.34 ml, 4.8 mmol) added. The reaction mixture was stirred 8 h at RT, diluted with 1,2-dichloroethane (30 mL) and extracted with water (2×30 mL). The organic phase was dried (MgSO₄), filtered and evaporated to low bulk. (Yield: 900 mg, 60%) [In some cases 1,4-dioxane was used in place of 1,2-dichloroethane.]

N-cyclopropyl-N-(3-iodo-2-methylbenzyl)acetamide (V9) was converted to Example 911 analogously to the process described for Example 888.

The following examples were produced using analogous processes: Example 912 (purity 50%), 893 (corresponding compound of type X, commercially available), 896, 900 (amide formation to N-(3-bromo-4-fluorobenzyl)-2-cyano-N-cyclopropylacetamide (type V) with polymer-bonded carbodiimide—analogous to Example 211—Diagram 3), 897 and 902 (see 900).

### Synthesis Diagram for the Production of Example 875

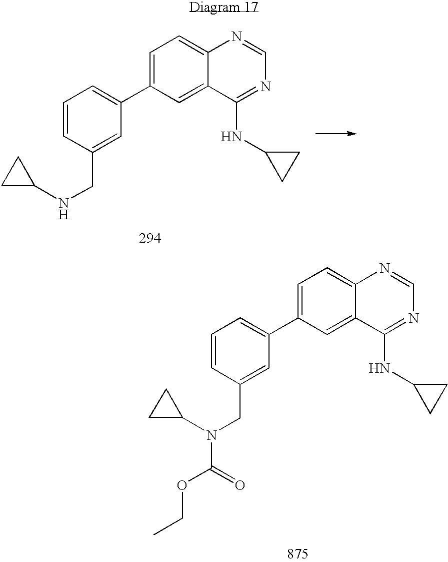

### Example 875

### Cyclopropyl-[3-(4-cyclopropylamino-quinazolin-6-yl)-benzyl]-carbamate

Cyclopropyl-[6-(3-cyclopropylaminomethyl-phenyl)-quinazolin-4-yl]-amine (294—Diagram 3) (330 mg, 3.5 mmol) was dissolved in 1,2-dichloroethane (10 mL) and DIPEA (260 μL, 1.5 mmol) added, followed by ethyl chloroformate (100 μL). The reaction mixture was stirred approx. 15 h at RT, 1,2-dichloroethane (40 mL) added and extraction occurred with sat. sodium carbonate sol. (50 mL). The organic phase was dried (MgSO₄), filtered, evaporated to low bulk and the product purified by column chromatography (chloroform/MeOH). (Yield: 157 mg, 39%)

The following examples were produced by analogous process to Example 875: Examples 886, 887, 894, 898.

### Synthesis of Examples 1-179 and 331-862 (Library Substances):

### Synthesis Directions for the Production of Non-Commercially Available Anthranilic Acids

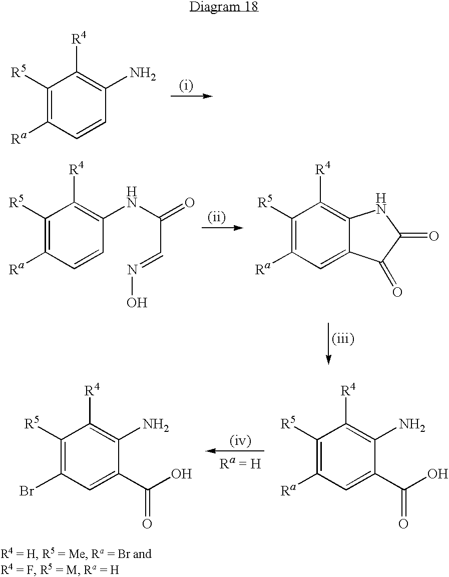

### Synthesis Step (i)

Na₂SO₄ (18 equiv.), the aniline (1 equiv., 2 mL/mmol), conc. HCl (0.25 mL/mmol) and a sol. of hydroxylamine hydrochloride in water (3 equiv., 3 mL/g) were added to a solution of chloral hydrate (1.1 equiv.) in water (1 mL/mmol). The reaction mixture was stirred 1 h at 80° C. and then cooled to RT. The desired product was extracted with EE, the organic phase washed with water and dried in a vacuum. The raw product was further used without further purification. (Yield approx. 70%)

### Synthesis Step (ii)

Concentrated sulfuric acid (10× educt) was heated to 50° C. and the raw product from step (i) added at 60-70° C. The reaction mixture was then heated to 80° C. and held at this temperature for 15 minutes. The mixture was then placed on ice and the aqueous phase extracted with EE and the organic phase was then washed with water and evaporated to low bulk. (Yield approx. 55%)

Synthesis Step (iii)

A 30% hydrogen peroxide sol. (2 mL/g) was added to a sol. of isatin derivative in 10 M of sodium hydroxide sol. (10 mL/g) at 70° C. and the reaction mixture was then held at this temperature for 1 h. The mixture was then cooled to 0° C., adjusted to pH 8 with conc. HCl, diluted with EE and then brought to pH 4-5. The organic phase was washed with water, dried (NaSO₄) and the solvent removed. (Yield approx. 60%)

### Synthesis Step (iv) (R^{a}=Hydrogen)

Bromine (1 equiv.) was slowly added to a sol. of anthranilic acid in CCl₄ (1 mL/mmol) and the mixture was stirred for 16 h at 10° C. When complete conversion was achieved (thin-film chromatography), the solvent was removed in vacuum and the solid thus obtained was washed with diethyl ether and dried. The product was used in step (v) without further purification (Yield approx. 85%)

### Synthesis Directions for the Production of the Amine Building Blocks of Anthranilic Acids and Commercially Available Boric Acids (Method A) (Syntheses of the Amine Building Blocks for Examples 1-179 and 331-862)

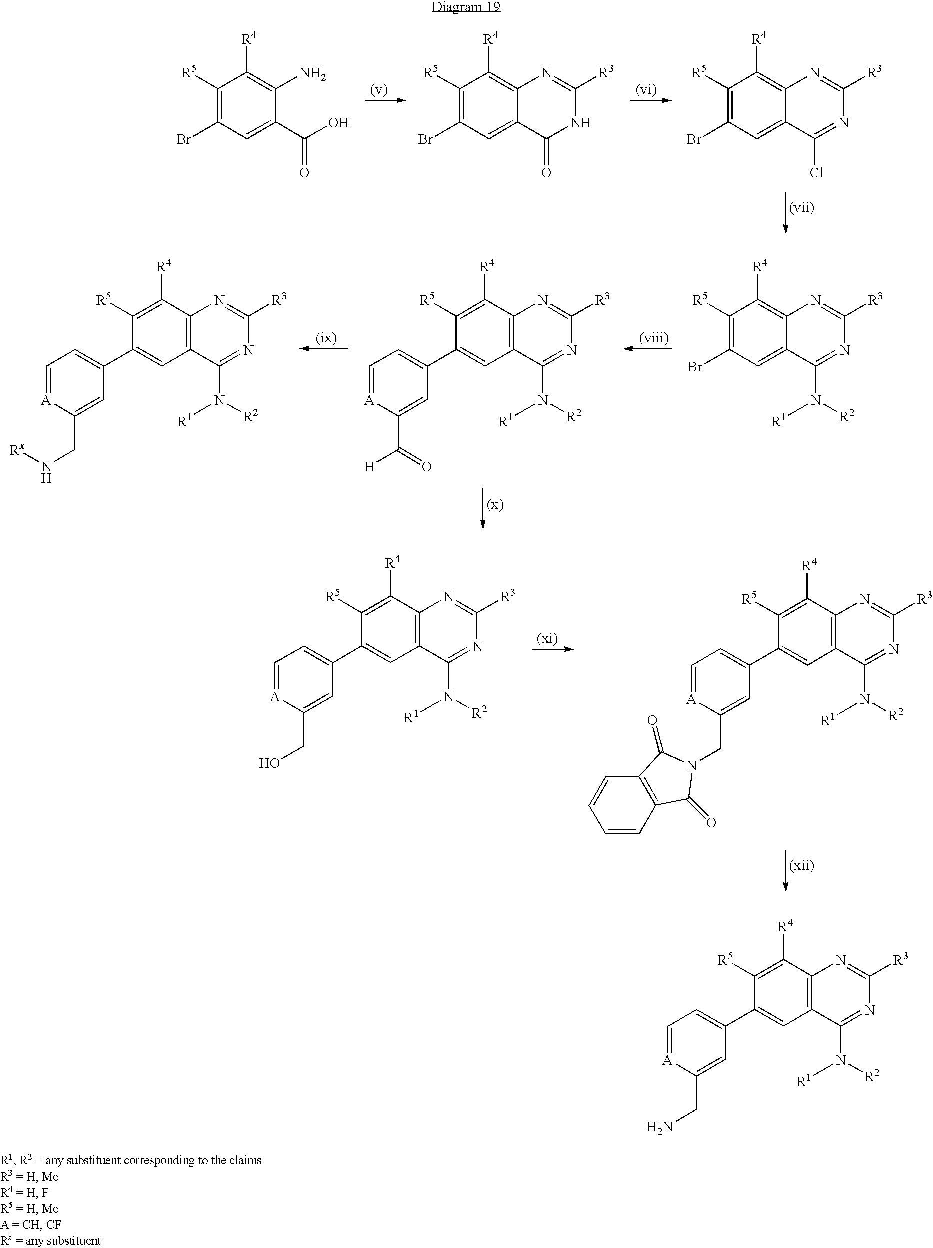

[Synthesis steps (i) to (iii) are optimized with respect to reaction time, equiv. and purification, not all amines were produced under these optimum conditions.]

### Synthesis Step (v)

### Method 1 (R³=Hydrogen)

The bromoanthranilic acid (commercially available or produced as described above) was refluxed for 1 hour in liquid ammonia (1 mL/g). The solution was then evaporated to low bulk and the residue mixed with formamide (2 mL/g) and refluxed 4 hours. Water was added, the mixture boiled for 30 min, and 20% sodium hydroxide sol. was then added until a clear solution formed. Ammonium carbonate was added to the hot solution until a precipitate formed. The mixture was held at 4° C. for 16 hours before the precipitate was filtered out, washed with water and dried in vacuum (yield 50-80%).

Method 2 (R³-methyl)

The bromoanthranilic acid was refluxed in liquid ammonia (10 mL/g) for 1 h. The solution was then evaporated to low bulk and the residue mixed with acetic anhydride (2 mL/g) and refluxed 4 h. Water was added, the mixture boiled for 30 min and 20% sodium hydroxide sol. was then added until a clear solution formed. Ammonium carbonate was added to the hot solution until a precipitate formed. The mixture was held at 4° C. for 16 h before the precipitate was filtered out, washed with water and dried in vacuum (yield 60%)

### Synthesis Step (vi)

### Method 1 (R³=Hydrogen)

Thionyl chloride (25 mL/g) was added in drops to the cyclic substance at 0° C. with constant stirring, followed by a kat quantity of DMF. The reaction mixture was then refluxed 16 hours, the reaction course tracked by thin-film chromatography and the mixture evaporated to low bulk after the reaction has finished. The raw product was directly converted further. (Yield approx. 80%)

### Method 2 (R³=Hydrogen and R⁵=Methyl or R³=Methyl and R⁵=Hydrogen)

N,N-dimethylaniline (1.5 equiv.) and POCl₃ (1 equiv.) were added to a sol. of the cyclic substance in benzole (20 mL/g) and the resulting mixture refluxed 6 h. After complete conversion of the educt (thin-film chromatography), the organic phase was extracted with water, 1% sodium hydroxide sol., water, brine, 10% HCl sol., water and brine, dried (Na₂SO₄) and evaporated to low bulk. The raw product was directly converted further. (Yield approx. 70%)

Synthesis Step (vii) (Intermediate for Examples 1-179 and 331-862)

The chloroquinazoline compound was taken up in isopropanol? (10 mL/g raw product) and triethylamine or DIPEA (1.2-1.5 equiv.) and the corresponding amine (R^{x}NH₂) (1.1-1.2 equiv.) added. The reaction mixture was then stirred 5-16 h at RT. The solvent was removed and the solid residue washed with water and dried. The raw product was crystallised from 20% EE in hexane. (Yield 60-80%)

Synthesis Step (viii)
Method 1 (Intermediate for Examples Selected from 1-179 and 331-332, in which A=CH, CF)

The bromoquinazoline, boric acid (1.2 equiv.) and sodium carbonate (2.5 equiv.) were taken up in a mixture of EtOH (2 mL/mmol), toluene (4 mL/mmol) and water (2 mL/mmol). Argon was directed through the reaction mixture, then tetrakis(triphenyl-phosphine)palladium(0) (20% by wt) added and the mixture heated 16 h in argon to 80° C. The reaction mixture was cooled to RT, filtered over celite and washed with toluene. The filtrate was evaporated to low bulk and the residue purified by column chromatography (30% acetone in hexane) (yield 40-50%).

Method 2 or 3 (Intermediate for Examples Selected from 363-862, in which A=CH, CF)

### Method 2

Tetrakis(triphenylphosphine)-palladium(0) (0.05 equiv.) was added in argon to a DMF sol. of the bromoquinazoline (1 equiv.), boric acid (1.2 equiv.) and sodium carbonate (2.5 equiv.), and the mixture heated to 80° C. for 16 h. When the complete conversion of the bromine compound (LCMS) had been achieved, the mixture was filtered over celite and washed with EE. The filtrate was washed with water and brine, dried (Na₂SO₄) and evaporated to low bulk. The raw product was purified by column chromatography (20-30% EE in DCM). (Yield 40-60%)

### Method 3

### Production of 5-(4-(tert-butylamino)quinazolin-6-yl)-2-fluorobenzaldehyde and 3-(4-(cyclopropylamino)-7-methylquinazolin-6-yl)benzaldehyde

Tris(dibenzylideneacetone)palladium(0) (0.025 equiv.) was added in argon to a 1,4-dioxane sol. of the quinazoline compound (1 equiv.), boric acid (1.2 equiv.), cesium carbonate (2.5 equiv.) and Xanthpos (0.075 equiv.), and the mixture heated 6-10 h to 80° C. When the complete conversion of the bromine compound (LCMS) had been achieved, the mixture was filtered over celite and washed with EE. The filtrate was washed with water and brine, dried (Na₂SO₄) and evaporated to low bulk. The raw product was purified by column chromatography (approx. 30% EE in DCM). (Yield approx. 50%)

### Synthesis Step (ix)

Method 1 (Intermediate for Examples Selected from 1-179 and 331-332, in which A=CH, CF)

The corresponding amine (R^{x}NH₂) (1 equiv.) was added to a stirred sol. of the quinazoline compound in 1% acetic acid in MeOH (8 mL/mmol) at 0° C. in a nitrogen atmosphere, followed by sodium cyanoborohydride (1.2 equiv.). The reaction mixture was heated to RT and then stirred 16 h. The solvents were removed in vacuum, the residue taken up in EE and extracted with sat. sodium hydrogencarbonate sol. and brine, dried (Na₂SO₄) and evaporated to low bulk. The raw product was purified by column chromatography (80% acetone in DCM) (yield 70-80%).

Methods 2 and 3 (Intermediate for Examples Selected from 363-862, in which A=CH, CF)

### Method 2

The corresponding amine (R^{x}NH₂) (1.1 equiv.), molecular sieve (4 A) (4× aldehyde) were added to a sol. of the quinazoline compound in DCM (8 mL/g) in nitrogen and the reaction mixture stirred for 6 h at 25° C. The reaction mixture was filtered and evaporated to low bulk in a vacuum. The residue was taken up in MeOH (10× aldehyde) and cooled to 0° C. Sodium borohydride (0.75 equiv.) was added in portions to this sol. and the mixture then stirred for 16 h at 25° C. Ice water was added, the MeOH removed in vacuum and the residue taken up in EE and washed with water and brine, dried (Na₂SO₄) and in turn evaporated to low bulk. The raw product was purified by column chromatography (20-30% acetone in DCM) (yield 40-50%).

### Method 3

6-(3-((2-fluorophenylamino)methyl)phenyl)-N-methylquinazoline-4-amine 2-fluoroaniline (1 equiv.), acetic acid (2 equiv.) and sodium triacetoxyborohydride (2.5 equiv.) were added to a solution of 3-(4-(methylamino)quinazolin-6-yl)benzaldehyde (1.2 equiv.) in DCM (5 mL/mmol) in nitrogen and the mixture then stirred 16 h at 25° C. After the starting material had been completely converted (LCMS), the organic phase was washed with sodium carbonate sol. and brine and then dried (Na₂SO₄). The organic phase was evaporated to low bulk and the raw product purified by column chromatography (20% acetone in DCM).

### Synthesis Step (x) (Intermediate for Examples 333-362, A=CH)

Sodium borohydride (2 equiv.) was added slowly in portions to a stirred sol. of the quinazoline in MeOH (5 mL/mmol) at 0° C. The reaction mixture was stirred for 6 h at RT, the solvent was then removed, the residue diluted with ice water and extracted with EE. The organic phase was washed with brine, dried (Na₂SO₄) and evaporated to low bulk. The raw product was purified by column chromatography (3% MeOH in DCM) (yield 80-90%).

### Synthesis Step (xi) (Intermediate for Examples 333-362, A=CH)

Phthalimide (1 equiv.) was added at 0° C. to a stirred sol. of the quinazoline compound in THF (10 mL/mmol), followed by triphenylphosphine (1.2 equiv.). DEAD (1, 2 equiv.) was then slowly added in drops to the sol. and the reaction mixture stirred for 18 h at RT. Sat. ammonium chloride sol. was then added and the mixture extracted with EE. The organic phase was washed with brine, dried (Na₂SO₄) and evaporated to low bulk. The raw product was purified by column chromatography (30% acetone in DCM) (yield 65-70%).

Synthesis Step (xii) (Intermediate for Examples 333-362, A=CH)

The quinazoline compound was dissolved in EtOH (20 mL/mmol) and hydrazine hydrate (3 equiv.) added. The reaction mixture was then refluxed 5 h. The mixture was then cooled to RT, filtered and washed with EtOH (yield 60-70%).

### Synthesis Directions for the Production of the Amine Building Blocks of Anthranilic Acids (Method B—Boric Acids not Commercially Available)

(Examples Selected from 363-862, in which A=N, CCl)

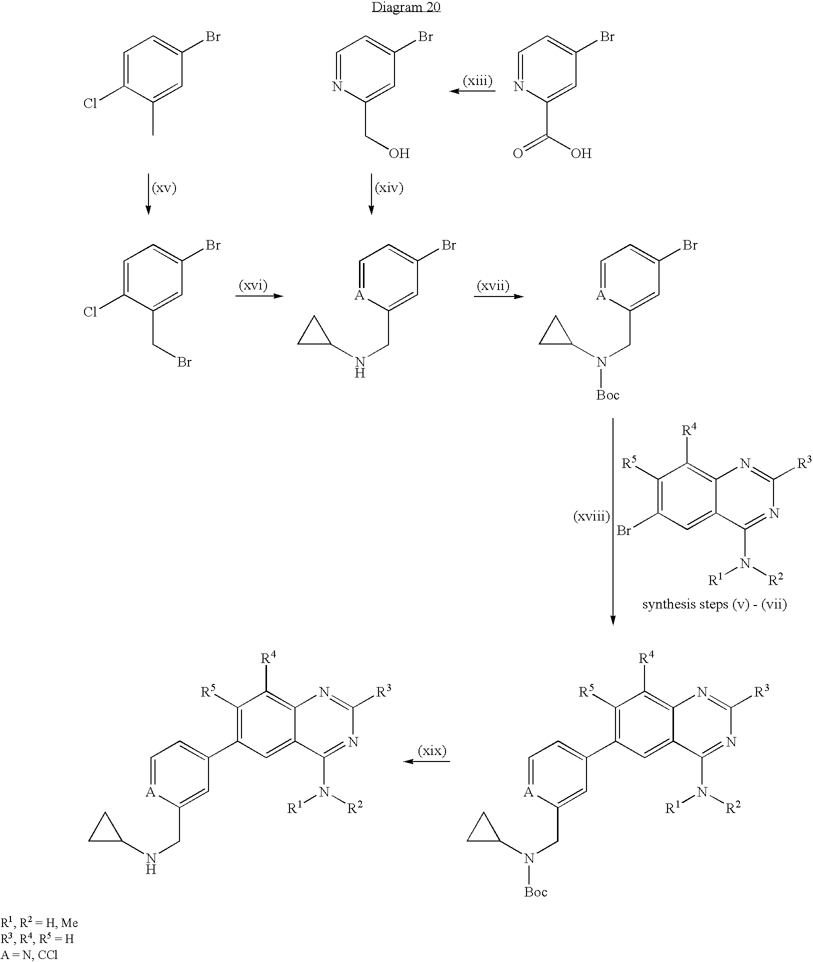

Synthesis Step (xiii)

Triethylamine (1.05 equiv.) was added to a suspension of 4-bromopicolinic acid in benzole (5 mL/mmol), followed by chloroformate (1.05 equiv.) and the reaction mixture stirred 1 h at RT. Triethylamine hydrochloride was filtered out and the filtrate evaporated to low bulk. The anhydride thus obtained was taken up in THF (5 mL/mmol) and added in drops to a suspension of lithium aluminium hydride (1 equiv.) in THF (2 mL/mmol) at −78° C. The mixture was stirred for 30 min. at −78° C., then sat. Na₂SO₄ sol. was added, filtered over celite, washed with EE and the organic phase was evaporated to low bulk. The raw product thus obtained was purified by column chromatography (30% EE in hexane) (yield approx. 50%).

Synthesis Step (xiv)

Triethylamine (2.5 equiv.) and methane sulfonylchloride (1.2 equiv.) were added to a sol. of 4-bromo-2-pyridyl methylalcohol in DCM (3 mL/mmol) at 0° C. and the mixture stirred 1 h. The reaction course was tracked by column chromatography and as soon as complete conversion was reached the mixture was diluted with DCM, washed with water and brine, dried (Na₂SO₄) and evaporated to low bulk. The raw product was then dissolved in THF (3 mL/mmol), cyclopropylamine (5 equiv.) was added and the mixture refluxed for 6 h. As soon as complete conversion was reached the solvent was removed and the residue taken up in EE, washed with water and brine and dried (Na₂SO₄). The organic phase was evaporated to low bulk in a vacuum and the raw product purified by column chromatography (30% EE in DCM) (yield approx. 65%).

### Synthesis Step (xv)

N-bromosuccinimide (1.1 equiv.) and benzoylperoxide (0.002 equiv.) were added to a sol. of 2-chloro-5-bromotoluene in CCl₄ (5 mL/mmol) and the mixture refluxed for 2 h. After complete conversion (thin-film chromatography), the reaction mixture was cooled, filtered and washed with chloroform. The organic phase was evaporated to low bulk in a vacuum and the raw product purified by column chromatography (5% EE in hexane) (yield approx. 65%).

Synthesis Step (xvi)

The bromine derivative was taken up in EtOH (10 mL/g) and mixed with cyclopropylamine (5 equiv.). The mixture was refluxed 3 h, EtOH removed and the residue taken up in EE. The organic phase was washed in water and brine, dried (Na₂SO₄) and evaporated to low bulk. The raw product was purified by column chromatography (10% EE in DCM) (yield approx. 80%).

Synthesis Step (xvii)

The amine was dissolved in dioxane (3 mL/mmol) and Boc anhydride (1.5 equiv.) and 2% sodium carbonate sol. (1:1/dioxane: sodium carbonate sol.) were added. The reaction mixture was stirred for 2 h at 25° C. After complete conversion had been achieved, the mixture was diluted with EE and washed with water and brine and dried (Na₂SO₄). The organic phase was evaporated to low bulk in a vacuum and the raw product purified by column chromatography (30% EE in hexane) (yield approx. 80%).

Synthesis Step (xviii)

Bis(dipinacolato)diborane (1 equiv.), potassium acetate (2.5 equiv.) and palladium(II)chloride (0.05 equiv.) and dppf (0.05 equiv.) were added to a sol. of the bromine derivative (1 equiv.) in DMF (6 mL/mmol) in argon, and the reaction mixture was then stirred for 2 hours at 60° C. The mixture was then cooled to RT and the bromoquinazoline compound (product from synthesis step (vii)) (1 equiv.), palladium(II)chloride (0.05 equiv.), dppf (0.05 equiv.) and 2 M sodium carbonate sol. (3 mL/mmol) were added. The reaction mixture was stirred for 3 h at 80° C. and then filtered over celite. The filtrate was diluted with EE, washed with water and brine and dried (Na₂SO₄). The organic phase was evaporated to low bulk in a vacuum and the raw product purified by column chromatography (10% acetone in DCM) (yield approx. 45%).

Synthesis Step (xix)

The protecting groups were eliminated using known methods:
1. Philip J. Kocienski, Protecting Groups, 3rd Edition, Georg Thieme Verlag, 2005
2. Peter G. M. Wuts, Theodora W. Greene, Protecting groups in Organic Synthesis, 4th Edition, Wiley Interscience, 2007.
   General Directions for the Production of Compounds from the Amine Building Blocks: (Examples 1-179 and 331-862)

### 1. Conversion of Carboxylic Acids (xx)

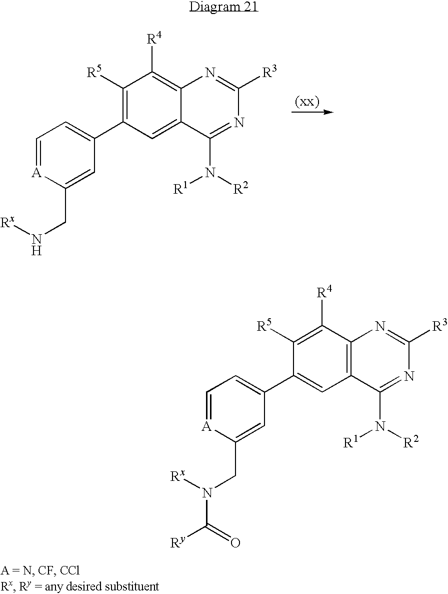

The selected carboxylic acids (1.1 equiv.), EDCI (1.2 equiv.), HOBt (1 equiv.) and DIPEA (1.5 equiv.) were each mixed in 0.5 mL of DCM in reaction vessels and shaken for 30 min.×50 mg [X=number of reactions] of the amine were dissolved in X 0.5 mL of DCM and evenly distributed over the reaction vessels with the carboxylic acids. The reaction mixtures were then shaken for 16 h at RT. Processing occurred by adding DCM, followed by extraction with ammonium chloride sol., sodium hydrogencarbonate sol. and brine. The organic phases were then dried (Na₂SO₄) and evaporated to low bulk. The raw products were purified by means of a Biotage parallel purification system. (Mobile phase in most cases: DCM/MeOH).

2. Conversion with Sulfonyl Chlorides (xxi)

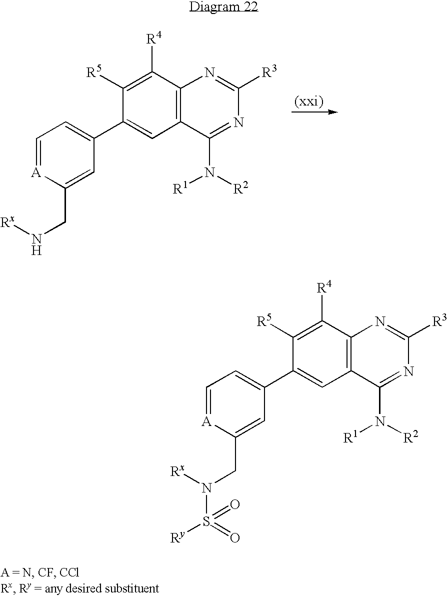

The sulfonyl chlorides (1.2 equiv.) were converted with the amines (1 equiv., 0.025 mmol) in the presence of DIPEA (2.5 equiv.) in DCM (3 mL/mmol) to the corresponding sulfonamides. The raw products were purified using a Biotage parallel purification system.

The library substances (Examples 1-179 and 331-862) were analyzed by mass spectroscopy (Table 1).

Examples 866, 867, 871-873 were produced analogously to Example 192. Examples 868 and 870 were produced analogously to Example 211. Example 869 was produced from Example 297 and 1-(tert-butoxycarbonylamino)cyclopropanecarboxylic acid (commercially available) by an amide formation (EDCI—see also Examples 1-179 and 331-862 step (xx)), followed by elimination of Boc protecting groups (analogously to Example 297); which analogously to Example 252 was in turn converted with pentanoyl chloride to Example 874.

### Examples 329 and 330

### 4-oxo-4-(3-(4-(phenylamino)quinazolin-6-yl)benzylamino)butyric acid (329) 4-(3-(4-(carboxymethylamino)quinazolin-6-yl)benzylamino)-4-oxobutyric acid (330)

The synthesis was carried out by the process described for compound B. After purification by column chromatography, the products were obtained in good yields: (329): 0.500 g, 74%; (330): 0.790 g, >99%.

The synthesis of Examples 196, 198, 199, 285-287 and 296 was carried out in the same way as the synthesis of compound B from the corresponding boric acid.

The synthesis of Examples 193, 194, 236, 242 and 243 was conducted in the same way as the synthesis of Example 192.

The synthesis of Examples 299-310, 304-310, 312-315 and 318-327 was conducted in the same way as the synthesis of Example 294.

The synthesis of Examples 180, 190, 191, 195, 197, 200-205, 207-209, 213, 214, 216, 218, 219, 221, 230, 232, 233, 235, 237-240, 244, 248, 249, 253-257, 259-269, 271-275, 277, 279-284, 302, 303, 311, 316 and 317 was conducted in most cases in the same way as the syntheses of Example 252 or 211. However, in just a few cases alternative solvents and coupling reagents, in particular pentafluorophenyl trifluoroacetate, were used.

The synthesis of Examples 210, 215, 222, 223, 226, 227, 231, 234, 246, 250, 251, 270 and 276 was achieved using the processes described for Example 225.

The synthesis of Examples 189, 193 and 278 was achieved using the processes described for Example 192.

The synthesis of Examples 194, 220, 224, 228, 229, 243, 245 and 258 was achieved using the processes described for Example 241.

The synthesis of Example 217 was achieved using the processes described for Example 206.

In the case of analogous production processes it is evident to persons skilled in the art which starting compounds and intermediate products must be used in each case to arrive at the corresponding Example. The corresponding mass-spectrometric data or NMR data of the synthesised compounds are set forth below:

TABLE 1

| | | MS |
|---|---|---|
| | | (m/z) |
| Example | | [M + H]⁺ |
| 1 | 6-hydroxy-N-isobutyl-N-[3-(4-morpholin-4-yl-quinazolin-6-yl)- | 498.3 |
| | benzyl]-nicotinamide | |
| 2 | N-cyclopropyl-6-hydroxy-N-[3-(4-pyrrolidin-1-yl-quinazolin-6-yl)- | 466.2 |
| | benzyl]-nicotinamide | |
| 3 | N-cyclopropyl-N-[3-(4-cyclopropylamino-quinazolin-6-yl)- | 471.2 |
| | benzyl]-2,4-difluorobenzamide | |
| 4 | thiophene-3-carboxylic acid cyclopropyl-[3-(4-cyclopropylamino- | 441.2 |
| | quinazolin-6-yl)-benzyl]-amide | |
| 5 | N-isobutyl-N-[3-(4-morpholin-4-yl-quinazolin-6-yl)-benzyl]- | 481.3 |
| | benzamide | |
| 6 | 2-bromo-N-isobutyl-N-[3-(4-morpholin-4-yl-quinazolin-6-yl)- | 559.2 |
| | benzyl]-benzamide | |
| 7 | N-isobutyl-3,4-dimethoxy-N-[3-(4-morpholin-4-yl-quinazolin-6- | 541.3 |
| | yl)-benzyl]-benzamide | |
| 8 | 2,4-difluoro-N-isobutyl-N-[3-(4-morpholin-4-yl-quinazolin-6-yl)- | 517.2 |
| | benzyl]-benzamide | |
| 9 | isoquinoline-3-carboxylic acid isobutyl-[3-(4-morpholin-4-yl- | 532.3 |
| | quinazolin-6-yl)-benzyl]-amide | |
| 10 | N-isobutyl-N-[3-(4-morpholin-4-yl-quinazolin-6-yl)-benzyl]-3- | 507.3 |
| | phenyl-acrylamide | |
| 11 | 4-chloro-N-isobutyl-N-[3-(4-morpholin-4-yl-quinazolin-6-yl)- | 515.2 |
| | benzyl]-benzamide | |
| 12 | 4-cyano-N-isobutyl-N-[3-(4-morpholin-4-yl-quinazolin-6-yl)- | 506.3 |
| | benzyl]-benzamide | |
| 13 | pyrazine-2-carboxylic acid isobutyl-[3-(4-morpholin-4-yl- | 483.3 |
| | quinazolin-6-yl)-benzyl]-amide | |
| 14 | 6-bromo-pyridine-2-carboxylic acid isobutyl-[3-(4-morpholin-4-yl- | 560.2 |
| | quinazolin-6-yl)-benzyl]-amide | |
| 15 | N-isobutyl-N-[3-(4-morpholin-4-yl-quinazolin-6-yl)-benzyl]-2- | 511.3 |
| | phenoxy-acetamide | |
| 16 | N-isobutyl-N-[3-(4-morpholin-4-yl-quinazolin-6-yl)-benzyl]-2- | 496.3 |
| | pyridin-2-yl-acetamide | |
| 17 | 3-cyano-N-isobutyl-N-[3-(4-morpholin-4-yl-quinazolin-6-yl)- | 506.3 |
| | benzyl]-benzamide | |
| 18 | thiophene-3-carboxylic acid isobutyl-[3-(4-morpholin-4-yl- | 487.2 |
| | quinazolin-6-yl)-benzyl]-amide | |
| 19 | 6-chloro-N-isobutyl-N-[3-(4-morpholin-4-yl-quinazolin-6-yl)- | 516.2 |
| | benzyl]-nicotinamide | |
| 20 | 3-methyl-thiophene-2-carboxylic acid isobutyl-[3-(4-morpholin-4- | 501.2 |
| | yl-quinazolin-6-yl)-benzyl]-amide | |
| 21 | 2-cyano-N-isobutyl-N-[3-(4-morpholin-4-yl-quinazolin-6-yl)- | 444.2 |
| | benzyl]-acetamide | |
| 22 | N-isobutyl-4-methyl-N-[3-(4-morpholin-4-yl-quinazolin-6-yl)- | 495.3 |
| | benzyl]-benzamide | |
| 23 | N-isobutyl-N-[3-(4-morpholin-4-yl-quinazolin-6-yl)-benzyl]-2- | 549.2 |
| | trifluoromethyl-benzamide | |
| 24 | 5-bromo-thiophene-2-carboxylic acid-[3-(4-morpholin-4-yl- | 565.1 |
| | quinazolin-6-yl)-benzyl]-amide | |
| 25 | N-isobutyl-N-[3-(4-morpholin-4-yl-quinazolin-6-yl)-benzyl]- | 482.3 |
| | isonicotinamide | |
| 26 | N-isobutyl-N-[3-(4-morpholin-4-yl-quinazolin-6-yl)-benzyl]- | 447.3 |
| | butyramide | |
| 27 | 2-hydroxy-N-isobutyl-N-[3-(4-morpholin-4-yl-quinazolin-6-yl)- | 497.3 |
| | benzyl]-benzamide | |
| 28 | 2-fluoro-N-isobutyl-N-[3-(4-morpholin-4-yl-quinazolin-6-yl)- | 567.2 |
| | benzyl]-4-trifluoromethyl-benzamide | |
| 29 | thiophene-2-carboxylic acid isobutyl-[3-(4-morpholin-4-yl- | 487.2 |
| | quinazolin-6-yl)-benzyl]-amide | |
| 30 | N-isobutyl-N-[3-(4-morpholin-4-yl-quinazolin-6-yl)-benzyl]- | 482.3 |
| | nicotinamide | |
| 31 | 2-dimethylamino-N-isobutyl-N-[3-(4-morpholin-4-yl-quinazolin-6- | 462.3 |
| | yl)-benzyl]-acetamide | |
| 32 | N-isobutyl-4-methoxy-N-[3-(4-morpholin-4-yl-quinazolin-6-yl)- | 511.3 |
| | benzyl]-benzamide | |
| 33 | 2-chloro-N-isobutyl-N-[3-(4-morpholin-4-yl-quinazolin-6-yl)- | 515.2 |
| | benzyl]-benzamide | |
| 34 | furan-2-carboxylic acid isobutyl-[3-(4-morpholin-4-yl-quinazolin- | 471.2 |
| | 6-yl)-benzyl]-amide | |
| 35 | pyridine-2-carboxylic acid isobutyl-[3-(4-morpholin-4-yl- | 482.3 |
| | quinazolin-6-yl)-benzyl]-amide | |
| 36 | N-benzyl-N-[3-(4-isopropylamino-quinazolin-6-yl)-benzyl]- | 487.2 |
| | benzamide | |
| 37 | N-benzyl-2-bromo-N-[3-(4-isopropylamino-quinazolin-6-yl)- | 565.2 |
| | benzyl]-benzamide | |
| 38 | 3-methyl-but-2-ene acid benzyl-[3-(4-isopropylamino-quinazolin- | 465.3 |
| | 6-yl)-benzyl]-amide | |
| 39 | N-benzyl-N-[3-(4-isopropylamino-quinazolin-6-yl)-benzyl]-3,4- | 547.3 |
| | dimethoxy-benzamide | |
| 40 | N-benzyl-2,4-difluoro-N-[3-(4-isopropylamino-quinazolin-6-yl)- | 523.2 |
| | benzyl]-benzamide | |
| 41 | isoquinoline-3-carboxylic acid benzyl-[3-(4-isopropylamino- | 538.3 |
| | quinazolin-6-yl)-benzyl]-amide | |
| 42 | N-benzyl-N-[3-(4-isopropylamino-quinazolin-6-yl)-benzyl]-3- | 513.3 |
| | phenyl-acrylamide | |
| 43 | N-benzyl-4-chloro-N-[3-(4-isopropylamino-quinazolin-6-yl)- | 521.2 |
| | benzyl]-benzamide | |
| 44 | N-benzyl-4-cyano-N-[3-(4-isopropylamino-quinazolin-6-yl)- | 512.2 |
| | benzyl]-benzamide | |
| 45 | pyrazine-2-carboxylic acid benzyl-[3-(4-isopropylamino- | 489.2 |
| | quinazolin-6-yl)-benzyl]-amide | |
| 46 | 6-bromo-pyridine-2-carboxylic acid benzyl-[3-(4-isopropylamino- | 566.2 |
| | quinazolin-6-yl)-benzyl]-amide | |
| 47 | N-benzyl-N-[3-(4-isopropylamino-quinazolin-6-yl)-benzyl]-2- | 517.3 |
| | phenoxy-acetamide | |
| 48 | N-benzyl-N-[3-(4-isopropylamino-quinazolin-6-yl)-benzyl]-2- | 502.3 |
| | pyridin-2-yl-acetamide | |
| 49 | N-benzyl-3-cyano-N-[3-(4-isopropylamino-quinazolin-6-yl)- | 512.2 |
| | benzyl]-benzamide | |
| 50 | thiophene-3-carboxylic acid benzyl-[3-(4-isopropylamino- | 493.2 |
| | quinazolin-6-yl)-benzyl]-amide | |
| 51 | N-benzyl-6-chloro-N-[3-(4-isopropylamino-quinazolin-6-yl)- | 522.2 |
| | benzyl]-nicotinamide | |
| 52 | 3-methyl-thiophene-2-carboxylic acid benzyl-[3-(4- | 507.2 |
| | isopropylamino-quinazolin-6-yl)-benzyl]-amide | |
| 53 | N-benzyl-2-cyano-N-[3-(4-isopropylamino-quinazolin-6-yl)- | 450.2 |
| | benzyl]-acetamide | |
| 54 | N-benzyl-N-[3-(4-isopropylamino-quinazolin-6-yl)-benzyl]-4- | 501.3 |
| | methyl-benzamide | |
| 55 | N-benzyl-N-[3-(4-isopropylamino-quinazolin-6-yl)-benzyl]-2- | 555.2 |
| | trifluoromethyl-benzamide | |
| 56 | N-benzyl-2-hydroxy-N-[3-(4-isopropylamino-quinazolin-6-yl)- | 503.2 |
| | benzyl]-benzamide | |
| 57 | N-[3-(4-diethylamino-quinazolin-6-yl)-benzyl]-N-methyl- | 425.2 |
| | benzamide | |
| 58 | 2-bromo-N-[3-(4-diethylamino-quinazolin-6-yl)-benzyl]-N-methyl- | 503.1 |
| | benzamide | |
| 59 | N-[3-(4-diethylamino-quinazolin-6-yl)-benzyl]-3,4-dimethoxy-N- | 485.3 |
| | methyl-benzamide | |
| 60 | N-[3-(4-diethylamino-quinazolin-6-yl)-benzyl]-2,4-difluoro-N- | 461.2 |
| | methyl-benzamide | |
| 61 | isoquinoline-3-carboxylic acid [3-(4-diethylamino-quinazolin-6- | 476.2 |
| | yl)-benzyl]-methyl-amide | |
| 62 | N-[3-(4-diethylamino-quinazolin-6-yl)-benzyl]-N-methyl-3-phenyl- | 451.2 |
| | acrylamide | |
| 63 | 4-chloro-N-[3-(4-diethylamino-quinazolin-6-yl)-benzyl]-N-methyl- | 459.2 |
| | benzamide | |
| 64 | 4-cyano-N-[3-(4-diethylamino-quinazolin-6-yl)-benzyl]-N-methyl- | 450.2 |
| | benzamide | |
| 65 | pyrazine-2-carboxylic acid [3-(4-diethylamino-quinazolin-6-yl)- | 427.2 |
| | benzyl]-methyl-amide | |
| 66 | 6-bromo-pyridine-2-carboxylic acid [3-(4-diethylamino-quinazolin- | 504.1 |
| | 6-yl)-benzyl]-methyl-amide | |
| 67 | N-[3-(4-diethylamino-quinazolin-6-yl)-benzyl]-N-methyl-2- | 455.2 |
| | phenoxy-acetamide | |
| 68 | N-[3-(4-diethylamino-quinazolin-6-yl)-benzyl]-N-methyl-2- | 440.2 |
| | pyridin-2-yl-acetamide | |
| 69 | 3-cyano-N-[3-(4-diethylamino-quinazolin-6-yl)-benzyl]-N-methyl- | 450.2 |
| | benzamide | |
| 70 | thiophene-3-carboxylic acid [3-(4-diethylamino-quinazolin-6-yl)- | 431.2 |
| | benzyl]-methyl-amide | |
| 71 | 6-chloro-N-[3-(4-diethylamino-quinazolin-6-yl)-benzyl]-N-methyl- | 460.2 |
| | nicotinamide | |
| 72 | 3-methyl-thiophene-2-carboxylic acid [3-(4-diethylamino- | 445.2 |
| | quinazolin-6-yl)-benzyl]-methyl-amide | |
| 73 | N-[3-(4-diethylamino-quinazolin-6-yl)-benzyl]-6-hydroxy-N- | 442.2 |
| | methyl-nicotinamide | |
| 74 | 2-cyano-N-[3-(4-diethylamino-quinazolin-6-yl)-benzyl]-N-methyl- | 388.2 |
| | acetamide | |
| 75 | N-[3-(4-diethylamino-quinazolin-6-yl)-benzyl]-4,N-dimethyl- | 439.2 |
| | benzamide | |
| 76 | N-[3-(4-diethylamino-quinazolin-6-yl)-benzyl]-N-methyl-2- | 493.2 |
| | trifluoromethyl-benzamide | |
| 77 | 5-bromo-thiophene-2-carboxylic acid [3-(4-diethylamino- | 509.1 |
| | quinazolin-6-yl)-benzyl]-methyl-amide | |
| 78 | N-[3-(4-diethylamino-quinazolin-6-yl)-benzyl]-N-methyl- | 426.2 |
| | isonicotinamide | |
| 79 | N-[3-(4-diethylamino-quinazolin-6-yl)-benzyl]-N-methyl- | 391.2 |
| | butyramide | |
| 80 | N-[3-(4-diethylamino-quinazolin-6-yl)-benzyl]-2-hydroxy-N- | 441.2 |
| | methyl-benzamide | |
| 81 | N-[3-(4-diethylamino-quinazolin-6-yl)-benzyl]-2-fluoro-N-methyl- | 511.2 |
| | 4-trifluoromethyl-benzamide | |
| 82 | thiophene-2-carboxylic acid [3-(4-diethylamino-quinazolin-6-yl)- | 431.2 |
| | benzyl]-methyl-amide | |
| 83 | N-[3-(4-diethylamino-quinazolin-6-yl)-benzyl]-N-methyl- | 426.2 |
| | nicotinamide | |
| 84 | N-[3-(4-diethylamino-quinazolin-6-yl)-benzyl]-2-dimethylamino- | 406.3 |
| | N-methyl-acetamide | |
| 85 | N-[3-(4-diethylamino-quinazolin-6-yl)-benzyl]-4-methoxy-N- | 455.2 |
| | methyl-benzamide | |
| 86 | 2-chloro-N-[3-(4-diethylamino-quinazolin-6-yl)-benzyl]-N-methyl- | 459.2 |
| | benzamide | |
| 87 | furan-2-carboxylic acid [3-(4-diethylamino-quinazolin-6-yl)- | 415.2 |
| | benzyl]-methyl-amide | |
| 88 | pyridine-2-carboxylic acid [3-(4-diethylamino-quinazolin-6-yl)- | 426.2 |
| | benzyl]-methyl-amide | |
| 89 | N-[3-(4-phenylamino-quinazolin-6-yl)-benzyl]-N-propyl- | 473.2 |
| | benzamide | |
| 90 | 2-phenoxy-N-[3-(4-phenylamino-quinazolin-6-yl)-benzyl]-N- | 503.2 |
| | propyl-acetamide | |
| 91 | 4-methyl-N-[3-(4-phenylamino-quinazolin-6-yl)-benzyl]-N-propyl- | 487.2 |
| | benzamide | |
| 92 | N-[3-(4-phenylamino-quinazolin-6-yl)-benzyl]-N-propyl- | 474.2 |
| | isonicotinamide | |
| 93 | 2-fluoro-N-[3-(4-phenylamino-quinazolin-6-yl)-benzyl]-N-propyl- | 559.2 |
| | 4-trifluoromethyl-benzamide | |
| 94 | 4-methoxy-N-[3-(4-phenylamino-quinazolin-6-yl)-benzyl]-N- | 503.2 |
| | propyl-benzamide | |
| 95 | pyridine-2-carboxylic acid [3-(4-phenylamino-quinazolin-6-yl)- | 474.2 |
| | benzyl]-propyl-amide | |
| 96 | 3-methyl-thiophene-2-carboxylic acid cyclopropyl-[3-(4-pyrrolidin- | 469.2 |
| | 1-yl-quinazolin-6-yl)-benzyl]-amide | |
| 97 | 2-bromo-N-[3-(4-phenylamino-quinazolin-6-yl)-benzyl]-N-propyl- | 551.1 |
| | benzamide | |
| 98 | 3,4-dimethoxy-N-[3-(4-phenylamino-quinazolin-6-yl)-benzyl]-N- | 533.3 |
| | propyl-benzamide | |
| 99 | 2,4-difluoro-N-[3-(4-phenylamino-quinazolin-6-yl)-benzyl]-N- | 509.2 |
| | propyl-benzamide | |
| 100 | isoquinoline-3-carboxylic acid [3-(4-phenylamino-quinazolin-6-yl)- | 524.2 |
| | benzyl]-propyl-amide | |
| 101 | 3-phenyl-N-[3-(4-phenylamino-quinazolin-6-yl)-benzyl]-N-propyl- | 499.2 |
| | acrylamide | |
| 102 | 4-chloro-N-[3-(4-phenylamino-quinazolin-6-yl)-benzyl]-N-propyl- | 507.2 |
| | benzamide | |
| 103 | 4-cyano-N-[3-(4-phenylamino-quinazolin-6-yl)-benzyl]-N-propyl- | 498.2 |
| | benzamide | |
| 104 | pyrazine-2-carboxylic acid [3-(4-phenylamino-quinazolin-6-yl)- | 475.2 |
| | benzyl]-propyl-amide | |
| 105 | 6-bromo-pyridine-2-carboxylic acid [3-(4-phenylamino-quinazolin- | 552.1 |
| | 6-yl)-benzyl]-propyl-amide | |
| 106 | N-[3-(4-phenylamino-quinazolin-6-yl)-benzyl]-N-propyl-2-pyridin- | 488.2 |
| | 2-yl-acetamide | |
| 107 | 3-cyano-N-[3-(4-phenylamino-quinazolin-6-yl)-benzyl]-N-propyl- | 498.2 |
| | benzamide | |
| 108 | thiophene-3-carboxylic acid [3-(4-phenylamino-quinazolin-6-yl)- | 479.2 |
| | benzyl]-propyl-amide | |
| 109 | 6-chloro-N-[3-(4-phenylamino-quinazolin-6-yl)-benzyl]-N-propyl- | 508.2 |
| | nicotinamide | |
| 110 | 3-methyl-thiophene-2-carboxylic acid [3-(4-phenylamino- | 493.2 |
| | quinazolin-6-yl)-benzyl]-propyl-amide | |
| 111 | 6-hydroxy-N-[3-(4-phenylamino-quinazolin-6-yl)-benzyl]-N- | 490.2 |
| | propyl-nicotinamide | |
| 112 | 2-cyano-N-[3-(4-phenylamino-quinazolin-6-yl)-benzyl]-N-propyl- | 436.2 |
| | acetamide | |
| 113 | N-[3-(4-phenylamino-quinazolin-6-yl)-benzyl]-N-propyl-2- | 541.2 |
| | trifluoromethyl-benzamide | |
| 114 | 5-bromo-thiophene-2-carboxylic acid [3-(4-phenylamino- | 557.1 |
| | quinazolin-6-yl)-benzyl]-propyl-amide | |
| 115 | N-[3-(4-phenylamino-quinazolin-6-yl)-benzyl]-N-propyl- | 439.2 |
| | butyramide | |
| 116 | thiophene-2-carboxylic acid [3-(4-phenylamino-quinazolin-6-yl)- | 479.2 |
| | benzyl]-propyl-amide | |
| 117 | N-[3-(4-phenylamino-quinazolin-6-yl)-benzyl]-N-propyl- | 474.2 |
| | nicotinamide | |
| 118 | 2-dimethylamino-N-[3-(4-phenylamino-quinazolin-6-yl)-benzyl]- | 454.3 |
| | N-propyl-acetamide | |
| 119 | 2-chloro-N-[3-(4-phenylamino-quinazolin-6-yl)-benzyl]-N-propyl- | 507.2 |
| | benzamide | |
| 120 | furan-2-carboxylic acid [3-(4-phenylamino-quinazolin-6-yl)- | 463.2 |
| | benzyl]-propyl-amide | |
| 121 | N-cyclopropyl-N-[3-(4-pyrrolidin-1-yl-quinazolin-6-yl)-benzyl]- | 449.2 |
| | benzamide | |
| 122 | 2-bromo-N-cyclopropyl-N-[3-(4-pyrrolidin-1-yl-quinazolin-6-yl)- | 527.1 |
| | benzyl]-benzamide | |
| 123 | N-cyclopropyl-3,4-dimethoxy-N-[3-(4-pyrrolidin-1-yl-quinazolin- | 509.3 |
| | 6-yl)-benzyl]-benzamide | |
| 124 | N-cyclopropyl-2,4-difluoro-N-[3-(4-pyrrolidin-1-yl-quinazolin-6- | 485.2 |
| | yl)-benzyl]-benzamide | |
| 125 | isoquinoline-3-carboxylic acid cyclopropyl-[3-(4-pyrrolidin-1-yl- | 500.2 |
| | quinazolin-6-yl)-benzyl]-amide | |
| 126 | N-cyclopropyl-3-phenyl-N-[3-(4-pyrrolidin-1-yl-quinazolin-6-yl)- | 475.2 |
| | benzyl]-acrylamide | |
| 127 | 4-chloro-N-cyclopropyl-N-[3-(4-pyrrolidin-1-yl-quinazolin-6-yl)- | 483.2 |
| | benzyl]-benzamide | |
| 128 | 4-cyano-N-cyclopropyl-N-[3-(4-pyrrolidin-1-yl-quinazolin-6-yl)- | 474.2 |
| | benzyl]-benzamide | |
| 129 | pyrazine-2-carboxylic acid cyclopropyl-[3-(4-pyrrolidin-1-yl- | 451.2 |
| | quinazolin-6-yl)-benzyl]-amide | |
| 130 | 6-bromo-pyridine-2-carboxylic acid cyclopropyl-[3-(4-pyrrolidin- | 528.1 |
| | 1-yl-quinazolin-6-yl)-benzyl]-amide | |
| 131 | thiophene-3-carboxylic acid cyclopropyl-[3-(4-pyrrolidin-1-yl- | 455.2 |
| | quinazolin-6-yl)-benzyl]-amide | |
| 132 | 6-chloro-N-cyclopropyl-N-[3-(4-pyrrolidin-1-yl-quinazolin-6-yl)- | 484.2 |
| | benzyl]-nicotinamide | |
| 133 | 2-cyano-N-cyclopropyl-N-[3-(4-pyrrolidin-1-yl-quinazolin-6-yl)- | 412.2 |
| | benzyl]-acetamide | |
| 134 | N-cyclopropyl-4-methyl-N-[3-(4-pyrrolidin-1-yl-quinazolin-6-yl)- | 463.2 |
| | benzyl]-benzamide | |
| 135 | N-cyclopropyl-N-[3-(4-pyrrolidin-1-yl-quinazolin-6-yl)-benzyl]-2- | 517.2 |
| | trifluoromethyl-benzamide | |
| 136 | 5-bromo-thiophene-2-carboxylic acid cyclopropyl-[3-(4-pyrrolidin- | 533.1 |
| | 1-yl-quinazolin-6-yl)-benzyl]-amide | |
| 137 | N-cyclopropyl-N-[3-(4-pyrrolidin-1-yl-quinazolin-6-yl)-benzyl]- | 450.2 |
| | isonicotinamide | |
| 138 | N-cyclopropyl-N-[3-(4-pyrrolidin-1-yl-quinazolin-6-yl)-benzyl]- | 415.2 |
| | butyramide | |
| 139 | N-cyclopropyl-2-fluoro-N-[3-(4-pyrrolidin-1-yl-quinazolin-6-yl)- | 535.2 |
| | benzyl]-4-trifluoromethyl-benzamide | |
| 140 | thiophene-2-carboxylic acid cyclopropyl-[3-(4-pyrrolidin-1-yl- | 455.2 |
| | quinazolin-6-yl)-benzyl]-amide | |
| 141 | N-cyclopropyl-N-[3-(4-pyrrolidin-1-yl-quinazolin-6-yl)-benzyl]- | 450.2 |
| | nicotinamide | |
| 142 | N-cyclopropyl-4-methoxy-N-[3-(4-pyrrolidin-1-yl-quinazolin-6- | 479.2 |
| | yl)-benzyl]-benzamide | |
| 143 | 2-chloro-N-cyclopropyl-N-[3-(4-pyrrolidin-1-yl-quinazolin-6-yl)- | 483.2 |
| | benzyl]-benzamide | |
| 144 | furan-2-carboxylic acid cyclopropyl-[3-(4-pyrrolidin-1-yl- | 439.2 |
| | quinazolin-6-yl)-benzyl]-amide | |
| 145 | N-cyclopropyl-2-phenoxy-N-[3-(4-pyrrolidin-1-yl-quinazolin-6- | 479.2 |
| | yl)-benzyl]-acetamide | |
| 146 | 3-cyano-N-cyclopropyl-N-[3-(4-pyrrolidin-1-yl-quinazolin-6-yl)- | 474.2 |
| | benzyl]-benzamid | |
| 147 | pyridine-2-carboxylic acid cyclopropyl-[3-(4-pyrrolidin-1-yl- | 450.2 |
| | quinazolin-6-yl)-benzyl]-amide | |
| 148 | N-benzyl-N-[3-(4-isopropylamino-quinazolin-6-yl)-benzyl]- | 453.3 |
| | butyramide | |
| 149 | furan-2-carboxylic acid benzyl-[3-(4-isopropylamino-quinazolin-6- | 477.2 |
| | yl)-benzyl]-amide | |
| 150 | N-benzyl-2-chloro-N-[3-(4-isopropylamino-quinazolin-6-yl)- | 521.2 |
| | benzyl]-benzamide | |
| 151 | N-benzyl-N-[3-(4-isopropylamino-quinazolin-6-yl)-benzyl]- | 488.2 |
| | isonicotinamide | |
| 152 | N-benzyl-N-[3-(4-cyclopropylamino-quinazolin-6-yl)-benzyl]-2- | 571.2 |
| | fluoro-4-trifluoromethyl-benzamide | |
| 153 | N-benzyl-N-[3-(4-isopropylamino-quinazolin-6-yl)-benzyl]- | 488.2 |
| | nicotinamide | |
| 154 | thiophene-2-carboxylic acid benzyl-[3-(4-isopropylamino- | 493.2 |
| | quinazolin-6-yl)-benzyl]-amide | |
| 155 | N-benzyl-N-[3-(4-isopropylamino-quinazolin-6-yl)benzyl]-4- | 517.3 |
| | methoxy-benzamide | |
| 156 | N-cyclopropyl-N-[3-(4-cyclopropylamino-quinazolin-6-yl)- | 451.2 |
| | benzyl]-2-hydroxy-benzamide | |
| 157 | 2-bromo-N-cyclopropyl-N-[3-(4-cyclopropylamino-quinazolin-6- | 513.1 |
| | yl)-benzyl]-benzamide | |
| 158 | N-cyclopropyl-N-[3-(4-cyclopropylamino-quinazolin-6-yl)- | 495.2 |
| | benzyl]-3,4-dimethoxy-benzamide | |
| 159 | isoquinoline-3-carboxylic acid cyclopropyl-[3-(4- | 486.2 |
| | cyclopropylamino-quinazolin-6-yl)-benzyl]-amide | |
| 160 | 4-chloro-N-cyclopropyl-N-[3-(4-cyclopropylamino-quinazolin-6- | 469.2 |
| | yl)-benzyl]-benzamide | |
| 161 | 4-cyano-N-cyclopropyl-N-[3-(4-cyclopropylamino-quinazolin-6- | 460.2 |
| | yl)-benzyl]-benzamide | |
| 162 | pyrazine-2-carboxylic acid cyclopropyl-[3-(4-cyclopropylamino- | 437.2 |
| | quinazolin-6-yl)-benzyl]-amide | |
| 163 | 6-bromo-pyridine-2-carboxylic acid cyclopropyl-[3-(4- | 514.1 |
| | cyclopropylamino-quinazolin-6-yl)-benzyl]-amide | |
| 164 | N-cyclopropyl-N-[3-(4-cyclopropylamino-quinazolin-6-yl)- | 465.2 |
| | benzyl]-2-phenoxy-acetamide | |
| 165 | N-cyclopropyl-N-[3-(4-cyclopropylamino-quinazolin-6-yl)- | 436.2 |
| | benzyl]-isonicotinamide | |
| 166 | 5-bromo-thiophene-2-carboxylic acid cyclopropyl-[3-(4- | 519.1 |
| | cyclopropylamino-quinazolin-6-yl)-benzyl]-amide | |
| 167 | N-cyclopropyl-N-[3-(4-cyclopropylamino-quinazolin-6-yl)- | 503.2 |
| | benzyl]-2-trifluoromethyl-benzamide | |
| 168 | N-cyclopropyl-N-[3-(4-cyclopropylamino-quinazolin-6-yl)- | 401.2 |
| | benzyl]-butyramide | |
| 169 | furan-2-carboxylic acid cyclopropyl-[3-(4-cyclopropylamino- | 425.2 |
| | quinazolin-6-yl)-benzyl]-amide | |
| 170 | pyridine-2-carboxylic acid cyclopropyl-[3-(4-cyclopropylamino- | 436.2 |
| | quinazolin-6-yl)-benzyl]-amide | |
| 171 | N-cyclopropyl-N-[3-(4-cyclopropylamino-quinazolin-6-yl)- | 465.2 |
| | benzyl]-4-methoxy-benzamide | |
| 172 | 2-chloro-N-cyclopropyl-N-[3-(4-cyclopropylamino-quinazolin-6- | 469.2 |
| | yl)-benzyl]-benzamide | |
| 173 | N-cyclopropyl-N-[3-(4-cyclopropylamino-quinazolin-6-yl)- | 416.2 |
| | benzyl]-2-dimethylamino-acetamide | |
| 174 | N-cyclopropyl-N-[3-(4-cyclopropylamino-quinazolin-6-yl)- | 450.2 |
| | benzyl]-2-pyridin-2-yl-acetamide | |
| 175 | N-[3-(4-cyclopropylamino-quinazolin-6-yl)-benzyl]-4,N-dimethyl- | 423.2 |
| | benzamide | |
| 176 | 3-cyano-N-cyclopropyl-N-[3-(4-cyclopropylamino-quinazolin-6- | 460.2 |
| | yl)-benzyl]-benzamide | |
| 177 | N-cyclopropyl-N-[3-(4-cyclopropylamino-quinazolin-6-yl)- | 521.2 |
| | benzyl]-2-fluoro-4-trifluoromethyl-benzamide | |
| 178 | thiophene-2-carboxylic acid cyclopropyl-[3-(4-cyclopropylamino- | 441.2 |
| | quinazolin-6-yl)-benzyl]-amide | |
| 179 | 6-chloro-N-cyclopropyl-N-[3-(4-cyclopropylamino-quinazolin-6- | 470.2 |
| | yl)-benzyl]-nicotinamide | |
| 180 | 1-[3-(4-cyclopropylamino-2-methyl-quinazolin-6-yl)-benzyl]- | 387.2 |
| | pyrrolidine-2,5-dione | |
| 181 | 1-[3-(4-cyclopropylamino-2-trifluoromethyl-quinazolin-6-yl)- | 441.2 |
| | benzyl]-pyrrolidine-2,5-dione | |
| 182 | 1-[3-(2,4-bis-cyclopropylamino-quinazolin-6-yl)-benzyl]- | 428.2 |
| | pyrrolidine-2,5-dione | |
| 183 | 1-[3-(2-amino-4-cyclopropylamino-quinazolin-6-yl)-benzyl]- | 388.2 |
| | pyrrolidine-2,5-dione | |
| 184 | 1-[3-(4-cyclopropylamino-2-methylamino-quinazolin-6-yl)- | 402.2 |
| | benzyl]-pyrrolidine-2,5-dione | |
| 185 | 1-[3-(4-cyclopropylamino-2-phenylamino-quinazolin-6-yl)- | 464.2 |
| | benzyl]-pyrrolidine-2,5-dione | |
| 186 | 1-[3-(4-cyclopropylamino-2-dimethylamino-quinazolin-6-yl)- | 416.2 |
| | benzyl]-pyrrolidine-2,5-dione | |
| 187 | 1-[3-(2-tert-butyl-4-cyclopropylamino-quinazolin-6-yl)-benzyl]- | 429.2 |
| | pyrrolidine-2,5-dione | |
| 188 | N-tert-butyl-2-cyano-N-(5-(4-(cyclopropylamino)quinazolin-6-yl)- | 432.2 |
| | 2-fluorobenzyl)acetamide | |
| 189 | 4-(5-(4-(cyclopropylamino)quinazolin-6-yl)-2- | 407.2 |
| | fluorobenzyl)morpholine-3,5-dione | |
| 190 | methyl 2-(2-cyano-N-(3-(4-(cyclopropylamino)quinazolin-6- | 430.2 |
| | yl)benzyl)acetamido)acetate | |
| 191 | N-cyclopropyl-N-(3-(4-(cyclopropylamino)quinazolin-6-yl)-2- | 391.2 |
| | fluorobenzyl)acetamide | |
| 192 | 1-(5-(4-(cyclopropylamino)quinazolin-6-yl)-2-fluorobenzyl)-1H- | 389.1 |
| | pyrrole-2,5-dione | |
| 193 | 1-(5-(4-(cyclopropylamino)quinazolin-6-yl)-2- | 391.2 |
| | fluorobenzyl)pyrrolidine-2,5-dione | |
| 194 | 1-[3-(4-cyclopropylamino-quinazolin-6-yl)-benzyl]-piperidine-2,6- | |
| | dione | |
| 195 | 2-cyano-N-[3-(4-cyclopropylamino-quinazolin-6-yl)-benzyl]-N- | 435.2 |
| | pyridin-3-yl-acetamid | |
| 196 | 1-[3-(4-tert-butylamino-quinazolin-6-yl)-benzyl]-pyrrolidine-2,5- | 389.2 |
| | dione; hydrochloride | |
| 197 | 2-cyano-N-cyclopropyl-N-[3-(4-cyclopropylamino-quinazolin-6- | 416.2 |
| | yl)-2-fluoro-benzyl]-acetamide | |
| 198 | 1-[3-(4-methylamino-quinazolin-6-yl)-benzyl]-pyrrolidine-2,5- | 347.2 |
| | dione | |
| 199 | 1-(3-(4-(benzylamino)quinazolin-6-yl)benzyl)pyrrolidine-2,5-dione | 423.2 |
| 200 | N-cyanomethyl-N-[3-(4-cyclopropylamino-quinazolin-6-yl)- | 372.2 |
| | benzyl]-acetamide | |
| 201 | N-[5-(4-tert-butylamino-quinazolin-6-yl)-2-fluoro-benzyl]-2- | 432.2 |
| | cyano-N-cyclopropyl-acetamide | |
| 202 | N-tert-butyl-N-[3-(4-cyclopropylamino-quinazolin-6-yl)-benzyl]- | 389.2 |
| | acetamide | |
| 203 | {acetyl-[3-(4-cyclopropylamino-quinazolin-6-yl)-benzyl]-amino}- | 405.2 |
| | methyl acetate | |
| 204 | tert-butyl 2-(N-(3-(4-(cyclopropylamino)quinazolin-6- | 476.3 |
| | yl)benzyl)acetamido)ethyl carbamate | |
| 205 | N-cyclopropyl-N-[3-(4-cyclopropylamino-quinazolin-6-yl)- | 441.2 |
| | benzyl]-3,3,3-trifluoro-propionamide; hydrochloride | |
| 206 | cyclopropyl-{6-[3-(1,1-dioxo-116-isothiazolidin-2-ylmethyl)- | |
| | phenyl]-quinazolin-4-yl}-amine; hydrochloride | |
| 207 | N-(3-(4-(cyclopropylamino)quinazolin-6-yl)benzyl)-N- | 409.2 |
| | phenylacetamide hydrochloride | |
| 208 | N-cyclopropyl-N-[3-(4-cyclopropylamino-quinazolin-6-yl)- | 391.2 |
| | benzyl]-2-fluoro-acetamide | |
| 209 | N-cyclopentyl-N-(3-(4-(cyclopropylamino)quinazolin-6- | 401.2 |
| | yl)benzyl)acetamide | |
| 210 | N-cyclopentyl-N-(3-(4-(cyclopropylamino)quinazolin-6- | 437.2 |
| | yl)benzyl)methane sulfonamide | |
| 211 | 2-cyano-N-cyclopropyl-N-[3-(4-cyclopropylamino-quinazolin-6- | 398.2 |
| | yl)-benzyl]-acetamide | |
| 212 | 4-(3-(4-(cyclopropylamino)quinazolin-6-yl)benzyl)morpholine-3,5- | 389.2 |
| | dione | |
| 213 | N-(3-(4-(cyclopropylamino)quinazolin-6-yl)benzyl)-N-(2- | 391.2 |
| | methoxyethyl)acetamide | |
| 214 | N-[3-(4-cyclopropylamino-quinazolin-6-yl)-benzyl]-N-phenethyl- | 437.2 |
| | acetamide | |
| 215 | N-(3-(4-(cyclopropylamino)quinazolin-6-yl)benzyl)-N-(furan-2-yl- | 449.2 |
| | methyl)methane sulfonamide | |
| 216 | N-[3-(4-cyclopropylamino-quinazolin-6-yl)-benzyl]-N-furan-2-yl- | 413.2 |
| | methyl-acetamide | |
| 217 | 1-[3-(4-cyclopropylamino-quinazolin-6-yl)-benzyl]-pyrrolidin-2- | 359.2 |
| | one; hydrochloride | |
| 218 | N-butyl-N-[3-(4-cyclopropylamino-quinazolin-6-yl)-benzyl]- | 389.2 |
| | acetamide; hydrochloride | |
| 219 | N-benzyl-N-[3-(4-cyclopropylamino-quinazolin-6-yl)-benzyl]- | 423.2 |
| | acetamide; hydrochloride | |
| 220 | 1-[3-(4-cyclopropylamino-quinazolin-6-yl)-benzyl]-3-phenyl- | 463.2 |
| | piperidine-2,6-dione; hydrochloride | |
| 221 | N-[3-(4-cyclopropylamino-quinazolin-6-yl)-benzyl]-N-methyl- | 347.2 |
| | acetamide | |
| 222 | N-benzyl-N-(3-(4-(cyclopropylamino)quinazolin-6- | 459.2 |
| | yl)benzyl)methane sulfonamide | |
| 223 | N-(3-(4-(cyclopropylamino)quinazolin-6-yl)benzyl)-N-methyl | 383.2 |
| | methane sulfonamide | |
| 224 | 1-[3-(4-cyclopropylamino-quinazolin-6-yl)-benzyl]-4-methyl- | |
| | piperidine-2,6-dione; hydrochloride | |
| 225 | N-cyclopropyl-N-(3-(4-(cyclopropylamino)quinazolin-6- | 477.2 |
| | yl)benzyl)-2,2,2-trifluorethane sulfonamide | |
| 226 | N-cyclopropyl-N-(3-(4-(cyclopropylamino)quinazolin-6- | 539.2 |
| | yl)benzyl)-4-(trifluoromethyl)benzole sulfonamide | |
| 227 | N-cyclopropyl-N-(3-(4-(cyclopropylamino)quinazolin-6- | 471.2 |
| | yl)benzyl)benzole sulfonamide | |
| 228 | 2-[3-(4-cyclopropylamino-quinazolin-6-yl)-benzyl]-hexahydro- | |
| | isoindole-1,3-dione; hydrochloride | |
| 229 | 1-[3-(4-cyclopropylamino-quinazolin-6-yl)-benzyl]-3-phenyl- | |
| | pyrrolidine-2,5-dione; hydrochloride | |
| 230 | N-cyclopropyl-N-[5-(4-cyclopropylamino-quinazolin-6-yl)-pyridin- | 436.2 |
| | 3-yl-methyl]-benzamide; hydrochloride | |
| 231 | N-cyclopropyl-N-(1-(3-(4-(cyclopropylamino)quinazolin-6- | 423.2 |
| | yl)phenyl)ethyl)methane sulfonamide | |
| 232 | N-cyclopropyl-N-[5-(4-cyclopropylamino-quinazolin-6-yl)-furan- | 363.2 |
| | 2-yl-methyl]-acetamide | |
| 233 | N-cyclopropyl-N-{1-[3-(4-cyclopropylamino-quinazolin-6-yl)- | 449.2 |
| | phenyl]-ethyl}-benzamide | |
| 234 | N-cyclopropyl-N-[5-(4-cyclopropylamino-quinazolin-6-yl)-pyridin- | |
| | 3-yl-methyl]-methane sulfonamide | |
| 235 | N-cyclopropyl-N-{1-[3-(4-cyclopropylamino-quinazolin-6-yl)- | 387.2 |
| | phenyl]-ethyl}-acetamide | |
| 236 | N-{1-[3-(4-cyclopropylamino-quinazolin-6-yl)-benzyl]-2,5-dioxo- | 484.2 |
| | pyrrolidin-3-yl}-2,2,2-trifluoro-acetamide; hydrochloride | |
| 237 | N-cyclopropyl-N-[3-(4-cyclopropylamino-quinazolin-6-yl)- | 431.2 |
| | benzyl]-malonic acid methyl ester | |
| 238 | N-cyclopropyl-N-[3-(4-cyclopropylamino-quinazolin-6-yl)- | 417.2 |
| | benzyl]-oxalic acid methyl ester | |
| 239 | N-cyclopropyl-N-[3-(4-cyclopropylamino-quinazolin-6-yl)-5- | 387.2 |
| | methyl-benzyl]-acetamide | |
| 240 | N-cyclopropyl-N-[5-(4-cyclopropylamino-quinazolin-6-yl)-pyridin- | |
| | 3-yl-methyl]-acetamide | |
| 241 | 2-[3-(4-cyclopropylamino-quinazolin-6-yl)-benzyl]-isoindole-1,3- | 421.2 |
| | dione; hydrochloride | |
| 242 | 2-[3-(4-cyclopropylamino-quinazolin-6-yl)-benzyl]-hexahydro- | 427.2 |
| | isoindole-1,3-dione; hydrochloride | |
| 243 | 8-[3-(4-cyclopropylamino-quinazolin-6-yl)-benzyl]-8-aza- | 441.2 |
| | Spiro[4.5]decane-7,9-dione; hydrochloride | |
| 244 | N-cyclopropyl-N-[3-(4-cyclopropylamino-quinazolin-6-yl)-2,6- | 471.2 |
| | difluoro-benzyl]-benzamide | |
| 245 | 1-[3-(4-cyclopropylamino-quinazolin-6-yl)-benzyl]-3-methyl- | 387.2 |
| | pyrrolidine-2,5-dione | |
| 246 | N-cyclopropyl-N-(3-(4-(cyclopropylamino)quinazolin-6-yl)-2,6- | 445.2 |
| | difluorobenzyl)methane sulfonamide | |
| 247 | 1-cyclopropyl-1-[3-(4-cyclopropylamino-quinazolin-6-yl)-benzyl]- | 416.2 |
| | 3-isopropyl urea | |
| 248 | N-cyclopropyl-N-[3-(4-cyclopropylamino-quinazolin-6-yl)- | 436.2 |
| | benzyl]-nicotinamide | |
| 249 | N-cyclopropyl-N-[3-(4-cyclopropylamino-quinazolin-6-yl)-2,6- | 409.2 |
| | difluoro-benzyl]-acetamide | |
| 250 | N-cyclopropyl-N-(3-(4-(cyclopropylamino)quinazolin-6-yl)-4- | 439.2 |
| | methoxybenzyl)methane sulfonamide | |
| 251 | N-cyclopropyl-N-(5-(4-(cyclopropylamino)quinazolin-6-yl)-2- | 427.2 |
| | fluorobenzyl)methane sulfonamide | |
| 252 | N-cyclopropyl-N-[3-(4-cyclopropylamino-quinazolin-6-yl)- | 403.2 |
| | benzyl]-2-methoxy-acetamide | |
| 253 | isoxazole-5-carboxylic acid cyclopropyl-[3-(4-cyclopropylamino- | 426.2 |
| | quinazolin-6-yl)-benzyl]-amide | |
| 254 | N-cyclopropyl-N-[3-(4-cyclopropylamino-quinazolin-6-yl)- | 525.2 |
| | benzyl]-2,3,4,5,6-pentafluoro-benzamide | |
| 255 | N-cyclopropyl-N-[3-(4-cyclopropylamino-quinazolin-6-yl)- | 461.2 |
| | benzyl]-3-phenyl-acrylamide | |
| 256 | pentanoic acid cyclopropyl-[3-(4-cyclopropylamino-quinazolin-6- | 415.2 |
| | yl)-benzyl]-amide | |
| 258 | 1-[3-(4-cyclopropylamino-quinazolin-6-yl)-benzyl]-pyrrolidine- | 373.2 |
| | 2,5-dione | |
| 259 | N-cyclopropyl-N-[5-(4-cyclopropylamino-quinazolin-6-yl)-2- | 403.2 |
| | methoxy-benzyl]-acetamide | |
| 260 | N-cyclopropyl-N-[5-(4-cyclopropylamino-quinazolin-6-yl)-2- | 391.2 |
| | fluoro-benzyl]-acetamide | |
| 261 | N-cyclopropyl-N-[3-(4-cyclopropylamino-quinazolin-6-yl)-4- | 403.2 |
| | methoxy-benzyl]-acetamide | |
| 262 | N-cyclopropyl-N-[4-(4-cyclopropylamino-quinazolin-6-yl)- | 379.2 |
| | thiophen-2-ylmethyl]-acetamide | |
| 263 | N-cyclopropyl-N-[3-(4-cyclopropylamino-quinazolin-6-yl)- | 463.2 |
| | benzyl]-3-phenyl-propionamide | |
| 264 | N-cyclopropyl-N-[3-(4-cyclopropylamino-quinazolin-6-yl)- | 449.2 |
| | benzyl]-2-phenyl-acetamide | |
| 265 | N-cyclopropyl-N-(3-(4-(cyclopropylamino)quinazolin-6- | 441.3 |
| | yl)benzyl)cyclohexane carboxamide | |
| 266 | N-cyclopropyl-N-(3-(4-(cyclopropylamino)quinazolin-6- | 415.2 |
| | yl)benzyl)pivalamide | |
| 267 | N-cyclopropyl-N-(3-(4-(cyclopropylamino)quinazolin-6-yl)-4- | 465.2 |
| | methoxybenzyl)benzamide hydrochloride | |
| 268 | N-cyclopropyl-N-(3-(4-(cyclopropylamino)quinazolin-6-yl)-4- | 465.2 |
| | methoxybenzyl)benzamide | |
| 269 | N-cyclopropyl-N-(5-(4-(cyclopropylamino)quinazolin-6-yl)-2- | 453.2 |
| | fluorobenzyl)benzamide | |
| 270 | N-cyclopropyl-N-(3-(4-(cyclopropylamino)quinazolin-6- | 409.2 |
| | yl)benzyl)methane sulfonamide | |
| 271 | N-cyclopropyl-N-(3-(4-(cyclopropylamino)quinazolin-6- | 373.2 |
| | yl)benzyl)acetamide | |
| 272 | N-cyclopropyl-N-(3-(4-(cyclopropyl(methyl)amino)quinazolin-6- | 450.2 |
| | yl)benzyl)picolinamide | |
| 273 | N-cyclopropyl-N-(3-(4-(cyclopropylamino)quinazolin-6- | 427.2 |
| | yl)benzyl)-2,2,2-trifluoroacetamide | |
| 274 | N-cyclopropyl-N-(3-(4-(cyclopropylamino)quinazolin-6- | 435.2 |
| | yl)benzyl)benzamide | |
| 276 | 1-(3-(4-(benzylamino)quinazolin-6-yl)benzyl)pyrrolidine-2,5-dione | 423.2 |
| 277 | 2-cyano-N-cyclopropyl-N-(5-(4-(cyclopropylamino)quinazolin-6- | 416.2 |
| | yl)-2-fluorobenzyl)acetamide | |
| 278 | 1-(5-(4-(cyclopropylamino)quinazolin-6-yl)-2- | 405.2 |
| | fluorobenzyl)piperidine-2,6-dione | |
| 279 | N-tert-butyl-N-(5-(4-(cyclopropylamino)quinazolin-6-yl)-2- | 407.2 |
| | fluorobenzyl)acetamide | |
| 280 | tert-butyl 2-(2-cyano-N-(3-(4-(cyclopropylamino)quinazolin-6- | 501.3 |
| | yl)benzyl)acetamido)ethyl carbamate | |
| 281 | 2-cyano-N-cyclopropyl-N-(5-(4-(cyclopropylamino)quinazolin-6- | 416.2 |
| | yl)-2-fluorobenzyl)acetamide hydrochloride | |
| 282 | N-cyclopropyl-N-(5-(4-(cyclopropylamino)quinazolin-6-yl)-2- | 459.2 |
| | fluorobenzyl)-3,3,3-trifluoropropanamide | |
| 283 | 2-chloro-N-cyclopropyl-N-(5-(4-(cyclopropylamino)quinazolin-6- | 425.2 |
| | yl)-2-fluorobenzyl)acetamide | |
| 284 | N-(2-aminoethyl)-2-cyano-N-(3-(4-(cyclopropylamino)quinazolin- | 401.2 |
| | 6-yl)benzyl)acetamide hydrochloride | |
| 285 | 1-(3-(4-(dimethylamino)quinazolin-6-yl)benzyl)pyrrolidine-2,5- | 361.2 |
| | dione hydrochloride | |
| 286 | 1-(3-(4-(butylamino)quinazolin-6-yl)benzyl)pyrrolidine-2,5-dione | 389.2 |
| | hydrochloride | |
| 287 | 1-(3-(4-(cyclopentylamino)quinazolin-6-yl)benzyl)pyrrolidine-2,5- | 401.2 |
| | dione hydrochloride | |
| 288 | 1-(3-(2-benzyl-4-(cyclopropylamino)quinazolin-6- | 463.2 |
| | yl)benzyl)pyrrolidine-2,5-dione | |
| 289 | 1-(3-(4-(cyclopropylamino)-2-(pyridin-2-yl)quinazolin-6- | 450.2 |
| | yl)benzyl)pyrrolidine-2,5-dione | |
| 290 | 1-(3-(4-(cyclopropylamino)-2-(pyridin-2-yl)quinazolin-6- | 458.2 |
| | yl)benzyl)pyrrolidine-2,5-dione | |
| 291 | 1-(3-(2-(benzylamino)-4-(cyclopropylamino)quinazolin-6- | 478.2 |
| | yl)benzyl)pyrrolidine-2,5-dione | |
| 292 | N-cyclopropyl-6-(3-(indolin-1-ylmethyl)phenyl)quinazoline-4- | |
| | amine | |
| 293 | 1-(3-(4-(cyclopropylamino)-2-phenylquinazolin-6- | 449.2 |
| | yl)benzyl)pyrrolidine-2,5-dione | |
| 294 | N-cyclopropyl-6-(3-((cyclopropylamino)methyl)- | 331.2 |
| | phenyl)quinazoline-4-amine | |
| 295 | tert-butyl 3-(4-(cyclopropylamino)quinazolin-6-yl)benzylcarbamate | 391.2 |
| 296 | N-cyclopropyl-6-(3-((dimethylamino)methyl)phenyl)quinazoline-4- | |
| | amine | |
| 297 | 6-(3-(aminomethyl)phenyl)-N-cyclopropylquinazoline-4-amine | |
| | dihydrochloride | |
| 298 | N-cyclopropyl-6-(3-((cyclopropylamino)methyl)-4- | 361.2 |
| | methoxyphenyl)quinazoline-4-amine | |
| 299 | N-cyclopropyl-6-(3-((cyclopropylamino)methyl)-4- | 349.2 |
| | fluorophenyl)quinazoline-4-amine | |
| 300 | N-cyclopropyl-6-(5-((cyclopropylamino)methyl)thiophen-3- | 337.1 |
| | yl)quinazoline-4-amine | |
| 301 | N-cyclopropyl-6-(5-((cyclopropylamino)methyl)-2- | 361.2 |
| | methoxyphenyl)quinazoline-4-amine | |
| 302 | N-(3-(4-(cyclopropylamino)quinazolin-6-yl)benzyl)acetamide | 333.2 |
| 303 | N-(3-(4-(cyclopropylamino)quinazolin-6-yl)benzyl)benzamide | 395.2 |
| 304 | N-cyclopropyl-6-(3-((cyclopropylamino)methyl)-2,4- | 367.2 |
| | difluorophenyl)quinazoline-4-amine | |
| 305 | N-cyclopropyl-6-(5-((cyclopropylamino)methyl)pyridin-3- | 332.2 |
| | yl)quinazoline-4-amine | |
| 306 | N-cyclopropyl-6-(3-((cyclopropylamino)methyl)-5- | |
| | methylphenyl)quinazoline-4-amine | |
| 307 | N-cyclopropyl-6-(3-(1-(cyclopropylamino)ethyl)phenyl)- | 345.2 |
| | quinazoline-4-amine | |
| 308 | 6-(3-((benzylamino)methyl)phenyl)-N-cyclopropylquinazoline-4- | |
| | amine | |
| 309 | N-cyclopropyl-6-(3-((methylamino)methyl)phenyl)quinazoline-4- | 305.2 |
| | amine | |
| 310 | 6-(3-((butylamino)methyl)phenyl)-N-cyclopropylquinazoline-4- | 347.2 |
| | amine | |
| 311 | N-(6-(3-(acetamidomethyl)phenyl)quinazolin-4-yl)-N-cyclopropyl | |
| | acetamide hydrochloride | |
| 312 | N-cyclopropyl-6-(3-((furan-2-ylmethylamino)methyl)phenyl)- | 371.2 |
| | quinazoline-4-amine dihydrochloride | |
| 313 | N-cyclopropyl-6-(3-((phenethylamino)methyl)phenyl)quinazoline- | 395.2 |
| | 4-amine | |
| 314 | N-cyclopropyl-6-(3-((2-methoxyethylamino)methyl)phenyl)- | 349.2 |
| | quinazoline-4-amine dihydrochloride | |
| 315 | 6-(3-((2-chloroethylamino)methyl)phenyl)-N- | |
| | cyclopropylquinazoline-4-amine | |
| 316 | 3-chloro-N-(3-(4-(cyclopropylamino)quinazolin-6- | 431.1 |
| | yl)benzyl)propane-1-sulfonamide hydrochloride | |
| 317 | 5-chloro-N-(3-(4-(cyclopropylamino)quinazolin-6- | 409.2 |
| | yl)benzyl)pentanamide hydrochloride | |
| 318 | 6-(3-((cyclopentylamino)methyl)phenyl)-N- | 359.2 |
| | cyclopropylquinazoline-4-amine | |
| 319 | N-cyclopropyl-6-(3-((phenylamino)methyl)phenyl)quinazoline-4- | 367.2 |
| | amine | |
| 320 | 2-(3-(4-(cyclopropylamino)quinazolin-6- | 330.2 |
| | yl)benzylamino)acetonitrile | |
| 321 | methyl 2-(3-(4-(cyclopropylamino)quinazolin-6- | 363.2 |
| | yl)benzylamino)acetate | |
| 322 | N-tert-butyl-6-(3-((cyclopropylamino)methyl)-4-fluorophenyl) | 365.2 |
| | quinazoline-4-amine | |
| 323 | 6-(3-((tert-butylamino)methyl)phenyl)-N-cyclopropylquinazoline- | 347.2 |
| | 4-amine | |
| 324 | N-cyclopropyl-6-(3-((cyclopropylamino)methyl)-2- | 349.2 |
| | fluorophenyl)quinazoline-4-amine | |
| 325 | N-cyclopropyl-6-(3-((pyridin-3- | 368.2 |
| | ylamino)methyl)phenyl)quinazoline-4-amine | |
| 326 | tert-butyl 2-(3-(4-(cyclopropylamino)quinazolin-6- | 434.3 |
| | yl)benzylamino)ethyl carbamate | |
| 327 | 6-(3-((tert-butylamino)methyl)-4-fluorophenyl)-N- | 365.2 |
| | cyclopropylquinazoline-4-amine | |
| 328 | 2-(2-(3-(4-(cyclopropylamino)quinazolin-6-yl)benzylamino)-2- | 453.2 |
| | oxoethyl) benzoic acid | |
| 329 | 4-oxo-4-(3-(4-(phenylamino)quinazolin-6-yl)benzylamino)butyric | 427.2 |
| | acid | |
| 330 | 4-(3-(4-(carboxymethylamino)quinazolin-6-yl)benzylamino)-4- | 409.2 |
| | oxobutyric acid | |
| 331 | N-benzyl-5-bromo-N-(3-(4-(isopropylamino)quinazolin-6- | 571.1 |
| | yl)benzyl)thiophene-2-carboxamide | |
| 332 | N-benzyl-N-(3-(4-(isopropylamino)quinazolin-6- | 488.2 |
| | yl)benzyl)picolinamide | |
| 333 | N-(3-(4-(cyclopropylamino)quinazolin-6-yl)benzyl)picolinamide | 396.2 |
| 334 | 2-chloro-N-(3-(4-(cyclopropylamino)quinazolin-6- | 429.1 |
| | yl)benzyl)benzamide | |
| 335 | N-(3-(4-(cyclopropylamino)quinazolin-6-yl)benzyl)-4- | 425.2 |
| | methoxybenzamide | |
| 336 | N-(3-(4-(cyclopropylamino)quinazolin-6-yl)benzyl)thiophene-2- | 401.1 |
| | carboxamide | |
| 337 | N-(3-(4-(cyclopropylamino)quinazolin-6-yl)benzyl)-2-fluoro-4- | 481.2 |
| | (trifluoromethyl)benzamide | |
| 338 | N-(3-(4-(cyclopropylamino)quinazolin-6-yl)benzyl)butyramide | 361.2 |
| 339 | 5-bromo-N-(3-(4-(cyclopropylamino)quinazolin-6- | 479.1 |
| | yl)benzyl)thiophene-2-carboxamide | |
| 340 | N-(3-(4-(cyclopropylamino)quinazolin-6-yl)benzyl)-2- | 463.2 |
| | (trifluoromethyl) benzamide | |
| 341 | N-(3-(4-(cyclopropylamino)quinazolin-6-yl)benzyl)-4- | 409.2 |
| | methylbenzamide | |
| 342 | 6-chloro-N-(3-(4-(cyclopropylamino)quinazolin-6- | 430.1 |
| | yl)benzyl)nicotinamide | |
| 343 | N-(3-(4-(cyclopropylamino)quinazolin-6-yl)benzyl)thiophene-3- | 401.1 |
| | carboxamide | |
| 344 | N-(3-(4-(cyclopropylamino)quinazolin-6-yl)benzyl)-2- | 425.2 |
| | phenoxyacetamide | |
| 345 | 6-bromo-N-(3-(4-(cyclopropylamino)quinazolin-6- | 474.1 |
| | yl)benzyl)picolinamide | |
| 346 | 4-cyano-N-(3-(4-(cyclopropylamino)quinazolin-6- | 420.2 |
| | yl)benzyl)benzamide | |
| 347 | N-(3-(4-(cyclopropylamino)quinazolin-6-yl)benzyl)cinnamamide | 421.2 |
| 348 | N-(3-(4-(cyclopropylamino)quinazolin-6-yl)benzyl)isoquinoline-3- | 446.2 |
| | carboxamide | |
| 349 | N-(3-(4-(cyclopropylamino)quinazolin-6-yl)benzyl)-2,4- | 431.2 |
| | difluorobenzamide | |
| 350 | 2-bromo-N-(3-(4-(cyclopropylamino)quinazolin-6- | 473.1 |
| | yl)benzyl)benzamide | |
| 351 | N-(3-(4-(cyclopropylamino)quinazolin-6-yl)benzyl)furan-2- | 385.2 |
| | carboxamide | |
| 352 | N-(3-(4-(cyclopropylamino)quinazolin-6-yl)benzyl)-2- | 376.2 |
| | (dimethylamino) acetamide | |
| 353 | N-(3-(4-(cyclopropylamino)quinazolin-6-yl)benzyl)nicotinamide | 396.2 |
| 354 | N-(3-(4-(cyclopropylamino)quinazolin-6-yl)benzyl)-2- | 411.2 |
| | hydroxybenzamide | |
| 355 | N-(3-(4-(cyclopropylamino)quinazolin-6-yl)benzyl)isonicotinamide | 396.2 |
| 356 | 2-cyano-N-(3-(4-(cyclopropylamino)quinazolin-6- | 358.2 |
| | yl)benzyl)acetamide | |
| 357 | N-(3-(4-(cyclopropylamino)quinazolin-6-yl)benzyl)-3- | 415.2 |
| | methylthiophene-2-carboxamide | |
| 358 | 3-cyano-N-(3-(4-(cyclopropylamino)quinazolin-6- | 420.2 |
| | yl)benzyl)benzamide | |
| 359 | N-(3-(4-(cyclopropylamino)quinazolin-6-yl)benzyl)-2-(pyridin-2- | 410.2 |
| | yl)acetamide | |
| 360 | N-(3-(4-(cyclopropylamino)quinazolin-6-yl)benzyl)pyrazine-2- | 397.2 |
| | carboxamide | |
| 361 | 4-chloro-N-(3-(4-(cyclopropylamino)quinazolin-6- | 429.1 |
| | yl)benzyl)benzamide | |
| 362 | N-(3-(4-(cyclopropylamino)quinazolin-6-yl)benzyl)-3,4- | 455.2 |
| | dimethoxybenzamide | |
| 363 | N-cyclopropyl-N-(3-(4-(3-methylbenzylamino)quinazolin-6- | 535.2 |
| | yl)benzyl)benzole sulfonamide | |
| 364 | N-cyclopropyl-2-fluoro-N-(3-(4-(3-methylbenzylamino)quinazolin- | 585.2 |
| | 6-yl)benzyl)-4-(trifluoromethyl)benzamide | |
| 365 | N-cyclopropyl-N-(3-(4-(3-methylbenzylamino)quinazolin-6- | 513.3 |
| | yl)benzyl)-2-phenylacetamide | |
| 366 | N-cyclopropyl-N-(5-(4-(cyclopropylamino)quinazolin-6-yl)-2- | 513.2 |
| | fluorobenzyl)-3,4-dimethoxybenzamide | |
| 367 | N-cyclopropyl-N-(5-(4-(cyclopropylamino)quinazolin-6-yl)-2- | 454.2 |
| | fluorobenzyl)nicotinamide | |
| 368 | N-cyclopropyl-N-(5-(4-(cyclopropylamino)quinazolin-6-yl)-2- | 489.2 |
| | fluorobenzyl)benzole sulfonamide | |
| 369 | N-cyclopropyl-1,3-dimethyl-N-(3-(4-(phenethylamino)quinazolin- | 517.3 |
| | 6-yl)benzyl)-1H-pyrazole-5-carboxamide | |
| 370 | N-cyclopropyl-N-(3-(4-(phenethylamino)quinazolin-6-yl)benzyl) | 500.2 |
| | picolinamide | |
| 371 | N-cyclopropyl-4-methoxy-N-(3-(4-(3- | 565.2 |
| | methylbenzylamino)quinazolin-6-yl)benzyl)benzole sulfonamide | |
| 372 | N-cyclopropyl-N-(3-(4-(3-methylbenzylamino)quinazolin-6- | 567.2 |
| | yl)benzyl)-3-(trifluoromethyl)benzamide | |
| 373 | N-cyclopropyl-N-(3-(4-(3-methylbenzylamino)quinazolin-6- | 527.3 |
| | yl)benzyl)-3-phenylpropanamide | |
| 374 | N-cyclopropyl-N-(5-(4-(cyclopropylamino)quinazolin-6-yl)-2- | 471.2 |
| | fluorobenzyl)-3-fluorobenzamide | |
| 375 | N-cyclopropyl-N-(5-(4-(cyclopropylamino)quinazolin-6-yl)-2- | 557.2 |
| | fluorobenzyl)-3-(trifluoromethyl)benzole sulfonamide | |
| 376 | N-cyclopropyl-N-(3-(4-(phenethylamino)quinazolin-6-yl)benzyl) | 499.2 |
| | benzamide | |
| 377 | N-cyclopropyl-N-(3-(4-(phenethylamino)quinazolin-6- | 500.2 |
| | yl)benzyl)isonicotinamide | |
| 378 | N-cyclopropyl-N-(3-(4-(3-methylbenzylamino)quinazolin-6- | 541.2 |
| | yl)benzyl)thiophene-2-sulfonamide | |
| 379 | N-cyclopropyl-N-(3-(4-(3-methylbenzylamino)quinazolin-6- | 567.2 |
| | yl)benzyl)-2-(trifluoromethyl)benzamide | |
| 380 | N-cyclopropyl-N-(3-(4-(3-methylbenzylamino)quinazolin-6- | 473.2 |
| | yl)benzyl)methane sulfonamide | |
| 381 | N-cyclopropyl-N-(5-(4-(cyclopropylamino)quinazolin-6-yl)-2- | 489.2 |
| | fluorobenzyl)-2,4-difluorobenzamide | |
| 382 | N-cyclopropyl-N-(5-(4-(cyclopropylamino)quinazolin-6-yl)-2- | 433.2 |
| | fluorobenzyl)pentanamide | |
| 383 | N-cyclopropyl-N-(5-(4-(cyclopropylamino)quinazolin-6-yl)-2- | 519.2 |
| | fluorobenzyl)-4-methoxybenzole sulfonamide | |
| 384 | N-cyclopropyl-3,4-dimethoxy-N-(3-(4- | 559.3 |
| | (phenethylamino)quinazolin-6-yl)benzyl)benzamide | |
| 385 | N-cyclopropyl-N-(3-(4-(phenethylamino)quinazolin-6- | 500.2 |
| | yl)benzyl)nicotinamide | |
| 386 | N-cyclopropyl-N-(3-(4-(3-methylbenzylamino)quinazolin-6- | 489.2 |
| | yl)benzyl)furane-2-carboxamide | |
| 387 | 3,5-dichloro-N-cyclopropyl-N-(3-(4-(3- | 567.2 |
| | methylbenzylamino)quinazolin-6-yl)benzyl)benzamide | |
| 388 | N-cyclopropyl-N-(3-(4-(3-methylbenzylamino)quinazolin-6- | 549.2 |
| | yl)benzyl)-1-phenylmethane sulfonamide | |
| 389 | N-cyclopropyl-N-(5-(4-(cyclopropylamino)quinazolin-6-yl)-2- | 543.2 |
| | fluorobenzyl)-2,3,4,5,6-pentafluorobenzamide | |
| 390 | N-cyclopropyl-N-(5-(4-(cyclopropylamino)quinazolin-6-yl)-2- | 421.2 |
| | fluorobenzyl)-2-methoxyacetamide | |
| 391 | 2,4-dichloro-N-cyclopropyl-N-(5-(4-(cyclopropylamino)- | 557.1 |
| | quinazolin-6-yl)-2-fluorobenzyl)benzole sulfonamide | |
| 392 | N-cyclopropyl-3-fluoro-N-(3-(4-(phenethylamino)quinazolin-6- | 517.2 |
| | yl)benzyl)benzamide | |
| 393 | N-cyclopropyl-N-(3-(4-(phenethylamino)quinazolin-6- | 437.2 |
| | yl)benzyl)acetamide | |
| 394 | N-cyclopropyl-N-(3-(4-(3-methylbenzylamino)quinazolin-6- | 505.2 |
| | yl)benzyl)thiophene-2-carboxamide | |
| 395 | N-cyclopropyl-N-(3-(4-(3-methylbenzylamino)quinazolin-6- | 500.2 |
| | yl)benzyl)picolinamide | |
| 396 | 2,4-dichloro-N-cyclopropyl-N-(3-(4-(3- | 603.1 |
| | methylbenzylamino)quinazolin-6-yl)benzyl)benzole sulfonamide | |
| 397 | N-cyclopropyl-N-(5-(4-(cyclopropylamino)quinazolin-6-yl)-2- | 539.2 |
| | fluorobenzyl)-2-fluoro-4-(trifluoromethyl)benzamide | |
| 398 | N-cyclopropyl-N-(5-(4-(cyclopropylamino)quinazolin-6-yl)-2- | 434.2 |
| | fluorobenzyl)-2-(dimethylamino)acetamide | |
| 399 | N-cyclopropyl-N-(5-(4-(cyclopropylamino)quinazolin-6-yl)-2- | 579.1 |
| | fluorobenzyl)-2,3,4,5,6-pentafluorobenzole sulfonamide | |
| 400 | N-cyclopropyl-2,4-difluoro-N-(3-(4-(phenethylamino)quinazolin-6- | 535.2 |
| | yl)benzyl)benzamide | |
| 401 | N-cyclopropyl-N-(3-(4-(phenethylamino)quinazolin-6- | 479.3 |
| | yl)benzyl)pivalamide | |
| 402 | N-cyclopropyl-1,3-dimethyl-N-(3-(4-(3-methylbenzylamino)- | 517.3 |
| | quinazolin-6-yl)benzyl)-1H-pyrazole-5-carboxamide | |
| 403 | N-cyclopropyl-N-(3-(4-(3-methylbenzylamino)quinazolin-6- | 500.2 |
| | yl)benzyl)isonicotinamide | |
| 404 | N-cyclopropyl-2,3,4,5,6-pentafluoro-N-(3-(4-(3- | 625.2 |
| | methylbenzylamino)quinazolin-6-yl)benzyl)benzole sulfonamide | |
| 405 | N-cyclopropyl-N-(5-(4-(cyclopropylamino)quinazolin-6-yl)-2- | 521.2 |
| | fluorobenzyl)-3-(trifluoromethyl)benzamide | |
| 406 | N-cyclopropyl-N-(5-(4-(cyclopropylamino)quinazolin-6-yl)-2- | 467.2 |
| | fluorobenzyl)-2-phenylacetamide | |
| 407 | N-cyclopropyl-N-(5-(4-(cyclopropylamino)quinazolin-6-yl)-2- | 495.1 |
| | fluorobenzyl)thiophene-2-sulfonamide | |
| 408 | N-cyclopropyl-2,3,4,5,6-pentafluoro-N-(3-(4-(phenethylamino) | 589.2 |
| | quinazolin-6-yl)benzyl)benzamide | |
| 409 | N-cyclopropyl-N-(3-(4-(phenethylamino)quinazolin-6- | 479.3 |
| | yl)benzyl)pentanamide | |
| 410 | N-cyclopropyl-N-(3-(4-(3-methylbenzylamino)quinazolin-6- | 499.2 |
| | yl)benzyl)benzamide | |
| 411 | N-cyclopropyl-N-(3-(4-(3-methylbenzylamino)quinazolin-6- | 500.2 |
| | yl)benzyl)nicotinamide | |
| 412 | N-cyclopropyl-N-(3-(4-(3-methylbenzylamino)quinazolin-6- | 586.2 |
| | yl)benzyl)quinoline-8-sulfonamide | |
| 413 | N-cyclopropyl-N-(5-(4-(cyclopropylamino)quinazolin-6-yl)-2- | 521.2 |
| | fluorobenzyl)-2-(trifluoromethyl)benzamide | |
| 414 | N-cyclopropyl-N-(5-(4-(cyclopropylamino)quinazolin-6-yl)-2- | 481.2 |
| | fluorobenzyl)-3-phenylpropanamide | |
| 415 | N-cyclopropyl-N-(5-(4-(cyclopropylamino)quinazolin-6-yl)-2- | 540.2 |
| | fluorobenzyl)quinoline-8-sulfonamide | |
| 416 | N-cyclopropyl-2-fluoro-N-(3-(4-(phenethylamino)quinazolin-6- | 585.2 |
| | yl)benzyl)-4-(trifluoromethyl)benzamide | |
| 417 | N-cyclopropyl-2-(dimethylamino)-N-(3-(4- | 480.3 |
| | (phenethylamino)quinazolin-6-yl)benzyl)acetamide | |
| 418 | N-cyclopropyl-3,4-dimethoxy-N-(3-(4-(3- | 559.3 |
| | methylbenzylamino)quinazolin-6-yl)benzyl)benzamide | |
| 419 | N-cyclopropyl-N-(3-(4-(3-methylbenzylamino)quinazolin-6- | 437.2 |
| | yl)benzyl)acetamide | |
| 420 | N-cyclopropyl-N-(5-(4-(cyclopropyl(methyl)amino)quinazolin-6- | 457.2 |
| | yl)-2-fluorobenzyl)furan-2-carboxamide | |
| 421 | 3,5-dichloro-N-cyclopropyl-N-(5-(4- | 521.1 |
| | (cyclopropylamino)quinazolin-6-yl)-2-fluorobenzyl)benzamide | |
| 422 | N-cyclopropyl-N-(3-(4-(phenethylamino)quinazolin-6- | 489.2 |
| | yl)benzyl)furan-2-carboxamide | |
| 423 | N-cyclopropyl-N-(3-(4-(phenethylamino)quinazolin-6-yl)benzyl)- | 567.2 |
| | 3-(trifluoromethyl)benzamide | |
| 424 | N-cyclopropyl-N-(3-(4-(phenethylamino)quinazolin-6-yl)benzyl)- | 513.3 |
| | 2-phenylacetamide | |
| 425 | N-cyclopropyl-3-fluoro-N-(3-(4-(3-methylbenzylamino)quinazolin- | 517.2 |
| | 6-yl)benzyl)benzamide | |
| 426 | N-cyclopropyl-N-(3-(4-(3-methylbenzylamino)quinazolin-6- | 479.3 |
| | yl)benzyl)pentanamide | |
| 427 | N-cyclopropyl-N-(5-(4-(cyclopropyl(methyl)amino)quinazolin-6- | 473.2 |
| | yl)-2-fluorobenzyl)thiophene-2-carboxamide | |
| 428 | N-cyclopropyl-N-(5-(4-(cyclopropylamino)quinazolin-6-yl)-2- | 454.2 |
| | fluorobenzyl)picolinamide | |
| 429 | N-cyclopropyl-N-(5-(4-(cyclopropylamino)quinazolin-6-yl)-2- | 455.2 |
| | fluorobenzyl)propane-2-sulfonamide | |
| 430 | N-cyclopropyl-N-(3-(4-(phenethylamino)quinazolin-6- | 505.2 |
| | yl)benzyl)thiophene-2-carboxamide | |
| 431 | N-cyclopropyl-N-(3-(4-(phenethylamino)quinazolin-6-yl)benzyl)- | 567.2 |
| | 2-(trifluoromethyl)benzamide | |
| 432 | N-cyclopropyl-N-(3-(4-(phenethylamino)quinazolin-6-yl)benzyl)- | 527.3 |
| | 3-phenylpropanamide | |
| 433 | N-cyclopropyl-2,4-difluoro-N-(3-(4-(3- | 535.2 |
| | methylbenzylamino)quinazolin-6-yl)benzyl)benzamide | |
| 434 | N-cyclopropyl-2-methoxy-N-(3-(4-(3- | 467.2 |
| | methylbenzylamino)quinazolin-6-yl)benzyl)acetamide | |
| 435 | N-cyclopropyl-N-(5-(4-(cyclopropylamino)quinazolin-6-yl)-2- | 471.2 |
| | fluorobenzyl)-1,3-dimethyl-1H-pyrazole-5-carboxamide | |
| 436 | N-cyclopropyl-N-(5-(4-(cyclopropylamino)quinazolin-6-yl)-2- | 454.2 |
| | fluorobenzyl)isonicotinamide | |
| 437 | N-cyclopropyl-N-(5-(4-(cyclopropylamino)quinazolin-6-yl)-2- | 503.2 |
| | fluorobenzyl)-1-phenylmethane sulfonamide | |
| 438 | N-cyclopropyl-N-(3-(4-(phenethylamino)quinazolin-6- | 490.2 |
| | yl)benzyl)isoxazole-5-carboxamide | |
| 439 | 3,5-dichloro-N-cyclopropyl-N-(3-(4-(phenethylamino)quinazolin- | 567.2 |
| | 6-yl)benzyl)benzamide | |
| 440 | N-cyclopropyl-N-(3-(4-(phenethylamino)quinazolin-6- | 473.2 |
| | yl)benzyl)methane sulfonamide | |
| 441 | N-cyclopropyl-N-(3-(4-(phenethylamino)quinazolin-6-yl)benzyl)- | 549.2 |
| | 1-phenyl methane sulfonamide | |
| 442 | N-cyclopropyl-2,3,4,5,6-pentafluoro-N-(3-(4-(3- | 589.2 |
| | methylbenzylamino)quinazolin-6-yl)benzyl)benzamide | |
| 443 | 2,3,4,5,6-pentafluoro-N-(3-(4-(methylamino)quinazolin-6- | 586.1 |
| | yl)benzyl)-N-(pyridin-2-ylmethyl)benzole sulfonamide | |
| 444 | N-(3-(4-(methylamino)quinazolin-6-yl)benzyl)-N-(pyridin-2- | 528.2 |
| | ylmethyl)-3-(trifluoromethyl)benzamide | |
| 445 | N-(3-(4-(methylamino)quinazolin-6-yl)benzyl)-3-phenyl-N- | 488.2 |
| | (pyridin-2-ylmethyl)propanamide | |
| 446 | N-(5-(4-(benzylamino)quinazolin-6-yl)-2-fluorobenzyl)-2,4- | 581.1 |
| | dichloro-N-methylbenzole sulfonamide | |
| 447 | N-cyclopropyl-N-(3-(4-(cyclopropylamino)-2-methylquinazolin-6- | 539.2 |
| | yl)benzyl)-2,3,4,5,6-pentafluorobenzamide | |
| 448 | 2,4-dichloro-N-cyclopropyl-N-(3-(4-(cyclopropylamino)-2- | 553.1 |
| | methylquinazolin-6-yl)benzyl)benzole sulfonamide | |
| 449 | N-cyclopropyl-N-(3-(4-(phenethylamino)quinazolin-6- | 535.2 |
| | yl)benzyl)benzole sulfonamide | |
| 450 | N-(3-(4-(methylamino)quinazolin-6-yl)benzyl)-N-(pyridin-2- | 547.2 |
| | ylmethyl)quinoline-8-sulfonamide | |
| 451 | 3,5-dichloro-N-(3-(4-(methylamino)quinazolin-6-yl)benzyl)-N- | 528.1 |
| | (pyridin-2-ylmethyl)benzamide | |
| 452 | N-cyclopropyl-N-(5-(4-(cyclopropylamino)quinazolin-6-yl)-2- | 459.3 |
| | fluorobenzyl)cyclohexane carboxamide | |
| 453 | N-(5-(4-(benzylamino)quinazolin-6-yl)-2-fluorobenzyl)-2,3,4,5,6- | 603.1 |
| | pentafluoro-N-methylbenzole sulfonamide | |
| 454 | N-cyclopropyl-N-(3-(4-(cyclopropylamino)-2-methylquinazolin-6- | 535.2 |
| | yl)benzyl)-2-fluoro-4-(trifluoromethyl)benzamide | |
| 455 | N-cyclopropyl-N-(3-(4-(cyclopropylamino)-2-methylquinazolin-6- | 575.2 |
| | yl)benzyl)-2,3,4,5,6-pentafluorobenzole sulfonamide | |
| 456 | N-cyclopropyl-N-(3-(4-(phenethylamino)quinazolin-6-yl)benzyl)- | 603.2 |
| | 3-(trifluoromethyl)benzole sulfonamide | |
| 457 | N-cyclopropyl-N-(5-(4-(cyclopropylamino)quinazolin-6-yl)-2- | 444.2 |
| | fluorobenzyl)isoxazole-5-carboxamide | |
| 458 | N-(3-(4-(methylamino)quinazolin-6-yl)benzyl)-N-(pyridin-2- | 450.2 |
| | ylmethyl)furan-2-carboxamide | |
| 459 | N-(3-(4-(methylamino)quinazolin-6-yl)benzyl)-N-(pyridin-2- | 461.2 |
| | ylmethyl)picolinamide | |
| 460 | N-cyclopropyl-N-(3-(4-(phenethylamino)quinazolin-6- | 505.3 |
| | yl)benzyl)cyclohexane carboxamide | |
| 461 | N-(5-(4-(benzylamino)quinazolin-6-yl)-2-fluorobenzyl)-N- | 519.1 |
| | methylthiophene-2-sulfonamide | |
| 462 | N-cyclopropyl-N-(3-(4-(cyclopropylamino)-2-methylquinazolin-6- | 517.2 |
| | yl)benzyl)-3-(trifluoromethyl)benzamide | |
| 463 | N-cyclopropyl-N-(3-(4-(cyclopropylamino)-2-methylquinazolin-6- | 491.2 |
| | yl)benzyl)thiophene-2-sulfonamide | |
| 464 | N-cyclopropyl-4-methoxy-N-(3-(4-(phenethylamino)quinazolin-6- | 565.2 |
| | yl)benzyl)benzole sulfonamide | |
| 465 | N-cyclopropyl-N-(3-(4-(3-methylbenzylamino)quinazolin-6- | 479.3 |
| | yl)benzyl)pivalamide | |
| 466 | N-(3-(4-(methylamino)quinazolin-6-yl)benzyl)-N-(pyridin-2- | 466.2 |
| | ylmethyl)thiophene-2-carboxamide | |
| 467 | N-(3-(4-(methylamino)quinazolin-6-yl)benzyl)-N-(pyridin-2- | 461.2 |
| | ylmethyl)isonicotinamide | |
| 468 | N-cyclopropyl-N-(3-(4-(3-methylbenzylamino)quinazolin-6- | 505.3 |
| | yl)benzyl)cyclohexane carboxamide | |
| 469 | N-(5-(4-(benzylamino)quinazolin-6-yl)-2-fluorobenzyl)-N- | 564.2 |
| | methylquinoline-8-sulfonamide | |
| 470 | N-cyclopropyl-N-(5-(4-(cyclopropylamino)-2-methylquinazolin-6- | 535.2 |
| | yl)-2-fluorobenzyl)-2-(trifluoromethyl)benzamide | |
| 471 | N-cyclopropyl-N-(3-(4-(cyclopropylamino)-2-methylquinazolin-6- | 536.2 |
| | yl)benzyl)quinoline-8-sulfonamide | |
| 472 | 2,4-dichloro-N-cyclopropyl-N-(3-(4-(phenethylamino)quinazolin- | 603.1 |
| | 6-yl)benzyl)benzole sulfonamide | |
| 473 | N-cyclopropyl-N-(3-(4-(3-methylbenzylamino)quinazolin-6- | 603.2 |
| | yl)benzyl)-3-(trifluoromethyl)benzole sulfonamide | |
| 474 | 1,3-dimethyl-N-(3-(4-(methylamino)quinazolin-6-yl)benzyl)-N- | 478.2 |
| | (pyridin-2-ylmethyl)-1H-pyrazole-5-carboxamide | |
| 475 | N-(3-(4-(methylamino)quinazolin-6-yl)benzyl)-N-(pyridin-2- | 461.2 |
| | ylmethyl)nicotinamide | |
| 476 | N-(5-(4-(benzylamino)quinazolin-6-yl)-2-fluorobenzyl)-N- | 451.2 |
| | methylmethane sulfonamide | |
| 477 | N-cyclopropyl-N-(3-(4-(cyclopropylamino)-2-methylquinazolin-6- | 440.2 |
| | yl)benzyl)isoxazole-5-carboxamide | |
| 478 | N-cyclopropyl-N-(3-(4-(cyclopropylamino)-2-methylquinazolin-6- | 423.2 |
| | yl)benzyl)methane sulfonamide | |
| 479 | 3,5-dichloro-N-cyclopropyl-N-(3-(4-(cyclopropylamino)-2- | 517.2 |
| | methylquinazolin-6-yl)benzyl)benzamide | |
| 480 | N-cyclopropyl-2,3,4,5,6-pentafluoro-N-(3-(4- | 625.2 |
| | (phenethylamino)quinazolin-6-yl)benzyl)benzole sulfonamide | |
| 481 | N-(3-(4-(methylamino)quinazolin-6-yl)benzyl)-N-(pyridin-2- | 434.2 |
| | ylmethyl)methane sulfonamide | |
| 482 | N-(3-(4-(methylamino)quinazolin-6-yl)benzyl)-N-(pyridin-2- | 460.2 |
| | ylmethyl)benzamide | |
| 483 | N-(3-(4-(methylamino)quinazolin-6-yl)benzyl)-N-(pyridin-2- | 398.2 |
| | ylmethyl)acetamide | |
| 484 | N-(5-(4-(benzylamino)quinazolin-6-yl)-2-fluorobenzyl)-N- | 479.2 |
| | methylpropane-2-sulfonamide | |
| 485 | N-cyclopropyl-N-(3-(4-(cyclopropylamino)-2-methylquinazolin-6- | 467.3 |
| | yl)benzyl)-1,3-dimethyl-1H-pyrazole-5-carboxamide | |
| 486 | N-cyclopropyl-N-(3-(4-(cyclopropylamino)-2-methylquinazolin-6- | 451.2 |
| | yl)benzyl)propane-2-sulfonamide | |
| 487 | N-cyclopropyl-N-(3-(4-(cyclopropylamino)-2-methylquinazolin-6- | 450.2 |
| | yl)benzyl)picolinamide | |
| 488 | N-cyclopropyl-N-(3-(4-(phenethylamino)quinazolin-6- | 541.2 |
| | yl)benzyl)thiophene-2-sulfonamide | |
| 489 | N-(3-(4-(methylamino)quinazolin-6-yl)benzyl)-1-phenyl-N- | 510.2 |
| | (pyridin-2-ylmethyl)methane sulfonamide | |
| 490 | 3,4-dimethoxy-N-(3-(4-(methylamino)quinazolin-6-yl)benzyl)-N- | 520.2 |
| | (pyridin-2-ylmethyl)benzamide | |
| 491 | N-(3-(4-(methylamino)quinazolin-6-yl)benzyl)-N-(pyridin-2- | 440.2 |
| | ylmethyl)pentanamide | |
| 492 | N-(5-(4-(benzylamino)quinazolin-6-yl)-2-fluorobenzyl)-N-methyl- | 527.2 |
| | 1-phenylmethane sulfonamide | |
| 493 | N-cyclopropyl-N-(3-(4-(cyclopropylamino)-2-methylquinazolin-6- | 449.2 |
| | yl)benzyl)benzamide | |
| 494 | N-cyclopropyl-N-(3-(4-(cyclopropylamino)-2-methylquinazolin-6- | 499.2 |
| | yl)benzyl)-1-phenylmethane sulfonamide | |
| 495 | N-cyclopropyl-N-(3-(4-(cyclopropylamino)-2-methylquinazolin-6- | 450.2 |
| | yl)benzyl)isonicotinamide | |
| 496 | N-cyclopropyl-N-(3-(4-(phenethylamino)quinazolin-6- | 586.2 |
| | yl)benzyl)quinoline-8-sulfonamide | |
| 497 | N-(3-(4-(methylamino)quinazolin-6-yl)benzyl)-N-(pyridin-2- | 496.2 |
| | ylmethyl)benzole sulfonamide | |
| 498 | 3-fluoro-N-(3-(4-(methylamino)quinazolin-6-yl)benzyl)-N- | 478.2 |
| | (pyridin-2-ylmethyl)benzamide | |
| 499 | 2-methoxy-N-(3-(4-(methylamino)quinazolin-6-yl)benzyl)-N- | 428.2 |
| | (pyridin-2-ylmethyl)acetamide | |
| 500 | N-(5-(4-(benzylamino)quinazolin-6-yl)-2-fluorobenzyl)-N- | 513.2 |
| | methylbenzole sulfonamide | |
| 501 | N-cyclopropyl-N-(3-(4-(cyclopropylamino)-2-methylquinazolin-6- | 509.3 |
| | yl)benzyl)-3,4-dimethoxybenzamide | |
| 502 | N-cyclopropyl-N-(3-(4-(cyclopropylamino)-2-methylquinazolin-6- | 485.2 |
| | yl)benzyl)benzole sulfonamide | |
| 503 | N-cyclopropyl-N-(3-(4-(cyclopropylamino)-2-methylquinazolin-6- | 450.2 |
| | yl)benzyl)nicotinamide | |
| 504 | N-cyclopropyl-N-(3-(4-(3-methylbenzylamino)quinazolin-6- | 501.2 |
| | yl)benzyl)propane-2-sulfonamide | |
| 505 | N-(3-(4-(methylamino)quinazolin-6-yl)benzyl)-N-(pyridin-2- | 564.2 |
| | ylmethyl)-3-(trifluoromethyl)benzole sulfonamide | |
| 506 | 2,4-difluoro-N-(3-(4-(methylamino)quinazolin-6-yl)benzyl)-N- | 496.2 |
| | (pyridin-2-ylmethyl)benzamide | |
| 507 | N-(3-(4-(methylamino)quinazolin-6-yl)benzyl)-N-(pyridin-2- | 466.3 |
| | ylmethyl)cyclohexane carboxamide | |
| 508 | N-(5-(4-(benzylamino)quinazolin-6-yl)-2-fluorobenzyl)-N-methyl- | 581.2 |
| | 3-(trifluoromethyl)benzole sulfonamide | |
| 509 | N-cyclopropyl-N-(3-(4-(cyclopropylamino)-2-methylquinazolin-6- | 467.2 |
| | yl)benzyl)-3-fluorobenzamide | |
| 510 | N-cyclopropyl-N-(3-(4-(cyclopropylamino)-2-methylquinazolin-6- | 553.2 |
| | yl)benzyl)-3-(trifluoromethyl)benzole sulfonamide | |
| 511 | N-cyclopropyl-N-(3-(4-(cyclopropylamino)-2-methylquinazolin-6- | 387.2 |
| | yl)benzyl)acetamide | |
| 512 | N-cyclopropyl-N-(3-(4-(3-methylbenzylamino)quinazolin-6- | 490.2 |
| | yl)benzyl)isoxazole-5-carboxamide | |
| 513 | 4-methoxy-N-(3-(4-(methylamino)quinazolin-6-yl)benzyl)-N- | 526.2 |
| | (pyridin-2-ylmethyl)benzole sulfonamide | |
| 514 | 2-fluoro-N-(3-(4-(methylamino)quinazolin-6-yl)benzyl)-N- | 546.2 |
| | (pyridin-2-ylmethyl)-4-(trifluoromethyl)benzamide | |
| 515 | N-(3-(4-(methylamino)quinazolin-6-yl)benzyl)-2-phenyl-N- | 474.2 |
| | (pyridin-2-ylmethyl)acetamide | |
| 516 | N-(5-(4-(benzylamino)quinazolin-6-yl)-2-fluorobenzyl)-4- | 543.2 |
| | methoxy-N-methylbenzole sulfonamide | |
| 517 | N-cyclopropyl-N-(3-(4-(cyclopropylamino)-2-methylquinazolin-6- | 485.2 |
| | yl)benzyl)-2,4-difluorobenzamide | |
| 518 | N-cyclopropyl-N-(3-(4-(cyclopropylamino)-2-methylquinazolin-6- | 515.2 |
| | yl)benzyl)-4-methoxybenzole sulfonamide | |
| 519 | N-cyclopropyl-N-(3-(4-(cyclopropylamino)-2-methylquinazolin-6- | 429.3 |
| | yl)benzyl)pentanamide | |
| 520 | N-cyclopropyl-N-(3-(4-(cyclopropylamino)-2-methylquinazolin-6- | 417.2 |
| | yl)benzyl)-2-methoxyacetamide | |
| 521 | N-(2-fluoro-5-(4-(methylamino)quinazolin-6-yl)benzyl)-N- | 391.2 |
| | methylfuran-2-carboxamide | |
| 522 | N-(2-fluoro-5-(4-(methylamino)quinazolin-6-yl)benzyl)-N-methyl- | 469.2 |
| | 3-(trifluoromethyl)benzamide | |
| 523 | N-(2-fluoro-5-(4-(methylamino)quinazolin-6-yl)benzyl)-N-methyl- | 505.1 |
| | 3-(trifluoromethyl)benzole sulfonamide | |
| 524 | N-cyclopropyl-N-(5-(4-(cyclopropylamino)-2-methylquinazolin-6- | 503.2 |
| | yl)-2-fluorobenzyl)-2,4-difluorobenzamide | |
| 525 | N-(5-(4-(benzylamino)quinazolin-6-yl)-2-fluorobenyl)-N-methyl- | 545.2 |
| | 2-(trifluoromethyl)benzamide | |
| 526 | N-cyclopropyl-N-(5-(4-(cyclopropylamino)-2-methylquinazolin-6- | 457.2 |
| | yl)-2-fluorobenzyl)furan-2-carboxamide | |
| 527 | N-cyclopropyl-N-(5-(4-(cyclopropylamino)-2-methylquinazolin-6- | 441.2 |
| | yl)-2-fluorobenzyl)methane sulfonamide | |
| 528 | N-cyclopropyl-N-(3-(4-(cyclopropylamino)-2-methylquinazolin-6- | 430.3 |
| | yl)benzyl)-2-(dimethylamino)acetamide | |
| 529 | N-(2-fluoro-5-(4-(methylamino)quinazolin-6-yl)benzyl)-N- | 407.1 |
| | methylthiophene-2-carboxamide | |
| 530 | N-(2-fluoro-5-(4-(methylamino)quinazolin-6-yl)benzyl)-N-methyl- | 469.2 |
| | 2-(trifluoromethyl)benzamide | |
| 531 | 2,4-dichloro-N-(2-fluoro-5-(4-(methylamino)quinazolin-6- | 505.1 |
| | yl)benzyl)-N-methylbenzole sulfonamide | |
| 532 | N-cyclopropyl-N-(5-(4-(cyclopropylamino)-2-methylquinazolin-6- | 553.2 |
| | yl)-2-fluorobenzyl)-2-fluoro-4-(trifluoromethyl)benzamide | |
| 533 | N-(5-(4-(benzylamino)quinazolin-6-yl)-2-fluorobenzyl)-3,5- | 545.1 |
| | dichloro-N-methylbenzamide | |
| 534 | N-cyclopropyl-N-(5-(4-(cyclopropylamino)-2-methylquinazolin-6- | 473.2 |
| | yl)-2-fluorobenzyl)thiophene-2-carboxamide | |
| 535 | N-cyclopropyl-N-(5-(4-(cyclopropylamino)-2-methylquinazolin-6- | 469.2 |
| | yl)-2-fluorobenzyl)propane-2-sulfonamide | |
| 536 | N-cyclopropyl-N-(3-(4-(cyclopropylamino)-2-methylquinazolin-6- | 455.3 |
| | yl)benzyl)cyclohexane carboxamide | |
| 537 | N-(2-fluoro-5-(4-(methylamino)quinazolin-6-yl)benzyl)-N- | 392.2 |
| | methylisoxazole-5-carboxamide | |
| 538 | N-(2-fluoro-5-(4-(methylamino)quinazolin-6-yl)benzyl)-N- | 339.2 |
| | methylacetamide | |
| 539 | 2,3,4,5,6-pentafluoro-N-(2-fluoro-5-(4-(methylamino)quinazolin-6- | 527.1 |
| | yl)benzyl)-N-methylbenzole sulfonamide | |
| 540 | N-cyclopropyl-N-(5-(4-(cyclopropylamino)-2-methylquinazolin-6- | 447.3 |
| | yl)-2-fluorobenzyl)pentanamide | |
| 541 | N-(5-(4-(benzylamino)quinazolin-6-yl)-2-fluorobenzyl)-N- | 457.2 |
| | methylpivalamide | |
| 542 | N-cyclopropyl-N-(5-(4-(cyclopropylamino)-2-methylquinazolin-6- | 485.2 |
| | yl)-2-fluorobenzyl)-1,3-dimethyl-1H-pyrazole-5-carboxamide | |
| 543 | N-cyclopropyl-N-(5-(4-(cyclopropylamino)-2-methylquinazolin-6- | 517.2 |
| | yl)-2-fluorobenzyl)-1-phenylmethane sulfonamide | |
| 544 | N-cyclopropyl-N-(3-(4-(cyclopropylamino)-2-methylquinazolin-6- | 463.2 |
| | yl)benzyl)-2-phenylacetamide | |
| 545 | N-(2-fluoro-5-(4-(methylamino)quinazolin-6-yl)benzyl)-N,1,3- | 419.2 |
| | trimethyl-1H-pyrazole-5-carboxamide | |
| 546 | N-(2-fluoro-5-(4-(methylamino)quinazolin-6-yl)benzyl)-N- | 407.2 |
| | methylcyclohexane carboxamide | |
| 547 | N-(2-fluoro-5-(4-(methylamino)quinazolin-6-yl)benzyl)-N- | 443.1 |
| | methylthiophene-2-sulfonamide | |
| 548 | N-(5-(4-(benzylamino)quinazolin-6-yl)-2-fluorobenzyl)-N,1,3- | 495.2 |
| | trimethyl-1H-pyrazole-5-carboxamide | |
| 549 | N-(5-(4-(benzylamino)quinazolin-6-yl)-2-fluorobenzyl)-N- | 457.2 |
| | methylpentanamide | |
| 550 | N-cyclopropyl-N-(5-(4-(cyclopropylamino)-2-methylquinazolin-6- | 467.2 |
| | yl)-2-fluorobenzyl)benzamide | |
| 551 | N-cyclopropyl-N-(5-(4-(cyclopropylamino)-2-methylquinazolin-6- | 503.2 |
| | yl)-2-fluorobenzyl)benzole sulfonamide | |
| 552 | N-cyclopropyl-N-(3-(4-(cyclopropylamino)-2-methylquinazolin-6- | 477.3 |
| | yl)benzyl)-3-phenylpropanamide | |
| 553 | N-(2-fluoro-5-(4-(methylamino)quinazolin-6-yl)benzyl)-N- | 401.2 |
| | methylbenzamide | |
| 554 | N-(2-fluoro-5-(4-(methylamino)quinazolin-6-yl)benzyl)-2- | 369.2 |
| | methoxy-N-methylacetamide | |
| 555 | N-(2-fluoro-5-(4-(methylamino)quinazolin-6-yl)benzyl)-N- | 488.2 |
| | methylquinoline-8-sulfonamide | |
| 556 | N-(5-(4-(benzylamino)quinazolin-6-yl)-2-fluorobenzyl)-N- | 477.2 |
| | methylbenzamide | |
| 557 | N-(5-(4-(benzylamino)quinazolin-6-yl)-2-fluorobenzyl)-2- | 445.2 |
| | methoxy-N-methylacetamide | |
| 558 | N-cyclopropyl-N-(5-(4-(cyclopropylamino)-2-methylquinazolin-6- | 527.2 |
| | yl)-2-fluorobenzyl)-3,4-dimethoxybenzamide | |
| 559 | N-cyclopropyl-N-(5-(4-(cyclopropylamino)-2-methylquinazolin-6- | 571.2 |
| | yl)-2-fluorobenzyl)-3-(trifluoromethyl)benzole sulfonamide | |
| 560 | N-(2-fluoro-5-(4-(methylamino)quinazolin-6-yl)benzyl)-4- | 467.2 |
| | methoxy-N-methylbenzole sulfonamide | |
| 561 | N-(2-fluoro-5-(4-(methylamino)quinazolin-6-yl)benzyl)-3,4- | 461.2 |
| | dimethoxy-N-methylbenzamide | |
| 562 | N-(2-fluoro-5-(4-(methylamino)quinazolin-6-yl)benzyl)-N-methyl- | 429.2 |
| | 3-phenylpropanamide | |
| 563 | N-(5-(4-(benzylamino)quinazolin-6-yl)-2-fluorobenzyl)-N- | 483.2 |
| | methylthiophene-2-carboxamide | |
| 564 | N-(5-(4-(benzylamino)quinazolin-6-yl)-2-fluorobenzyl)-3,4- | 537.2 |
| | dimethoxy-N-methylbenzamide | |
| 565 | N-(5-(4-(benzylamino)quinazolin-6-yl)-2-fluorobenzyl)-2- | 458.2 |
| | (dimethylamino)-N-methylacetamide | |
| 566 | N-cyclopropyl-N-(5-(4-(cyclopropylamino)-2-methylquinazolin-6- | 485.2 |
| | yl)-2-fluorobenzyl)-3-fluorobenzamide | |
| 567 | N-cyclopropyl-N-(5-(4-(cyclopropylamino)-2-methylquinazolin-6- | 533.2 |
| | yl)-2-fluorobenzyl)-4-methoxybenzole sulfonamide | |
| 568 | N-(2-fluoro-5-(4-(methylamino)quinazolin-6-yl)benzyl)-N-methyl | 402.2 |
| | isonicotinamide | |
| 569 | 3-fluoro-N-(2-fluoro-5-(4-(methylamino)quinazolin-6-yl)benzyl)- | 419.2 |
| | N-methylbenzamide | |
| 570 | N-(2-fluoro-5-(4-(methylamino)quinazolin-6-yl)benzyl)-N- | 375.1 |
| | methylmethane sulfonamide | |
| 571 | N-(5-(4-(benzylamino)quinazolin-6-yl)-2-fluorobenzyl)-3-fluoro- | 495.2 |
| | N-methylbenzamide | |
| 572 | N-(5-(4-(benzylamino)quinazolin-6-yl)-2-fluorobenzyl)-2,4- | 513.2 |
| | difluoro-N-methylbenzamide | |
| 573 | N-(2-fluoro-5-(4-(methylamino)quinazolin-6-yl)benzyl)-N- | 402.2 |
| | methylnicotinamide | |
| 574 | N-cyclopropyl-N-(5-(4-(cyclopropylamino)-2-methylquinazolin-6- | 557.2 |
| | yl)-2-fluorobenzyl)-2,3,4,5,6-pentafluorobenzamide | |
| 575 | 2,4-dichloro-N-cyclopropyl-N-(5-(4-(cyclopropylamino)-2- | 571.1 |
| | methylquinazolin-6-yl)-2-fluorobenzyl)benzole sulfonamide | |
| 576 | N-(2-fluoro-5-(4-(methylamino)quinazolin-6-yl)benzyl)-N- | 381.2 |
| | methylpentanamide | |
| 577 | 2,4-difluoro-N-(2-fluoro-5-(4-(methylamino)quinazolin-6- | 437.2 |
| | yl)benzyl)-N-methylbenzamide | |
| 578 | N-(2-fluoro-5-(4-(methylamino)quinazolin-6-yl)benzyl)-N- | 403.2 |
| | methylpropane-2-sulfonamide | |
| 579 | N-(5-(4-(benzylamino)quinazolin-6-yl)-2-fluorobenzyl)-N- | 478.2 |
| | methylisonicotinamide | |
| 580 | N-(5-(4-(benzylamino)quinazolin-6-yl)-2-fluorobenzyl)-2,3,4,5,6- | 567.2 |
| | pentafluoro-N-methylbenzamide | |
| 581 | N-(5-(4-(benzylamino)quinazolin-6-yl)-2-fluorobenzyl)-N- | 483.3 |
| | methylcyclohexane carboxamide | |
| 582 | N-cyclopropyl-N-(5-(4-(cyclopropylamino)-2-methylquinazolin-6- | 535.2 |
| | yl)-2-fluorobenzyl)-3-(trifluoromethyl)benzamide | |
| 583 | N-cyclopropyl-N-(5-(4-(cyclopropylamino)-2-methylquinazolin-6- | 593.1 |
| | yl)-2-fluorobenzyl)-2,3,4,5,6-pentafluorobenzole sulfonamide | |
| 584 | N-(2-fluoro-5-(4-(methylamino)quinazolin-6-yl)benzyl)-N-methyl- | 415.2 |
| | 2-phenylacetamide | |
| 585 | 2,3,4,5,6-pentafluoro-N-(2-fluoro-5-(4-(methylamino)quinazolin-6- | 491.1 |
| | yl)benzyl)-N-methylbenzamide | |
| 586 | N-(2-fluoro-5-(4-(methylamino)quinazolin-6-yl)benzyl)-N-methyl- | 451.2 |
| | 1-phenylmethane sulfonamide | |
| 587 | N-(5-(4-(benzylamino)quinazolin-6-yl)-2-fluorobenzyl)-N- | 478.2 |
| | methylnicotinamide | |
| 588 | N-(5-(4-(benzylamino)quinazolin-6-yl)-2-fluorobenzyl)-2-fluoro- | 563.2 |
| | N-methyl-4-(trifluoromethyl)benzamide | |
| 589 | N-(5-(4-(benzylamino)quinazolin-6-yl)-2-fluorobenzyl)-N-methyl- | 491.2 |
| | 2-phenylacetamide | |
| 590 | N-(2-fluoro-5-(4-(methylamino)quinazolin-6-yl)benzyl)-N- | 381.2 |
| | methylpivalamide | |
| 591 | N-cyclopropyl-N-(5-(4-(cyclopropylamino)-2-methylquinazolin-6- | 509.1 |
| | yl)-2-fluorobenzyl)thiophene-2-sulfonamide | |
| 592 | N-(2-fluoro-5-(4-(methylamino)quinazolin-6-yl)benzyl)-N- | 402.2 |
| | methylpicolinamide | |
| 593 | 2-fluoro-N-(2-fluoro-5-(4-(methylamino)quinazolin-6-yl)benzyl)- | 487.2 |
| | N-methyl-4-(trifluoromethyl)benzamide | |
| 594 | N-(2-fluoro-5-(4-(methylamino)quinazolin-6-yl)benzyl)-N- | 437.1 |
| | methylbenzole sulfonamide | |
| 595 | N-(5-(4-(benzylamino)quinazolin-6-yl)-2-fluorobenzyl)-N- | 415.2 |
| | methylacetamide | |
| 596 | N-(5-(4-(benzylamino)quinazolin-6-yl)-2-fluorobenzyl)-N-methyl- | 545.2 |
| | 3-(trifluoromethyl)benzamide | |
| 597 | N-(5-(4-(benzylamino)quinazolin-6-yl)-2-fluorobenzyl)-N-methyl- | 505.2 |
| | 3-phenylpropanamide | |
| 598 | N-(5-(4-(benzylamino)quinazolin-6-yl)-2-fluorobenzyl)-N- | 467.2 |
| | methylfuran-2-carboxamide | |
| 599 | N-cyclopropyl-N-(5-(4-(cyclopropylamino)-2-methylquinazolin-6- | 554.2 |
| | yl)-2-fluorobenzyl)quinoline-8-sulfonamide | |
| 600 | 3,5-dichloro-N-cyclopropyl-N-(5-(4-(cyclopropylamino)-2- | 535.1 |
| | methylquinazolin-6-yl)-2-fluorobenzyl)benzamide | |
| 601 | N-cyclopropyl-N-(5-(4-(cyclopropylamino)-2-methylquinazolin-6- | 495.3 |
| | yl)-2-fluorobenzyl)-3-phenylpropanamide | |
| 602 | N-cyclopropyl-N-(3-(4-(2,4-dimethoxybenzylamino)quinazolin-6- | 581.2 |
| | yl)benzyl)benzole sulfonamide | |
| 603 | N-cyclopropyl-N-(3-(4-(2,4-dimethoxybenzylamino)quinazolin-6- | 546.3 |
| | yl)benzyl)isonicotinamide | |
| 604 | N-cyclopropyl-N-(5-(4-(cyclopropylamino)-2-methylquinazolin-6- | 468.2 |
| | yl)-2-fluorobenzyl)picolinamide | |
| 605 | N-cyclopropyl-N-(5-(4-(cyclopropylamino)quinazolin-6-yl)-2- | 474.2 |
| | fluorobenzyl)-2-methylthiazole-4-carboxamide | |
| 606 | N-cyclopropyl-N-(3-(4-(2,4-dimethoxybenzylamino)quinazolin-6- | 649.2 |
| | yl)benzyl)-3-(trifluoromethyl)benzole sulfonamide | |
| 607 | N-cyclopropyl-N-(3-(4-(2,4-dimethoxybenzylamino)quinazolin-6- | 545.3 |
| | yl)benzyl)benzamide | |
| 608 | N-cyclopropyl-N-(5-(4-(cyclopropylamino)-2-methylquinazolin-6- | 468.2 |
| | yl)-2-fluorobenzyl)isonicotinamide | |
| 609 | N-cyclopropyl-2-methyl-N-(3-(4-(phenethylamino)quinazolin-6- | 520.2 |
| | yl)benzyl)thiazole-4-carboxamide | |
| 610 | N-cyclopropyl-N-(3-(4-(2,4-dimethoxybenzylamino)quinazolin-6- | 611.2 |
| | yl)benzyl)-4-methoxybenzole sulfonamide | |
| 611 | N-cyclopropyl-N-(3-(4-(2,4-dimethoxybenzylamino)quinazolin-6- | 563.2 |
| | yl)benzyl)-3-fluorobenzamide | |
| 612 | N-cyclopropyl-N-(5-(4-(cyclopropylamino)-2-methylquinazolin-6- | 468.2 |
| | yl)-2-fluorobenzyl)nicotinamide | |
| 613 | N-cyclopropyl-N-(3-(4-(2,4-dimethoxybenzylamino)quinazolin-6- | 566.2 |
| | yl)benzyl)-2-methylthiazole-4-carboxamide | |
| 614 | 2,4-dichloro-N-cyclopropyl-N-(3-(4-(2,4-dimethoxybenzylamino) | 649.1 |
| | quinazolin-6-yl)benzyl)benzole sulfonamide | |
| 615 | N-cyclopropyl-N-(3-(4-(2,4-dimethoxybenzylamino)quinazolin-6- | 581.2 |
| | yl)benzyl)-2,4-difluorobenzamide | |
| 616 | N-cyclopropyl-N-(5-(4-(cyclopropylamino)-2-methylquinazolin-6- | 405.2 |
| | yl)-2-fluorobenzyl)acetamide | |
| 617 | N-(2-fluoro-5-(4-(methylamino)quinazolin-6-yl)benzyl)-N,2- | 422.1 |
| | dimethylthiazole-4-carboxamide | |
| 618 | N-cyclopropyl-2-methyl-N-(3-(4-(3-methylbenzylamino)- | 520.2 |
| | quinazolin-6-yl)benzyl)thiazole-4-carboxamide | |
| 619 | N-cyclopropyl-N-(3-(4-(cyclopropylamino)-8-fluoroquinazolin-6- | 489.2 |
| | yl)benzyl)-2,4-difluorobenzamide | |
| 620 | N-cyclopropyl-N-(3-(4-(2,4-dimethoxybenzylamino)quinazolin-6- | 635.2 |
| | yl)benzyl)-2,3,4,5,6-pentafluorobenzamide | |
| 621 | N-cyclopropyl-N-(5-(4-(cyclopropylamino)-2-methylquinazolin-6- | 447.3 |
| | yl)-2-fluorobenzyl)pivalamide | |
| 622 | N-(5-(4-(benzylamino)quinazolin-6-yl)-2-fluorobenzyl)-N,2- | 498.2 |
| | dimethylthiazole-4-carboxamide | |
| 623 | 2,4-dichloro-N-(3-(4-(methylamino)quinazolin-6-yl)benzyl)-N- | 564.1 |
| | (pyridin-2-ylmethyl)benzole sulfonamide | |
| 624 | N-cyclopropyl-N-(5-(4-(cyclopropylamino)-2-methylquinazolin-6- | 488.2 |
| | yl)-2-fluorobenzyl)-2-methylthiazole-4-carboxamide | |
| 625 | N-cyclopropyl-N-(3-(4-(2,4-dimethoxybenzylamino)quinazolin-6- | 587.2 |
| | yl)benzyl)thiophene-2-sulfonamide | |
| 626 | N-cyclopropyl-N-(3-(4-(2,4-dimethoxybenzylamino)quinazolin-6- | 631.2 |
| | yl)benzyl)-2-fluoro-4-(trifluoromethyl)benzamide | |
| 627 | N-cyclopropyl-N-(5-(4-(cyclopropylamino)-2-methylquinazolin-6- | 435.2 |
| | yl)-2-fluorobenzyl)-2-methoxyacetamide | |
| 628 | N-(3-(4-(methylamino)quinazolin-6-yl)benzyl)-N-(pyridin-2- | 440.2 |
| | ylmethyl)pivalamide | |
| 629 | N-(3-(4-(methylamino)quinazolin-6-yl)benzyl)-N-(pyridin-2- | 502.1 |
| | ylmethyl)thiophene-2-sulfonamide | |
| 630 | 2-methyl-N-(3-(4-(methylamino)quinazolin-6-yl)benzyl)-N- | 481.2 |
| | (pyridin-2-ylmethyl)thiazole-4-carboxamide | |
| 631 | N-cyclopropyl-N-(3-(4-(2,4-dimethoxybenzylamino)quinazolin-6- | 632.2 |
| | yl)benzyl)quinoline-8-sulfonamide | |
| 632 | N-cyclopropyl-N-(3-(4-(2,4-dimethoxybenzylamino)quinazolin-6- | 613.2 |
| | yl)benzyl)-3-(trifluoromethyl)benzamide | |
| 633 | N-cyclopropyl-N-(5-(4-(cyclopropylamino)-2-methylquinazolin-6- | 448.3 |
| | yl)-2-fluorobenzyl)-2-(dimethylamino)acetamide | |
| 634 | N-cyclopropyl-N-(3-(4-(cyclopropylamino)-2-methylquinazolin-6- | 470.2 |
| | yl)benzyl)-2-methylthiazole-4-carboxamide | |
| 635 | N-(3-(4-(methylamino)quinazolin-6-yl)benzyl)-N-(pyridin-2- | 462.2 |
| | ylmethyl)propane-2-sulfonamide | |
| 636 | N-(5-(4-(benzylamino)quinazolin-6-yl)-2-fluorobenzyl)-N- | 478.2 |
| | methylpicolinamide | |
| 637 | N-cyclopropyl-N-(3-(4-(2,4-dimethoxybenzylamino)quinazolin-6- | 535.2 |
| | yl)benzyl)furan-2-carboxamide | |
| 638 | N-cyclopropyl-N-(3-(4-(2,4-dimethoxybenzylamino)quinazolin-6- | 613.2 |
| | yl)benzyl)-2-(trifluoromethyl)benzamide | |
| 639 | N-cyclopropyl-N-(5-(4-(cyclopropylamino)-2-methylquinazolin-6- | 473.3 |
| | yl)-2-fluorobenzyl)cyclohexane carboxamide | |
| 640 | N-cyclopropyl-N-(3-(4-(cyclopropylamino)-2-methylquinazolin-6- | 429.3 |
| | yl)benzyl)pivalamide | |
| 641 | N-cyclopropyl-N-(3-(4-(2,4-dimethoxybenzylamino)quinazolin-6- | 519.2 |
| | yl)benzyl)methane sulfonamide | |
| 642 | N-cyclopropyl-N-(3-(4-(2,4-dimethoxybenzylamino)quinazolin-6- | 551.2 |
| | yl)benzyl)thiophene-2-carboxamide | |
| 643 | 3,5-dichloro-N-cyclopropyl-N-(3-(4-(2,4- | 613.2 |
| | dimethoxybenzylamino)quinazolin-6-yl)benzyl)benzamide | |
| 644 | N-cyclopropyl-N-(5-(4-(cyclopropylamino)-2-methylquinazolin-6- | 481.2 |
| | yl)-2-fluorobenzyl)-2-phenylacetamide | |
| 645 | N-cyclopropyl-N-(5-(4-(cyclopropylamino)quinazolin-6-yl)-2- | 433.2 |
| | fluorobenzyl)pivalamide | |
| 646 | N-cyclopropyl-N-(3-(4-(2,4-dimethoxybenzylamino)quinazolin-6- | 595.2 |
| | yl)benzyl)-1-phenylmethane sulfonamide | |
| 647 | N-cyclopropyl-N-(3-(4-(2,4-dimethoxybenzylamino)quinazolin-6- | 536.2 |
| | yl)benzyl)isoxazole-5-carboxamide | |
| 648 | N-cyclopropyl-N-(3-(4-(2,4-dimethoxybenzylamino)quinazolin-6- | 546.3 |
| | yl)benzyl)picolinamide | |
| 649 | N-cyclopropyl-N-(3-(4-(2,4-dimethoxybenzylamino)quinazolin-6- | 546.3 |
| | yl)benzyl)nicotinamide | |
| 650 | N-cyclopropyl-N-(3-(4-(cyclopropylamino)-2-methylquinazolin-6- | 439.2 |
| | yl)benzyl)furan-2-carboxamide | |
| 651 | N-cyclopropyl-N-(3-(4-(cyclopropylamino)-7-methylquinazolin-6- | 553.2 |
| | yl)benzyl)-3-(trifluoromethyl)benzole sulfonamide | |
| 652 | N-cyclopropyl-N-(3-(4-(cyclopropylamino)-7-methylquinazolin-6- | 467.2 |
| | yl)benzyl)-3-fluorobenzamide | |
| 653 | N-cyclopropyl-N-(3-(4-(cyclopropylamino)-7-methylquinazolin-6- | 455.3 |
| | yl)benzyl)cyclohexane carboxamide | |
| 654 | N-cyclopropyl-N-(3-(4-(cyclopropylamino)-8-fluoroquinazolin-6- | 433.2 |
| | yl)benzyl)pentanamide | |
| 655 | N-(2-fluorophenyl)-N-(3-(4-(methylamino)quinazolin-6- | 505.1 |
| | yl)benzyl)thiophene-2-sulfonamide | |
| 656 | N-cyclopropyl-N-(3-(4-(2,4-dimethoxybenzylamino)quinazolin-6- | 605.3 |
| | yl)benzyl)-3,4-dimethoxybenzamide | |
| 657 | N-cyclopropyl-N-(3-(4-(2,4-dimethoxybenzylamino)quinazolin-6- | 483.2 |
| | yl)benzyl)acetamide | |
| 658 | N-cyclopropyl-N-(3-(4-(cyclopropylamino)-2-methylquinazolin-6- | 455.2 |
| | yl)benzyl)thiophene-2-carboxamide | |
| 659 | N-cyclopropyl-N-(3-(4-(cyclopropylamino)-7-methylquinazolin-6- | 515.2 |
| | yl)benzyl)-4-methoxybenzole sulfonamide | |
| 660 | N-cyclopropyl-N-(3-(4-(cyclopropylamino)-7-methylquinazolin-6- | 485.2 |
| | yl)benzyl)-2,4-difluorobenzamide | |
| 661 | N-cyclopropyl-N-(3-(4-(cyclopropylamino)-7-methylquinazolin-6- | 463.2 |
| | yl)benzyl)-2-phenylacetamide | |
| 662 | N-cyclopropyl-N-(3-(4-(cyclopropylamino)-8-fluoroquinazolin-6- | 459.3 |
| | yl)benzyl)cyclohexane carboxamide | |
| 663 | N-(2-fluorophenyl)-N-(3-(4-(methylamino)quinazolin-6- | 550.2 |
| | yl)benzyl)quinoline-8-sulfonamide | |
| 664 | N-(5-(4-(benzylamino)quinazolin-6-yl)-2-fluorobenzyl)-N- | 468.2 |
| | methylisoxazole-5-carboxamide | |
| 665 | N-cyclopropyl-N-(3-(4-(2,4-dimethoxybenzylamino)quinazolin-6- | 525.3 |
| | yl)benzyl)pivalamide | |
| 666 | N-cyclopropyl-N-(3-(4-(cyclopropylamino)-2-methylquinazolin-6- | 517.2 |
| | yl)benzyl)-2-(trifluoromethyl)benzamide | |
| 667 | 2,4-dichloro-N-cyclopropyl-N-(3-(4-(cyclopropylamino)-7- | 553.1 |
| | methylquinazolin-6-yl)benzyl)benzole sulfonamide | |
| 668 | N-cyclopropyl-N-(3-(4-(cyclopropylamino)-7-methylquinazolin-6- | 535.2 |
| | yl)benzyl)-2-fluoro-4-(trifluoromethyl)benzamide | |
| 669 | N-cyclopropyl-N-(3-(4-(cyclopropylamino)-7-methylquinazolin-6- | 477.3 |
| | yl)benzyl)-3-phenylpropanamide | |
| 670 | N-cyclopropyl-N-(3-(4-(cyclopropylamino)-8-fluoroquinazolin-6- | 467.2 |
| | yl)benzyl)-2-phenylacetamide | |
| 671 | N-(2-fluorophenyl)-N-(3-(4-(methylamino)quinazolin-6- | 454.2 |
| | yl)benzyl)isoxazole-5-carboxamide | |
| 672 | N-(2-fluorophenyl)-N-(3-(4-(methylamino)quinazolin-6-yl)benzyl)- | 567.1 |
| | 3-(trifluoromethyl)benzole sulfonamide | |
| 673 | N-cyclopropyl-N-(3-(4-(2,4-dimethoxybenzylamino)quinazolin-6- | 525.3 |
| | yl)benzyl)pentanamide | |
| 674 | 3,5-dichloro-N-(2-fluoro-5-(4-(methylamino)quinazolin-6- | 469.1 |
| | yl)benzyl)-N-methylbenzamide | |
| 675 | N-cyclopropyl-N-(3-(4-(cyclopropylamino)-7-methylquinazolin-6- | 575.2 |
| | yl)benzyl)-2,3,4,5,6-pentafluorobenzole sulfonamide | |
| 676 | N-cyclopropyl-N-(3-(4-(cyclopropylamino)-7-methylquinazolin-6- | 517.2 |
| | yl)benzyl)-3-(trifluoromethyl)benzamide | |
| 677 | N-cyclopropyl-N-(3-(4-(cyclopropylamino)-8-fluoroquinazolin-6- | 443.2 |
| | yl)benzyl)furan-2-carboxamide | |
| 678 | N-cyclopropyl-N-(3-(4-(cyclopropylamino)-8-fluoroquinazolin-6- | 427.2 |
| | yl)benzyl)methane sulfonamide | |
| 679 | N-(2-fluorophenyl)-N-(3-(4-(methylamino)quinazolin-6- | 463.2 |
| | yl)benzyl)benzamide | |
| 680 | N-(2-fluorophenyl)-4-methoxy-N-(3-(4-(methylamino)quinazolin- | 529.2 |
| | 6-yl)benzyl)benzole sulfonamide | |
| 681 | N-cyclopropyl-N-(3-(4-(2,4-dimethoxybenzylamino)quinazolin-6- | 513.3 |
| | yl)benzyl)-2-methoxyacetamide | |
| 682 | N-cyclopropyl-N-(5-(4-(cyclopropylamino)-2-methylquinazolin-6- | 458.2 |
| | yl)-2-fluorobenzyl)isoxazole-5-carboxamide | |
| 683 | N-cyclopropyl-N-(3-(4-(cyclopropylamino)-7-methylquinazolin-6- | 536.2 |
| | yl)benzyl)quinoline-8-sulfonamide | |
| 684 | N-cyclopropyl-N-(3-(4-(cyclopropylamino)-7-methylquinazolin-6- | 517.2 |
| | yl)benzyl)-2-(trifluoromethyl)benzamide | |
| 685 | N-cyclopropyl-N-(3-(4-(cyclopropylamino)-8-fluoroquinazolin-6- | 459.2 |
| | yl)benzyl)thiophene-2-carboxamide | |
| 686 | N-cyclopropyl-N-(3-(4-(cyclopropylamino)-8-fluoroquinazolin-6- | 557.2 |
| | yl)benzyl)-3-(trifluoromethyl)benzole sulfonamide | |
| 687 | 3-fluoro-N-(2-fluorophenyl)-N-(3-(4-(methylamino)quinazolin-6- | 481.2 |
| | yl)benzyl)benzamide | |
| 688 | N-(3-(4-(methylamino)quinazolin-6-yl)benzyl)-N-(pyridin-2- | 451.2 |
| | ylmethyl)isoxazole-5-carboxamide | |
| 689 | N-cyclopropyl-N-(3-(4-(2,4-dimethoxybenzylamino)quinazolin-6- | 526.3 |
| | yl)benzyl)-2-(dimethylamino)acetamide | |
| 690 | N-cyclopropyl-N-(3-(4-(cyclopropylamino)-8-fluoroquinazolin-6- | 489.2 |
| | yl)benzyl)benzole sulfonamide | |
| 691 | N-cyclopropyl-N-(3-(4-(cyclopropylamino)-7-methylquinazolin-6- | 439.2 |
| | yl)benzyl)furan-2-carboxamide | |
| 692 | 3,5-dichloro-N-cyclopropyl-N-(3-(4-(cyclopropylamino)-7- | 517.2 |
| | methylquinazolin-6-yl)benzyl)benzamide | |
| 693 | N-cyclopropyl-N-(3-(4-(cyclopropylamino)-8-fluoroquinazolin-6- | 453.2 |
| | yl)benzyl)benzamide | |
| 694 | N-cyclopropyl-N-(3-(4-(cyclopropylamino)-8-fluoroquinazolin-6- | 519.2 |
| | yl)benzyl)-4-methoxybenzole sulfonamide | |
| 695 | 2,3,4,5,6-pentafluoro-N-(2-fluorophenyl)-N-(3-(4-(methylamino) | 553.1 |
| | quinazolin-6-yl)benzyl)benzamide | |
| 696 | N-cyclopropyl-N-(3-(4-(2,4-dimethoxybenzylamino)quinazolin-6- | 551.3 |
| | yl)benzyl)cyclohexane carboxamide | |
| 697 | N-cyclopropyl-N-(3-(4-(cyclopropylamino)-7-methylquinazolin-6- | 423.2 |
| | yl)benzyl)methane sulfonamide | |
| 698 | N-cyclopropyl-N-(3-(4-(cyclopropylamino)-7-methylquinazolin-6- | 455.2 |
| | yl)benzyl)thiophene-2-carboxamide | |
| 699 | N-cyclopropyl-N-(3-(4-(cyclopropylamino)-7-methylquinazolin-6- | 450.2 |
| | yl)benzyl)picolinamide | |
| 700 | N-cyclopropyl-N-(3-(4-(cyclopropylamino)-8-fluoroquinazolin-6- | 513.2 |
| | yl)benzyl)-3,4-dimethoxybenzamide | |
| 701 | N-cyclopropyl-N-(3-(4-(cyclopropylamino)-8-fluoroquinazolin-6- | 495.1 |
| | yl)benzyl)thiophene-2-sulfonamide | |
| 702 | N-cyclopropyl-N-(3-(4-(cyclopropylamino)-7-methylquinazolin-6- | 450.2 |
| | yl)benzyl)nicotinamide | |
| 703 | N-cyclopropyl-N-(3-(4-(cyclopropylamino)-8-fluoroquinazolin-6- | 481.2 |
| | yl)benzyl)-3-phenylpropanamide | |
| 704 | N-cyclopropyl-N-(3-(4-(2,4-dimethoxybenzylamino)quinazolin-6- | 559.3 |
| | yl)benzyl)-2-phenylacetamide | |
| 705 | N-cyclopropyl-N-(3-(4-(cyclopropylamino)-7-methylquinazolin-6- | 451.2 |
| | yl)benzyl)propane-2-sulfonamide | |
| 706 | N-cyclopropyl-N-(3-(4-(cyclopropylamino)-7-methylquinazolin-6- | 467.3 |
| | yl)benzyl)-1,3-dimethyl-1H-pyrazole-5-carboxamide | |
| 707 | N-cyclopropyl-N-(3-(4-(cyclopropylamino)-7-methylquinazolin-6- | 450.2 |
| | yl)benzyl)isonicotinamide | |
| 708 | N-cyclopropyl-N-(3-(4-(cyclopropylamino)-8-fluoroquinazolin-6- | 454.2 |
| | yl)benzyl)picolinamide | |
| 709 | N-cyclopropyl-N-(3-(4-(cyclopropylamino)-8-fluoroquinazolin-6- | 540.2 |
| | yl)benzyl)quinoline-8-sulfonamide | |
| 710 | N-cyclopropyl-N-(3-(4-(cyclopropylamino)-7-methylquinazolin-6- | 387.2 |
| | yl)benzyl)acetamide | |
| 711 | N-(2-fluorophenyl)-N-(3-(4-(methylamino)quinazolin-6- | 401.2 |
| | yl)benzyl)acetamide | |
| 712 | N-cyclopropyl-N-(3-(4-(2,4-dimethoxybenzylamino)quinazolin-6- | 573.3 |
| | yl)benzyl)-3-phenylpropanamide | |
| 713 | N-cyclopropyl-N-(3-(4-(cyclopropylamino)-7-methylquinazolin-6- | 499.2 |
| | yl)benzyl)-1-phenylmethane sulfonamide | |
| 714 | N-cyclopropyl-N-(3-(4-(cyclopropylamino)-7-methylquinazolin-6- | 449.2 |
| | yl)benzyl)benzamide | |
| 715 | N-cyclopropyl-N-(3-(4-(cyclopropylamino)-7-methylquinazolin-6- | 429.3 |
| | yl)benzyl)pentanamide | |
| 716 | N-cyclopropyl-N-(3-(4-(cyclopropylamino)-8-fluoroquinazolin-6- | 391.2 |
| | yl)benzyl)acetamide | |
| 717 | N-(2-fluorophenyl)-N-(3-(4-(methylamino)quinazolin-6- | 437.1 |
| | yl)benzyl)methane sulfonamide | |
| 718 | 2-(dimethylamino)-N-(2-fluoro-5-(4-(methylamino)quinazolin-6- | 382.2 |
| | yl)benzyl)-N-methylacetamide | |
| 719 | N-cyclopropyl-N-(3-(4-(cyclopropylamino)-7-methylquinazolin-6- | 429.3 |
| | yl)benzyl)pivalamide | |
| 720 | N-(3-(4-(methylamino)quinazolin-6-yl)benzyl)-N-(pyridin-2- | 528.2 |
| | ylmethyl)-2-(trifluoromethyl)benzamide | |
| 721 | N-cyclopropyl-N-(3-(4-(cyclopropylamino)-7-methylquinazolin-6- | 485.2 |
| | yl)benzyl)benzole sulfonamide | |
| 722 | N-cyclopropyl-N-(3-(4-(cyclopropylamino)-7-methylquinazolin-6- | 509.3 |
| | yl)benzyl)-3,4-dimethoxybenzamide | |
| 723 | N-cyclopropyl-N-(3-(4-(cyclopropylamino)-7-methylquinazolin-6- | 430.3 |
| | yl)benzyl)-2-(dimethylamino)acetamide | |
| 724 | N-cyclopropyl-N-(3-(4-(cyclopropylamino)-8-fluoroquinazolin-6- | 433.2 |
| | yl)benzyl)pivalamide | |
| 725 | 2,4-dichloro-N-(2-fluorophenyl)-N-(3-(4-(methylamino)quinazolin- | 567.1 |
| | 6-yl)benzyl)benzole sulfonamide | |
| 726 | N-cyclopropyl-2-(dimethylamino)-N-(3-(4-(3-methylbenzylamino) | 480.3 |
| | quinazolin-6-yl)benzyl)acetamide | |
| 727 | N-cyclopropyl-N-(3-(4-(cyclopropylamino)-8-fluoroquinazolin-6- | 471.2 |
| | yl)benzyl)-3-fluorobenzamide | |
| 728 | N-cyclopropyl-N-(3-(4-(cyclopropylamino)-8-fluoroquinazolin-6- | 521.2 |
| | yl)benzyl)-3-(trifluoromethyl)benzamide | |
| 729 | 3,5-dichloro-N-(2-fluorophenyl)-N-(3-(4-(methylamino)quinazolin- | 531.1 |
| | 6-yl)benzyl)benzamide | |
| 730 | N-cyclopropyl-N-(3-(4-(2,4-dimethoxybenzylamino)quinazolin-6- | 563.3 |
| | yl)benzyl)-1,3-dimethyl-1H-pyrazole-5-carboxamide | |
| 731 | N-cyclopropyl-N-((4-(4-(cyclopropylamino)quinazolin-6- | 472.2 |
| | yl)pyridin-2-yl)methyl)benzole sulfonamide | |
| 732 | N-(2-chloro-5-(4-(cyclopropylamino)quinazolin-6-yl)benzyl)-N- | 537.2 |
| | cyclopropyl-3-(trifluoromethyl)benzamide | |
| 733 | N-(2-chloro-5-(4-(cyclopropylamino)quinazolin-6-yl)benzyl)-N- | 450.2 |
| | cyclopropyl-2-(dimethylamino)acetamide | |
| 734 | N-(2-chloro-5-(4-(cyclopropylamino)quinazolin-6-yl)benzyl)-N- | 511.1 |
| | cyclopropylthiophene-2-sulfonamide | |
| 735 | N-cyclopropyl-N-((4-(4-(cyclopropylamino)quinazolin-6- | 522.2 |
| | yl)pyridin-2-yl)methyl)-2-fluoro-4-(trifluoromethyl)benzamide | |
| 736 | N-cyclopropyl-N-(3-(4-(cyclopropylamino)-8-fluoroquinazolin-6- | 454.2 |
| | yl)benzyl)isonicotinamide | |
| 737 | N-cyclopropyl-N-(3-(4-(cyclopropylamino)-7-methylquinazolin-6- | 491.2 |
| | yl)benzyl)thiophene-2-sulfonamide | |
| 738 | N-cyclopropyl-N-(3-(4-(cyclopropylamino)-8-fluoroquinazolin-6- | 539.2 |
| | yl)benzyl)-2-fluoro-4-(trifluoromethyl)benzamide | |
| 739 | N-cyclopropyl-N-((4-(4-(cyclopropylamino)quinazolin-6- | 417.2 |
| | yl)pyridin-2-yl)methyl)-2-(dimethylamino)acetamide | |
| 740 | N-(2-chloro-5-(4-(cyclopropylamino)quinazolin-6-yl)benzyl)-N- | 537.2 |
| | cyclopropyl-2-(trifluoromethyl)benzamide | |
| 741 | N-(2-chloro-5-(4-(cyclopropylamino)quinazolin-6-yl)benzyl)-N- | 475.2 |
| | cyclopropylcyclohexane carboxamide | |
| 742 | N-(2-chloro-5-(4-(cyclopropylamino)quinazolin-6-yl)benzyl)-N- | 556.2 |
| | cyclopropylquinoline-8-sulfonamide | |
| 743 | N-cyclopropyl-N-((4-(4-(cyclopropylamino)quinazolin-6- | 504.2 |
| | yl)pyridin-2-yl)methyl)-3-(trifluoromethyl)benzamide | |
| 744 | N-cyclopropyl-N-(3-(4-(cyclopropylamino)-8-fluoroquinazolin-6- | 421.2 |
| | yl)benzyl)-2-methoxyacetamide | |
| 745 | N-cyclopropyl-N-(3-(4-(cyclopropylamino)-7-methylquinazolin-6- | 440.2 |
| | yl)benzyl)isoxazole-5-carboxamide | |
| 746 | N-(2-chloro-5-(4-(cyclopropylamino)quinazolin-6-yl)benzyl)-N- | 459.2 |
| | cyclopropylfuran-2-carboxamide | |
| 747 | 3,5-dichloro-N-(2-chloro-5-(4-(cyclopropylamino)quinazolin-6- | 537.1 |
| | yl)benzyl)-N-cyclopropylbenzamide | |
| 748 | N-(2-chloro-5-(4-(cyclopropylamino)quinazolin-6-yl)benzyl)-N- | 483.2 |
| | cyclopropyl-2-phenylacetamide | |
| 749 | N-cyclopropyl-N-((4-(4-(cyclopropylamino)quinazolin-6- | 504.2 |
| | yl)pyridin-2-yl)methyl)-2-(trifluoromethyl)benzamide | |
| 750 | 3,5-dichloro-N-cyclopropyl-N-((4-(4- | 504.1 |
| | (cyclopropylamino)quinazolin-6-yl)pyridin-2-yl)methyl)benzamide | |
| 751 | N-cyclopropyl-N-(3-(4-(cyclopropylamino)-8-fluoroquinazolin-6- | 434.2 |
| | yl)benzyl)-2-(dimethylamino)acetamide | |
| 752 | N-cyclopropyl-N-(3-(4-(cyclopropylamino)-8-fluoroquinazolin-6- | 543.2 |
| | yl)benzyl)-2,3,4,5,6-pentafluorobenzamide | |
| 753 | N-cyclopropyl-N-(3-(4-(cyclopropylamino)-8-fluoroquinazolin-6- | 503.2 |
| | yl)benzyl)-1-phenylmethane sulfonamide | |
| 754 | N-(2-chloro-5-(4-(cyclopropylamino)quinazolin-6-yl)benzyl)-N- | 475.1 |
| | cyclopropylthiophene-2-carboxamide | |
| 755 | N-(2-chloro-5-(4-(cyclopropylamino)quinazolin-6-yl)benzyl)-N- | 470.2 |
| | cyclopropylpicolinamide | |
| 756 | N-(2-chloro-5-(4-(cyclopropylamino)quinazolin-6-yl)benzyl)-N- | 497.2 |
| | cyclopropyl-3-phenylpropanamide | |
| 757 | N-cyclopropyl-N-((4-(4-(cyclopropylamino)quinazolin-6- | 426.2 |
| | yl)pyridin-2-yl)methyl)furan-2-carboxamide | |
| 758 | N-cyclopropyl-N-((4-(4-(cyclopropylamino)quinazolin-6- | 437.2 |
| | yl)pyridin-2-yl)methyl)picolinamide | |
| 759 | N-cyclopropyl-N-(3-(4-(cyclopropylamino)-7-methylquinazolin-6- | 470.2 |
| | yl)benzyl)-2-methylthiazole-4-carboxamide | |
| 760 | N-cyclopropyl-N-(3-(4-(cyclopropylamino)-8-fluoroquinazolin-6- | 471.2 |
| | yl)benzyl)-1,3-dimethyl-1H-pyrazole-5-carboxamide | |
| 761 | N-cyclopropyl-N-(3-(4-(cyclopropylamino)-8-fluoroquinazolin-6- | 579.1 |
| | yl)benzyl)-2,3,4,5,6-pentafluorobenzole sulfonamide | |
| 762 | N-(2-chloro-5-(4-(cyclopropylamino)quinazolin-6-yl)benzyl)-N- | 460.2 |
| | cyclopropylisoxazole-5-carboxamide | |
| 763 | N-(2-chloro-5-(4-(cyclopropylamino)quinazolin-6-yl)benzyl)-N- | 470.2 |
| | cyclopropylisonicotinamide | |
| 764 | N-(2-chloro-5-(4-(cyclopropylamino)quinazolin-6-yl)benzyl)-N- | 443.1 |
| | cyclopropylmethane sulfonamide | |
| 765 | N-cyclopropyl-N-((4-(4-(cyclopropylamino)quinazolin-6- | 427.2 |
| | yl)pyridin-2-yl)methyl)isoxazole-5-carboxamide | |
| 766 | N-cyclopropyl-N-((4-(4-(cyclopropylamino)quinazolin-6- | 437.2 |
| | yl)pyridin-2-yl)methyl)nicotinamide | |
| 767 | N-(2-fluorophenyl)-N-(3-(4-(methylamino)quinazolin-6- | 453.2 |
| | yl)benzyl)furan-2-carboxamide | |
| 768 | N-cyclopropyl-N-(3-(4-(cyclopropylamino)-7-methylquinazolin-6- | 539.2 |
| | yl)benzyl)-2,3,4,5,6-pentafluorobenzamide | |
| 769 | N-(2-chloro-5-(4-(cyclopropylamino)quinazolin-6-yl)benzyl)-N- | 490.1 |
| | cyclopropyl-2-methylthiazole-4-carboxamide | |
| 770 | N-(2-chloro-5-(4-(cyclopropylamino)quinazolin-6-yl)benzyl)-N- | 470.2 |
| | cyclopropylnicotinamide | |
| 771 | N-(2-chloro-5-(4-(cyclopropylamino)quinazolin-6-yl)benzyl)-N- | 519.2 |
| | cyclopropyl-1-phenylmethane sulfonamide | |
| 772 | N-cyclopropyl-N-((4-(4-(cyclopropylamino)quinazolin-6- | 457.2 |
| | yl)pyridin-2-yl)methyl)-2-methylthiazole-4-carboxamide | |
| 773 | N-cyclopropyl-N-((4-(4-(cyclopropylamino)quinazolin-6- | 374.2 |
| | yl)pyridin-2-yl)methyl)acetamide | |
| 774 | 3,5-dichloro-N-cyclopropyl-N-(3-(4-(cyclopropylamino)-8- | 521.1 |
| | fluoroquinazolin-6-yl)benzyl)benzamide | |
| 775 | N-cyclopropyl-N-(3-(4-(cyclopropylamino)-8-fluoroquinazolin-6- | 444.2 |
| | yl)benzyl)isoxazole-5-carboxamide | |
| 776 | N-(2-chloro-5-(4-(cyclopropylamino)quinazolin-6-yl)benzyl)-N- | 487.2 |
| | cyclopropyl-1,3-dimethyl-1H-pyrazole-5-carboxamide | |
| 777 | N-(2-chloro-5-(4-(cyclopropylamino)quinazolin-6-yl)benzyl)-N- | 407.2 |
| | cyclopropylacetamide | |
| 778 | N-(2-chloro-5-(4-(cyclopropylamino)quinazolin-6-yl)benzyl)-N- | 573.1 |
| | cyclopropyl-3-(trifluoromethyl)benzole sulfonamide | |
| 779 | N-cyclopropyl-N-((4-(4-(cyclopropylamino)quinazolin-6- | 454.2 |
| | yl)pyridin-2-yl)methyl)-1,3-dimethyl-1H-pyrazole-5-carboxamide | |
| 780 | N-cyclopropyl-N-((4-(4-(cyclopropylamino)quinazolin-6- | 404.2 |
| | yl)pyridin-2-yl)methyl)-2-methoxyacetamide | |
| 781 | N-cyclopropyl-N-(3-(4-(cyclopropylamino)-8-fluoroquinazolin-6- | 454.2 |
| | yl)benzyl)nicotinamide | |
| 782 | 2,4-dichloro-N-cyclopropyl-N-(3-(4-(cyclopropylamino)-8- | 557.1 |
| | fluoroquinazolin-6-yl)benzyl)benzole sulfonamide | |
| 783 | N-(2-chloro-5-(4-(cyclopropylamino)quinazolin-6-yl)benzyl)-N- | 469.2 |
| | cyclopropylbenzamide | |
| 784 | N-(2-chloro-5-(4-(cyclopropylamino)quinazolin-6-yl)benzyl)-N- | 529.2 |
| | cyclopropyl-3,4-dimethoxybenzamide | |
| 785 | N-(2-chloro-5-(4-(cyclopropylamino)quinazolin-6-yl)benzyl)-N- | 449.2 |
| | cyclopropylpivalamide | |
| 786 | N-(2-chloro-5-(4-(cyclopropylamino)quinazolin-6-yl)benzyl)-N- | 535.2 |
| | cyclopropyl-4-methoxybenzole sulfonamide | |
| 787 | N-cyclopropyl-N-((4-(4-(cyclopropylamino)quinazolin-6- | 496.2 |
| | yl)pyridin-2-yl)methyl)-3,4-dimethoxybenzamide | |
| 788 | N-cyclopropyl-N-((4-(4-(cyclopropylamino)quinazolin-6- | 478.1 |
| | yl)pyridin-2-yl)methyl)thiophene-2-sulfonamide | |
| 789 | N-cyclopropyl-N-(3-(4-(cyclopropylamino)-8-fluoroquinazolin-6- | 521.2 |
| | yl)benzyl)-2-(trifluoromethyl)benzamide | |
| 790 | 2-(dimethylamino)-N-(3-(4-(methylamino)quinazolin-6-yl)benzyl)- | 441.2 |
| | N-(pyridin-2-ylmethyl)acetamide | |
| 791 | N-(2-chloro-5-(4-(cyclopropylamino)quinazolin-6-yl)benzyl)-N- | 487.2 |
| | cyclopropyl-3-fluorobenzamide | |
| 792 | N-(2-chloro-5-(4-(cyclopropylamino)quinazolin-6-yl)benzyl)-N- | 505.2 |
| | cyclopropyl-2,4-difluorobenzamide | |
| 793 | N-(2-chloro-5-(4-(cyclopropylamino)quinazolin-6-yl)benzyl)-N- | 449.2 |
| | cyclopropylpentanamide | |
| 794 | 2,4-dichloro-N-(2-chloro-5-(4-(cyclopropylamino)quinazolin-6- | 573.1 |
| | yl)benzyl)-N-cyclopropylbenzole sulfonamide | |
| 795 | N-cyclopropyl-N-((4-(4-(cyclopropylamino)quinazolin-6- | 454.2 |
| | yl)pyridin-2-yl)methyl)-3-fluorobenzamide | |
| 796 | 2,4-dichloro-N-cyclopropyl-N-((4-(4-(cyclopropylamino)- | 540.1 |
| | quinazolin-6-yl)pyridin-2-yl)methyl)benzole sulfonamide | |
| 797 | N-cyclopropyl-N-(3-(4-(cyclopropylamino)-7-methylquinazolin-6- | 417.2 |
| | yl)benzyl)-2-methoxyacetamide | |
| 798 | N-cyclopropyl-2-methoxy-N-(3-(4-(phenethylamino)quinazolin-6- | 467.2 |
| | yl)benzyl)acetamide | |
| 799 | N-(2-chloro-5-(4-(cyclopropylamino)quinazolin-6-yl)benzyl)-N- | 505.1 |
| | cyclopropylbenzole sulfonamide | |
| 800 | N-(2-chloro-5-(4-(cyclopropylamino)quinazolin-6-yl)benzyl)-N- | 555.2 |
| | cyclopropyl-2-fluoro-4-(trifluoromethyl)benzamide | |
| 801 | N-(2-chloro-5-(4-(cyclopropylamino)quinazolin-6-yl)benzyl)-N- | 437.2 |
| | cyclopropyl-2-methoxyacetamide | |
| 802 | N-(2-chloro-5-(4-(cyclopropylamino)quinazolin-6-yl)benzyl)-N- | 595.1 |
| | cyclopropyl-2,3,4,5,6-pentafluorobenzole sulfonamide | |
| 803 | N-cyclopropyl-N-((4-(4-(cyclopropylamino)quinazolin-6- | 526.2 |
| | yl)pyridin-2-yl)methyl)-2,3,4,5,6-pentafluorobenzamide | |
| 804 | N-cyclopropyl-N-((4-(4-(cyclopropylamino)quinazolin-6- | 438.2 |
| | yl)pyridin-2-yl)methyl)propane-2-sulfonamide | |
| 805 | N-cyclopropyl-N-((4-(4-(cyclopropylamino)quinazolin-6- | 486.2 |
| | yl)pyridin-2-yl)methyl)-1-phenylmethane sulfonamide | |
| 806 | N-cyclopropyl-N-((4-(4-(cyclopropylamino)quinazolin-6- | 502.2 |
| | yl)pyridin-2-yl)methyl)-4-methoxybenzole sulfonamide | |
| 807 | N-(5-(4-(tert-butylamino)quinazolin-6-yl)-2-fluorobenzyl)-N- | 459.2 |
| | cyclopropylfuran-2-carboxamide | |
| 808 | N-(5-(4-(tert-butylamino)quinazolin-6-yl)-2-fluorobenzyl)-N- | 537.2 |
| | cyclopropyl-3-(trifluoromethyl)benzamide | |
| 809 | N-(5-(4-(tert-butylamino)quinazolin-6-yl)-2-fluorobenzyl)-N- | 475.3 |
| | cyclopropylcyclohexane carboxamide | |
| 810 | N-(5-(4-(tert-butylamino)quinazolin-6-yl)-2-fluorobenzyl)-N- | 511.2 |
| | cyclopropylthiophene-2-sulfonamide | |
| 811 | N-cyclopropyl-N-((4-(4-(cyclopropylamino)quinazolin-6- | 442.2 |
| | yl)pyridin-2-yl)methyl)thiophene-2-carboxamide | |
| 812 | N-cyclopropyl-N-((4-(4-(cyclopropylamino)quinazolin-6- | 523.2 |
| | yl)pyridin-2-yl)methyl)quinoline-8-sulfonamide | |
| 813 | N-cyclopropyl-N-((4-(4-(cyclopropylamino)quinazolin-6- | 436.2 |
| | yl)pyridin-2-yl)methyl)benzamide | |
| 814 | N-(5-(4-(tert-butylamino)quinazolin-6-yl)-2-fluorobenzyl)-N- | 537.2 |
| | cyclopropyl-2-(trifluoromethyl)benzamide | |
| 815 | N-(5-(4-(tert-butylamino)quinazolin-6-yl)-2-fluorobenzyl)-N- | 483.3 |
| | cyclopropyl-2-phenylacetamide | |
| 816 | N-(5-(4-(tert-butylamino)quinazolin-6-yl)-2-fluorobenzyl)-N- | 556.2 |
| | cyclopropylquinoline-8-sulfonamide | |
| 817 | N-cyclopropyl-N-((4-(4-(cyclopropylamino)quinazolin-6- | 472.2 |
| | yl)pyridin-2-yl)methyl)-2,4-difluorobenzamide | |
| 818 | N-cyclopropyl-N-(3-(4-(phenethylamino)quinazolin-6- | 501.2 |
| | yl)benzyl)propane-2-sulfonamide | |
| 819 | N-(5-(4-(tert-butylamino)quinzolin-6-yl)-2-fluorobenzyl)-N- | 475.2 |
| | cyclopropylthiophene-2-carboxamide | |
| 820 | N-(5-(4-(tert-butylamino)quinazolin-6-yl)-2-fluorobenzyl)-3,5- | 537.2 |
| | dichloro-N-cyclopropylbenzamide | |
| 821 | N-(5-(4-(tert-butylamino)quinazolin-6-yl)-2-fluorobenzyl)-N- | 497.3 |
| | cyclopropyl-3-phenylpropanamide | |
| 822 | N-(5-(4-(tert-butylamino)quinazolin-6-yl)-2-fluorobenzyl)-N- | 471.2 |
| | cyclopropylpropane-2-sulfonamide | |
| 823 | N-cyclopropyl-N-((4-(4-(cyclopropylamino)quinazolin-6- | 437.2 |
| | yl)pyridin-2-yl)methyl)isonicotinamide | |
| 824 | N-cyclopropyl-N-(3-(4-(cyclopropylamino)-8-fluoroquinazolin-6- | 455.2 |
| | yl)benzyl)propane-2-sulfonamide | |
| 825 | N-(5-(4-(tert-butylamino)quinazolin-6-yl)-2-fluorobenzyl)-N- | 490.2 |
| | cyclopropyl-2-methylthiazole-4-carboxamide | |
| 826 | N-(5-(4-(tert-butylamino)quinazolin-6-yl)-2-fluorobenzyl)-N- | 470.2 |
| | cyclopropylpicolinamide | |
| 827 | N-(5-(4-(tert-butylamino)quinazolin-6-yl)-2-fluorobenzyl)-N- | 443.2 |
| | cyclopropylmethane sulfonamide | |
| 828 | N-cyclopropyl-N-((4-(4-(cyclopropylamino)quinazolin-6- | 416.2 |
| | yl)pyridin-2-yl)methyl)pivalamide | |
| 829 | N-cyclopropyl-N-((4-(4-(cyclopropylamino)quinazolin-6- | 416.2 |
| | yl)pyridin-2-yl)methyl)pentanamide | |
| 830 | N-cyclopropyl-N-(3-(4-(cyclopropylamino)-8-fluoroquinazolin-6- | 474.2 |
| | yl)benzyl)-2-methylthiazole-4-carboxamide | |
| 831 | N-(5-(4-(tert-butylamino)quinazolin-6-yl)-2-fluorobenzyl)-N- | 487.3 |
| | cyclopropyl-1,3-dimethyl-1H-pyrazole-5-carboxamide | |
| 832 | N-(5-(4-(tert-butylamino)quinazolin-6-yl)-2-fluorobenzyl)-N- | 470.2 |
| | cyclopropylisonicotinamide | |
| 833 | N-(5-(4-(tert-butylamino)quinazolin-6-yl)-2-fluorobenzyl)-N | 519.2 |
| | cyclopropyl-1-phenylmethane sulfonamide | |
| 834 | N-(5-(4-(tert-butylamino)quinazolin-6-yl)-2-fluorobenzyl)-N- | 460.2 |
| | cyclopropylisoxazole-5-carboxamide | |
| 835 | N-cyclopropyl-N-((4-(4-(cyclopropylamino)quinazolin-6- | 442.3 |
| | yl)pyridin-2-yl)methyl)cyclohexane carboxamide | |
| 836 | N-cyclopropyl-N-(3-(4-(2,4-dimethoxybenzylamino)quinazolin-6- | 547.2 |
| | yl)benzyl)propane-2-sulfonamide | |
| 837 | N-(5-(4-(tert-butylamino)quinazolin-6-yl)-2-fluorobenzyl)-N- | 469.2 |
| | cyclopropylbenzamide | |
| 838 | N-(5-(4-(tert-butylamino)quinazolin-6-yl)-2-fluorobenzyl)-N- | 470.2 |
| | cyclopropylnicotinamide | |
| 839 | N-(5-(4-(tert-butylamino)quinazolin-6-yl)-2-fluorobenzyl)-N- | 505.2 |
| | cyclopropylbenzole sulfonamide | |
| 840 | N-(5-(4-(tert-butylamino)quinazolin-6-yl)-2-fluorobenzyl)-N- | 559.2 |
| | cyclopropyl-2,3,4,5,6-pentafluorobenzamide | |
| 841 | N-cyclopropyl-N-((4-(4-(cyclopropylamino)quinazolin-6- | 450.2 |
| | yl)pyridin-2-yl)methyl)-2-phenylacetamide | |
| 842 | N-cyclopropyl-N-(3-(4-(2,4-dimethoxybenzylamino)quinazolin-6- | 671.2 |
| | yl)benzyl)-2,3,4,5,6-pentafluorobenzole sulfonamide | |
| 843 | N-(5-(4-(tert-butylamino)quinazolin-6-yl)-2-fluorobenzyl)-N- | 529.3 |
| | cyclopropyl-3,4-dimethoxybenzamide | |
| 844 | N-(5-(4-(tert-butylamino)quinazolin-6-yl)-2-fluorobenzyl)-N- | 407.2 |
| | cyclopropylacetamide | |
| 845 | N-(5-(4-(tert-butylamino)quinazolin-6-yl)-2-fluorobenzyl)-N- | 573.2 |
| | cyclopropyl-3-(trifluoromethyl)benzole sulfonamide | |
| 846 | N-(5-(4-(tert-butylamino)quinazolin-6-yl)-2-fluorobenzyl)-N- | 437.2 |
| | cyclopropyl-2-methoxyacetamide | |
| 847 | N-cyclopropyl-N-((4-(4-(cyclopropylamino)quinazolin-6- | 464.2 |
| | yl)pyridin-2-yl)methyl)-3-phenylpropanamide | |
| 848 | N-cyclopropyl-N-((4-(4-(cyclopropylamino)quinazolin-6- | 562.1 |
| | yl)pyridin-2-yl)methyl)-2,3,4,5,6-pentafluorobenzole sulfonamide | |
| 849 | N-(5-(4-(tert-butylamino)quinazolin-6-yl)-2-fluorobenzyl)-N- | 487.2 |
| | cyclopropyl-3-fluorobenzamide | |
| 850 | N-(5-(4-(tert-butylamino)quinazolin-6-yl)-2-fluorobenzyl)-N- | 449.3 |
| | cyclopropylpivalamide | |
| 851 | N-(5-(4-(tert-butylamino)quinazolin-6-yl)-2-fluorobenzyl)-N- | 535.2 |
| | cyclopropyl-4-methoxybenzole sulfonamide | |
| 852 | N-(2-fluorophenyl)-N-(3-(4-(methylamino)quinazolin-6- | 499.2 |
| | yl)benzyl)benzole sulfonamide | |
| 853 | N-cyclopropyl-N-((4-(4-(cyclopropylamino)quinazolin-6- | 410.2 |
| | yl)pyridin-2-yl)methyl)methane sulfonamide | |
| 854 | N-(2-chloro-5-(4-(cyclopropylamino)quinazolin-6-yl)benzyl)-N- | 471.2 |
| | cyclopropylpropane-2-sulfonamide | |
| 855 | N-(5-(4-(tert-butylamino)quinazolin-6-yl)-2-fluorobenzyl)-N- | 505.2 |
| | cyclopropyl-2,4-difluorobenzamide | |
| 856 | N-(5-(4-(tert-butylamino)quinazolin-6-yl)-2-fluorobenzyl)-N- | 449.3 |
| | cyclopropylpentanamide | |
| 857 | N-(5-(4-(tert-butylamino)quinazolin-6-yl)-2-fluorobenzyl)-2,4- | 573.1 |
| | dichloro-N-cyclopropylbenzole sulfonamide | |
| 858 | N-cyclopropyl-N-((4-(4-(cyclopropylamino)quinazolin-6- | 540.2 |
| | yl)pyridin-2-yl)methyl)-3-(trifluoromethyl)benzole sulfonamide | |
| 859 | N-(2-chloro-5-(4-(cyclopropylamino)quinazolin-6-yl)benzyl)-N- | 559.1 |
| | cyclopropyl-2,3,4,5,6-pentafluorobenzamide | |
| 860 | N-(5-(4-(tert-butylamino)quinazolin-6-yl)-2-fluorobenzyl)-N- | 555.2 |
| | cyclopropyl-2-fluoro-4-(trifluoromethyl)benzamide | |
| 861 | N-(5-(4-(tert-butylamino)quinazolin-6-yl)-2-fluorobenzyl)-N- | 450.3 |
| | cyclopropyl-2-(dimethylamino)acetamide | |
| 862 | N-(5-(4-(tert-butylamino)quinazolin-6-yl)-2-fluorobenzyl)-N- | 595.2 |
| | cyclopropyl-2,3,4,5,6-pentafluorobenzole sulfonamide | |
| 866 | 1-(3-(4-(neopentylamino)quinazolin-6-yl)benzyl)pyrrolidine-2,5- | 403.2 |
| | dione | |
| 867 | 1-(3-(4-(2-methoxyethylamino)quinazolin-6-yl)benzyl)pyrrolidine- | 391.2 |
| | 2,5-dione hydrochloride | |
| 868 | N-cyclopropyl-N-(3-(4-(cyclopropylamino)quinazolin-6- | |
| | yl)benzyl)-1-methylpiperidine-4-carboxamide | |
| 869 | 1-amino-N-(3-(4-(cyclopropylamino)quinazolin-6- | 374.2 |
| | yl)benzyl)cyclopropane carboxamide dihydrochloride | |
| 870 | N-cyclopropyl-N-(5-(4-(cyclopropylamino)quinazolin-6-yl)-2- | 445.2 |
| | fluorobenzyl)-2,2,2-trifluoroacetamide | |
| 871 | 1-(3-(4-morpholinoquinazolin-6-yl)benzyl)pyrrolidine-2,5-dione | 403.2 |
| 872 | 1-(3-(4-(cyclobutylamino)quinazolin-6-yl)benzyl)pyrrolidine-2,5- | 387.2 |
| | dione hydrochloride | |
| 873 | 1-(3-(4-(isopropylamino)quinazolin-6-yl)benzyl)pyrrolidine-2,5- | 375.2 |
| | dione hydrochloride | |
| 874 | N-(3-(4-(cyclopropylamino)quinazolin-6-yl)benzyl)-1-pentane | |
| | amidocyclopropane carboxamide | |
| 875 | ethylcyclopropyl(3-(4-(cyclopropylamino)quinazolin-6- | 403.2 |
| | yl)benzyl)carbamate | |
| 876 | 6-(3-((1H-pyrrol-1-yl)methyl)phenyl)-N-cyclopropylquinazoline-4- | 341.2 |
| | amine | |
| 877 | 1-(3-(4-(cyclopropylamino)quinazolin-6-yl)benzyl)-3- | 374.2 |
| | methylimidazolidine-2-one | |
| 878 | N-cyclopropyl-6-(3-((methyl(pyridin-2-yl)amino)methyl)phenyl) | 382.2 |
| | quinazoline-4-amine | |
| 879 | N-acetyl-N-(3-(4-(cyclopropylamino)quinazolin-6- | 375.2 |
| | yl)benzyl)acetamide | |
| 880 | 1-(3-(4-(pyrrolidin-1-yl)quinazolin-6-yl)benzyl)pyrrolidine-2,5- | 387.2 |
| | dione | |
| 881 | 1-(3-(8-chloro-4-(cyclopropylamino)quinazolin-6- | 407.1 |
| | yl)benzyl)pyrrolidine-2,5-dione | |
| 882 | 1-(3-(4-(cyclopropylamino)-7-fluoroquinazolin-6- | 391.2 |
| | yl)benzyl)pyrrolidine-2,5-dione | |
| 883 | 1-(3-(4-(cyclopropylamino)-8-(trifluoromethyl)quinazolin-6- | 441.2 |
| | yl)benzyl)pyrrolidine-2,5-dione | |
| 884 | 1-(3-(4-(cyclopropylamino)-7-methylquinazolin-6- | 387.2 |
| | yl)benzyl)pyrrolidine-2,5-dione | |
| 885 | 1-(3-(4-(cyclopropylamino)quinazolin-6-yl)benzyl)-5- | 373.2 |
| | methylpyrrolidine-2-one | |
| 886 | N-allyl-N-(3-(4-(cyclopropylamino)quinazolin-6- | 373.2 |
| | yl)benzyl)acetamide | |
| 887 | N-(3-(4-(cyclopropylamino)quinazolin-6-yl)benzyl)-N- | 375.2 |
| | propylacetamide hydrochloride | |
| 888 | N-cyclopropyl-6-(3-((cyclopropyl(pyridin-2- | 408.2 |
| | yl)amino)methyl)phenyl) quinazoline-4-amine | |
| 889 | 1-(3-(4-(cyclopropylamino)quinazolin-6-yl)-5- | 391.2 |
| | fluorobenzyl)pyrrolidine-2,5-dione | |
| 890 | N-cyclopropyl-N-((6-(4-(cyclopropylamino)quinazolin-6- | 374.2 |
| | yl)pyridin-2-yl)methyl)acetamide | |
| 891 | N-cyclopropyl-6-(3-((cyclopropyl(methyl)amino)methyl)phenyl) | 345.2 |
| | quinazoline-4-amine | |
| 892 | N-cyclopropyl-N-(3-(4-(cyclopropylamino)quinazolin-6-yl)benzyl) | |
| | thiazole-2-amine | |
| 893 | N-cyclobutyl-N-(3-(4-(cyclopropylamino)quinazolin-6-yl)benzyl) | 387.2 |
| | acetamide | |
| 894 | N-(3-(4-(cyclopropylamino)quinazolin-6-yl)benzyl)-N- | 361.2 |
| | ethylacetamide | |
| 895 | 1-(3-(4-(cyclopropylamino)quinazolin-6-yl)-4- | 391.2 |
| | fluorobenzyl)pyrrolidine-2,5-dione | |
| 896 | N-cyclopropyl-N-(3-(4-(cyclopropylamino)quinazolin-6-yl)-4- | 391.2 |
| | fluorobenzyl)acetamide | |
| 897 | N-cyclopropyl-N-(3-(4-(cyclopropylamino)quinazolin-6-yl)-5- | 391.2 |
| | fluorobenzyl)acetamide | |
| 898 | methylcyclopropyl(3-(4-(cyclopropylamino)quinazolin-6- | 389.2 |
| | yl)benzyl) carbamate | |
| 899 | 1-(3-(4-(cyclopropylamino)-7-methoxyquinazolin-6-yl)benzyl) | 403.2 |
| | pyrrolidine-2,5-dione | |
| 900 | 2-cyano-N-cyclopropyl-N-(3-(4-(cyclopropylamino)quinazolin-6- | 416.2 |
| | yl)-4-fluorobenzyl)acetamide | |
| 901 | 1-(3-(4-(cyclopropyl(methyl)amino)quinazolin-6- | 387.2 |
| | yl)benzyl)pyrrolidine-2,5-dione | |
| 902 | 2-cyano-N-cyclopropyl-N-(3-(4-(cyclopropylamino)quinazolin-6- | 416.2 |
| | yl)-5-fluorobenzyl)acetamide | |
| 903 | N-cyclopropyl-N-((4-(4-(cyclopropylamino)quinazolin-6- | 380.2 |
| | yl)thiazole-2-yl)methyl)acetamide | |
| 904 | 1-(3-(4-(cyclopropylamino)-5-fluoroquinazolin-6- | 391.2 |
| | yl)benzyl)pyrrolidine-2,5-dione | |
| 905 | 2-cyano-N-cyclopropyl-N-(5-(4-(cyclopropylamino)-7- | 434.2 |
| | fluoroquinazolin-6-yl)-2-fluorobenzyl)acetamide | |
| 906 | 2-cyano-N-cyclopropyl-N-(3-(4-(cyclopropylamino)-7- | 416.2 |
| | fluoroquinazolin-6-yl)benzyl)acetamide | |
| 907 | 1-(3-(4-(cyclopropylamino)-8-fluoroquinazolin-6- | 391.2 |
| | yl)benzyl)pyrrolidine-2,5-dione | |
| 908 | N-cyclopropyl-6-(3-((cyclopropyl(phenyl)amino)methyl)phenyl) | 407.2 |
| | quinazoline-4-amine | |
| 909 | 1-((4-(4-(cyclopropylamino)quinazolin-6-yl)thiophen-2-yl)methyl) | 379.1 |
| | pyrrolidine-2,5-dione | |
| 910 | 1-(3-(4-(cyclopropylamino)-5,7-difluoroquinazolin-6-yl)benzyl) | 409.1 |
| | pyrrolidine-2,5-dione | |
| 911 | N-cyclopropyl-N-(3-(4-(cyclopropylamino)quinazolin-6-yl)-2- | 387.2 |
| | methylbenzyl)acetamide | |
| 912 | N-cyclopropyl-N-(5-(4-(cyclopropylmethyl)quinazolin-6-yl)-2- | 387.2 |
| | methylbenzyl)acetamide | |

### Example 868

1H NMR (600 MHz, DMSO-d₆) δ ppm 0.68 (m, 2H) 0.82 (m, 6H) 1.58-1.76 (m, 4H) 1.83-1.97 (m, 2H) 2.17 (s, 3H) 2.73 (br. s., 1H) 2.84 (d, J=10.58 Hz, 2H) 3.06 (br. s., 2H) 4.62 (s, 2H) 7.17 (d, J=7.55 Hz, 1H) 7.43-7.50 (m, 1H) 7.54 (br. s., 1H) 7.67 (d, J=7.55 Hz, 1H) 7.76 (d, J=8.31 Hz, 1H) 8.02 (d, J=8.31 Hz, 1H) 8.37 (br. s., 1H) 8.51 (m, 2H)

### Example 874

1H NMR (600 MHz, DMSO-d₆) δ ppm 0.60-0.65 (m, 2H) 0.75 (t, J=7.18 Hz, 3H) 0.80-0.85 (m, 2H) 0.87 (m, 2H) 1.21 (m, 2H) 1.30 (q, J=3.78 Hz, 2H) 1.47 (m, 2H) 2.17 (t, J=7.55 Hz, 2H) 2.98-3.09 (m, 1H) 4.41 (d, J=6.04 Hz, 2H) 7.27 (d, J=7.55 Hz, 1H) 7.44 (t, J=7.93 Hz, 1H) 7.67-7.73 (m, 2H) 7.76 (d, J=9.06 Hz, 1H) 8.17 (dd, J=8.69, 1.89 Hz, 1H) 8.28 (t, J=6.04 Hz, 1H) 8.51 (s, 1H) 8.53 (s, 2H) 8.59 (s, 1H)

### Example 892

1H NMR (600 MHz, DMSO-d₆) δ ppm 0.66-0.70 (m, 2H) 0.79-0.89 (m, 6H) 2.63 (ddd, J=6.61, 3.21, 3.02 Hz, 1H) 3.06 (td, J=7.18, 3.78 Hz, 1H) 4.88 (s, 2H) 6.82 (d, J=3.78 Hz, 1H) 7.21 (d, J=3.78 Hz, 1H) 7.26 (d, J=7.55 Hz, 1H) 7.45 (t, J=7.55 Hz, 1H) 7.64-7.71 (m, 2H) 7.75 (d, J=8.31 Hz, 1H) 8.00 (dd, J=8.69, 1.89 Hz, 1H) 8.37 (d, J=3.02 Hz, 1H) 8.50 (d, J=1.51 Hz, 1H) 8.52 (s, 1H)

### Example 194

1H NMR (300 MHz, DMSO-d₆) δ ppm 0.63-0.72 (m, 2H) 0.78-0.91 (m, 2H) 1.80-1.97 (m, 2H) 2.70 (m, 4H) 2.96-3.14 (m, 1H) 4.94 (s, 2H) 7.20 (d, J=7.91 Hz, 1H) 7.44 (t, J=7.72 Hz, 1H) 7.58-7.71 (m, 2H) 7.77 (d, J=8.67 Hz, 1H) 8.03 (dd, J=8.67, 1.88 Hz, 1H) 8.43 (d, J=3.01 Hz, 1H) 8.51 (s, 1H) 8.54 (s, 1H)

### Example 206

1H NMR (600 MHz, DMSO-d₆) δ ppm 0.93-1.03 (m, 4H) 2.15-2.30 (m, 2H) 3.16 (t, J=6.80 Hz, 2H) 3.27 (t, J=7.55 Hz, 2H) 3.34-3.41 (m, 1H) 4.21 (s, 2H) 7.45 (d, J=7.55 Hz, 1H) 7.52-7.62 (m, 1H) 7.80-7.88 (m, 2H) 7.96 (d, J=9.06 Hz, 1H) 8.36 (d, J=8.31 Hz, 1H) 8.98 (d, J=30.21 Hz, 2H) 9.82-10.09 (m, 1H) 10.48 (br. s., 1H)

### Example 224

1H NMR (600 MHz, DMSO-d₆) δ ppm 0.83-1.03 (m, 7H) 2.02-2.13 (m, 2H) 2.17-2.24 (m, 1H) 2.25-2.34 (m, 2H) 3.36-3.45 (m, 1H) 4.38 (d, J=6.04 Hz, 2H) 7.35 (d, J=8.31 Hz, 1H) 7.52 (t, J=7.55 Hz, 1H) 7.63-7.78 (m, 2H) 7.91 (d, J=9.06 Hz, 1H) 8.31 (d, J=9.07 Hz, 1H) 8.44 (t, J=5.67 Hz, 1H) 8.86 (br. s., 1H) 8.94 (s, 1H) 10.15 (s, 1H)

### Example 228

1H NMR (600 MHz, DMSO-d₆) δ ppm 0.93-1.01 (m, 4H) 1.19-1.34 (m, 4H) 1.69-1.78 (m, 2H) 1.86-1.94 (m, 1H) 1.95-2.01 (m, 1H) 3.38-3.44 (m, 3H) 4.31 (m, 1H) 4.42 (dd, J=15.49, 6.42 Hz, 1H) 7.32 (d, J=7.55 Hz, 1H) 7.48 (t, J=7.55 Hz, 1H) 7.66-7.78 (m, 2H) 7.95 (dd, J=8.69, 3.40 Hz, 1H) 8.35 (d, J=8.31 Hz, 1H) 8.42 (t, J=5.67 Hz, 1H) 8.80-9.02 (m, 2H) 10.22 (br. s., 1H)

### Example 229

1H NMR (600 MHz, DMSO-d₆) δ ppm 0.90-1.02 (m, 4H) 2.65 (dd, J=16.62, 5.29 Hz, 1H) 2.99 (dd, J=15.49, 9.44 Hz, 0.5H) 3.13 (dd, J=16.62, 10.58 Hz, 0.5H) 4.04-4.13 (m, 1H) 4.29-4.42 (m, 2H) 7.16-7.26 (m, 2H) 7.26-7.34 (m, 3H) 7.37 (d, J=7.55 Hz, 1H) 7.40-7.50 (m, 1H) 7.56-7.62 (s, 0.5H) 7.68-7.79 (m, 1.5H) 7.91-7.98 (m, 1H) 8.16 (d, J=8.31 Hz, 0.5H) 8.33 (d, J=9.06 Hz, 0.5H) 8.51-8.58 (m, 0.5H) 8.68-8.77 (m, 0.5H) 8.85-9.00 (m, 2H) 10.30 (br. s., 1H)

### Example 234

1H NMR (600 MHz, DMSO-d₆) δ ppm 0.63-0.73 (m, 6H) 0.82-0.88 (m, 2H) 2.51-2.55 (m, 1H) 3.03 (s, 3H) 3.04-3.12 (m, 1H) 4.50 (s, 2H) 7.81 (d, J=8.31 Hz, 1H) 8.03-8.14 (m, 2H) 8.41 (d, J=3.02 Hz, 1H) 8.55 (s, 1H) 8.61 (dd, J=14.35, 1.51 Hz, 2H) 8.99 (d, J=2.27 Hz, 1H)

### Example 240

1H NMR (600 MHz, DMSO-d₆) δ ppm 0.65-0.71 (m, 2H) 0.79-0.88 (m, 6H) 2.21 (s, 3H) 2.72-2.80 (m, 1H) 3.01-3.11 (m, 1H) 4.63 (s, 2H) 7.80 (d, J=8.31 Hz, 1H) 7.96 (br. s., 1H) 8.10 (dd, J=8.31, 1.51 Hz, 1H) 8.39 (d, J=3.78 Hz, 1H) 8.46 (d, J=1.51 Hz, 1H) 8.54 (s, 1H) 8.60 (d, J=1.51 Hz, 1H) 8.93 (d, J=1.51 Hz, 1H)

### Example 292

1H NMR (600 MHz, DMSO-d₆) δ ppm 0.65-0.71 (m, 2H) 0.81-0.88 (m, 2H) 2.91 (t, J=8.31 Hz, 2H) 3.01-3.10 (m, 1H) 3.25-3.35 (m, 2H) 4.36 (s, 2H) 6.59 (t, J=7.18 Hz, 1H) 6.63 (d, J=7.55 Hz, 1H) 6.99 (t, J=7.55 Hz, 1H) 7.05 (d, J=6.80 Hz, 1H) 7.40 (d, J=7.55 Hz, 1H) 7.50 (t, J=7.55 Hz, 1H) 7.69-7.80 (m, 3H) 8.07 (dd, J=8.31, 1.51 Hz, 1H) 8.39 (d, J=2.27 Hz, 1H) 8.53 (s, 2H)

### Example 296

1H NMR (600 MHz, DMSO-d₆) δ ppm 0.59-0.75 (m, 2H) 0.78-0.89 (m, 2H) 2.19 (s, 6H) 3.00-3.10 (m, 1H) 3.49 (s, 2H) 7.33 (d, J=7.55 Hz, 1H) 7.47 (t, J=7.93 Hz, 1H) 7.69 (br. s., 2H) 7.76 (d, J=9.06 Hz, 1H) 8.07 (d, J=8.31 Hz, 1H) 8.39 (br. s., 1H) 8.48-8.56 (m, 2H)

### Example 297

1H NMR (600 MHz, DMSO-d₆) δ ppm 0.93-0.99 (m, 2H) 1.02-1.07 (m, 2H) 3.43-3.51 (m, 1H) 4.16 (q, J=5.79 Hz, 2H) 7.50-7.57 (m, 1H) 7.60 (t, J=7.55 Hz, 1H) 7.88-7.99 (m, 2H) 8.25 (s, 1H) 8.42 (d, J=9.06 Hz, 1H) 8.51 (br. s., 3H) 8.95 (s, 1H) 9.19 (s, 1H) 10.36 (br. s., 2H)

### Example 306

1H NMR (600 MHz, DMSO-d₆) δ ppm 0.23-0.30 (m, 2H) 0.33-0.41 (m, 2H) 0.65-0.72 (m, 2H) 0.78-0.88 (m, 2H) 1.93-2.15 (m, 1H) 2.40 (s, 3H) 2.98-3.13 (m, 1H) 3.78 (s, 2H) 7.17 (s, 1H) 7.46 (s, 1H) 7.52 (s, 1H) 7.74 (d, J=8.31 Hz, 1H) 8.05 (dd, J=8.69, 1.89 Hz, 1H) 8.37 (d, J=3.02 Hz, 1H) 8.48 (d, J=1.51 Hz, 1H) 8.52 (s, 1H)

### Example 308

1H NMR (600 MHz, DMSO-d₆) δ ppm 0.58-0.73 (m, 2H) 0.78-0.91 (m, 2H) 3.01-3.10 (m, 1H) 3.74 (s, 2H) 3.80 (s, 2H) 7.18-7.26 (m, 1H) 7.32 (t, J=7.55 Hz, 2H) 7.35-7.42 (m, 3H) 7.47 (t, J=7.55 Hz, 1H) 7.69 (d, J=8.31 Hz, 1H) 7.73-7.80 (m, 2H) 8.08 (dd, J=8.69, 1.89 Hz, 1H) 8.39 (d, J=3.02 Hz, 1H) 8.53 (s, 2H)

### Example 311

### Purity Approx. 60%

1H NMR (600 MHz, DMSO-d₆) δ ppm 0.27-0.44 (m, 1H) 0.52-0.59 (m, 1H) 0.84-0.94 (m, 1H) 0.94-1.03 (m, 1H) 1.90 (s, 3H) 2.35 (s, 3H) 3.32-3.54 (m, 1H) 4.31-4.43 (m, 2H) 7.27-7.40 (m, 1H) 7.42-7.56 (m, 1H) 7.61-7.70 (m, 1H) 7.72-7.90 (m, 1H) 7.93-8.07 (m, 1H) 8.09-8.21 (m, 1H) 8.43 (br. m., 2H) 8.97 (d, J=20.40 Hz, 0.5H) 9.25 (s, 0.5H) 10.40 (s, 1H)

### Example 315

1H NMR (600 MHz, DMSO-d₆) δ ppm 0.63-0.77 (m, 2H) 0.79-0.89 (m, 2H) 3.02-3.11 (m, 1H) 3.48 (t, J=7.93 Hz, 2H) 4.22-4.34 (m, 2H) 4.45 (s, 2H) 7.33 (d, J=7.55 Hz, 1H) 7.54 (t, J=7.55 Hz, 1H) 7.68 (s, 1H) 7.77 (m, 2H) 8.09 (d, J=8.31 Hz, 1H) 8.47 (br. s., 1H) 8.54 (m, 2H)

### Example 868

1H NMR (600 MHz, DMSO-d₆) δ ppm 0.68 (m, 2H) 0.82 (m, 6H) 1.58-1.76 (m, 4H) 1.83-1.97 (m, 2H) 2.17 (s, 3H) 2.73 (br. s., 1H) 2.84 (d, J=10.58 Hz, 2H) 3.06 (br. s., 2H) 4.62 (s, 2H) 7.17 (d, J=7.55 Hz, 1H) 7.43-7.50 (m, 1H) 7.54 (br. s., 1H) 7.67 (d, J=7.55 Hz, 1H) 7.76 (d, J=8.31 Hz, 1H) 8.02 (d, J=8.31 Hz, 1H) 8.37 (br. s., 1H) 8.51 (m, 2H)

### Example 874

1H NMR (600 MHz, DMSO-d₆) δ ppm 0.60-0.65 (m, 2H) 0.75 (t, J=7.18 Hz, 3H) 0.80-0.85 (m, 2H) 0.87 (m, 2H) 1.21 (m, 2H) 1.30 (q, J=3.78 Hz, 2H) 1.47 (m, 2H) 2.17 (t, J=7.55 Hz, 2H) 2.98-3.09 (m, 1H) 4.41 (d, J=6.04 Hz, 2H) 7.27 (d, J=7.55 Hz, 1H) 7.44 (t, J=7.93 Hz, 1H) 7.67-7.73 (m, 2H) 7.76 (d, J=9.06 Hz, 1H) 8.17 (dd, J=8.69, 1.89 Hz, 1H) 8.28 (t, J=6.04 Hz, 1H) 8.51 (s, 1H) 8.53 (s, 2H) 8.59 (s, 1H)

### Example 892

1H NMR (600 MHz, DMSO-d₆) δ ppm 0.66-0.70 (m, 2H) 0.79-0.89 (m, 6H) 2.63 (ddd, J=6.61, 3.21, 3.02 Hz, 1H) 3.06 (td, J=7.18, 3.78 Hz, 1H) 4.88 (s, 2H) 6.82 (d, J=3.78 Hz, 1H) 7.21 (d, J=3.78 Hz, 1H) 7.26 (d, J=7.55 Hz, 1H) 7.45 (t, J=7.55 Hz, 1H) 7.64-7.71 (m, 2H) 7.75 (d, J=8.31 Hz, 1H) 8.00 (dd, J=8.69, 1.89 Hz, 1H) 8.37 (d, J=3.02 Hz, 1H) 8.50 (d, J=1.51 Hz, 1H) 8.52 (s, 1H)

### Pharmacological Data:

The affinity of the substituted 4-amino-quinazoline compounds corresponding to formula I according to the invention to the mGluR5 receptor can be determined as described above. The pharmacological data for the 4-amino-quinazoline compounds according to the invention are summarized in the following Table 2:

TABLE 2

| Compound | Kᵢ mGluR5 receptor |
|---|---|
| according to | (human) functional |
| Example | [μM] |
| 260 | 0.0008 |
| 273 | 0.0017 |
| 201 | 0.0027 |
| 202 | 0.0033 |
| 258 | 0.0039 |
| 196 | 0.0047 |
| 250 | 0.0050 |
| 245 | 0.0052 |
| 262 | 0.0059 |
| 121 | 0.0075 |
| 269 | 0.0080 |
| 271 | 0.0082 |
| 211 | 0.0084 |
| 270 | 0.0120 |
| 261 | 0.0390 |

| | | IC₅₀ | |
|---|---|---|---|
| | % Inhibition of | mGluR5 Receptor | |
| Compound | mGluR5 Receptor | (pig) | |
| according to | (pig) [³H]-MPEP Bond | [³H]-MPEP Bond | |
| Example | at 10 μM | [μM] | Formalin Test |
| 13 | 36 | | |
| 22 | 32 | | |
| 29 | 46 | | |
| 31 | 32 | | |
| 36 | 42 | | |
| 44 | 37 | | |
| 45 | 48 | | |
| 50 | 30 | | |
| 53 | 38 | | |
| 54 | 47 | | |
| 62 | 43 | | |
| 68 | 30 | | |
| 69 | 38 | | |
| 70 | 34 | | |
| 75 | 32 | | |
| 89 | 52 | | |
| 90 | 44 | | |
| 91 | 54 | | |
| 92 | 74 | | |
| 93 | 38 | | |
| 94 | 75 | | |
| 95 | 61 | | |
| 100 | 36 | | |
| 104 | 48 | | |
| 106 | 39 | | |
| 108 | 31 | | |
| 110 | 43 | | |
| 111 | 46 | | |
| 112 | 33 | | |
| 115 | 50 | | |
| 116 | 47 | | |
| 117 | 68 | | |
| 119 | 52 | | |
| 120 | 48 | | |
| 122 | 37 | | |
| 125 | 30 | | |
| 133 | 50 | | |
| 134 | 58 | | |
| 135 | 32 | | |
| 142 | 51 | | |
| 143 | 71 | | |
| 145 | 53 | | |
| 148 | 40 | | |
| 149 | 32 | | |
| 150 | 40 | | |
| 151 | 66 | | |
| 152 | 45 | | |
| 153 | 63 | | |
| 154 | 37 | | |
| 155 | 67 | | |
| 156 | 90 | | |
| 157 | 90 | | |
| 158 | 81 | | |
| 159 | 85 | | |
| 160 | 70 | | |
| 161 | 96 | | |
| 162 | 84 | | |
| 163 | 89 | | |
| 164 | 86 | | |
| 165 | 88 | | |
| 166 | 86 | | |
| 167 | 98 | | |
| 168 | 90 | | |
| 169 | 91 | | |
| 170 | 78 | | |
| 171 | 100 | | |
| 172 | 99 | | |
| 173 | 30 | | |
| 174 | 97 | | |
| 175 | 85 | | |
| 176 | 76 | | |
| 177 | 97 | | |
| 178 | 84 | | |
| 179 | 89 | | |
| 189 | | 0.21 | |
| 190 | 58 | | |
| 191 | | 0.67 | |
| 192 | 41 | | |
| 193 | | 0.27 | |
| 195 | 40 | | |
| 196 | | 0.09 | |
| 197 | | 0.66 | |
| 198 | | 5.19 | |
| 199 | | 1.25 | |
| 200 | 40 | | |
| 201 | | 0.03 | 46% effect at |
| | | | 2.15 mg/kg i.v. |
| 202 | 55 | | |
| 203 | 39 | | |
| 205 | | 0.33 | |
| 206 | 34 | | |
| 207 | | 7.17 | |
| 208 | | 0.12 | |
| 209 | 63 | | |
| 210 | | 6.07 | |
| 211 | | 0.07 | |
| 212 | | 0.10 | |
| 213 | 32 | | |
| 214 | | 6.92 | |
| 215 | | 3.61 | |
| 216 | | 1.50 | |
| 217 | | 0.51 | |
| 218 | | 2.99 | |
| 219 | 45 | | |
| 220 | | 4.25 | |
| 221 | | 5.36 | |
| 222 | | 4.61 | |
| 223 | 51 | | |
| 225 | | 1.48 | |
| 226 | | 2.64 | |
| 227 | | 2.57 | |
| 228 | | 8.70 | |
| 229 | | 3.50 | |
| 231 | | 5.72 | |
| 232 | | 4.27 | |
| 233 | | 0.82 | |
| 234 | 41 | | |
| 235 | | 7.35 | |
| 237 | | 0.57 | |
| 238 | | 0.50 | |
| 239 | | 4.12 | |
| 240 | 47 | | |
| 241 | 33 | | |
| 242 | | 3.08 | |
| 243 | 39 | | |
| 244 | | 0.22 | |
| 245 | | 0.14 | |
| 246 | | 0.35 | |
| 247 | | 7.34 | |
| 248 | | 3.15 | |
| 249 | | 0.14 | |
| 250 | | 0.12 | |
| 251 | 30 | | |
| 252 | | 0.92 | |
| 253 | | 1.31 | |
| 254 | | 0.60 | |
| 255 | | 1.85 | |
| 256 | | 1.47 | |
| 257 | | 0.74 | |
| 258 | | 0.05 | |
| 259 | | 0.29 | |
| 260 | | 0.03 | 80% effect at |
| | | | 10 mg/kg i.v. |
| 261 | | 0.20 | |
| 262 | | 0.11 | |
| 263 | | 1.70 | |
| 264 | | 1.52 | |
| 265 | | 1.89 | |
| 266 | | 1.27 | |
| 267 | | 6.55 | |
| 268 | | 5.41 | |
| 269 | | 0.28 | |
| 270 | | 0.28 | |
| 271 | | 0.14 | |
| 272 | | 15.32 | |
| 273 | | 0.03 | |
| 274 | | 0.86 | |
| 275 | | 1.38 | |
| 276 | | 1.25 | |
| 277 | | 0.04 | |
| 278 | | 0.35 | |
| 279 | | 9.42 | |
| 281 | | 0.03 | |
| 282 | | 0.05 | |
| 283 | | 0.29 | |
| 286 | | 0.39 | |
| 287 | | 0.06 | |
| 291 | 49 | | |
| 292 | 95 | | |
| 294 | | 8.67 | |
| 295 | 0 | | |
| 298 | | 10.78 | |
| 299 | | 2.01 | |
| 300 | 51 | | |
| 304 | | 3.11 | |
| 306 | 33 | | |
| 307 | 37 | | |
| 310 | 50 | | |
| 312 | 58 | | |
| 315 | | 2.74 | |
| 320 | 72 | | |
| 322 | | 2.98 | |
| 324 | 55 | | |
| 329 | 57 | | |
| 330 | 47 | | |

| | % Inhibition of | IC₅₀ mGluR5 | |
|---|---|---|---|
| Compound | mGluR5 Receptor | Receptor (Pig) | Kᵢ mGluR5 |
| according to | (pig) [³H]-MPEP | [³H]-MPEP Bond | Receptor (human) |
| Example | Bond at 10 μM | [μM] | functional [μM] |
| 366 | 90 | | |
| 367 | 94 | | |
| 368 | 85 | | |
| 369 | 34 | | |
| 370 | 52 | | |
| 374 | 91 | | |
| 375 | 34 | | |
| 377 | 59 | | |
| 380 | 46 | | |
| 381 | 90 | | |
| 382 | 89 | | |
| 383 | 69 | | |
| 385 | 35 | | |
| 389 | 91 | | |
| 390 | | 0.07 | |
| 391 | 74 | | |
| 393 | 95 | | |
| 395 | 44 | | |
| 397 | 87 | | |
| 398 | 63 | | |
| 399 | 39 | | |
| 401 | 36 | | |
| 402 | 34 | | |
| 403 | 40 | | |
| 405 | 77 | | |
| 406 | 95 | | |
| 407 | 92 | | |
| 409 | 32 | | |
| 411 | 34 | | |
| 413 | 89 | | |
| 414 | 93 | | |
| 415 | 53 | | |
| 417 | 60 | | |
| 419 | 76 | | |
| 420 | 94 | | |
| 421 | 81 | | |
| 427 | 95 | | |
| 428 | 96 | | |
| 429 | 92 | | |
| 434 | 62 | | |
| 435 | 87 | | |
| 436 | 98 | | |
| 437 | 90 | | |
| 440 | 73 | | |
| 447 | 85 | | |
| 452 | 71 | | |
| 454 | 34 | | |
| 457 | | 0.21 | |
| 462 | 48 | | |
| 470 | 93 | | |
| 484 | 63 | | |
| 485 | 32 | | |
| 509 | 31 | | |
| 517 | 59 | | |
| 519 | 76 | | |
| 524 | 85 | | |
| 532 | 61 | | |
| 540 | 91 | | |
| 542 | 33 | | |
| 544 | 66 | | |
| 549 | 32 | | |
| 550 | 40 | | |
| 557 | 42 | | |
| 558 | 42 | | |
| 566 | 61 | | |
| 574 | 106 | | |
| 578 | 76 | | |
| 582 | 80 | | |
| 585 | 70 | | |
| 586 | 62 | | |
| 593 | 52 | | |
| 595 | 53 | | |
| 601 | 78 | | |
| 604 | 41 | | |
| 605 | 94 | | |
| 608 | 36 | | |
| 609 | 30 | | |
| 619 | 98 | | |
| 624 | 75 | | |
| 639 | 46 | | |
| 644 | 85 | | |
| 645 | 90 | | |
| 652 | 93 | | |
| 653 | 66 | | |
| 654 | 82 | | |
| 657 | 36 | | |
| 659 | 86 | | |
| 660 | 94 | | |
| 661 | 89 | | |
| 662 | 54 | | |
| 666 | 43 | | |
| 667 | 40 | | |
| 668 | 88 | | |
| 669 | 77 | | |
| 670 | 79 | | |
| 671 | 40 | | |
| 675 | 48 | | |
| 676 | 75 | | |
| 677 | 93 | | |
| 678 | | 0.22 | |
| 683 | 31 | | |
| 684 | 96 | | |
| 685 | 81 | | |
| 686 | 68 | | |
| 687 | 60 | | |
| 689 | 41 | | |
| 691 | 83 | | |
| 692 | 67 | | |
| 693 | 102 | | |
| 694 | 82 | | |
| 697 | 90 | | |
| 698 | 89 | | |
| 699 | 90 | | |
| 700 | 81 | | |
| 701 | 93 | | |
| 702 | 86 | | |
| 703 | 87 | | |
| 705 | 86 | | |
| 706 | 69 | | |
| 707 | 89 | | |
| 708 | | 0.13 | |
| 709 | 36 | | |
| 710 | | 0.11 | |
| 713 | 51 | | |
| 714 | 95 | | |
| 715 | 90 | | |
| 716 | | 0.12 | |
| 718 | 40 | | |
| 719 | 84 | | |
| 721 | 87 | | |
| 722 | 72 | | |
| 723 | 43 | | |
| 724 | 86 | | |
| 726 | 65 | | |
| 727 | 93 | | |
| 728 | 63 | | |
| 730 | 35 | | |
| 731 | 86 | | |
| 732 | 63 | | |
| 733 | 39 | | |
| 734 | 91 | | |
| 735 | 72 | | |
| 736 | 91 | | |
| 737 | 74 | | |
| 738 | 90 | | |
| 739 | 47 | | |
| 740 | 85 | | |
| 741 | 37 | | |
| 742 | 69 | | |
| 744 | 90 | | |
| 745 | 52 | | |
| 746 | 93 | | |
| 747 | 52 | | |
| 748 | 66 | | |
| 749 | 65 | | |
| 750 | 42 | | |
| 751 | 53 | | |
| 752 | 62 | | |
| 754 | 95 | | |
| 755 | 78 | | |
| 756 | 69 | | |
| 759 | 83 | | |
| 760 | 93 | | |
| 761 | 45 | | |
| 762 | 91 | | |
| 763 | 86 | | |
| 764 | 87 | | |
| 768 | 88 | | |
| 769 | 83 | | |
| 770 | 84 | | |
| 771 | 80 | | |
| 773 | 74 | | |
| 775 | 76 | | |
| 776 | 55 | | |
| 777 | | 0.05 | |
| 778 | 84 | | |
| 781 | 67 | | |
| 782 | 50 | | |
| 783 | 93 | | |
| 784 | 56 | | |
| 785 | 100 | | |
| 786 | 95 | | |
| 788 | 77 | | |
| 789 | 84 | | |
| 791 | 89 | | |
| 792 | 88 | | |
| 793 | 94 | | |
| 794 | 93 | | |
| 795 | 73 | | |
| 796 | 78 | | |
| 797 | 85 | | |
| 799 | 92 | | |
| 800 | 91 | | |
| 801 | 88 | | |
| 802 | 97 | | |
| 803 | 93 | | |
| 804 | 83 | | |
| 805 | 92 | | |
| 806 | 89 | | |
| 807 | 90 | | |
| 808 | 81 | | |
| 809 | 48 | | |
| 810 | 45 | | |
| 811 | 71 | | |
| 812 | 73 | | |
| 813 | 73 | | |
| 814 | 89 | | |
| 815 | 87 | | |
| 817 | 71 | | |
| 818 | 34 | | |
| 819 | 88 | | |
| 820 | 48 | | |
| 821 | 84 | | |
| 822 | 86 | | |
| 823 | 62 | | |
| 824 | 71 | | |
| 825 | 97 | | |
| 826 | 98 | | |
| 827 | 87 | | |
| 828 | 74 | | |
| 829 | 45 | | |
| 830 | 90 | | |
| 831 | 84 | | |
| 832 | 96 | | |
| 833 | 43 | | |
| 834 | 42 | | |
| 835 | 58 | | |
| 837 | 86 | | |
| 838 | 92 | | |
| 840 | 74 | | |
| 841 | 36 | | |
| 843 | 71 | | |
| 844 | 94 | | |
| 846 | 82 | | |
| 847 | 43 | | |
| 848 | 53 | | |
| 849 | 86 | | |
| 850 | 83 | | |
| 852 | 49 | | |
| 853 | 67 | | |
| 854 | 75 | | |
| 855 | 83 | | |
| 856 | 89 | | |
| 857 | 36 | | |
| 858 | 85 | | |
| 859 | 92 | | |
| 860 | 63 | | |
| 861 | 55 | | |
| 866 | | 0.09 | |
| 867 | 67 | | |
| 868 | | | |
| 869 | 65 | | |
| 870 | | 0.01 | |
| 871 | | | |
| 872 | | 0.05 | |
| 873 | | 0.08 | |
| 874 | | | |
| 875 | | 0.04 | 0.0511 |
| 876 | 47 | | |
| 877 | | 9.57 | |
| 878 | 56 | | |
| 879 | | 1.66 | |
| 880 | | 4.30 | |
| 881 | | 0.02 | |
| 882 | | 0.07 | 0.0027 |
| 883 | | 0.02 | |
| 884 | | 0.13 | |
| 885 | | 0.40 | |
| 886 | | 1.98 | |
| 887 | | 3.10 | |
| 888 | | 2.65 | |
| 889 | | 0.50 | |
| 890 | | 4.62 | |
| 891 | | 5.60 | |
| 892 | | 0.08 | |
| 893 | | 3.21 | |
| 894 | | 1.00 | |
| 895 | 72 | | |
| 896 | 78 | | |
| 897 | | 0.02 | |
| 898 | | 0.09 | |
| 899 | | 0.16 | |
| 900 | 81 | | |
| 901 | 40 | | |
| 902 | 92 | | |
| 903 | 87 | | |
| 904 | | 0.22 | |

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A substituted 4-amino-quinazoline compound corresponding to formula I 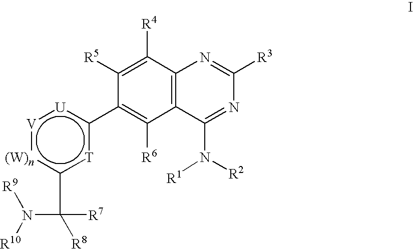
wherein
T represents CR¹¹, N, S or O;
U represents CR¹², N, S or O;
V represents CR¹³, N, S or O;
W represents CR¹⁴ or N;
n is 0 or 1;
R¹ and R² each independently represent H; —C(═O)—OH; —C(═O)—H; —C(═O)—R¹⁵; —C(═O)—O—R¹⁶; —C(═O)—NH₂; —C(═O)—NH—R¹⁷; —C(═S)—NH—R¹⁷; C(═O)—NR¹⁸R¹⁹; —C(═S)—NR¹⁸R¹⁹; —S(═O)—R²⁰; —S(═O)₂—R²¹; unsubstituted or mono- or poly-substituted alkyl, alkenyl or alkinyl; unsubstituted or mono- or poly-substituted heteroalkyl, heteroalkenyl or heteroalkinyl; unsubstituted or mono- or poly-substituted cycloalkyl or cycloalkenyl; unsubstituted or mono- or poly-substituted heterocycloalkyl or heterocycloalkenyl; unsubstituted or mono- or poly-substituted -(alkylene)-cyloalkyl, -(alkenylene)-cycloalkyl, -(alkinylene)-cycloalkyl, -(alkylene)-cycloalkenyl, -(alkenylene)-cycloalkenyl or -(alkinylene)-cycloalkenyl; unsubstituted or mono- or poly-substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or mono- or poly-substituted -(alkylene)-heterocycloalkyl, -(alkenylene)-heterocycloalkyl, -(alkinylene)-heterocycloalkyl, -(alkylene)-heterocycloalkenyl, -(alkenylene)-heterocycloalkenyl or -(alkinylene)-heterocycloalkenyl; unsubstituted or mono- or poly-substituted -(heteroalkylene)-heterocycloalkyl, (heteroalkenylene)-heterocycloalkyl; -(heteroalkylene)-heterocycloalkenyl or -(heteroalkenylene)-heterocycloalkenyl; aryl that may be unsubstituted or substituted with optionally 1, 2, 3, 4 or 5 substituents independently selected from the group consisting of F, Cl, Br, I, —CN, —NO₂, —OH, —SH, —NH₂, —C(═O)—OH, —C₁₋₅-alkyl, —(CH₂)—O—C₁₋₅-alkyl, —C₂₋₅-alkenyl, —C₂₋₅-alkinyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, —S(═O)₂—C₁₋₅-alkyl, —S(═O)—C₁₋₅-alkyl, —NH—C₁₋₅-alkyl, N(C₁₋₅-alkyl)₂, —C(═O)—O—C₁₋₅-alkyl, —C(═O)—H, —C(═O)—C₁₋₅-alkyl, —CH₂—O—C(═O)-phenyl, —O—C(═O)-phenyl, —NH—S(═O)₂—C₁₋₅-alkyl, —NH—C(═O)—C₁₋₅-alkyl, —C(═O)—NH₂, —C(═O)—NH—C₁₋₅-alkyl, —C(═O)—N(C₁₋₅-alkyl)₂, cyclopropyl, cyclobutyl, cyclopentyl, cyclohexyl, piperazinyl, pyrrolidinyl, piperidinyl, pyrazolyl, phenyl, furyl, thiazolyl, thiadiazolyl, thiophenyl, benzyl and phenethyl, wherein the cyclic substituents or the cyclic residues of these substituents can themselves be substituted with optionally 1, 2, 3, 4 or 5 substituents independently selected from the group consisting of F, Cl, Br, I, —CN, —NO₂, —OH, —SH, —NH₂, —C(═O)—OH, —C₁₋₅-alkyl, —(CH₂)—O—C₁₋₅-alkyl, —C₂₋₅-alkenyl, —C₂₋₅-alkinyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂, —S—CH₂F; unsubstituted or mono- or poly-substituted heteroaryl; unsubstituted or mono- or poly-substituted -(alkylene)-aryl, -(alkenylene)-aryl, (alkinylene)-aryl, (heteroalkylene)-aryl or -(heteroalkenylene)-aryl; or unsubstituted or mono- or poly-substituted -(alkylene)-heteroaryl, -(alkenylene)-heteroaryl, -(alkinylene)-heteroaryl, -(heteroalkylene)-heteroaryl or -(heteroalkenylene)-heteroaryl; or
R¹ and R² together with the nitrogen atom to which they are bound represent unsubstituted or mono- or poly-substituted heterocycloalkyl or heterocycloalkenyl;
R³ represents H; F; Cl; Br; I; —NO₂; —CN, —NH₂, —OH; —SH; —C(═O)—OH; —C(═O)—H; —C(═O)—R¹⁵; —C(═O)—O—R¹⁶; —C(═O)—NH₂; —C(═O)—NH—R¹⁷; —C(═S)—NH—R¹⁷; —C(═O)—NR¹⁸R¹⁹; —C(═S)—NR¹⁸R¹⁹; —S(═O)—R²⁰; —S(═O)₂—R²¹; —NH—R²²; —NR²³R²⁴; —O—R²⁵; —S—R²⁶; —O—C(═O)—R²⁷; —NH—C(═O)—H; —NH—C(═O)—R²⁸; —NR²⁹—C(═O)—R³⁰; —NH—C(═O)—NH—R³¹; —NH—C(═S)—NH—R³²; —NH—S(═O)₂—R³³; —NR³⁴—S(═O)₂—R³⁵; unsubstituted or mono- or poly-substituted alkyl, alkenyl or alkinyl; unsubstituted or mono- or poly-substituted heteroalkyl, heteroalkenyl or heteroalkinyl; unsubstituted or mono- or poly-substituted cycloalkyl or cycloalkenyl; unsubstituted or mono- or poly-substituted heterocycloalkyl or heterocycloalkenyl; unsubstituted or mono- or poly-substituted aryl; unsubstituted or mono- or poly-substituted heteroaryl; unsubstituted or mono- or poly-substituted -(alkylene)-aryl, -(alkenylene)-aryl, (alkinylene)-aryl, (heteroalkylene)-aryl or -(heteroalkenylene)-aryl; or unsubstituted or mono- or poly-substituted -(alkylene)-heteroaryl, -(alkenylene)-heteroaryl, -(alkinylene)-heteroaryl, -(heteroalkylene)-heteroaryl or -(heteroalkenylene)-heteroaryl;
R⁴, R⁵ and R⁶ each independently represent H; F; Cl; Br; I; —NO₂; —CN, —NH₂, —OH; —SH; —C(═O)—OH; —C(═O)—H; —C(═O)—R¹⁵; —C(═O)—O—R¹⁶; —C(═O)—NH₂; —C(═O)—NH—R¹⁷; —C(═S)—NH—R¹⁷; —C(═O)—NR¹⁸R¹⁹; —C(═S)—NR¹⁸R¹⁹; —S(═O)—R²⁰; —S(═O)₂—R²¹; —NH—R²²; —NR²³R²⁴; —O—R²⁵; —S—R²⁶; —O—C(═O)—R²⁷; —NH—C(═O)—H; —NH—C(═O)—R²⁸; —NR²⁹—C(═O)—R³⁰; —NH—C(═O)—NH—R³¹; —NH—C(═S)—NH—R³²; —NH—S(═O)₂—R³³; —NR³⁴—S(═O)₂—R³⁵; unsubstituted or mono- or poly-substituted alkyl, alkenyl or alkinyl; unsubstituted or mono- or poly-substituted heteroalkyl, heteroalkenyl or heteroalkinyl; unsubstituted or mono- or poly-substituted cycloalkyl or cycloalkenyl; unsubstituted or mono- or poly-substituted heterocycloalkyl or heterocycloalkenyl; unsubstituted or mono- or poly-substituted aryl; unsubstituted or mono- or poly-substituted heteroaryl; unsubstituted or mono- or poly-substituted -(alkylene)-aryl, -(alkenylene)-aryl, (alkinylene)-aryl, (heteroalkylene)-aryl or -(heteroalkenylene)-aryl; or unsubstituted or mono- or poly-substituted -(alkylene)-heteroaryl, -(alkenylene)-heteroaryl, -(alkinylene)-heteroaryl, -(heteroalkylene)-heteroaryl or -(heteroalkenylene)-heteroaryl;
R⁷ and R⁸ each independently represent H; F; Cl; Br; I; —NO₂; —CN, —NH₂, —OH; —SH; —C(═O)—OH; —C(═O)—H; —C(═O)—R¹⁵; —C(═O)—O—R¹⁶; —C(═O)—NH₂; —C(═O)—NH—R¹⁷; —C(═S)—NH—R¹⁷; —C(═O)—NR¹⁸R¹⁹; —C(═S)—NR¹⁸R¹⁹; —S(═O)—R²⁰; —S(═O)₂—R²¹; —NH—R²²; —NR²³R²⁴; —O—R²⁵; —S—R²⁶; —O—C(═O)—R²⁷; —NH—C(═O)—H; —NH—C(═O)—R²⁸; —NR²⁹—C(═O)—R³⁰; —NH—C(═O)—NH—R³¹; —NH—C(═S)—NH—R³²; —NH—S(═O)₂—R³³; —NR³⁴—S(═O)₂—R³⁵; unsubstituted or mono- or poly-substituted alkyl, alkenyl or alkinyl; unsubstituted or mono- or poly-substituted heteroalkyl, heteroalkenyl or heteroalkinyl; unsubstituted or mono- or poly-substituted cycloalkyl or cycloalkenyl; unsubstituted or mono- or poly-substituted heterocycloalkyl or heterocycloalkenyl; unsubstituted or mono- or poly-substituted aryl; unsubstituted or mono- or poly-substituted heteroaryl; unsubstituted or mono- or poly-substituted -(alkylene)-aryl, -(alkenylene)-aryl, (alkinylene)-aryl, (heteroalkylene)-aryl or -(heteroalkenylene)-aryl; or unsubstituted or mono- or poly-substituted -(alkylene)-heteroaryl, -(alkenylene)-heteroaryl, -(alkinylene)-heteroaryl, -(heteroalkylene)-heteroaryl or -(heteroalkenylene)-heteroaryl;
R⁹ represents H, —C(═O)—OH; —C(═O)—H; —C(═O)—R¹⁵; —C(═O)—O—R¹⁶; —C(═O)—NH₂; —C(═O)—NH—R¹⁷; —C(═S)—NH—R¹⁷; —C(═O)—NR¹⁸R¹⁹; —C(═S)—NR¹⁸R¹⁹; —S(═O)—R²⁰; —S(═O)₂—R²¹; unsubstituted or mono- or poly-substituted alkyl, alkenyl or alkinyl; unsubstituted or mono- or poly-substituted heteroalkyl, heteroalkenyl or heteroalkinyl; unsubstituted or mono- or poly-substituted cycloalkyl or cycloalkenyl; unsubstituted or mono- or poly-substituted heterocycloalkyl or heterocycloalkenyl; unsubstituted or mono- or poly-substituted -(alkylene)-cyloalkyl, -(alkenylene)-cycloalkyl, -(alkinylene)-cycloalkyl, -(alkylene)-cycloalkenyl, -(alkenylene)-cycloalkenyl or -(alkinylene)-cycloalkenyl; unsubstituted or mono- or poly-substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or mono- or poly-substituted -(alkylene)-heterocycloalkyl, -(alkenylene)-heterocycloalkyl, -(alkinylene)-heterocycloalkyl, -(alkylene)-heterocycloalkenyl, -(alkenylene)-heterocycloalkenyl or -(alkinylene)-heterocycloalkenyl; unsubstituted or mono- or poly-substituted -(heteroalkylene)-heterocycloalkyl, (heteroalkenylene)-heterocycloalkyl-(heteroalkylene)-heterocycloalkenyl or -(heteroalkenylene)-heterocycloalkenyl; unsubstituted or mono- or poly-substituted aryl; unsubstituted or mono- or poly-substituted heteroaryl; unsubstituted or mono- or poly-substituted -(alkylene)-aryl, -(alkenylene)-aryl, (alkinylene)-aryl, (heteroalkylene)-aryl or -(heteroalkenylene)-aryl; or unsubstituted or mono- or poly-substituted -(alkylene)-heteroaryl, -(alkenylene)-heteroaryl, -(alkinylene)-heteroaryl, -(heteroalkylene)-heteroaryl or -(heteroalkenylene)-heteroaryl;
R¹⁰ represents —C(═O)—OH; —C(═O)—H; —C(═O)—R¹⁵; —C(═O)—O—R¹⁶; —C(═O)—NH₂; —C(═O)—NH—R¹⁷; —C(═S)—NH—R¹⁷; —C(═O)—NR¹⁸R¹⁹; —C(═S)—NR¹⁸R¹⁹; —S(═O)—R²⁰; —S(═O)₂—R²¹; alkyl, alkenyl or alkinyl, which is respectively substituted with 1, 2, 3, 4 or 5 substituents independently selected from the group consisting of F, Cl, Br, I, —NO₂, —CN, —SH, —NH₂, —N(C₁₋₅)-alkyl)₂, —N(C₁₋₅-alkyl)(phenyl), —N(C₁₋₅-alkyl)(CH₂-phenyl), —N(C₁₋₅-alkyl)(CH₂—CH₂-phenyl), NH—C(═O)—O—C₁₋₅-alkyl, —C(═O)—H, —C(═O)—C₁₋₅-alkyl, —C(═O)-phenyl, —C(═S)—C₁₋₅-alkyl, —C(═S)-phenyl, —C(═O)—OH, —C(═O)—O—C₁₋₅-alkyl, —C(═O)—O-phenyl, C(═O)—NH₂, —C(═O)—NH—C₁₋₅-alkyl, —C(═O)—N(C₁₋₅-alkyl)₂, —S(═O)—C₁₋₅-alkyl, —S(═O)-phenyl, —S(═O)₂-phenyl, —S(═O)₂—NH₂ and —SO₃H, wherein the phenyl residues can be substituted with 1, 2, 3, 4 or 5 substituents independently selected from the group consisting of F, Cl, Br, I, —CN, CF₃, —OH, —NH₂, O—CF₃, —SH, —O—CH₃, —O—C₂H₅, —O—C₃H₇, methyl, ethyl, n-propyl, isopropyl, n-butyl, 2-butyl, isobutyl and tert-butyl; unsubstituted or mono- or poly-substituted heteroalkenyl or heteroalkinyl; unsubstituted or mono- or poly-substituted cycloalkyl or cycloalkenyl; unsubstituted or mono- or poly-substituted heterocycloalkyl or heterocycloalkenyl; unsubstituted or mono- or poly-substituted -(alkylene)-cyloalkyl, -(alkenylene)-cycloalkyl, -(alkinylene)-cycloalkyl, -(alkylene)-cycloalkenyl, -(alkenylene)-cycloalkenyl or -(alkinylene)-cycloalkenyl; unsubstituted or mono- or poly-substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or mono- or poly-substituted -(alkylene)-heterocycloalkyl, -(alkenylene)-heterocycloalkyl, -(alkinylene)-heterocycloalkyl, -(alkylene)-heterocycloalkenyl, -(alkenylene)-heterocycloalkenyl or -(alkinylene)-heterocycloalkenyl; unsubstituted or mono- or poly-substituted -(heteroalkylene)-heterocycloalkyl, (heteroalkenylene)-heterocycloalkyl-(heteroalkylene)-heterocycloalkenyl or -(heteroalkenylene)-heterocycloalkenyl; unsubstituted or mono- or poly-substituted aryl; unsubstituted or mono- or poly-substituted heteroaryl; unsubstituted or mono- or poly-substituted -(alkylene)-aryl, -(alkenylene)-aryl, (alkinylene)-aryl, (heteroalkylene)-aryl or -(heteroalkenylene)-aryl; or unsubstituted or mono- or poly-substituted -(alkylene)-heteroaryl, -(alkenylene)-heteroaryl, -(alkinylene)-heteroaryl, -(heteroalkylene)-heteroaryl or -(heteroalkenylene)-heteroaryl; or
R⁹ and R¹⁰ together with the nitrogen atom to which they are bound form a residue selected from the group consisting of imidazolidinyl, azepanyl, pyrrolyl, diazepanyl, isothioazolidinyl, (2,3)-dihydro-1H-isoindolyl, indolinyl, octahydro-1H-isoindolyl, (2,3)-dihydropyrrolyl, (2,5)-dihydropyrrolyl, 8-azaspiro[4.5]decyl, 8-azaspiro[4.5]decane-7,9-dionyl, isoindoline-1,3-dionyl, pyrrolidine-2,5-dionyl, morpholine-3,5-dionyl, 1H-pyrrole-2,5-dionyl, piperidine-2,6-dionyl, hexahydro-2H-isoindoline-1,3-dionyl, pyrrolidine-2-onyl and 1,1-dioxo-1λ⁶-isothiazolidinyl, each of which may be unsubstituted or optionally substituted with 1, 2, 3, 4 or 5 substituents independently selected from the group consisting of F, Cl, Br, I, —CN, CF₃, —OH, —NH₂, O—CF₃, —SH, —O—C₁₋₅-alkyl, —O-phenyl, —O—CH₂-phenyl, —(CH₂)—O—C₁₋₅-alkyl, —S—C₁₋₅-alkyl, —S-phenyl, —S—CH₂-phenyl, —C₁₋₅-alkyl, —C₂₋₅-alkenyl, —C₂₋₅-alkinyl, —C≡C—Si(CH₃)₃, —C≡C—Si(C₂H₅)₃, —C(═O)—O—C₁₋₅-alkyl, —C(═O)—CF₃, —S(═O)₂—C₁₋₅-alkyl, —S(—O)—C₁₋₅-alkyl, —S(═O)₂-phenyl, thioxo (═S), —N(C₁₋₅-alkyl)₂, —N(H)(C₁₋₅-alkyl), —NO₂, —S—CF₃, —C(═O)—OH, —NH—S(═O)₂—C₁₋₅-alkyl, —NH—C(═O)—C₁₋₅-alkyl, —NH—C(═O)—CF₃, —C(═O)—H, —C(═O)—C₁₋₅-alkyl, —C(═O)—NH₂, —C(═O)—N(C₁₋₅-alkyl)₂, —C(═O)—N(H)(C₁₋₅-alkyl) and phenyl, wherein the phenyl residues can be respectively unsubstituted or substituted with 1, 2, 3, 4 or 5 substituents independently selected from the group consisting of F, Cl, Br, I, —CN, —CF₃, —OH, —NH₂, —O—CF₃, —SH, —O—C₁₋₅-alkyl, —O-phenyl, —O—CH₂-phenyl, —(CH₂)—O—C₁₋₅-alkyl, —S—C₁₋₅-alkyl, —S-phenyl, —S—CH₂— phenyl, —C₁₋₅-alkyl, —C₂₋₅-alkenyl, —C₂₋₅-alkinyl, —C≡C—Si(CH₃)₃, —C≡C—Si(C₂H₅)₃, —C(═O)—O—C₁₋₅-alkyl and —C(═O)—CF₃, wherein the abovementioned phenyl residues can be substituted with 1, 2, 3, 4 or 5 substituents independently selected from the group consisting of F, Cl, Br, I, —CN, —CF₃, —OH, —NH₂, —O—CF₃, —SH, —O—CH₃, —O—C₂H₅, —O—C₃H₇, methyl, ethyl, n-propyl, isopropyl, n-butyl, 2-butyl, isobutyl and tert-butyl; and
if R¹ represents a residue selected from the group consisting of methyl, ethyl, n-propyl, isopropyl, n-butyl, isobutyl, 2-butyl, tert-butyl and cyclopropyl, then R¹⁰ can additionally represent H, unsubstituted alkyl or unsubstituted or at least substituted heteroalkyl;
R¹¹, R¹², R¹³ and R¹⁴ each independently represent H; F; Cl; Br; I; —NO₂; —CN, —NH₂, —OH; —SH; —C(═O)—OH; —C(═O)—H; —C(═O)—R¹⁵; —C(═O)—O—R¹⁶; —C(═O)—NH₂; —C(═O)—NH—R¹⁷; —C(═S)—NH—R¹⁷; —C(═O)—NR¹⁸R¹⁹; —C(═S)—NR¹⁸R¹⁹; —S(═O)—R²⁰; —S(═O)₂—R²¹; —NH—R²²; —NR²³R²⁴; —O—R²⁵; —S—R²⁶; —O—C(═O)—R²⁷; —NH—C(═O)—H; —NH—C(═O)—R²⁸; —NR²⁹—C(═O)—R³⁰; —NH—C(═O)—NH—R³¹; —NH—C(═S)—NH—R³²; —NH—S(═O)₂—R³³; —NR³⁴—S(═O)₂—R³⁵; unsubstituted or mono- or poly-substituted alkyl, alkenyl or alkinyl; unsubstituted or mono- or poly-substituted heteroalkyl, heteroalkenyl or heteroalkinyl; unsubstituted or mono- or poly-substituted cycloalkyl or cycloalkenyl; unsubstituted or mono- or poly-substituted heterocycloalkyl or heterocycloalkenyl; unsubstituted or mono- or poly-substituted aryl; unsubstituted or mono- or poly-substituted heteroaryl; unsubstituted or mono- or poly-substituted -(alkylene)-aryl, -(alkenylene)-aryl, (alkinylene)-aryl, (heteroalkylene)-aryl or -(heteroalkenylene)-aryl; or unsubstituted or mono- or poly-substituted -(alkylene)-heteroaryl, -(alkenylene)-heteroaryl, -(alkinylene)-heteroaryl, -(heteroalkylene)-heteroaryl or -(heteroalkenylene)-heteroaryl;
R¹⁵ represents —C(═O)—O—R¹⁶; unsubstituted or mono- or poly-substituted alkyl, alkenyl or alkinyl; unsubstituted or mono- or poly-substituted heteroalkyl, heteroalkenyl or heteroalkinyl; unsubstituted or mono- or poly-substituted cycloalkyl or cycloalkenyl; unsubstituted or mono- or poly-substituted heterocycloalkyl or heterocycloalkenyl; unsubstituted or mono- or poly-substituted -(alkylene)-cyloalkyl, -(alkenylene)-cycloalkyl, or -(alkinylene)-cycloalkyl, -(alkylene)-cycloalkenyl, -(alkenylene)-cycloalkenyl or -(alkinylene)-cycloalkenyl; unsubstituted or mono- or poly-substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or mono- or poly-substituted -(alkylene)-heterocycloalkyl, -(alkenylene)-heterocycloalkyl, -(alkinylene)-heterocycloalkyl, -(alkylene)-heterocycloalkenyl, -(alkenylene)-heterocycloalkenyl or -(alkinylene)-heterocycloalkenyl; unsubstituted or mono- or poly-substituted -(heteroalkylene)-heterocycloalkyl, (heteroalkenylene)-heterocycloalkyl; -(heteroalkylene)-heterocycloalkenyl or -(heteroalkenylene)-heterocycloalkenyl; unsubstituted or mono- or poly-substituted aryl; unsubstituted or mono- or poly-substituted heteroaryl; unsubstituted or mono- or poly-substituted -(alkylene)-aryl, -(alkenylene)-aryl, (alkinylene)-aryl, (heteroalkylene)-aryl or -(heteroalkenylene)-aryl; or unsubstituted or mono- or poly-substituted -(alkylene)-heteroaryl, -(alkenylene)-heteroaryl, -(alkinylene)-heteroaryl, -(heteroalkylene)-heteroaryl or -(heteroalkenylene)-heteroaryl; and
R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², R³³, R³⁴ and R³⁵ each independently represent unsubstituted or mono- or poly-substituted alkyl, alkenyl or alkinyl; unsubstituted or mono- or poly-substituted heteroalkyl, heteroalkenyl or heteroalkinyl; unsubstituted or mono- or poly-substituted cycloalkyl or cycloalkenyl; unsubstituted or mono- or poly-substituted heterocycloalkyl or heterocycloalkenyl; unsubstituted or mono- or poly-substituted -(alkylene)-cyloalkyl, -(alkenylene)-cycloalkyl, or -(alkinylene)-cycloalkyl, -(alkylene)-cycloalkenyl, -(alkenylene)-cycloalkenyl or -(alkinylene)-cycloalkenyl; unsubstituted or mono- or poly-substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or mono- or poly-substituted -(alkylene)-heterocycloalkyl, -(alkenylene)-heterocycloalkyl, -(alkinylene)-heterocycloalkyl, -(alkylene)-heterocycloalkenyl, -(alkenylene)-heterocycloalkenyl or -(alkinylene)-heterocycloalkenyl; unsubstituted or mono- or poly-substituted -(heteroalkylene)-heterocycloalkyl, (heteroalkenylene)-heterocycloalkyl; -(heteroalkylene)-heterocycloalkenyl or -(heteroalkenylene)-heterocycloalkenyl; unsubstituted or mono- or poly-substituted aryl; unsubstituted or mono- or poly-substituted heteroaryl; unsubstituted or mono- or poly-substituted -(alkylene)-aryl, -(alkenylene)-aryl, (alkinylene)-aryl, (heteroalkylene)-aryl or -(heteroalkenylene)-aryl; or unsubstituted or mono- or poly-substituted -(alkylene)-heteroaryl, -(alkenylene)-heteroaryl, -(alkinylene)-heteroaryl, -(heteroalkylene)-heteroaryl or -(heteroalkenylene)-heteroaryl;
or a salt thereof.

2. A compound as claimed in claim 1, wherein said compound is in the form of an isolated stereoisomer.

3. A compound as claimed in claim 1, wherein said compound is in the form of a mixture of stereoisomers in any mixing ratio.

4. A compound as claimed in claim 3, wherein said compound is in the form of a racemic mixture.

5. A compounds as claimed in claim 1, wherein
T represents CR¹¹, N, S or O;
U represents CR¹², N, S or O;
V represents CR¹³, N, S or O;
W represents CR¹⁴ or N;
n represents 0 or 1;
R¹ and R² each independently represent H; —C(═O)—OH; —C(═O)—H; —C(═O)—R¹⁵; —C(═O)—O—R¹⁶; —C(═O)—NH₂; —C(═O)—NH—R¹⁷; —C(═O)—NR¹⁸R¹⁹; —C(═S)—NR¹⁸R¹⁹; —S(═O)—R²⁰; —S(═O)₂—R²¹; unsubstituted or mono- or poly-substituted alkyl, alkenyl or alkinyl; unsubstituted or mono- or poly-substituted heteroalkyl, heteroalkenyl or heteroalkinyl; unsubstituted or mono- or poly-substituted cycloalkyl or cycloalkenyl; unsubstituted or mono- or poly-substituted heterocycloalkyl or heterocycloalkenyl; unsubstituted or mono- or poly-substituted -(alkylene)-cyloalkyl, -(alkenylene)-cycloalkyl, -(alkinylene)-cycloalkyl, -(alkylene)-cycloalkenyl, -(alkenylene)-cycloalkenyl or -(alkinylene)-cycloalkenyl; unsubstituted or mono- or poly-substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or mono- or poly-substituted -(alkylene)-heterocycloalkyl, -(alkenylene)-heterocycloalkyl, -(alkinylene)-heterocycloalkyl, -(alkylene)-heterocycloalkenyl, -(alkenylene)-heterocycloalkenyl or -(alkinylene)-heterocyclo-alkenyl; unsubstituted or mono- or poly-substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or mono- or poly-substituted -(heteroalkylene)-heterocycloalkyl, -(heteroalkenylene)-heterocycloalkyl or -(heteroalkylene)-heterocycloalkenyl or -(heteroalkenylene)-heterocycloalkenyl; unsubstituted or mono- or poly-substituted aryl; unsubstituted or mono- or poly-substituted heteroaryl; unsubstituted or mono- or poly-substituted -(alkylene)-aryl, -(alkenylene)-aryl, (alkinylene)-aryl, (heteroalkylene)-aryl or -(heteroalkenylene)-aryl; or unsubstituted or mono- or poly-substituted -(alkylene)-heteroaryl, -(alkenylene)-heteroaryl, -(alkinylene)-heteroaryl, -(heteroalkylene)-heteroaryl or -(heteroalkenylene)-heteroaryl; or
R¹ and R² together with the nitrogen atom to which they are bound represent unsubstituted or mono- or poly-substituted heterocycloalkyl or heterocycloalkenyl;
R³ represents H; F; Cl; Br; I; —NO₂; —CN, —NH₂, —OH; —SH; —C(═O)—OH; —C(═O)—H; —C(═O)—R¹⁵; —C(═O)—O—R¹⁶; —C(═O)—NH₂; —C(═O)—NH—R¹⁷; —C(═S)—NH—R¹⁷; —C(═O)—NR¹⁸R¹⁹; —C(—S)—NR¹⁸R¹⁹; —S(═O)—R²⁰; —S(═O)₂—R²¹; —NH—R²²; —NR²³R²⁴; —O—R²⁵; —S—R²⁶; —O—C(═O)—R²⁷; —NH—C(═O)—H; —NH—C(═O)—R²⁸; —NR²⁹—C(═O)—R³⁰; —NH—C(═O)—NH—R³¹; —NH—C(═S)—NH—R³²; —NH—S(═O)₂—R³³; —NR³⁴—S(═O)₂—R³⁵; unsubstituted or mono- or poly-substituted alkyl, alkenyl or alkinyl; unsubstituted or mono- or poly-substituted heteroalkyl, heteroalkenyl or heteroalkinyl; unsubstituted or mono- or poly-substituted cycloalkyl or cycloalkenyl; unsubstituted or mono- or poly-substituted heterocycloalkyl or heterocycloalkenyl; unsubstituted or mono- or poly-substituted aryl; unsubstituted or mono- or poly-substituted heteroaryl; unsubstituted or mono- or poly-substituted -(alkylene)-aryl, -(alkenylene)-aryl, (alkinylene)-aryl, (heteroalkylene)-aryl or -(hetero-alkenylene)-aryl; or unsubstituted or mono- or poly-substituted -(alkylene)-heteroaryl, -(alkenylene)-heteroaryl, -(alkinylene)-heteroaryl, -(heteroalkylene)-heteroaryl or -(heteroalkenylene)-heteroaryl;
R⁴, R⁵ and R⁶ each independently represent H; F; Cl; Br; I; —NO₂; —CN, —NH₂, —OH; —SH; —C(═O)—OH; —C(═O)—H; —C(═O)—R¹⁵; —C(═O)—O—R¹⁶; —C(═O)—NH₂; —C(═O)—NH—R¹⁷; —C(═S)—NH—R¹⁷; —C(═O)—NR¹⁸R¹⁹; —C(═S)—NR¹⁸R¹⁹; —S(═O)—R²⁰; —S(═O)₂—R²¹; —NH—R²²; —NR²³R²⁴; —O—R²⁵; —S—R²⁶; —O—C(═O)—R²⁷; —NH—C(═O)—H; —NH—C(═O)—R²⁸; —NR²⁹—C(═O)—R³⁰; —NH—C(═O)—NH—R³¹; —NH—C(═S)—NH—R³²; —NH—S(═O)₂—R³³; —NR³⁴—S(═O)₂—R³⁵; unsubstituted or mono- or poly-substituted alkyl, alkenyl or alkinyl; unsubstituted or mono- or poly-substituted heteroalkyl, heteroalkenyl or heteroalkinyl; unsubstituted or mono- or poly-substituted cycloalkyl or cycloalkenyl; unsubstituted or mono- or poly-substituted heterocycloalkyl or heterocycloalkenyl; unsubstituted or mono- or poly-substituted aryl; unsubstituted or mono- or poly-substituted heteroaryl; unsubstituted or mono- or poly-substituted -(alkylene)-aryl, -(alkenylene)-aryl, (alkinylene)-aryl, (heteroalkylene)-aryl or -(heteroalkenylene)-aryl; or unsubstituted or mono- or poly-substituted -(alkylene)-heteroaryl, -(alkenylene)-heteroaryl, -(alkinylene)-heteroaryl, -(heteroalkylene)-heteroaryl or -(heteroalkenylene)-heteroaryl;
R⁷ and R⁸ each independently represent H; F; Cl; Br; I; —NO₂; —CN, —NH₂, —OH; —SH; —C(═O)—OH; —C(═O)—H; —C(═O)—R¹⁵; —C(═O)—O—R¹⁶; —C(═O)—NH₂; —C(═O)—NH—R¹⁷; —C(═S)—NH—R¹⁷; —C(═O)—NR¹⁸R¹⁹; —C(═S)—NR¹⁸R¹⁹; —S(═O)—R²⁰; —S(═O)₂—R²¹; —NH—R²²; NR²³R²⁴; —O—R²⁵; —S—R²⁶; —O—C(═O)—R²⁷; —NH—C(═O)—H; —NH—C(═O)—R²⁸; —NR²⁹—C(═O)—R³⁰; —NH—C(═O)—NH—R³¹; —NH—C(═S)—NH—R³²; —NH—S(═O)₂—R³³; —NR³⁴—S(═O)₂—R³⁵; unsubstituted or mono- or poly-substituted alkyl, alkenyl or alkinyl; unsubstituted or mono- or poly-substituted heteroalkyl, heteroalkenyl or heteroalkinyl; unsubstituted or mono- or poly-substituted cycloalkyl or cycloalkenyl; unsubstituted or mono- or poly-substituted heterocycloalkyl or heterocycloalkenyl; unsubstituted or mono- or poly-substituted aryl; unsubstituted or mono- or poly-substituted heteroaryl; unsubstituted or mono- or poly-substituted -(alkylene)-aryl, -(alkenylene)-aryl, (alkinylene)-aryl, (heteroalkylene)-aryl or -(heteroalkenylene)-aryl; or unsubstituted or mono- or poly-substituted -(alkylene)-heteroaryl, -(alkenylene)-heteroaryl, -(alkinylene)-heteroaryl, -(heteroalkylene)-heteroaryl or -(heteroalkenylene)-heteroaryl;
R⁹ represents H, —C(═O)—OH; —C(═O)—H; —C(═O)—R¹⁵; —C(═O)—O—R¹⁶; —C(═O)—NH₁₂; —C(═O)—NH—R¹⁷; —C(═S)—NH—R¹⁷; —C(═O)—NR¹⁸R¹⁹; —C(═S)—NR¹⁸R¹⁹; —S(═O)—R²⁰; —S(═O)₂—R²¹; unsubstituted or mono- or poly-substituted alkyl, alkenyl or alkinyl; unsubstituted or mono- or poly-substituted heteroalkyl, heteroalkenyl or heteroalkinyl; unsubstituted or mono- or poly-substituted cycloalkyl or cycloalkenyl; unsubstituted or mono- or poly-substituted heterocycloalkyl or heterocycloalkenyl; unsubstituted or mono- or poly-substituted -(alkylene)-cyloalkyl, -(alkenylene)-cycloalkyl, -(alkinylene)-cycloalkyl, -(alkylene)-cycloalkenyl, -(alkenylene)-cycloalkenyl or -(alkinylene)-cycloalkenyl; unsubstituted or mono- or poly-substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or mono- or poly-substituted -(alkylene)-heterocycloalkyl, -(alkenylene)-heterocycloalkyl, -(alkinylene)-heterocycloalkyl, -(alkylene)-heterocycloalkenyl, -(alkenylene)-heterocycloalkenyl or -(alkinylene)-heterocycloalkenyl; unsubstituted or mono- or poly-substituted -(heteroalkylene)-heterocycloalkyl, (heteroalkenylene)-heterocycloalkyl-(heteroalkylene)-heterocycloalkenyl or -(heteroalkenylene)-heterocycloalkenyl; unsubstituted or mono- or poly-substituted aryl; unsubstituted or mono- or poly-substituted heteroaryl; unsubstituted or mono- or poly-substituted -(alkylene)-aryl, -(alkenylene)-aryl, (alkinylene)-aryl, (heteroalkylene)-aryl or -(heteroalkenylene)-aryl; or unsubstituted or mono- or poly-substituted -(alkylene)-heteroaryl, -(alkenylene)-heteroaryl, -(alkinylene)-heteroaryl, -(heteroalkylene)-heteroaryl or -(heteroalkenylene)-heteroaryl;
R¹⁰ represents —C(═O)—OH; —C(═O)—H; —C(═O)—R¹⁵; —C(═O)—O—R¹⁶; —C(═O)—NH₂; —C(═O)—NH—R¹⁷; —C(═S)—NH—R¹⁷; —C(═O)—NR¹⁸R¹⁹; —C(═S)—NR¹⁸R¹⁹; —S(═O)—R²⁰; —S(═O)₂—R²¹; alkyl, alkenyl or alkinyl, which is respectively substituted with 1, 2, 3, 4 or 5 substituents independently selected from the group consisting of F, Cl, Br, I, —NO₂, —CN, —SH, —NH₂, —N(C₁₋₅)-alkyl)₂, —N(C₁₋₅-alkyl)(phenyl), —N(C₁₋₅-alkyl)(CH₂-phenyl), —N(C₁₋₅-alkyl)(CH₂—CH₂-phenyl), NH—C(═O)—O—C₁₋₅-alkyl, —C(═O)—H, —C(═O)—C₁₋₅-alkyl, —C(═O)-phenyl, —C(═S)—C₁₋₅-alkyl, —C(═S)-phenyl, —C(═O)—OH, —C(═O)—O—C₁₋₅-alkyl, —C(═O)—O-phenyl, C(═O)—NH₂, —C(═O)—NH—C₁₋₅-alkyl, —C(═O)—N(C₁₋₅-alkyl)₂, —S(═O)—C₁₋₅-alkyl, —S(═O)-phenyl, —S(═O)₂-phenyl, —S(═O)₂—NH₂ and —SO₃H, wherein the phenyl residues may be unsubstituted or substituted with 1, 2, 3, 4 or 5 substituents independently selected from the group consisting of F, Cl, Br, I, —CN, CF₃, —OH, —NH₂, O—CF₃, —SH, —O—CH₃, —O—C₂H₅, —O—C₃H₇, methyl, ethyl, n-propyl, isopropyl, n-butyl, 2-butyl, isobutyl and tert-butyl; unsubstituted or mono- or poly-substituted heteroalkenyl or heteroalkinyl; unsubstituted or mono- or poly-substituted cycloalkyl or cycloalkenyl; unsubstituted or mono- or poly-substituted heterocycloalkyl or heterocycloalkenyl; unsubstituted or mono- or poly-substituted -(alkylene)-cyloalkyl, -(alkenylene)-cyclo-alkyl, -(alkinylene)-cycloalkyl, -(alkylene)-cycloalkenyl, -(alkenylene)-cycloalkenyl or -(alkinylene)-cycloalkenyl; unsubstituted or mono- or poly-substituted -(hetero-alkylene)-cycloalkyl, -(heteroalkenylene)-cycloalkyl, -(heteroalkylene)-cycloalkenyl or -(heteroalkenylene)-cycloalkenyl; unsubstituted or mono- or poly-substituted -(alkylene)-heterocycloalkyl, -(alkenylene)-heterocycloalkyl, -(alkinylene)-heterocycloalkyl, -(alkylene)-heterocycloalkenyl, -(alkenylene)-heterocycloalkenyl or -(alkinylene)-heterocycloalkenyl; unsubstituted or mono- or poly-substituted -(heteroalkylene)-heterocycloalkyl, (heteroalkenylene)-heterocycloalkyl, -(heteroalkylene)-heterocycloalkenyl or -(heteroalkenylene)-heterocycloalkenyl; unsubstituted or mono- or poly-substituted aryl; unsubstituted or mono- or poly-substituted heteroaryl; unsubstituted or mono- or poly-substituted -(alkylene)-aryl, -(alkenylene)-aryl, (alkinylene)-aryl, (heteroalkylene)-aryl or -(heteroalkenylene)-aryl; or unsubstituted or mono- or poly-substituted -(alkylene)-heteroaryl, -(alkenylene)-heteroaryl, -(alkinylene)-heteroaryl, -(heteroalkylene)-heteroaryl or -(heteroalkenylene)-heteroaryl; or
R⁹ and R¹⁰ together with the nitrogen atom to which they are bound form a residue selected from the group consisting of imidazolidinyl, azepanyl, pyrrolyl, diazepanyl, isothioazolidinyl, (2,3)-dihydro-1H-isoindolyl, indolinyl, octahydro-1H-isoindolyl, (2,3)-dihydropyrrolyl, (2,5)-dihydropyrrolyl, 8-azaspiro[4.5]decyl, 8-azaspiro[4.5]-decane-7,9-dionyl, isoindoline-1,3-dionyl, pyrrolidine-2,5-dionyl, morpholine-3,5-dionyl, 1H-pyrrole-2,5-dionyl, piperidine-2,6-dionyl, hexahydro-2H-isoindoline-1,3-dionyl, pyrrolidine-2-onyl and 1,1-dioxo-1λ⁶-isothiazolidinyl, each of which may be unsubstituted or optionally substituted with 1, 2, 3, 4 or 5 substituents independently selected from the group consisting of F, Cl, Br, I, —CN, CF₃, —OH, —NH₂, O—CF₃, —SH, —O—C₁₋₅-alkyl, —O-phenyl, —O—CH₂-phenyl, —(CH₂)—O—C₁₋₅-alkyl, —S—C₁₋₅-alkyl, —S-phenyl, —S—CH₂-phenyl, —C₁₋₅-alkyl, —C₂₋₅-alkenyl, —C₂₋₅-alkinyl, —C≡C—Si(CH₃)₃, —C≡C—Si(C₂H₅)₃, —C(═O)—O—C₁₋₅-alkyl, —C(═O)—CF₃, —S(═O)₂—C₁₋₅-alkyl, —S(═O)—C₁₋₅-alkyl, —S(═O)₂-phenyl, thioxo (═S), —N(C₁₋₅-alkyl)₂, —N(H)(C₁₋₅-alkyl), —NO₂, —S—CF₃, —C(═O)—OH, —NH—S(═O)₂—C₁₋₅-alkyl, —NH—C(═O)—C₁₋₅-alkyl, —NH—C(═O)—CF₃, —C(═O)—H, —C(═O)—C₁₋₅-alkyl, —C(═O)—NH₂, —C(═O)—N(C₁₋₅-alkyl)₂, —C(═O)—N(H)(C₁₋₅-alkyl) and phenyl, wherein the phenyl residues can be respectively unsubstituted or substituted with 1, 2, 3, 4 or 5 substituents independently selected from the group consisting of F, Cl, Br, I, —CN, —CF₃, —OH, —NH₂, —O—CF₃, —SH, —O—C₁₋₅-alkyl, —O-phenyl, —O—CH₂-phenyl, —(CH₂)—O—C₁₋₅-alkyl, —S—C₁₋₅-alkyl, —S-phenyl, —S—CH₂-phenyl, —C₁₋₅-alkyl, —C₂₋₅-alkenyl, —C₂₋₅-alkinyl, —C≡C—Si(CH₃)₃, —C≡C—Si(C₂H₅)₃, —C(═O)—O—C₁₋₅-alkyl and —C(═O)—CF₃, wherein the abovementioned phenyl residues may be unsubstituted or substituted with 1, 2, 3, 4 or 5 substituents independently selected from the group consisting of F, Cl, Br, I, —CN, —CF₃, —OH, —NH₂, —O—CF₃, —SH, —O—CH₃, —O—C₂H₅, —O—C₃H₇, methyl, ethyl, n-propyl, isopropyl, n-butyl, 2-butyl, isobutyl and tert-butyl; and
if R¹ represents a residue selected from the group consisting of methyl, ethyl, n-propyl, isopropyl, n-butyl, isobutyl, 2-butyl, tert-butyl and cyclopropyl, then R¹⁰ may additionally represent H, unsubstituted alkyl or unsubstituted or mono- or poly-substituted heteroalkyl;
R¹¹, R¹², R¹³ and R¹⁴ each independently represent H; F; Cl; Br; I; —NO₂; —CN, —NH₂, —OH; —SH; —C(═O)—OH; —C(═O)—H; —C(═O)—R¹⁵; —C(═O)—O—R¹⁶; —C(═O)—NH₂; —C(═O)—NH—R¹⁷; —C(═S)—NH—R¹⁷; —C(═O)—NR¹⁸R¹⁹; —C(═S)—NR¹⁸R¹⁹; —S(═O)—R²⁰; —S(═O)₂—R²¹; —NH—R²²; —NR²³R²⁴; —O—R²⁵; —S—R²⁶; —O—C(═O)—R²⁷; —NH—C(═O)—H; —NH—C(═O)—R²⁸; —NR²⁹—C(═O)—R³⁰; —NH—C(═O)—NH—R³¹; —NH—C(═S)—NH—R³²; —NH—S(═O)₂—R³³; —NR³⁴—S(═O)₂—R³⁵; unsubstituted or mono- or poly-substituted alkyl, alkenyl or alkinyl; unsubstituted or mono- or poly-substituted heteroalkyl, heteroalkenyl or heteroalkinyl; unsubstituted or mono- or poly-substituted cycloalkyl or cycloalkenyl; unsubstituted or mono- or poly-substituted heterocycloalkyl or heterocycloalkenyl; unsubstituted or mono- or poly-substituted aryl; unsubstituted or mono- or poly-substituted heteroaryl; unsubstituted or mono- or poly-substituted -(alkylene)-aryl, -(alkenylene)-aryl, (alkinylene)-aryl, (heteroalkylene)-aryl or -(heteroalkenylene)-aryl; or unsubstituted or mono- or poly-substituted -(alkylene)-heteroaryl, -(alkenylene)-heteroaryl, -(alkinylene)-heteroaryl, -(heteroalkylene)-heteroaryl or -(heteroalkenylene)-heteroaryl;
R¹⁵ represents —C(═O)—O—R¹⁶; unsubstituted or mono- or poly-substituted alkyl, alkenyl or alkinyl; unsubstituted or mono- or poly-substituted heteroalkyl, heteroalkenyl or heteroalkinyl; unsubstituted or mono- or poly-substituted cycloalkyl or cycloalkenyl; unsubstituted or mono- or poly-substituted heterocycloalkyl or heterocycloalkenyl; unsubstituted or mono- or poly-substituted -(alkylene)-cyloalkyl, -(alkenylene)-cycloalkyl, or -(alkinylene)-cycloalkyl, -(alkylene)-cycloalkenyl, -(alkenylene)-cycloalkenyl or -(alkinylene)-cycloalkenyl; unsubstituted or mono- or poly-substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or mono- or poly-substituted -(alkylene)-heterocycloalkyl, -(alkenylene)-heterocycloalkyl, -(alkinylene)-heterocycloalkyl, -(alkylene)-heterocycloalkenyl, -(alkenylene)-heterocycloalkenyl or -(alkinylene)-heterocycloalkenyl; unsubstituted or mono- or poly-substituted -(heteroalkylene)-heterocycloalkyl, (heteroalkenylene)-heterocycloalkyl; -(heteroalkylene)-heterocycloalkenyl or -(heteroalkenylene)-heterocycloalkenyl; unsubstituted or mono- or poly-substituted aryl; unsubstituted or mono- or poly-substituted heteroaryl; unsubstituted or mono- or poly-substituted -(alkylene)-aryl, -(alkenylene)-aryl, (alkinylene)-aryl, (heteroalkylene)-aryl or -(heteroalkenylene)-aryl; or unsubstituted or mono- or poly-substituted -(alkylene)-heteroaryl, -(alkenylene)-heteroaryl, -(alkinylene)-heteroaryl, -(heteroalkylene)-heteroaryl or -(heteroalkenylene)-heteroaryl; and
R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², R³³, R³⁴ and R³⁵ each independently represent unsubstituted or mono- or poly-substituted alkyl, alkenyl or alkinyl; unsubstituted or mono- or poly-substituted heteroalkyl, heteroalkenyl or heteroalkinyl; unsubstituted or mono- or poly-substituted cycloalkyl or cycloalkenyl; unsubstituted or mono- or poly-substituted heterocycloalkyl or heterocycloalkenyl; unsubstituted or mono- or poly-substituted -(alkylene)-cyloalkyl, -(alkenylene)-cycloalkyl, -(alkinylene)-cycloalkyl, -(alkylene)-cycloalkenyl, -(alkenylene)-cycloalkenyl or -(alkinylene)-cycloalkenyl; unsubstituted or mono- or poly-substituted -(heteroalkylene)-cycloalkyl, -(heteroalkenylene)-cycloalkyl, -(heteroalkylene)-cycloalkenyl or -(heteroalkenylene)-cycloalkenyl; unsubstituted or mono- or poly-substituted -(alkylene)-heterocycloalkyl, -(alkenylene)-heterocycloalkyl, -(alkinylene)-heterocycloalkyl, -(alkylene)-heterocycloalkenyl, -(alkenylene)-heterocycloalkenyl or -(alkinylene)-heterocycloalkenyl; unsubstituted or mono- or poly-substituted -(heteroalkylene)-heterocycloalkyl, (heteroalkenylene)-heterocycloalkyl; -(hetero-alkylene)-heterocycloalkenyl or -(heteroalkenylene)-heterocycloalkenyl; unsubstituted or mono- or poly-substituted aryl; unsubstituted or mono- or poly-substituted heteroaryl; unsubstituted or mono- or poly-substituted -(alkylene)-aryl, -(alkenylene)-aryl, (alkinylene)-aryl, (heteroalkylene)-aryl or -(heteroalkenylene)-aryl; or unsubstituted or mono- or poly-substituted -(alkylene)-heteroaryl, -(alkenylene)-heteroaryl, -(alkinylene)-heteroaryl, -(heteroalkylene)-heteroaryl or -(heteroalkenylene)-heteroaryl;
wherein
the abovementioned alkyl residues may be straight-chain or branched and contain 1, 2, 3, 4, 5, 6, 7, 8, 9, 10, 11 or 12 carbon atoms as chain members;
the abovementioned alkenyl residues may be straight-chain or branched and contain 2, 3, 4, 5, 6, 7, 8, 9, 10, 11 or 12 carbon atoms as chain members;
the abovementioned alkinyl residues may be straight-chain or branched and contain 2, 3, 4, 5, 6, 7, 8, 9, 10, 11 or 12 carbon atoms as chain members;
the abovementioned heteroalkyl residues, heteroalkenyl residues and heteroalkinyl residues are respectively 2-, 3-, 4-, 5-, 6-, 7-, 8-, 9-, 10-, 11- or 12-membered;
the abovementioned heteroalkyl residues, heteroalkenyl residues and heteroalkinyl residues respectively have optionally 1, 2 or 3 heteroatom(s) independently selected from the group consisting of oxygen, sulfur and nitrogen as chain member(s);
unless otherwise indicated, the abovementioned alkyl residues, alkenyl residues, alkinyl residues, heteroalkyl residues, heteroalkenyl residues and heteroalkinyl residues may optionally be substituted with 1, 2, 3, 4 or 5 substituents independently selected from the group consisting of F, Cl, Br, I, —NO₂, —CN, —OH, —SH, —NH₂, —N(C₁₋₅-alkyl)₂, —N(C₁₋₅-alkyl)(phenyl), —N(C₁₋₅-alkyl)(CH₂-phenyl), —N(C₁₋₅-alkyl)(CH₂—CH₂-phenyl), —NH—C(═O)—O—C₁₋₅-alkyl, —C(═O)—H, —C(═O)—C₁₋₅-alkyl, —C(═O)-phenyl, —C(═S)—C₁₋₅-alkyl, —C(═S)-phenyl, —C(═O)—OH, —C(═O)—O—C₁₋₅-alkyl, —C(═O)—O-phenyl, —C(═O)—NH₂, —C(═O)—NH—C₁₋₅-alkyl, —C(═O)—N(C₁₋₅-alkyl)₂, —S(═O)—C₁₋₅-alkyl, —S(═O)-phenyl, —S(═O)₂-phenyl, —S(═O)₂—NH₂ and —SO₃H, wherein the phenyl residues may be unsubstituted or substituted with 1, 2, 3, 4 or 5 substituents independently selected from the group consisting of F, Cl, Br, I, —CN, —CF₃, —OH, —NH₂, —O—CF₃, —SH, —O—CH₃, —O—C₂H₅, —O—C₃H₇, methyl, ethyl, n-propyl, isopropyl, n-butyl, 2-butyl, isobutyl and tert-butyl;
the abovementioned cycloalkyl residues respectively have 3, 4, 5, 6, 7, 8 or 9 carbon atoms as ring members;
the abovementioned cycloalkenyl residues respectively have 3, 4, 5, 6, 7, 8 or 9 carbon atoms as ring members;
the abovementioned heterocycloalkyl residues are respectively 3-, 4-, 5-, 6-, 7-, 8- or 9-membered;
the abovementioned heterocycloalkenyl residues are respectively 4-, 5-, 6-, 7-, 8- or 9-membered;
the abovementioned heterocycloalkyl residues and heterocycloalkenyl residues may each contain 1, 2 or 3 heteroatoms independently selected from the group consisting of oxygen, sulfur and nitrogen as ring members;
unless otherwise indicated, the abovementioned cycloalkyl residues, heterocycloalkyl residues, cycloalkenyl residues or heterocycloalkenyl residues may be unsubstituted or substituted with 1, 2, 3, 4 or 5 substituents independently selected from the group consisting of F, Cl, Br, I, —CN, —CF₃, —OH, —NH₂, —O—CF₃, —SH, —O—C₁₋₅-alkyl, —O-phenyl, —O—CH₂-phenyl, —(CH₂)—O—C₁₋₅-alkyl, —S—C₁₋₅-alkyl, —S-phenyl, —S—CH₂-phenyl, —C₁₋₅-alkyl, —C₂₋₅-alkenyl, —C₂₋₅-alkinyl, —C≡C—Si(CH₃)₃, —C≡C—Si(C₂H₅)₃, —C(═O)—O—C₁₋₅-alkyl, —C(═O)—CF₃, —S(═O)₂—C₁₋₅-alkyl, —S(═O)—C₁₋₅-alkyl, —S(═O)₂-phenyl, oxo (═O), thioxo (═S), —N(C₁₋₅-alkyl)₂, —N(H)(C₁₋₅-alkyl), —NO₂, —S—CF₃, —C(═O)—OH, —NH—S(═O)₂—C₁₋₅-alkyl, —NH—C(═O)—C₁₋₅-alkyl, —NH—C(═O)—CF₃, —C(═O)—H, —C(═O)—C₁₋₅-alkyl, —C(═O)—NH₂, —C(═O)—N(C₁₋₅-alkyl)₂, —C(═O)—N(H)(C₁₋₅-alkyl) and phenyl, wherein the phenyl residues may be unsubstituted or substituted with 1, 2, 3, 4 or 5 substituents independently selected from the group consisting of F, Cl, Br, I, —CN, —CF₃, —OH, —NH₂, —O—CF₃, —SH, —O—C₁₋₅-alkyl, —O-phenyl, —O—CH₂-phenyl, —(CH₂)—O—C₁₋₅-alkyl, —S—C₁₋₅-alkyl, —S-phenyl, —S—CH₂-phenyl, —C₁₋₅-alkyl, —C₂₋₅-alkenyl, —C₂₋₅-alkinyl, —C≡C—Si(CH₃)₃, —C≡C—Si(C₂H₅)₃, —C(═O)—O—C₁₋₅-alkyl and —C(═O)—CF₃, wherein the foregoing phenyl substituents may be further substituted with 1, 2, 3, 4 or 5 substituents independently selected from the group consisting of F, Cl, Br, I, —CN, —CF₃, —OH, —NH₂, —O—CF₃, —SH, —O—CH₃, —O—C₂H₅, —O—C₃H₇, methyl, ethyl, n-propyl, isopropyl, n-butyl, 2-butyl, isobutyl and tert-butyl;
the abovementioned alkylene residues may be straight-chain or branched and contain 1, 2, 3, 4, 5, 6, 7, 8, 9, 10, 11 or 12 carbon atoms as chain members;
the abovementioned alkenylene residues may be straight-chain or branched and contain 2, 3, 4, 5, 6, 7, 8, 9, 10, 11 or 12 carbon atoms as chain members;
the abovementioned alkinylene residues may be straight-chain or branched and contain 2, 3, 4, 5, 6, 7, 8, 9, 10, 11 or 12 carbon atoms as chain members;
the abovementioned heteroalkylene residues and heteroalkenylene residues are respectively 2-, 3-, 4-, 5-, 6-, 7-, 8-, 9-, 10-, 11- or 12-membered;
the abovementioned heteroalkylene residues and heteroalkenylene groups respectively have optionally 1, 2 or 3 heteroatom(s) independently selected from the group consisting of oxygen, sulfur and nitrogen (NH) as chain member(s);
the abovementioned alkylene, alkenylene, alkinylene, heteroalkylene or heteroalkenylene groups may be unsubstituted or substituted with 1, 2, 3, 4 or 5 substituents independently selected from the group consisting of phenyl, F, Cl, Br, I, —NO₂, —CN, —OH, —O-phenyl, —O—CH₂-phenyl, —SH, —S-phenyl, —S—CH₂-phenyl, NH₂, —N(C₁₋₅-alkyl)₂, —NH-phenyl, —N(C₁₋₅-alkyl)(phenyl), —N(C₁₋₅-alkyl)(CH₂-phenyl), —N(C₁₋₅-alkyl)(CH₂—CH₂-phenyl), —C(═O)—H, —C(═O)—C₁₋₅-alkyl, —C(═O)-phenyl, —C(═S)—C₁₋₅-alkyl, —C(═S)-phenyl, —C(═O)—OH, —C(═O)—O—C₁₋₅-alkyl, —C(═O)—O-phenyl, —C(═O)—NH₂, —C(═O)—NH—C₁₋₅-alkyl, —C(═O)—N(C₁₋₅-alkyl)₂, —S(═O)—C₁₋₅-alkyl, —S(═O)-phenyl, —S(═O)₂—C₁₋₅-alkyl, —S(═O)₂-phenyl, —S(═O)₂—NH₂ and —SO₃H, wherein the phenyl residues can respectively be substituted with 1, 2, 3, 4 or 5 substituents independently selected from the group consisting of F, Cl, Br, I, —CN, —NO₂, —OH, —SH, —NH₂, —C(═O)—OH, —C₁₋₅-alkyl, —(CH₂)—O—C₁₋₅-alkyl, —C₂₋₅-alkenyl, —C₂₋₅-alkinyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂ and —S—CH₂F;
the abovementioned aryl residues may be mono- or bicyclic and contain 6, 10 or 14 carbon atoms;
the abovementioned heteroaryl residues are mono-, bi- or tricyclic and are 5-, 6-, 7-, 8-, 9-, 10-, 11-, 12-, 13- or 14-membered;
the abovementioned 5- to 14-membered heteroaryl residues contain 1, 2, 3, 4 or 5 heteroatoms independently selected from the group consisting of oxygen, sulfur and nitrogen as ring members;
the abovementioned aryl residues and heteroaryl residues may be unsubstituted or substituted with 1, 2, 3, 4 or 5 substituents independently selected from the group consisting of F, Cl, Br, I, —CN, —NO₂, —OH, —SH, —NH₂, —C(═O)—OH, —C₁₋₅-alkyl, —(CH₂)—O—C₁₋₅-alkyl, —C₂₋₅-alkenyl, —C₂₋₅-alkinyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, —S(═O)₂—C₁₋₅-alkyl, —S(═O)—C₁₋₅-alkyl, —NH—C₁₋₅-alkyl, N(C₁₋₅-alkyl)₂, —C(═O)—O—C₁₋₅-alkyl, —C(═O)—H, —C(═O)—C₁₋₅-alkyl, —CH₂—O—C(═O)-phenyl, —O—C(═O)-phenyl, —NH—S(═O)₂—C₁₋₅-alkyl, —NH—C(═O)—C₁₋₅-alkyl, —C(═O)—NH₂, —C(═O)—NH—C₁₋₅-alkyl, —C(═O)—N(C₁₋₅-alkyl)₂, cyclopropyl, cyclobutyl, cyclopentyl, cyclohexyl, piperazinyl, pyrrolidinyl, piperidinyl, pyrazolyl, phenyl, furyl (furanyl), thiazolyl, thiadiazolyl, thienyl, benzyl and phenethyl, wherein the cyclic substituents or the cyclic residues of these substituents can themselves be substituted with optionally 1, 2, 3, 4 or 5 substituents independently selected from the group consisting of F, Cl, Br, I, —CN, —NO₂, —OH, —SH, —NH₂, —C(═O)—OH, —C₁₋₅-alkyl, —(CH₂)—O—C₁₋₅-alkyl, —C₂₋₅-alkenyl, —C₂₋₅-alkinyl, —C≡C—Si(CH₃)₃, —C≡C—Si(C₂H₅)₃, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂ and —S—CH₂F;
the abovementioned aryl residues, heteroaryl residues, cycloalkyl residue, heterocycloalkyl residue, cycloalkenyl residue or heterocycloalkenyl residue optionally may be anellated with an unsubstituted or mono- or poly-substituted mono- or bicyclic ring system;
a mono- or bicyclic ring system is understood to mean mono- or bicyclic hydrocarbon residues, which may be saturated, unsaturated or aromatic; wherein the rings of the mono- or bicyclic ring systems may each be 4-, 5- or 6-membered, and may each contain 0, 1, 2, 3, 4 or 5 heteroatoms as ring members independently selected from the group consisting of oxygen, nitrogen and sulfur, and
the abovementioned mono- or bicyclic ring systems may be unsubstituted or substituted with 1, 2, 3, 4 or 5 substituents independently selected from the group consisting of F, Cl, Br, I, —CN, —NO₂, —OH, —SH, —NH₂, oxo (═O), thioxo (═S), —C(═O)—OH, C₁₋₅-alkyl, —C₂₋₅-alkenyl, —C₂₋₅-alkinyl, —C≡C—Si(CH₃)₃, —C≡C—Si(C₂Hs)₃, —(CH₂)—O—C₁₋₅-alkyl, —S—C₁₋₅-alkyl, —S-phenyl, —S—CH₂-phenyl, —O—C₁₋₅-alkyl, —O-phenyl, —O—CH₂-phenyl, —CF₃, —CHF₂, —CH₂F, —O—CF₃, —O—CHF₂, —O—CH₂F, —C(═O)—CF₃, —S—CF₃, —S—CHF₂, —S—CH₂F, —S(═O)₂-phenyl, —S(═O)₂—C₁₋₅-alkyl, —S(═O)—C₁₋₅-alkyl, —NH—C₁₋₅-alkyl, N(C₁₋₅-alkyl)(C₁₋₅-alkyl), —C(═O)—O—C₁₋₅-alkyl, —C(═O)—H, —C(═O)—C₁₋₅-alkyl, —CH₂—O—C(═O)-phenyl, —O—C(═O)-phenyl, —NH—S(═O)₂—C₁₋₅-alkyl, —NH—C(═O)—C₁₋₅-alkyl, —C(═O)—NH₂, —C(═O)—NH—C₁₋₅-alkyl, —C(═O)—N(C₁₋₅-alkyl)₂, pyrazolyl, phenyl, furyl, thiadiazolyl, thiophenyl and benzyl, wherein the abovementioned C₁₋₅-alkyl residues may be straight-chain or branched and the cyclic substituents or the cyclic residues of these substituents may be unsubstituted or substituted with 1, 2, 3, 4 or 5 substituents independently selected from the group consisting of F, Cl, Br, I, —CN, —CF₃, —OH, —NH₂, —O—CF₃, —SH, —O—C₁₋₅-alkyl, —O-phenyl, —O—CH₂-phenyl, —(CH₂)—O—C₁₋₅-alkyl, —S—C₁₋₅-alkyl, —S-phenyl, —S—CH₂-phenyl, —C₁₋₅-alkyl, —C₂₋₅-alkenyl, —C₂₋₅-alkinyl, —C≡C—Si(CH₃)₃, —C≡C—Si(C₂H₅)₃, —C(═O)—O—C₁₋₅-alkyl and —C(═O)—CF₃; and
the abovementioned cycloalkyl residues, heterocycloalkyl residues, cycloalkenyl residues or heterocycloalkenyl residue can form a spirocyclic residue with a further cycloalkyl residue, heterocycloalkyl residue, cycloalkenyl residue or heterocycloalkenyl residue via a common carbon atom in the rings.

6. A compound as claimed in claim 1, wherein T, U, V and optionally W together with two carbon atoms form a ring selected from the group consisting of:

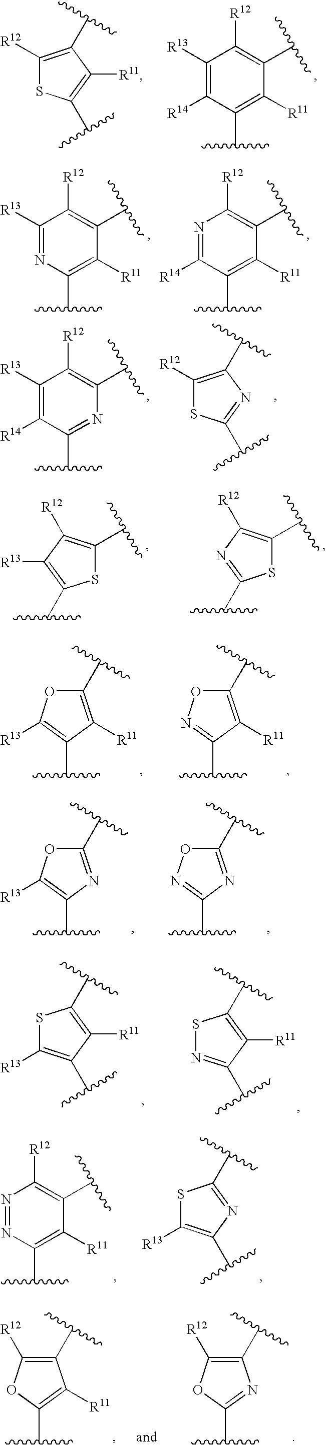

7. A compound as claimed in claim 1, wherein:
R¹ and R² each independently represent H; —C(═O)—OH; —C(═O)—H; —C(═O)—R¹⁵; —C(═O)—O—R¹⁶; —C(═O)—NH₂; —C(═O)—NH—R¹⁷; —C(═S)—NH—R¹⁷; —C(═O)—NR¹⁸R¹⁹; —C(═S)—NR¹⁸R¹⁹; —S(═O)—R²⁰; —S(═O)₂—R²¹; C₁₋₆-alkyl, C₂₋₆-alkenyl or C₂₋₆-alkinyl, each of which may be unsubstituted or substituted with 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; 2- to 6-membered heteroalkyl, 2- to 6-membered heteroalkenyl or 2- to 6-membered heteroalkinyl, each of which may be unsubstituted or substituted with 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃, and contains 1 or 2 heteroatoms independently selected from the group consisting of oxygen, sulfur and nitrogen as chain members; C₃₋₇-cycloalkyl, C₅₋₆-cycloalkenyl, 5- to 7-membered heterocycloalkyl or 5- to 7-membered heterocycloalkenyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅, and optionally may contain 1 or 2 heteroatoms independently selected from the group consisting of oxygen, sulfur and nitrogen as ring members; or a residue selected from the group consisting of phenyl, naphthyl and anthracenyl, which optionally may be bonded by a C₁₋₃-alkylene, C₂₋₃-alkenylene or C₂₋₃-alkinylene group and may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, piperazinyl, pyrrolidinyl, piperidinyl, —C(═O)—NH—CH₃, —NH—C(═O)—CH₃, —NH—C(═O)—C₂H₅, —C(═O)—O—CH₃ and —C(═O)—O—C₂H₅; or
R¹ and R² together with the nitrogen atom to which they are bound represent 5- to 7-membered heterocycloalkyl or 5- to 7-membered heterocycloalkenyl, which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅, and may contain 1 or 2 further heteroatoms independently selected from the group consisting of oxygen, sulfur and nitrogen as ring members.

8. A compound as claimed in claim 1, wherein
R³ represents H; F; Cl; Br; I; —NO₂; —CN; —NH₂; —OH; —SH; —C(═O)—OH; —C(═O)—H; —C(═O)—R¹⁵; —C(═O)—O—R¹⁶; —C(—O)—NH₂; —C(═O)—NH—R¹⁷; —C(═S)—NH—R¹⁷; —C(═O)—NR¹⁸R¹⁹; —C(═S)—NR¹⁸R¹⁹; —NH—R²²; —NR²³R²⁴; —O—R²⁵; —S—R²⁶; C₁₋₆-alkyl, C₂₋₆-alkenyl or C₂₋₆-alkinyl, each of which may be unsubstituted or substituted with 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃ and —C(═O)—O—C₂H₅; C₃₋₇-cycloalkyl, C₅₋₆-cycloalkenyl, 5- to 7-membered heterocycloalkyl or 5- to 7-membered heterocycloalkenyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅, and which may contain 1 or 2 heteroatoms independently selected from the group consisting of oxygen, sulfur and nitrogen as ring members; or a residue selected from the group consisting of phenyl, pyrrolyl, furanyl, thienyl, pyrazolyl, imidazolyl, thiazolyl, thiadiazolyl, triazolyl, oxazolyl, oxadiazolyl, isoxazolyl, pyridinyl, pyridazinyl, pyrimidinyl, pyrazinyl and pyranyl, each of which may be bonded by a C₁₋₃-alkylene, C₂₋₃-alkenylene or C₂₋₃-alkinylene group and which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅.

9. A compound as claimed in claim 1, wherein:
R⁴, R⁵ and R⁶ each independently represent H; F; Cl; Br; I; —NO₂; —CN; —NH₂; —OH; —SH; —C(═O)—OH; —C(═O)—H; —C(═O)—NH₂; —NH—R²²; —NR²³R²⁴; —O—R²⁵; —S—R²⁶; or C₁₋₆-alkyl, C₂₋₆-alkenyl or C₂₋₆-alkinyl, each of which may be unsubstituted or substituted with 1, 2, 3, 4 or 5 substituents independently selected from the group consisting of F, Cl, Br, I, —NO₂, —CN, —OH, —SH and —NH₂.

10. A compound as claimed in claim 1, wherein:
R⁷ and R⁸ each independently represent H; F; Cl; Br; I; —NO₂; —CN; —NH₂; —OH; —SH; —C(═O)—OH; —C(═O)—H; —C(═O)—NH₂; —NH—R²²; —NR²³R²⁴; —O—R²⁵; —S—R²⁶; or C₁₋₆-alkyl, C₂₋₆-alkenyl or C₂₋₆-alkinyl, each of which may be unsubstituted or substituted with 1, 2, 3, 4 or 5 substituents independently selected from the group consisting of F, Cl, Br, I, —NO₂, —CN, —OH, —SH and —NH₂.

11. A compound as claimed in claim 1, wherein:
R⁹ represents H; —C(═O)—OH; —C(═O)—H; —C(═O)—R¹⁵; —C(═O)—O—R¹⁶; —C(═O)—NH₂; —C(═O)—NH—R¹⁷; —C(═S)—NH—R¹⁷; —C(═O)—NR¹⁸R¹⁹; —C(═S)—NR¹⁸R¹⁹; —S(═O)—R²⁰; —S(═O)₂—R²¹; C₁₋₆-alkyl, C₂₋₆-alkenyl or C₂₋₆-alkinyl, each of which may be unsubstituted or substituted with 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; 2- to 6-membered heteroalkyl, 2- to 6-membered heteroalkenyl or 2- to 6-membered heteroalkinyl, each of which may be unsubstituted or substituted with 1, 2, 3, 4 or 5 substituents independently selected from the group consisting of F, Cl, Br, I, —NO₂, —CN, —OH, —SH, —NH₂, —N(CH₃)₂, —N(C₂H₅)₂ and —N(CH₃)(C₂H₅) and which may contain 1 or 2 heteroatoms independently selected from the group consisting of oxygen, sulfur and nitrogen as chain members; C₃₋₇-cycloalkyl or C₅₋₆-cycloalkenyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or a residue selected from the group consisting of phenyl, pyrrolyl, furanyl, thienyl, pyrazolyl, imidazolyl, thiazolyl, thiadiazolyl, triazolyl, oxazolyl, oxadiazolyl, isoxazolyl, pyridinyl, pyridazinyl, pyrimidinyl, pyrazinyl and pyranyl, each of which may optionally be bonded via a C₁₋₃-alkylene, C₂₋₃-alkenylene or C₂₋₃-alkinylene group and which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅; and
R¹⁰ represents —C(═O)—OH; —C(═O)—H; —C(═O)—R¹⁵; —C(═O)—O—R¹⁶; —C(═O)—NH₂; —C(═O)—NH—R¹⁷; —C(═S)—NH—R¹⁷; —C(═O)—NR¹⁸R¹⁹; —C(═S)—NR¹⁸R¹⁹; —S(═O)—R²⁰; —S(═O)₂—R²¹; C₁₋₆-alkyl, C₂₋₆-alkenyl or C₂₋₆-alkinyl, each of which may be unsubstituted or substituted with 1, 2, 3, 4 or 5 substituents independently selected from the group consisting of F, Cl, Br, I, —NO₂, —CN, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; 2- to 6-membered heteroalkenyl or 2- to 6-membered heteroalkinyl, each of which may be unsubstituted or substituted with 1, 2, 3, 4 or 5 substituents independently selected from the group consisting of F, Cl, Br, I, —NO₂, —CN, —OH, —SH, —NH₂, —N(CH₃)₂, —N(C₂H₅)₂ and —N(CH₃)(C₂H₅) and has 1 or 2 heteroatom(s) independently selected from the group consisting of oxygen, sulfur and nitrogen as chain members; C₃₋₇-cycloalkyl or C₅₋₆-cycloalkenyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or a residue selected from the group consisting of phenyl, pyrrolyl, furanyl, thienyl, pyrazolyl, imidazolyl, thiazolyl, thiadiazolyl, triazolyl, oxazolyl, oxadiazolyl, isoxazolyl, pyridinyl, pyridazinyl, pyrimidinyl, pyrazinyl and pyranyl, each of which may optionally be bonded via a C₁₋₃-alkylene, C₂₋₃-alkenylene or C₂₋₃-alkinylene group and which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅; and
if R¹ represents a residue selected from the group consisting of methyl, ethyl, n-propyl, isopropyl, n-butyl, isobutyl, 2-butyl, tert-butyl and cyclopropyl, then R¹⁰ may additionally represent H, unsubstituted C₁₋₆-alkyl or 2- to 6-membered heteroalkyl, each of which may be unsubstituted or substituted with 1, 2, 3, 4 or 5 substituents independently selected from the group consisting of F, Cl, Br, I, —NO₂, —CN, —OH, —SH, —NH₂, —N(CH₃)₂, —N(C₂H₅)₂ and —N(CH₃)(C₂H₅) and which may contain 1 or 2 heteroatoms independently selected from the group consisting of oxygen, sulfur and nitrogen as chain members; or
R⁹ and R¹⁰ together with the nitrogen atom to which they are bound represent a residue selected from the group consisting of imidazolidinyl, azepanyl, pyrrolyl, diazepanyl, isothioazolidinyl, (2,3)-dihydro-1H-isoindolyl, indolinyl, octahydro-1H-isoindolyl, (2,3)-dihydropyrrolyl, (2,5)-dihydropyrrolyl, 8-azaspiro[4.5]decyl, 8-azaspiro[4.5]-decane-7,9-dionyl, isoindoline-1,3-dionyl, pyrrolidine-2,5-dionyl, morpholine-3,5-dionyl, 1H-pyrrole-2,5-dionyl, piperidine-2,6-dionyl, hexahydro-2H-isoindoline-1,3-dionyl, pyrrolidine-2-onyl and 1,1-dioxo-1λ⁶-isothiazolidinyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃, —S—C₂H₅, —NH—C(═O)—CF₃ and phenyl, wherein the phenyl group may be unsubstituted or substituted with 1, 2, 3, 4 or 5 substituents independently selected from the group consisting of F, Cl, Br, I, —CN, —CF₃, —OH, —NH₂, —O—CF₃, —SH, —O—CH₃, —O—C₂H₅, —O—C₃H₇, methyl, ethyl, n-propyl, isopropyl, n-butyl, 2-butyl, isobutyl and tert-butyl.

12. A compound as claimed in claim 1, wherein:
R¹¹, R¹², R¹³ and R¹⁴ each independently represent H; F; Cl; Br; I; —NO₂; —CN; —NH₂; —OH; —SH; —C(═O)—OH; —C(═O)—H; —C(═O)—NH₂; —NH—R²²; —NR²³R²⁴; —O—R²⁵; —S—R²⁶; or C₁₋₆-alkyl, C₂₋₆-alkenyl or C₂₋₆-alkinyl, each of which may be unsubstituted or substituted with 1, 2, 3, 4 or 5 substituents independently selected from the group consisting of F, Cl, Br, I, —NO₂, —CN, —OH, —SH and —NH₂.

13. A compounds as claimed in claim 1, wherein:
R¹⁵ represents —C(═O)—O—R¹⁶; C₁₋₆-alkyl, C₂₋₆-alkenyl or C₂₋₆-alkinyl, each of which may be unsubstituted or substituted with 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(—O)—O—C(CH₃)₃; 2- to 6-membered heteroalkyl, 2- to 6-membered heteroalkenyl or 2- to 6-membered heteroalkinyl, each of which may be unsubstituted or substituted with 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃ and which contains 1 or 2 heteroatoms independently selected from the group consisting of oxygen, sulfur and nitrogen as chain members; C₃₋₇-cycloalkyl, C₅₋₆-cycloalkenyl, 5- to 7-membered heterocycloalkyl or 5- to 7-membered heterocycloalkenyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅ and which may contain 1 or 2 heteroatoms independently selected from the group consisting of oxygen, sulfur and nitrogen as ring members; or represents a residue selected from the group consisting of phenyl, naphthyl, anthracenyl, pyrrolyl, indolyl, furanyl, benzo[b]furanyl, thienyl, benzo[b]thiophenyl, benzo[d]thiazolyl, pyrazolyl, imidazolyl, thiazolyl, thiadiazolyl, triazolyl, oxazolyl, oxadiazolyl, isoxazolyl, pyridinyl, pyridazinyl, pyrimidinyl, pyrazinyl, pyranyl, indazolyl, quinolinyl, isoquinolinyl and quinazolinyl, which can respectively be bonded by means of a C₁₋₃-alkylene-, C₂₋₃-alkenylene- or C₂₋₃-alkinylene group or 2- or 3-membered heteroalkylene; which has 1 heteroatom selected from the group consisting of oxygen, sulfur and nitrogen (NH) as chain member, and/or is unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅.

14. A compounds as claimed in claim 1, wherein:
R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², R³³, R³⁴ and R³⁵ each independently represent C₁₋₆-alkyl, C₂₋₆-alkenyl or C₂₋₆-alkinyl, each of which may be unsubstituted or substituted with 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; C₃₋₇-cycloalkyl or C₅₋₆-cycloalkenyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or a residue selected from the group consisting of phenyl, pyrrolyl, furanyl, thienyl, pyrazolyl, imidazolyl, thiazolyl, thiadiazolyl, triazolyl, oxazolyl, oxadiazolyl, isoxazolyl, pyridinyl, pyridazinyl, pyrimidinyl, pyrazinyl and pyranyl, each of which may optionally be bonded via a C₁₋₃-alkylene, C₂₋₃-alkenylene or C₂₋₃-alkinylene group and which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅.

15. A compound as claimed in claim 1, wherein T, U, V and optionally W together with two carbon atoms form a ring selected from the group consisting of: 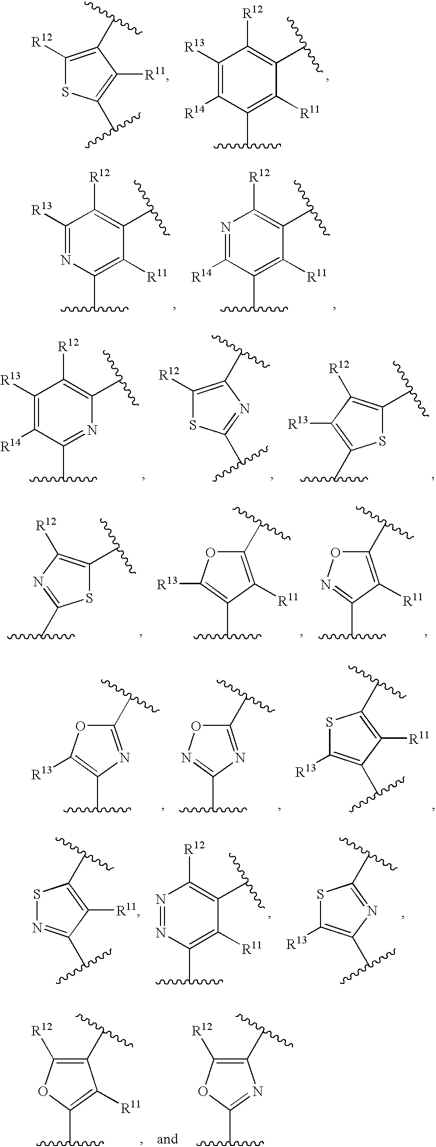
R¹ and R² each independently represent H; —C(═O)—OH; —C(═O)—H; —C(═O)—R¹⁵; —C(═O)—O—R¹⁶; —C(═O)—NH₂; —C(═O)—NH—R¹⁷; —C(═S)—NH—R¹⁷; —C(═O)—NR¹⁸R¹⁹; —C(═S)—NR¹⁸R¹⁹; —S(═O)—R²⁰; —S(═O)₂—R²¹; C₁₋₆-alkyl, C₂₋₆-alkenyl or C₂₋₆-alkinyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; 2- to 6-membered heteroalkyl, 2- to 6-membered heteroalkenyl or 2- to 6-membered heteroalkinyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃ and has 1 or 2 heteroatom(s) independently selected from the group consisting of oxygen, sulfur and nitrogen (NH) as chain member(s); C₃₋₇-cycloalkyl, C₅₋₆-cycloalkenyl, 5- to 7-membered heterocycloalkyl or 5- to 7-membered heterocycloalkenyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅ and can optionally have 1 or 2 heteroatom(s) independently selected from the group consisting of oxygen, sulfur and nitrogen (NH) as ring member(s); or represent a residue selected from the group consisting of phenyl, naphthyl and anthracenyl, which can respectively be bonded by means of a C₁₋₃-alkylene, C₂₋₃-alkenylene or C₂₋₃-alkinylene group and/or is unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, piperazinyl, pyrrolidinyl, piperidinyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅; or
R¹ and R² together with the nitrogen atom to which they are bound represent 5- to 7-membered heterocycloalkyl or 5- to 7-membered heterocycloalkenyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅, and which may contain 1 or 2 further heteroatoms independently selected from the group consisting of oxygen, sulfur and nitrogen as ring members;
R³ represents H; F; Cl; Br; I; —NO₂; —CN; —NH₂; —OH; —SH; —C(═O)—OH; —C(═O)—H; —C(═O)—R¹⁵; —C(═O)—O—R¹⁶; —C(═O)—NH₂; —C(═O)—NH—R¹⁷; —C(═S)—NH—R¹⁷; —C(═O)—NR¹⁸R¹⁹; —C(═S)—NR¹⁸R¹⁹; —NH—R²²; —NR²³R²⁴; —O—R²⁵; —S—R²⁶; C₁₋₆-alkyl, C₂₋₆-alkenyl or C₂₋₆-alkinyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃ and —C(═O)—O—C₂H₅; C₃₋₇-cycloalkyl, C₅₋₆-cycloalkenyl, 5- to 7-membered heterocycloalkyl or 5- to 7-membered heterocycloalkenyl, which is respectively unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅, and can optionally have 1 or 2 heteroatoms independently selected from the group consisting of oxygen, sulfur and nitrogen as ring members; or represents a residue selected from the group consisting of phenyl, pyrrolyl, furanyl, thienyl, pyrazolyl, imidazolyl, thiazolyl, thiadiazolyl, triazolyl, oxazolyl, oxadiazolyl, isoxazolyl, pyridinyl, pyridazinyl, pyrimidinyl, pyrazinyl and pyranyl, which can respectively be bonded by means of a C₁₋₃-alkylene, C₂₋₃-alkenylene or C₂₋₃-alkinylene group and/or be unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
R⁴, R⁵ and R⁶ each independently represent H; F; Cl; Br; I; —NO₂; —CN; —NH₂; —OH; —SH; —C(═O)—OH; —C(═O)—H; —C(═O)—NH₂; —NH—R²²; —NR²³R²⁴; —O—R²⁵; —S—R²⁶; or C₁₋₆-alkyl, C₂₋₆-alkenyl or C₂₋₆-alkinyl, which is respectively unsubstituted or substituted with optionally 1, 2, 3, 4 or 5 substituents independently selected from the group consisting of F, Cl, Br, I, —NO₂, —CN, —OH, —SH and —NH₂;
R⁷ and R⁸ each independently represent H; F; Cl; Br; I; —NO₂; —CN; —NH₂; —OH; —SH; —C(═O—O)—OH; —C(═O)—H; —C(═O)—NH₂; —NH—R²²; —NR²³R²⁴; —O—R²⁵; —S—R²⁶; or C₁₋₆-alkyl, C₂₋₆-alkenyl or C₂₋₆-alkinyl, each of which may be unsubstituted or substituted with 1, 2, 3, 4 or 5 substituents independently selected from the group consisting of F, Cl, Br, I, —NO₂, —CN, —OH, —SH and —NH₂;
R⁹ represents H; —C(═O)—OH; —C(═O)—H; —C(═O)—R¹⁵; —C(═O)—O—R¹⁶; —C(═O)—NH₂; —C(═O)—NH—R¹⁷; —C(═S)—NH—R¹⁷; —C(═O)—NR¹⁸R¹⁹; —C(═S)—NR¹⁸R¹⁹; —S(═O)—R²⁰; —S(═O)₂—R²¹; C₁₋₆-alkyl, C₂₋₆-alkenyl or C₂₋₆-alkinyl, which is respectively unsubstituted or substituted with optionally 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; 2- to 6-membered heteroalkyl, 2- to 6-membered heteroalkenyl or 2- to 6-membered heteroalkinyl, each of which may be unsubstituted or substituted with 1, 2, 3, 4 or 5 substituents independently selected from the group consisting of F, Cl, Br, I, —NO₂, —CN, —OH, —SH, —NH₂, —N(CH₃)₂, —N(C₂H₅)₂ and —N(CH₃)(C₂H₅) and which contains 1 or 2 heteroatoms independently selected from the group consisting of oxygen, sulfur and nitrogen as chain members; C₃₋₇-cycloalkyl or C₅₋₆-cycloalkenyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or represents a residue selected from the group consisting of phenyl, pyrrolyl, furanyl, thienyl, pyrazolyl, imidazolyl, thiazolyl, thiadiazolyl, triazolyl, oxazolyl, oxadiazolyl, isoxazolyl, pyridinyl, pyridazinyl, pyrimidinyl, pyrazinyl and pyranyl, which can respectively be bonded by means of a C₁₋₃-alkylene, C₂₋₃-alkenylene or C₂₋₃-alkinylene group and/or is unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
R¹⁰ represents —C(═O)—OH; —C(═O)—H; —C(═O)—R¹⁵; —C(═O)—O—R¹⁶; —C(═O)—NH₂; —C(═O)—NH—R¹⁷; —C(═S)—NH—R¹⁷; —C(═O)—NR¹⁸R¹⁹; —C(═S)—NR¹⁸R¹⁹; —S(═O)—R²⁰; —S(═O)₂—R²¹; C₁₋₆-alkyl, C₂₋₆-alkenyl or C₂₋₆-alkinyl, which is respectively substituted with 1, 2, 3, 4 or 5 substituents independently selected from the group consisting of F, Cl, Br, I, —NO₂, —CN, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; 2- to 6-membered heteroalkenyl or 2- to 6-membered heteroalkinyl, each of which may be unsubstituted or substituted with 1, 2, 3, 4 or 5 substituents independently selected from the group consisting of F, Cl, Br, I, —NO₂, —CN, —OH, —SH, —NH₂, —N(CH₃)₂, —N(C₂H₅)₂ and —N(CH₃)(C₂H₅) and has 1 or 2 heteroatom(s) independently selected from the group consisting of oxygen, sulfur and nitrogen as chain member(s); C₃₋₇-cycloalkyl or C₅₋₆-cycloalkenyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, NO₂, —CF₃, —O—CF₃, —S—CF₃, —SH, —S—CH₃ and —S—C₂H₅; or represents a residue selected from the group consisting of phenyl, pyrrolyl, furanyl, thienyl, pyrazolyl, imidazolyl, thiazolyl, thiadiazolyl, triazolyl, oxazolyl, oxadiazolyl, isoxazolyl, pyridinyl, pyridazinyl, pyrimidinyl, pyrazinyl and pyranyl, which can respectively be bonded by means of a C₁₋₃-alkylene, C₂₋₃-alkenylene or C₂₋₃-alkinylene group and/or is unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅; and
if R¹ represents a residue selected from the group consisting of methyl, ethyl, n-propyl, isopropyl, n-butyl, isobutyl, 2-butyl, tert-butyl and cyclopropyl, then R¹⁰ can additionally represent H, unsubstituted C₁₋₆-alkyl or 2- to 6-membered heteroalkyl, each of which may be unsubstituted or substituted with 1, 2, 3, 4 or 5 substituents independently selected from the group consisting of F, Cl, Br, I, —NO₂, —CN, —OH, —SH, —NH₂, —N(CH₃)₂, —N(C₂H₅)₂ and —N(CH₃)(C₂H₅) and which contains 1 or 2 heteroatoms independently selected from the group consisting of oxygen, sulfur and nitrogen as chain members; or
R⁹ and R¹⁰ together with the nitrogen atom to which they are bound represent a residue selected from the group consisting of imidazolidinyl, azepanyl, pyrrolyl, diazepanyl, isothioazolidinyl, (2,3)-dihydro-1H-isoindolyl, indolinyl, octahydro-1H-isoindolyl, (2,3)-dihydropyrrolyl, (2,5)-dihydropyrrolyl, 8-azaspiro[4.5]decyl, 8-azaspiro[4.5]-decane-7,9-dionyl, isoindoline-1,3-dionyl, pyrrolidine-2,5-dionyl, morpholine-3,5-dionyl, 1H-pyrrole-2,5-dionyl, piperidine-2,6-dionyl, hexahydro-2H-isoindoline-1,3-dionyl, pyrrolidine-2-onyl and 1,1-dioxo-1λ⁶-isothiazolidinyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃, —S—C₂H₅, —NH—C(═O)—CF₃ and phenyl;
R¹¹, R¹², R¹³ and R¹⁴ each independently represent H; F; Cl; Br; I; —NO₂; —CN; —NH₂; —OH; —SH; —C(═O)—OH; —C(═O)—H; —C(═O)—NH₂; —NH—R²²; —NR²³R²⁴; —O—R²⁵; —S—R²⁶; or C₁₋₆-alkyl, C₂₋₆-alkenyl or C₂₋₆-alkinyl, each of which may be unsubstituted or substituted with 1, 2, 3, 4 or 5 substituents independently selected from the group consisting of F, Cl, Br, I, —NO₂, —CN, —OH, —SH and —NH₂;
R¹⁵ represents —C(═O)—O—R¹⁶; C₁₋₆-alkyl, C₂₋₆-alkenyl or C₂₋₆-alkinyl, each of which may be unsubstituted or substituted with 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; 2- to 6-membered heteroalkyl, 2- to 6-membered heteroalkenyl or 2- to 6-membered heteroalkinyl, each of which may be unsubstituted or substituted with 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃ and contains 1 or 2 heteroatoms independently selected from the group consisting of oxygen, sulfur and nitrogen as chain members; C₃₋₇-cycloalkyl, C₅₋₆-cycloalkenyl, 5- to 7-membered heterocycloalkyl or 5- to 7-membered heterocycloalkenyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅ and which optionally may contain 1 or 2 heteroatoms independently selected from the group consisting of oxygen, sulfur and nitrogen as ring members; or a residue selected from the group consisting of phenyl, naphthyl, anthracenyl, pyrrolyl, indolyl, furanyl, benzo[b]furanyl, thienyl, benzo[b]thiophenyl, benzo[d]thiazolyl, pyrazolyl, imidazolyl, thiazolyl, thiadiazolyl, triazolyl, oxazolyl, oxadiazolyl, isoxazolyl, pyridinyl, pyridazinyl, pyrimidinyl, pyrazinyl, pyranyl, indazolyl, quinolinyl, isoquinolinyl and quinazolinyl, which can respectively be bonded by means of a C₁₋₃-alkylene, C₂₋₃-alkenylene or C₂₋₃-alkinylene group or 2- or 3-membered heteroalkylene; which has 1 heteroatom selected from the group consisting of oxygen, sulfur and nitrogen as chain member, and/or is unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
R¹⁶, R¹⁷, R¹⁸, R¹⁹, R²⁰, R²¹, R²², R²³, R²⁴, R²⁵ and R²⁶ each independently represent C₁₋₆-alkyl, C₂₋₆-alkenyl or C₂₋₆-alkinyl, each of which may be unsubstituted or substituted with 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; C₃₋₇-cycloalkyl or C₅₋₆-cycloalkenyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or represent a residue selected from the group consisting of phenyl, pyrrolyl, furanyl, thienyl, pyrazolyl, imidazolyl, thiazolyl, thiadiazolyl, triazolyl, oxazolyl, oxadiazolyl, isoxazolyl, pyridinyl, pyridazinyl, pyrimidinyl, pyrazinyl and pyranyl, which can be respectively bonded by means of a C₁₋₃-alkylene, C₂₋₃-alkenylene or C₂₋₃-alkinylene group and/or is unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅.

16. A compound as claimed in claim 1, wherein T, U, V and optionally W together with two carbon atoms form a ring selected from the group consisting of: 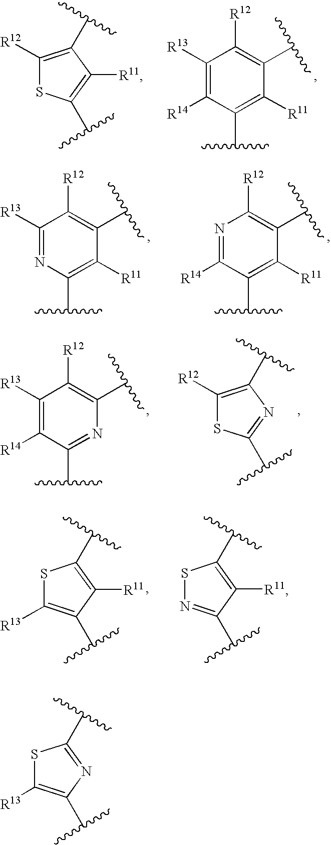
R¹ represents —C(═O)—R¹⁵; an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, each of which may be unsubstituted or substituted with 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; a residue selected from the group consisting of cyclopropyl, cyclobutyl, cyclopentyl, cyclohexyl, cycloheptyl, cyclopentenyl and cyclohexenyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or represents a residue selected from the group consisting of phenyl, naphthyl, benzyl and phenethyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
R² represents H; —C(═O)—R¹⁵; or an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, each of which may be unsubstituted or substituted with 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; or
R¹ and R² together with the nitrogen atom to which they are bound represent a residue selected from the group consisting of piperazinyl, piperidinyl, morpholinyl, thiomorpholinyl, pyrrolidinyl, azepanyl and diazepanyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —NO₂, —CF₃, —O—CF₃, —S—CF₃, —SH, —S—CH₃ and —S—C₂H₅;
R³ represents H; F; Cl; Br; I; —NO₂; —CN; —NH₂; —OH; —SH; —NH—R²²; —NR²³R²⁴; —O—R²⁵; —S—R²⁶; an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, each of which may be unsubstituted or substituted with 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃ and —C(═O)—O—C₂H₅; a residue selected from the group consisting of morpholinyl, piperazinyl, piperidinyl, pyrrolidinyl, azepanyl, diazepanyl, thiomorpholinyl, cyclopropyl, cyclobutyl, cyclopentyl, cyclohexyl, cycloheptyl, cyclopentenyl and cyclohexenyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —NO₂, —CF₃, —O—CF₃, —S—CF₃, —SH, —S—CH₃ and —S—C₂H₅; or represents a residue selected from the group consisting of phenyl, benzyl, phenethyl, pyridinyl, pyridazinyl, pyrimidinyl and pyrazinyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
R⁴, R⁵ and R⁶ each independently represent H; F; Cl; Br; I; —NO₂; —CN; —O—R²⁵; —S—R²⁶; or an alkyl residue selected from the group consisting of methyl, ethyl, n-propyl, isopropyl, n-butyl, 2-butyl, isobutyl and tert-butyl, each of which may be unsubstituted or substituted with 1, 2, 3, 4 or 5 substituents independently selected from the group consisting of F, Cl, Br, I, —NO₂ and —CN;
R⁷ and R⁸ each independently represent H; F; Cl; Br; I; —NO₂; —CN; —O—R²⁵; —S—R²⁶; or an alkyl residue selected from the group consisting of methyl, ethyl, n-propyl, isopropyl, n-butyl, 2-butyl, isobutyl and tert-butyl, each of which may be unsubstituted or substituted with 1, 2, 3, 4 or 5 substituents independently selected from the group consisting of F, Cl, Br, I, —NO₂ and —CN;
R⁹ represents H; —C(═O)—R¹⁵; —C(═O)—O—R¹⁶; —S(═O)₂—R²¹; an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, each of which may be unsubstituted or substituted with 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; a heteroalkyl residue selected from the group consisting of —CH₂—O—CH₃, —CH₂—O—C₂H₅, —CH₂—O—CH(CH₃)₂, —CH₂—O—C(CH₃)₃, —CH₂—CH₂—O—CH₃, —CH₂—CH₂—O—C₂H₅, —CH₂—CH₂—O—CH(CH₃)₂, —CH₂—CH₂—O—C(CH₃)₃, —CH₂—O—CH₂—O—C₂H₅ and —CH₂—O—CH₂—O—CH(CH₃)₂; a residue selected from the group consisting of cyclopropyl, cyclobutyl, cyclopentyl, cyclohexyl, cycloheptyl, cyclopentenyl and cyclohexenyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or a residue selected from the group consisting of pyridinyl, thiazolyl, pyridazinyl, pyrimidinyl, pyrazinyl, furanyl, —(CH₂)-furanyl, —(CH₂)—(CH₂)-furanyl, thienyl, phenyl, naphthyl, benzyl and phenethyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
R¹⁰ represents —C(═O)—R¹⁵; —C(═O)—O—R¹⁶; —C(═O)—NH—R¹⁷; —C(═S)—NH—R¹⁷; —C(═O)—NR¹⁸R¹⁹; —C(═S)—NR¹⁸R¹⁹; —S(═O)—R²⁰; —S(═O)₂—R²¹; or an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which is respectively substituted with optionally 1, 2, 3, 4 or 5 substituents independently selected from the group consisting of F, Cl, Br, I, —NO₂, —CN, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; and
if R¹ represents a residue selected from the group consisting of methyl, ethyl, n-propyl, isopropyl, n-butyl, isobutyl, 2-butyl, tert-butyl and cyclopropyl, then R¹⁰ can additionally represent H or for an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which can respectively be unsubstituted or substituted with 1, 2 or 3 substituents independently selected from the group consisting of —O—CH₃, —O—C₂H₅, —O—CH₂—C₂—CH₃, —O—CH(CH₃)₂ and —O—C(CH₃)₃; or
R⁹ and R¹⁰ together with the nitrogen atom to which they are bound represent a residue selected from the group consisting of imidazolidinyl, azepanyl, pyrrolyl, diazepanyl, isothioazolidinyl, (2,3)-dihydro-1H-isoindolyl, indolinyl, octahydro-1H-isoindolyl, (2,3)-dihydropyrrolyl, (2,5)-dihydropyrrolyl, 8-azaspiro[4.5]decyl, 8-azaspiro[4.5]decane-7,9-dionyl, isoindoline-1,3-dionyl, pyrrolidine-2,5-dionyl, morpholine-3,5-dionyl, 1H-pyrrole-2,5-dionyl, piperidine-2,6-dionyl, hexahydro-2H-isoindoline-1,3-dionyl, pyrrolidine-2-onyl and 1,1-dioxo-1λ⁶-isothiazolidinyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, thioxo, —O—CH₃, —O—C₂H₅, —O—C₃H₇, —NH—C(═O)—CF₃ and phenyl;
R¹¹, R¹², R¹³ and R¹⁴ each independently represent H; F; Cl; Br; I; —NO₂; —CN; —O—R²⁵; —S—R²⁶; or an alkyl residue selected from the group consisting of methyl, ethyl, n-propyl, isopropyl, n-butyl, 2-butyl, isobutyl and tert-butyl, each of which may be unsubstituted or substituted with 1, 2, 3, 4 or 5 substituents independently selected from the group consisting of F, Cl, Br, I, —NO₂, —CN, —OH, —SH and —NH₂;
R¹⁵ represents —C(═O)—O—R¹⁶; an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, each of which may be unsubstituted or substituted with 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; an alkenyl residue selected from the group consisting of ethenyl, 1-propenyl, 2-propenyl, 1-butenyl, 2-butenyl, 3-butenyl and —CH═C(CH₃)₂; a heteroalkyl residue selected from the group consisting of —CH₂—O—CH₃, —CH₂—O—C₂H₅, —CH₂—O—CH(CH₃)₂, —CH₂—O—C(CH₃)₃, —CH₂—CH₂—O—CH₃, —CH₂—CH₂—O—C₂H₅, —CH₂—CH₂—O—CH(CH₃)₂, —CH₂—CH₂—O—C(CH₃)₃, —CH₂—O—CH₂—O—C₂H₅ and —CH₂—O—CH₂—O—CH(CH₃)₂; a residue selected from the group consisting of cyclopropyl, cyclobutyl, cyclopentyl, cyclohexyl, cycloheptyl, cyclopentenyl and cyclohexenyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or represents a residue selected from the group consisting of phenyl, naphthyl, furanyl, thienyl, oxazolyl, oxadiazolyl, isoxazolyl, pyridinyl, pyridazinyl, pyrimidinyl, pyrazinyl, quinolinyl, isoquinolinyl and quinazolinyl, which can respectively be bonded by means of a —(CH₂)—, —(CH₂)—(CH₂)—, —(CH₂)—(CH₂)—(CH₂)—, —(CH═CH)— or —(CH₂)—O— group and/or is unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
R¹⁶, R¹⁷, R¹⁸, R¹⁹, R²⁰, R²¹, R²², R²³, R²⁴, R²⁵ and R²⁶ each independently represent an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, each of which may be unsubstituted or substituted with 1, 2, 3, 4 or 5 substituents independently selected from the group consisting of F, Cl, Br, I, —NO₂, —CN, —OH, —SH, —NH₂, —C(═O)—OH, —C(═O)—O—CH₃, —C(═O)—O—C₂H₅ and —C(═O)—O—C(CH₃)₃; a residue selected from the group consisting of cyclopropyl, cyclobutyl, cyclopentyl, cyclohexyl, cycloheptyl, cyclopentenyl and cyclohexenyl, which is respectively unsubstituted; or a residue selected from the group consisting of phenyl and benzyl, which is respectively unsubstituted or is substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NO₂, —CF₃, —O—CF₃, —S—CF₃, —SH, —C(═O)—O—CH₃ and —C(═O)—O—C₂H₅.

17. A compound as claimed in claim 1, wherein T, U, V and optionally W together with two carbon atoms form a ring selected from the group consisting of: 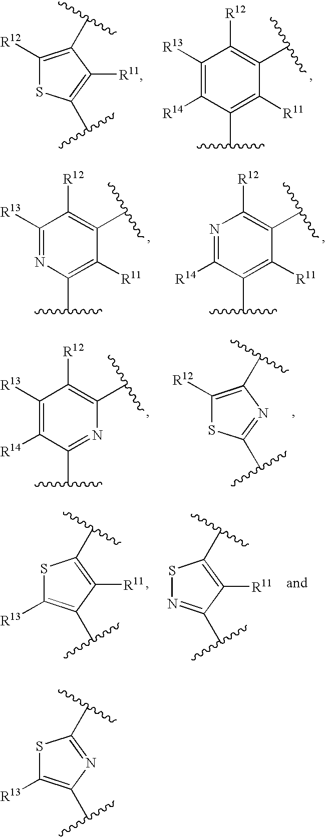
R¹ represents —C(═O)—R¹⁵; an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, each of which may be unsubstituted or substituted with 1, 2, 3, 4 or 5 substituents independently selected from the group consisting of F, Cl, Br, I, —NO₂, —CN, —OH, —OCH₃, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; a residue selected from the group consisting of cyclopropyl, cyclobutyl, cyclopentyl, cyclohexyl, cycloheptyl, cyclopentenyl and cyclohexenyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or represents a residue selected from the group consisting of phenyl, naphthyl, benzyl and phenethyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
R² represents H; —C(═O)—R¹⁵; or an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, each of which may be unsubstituted or substituted with 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂Hs), —C(═O)—OH, —C(═O)—O—CH₃, —C(═O)—O—C₂H₅, —C(═O)—O—C(CH₃)₃ and —NH—C(═O)—O—C(CH₃)₃; or
R¹ and R² together with the nitrogen atom to which they are bound represent a residue selected from the group consisting of piperazinyl, piperidinyl, morpholinyl, thiomorpholinyl, pyrrolidinyl, azepanyl and diazepanyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —NO₂, —CF₃, —O—CF₃, —S—CF₃, —SH, —S—CH₃ and —S—C₂H₅;
R³ represents H; F; Cl; Br; I; —NO₂; —CN; —NH₂; —OH; —SH; —NH—R²²; —NR²³R²⁴; —O—R²⁵; —S—R²⁶; an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, each of which may be unsubstituted or substituted with 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃ and —C(═O)—O—C₂H₅; a residue selected from the group consisting of morpholinyl, piperazinyl, piperidinyl, pyrrolidinyl, azepanyl, diazepanyl, thiomorpholinyl, cyclopropyl, cyclobutyl, cyclopentyl, cyclohexyl, cycloheptyl, cyclopentenyl and cyclohexenyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —NO₂, —CF₃, —O—CF₃, —S—CF₃, —SH, —S—CH₃ and —S—C₂H₅; or represents a residue selected from the group consisting of phenyl, benzyl, phenethyl, pyridinyl, pyridazinyl, pyrimidinyl and pyrazinyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
R⁴, R⁵ and R⁶ each independently represent H; F; Cl; Br; I; —NO₂; —CN; —O—R²⁵; —S—R²⁶; or an alkyl residue selected from the group consisting of methyl, ethyl, n-propyl, isopropyl, n-butyl, 2-butyl, isobutyl and tert-butyl, each of which may be unsubstituted or substituted with 1, 2, 3, 4 or 5 substituents independently selected from the group consisting of F, Cl, Br, I, —NO₂ and —CN;
R⁷ and R⁸ each independently represent H; F; Cl; Br; I; —NO₂; —CN; —O—R²⁵; —S—R²⁶; or an alkyl residue selected from the group consisting of methyl, ethyl, n-propyl, isopropyl, n-butyl, 2-butyl, isobutyl and tert-butyl, each of which may be unsubstituted or substituted with 1, 2, 3, 4 or 5 substituents independently selected from the group consisting of F, Cl, Br, I, —NO₂ and —CN;
R⁹ represents H; —C(═O)—R¹⁵; —C(═O)—O—R¹⁶; —S(═O)₂—R²¹; an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, each of which may be unsubstituted or substituted with 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; a —CH₂—CH═CH₂ or a —CH═CH—CH₃ residue, for a heteroalkyl residue selected from the group consisting of —CH₂—O—CH₃, —CH₂—O—C₂H₅, —CH₂—O—CH(CH₃)₂, —CH₂—O—C(CH₃)₃, —CH₂—CH₂—O—CH₃, —CH₂—CH₂—O—C₂H₅, —CH₂—CH₂—O—CH(CH₃)₂, —CH₂—CH₂—O—C(CH₃)₃, —CH₂—O—CH₂—O—C₂H₅ and —CH₂—O—CH₂—O—CH(CH₃)₂; a residue selected from the group consisting of cyclopropyl, cyclobutyl, cyclopentyl, cyclohexyl, cycloheptyl, cyclopentenyl and cyclohexenyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or a residue selected from the group phenyl, benzyl, phenethyl, pyridinyl, —(CH₂)-pyridinyl, thiazolyl, pyridazinyl, pyrimidinyl, pyrazinyl, furanyl, —(CH₂)-furanyl, —(CH₂)—(CH₂)-furanyl, thienyl and naphthyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
R¹⁰ represents —C(═O)—R¹⁵; —C(═O)—O—R¹⁶; —C(═O)—NH—R¹⁷; —C(═S)—NH—R¹⁷; —C(═O)—NR¹⁸R¹⁹; —C(═S)—NR¹⁸R¹⁹; —S(═O)—R²⁰; —S(═O)₂—R²¹; thiazolyl, or for an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which is respectively substituted with 1, 2, 3, 4 or 5 substituents independently selected from the group consisting of F, Cl, Br, I, —NO₂, —CN, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; and
if R¹ represents a residue selected from the group consisting of methyl, ethyl, n-propyl, isopropyl, n-butyl, isobutyl, 2-butyl, tert-butyl and cyclopropyl, then R¹⁰ can additionally represent H or for an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which can respectively be unsubstituted or substituted with 1, 2 or 3 substituents independently selected from the group consisting of —O—CH₃, —O—C₂H₅, —O—CH₂—C₂—CH₃, —O—CH(CH₃)₂ and —O—C(CH₃)₃; or
R⁹ and R¹⁰ together with the nitrogen atom to which they are bound represent a residue selected from the group consisting of 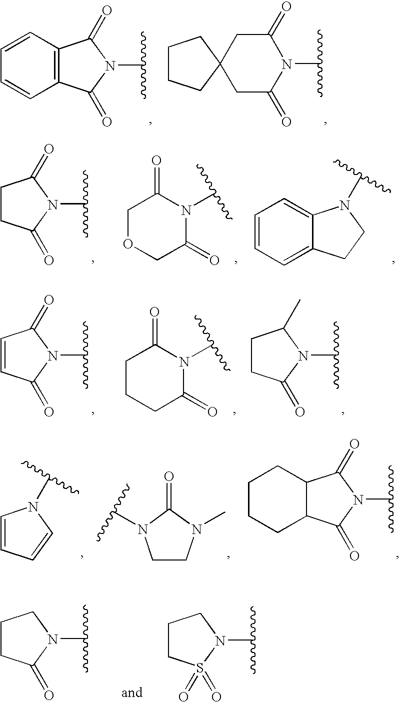
each of which may be unsubstituted or substituted with 1, 2 or 3 substituents independently selected from the group consisting of methyl, ethyl, n-propyl, isopropyl, n-butyl, 2-butyl, isobutyl, tert-butyl, —NH—C(═O)—CF₃ and phenyl;
R¹¹, R¹², R¹³ and R¹⁴ each independently represent H; F; Cl; Br; I; —NO₂; —CN; —O—R²⁵; —S—R²⁶; or an alkyl residue selected from the group consisting of methyl, ethyl, n-propyl, isopropyl, n-butyl, 2-butyl, isobutyl and tert-butyl, each of which may be unsubstituted or substituted with 1, 2, 3, 4 or 5 substituents independently selected from the group consisting of F, Cl, Br, I, —NO₂, —CN, —OH, —SH and —NH₂;
R¹⁵ represents —C(═O)—O—R¹⁶; an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, each of which may be unsubstituted or substituted with 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(—O)—O—C(CH₃)₃ and —NH—C(═O)—O—C(CH₃)₃; an alkenyl residue selected from the group consisting of ethenyl, 1-propenyl, 2-propenyl, 1-butenyl, 2-butenyl, 3-butenyl and —CH═C(CH₃)₂; a heteroalkyl residue selected from the group consisting of —CH₂—O—CH₃, —CH₂—O—C₂H₅, —CH₂—O—CH(CH₃)₂, —CH₂—O—C(CH₃)₃, —CH₂—CH₂—O—CH₃, —CH₂—CH₂—O—C₂H₅, —CH₂—CH₂—O—CH(CH₃)₂, —CH₂—CH₂—O—C(CH₃)₃, —CH₂—O—CH₂—O—C₂H₅ and —CH₂—O—CH₂—O—CH(CH₃)₂; a residue selected from the group consisting of piperidinyl, cyclopropyl, cyclobutyl, cyclopentyl, cyclohexyl, cycloheptyl, cyclopentenyl and cyclohexenyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NH—CO—(CH₂)₃—CH₃, —NO₂, —CF₃, —O—CF₃, —S—CF₃, —SH, —S—CH₃ and —S—C₂H₅; or represents a residue selected from the group consisting of phenyl, naphthyl, furanyl, thienyl, oxazolyl, oxadiazolyl, isoxazolyl, pyridinyl, pyridazinyl, pyrimidinyl, pyrazinyl, pyrazolyl, isoxazolyl, thiazolyl, quinolinyl, isoquinolinyl and quinazolinyl, which can respectively be bonded by means of a —(CH₂)—, —(CH₂)—(CH₂)—, —(CH₂)—(CH₂)—(CH₂)—, —(CH═CH)— or —(CH₂)—O— group and/or is unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅; and
R¹⁶, R¹⁷, R¹⁸, R¹⁹, R²⁰, R²¹, R²², R²³, R²⁴, R²⁵ and R²⁶ each independently represent an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, each of which may be unsubstituted or substituted with 1, 2, 3, 4 or 5 substituents independently selected from the group consisting of F, Cl, Br, I, —NO₂, —CN, —OH, —SH, —NH₂, —C(═O)—OH, —C(═O)—O—CH₃, —C(═O)—O—C₂H₅ and —C(═O)—O—C(CH₃)₃; a residue selected from the group consisting of cyclopropyl, cyclobutyl, cyclopentyl, cyclohexyl, cycloheptyl, cyclopentenyl and cyclohexenyl, which is respectively unsubstituted; or a residue selected from the group consisting of furanyl, thienyl, quinolinyl, isoquinolinyl, phenyl and benzyl, which is respectively unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NO₂, —CF₃, —O—CF₃, —S—CF₃, —SH, —C(═O)—O—CH₃ and —C(═O)—O—C₂H₅.

18. A compound as claimed in claim 1, wherein T, U, V and optionally W together with two carbon atoms form a ring selected from the group consisting of: 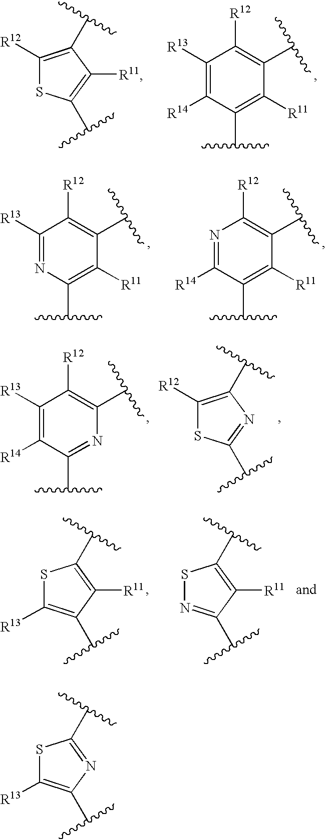
R¹ represents an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, each of which may be unsubstituted or substituted with 1, 2, 3, 4 or 5 substituents independently selected from the group consisting of F, Cl, Br, I, —NO₂, —CN, —OH, —OCH₃, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; a residue selected from the group consisting of cyclopropyl, cyclobutyl, cyclopentyl, cyclohexyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or represents a residue selected from the group consisting of phenyl, benzyl and phenethyl, which is respectively unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅ and —CF₃;
R² represents H or for an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, each of which may be unsubstituted or substituted with 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; or
R¹ and R² together with the nitrogen atom to which they are bound represent a residue selected from the group consisting of piperazinyl, piperidinyl, morpholinyl, thiomorpholinyl, pyrrolidinyl, azepanyl and diazepanyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —NO₂, —CF₃, —O—CF₃, —S—CF₃, —SH, —S—CH₃ and —S—C₂H₅;
R³ represents H; F; Cl; Br; I; —CN; —O—R²⁵; an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, each of which may be unsubstituted or substituted with 1, 2, 3, 4 or 5 substituents independently selected from the group consisting of F, Cl, Br, I, —CN and —OH;
R⁴, R⁵ and R⁶ each independently represent H; F; Cl; Br; I; —CN; —O—R²⁵; or an alkyl residue selected from the group consisting of methyl, ethyl, n-propyl, isopropyl, n-butyl, 2-butyl, isobutyl and tert-butyl, each of which may be unsubstituted or substituted with 1, 2, 3, 4 or 5 substituents independently selected from the group consisting of F, Cl, Br, I, —NO₂ and —CN;
R⁷ and R⁸ each represent H;
R⁹ represents H; —C(═O)—CH₃; —C(═O)—CH₂—CH₃; —C(═O)—O—R¹⁶; —S(═O)₂—R²¹; an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, each of which may be unsubstituted or substituted with 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; a —CH₂—CH═CH₂ or a —CH═CH—CH₃ residue, for a residue selected from the group consisting of cyclopropyl, cyclobutyl, cyclopentyl, cyclohexyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl and —CF₃; or a residue selected from the group phenyl, pyridinyl, —(CH₂)-pyridinyl, thiazolyl, pyridazinyl, pyrimidinyl, pyrazinyl, furanyl, —(CH₂)-furanyl, —(CH₂)—(CH₂)-furanyl, thienyl, phenyl, naphthyl, benzyl and phenethyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH and —CF₃;
R¹⁰ represents —C(═O)—R¹⁵; —C(═O)—O—R¹⁶; —S(═O)—R²⁰; thiazolyl, or for an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which is respectively substituted with 1, 2, 3, 4 or 5 substituents independently selected from the group consisting of F, Cl, Br, I, —NO₂, —CN, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; and
if R¹ represents a residue selected from the group consisting of methyl, ethyl, n-propyl, isopropyl, n-butyl, isobutyl, 2-butyl, tert-butyl and cyclopropyl, then R¹⁰ can additionally represent H or for an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, which can respectively be unsubstituted or substituted with 1, 2 or 3 substituents independently selected from the group consisting of —O—CH₃, —O—C₂H₅, —O—CH₂—C₂—CH₃, —O—CH(CH₃)₂ and —O—C(CH₃)₃; or
R⁹ and R¹⁰ together with the nitrogen atom to which they are bound represent a residue selected from the group consisting of 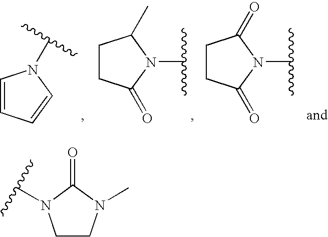
which is respectively unsubstituted or optionally substituted with 1, 2 or 3 substituents independently selected from the group consisting of methyl, ethyl, n-propyl, isopropyl, n-butyl, 2-butyl, isobutyl, tert-butyl, —NH—C(═O)—CF₃ and phenyl;
R¹¹, R¹², R¹³ and R¹⁴ each independently represent H; F; Cl; Br; I; —CN; —O—R²⁵; or an alkyl residue selected from the group consisting of methyl, ethyl, n-propyl, isopropyl, n-butyl, 2-butyl, isobutyl and tert-butyl, each of which may be unsubstituted or substituted with 1, 2, 3, 4 or 5 substituents independently selected from the group consisting of F, Cl, Br, I, —NO₂, —CN, —OH, —SH and —NH₂;
R¹⁵ represents —C(═O)—O—R¹⁶; an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, each of which may be unsubstituted or substituted with 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; an alkenyl residue selected from the group consisting of ethenyl, 1-propenyl, 2-propenyl, 1-butenyl, 2-butenyl, 3-butenyl and —CH═C(CH₃)₂; a heteroalkyl residue selected from the group consisting of —CH₂—O—CH₃, —CH₂—O—C₂H₅, —CH₂—O—CH(CH₃)₂, —CH₂—O—C(CH₃)₃, —CH₂—CH₂—O—CH₃, —CH₂—CH₂—O—C₂H₅, —CH₂—CH₂—O—CH(CH₃)₂, —CH₂—CH₂—O—C(CH₃)₃, —CH₂—O—CH₂—O—C₂H₅ and —CH₂—O—CH₂—O—CH(CH₃)₂; a residue selected from the group consisting of piperidinyl, cyclopropyl, cyclobutyl, cyclopentyl, cyclohexyl, cycloheptyl, cyclopentenyl and cyclohexenyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NH—CO—(CH₂)₃—CH₃, —NO₂, —CF₃, —O—CF₃, —S—CF₃, —SH, —S—CH₃ and —S—C₂H₅; or represents a residue selected from the group consisting of phenyl, naphthyl, furanyl, thienyl, oxazolyl, oxadiazolyl, isoxazolyl, pyridinyl, pyridazinyl, pyrimidinyl, pyrazinyl, pyrazolyl, isoxazolyl, thiazolyl, quinolinyl, isoquinolinyl and quinazolinyl, which can be respectively bonded by means of a —(CH₂)—, —(CH₂)—(CH₂)—, —(CH₂)—(CH₂)—(CH₂)—, —(CH═CH)— or —(CH₂)—O— group and/or is unsubstituted or substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
R¹⁶, R²⁰, R²¹ and R²⁵ each independently represent an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, each of which may be unsubstituted or substituted with 1, 2, 3, 4 or 5 substituents independently selected from the group consisting of F, Cl, Br, I, —NO₂, —CN, —OH, —SH, —NH₂, —C(═O)—OH, —C(═O)—O—CH₃, —C(═O)—O—C₂H₅ and —C(═O)—O—C(CH₃)₃; a residue selected from the group consisting of cyclopropyl, cyclobutyl, cyclopentyl, cyclohexyl, cycloheptyl, cyclopentenyl and cyclohexenyl, which is respectively unsubstituted; or a residue selected from the group consisting of furanyl, thienyl, quinolinyl, isoquinolinyl, phenyl and benzyl, which is respectively unsubstituted or is substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NO₂, —CF₃, —O—CF₃, —S—CF₃, —SH, —C(═O)—O—CH₃ and —C(═O)—O—C₂H₅.

19. A compound as claimed in claim 1, wherein T, U, V and optionally W together with two carbon atoms form a ring selected from the group consisting of: 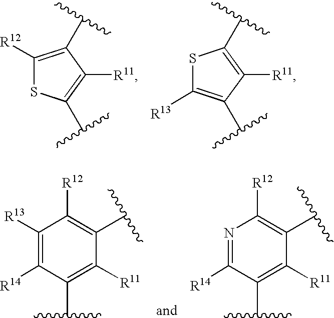
R¹ represents a residue selected from the group consisting of methyl, ethyl, n-propyl, isopropyl, n-butyl, 2-butyl, isobutyl, tert-butyl, n-pentyl, sec-pentyl, neo-pentyl and n-hexyl, —(CH₂)—C(═O)—OH and —(CH₂)—(CH₂)—C(═O)—OH; a residue selected from the group consisting of cyclopropyl, cyclobutyl, cyclopentyl, cyclohexyl, cycloheptyl, cyclopentenyl and cyclohexenyl; or a residue selected from the group consisting of phenyl, benzyl and phenethyl;
R² represents H; —C(═O)—CH₃; —C(═O)—C₂H₅; or an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl; or
R¹ and R² together with the nitrogen atom to which they are bound represent a residue selected from the group consisting of piperazinyl, piperidinyl, morpholinyl, thiomorpholinyl, pyrrolidinyl, azepanyl and diazepanyl;
R³ represents H; F; Cl; Br; —NH₂; —NH—R²²; —NR²³R²⁴; an alkyl residue selected from the group consisting of methyl, ethyl, n-propyl, isopropyl, n-butyl, 2-butyl, isobutyl, tert-butyl, n-pentyl, sec-pentyl, neo-pentyl, n-hexyl, —CF₃, —CHF₂, —CH₂F and —CF₂—CF₃; a residue selected from the group consisting of morpholinyl, piperazinyl, piperidinyl, pyrrolidinyl, azepanyl, diazepanyl and thiomorpholinyl; or a residue selected from the group consisting of phenyl, benzyl, phenethyl and pyridinyl;
R⁴, R⁵ and R⁶ each independently represent H, F, Cl, —CF₃, methyl, ethyl, —CN or —O—CH₃;
R⁷ and R⁸ each independently represent H or an alkyl residue selected from the group consisting of methyl, ethyl, n-propyl, isopropyl, n-butyl, 2-butyl, isobutyl and tert-butyl;
R⁹ represents H; —C(═O)—O—R¹⁶; an alkyl residue selected from the group consisting of methyl, ethyl, n-propyl, isopropyl, n-butyl, 2-butyl, isobutyl, tert-butyl, n-pentyl, sec-pentyl, neo-pentyl, n-hexyl, —(CH₂)—(CH₂)—NH—C(═O)—O—C(CH₃)₃, —(CH₂)—NH—C(═O)—O—C(CH₃)₃, —(CH₂)—CN, —(CH₂)—(CH₂)—CN, —(CH₂)—Cl, —(CH₂)—(CH₂)—Cl, —(CH₂)—NH₂, —(CH₂)—(CH₂)—NH₂, —(CH₂)—C(═O)—O—CH₃, —(CH₂)—(CH₂)—C(═O)—O—CH₃ and —(CH₂)—C(═O)—O—C₂H₅; a heteroalkyl residue selected from the group consisting of —CH₂—O—CH₃, —CH₂—O—C₂H₅, —CH₂—O—CH(CH₃)₂, —CH₂—O—C(CH₃)₃, —CH₂—CH₂—O—CH₃ and —CH₂—CH₂—O—C₂H₅; a residue selected from the group consisting of cyclopropyl, cyclobutyl, cyclopentyl, cyclohexyl, cycloheptyl, cyclopentenyl and cyclohexenyl; or a residue selected from the group consisting of pyridinyl, thiazolyl, —(CH₂)-furanyl, —(CH₂)—(CH₂)— furanyl, thienyl, phenyl, benzyl and phenethyl, each of which may be unsubstituted or is substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —O—CH₃, —O—C₂H₅ and —O—C₃H₇;
R¹⁰ represents —C(═O)—R¹⁵; —C(═O)—NH—R¹⁷; —C(═S)—NH—R¹⁷; —C(═O)—NR¹⁸R¹⁹; —C(═S)—NR¹⁸R¹⁹ or —S(═O)₂—R²¹; and
if R¹ represents a residue selected from the group consisting of methyl, ethyl, n-propyl, isopropyl, n-butyl, isobutyl, 2-butyl, tert-butyl and cyclopropyl, then R¹⁰ additionally may represent H or an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, each of which may be unsubstituted or substituted with 1, 2 or 3 substituents independently selected from the group consisting of —O—CH₃, —O—C₂H₅, —O—CH₂—C₂—CH₃, —O—CH(CH₃)₂ and —O—C(CH₃)₃; or
R⁹ and R¹⁰ together with the nitrogen atom to which they are bound represent a residue selected from the group consisting of 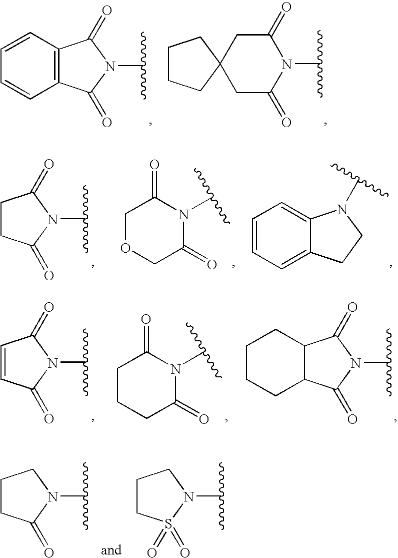
which may be unsubstituted or substituted with 1, 2 or 3 substituents independently selected from the group consisting of methyl, ethyl, n-propyl, isopropyl, n-butyl, 2-butyl, isobutyl, tert-butyl, —NH—C(═O)—CF₃ and phenyl;
R¹¹ represents H; F; Cl; Br; —O—CH₃; —O—C₂H₅; —O—CH(CH₃)₂; —O—C(CH₃)₃; methyl; ethyl; n-propyl or isopropyl;
R¹² represents H; F; Cl; Br; —O—CH₃; —O—C₂H₅; —O—CH(CH₃)₂; —O—C(CH₃)₃; methyl; ethyl; n-propyl or isopropyl;
R¹³ represents H; F; Cl; Br; —O—CH₃; —O—C₂H₅; —O—CH(CH₃)₂; —O—C(CH₃)₃; methyl; ethyl; n-propyl or isopropyl;
R¹⁴ represents H; F; Cl; Br; —O—CH₃; —O—C₂H₅; —O—CH(CH₃)₂; —O—C(CH₃)₃; methyl; ethyl; n-propyl or isopropyl;
R¹⁵ represents —C(═O)—O—R¹⁶; an alkyl group from the group consisting of methyl, ethyl, n-propyl, isopropyl, n-butyl, 2-butyl, isobutyl, tert-butyl, n-pentyl, sec-pentyl, neo-pentyl, n-hexyl, —CF₃, —(CH₂)—F, —CHF₂, —(CH₂)—Cl, —(CH₂)—CF₃, —CF₂—CF₃, —(CH₂)—CN, —(CH₂)—N(CH₃)₂, —(CH₂)—(CH₂)—CN, —(CH₂)—(CH₂)—N(CH₃)₂, —(CH₂)—C(═O)—O—CH₃, —(CH₂)—C(═O)—OH, —(CH₂)—(CH₂)—C(═O)—O—CH₃ and —(CH₂)—(CH₂)—C(═O)—OH; an alkenyl residue selected from the group consisting of ethenyl, 1-propenyl, 2-propenyl, 1-butenyl, 2-butenyl, 3-butenyl and —CH═C(CH₃)₂; a heteroalkyl residue selected from the group consisting of —CH₂—O—CH₃, —CH₂—O—C₂H₅, —CH₂—O—CH(CH₃)₂, —CH₂—O—C(CH₃)₃, —CH₂—CH₂—O—CH₃ and —CH₂—CH₂—O—C₂H₅; a residue selected from the group consisting of cyclopropyl, cyclobutyl, cyclopentyl, cyclohexyl, cycloheptyl, cyclopentenyl and cyclohexenyl; a residue selected from the group consisting of phenyl and pyridinyl, each of which optionally may be bonded via a —(CH₂)—, —(CH₂)—(CH₂)—, —(CH₂)—(CH₂)—(CH₂)—, —(CH═CH)— or —(CH₂)—O— group and which may be unsubstituted or substituted with 1, 2, 3, 4 or 5 substituents independently selected from the group consisting of F, Cl, Br, I, —C(═O)—OH, —C(═O)—O—CH₃ and —C(═O)—O—C₂H₅; or a residue selected from the group consisting of phenyl, furanyl, thienyl, oxazolyl, isoxazolyl, pyridinyl, pyrazinyl, quinolinyl and isoquinolinyl, each of which may optionally be substituted 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NO₂, —CF₃, —O—CF₃ and —S—CF₃;
R¹⁶ represents an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl;
R¹⁷, R¹⁸ and R¹⁹ each independently represent an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl;
R²¹ represents an alkyl residue selected from the group consisting of methyl, ethyl, n-propyl, isopropyl, n-butyl, 2-butyl, isobutyl, tert-butyl, n-pentyl, sec-pentyl, neo-pentyl, n-hexyl, —CF₃, —CHF₂, —CH₂F, —CF₂—CF₃, —(CH₂)—CF₃, —(CH₂)—Cl, —(CH₂)—(CH₂)—Cl, —(CH₂)—(CH₂)—(CH₂)—Cl and —(CH₂)—(CH₂)—(CH₂)—(CH₂)—Cl; or a residue selected from the group consisting of phenyl and benzyl, which is respectively unsubstituted or substituted with 1, 2, 3, 4 or 5 substituents independently selected from the group consisting of F, Cl, Br and —CF₃; and
R²², R²³ and R²⁴ each independently represent an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl; a residue selected from the group consisting of cyclopropyl, cyclobutyl, cyclopentyl, cyclohexyl, cycloheptyl, cyclopentenyl and cyclohexenyl; or a residue selected from the group consisting of phenyl and benzyl.

20. A compound as claimed in claim 1, corresponding to formula Ia 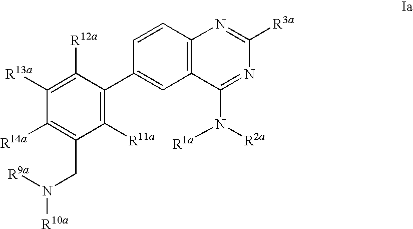
wherein,
R^{1a} represents —C(═O)—R^{15a}; an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, each of which may be unsubstituted or substituted with 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; a residue selected from the group consisting of cyclopropyl, cyclobutyl, cyclopentyl, cyclohexyl, cycloheptyl, cyclopentenyl and cyclohexenyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or a residue selected from the group consisting of phenyl, naphthyl, benzyl and phenethyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
R^{2a} represents H; —C(═O)—R^{15a}; or represents an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, each of which may be unsubstituted or substituted with 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; or
R^{1a} and R^{2a} together with the nitrogen atom to which they are bound represent a residue selected from the group consisting of piperazinyl, piperidinyl, morpholinyl, thiomorpholinyl, pyrrolidinyl, azepanyl and diazepanyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —NO₂, —CF₃, —O—CF₃, —S—CF₃, —SH, —S—CH₃ and —S—C₂H₅;
R^{3a} represents H; F; Cl; Br; I; —NO₂; —CN; —NH₂; —OH; —SH; —NH_R^{22a}; —NR^{23a}R^{24a}; —O—R^{25a}; —S—R^{26a}; an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, each of which may be unsubstituted or substituted with 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃ and —C(═O)—O—C₂H₅; a residue selected from the group consisting of morpholinyl, piperazinyl, piperidinyl, pyrrolidinyl, azepanyl, diazepanyl, thiomorpholinyl, cyclopropyl, cyclobutyl, cyclopentyl, cyclohexyl, cycloheptyl, cyclopentenyl and cyclohexenyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —NO₂, —CF₃, —O—CF₃, —S—CF₃, —SH, —S—CH₃ and —S—C₂H₅; or a residue selected from the group consisting of phenyl, benzyl, phenethyl, pyridinyl, pyridazinyl, pyrimidinyl and pyrazinyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
R^{9a} represents H; —C(═O)—R^{15a}; —C(═O)—O—R^{16a}; —S(═O)₂—R^{21a}; an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, each of which may be unsubstituted or substituted with 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; a heteroalkyl residue selected from the group consisting of —CH₂—O—CH₃, —CH₂—O—C₂H₅, —CH₂—O—CH(CH₃)₂, —CH₂—O—C(CH₃)₃, —CH₂—CH₂—O—CH₃, —CH₂—CH₂—O—C₂H₅, —CH₂—CH₂—O—CH(CH₃)₂, —CH₂—CH₂—O—C(CH₃)₃, —CH₂—O—CH₂—O—C₂H₅ and —CH₂—O—CH₂—O—CH(CH₃)₂; a residue selected from the group consisting of cyclopropyl, cyclobutyl, cyclopentyl, cyclohexyl, cycloheptyl, cyclopentenyl and cyclohexenyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, thioxo, oxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or a residue selected from the group consisting of pyridinyl, thiazolyl, pyridazinyl, pyrimidinyl, pyrazinyl, furanyl, —(CH₂)-furanyl, —(CH₂)—(CH₂)-furanyl, thienyl, phenyl, naphthyl, benzyl and phenethyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
R^{10a} represents —C(═O)—R^{15a}; —C(═O)—O—R^{16a}; —C(═O)—NH—R^{17a}; —C(═O)—NR^{18a}R^{19a}; —S(═O)—R^{20a}; —S(═O)₂—R^{21a}; or an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, each of which optionally may be substituted with 1, 2, 3, 4 or 5 substituents independently selected from the group consisting of F, Cl, Br, I, —NO₂, —CN, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; and
if R^{1a} represents a residue selected from the group consisting of methyl, ethyl, n-propyl, isopropyl, n-butyl, isobutyl, 2-butyl, tert-butyl and cyclopropyl stent, then R^{10a} additionally may represent H or an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, each of which may be unsubstituted or substituted with 1, 2 or 3 substituents independently selected from the group consisting of —O—CH₃, —O—C₂H₅, —O—CH₂—C₂—CH₃, —O—CH(CH₃)₂ and —O—C(CH₃)₃; or
R^{9a} and R^{10a} together with the nitrogen atom to which they are bound represent a residue selected from the group consisting of imidazolidinyl, azepanyl, pyrrolyl, diazepanyl, isothioazolidinyl, (2,3)-dihydro-1H-isoindolyl, indolinyl, octahydro-1H-isoindolyl, (2,3)-dihydropyrrolyl, (2,5)-dihydropyrrolyl, 8-azaspiro[4.5]decyl, 8-azaspiro[4.5]-decane-7,9-dionyl, isoindoline-1,3-dionyl, pyrrolidine-2,5-dionyl, morpholine-3,5-dionyl, 1H-pyrrole-2,5-dionyl, piperidine-2,6-dionyl, hexahydro-2H-isoindoline-1,3-dionyl, pyrrolidine-2-onyl and 1,1-dioxo-1λ⁶-isothiazolidinyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, thioxo, —O—CH₃, —O—C₂H₅, —O—C₃H₇, —NH—C(═O)—CF₃ and phenyl; wherein the phenyl residue may be unsubstituted or substituted with 1, 2, 3, 4 or 5 substituents independently selected from the group consisting of F, Cl, Br, I, —CN, —CF₃, —O—CF₃, —O—CH₃, —O—C₂H₅, —O—C₃H₇, methyl, ethyl, n-propyl, isopropyl, n-butyl, 2-butyl, isobutyl and tert-butyl;
R^{11a}, R^{12a}, R^{13a} and R^{14a} each independently represent H; F; Cl; Br; I; —NO₂; —CN; —O—R²⁵; —S—R²⁶; or an alkyl residue selected from the group consisting of methyl, ethyl, n-propyl, isopropyl, n-butyl, 2-butyl, isobutyl and tert-butyl, each of which may be unsubstituted or substituted with 1, 2, 3, 4 or 5 substituents independently selected from the group consisting of F, Cl, Br, I, —NO₂, —CN, —OH, —SH and —NH₂;
R^{15a} represents —C(═O)—O—R^{16a}; an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, each of which may be unsubstituted or substituted with 1, 2, 3, 4 or 5 substituents independently selected from the group consisting of F, Cl, Br, I, —NO₂, —CN, —OH, —SH, —NH₂, —N(CH₃)₂, —N(C₂H₅)₂, —N(CH₃)(C₂H₅), —C(═O)—OH, —C(═O)—O—CH₃, —C(═O)—O—C₂H₅, —C(═O)—O—C(CH₃)₃ and —NH—C(═O)—O—C(CH₃)₃; an alkenyl residue selected from the group consisting of ethenyl, 1-propenyl, 2-propenyl, 1-butenyl, 2-butenyl, 3-butenyl and —CH═C(CH₃)₂; a heteroalkyl residue selected from the group consisting of —CH₂—O—CH₃, —CH₂—O—C₂H₅, —CH₂—O—CH(CH₃)₂, —CH₂—O—C(CH₃)₃, —CH₂—CH₂—O—CH₃, —CH₂—CH₂—O—C₂H₅, —CH₂—CH₂—O—CH(CH₃)₂, —CH₂—CH₂—O—C(CH₃)₃, —CH₂—O—CH₂—O—C₂H₅ and —CH₂—O—CH₂—O—CH(CH₃)₂; a residue selected from the group consisting of cyclopropyl, cyclobutyl, cyclopentyl, cyclohexyl, cycloheptyl, cyclopentenyl and cyclohexenyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, oxo, thioxo, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —S—CH₃ and —S—C₂H₅; or a residue selected from the group consisting of phenyl, naphthyl, furanyl, thienyl, oxazolyl, oxadiazolyl, isoxazolyl, pyridinyl, pyridazinyl, pyrimidinyl, pyrazinyl, quinolinyl, isoquinolinyl and quinazolinyl, each of which optionally may be bonded via a —(CH₂)—, —(CH₂)—(CH₂)—, —(CH₂)—(CH₂)—(CH₂)—, —(CH═CH)— or —(CH₂)—O— group and which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NH₂, —N(CH₃)₂, —N(C₂H₅)₂, —NH—CH₃, —NH—C₂H₅, —NO₂, —CF₃, —O—CF₃, —S—CF₃, —SH, —NH—S(═O)₂—CH₃, —C(═O)—OH, —C(═O)—CH₃, —C(═O)—C₂H₅, —C(═O)—N(CH₃)₂, —C(═O)—NH—CH₃, —NH—C(═O)—CH₃, —NH—C(═O)—C₂H₅, —C(═O)—O—CH₃ and —C(═O)—O—C₂H₅;
R^{16a}, R^{17a}, R^{18a}, R^{19a}, R^{20a}, R^{21a}, R^{22a}, R^{23a}, R^{24a}, R^{25a} and R^{26a} each independently represent an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl, each of which may be unsubstituted or substituted with 1, 2, 3, 4 or 5 substituents independently selected from the group consisting of F, Cl, Br, I, —NO₂, —CN, —OH, —SH, —NH₂, —C(═O)—OH, —C(═O)—O—CH₃, —C(═O)—O—C₂H₅ and —C(═O)—O—C(CH₃)₃; an unsubstituted residue selected from the group consisting of cyclopropyl, cyclobutyl, cyclopentyl, cyclohexyl, cycloheptyl, cyclopentenyl and cyclohexenyl; or a residue selected from the group consisting of phenyl and benzyl, each of which may be unsubstituted or is substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NO₂, —CF₃, —O—CF₃, —S—CF₃, —SH, —C(═O)—O—CH₃ and —C(═O)—O—C₂H₅;
or a salt thereof.

21. A compound as claimed in claim 1, corresponding to formula Ib 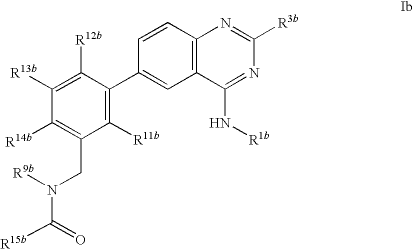
wherein
R^{1b} represents a residue selected from the group consisting of methyl, ethyl, n-propyl, isopropyl, n-butyl, 2-butyl, isobutyl, tert-butyl, n-pentyl, sec-pentyl, neo-pentyl, n-hexyl, —(CH₂)—C(═O)—OH and —(CH₂)—(CH₂)—C(═O)—OH; a residue selected from the group consisting of cyclopropyl, cyclobutyl, cyclopentyl, cyclohexyl, cycloheptyl, cyclopentenyl and cyclohexenyl; or a residue selected from the group consisting of phenyl, benzyl and phenethyl;
R^{3b} represents H; F; Cl; Br; —NH₂; —NH—R^{22b}; —NR^{23b}R^{24b}; an alkyl residue selected from the group consisting of methyl, ethyl, n-propyl, isopropyl, n-butyl, 2-butyl, isobutyl, tert-butyl, n-pentyl, sec-pentyl, neo-pentyl, n-hexyl, —CF₃, —CHF₂, —CH₂F and —CF₂—CF₃; a residue selected from the group consisting of morpholinyl, piperazinyl, piperidinyl, pyrrolidinyl, azepanyl, diazepanyl and thiomorpholinyl; or a residue selected from the group consisting of phenyl, benzyl, phenethyl and pyridinyl;
R^{9b} represents H; —C(═O)—O—R^{16b}; an alkyl residue selected from the group consisting of methyl, ethyl, n-propyl, isopropyl, n-butyl, 2-butyl, isobutyl, tert-butyl, n-pentyl, sec-pentyl, neo-pentyl, n-hexyl, —(CH₂)—(CH₂)—NH—C(═O)—O—C(CH₃)₃, —(CH₂)—NH—C(═O)—O—C(CH₃)₃, —(CH₂)—CN, —(CH₂)—(CH₂)—CN, —(CH₂)—Cl, —(CH₂)—(CH₂)—Cl, —(CH₂)—NH₂, —(CH₂)—(CH₂)—NH₂, —(CH₂)—C(═O)—O—CH₃, —(CH₂)—(CH₂)—C(═O)—O—CH₃ and —(CH₂)—C(═O)—O—C₂H₅; a heteroalkyl residue selected from the group consisting of —CH₂—O—CH₃, —CH₂—O—C₂H₅, —CH₂—O—CH(CH₃)₂, —CH₂—O—C(CH₃)₃, —CH₂—CH₂—O—CH₃ and —CH₂—CH₂—O—C₂H₅; a residue selected from the group consisting of cyclopropyl, cyclobutyl, cyclopentyl, cyclohexyl, cycloheptyl, cyclopentenyl and cyclohexenyl; or a residue selected from the group consisting of pyridinyl, thiazolyl, —(CH₂)-furanyl, —(CH₂)—(CH₂)-furanyl, thienyl, phenyl, benzyl and phenethyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —O—CH₃, —O—C₂H₅ and —O—C₃H₇;
R^{11b} represents H; F; Cl; Br; —O—CH₃; —O—C₂H₅; —O—CH(CH₃)₂; —O—C(CH₃)₃; methyl; ethyl; n-propyl or isopropyl;
R^{12b} represents H; F; Cl; Br; —O—CH₃; —O—C₂H₅; —O—CH(CH₃)₂; —O—C(CH₃)₃; methyl; ethyl; n-propyl or isopropyl;
R^{13b} represents H; F; Cl; Br; —O—CH₃; —O—C₂H₅; —O—CH(CH₃)₂; —O—C(CH₃)₃; methyl; ethyl; n-propyl or isopropyl;
R^{14b} represents H; F; Cl; Br; —O—CH₃; —O—C₂H₅; —O—CH(CH₃)₂; —O—C(CH₃)₃; methyl; ethyl; n-propyl or isopropyl;
R^{15b} represents —C(═O)—O—R^{16b}; an alkyl residue selected from the group consisting of methyl, ethyl, n-propyl, isopropyl, n-butyl, 2-butyl, isobutyl, tert-butyl, n-pentyl, sec-pentyl, neo-pentyl, n-hexyl, —CF₃, —(CH₂)—F, —CHF₂, —(CH₂)—Cl, —(CH₂)—CF₃, —CF₂—CF₃, —(CH₂)—CN, —(CH₂)—N(CH₃)₂, —(CH₂)—(CH₂)—CN, —(CH₂)—(CH₂)—N(CH₃)₂, —(CH₂)—C(═O)—O—CH₃, —(CH₂)—C(═O)—OH, —(CH₂)—(CH₂)—C(═O)—O—CH₃ and —(CH₂)—(CH₂)—C(═O)—OH; an alkenyl residue selected from the group consisting of ethenyl, 1-propenyl, 2-propenyl, 1-butenyl, 2-butenyl, 3-butenyl and —CH═C(CH₃)₂; a heteroalkyl residue selected from the group consisting of —CH₂—O—CH₃, —CH₂—O—C₂H₅, —CH₂—O—CH(CH₃)₂, —CH₂—O—C(CH₃)₃, —CH₂—CH₂—O—CH₃ and —CH₂—CH₂—O—C₂H₅; a residue selected from the group consisting of cyclopropyl, cyclobutyl, cyclopentyl, cyclohexyl, cycloheptyl, cyclopentenyl and cyclohexenyl; a residue selected from the group consisting of phenyl and pyridinyl, which is respectively bonded by means of a —(CH₂)—, —(CH₂)—(CH₂)—, —(CH₂)—(CH₂)—(CH₂)—, —(CH═CH)— or —(CH₂)—O— group and/or is unsubstituted or substituted with optionally 1, 2, 3, 4 or 5 substituents independently selected from the group consisting of F, Cl, Br, I, —C(═O)—OH, —C(═O)—O—CH₃ and —C(═O)—O—C₂H₅; or represents a residue selected from the group consisting of phenyl, furanyl, thienyl, oxazolyl, isoxazolyl, pyridinyl, pyrazinyl, quinolinyl and isoquinolinyl, which is respectively substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NO₂, —CF₃, —O—CF₃ and —S—CF₃;
R^{16b} represents an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl; and
R^{22b}, R^{23b} and R^{24b} each independently represent an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl; a residue selected from the group consisting of cyclopropyl, cyclobutyl, cyclopentyl, cyclohexyl, cycloheptyl, cyclopentenyl and cyclohexenyl; or a residue selected from the group consisting of phenyl and benzyl;
or a salt thereof.

22. A compound as claimed in claim 21, wherein:
R^{1b} represents a residue selected from the group consisting of methyl, ethyl, n-propyl, isopropyl, n-butyl, 2-butyl, isobutyl, tert-butyl, n-pentyl, sec-pentyl, neo-pentyl, n-hexyl, —(CH₂)—C(═O)—OH and —(CH₂)—(CH₂)—C(═O)—OH;
R^{3b} represents H;
R^{9b} represents H; —C(═O)—O—R^{16b}; an alkyl residue selected from the group consisting of methyl, ethyl, n-propyl, isopropyl, n-butyl, 2-butyl, isobutyl, tert-butyl, n-pentyl, sec-pentyl, neo-pentyl, n-hexyl, —(CH₂)—(CH₂)—NH—C(═O)—O—C(CH₃)₃, —(CH₂)—NH—C(═O)—O—C(CH₃)₃, —(CH₂)—CN, —(CH₂)—(CH₂)—CN, —(CH₂)—Cl, —(CH₂)—(CH₂)—Cl, —(CH₂)—NH₂, —(CH₂)—(CH₂)—NH₂, —(CH₂)—C(═O)—O—CH₃, —(CH₂)—(CH₂)—C(═O)—O—CH₃ and —(CH₂)—C(═O)—O—C₂H₅; a heteroalkyl residue selected from the group consisting of —CH₂—O—CH₃, —CH₂—O—C₂H₅, —CH₂—O—CH(CH₃)₂, —CH₂—O—C(CH₃)₃, —CH₂—CH₂—O—CH₃ and —CH₂—CH₂—O—C₂H₅; a residue selected from the group consisting of cyclopropyl, cyclobutyl, cyclopentyl, cyclohexyl, cycloheptyl, cyclopentenyl and cyclohexenyl; or a residue selected from the group consisting of pyridinyl, thiazolyl, —(CH₂)-furanyl, —(CH₂)—(CH₂)-furanyl, thienyl, phenyl, benzyl and phenethyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —O—CH₃, —O—C₂H₅ and —O—C₃H₇;
R^{11b} represents H;
R^{12b} represents H;
R^{13b} represents H;
R^{14b} represents H;
R^{15b} represents —C(═O)—O—R^{16b}; an alkyl residue selected from the group consisting of methyl, ethyl, n-propyl, isopropyl, n-butyl, 2-butyl, isobutyl, tert-butyl, n-pentyl, sec-pentyl, neo-pentyl, n-hexyl, —CF₃, —(CH₂)—F, —CHF₂, —(CH₂)—Cl, —(CH₂)—CF₃, —CF₂—CF₃, —(CH₂)—CN, —(CH₂)—N(CH₃)₂, —(CH₂)—(CH₂)—CN, —(CH₂)—(CH₂)—N(CH₃)₂, —(CH₂)—C(═O)—O—CH₃, —(CH₂)—C(═O)—OH, —(CH₂)—(CH₂)—C(═O)—O—CH₃ and —(CH₂)—(CH₂)—C(═O)—OH; an alkenyl residue selected from the group consisting of ethenyl, 1-propenyl, 2-propenyl, 1-butenyl, 2-butenyl, 3-butenyl and —CH═C(CH₃)₂; a heteroalkyl residue selected from the group consisting of —CH₂—O—CH₃, —CH₂—O—C₂H₅, —CH₂—O—CH(CH₃)₂, —CH₂—O—C(CH₃)₃, —CH₂—CH₂—O—CH₃ and —CH₂—CH₂—O—C₂H₅; a residue selected from the group consisting of cyclopropyl, cyclobutyl, cyclopentyl, cyclohexyl, cycloheptyl, cyclopentenyl and cyclohexenyl; a residue selected from the group consisting of phenyl and pyridinyl, which is respectively bonded by means of a —(CH₂)—, —(CH₂)—(CH₂)—, —(CH₂)—(CH₂)—(CH₂)—, —(CH═CH)— or —(CH₂)—O— group and/or is unsubstituted or substituted with optionally 1, 2, 3, 4 or 5 substituents independently selected from the group consisting of F, Cl, Br, I, —C(═O)—OH, —C(═O)—O—CH₃ and —C(═O)—O—C₂H₅; or represents a residue selected from the group consisting of phenyl, furanyl, thienyl, oxazolyl, isoxazolyl, pyridinyl, pyrazinyl, quinolinyl and isoquinolinyl, which is respectively substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NO₂, —CF₃, —O—CF₃ and —S—CF₃; and
R^{16b} represents an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl.

23. A compound as claimed in claim 1, corresponding to formula Ic 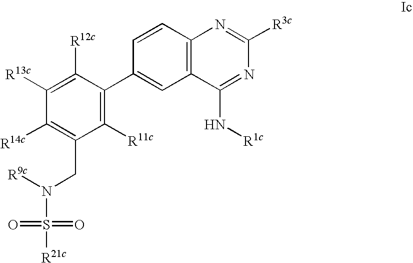
wherein
R^{1c} represents a residue selected from the group consisting of methyl, ethyl, n-propyl, isopropyl, n-butyl, 2-butyl, isobutyl, tert-butyl, n-pentyl, sec-pentyl, neo-pentyl, n-hexyl, —(CH₂)—C(═O)—OH and —(CH₂)—(CH₂)—C(═O)—OH; a residue selected from the group consisting of cyclopropyl, cyclobutyl, cyclopentyl, cyclohexyl, cycloheptyl, cyclopentenyl and cyclohexenyl; or a residue selected from the group consisting of phenyl, benzyl and phenethyl;
R^{3c} represents H; F; Cl; Br; —NH₂; —NH—R^{22c}; —NR^{23c}R^{24c}; an alkyl residue selected from the group consisting of methyl, ethyl, n-propyl, isopropyl, n-butyl, 2-butyl, isobutyl, tert-butyl, n-pentyl, sec-pentyl, neo-pentyl, n-hexyl, —CF₃, —CHF₂, —CH₂F and —CF₂—CF₃; a residue selected from the group consisting of morpholinyl, piperazinyl, piperidinyl, pyrrolidinyl, azepanyl, diazepanyl and thiomorpholinyl; or a residue selected from the group consisting of phenyl, benzyl, phenethyl and pyridinyl;
R^{9c} represents H; —C(═O)—O—R^{16c}; an alkyl residue selected from the group consisting of methyl, ethyl, n-propyl, isopropyl, n-butyl, 2-butyl, isobutyl, tert-butyl, n-pentyl, sec-pentyl, neo-pentyl, n-hexyl, —(CH₂)—(CH₂)—NH—C(═O)—O—C(CH₃)₃, —(CH₂)—NH—C(═O)—O—C(CH₃)₃, —(CH₂)—CN, —(CH₂)—(CH₂)—CN, —(CH₂)—Cl, —(CH₂)—(CH₂)—Cl, —(CH₂)—NH₂, —(CH₂)—(CH₂)—NH₂, —(CH₂)—C(═O)—O—CH₃, —(CH₂)—(CH₂)—C(═O)—O—CH₃ and —(CH₂)—C(═O)—O—C₂H₅; a heteroalkyl residue selected from the group consisting of —CH₂—O—CH₃, —CH₂—O—C₂H₅, —CH₂—O—CH(CH₃)₂, —CH₂—O—C(CH₃)₃, —CH₂—CH₂—O—CH₃ and —CH₂—CH₂—O—C₂H₅; a residue selected from the group consisting of cyclopropyl, cyclobutyl, cyclopentyl, cyclohexyl, cycloheptyl, cyclopentenyl and cyclohexenyl; or a residue selected from the group consisting of pyridinyl, thiazolyl, —(CH₂)-furanyl, —(CH₂)—(CH₂)-furanyl, thienyl, phenyl, benzyl and phenethyl, each of which may be unsubstituted or substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —O—CH₃, —O—C₂H₅ and —O—C₃H₇;
R^{11c} represents H; F; Cl; Br; —O—CH₃; —O—C₂H₅; —O—CH(CH₃)₂; —O—C(CH₃)₃; methyl; ethyl; n-propyl or isopropyl;
R^{12c} represents H; F; Cl; Br; —O—CH₃; —O—C₂H₅; —O—CH(CH₃)₂; —O—C(CH₃)₃; methyl; ethyl; n-propyl or isopropyl;
R^{13c} represents H; F; Cl; Br; —O—CH₃; —O—C₂H₅; —O—CH(CH₃)₂; —O—C(CH₃)₃; methyl; ethyl; n-propyl or isopropyl;
R^{14c} represents H; F; Cl; Br; —O—CH₃; —O—C₂H₅; —O—CH(CH₃)₂; —O—C(CH₃)₃; methyl; ethyl; n-propyl or isopropyl;
R^{16c} represents alkyl residue selected from the group consisting of methyl, ethyl, n-propyl, isopropyl, n-butyl, 2-butyl, isobutyl, tert-butyl, n-pentyl, sec-pentyl, neo-pentyl, n-hexyl;
R^{21c} represents an alkyl residue selected from the group consisting of methyl, ethyl, n-propyl, isopropyl, n-butyl, 2-butyl, isobutyl, tert-butyl, n-pentyl, sec-pentyl, neo-pentyl, n-hexyl, —CF₃, —(CH₂)—F, —CHF₂, —(CH₂)—Cl, —(CH₂)—CF₃, —CF₂—CF₃, —(CH₂)—CN, —(CH₂)—N(CH₃)₂, —(CH₂)—(CH₂)—CN, —(CH₂)—(CH₂)—N(CH₃)₂, —(CH₂)—C(═O)—O—CH₃, —(CH₂)—C(═O)—OH, —(CH₂)—(CH₂)—C(═O)—O—CH₃ and —(CH₂)—(CH₂)—C(═O)—OH; an unsubstituted residue selected from the group consisting of cyclopropyl, cyclobutyl, cyclopentyl, cyclohexyl, cycloheptyl, cyclopentenyl and cyclohexenyl; or a residue selected from the group consisting of phenyl and benzyl, each of which may be unsubstituted or is substituted with 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —OH, —O—CH₃, —O—C₂H₅, —O—C₃H₇, —NO₂, —CF₃, —O—CF₃, —S—CF₃, —SH, —C(═O)—O—CH₃ and —C(═O)—O—C₂H₅; and
R^{22c}, R^{23c} and R^{24c} each independently represent an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl; a residue selected from the group consisting of cyclopropyl, cyclobutyl, cyclopentyl, cyclohexyl, cycloheptyl, cyclopentenyl and cyclohexenyl; or a residue selected from the group consisting of phenyl and benzyl;
or a salt thereof.

24. A compound as claimed in claim 23, wherein:
R^{1c} represents a residue selected from the group consisting of methyl, ethyl, n-propyl, isopropyl, n-butyl, 2-butyl, isobutyl, tert-butyl, n-pentyl, sec-pentyl, neo-pentyl, n-hexyl, —(CH₂)—C(═O)—OH and —(CH₂)—(CH₂)—C(═O)—OH;
R^{3c} represents H;
R^{9c} represents H; —C(═O)—O—R^{16c}; an alkyl residue selected from the group consisting of methyl, ethyl, n-propyl, isopropyl, n-butyl, 2-butyl, isobutyl, tert-butyl, n-pentyl, sec-pentyl, neo-pentyl, n-hexyl, —(CH₂)—(CH₂)—NH—C(═O)—O—C(CH₃)₃, —(CH₂)—NH—C(═O)—O—C(CH₃)₃, —(CH₂)—CN, —(CH₂)—(CH₂)—CN, —(CH₂)—Cl, —(CH₂)—(CH₂)—Cl, —(CH₂)—NH₂, —(CH₂)—(CH₂)—NH₂, —(CH₂)—C(═O)—O—CH₃, —(CH₂)—(CH₂)—C(═O)—O—CH₃ and —(CH₂)—C(═O)—O—C₂H₅; for a heteroalkyl residue selected from the group consisting of —CH₂—O—CH₃, —CH₂—O—C₂H₅, —CH₂—O—CH(CH₃)₂, —CH₂—O—C(CH₃)₃, —CH₂—CH₂—O—CH₃ and —CH₂—CH₂—O—C₂H₅; for a residue selected from the group consisting of cyclopropyl, cyclobutyl, cyclopentyl, cyclohexyl, cycloheptyl, cyclopentenyl and cyclohexenyl; or a residue selected from the group consisting of pyridinyl, thiazolyl, —(CH₂)-furanyl, —(CH₂)—(CH₂)-furanyl, thienyl, phenyl, benzyl and phenethyl, which is respectively unsubstituted or is substituted with optionally 1, 2, 3, 4 or 5 substituents independently selected from the group consisting of F, Cl, Br, I, —CN, methyl, ethyl, n-propyl, isopropyl, n-butyl, 2-butyl, isobutyl, tert-butyl, —O—CH₃, —O—C₂H₅ and —O—C₃H₇;
R^{11c} represents H;
R^{12c} represents H;
R^{13c} represents H;
R^{14c} represents H;
R^{16c} represents an alkyl residue selected from the group consisting of methyl, ethyl, n-propyl, isopropyl, n-butyl, 2-butyl, isobutyl, tert-butyl, n-pentyl, sec-pentyl, neo-pentyl and n-hexyl; and
R^{21c} represents an alkyl residue selected from the group consisting of methyl, ethyl, n-propyl, isopropyl, n-butyl, 2-butyl, isobutyl, tert-butyl, n-pentyl, sec-pentyl, neo-pentyl, n-hexyl, —CF₃, —CHF₂, —CH₂F, —CF₂—CF₃, —(CH₂)—CF₃, —(CH₂)—Cl, —(CH₂)—(CH₂)—Cl, —(CH₂)—(CH₂)—(CH₂)—Cl and —(CH₂)—(CH₂)—(CH₂)—(CH₂)—Cl; or represents a residue selected from the group consisting of phenyl and benzyl, which is respectively unsubstituted or substituted with 1, 2, 3, 4 or 5 substituents independently selected from the group consisting of F, Cl, Br and —CF₃.

25. A compound as claimed in claim 1, selected from the group consisting of:
1 6-hydroxy-N-isobutyl-N-[3-(4-morpholin-4-yl-quinazolin-6-yl)-benzyl]-nicotinamide,
2 N-cyclopropyl-6-hydroxy-N-[3-(4-pyrrolidin-1-yl-quinazolin-6-yl)-benzyl]-nicotinamide,
3 N-cyclopropyl-N-[3-(4-cyclopropylamino-quinazolin-6-yl)-benzyl]-2,4-difluorobenzamide,
4 thiophene-3-carboxylic acid cyclopropyl-[3-(4-cyclopropylamino-quinazolin-6-yl)-benzyl]-amide,
5 N-isobutyl-N-[3-(4-morpholin-4-yl-quinazolin-6-yl)-benzyl]-benzamide,
6 2-bromo-N-isobutyl-N-[3-(4-morpholin-4-yl-quinazolin-6-yl)-benzyl]-benzamide,
7 N-isobutyl-3,4-dimethoxy-N-[3-(4-morpholin-4-yl-quinazolin-6-yl)-benzyl]-benzamide,
8 2,4-difluoro-N-isobutyl-N-[3-(4-morpholin-4-yl-quinazolin-6-yl)-benzyl]-benzamide,
9 isoquinoline-3-carboxylic acid isobutyl-[3-(4-morpholin-4-yl-quinazolin-6-yl)-benzyl]-amide,
10 N-isobutyl-N-[3-(4-morpholin-4-yl-quinazolin-6-yl)-benzyl]-3-phenyl-acrylamide,
11 4-chloro-N-isobutyl-N-[3-(4-morpholin-4-yl-quinazolin-6-yl)-benzyl]-benzamide,
12 4-cyano-N-isobutyl-N-[3-(4-morpholin-4-yl-quinazolin-6-yl)-benzyl]-benzamide,
13 pyrazine-2-carboxylic acid isobutyl-[3-(4-morpholin-4-yl-quinazolin-6-yl)-benzyl]-amide,
14 6-bromo-pyridine-2-carboxylic acid isobutyl-[3-(4-morpholin-4-yl-quinazolin-6-yl)-benzyl]-amide,
15 N-isobutyl-N-[3-(4-morpholin-4-yl-quinazolin-6-yl)-benzyl]-2-phenoxy-acetamide,
16 N-isobutyl-N-[3-(4-morpholin-4-yl-quinazolin-6-yl)-benzyl]-2-pyridin-2-yl-acetamide,
17 3-cyano-N-isobutyl-N-[3-(4-morpholin-4-yl-quinazolin-6-yl)-benzyl]-benzamide,
18 thiophene-3-carboxylic acid isobutyl-[3-(4-morpholin-4-yl-1 quinazolin-6-yl)-benzyl]-amide,
19 6-chloro-N-isobutyl-N-[3-(4-morpholin-4-yl-quinazolin-6-yl)-benzyl]-nicotinamide,
20 3-methyl-thiophene-2-carboxylic acid isobutyl-[3-(4-morpholin-4-yl-quinazolin-6-yl)-benzyl]-amide,
21 2-cyano-N-isobutyl-N-[3-(4-morpholin-4-yl-quinazolin-6-yl)-benzyl]-acetamide,
22 N-isobutyl-4-methyl-N-[3-(4-morpholin-4-yl-quinazolin-6-yl)-benzyl]-benzamide,
23 N-isobutyl-N-[3-(4-morpholin-4-yl-quinazolin-6-yl)-benzyl]-2-trifluoromethyl-benzamide,
24 5-bromo-thiophene-2-carboxylic acid isobutyl-[3-(4-morpholin-4-yl-quinazolin-6-yl)-benzyl]-amide,
25 N-isobutyl-N-[3-(4-morpholin-4-yl-quinazolin-6-yl)-benzyl]-isonicotinamide,
26 N-isobutyl-N-[3-(4-morpholin-4-yl-quinazolin-6-yl)-benzyl]-butyramide,
27 2-hydroxy-N-isobutyl-N-[3-(4-morpholin-4-yl-quinazolin-6-yl)-benzyl]-benzamide,
28 2-fluoro-N-isobutyl-N-[3-(4-morpholin-4-yl-quinazolin-6-yl)-benzyl]-4-trifluoromethyl-benzamide,
29 thiophene-2-carboxylic acid isobutyl-[3-(4-morpholin-4-yl-quinazolin-6-yl)-benzyl]-amide,
30 N-isobutyl-N-[3-(4-morpholin-4-yl-quinazolin-6-yl)-benzyl]-nicotinamide,
31 2-dimethylamino-N-isobutyl-N-[3-(4-morpholin-4-yl-quinazolin-6-yl)-benzyl]-acetamide,
32 N-isobutyl-4-methoxy-N-[3-(4-morpholin-4-yl-quinazolin-6-yl)-benzyl]-benzamide,
33 2-chloro-N-isobutyl-N-[3-(4-morpholin-4-yl-quinazolin-6-yl)-benzyl]-benzamide,
34 furan-2-carboxylic acid isobutyl-[3-(4-morpholin-4-yl-quinazolin-6-yl)-benzyl]-amide,
35 pyridine-2-carboxylic acid isobutyl-[3-(4-morpholin-4-yl-quinazolin-6-yl)-benzyl]-amide,
36 N-benzyl-N-[3-(4-isopropylamino-quinazolin-6-yl)-benzyl]-benzamide,
37 N-benzyl-2-bromo-N-[3-(4-isopropylamino-quinazolin-6-yl)-benzyl]-benzamide,
38 3-methyl-but-2-ene acid benzyl-[3-(4-isopropylamino-quinazolin-6-yl)-benzyl]-amide,
39 N-benzyl-N-[3-(4-isopropylamino-quinazolin-6-yl)-benzyl]-3,4-dimethoxy-benzamide,
40 N-benzyl-2,4-difluoro-N-[3-(4-isopropylamino-quinazolin-6-yl)-benzyl]-benzamide,
41 isoquinoline-3-carboxylic acid benzyl-[3-(4-isopropylamino-quinazolin-6-yl)-benzyl]-amide,
42 N-benzyl-N-[3-(4-isopropylamino-quinazolin-6-yl)-benzyl]-3-phenyl-acrylamide,
43 N-benzyl-4-chloro-N-[3-(4-isopropylamino-quinazolin-6-yl)-benzyl]-benzamide,
44 N-benzyl-4-cyano-N-[3-(4-isopropylamino-quinazolin-6-yl)-benzyl]-benzamide,
45 pyrazine-2-carboxylic acid benzyl-[3-(4-isopropylamino-quinazolin-6-yl)-benzyl]-amide,
46 6-bromo-pyridine-2-carboxylic acid benzyl-[3-(4-isopropylamino-quinazolin-6-yl)-benzyl]-amide,
47 N-benzyl-N-[3-(4-isopropylamino-quinazolin-6-yl)-benzyl]-2-phenoxy-acetamide,
48 N-benzyl-N-[3-(4-isopropylamino-quinazolin-6-yl)-benzyl]-2-pyridin-2-yl-acetamide,
49 N-benzyl-3-cyano-N-[3-(4-isopropylamino-quinazolin-6-yl)-benzyl]-benzamide,
50 thiophene-3-carboxylic acid benzyl-[3-(4-isopropylamino-quinazolin-6-yl)-benzyl]-amide,
51 N-benzyl-6-chloro-N-[3-(4-isopropylamino-quinazolin-6-yl)-benzyl]-nicotinamide,
52 3-methyl-thiophene-2-carboxylic acid benzyl-[3-(4-isopropylamino-quinazolin-6-yl)-benzyl]-amide,
53 N-benzyl-2-cyano-N-[3-(4-isopropylamino-quinazolin-6-yl)-benzyl]-acetamide,
54 N-benzyl-N-[3-(4-isopropylamino-quinazolin-6-yl)-benzyl]-4-methyl-benzamide,
55 N-benzyl-N-[3-(4-isopropylamino-quinazolin-6-yl)-benzyl]-2-trifluoromethyl-benzamide,
56 N-benzyl-2-hydroxy-N-[3-(4-isopropylamino-quinazolin-6-yl)-benzyl]-benzamide,
57 N-[3-(4-diethylamino-quinazolin-6-yl)-benzyl]-N-methyl-benzamide,
58 2-bromo-N-[3-(4-diethylamino-quinazolin-6-yl)-benzyl]-N-methyl-benzamide,
59 N-[3-(4-diethylamino-quinazolin-6-yl)-benzyl]-3,4-dimethoxy-N-methyl-benzamide,
60 N-[3-(4-diethylamino-quinazolin-6-yl)-benzyl]-2,4-difluoro-N-methyl-benzamide,
61 isoquinoline-3-carboxylic acid [3-(4-diethylamino-quinazolin-6-yl)-benzyl]-methyl-amide,
62 N-[3-(4-diethylamino-quinazolin-6-yl)-benzyl]-N-methyl-3-phenyl-acrylamide,
63 4-chloro-N-[3-(4-diethylamino-quinazolin-6-yl)-benzyl]-N-methyl-benzamide,
64 4-cyano-N-[3-(4-diethylamino-quinazolin-6-yl)-benzyl]-N-methyl-benzamide,
65 pyrazin-2-carboxylic acid [3-(4-diethylamino-quinazolin-6-yl)-benzyl]-methyl-amide,
66 6-bromo-pyridine-2-carboxylic acid [3-(4-diethylamino-quinazolin-6-yl)-benzyl]-methyl-amide,
67 N-[3-(4-diethylamino-quinazolin-6-yl)-benzyl]-N-methyl-2-phenoxy-acetamide,
68 N-[3-(4-diethylamino-quinazolin-6-yl)-benzyl]-N-methyl-2-pyridin-2-yl-acetamide,
69 3-cyano-N-[3-(4-diethylamino-quinazolin-6-yl)-benzyl]-N-methyl-benzamide,
70 thiophene-3-carboxylic acid [3-(4-diethylamino-quinazolin-6-yl)-benzyl]-methyl-amide,
71 6-chloro-N-[3-(4-diethylamino-quinazolin-6-yl)-benzyl]-N-methyl-nicotinamide,
72 3-methyl-thiophene-2-carboxylic acid [3-(4-diethylamino-quinazolin-6-yl)-benzyl]-methyl-amide,
73 N-[3-(4-diethylamino-quinazolin-6-yl)-benzyl]-6-hydroxy-N-methyl-nicotinamide,
74 2-cyano-N-[3-(4-diethylamino-quinazolin-6-yl)-benzyl]-N-methyl-acetamide,
75 N-[3-(4-diethylamino-quinazolin-6-yl)-benzyl]-4,N-dimethyl-benzamide,
76 N-[3-(4-diethylamino-quinazolin-6-yl)-benzyl]-N-methyl-2-trifluoromethyl-benzamide,
77 5-bromo-thiophene-2-carboxylic acid [3-(4-diethylamino-quinazolin-6-yl)-benzyl]-methyl-amide,
78 N-[3-(4-diethylamino-quinazolin-6-yl)-benzyl]-N-methyl-isonicotinamide,
79 N-[3-(4-diethylamino-quinazolin-6-yl)-benzyl]-N-methyl-butyramide,
80 N-[3-(4-diethylamino-quinazolin-6-yl)-benzyl]-2-hydroxy-N-methyl-benzamide,
81 N-[3-(4-diethylamino-quinazolin-6-yl)-benzyl]-2-fluoro-N-methyl-4-trifluoromethyl-benzamide,
82 thiophene-2-carboxylic acid [3-(4-diethylamino-quinazolin-6-yl)-benzyl]-methyl-amide,
83 N-[3-(4-diethylamino-quinazolin-6-yl)-benzyl]-N-methyl-nicotinamide,
84 N-[3-(4-diethylamino-quinazolin-6-yl)-benzyl]-2-dimethylamino-N-methyl-acetamide,
85 N-[3-(4-diethylamino-quinazolin-6-yl)-benzyl]-4-methoxy-N-methyl-benzamide,
86 2-chloro-N-[3-(4-diethylamino-quinazolin-6-yl)-benzyl]-N-methyl-benzamide,
87 furan-2-carboxylic acid [3-(4-diethylamino-quinazolin-6-yl)-benzyl]-methyl-amide,
88 pyridine-2-carboxylic acid [3-(4-diethylamino-quinazolin-6-yl)-benzyl]-methyl-amide,
89 N-[3-(4-phenylamino-quinazolin-6-yl)-benzyl]-N-propyl-benzamide,
90 2-phenoxy-N-[3-(4-phenylamino-quinazolin-6-yl)-benzyl]-N-propyl-acetamide,
91 4-methyl-N-[3-(4-phenylamino-quinazolin-6-yl)-benzyl]-N-propyl-benzamide,
92 N-[3-(4-phenylamino-quinazolin-6-yl)-benzyl]-N-propyl-isonicotinamide,
93 2-fluoro-N-[3-(4-phenylamino-quinazolin-6-yl)-benzyl]-N-propyl-4-trifluoromethyl-benzamide,
94 4-methoxy-N-[3-(4-phenylamino-quinazolin-6-yl)-benzyl]-N-propyl-benzamide,
95 pyridine-2-carboxylic acid [3-(4-phenylamino-quinazolin-6-yl)-benzyl]-propyl-amide,
96 3-methyl-thiophene-2-carboxylic acid cyclopropyl-[3-(4-pyrrolidin-1-yl-quinazolin-6-yl)-benzyl]-amide,
97 2-bromo-N-[3-(4-phenylamino-quinazolin-6-yl)-benzyl]-N-propyl-benzamide,
98 3,4-dimethoxy-N-[3-(4-phenylamino-quinazolin-6-yl)-benzyl]-N-propyl-benzamide,
99 2,4-difluoro-N-[3-(4-phenylamino-quinazolin-6-yl)-benzyl]-N-propyl-benzamide,
100 isoquinoline-3-carboxylic acid [3-(4-phenylamino-quinazolin-6-yl)-benzyl]-propyl-amide,
101 3-phenyl-N-[3-(4-phenylamino-quinazolin-6-yl)-benzyl]-N-propyl-acrylamide,
102 4-chloro-N-[3-(4-phenylamino-quinazolin-6-yl)-benzyl]-N-propyl-benzamide,
103 4-cyano-N-[3-(4-phenylamino-quinazolin-6-yl)-benzyl]-N-propyl-benzamide,
104 pyrazine-2-carboxylic acid [3-(4-phenylamino-quinazolin-6-yl)-benzyl]-propyl-amide,
105 6-bromo-pyridine-2-carboxylic acid [3-(4-phenylamino-quinazolin-6-yl)-benzyl]-propyl-amide,
106 N-[3-(4-phenylamino-quinazolin-6-yl)-benzyl]-N-propyl-2-pyridin-2-yl-acetamide,
107 3-cyano-N-[3-(4-phenylamino-quinazolin-6-yl)-benzyl]-N-propyl-benzamide,
108 thiophene-3-carboxylic acid [3-(4-phenylamino-quinazolin-6-yl)-benzyl]-propyl-amide,
109 6-chloro-N-[3-(4-phenylamino-quinazolin-6-yl)-benzyl]-N-propyl-nicotinamide,
110 3-methyl-thiophene-2-carboxylic acid [3-(4-phenylamino-quinazolin-6-yl)-benzyl]-propyl-amide,
111 6-hydroxy-N-[3-(4-phenylamino-quinazolin-6-yl)-benzyl]-N-propyl-nicotinamide,
112 2-cyano-N-[3-(4-phenylamino-quinazolin-6-yl)-benzyl]-N-propyl-acetamide,
113 N-[3-(4-phenylamino-quinazolin-6-yl)-benzyl]-N-propyl-2-trifluoromethyl-benzamide,
114 5-bromo-thiophene-2-carboxylic acid [3-(4-phenylamino-quinazolin-6-yl)-benzyl]-propyl-amide,
115 N-[3-(4-phenylamino-quinazolin-6-yl)-benzyl]-N-propyl-butyramide,
116 thiophene-2-carboxylic acid [3-(4-phenylamino-quinazolin-6-yl)-benzyl]-propyl-amide,
117 N-[3-(4-phenylamino-quinazolin-6-yl)-benzyl]-N-propyl-nicotinamide,
118 2-dimethylamino-N-[3-(4-phenylamino-quinazolin-6-yl)-benzyl]-N-propyl-acetamide,
119 2-chloro-N-[3-(4-phenylamino-quinazolin-6-yl)-benzyl]-N-propyl-benzamide,
120 furan-2-carboxylic acid [3-(4-phenylamino-quinazolin-6-yl)-benzyl]-propyl-amide,
121 N-cyclopropyl-N-[3-(4-pyrrolidin-1-yl-quinazolin-6-yl)-benzyl]-benzamide,
122 2-bromo-N-cyclopropyl-N-[3-(4-pyrrolidin-1-yl-quinazolin-6-yl)-benzyl]-benzamide,
123 N-cyclopropyl-3,4-dimethoxy-N-[3-(4-pyrrolidin-1-yl-quinazolin-6-yl)-benzyl]-benzamide,
124 N-cyclopropyl-2,4-difluoro-N-[3-(4-pyrrolidin-1-yl-quinazolin-6-yl)-benzyl]-benzamide,
125 isoquinoline-3-carboxylic acid cyclopropyl-[3-(4-pyrrolidin-1-yl-quinazolin-6-yl)-benzyl]-amide,
126 N-cyclopropyl-3-phenyl-N-[3-(4-pyrrolidin-1-yl-quinazolin-6-yl)-benzyl]-acrylamide,
127 4-chloro-N-cyclopropyl-N-[3-(4-pyrrolidin-1-yl-quinazolin-6-yl)-benzyl]-benzamide,
128 4-cyano-N-cyclopropyl-N-[3-(4-pyrrolidin-1-yl-quinazolin-6-yl)-benzyl]-benzamide,
129 pyrazine-2-carboxylic acid cyclopropyl-[3-(4-pyrrolidin-1-yl-quinazolin-6-yl)-benzyl]-amide,
130 6-bromo-pyridine-2-carboxylic acid cyclopropyl-[3-(4-pyrrolidin-1-yl-quinazolin-6-yl)-benzyl]-amide,
131 thiophene-3-carboxylic acid cyclopropyl-[3-(4-pyrrolidin-1-yl-quinazolin-6-yl)-benzyl]-amide,
132 6-chloro-N-cyclopropyl-N-[3-(4-pyrrolidin-1-yl-quinazolin-6-yl)-benzyl]-nicotinamide,
133 2-cyano-N-cyclopropyl-N-[3-(4-pyrrolidin-1-yl-quinazolin-6-yl)-benzyl]-acetamide,
134 N-cyclopropyl-4-methyl-N-[3-(4-pyrrolidin-1-yl-quinazolin-6-yl)-benzyl]-benzamide,
135 N-cyclopropyl-N-[3-(4-pyrrolidin-1-yl-quinazolin-6-yl)-benzyl]-2-trifluoromethyl-benzamide,
136 5-bromo-thiophene-2-carboxylic acid cyclopropyl-[3-(4-pyrrolidin-1-yl-quinazolin-6-yl)-benzyl]-amide,
137 N-cyclopropyl-N-[3-(4-pyrrolidin-1-yl-quinazolin-6-yl)-benzyl]-isonicotinamide,
138 N-cyclopropyl-N-[3-(4-pyrrolidin-1-yl-quinazolin-6-yl)-benzyl]-butyramide,
139 N-cyclopropyl-2-fluoro-N-[3-(4-pyrrolidin-1-yl-quinazolin-6-yl)-benzyl]-4-trifluoromethyl-benzamide,
140 thiophene-2-carboxylic acid cyclopropyl-[3-(4-pyrrolidin-1-yl-quinazolin-6-yl)-benzyl]-amide,
141 N-cyclopropyl-N-[3-(4-pyrrolidin-1-yl-quinazolin-6-yl)-benzyl]-nicotinamide,
142 N-cyclopropyl-4-methoxy-N-[3-(4-pyrrolidin-1-yl-quinazolin-6-yl)-benzyl]-benzamide,
143 2-chloro-N-cyclopropyl-N-[3-(4-pyrrolidin-1-yl-quinazolin-6-yl)-benzyl]-benzamide,
144 furan-2-carboxylic acid cyclopropyl-[3-(4-pyrrolidin-1-yl-quinazolin-6-yl)-benzyl]-amide,
145 N-cyclopropyl-2-phenoxy-N-[3-(4-pyrrolidin-1-yl-quinazolin-6-yl)-benzyl]-acetamide,
146 3-cyano-N-cyclopropyl-N-[3-(4-pyrrolidin-1-yl-quinazolin-6-yl)-benzyl]-benzamide,
147 pyridine-2-carboxylic acid cyclopropyl-[3-(4-pyrrolidin-1-yl-quinazolin-6-yl)-benzyl]-amide,
148 N-benzyl-N-[3-(4-isopropylamino-quinazolin-6-yl)-benzyl]-butyramide,
149 furan-2-carboxylic acid benzyl-[3-(4-isopropylamino-quinazolin-6-yl)-benzyl]-amide,
150 N-benzyl-2-chloro-N-[3-(4-isopropylamino-quinazolin-6-yl)-benzyl]-benzamide,
151 N-benzyl-N-[3-(4-isopropylamino-quinazolin-6-yl)-benzyl]-isonicotinamide,
152 N-benzyl-N-[3-(4-cyclopropylamino-quinazolin-6-yl)-benzyl]-2-fluoro-4-trifluoromethyl-benzamide,
153 N-benzyl-N-[3-(4-isopropylamino-quinazolin-6-yl)-benzyl]-nicotinamide,
154 thiophene-2-carboxylic acid benzyl-[3-(4-isopropylamino-quinazolin-6-yl)-benzyl]-amide,
155 N-benzyl-N-[3-(4-isopropylamino-quinazolin-6-yl)-benzyl]-4-methoxy-benzamide,
156 N-cyclopropyl-N-[3-(4-cyclopropylamino-quinazolin-6-yl)-benzyl]-2-hydroxy-benzamide,
157 2-bromo-N-cyclopropyl-N-[3-(4-cyclopropylamino-quinazolin-6-yl)-benzyl]-benzamide,
158 N-cyclopropyl-N-[3-(4-cyclopropylamino-quinazolin-6-yl)-benzyl]-3,4-dimethoxy-benzamide,
159 isoquinoline-3-carboxylic acid cyclopropyl-[3-(4-cyclopropylamino-quinazolin-6-yl)-benzyl]-amide,
160 4-chloro-N-cyclopropyl-N-[3-(4-cyclopropylamino-quinazolin-6-yl)-benzyl]-benzamide,
161 4-cyano-N-cyclopropyl-N-[3-(4-cyclopropylamino-quinazolin-6-yl)-benzyl]-benzamide,
162 pyrazine-2-carboxylic acid cyclopropyl-[3-(4-cyclopropylamino-quinazolin-6-yl)-benzyl]-amide,
163 6-bromo-pyridine-2-carboxylic acid cyclopropyl-[3-(4-cyclopropylamino-quinazolin-6-yl)-benzyl]-amide,
164 N-cyclopropyl-N-[3-(4-cyclopropylamino-quinazolin-6-yl)-benzyl]-2-phenoxy-acetamide,
165 N-cyclopropyl-N-[3-(4-cyclopropylamino-quinazolin-6-yl)-benzyl]-isonicotinamide,
166 5-bromo-thiophene-2-carboxylic acid cyclopropyl-[3-(4-cyclopropylamino-quinazolin-6-yl)-benzyl]-amide,
167 N-cyclopropyl-N-[3-(4-cyclopropylamino-quinazolin-6-yl)-benzyl]-2-trifluoromethyl-benzamide,
168 N-cyclopropyl-N-[3-(4-cyclopropylamino-quinazolin-6-yl)-benzyl]-butyramide,
169 furan-2-carboxylic acid cyclopropyl-[3-(4-cyclopropylamino-quinazolin-6-yl)-benzyl]-amide,
170 pyridine-2-carboxylic acid cyclopropyl-[3-(4-cyclopropylamino-quinazolin-6-yl)-benzyl]-amide,
171 N-cyclopropyl-N-[3-(4-cyclopropylamino-quinazolin-6-yl)-benzyl]-4-methoxy-benzamide,
172 2-chloro-N-cyclopropyl-N-[3-(4-cyclopropylamino-quinazolin-6-yl)-benzyl]-benzamide,
173 N-cyclopropyl-N-[3-(4-cyclopropylamino-quinazolin-6-yl)-benzyl]-2-dimethylamino-acetamide,
174 N-cyclopropyl-N-[3-(4-cyclopropylamino-quinazolin-6-yl)-benzyl]-2-pyridin-2-yl-acetamide,
175 N-[3-(4-cyclopropylamino-quinazolin-6-yl)-benzyl]-4,N-dimethyl-benzamide,
176 3-cyano-N-cyclopropyl-N-[3-(4-cyclopropylamino-quinazolin-6-yl)-benzyl]-benzamide,
177 N-cyclopropyl-N-[3-(4-cyclopropylamino-quinazolin-6-yl)-benzyl]-2-fluoro-4-trifluoromethyl-benzamide,
178 thiophene-2-carboxylic acid cyclopropyl-[3-(4-cyclopropylamino-quinazolin-6-yl)-benzyl]-amide,
179 6-chloro-N-cyclopropyl-N-[3-(4-cyclopropylamino-quinazolin-6-yl)-benzyl]-nicotinamide,
180 1-[3-(4-cyclopropylamino-2-methyl-quinazolin-6-yl)-benzyl]-pyrrolidine-2,5-dione,
181 1-[3-(4-cyclopropylamino-2-trifluoromethyl-quinazolin-6-yl)-benzyl]-pyrrolidine-2,5-dione,
182 1-[3-(2,4-bis-cyclopropylamino-quinazolin-6-yl)-benzyl]-pyrrolidine-2,5-dione,
183 1-[3-(2-amino-4-cyclopropylamino-quinazolin-6-yl)-benzyl]-pyrrolidine-2,5-dione,
184 1-[3-(4-cyclopropylamino-2-methylamino-quinazolin-6-yl)-benzyl]-pyrrolidine-2,5-dione,
185 1-[3-(4-cyclopropylamino-2-phenylamino-quinazolin-6-yl)-benzyl]-pyrrolidine-2,5-dione,
186 1-[3-(4-cyclopropylamino-2-dimethylamino-quinazolin-6-yl)-benzyl]-pyrrolidine-2,5-dione,
187 1-[3-(2-tert-butyl-4-cyclopropylamino-quinazolin-6-yl)-benzyl]-pyrrolidine-2,5-dione,
188 N-tert-butyl-2-cyano-N-(5-(4-(cyclopropylamino)quinazolin-6-yl)-2-fluorobenzyl)acetamide,
189 4-(5-(4-(cyclopropylamino)quinazolin-6-yl)-2-fluorobenzyl)morpholine-3,5-dione,
190 methyl 2-(2-cyano-N-(3-(4-(cyclopropylamino)quinazolin-6-yl)benzyl)acetamido)acetate,
191 N-cyclopropyl-N-(3-(4-(cyclopropylamino)quinazolin-6-yl)-2-fluorobenzyl)acetamide,
192 1-(5-(4-(cyclopropylamino)quinazolin-6-yl)-2-fluorobenzyl)-1H-pyrrole-2,5-dione,
193 1-(5-(4-(cyclopropylamino)quinazolin-6-yl)-2-fluorobenzyl)pyrrolidine-2,5-dione,
194 1-[3-(4-cyclopropylamino-quinazolin-6-yl)-benzyl]-piperidine-2,6-dione,
195 2-cyano-N-[3-(4-cyclopropylamino-quinazolin-6-yl)-benzyl]-N-pyridin-3-yl-acetamide,
196 1-[3-(4-tert-butylamino-quinazolin-6-yl)-benzyl]-pyrrolidine-2,5-dione hydrochloride,
197 2-cyano-N-cyclopropyl-N-[3-(4-cyclopropylamino-quinazolin-6-yl)-2-fluoro-benzyl]-acetamide,
198 1-[3-(4-methylamino-quinazolin-6-yl)-benzyl]-pyrrolidine-2,5-dione,
199 1-(3-(4-(benzylamino)quinazolin-6-yl)benzyl)pyrrolidine-2,5-dione,
200 N-cyanomethyl-N-[3-(4-cyclopropylamino-quinazolin-6-yl)-benzyl]-acetamide,
201 N-[5-(4-tert-butylamino-quinazolin-6-yl)-2-fluoro-benzyl]-2-cyano-N-cyclopropyl-acetamide,
202 N-tert-butyl-N-[3-(4-cyclopropylamino-quinazolin-6-yl)-benzyl]-acetamide,
203 {acetyl-[3-(4-cyclopropylamino-quinazolin-6-yl)-benzyl]-amino}-methyl acetate,
204 tert-butyl 2-(N-(3-(4-(cyclopropylamino)quinazolin-6-yl)benzyl)acetamido) ethylcarbamate,
205 N-cyclopropyl-N-[3-(4-cyclopropylamino-quinazolin-6-yl)-benzyl]-3,3,3-trifluoro-propionamide hydrochloride,
206 cyclopropyl-{6-[3-(1,1-dioxo-1λ⁶-isothiazolidin-2-yl methyl)-phenyl]-quinazoline-4-yl}-amine hydrochloride,
207 N-(3-(4-(cyclopropylamino)quinazolin-6-yl)benzyl)-N-phenylacetamide hydrochloride,
208 N-cyclopropyl-N-[3-(4-cyclopropylamino-quinazolin-6-yl)-benzyl]-2-fluoro-acetamide,
209 N-cyclopentyl-N-(3-(4-(cyclopropylamino)quinazolin-6-yl)benzyl)acetamide,
210 N-cyclopentyl-N-(3-(4-(cyclopropylamino)quinazolin-6-yl)benzyl)methane sulfonamide,
211 2-cyano-N-cyclopropyl-N-[3-(4-cyclopropylamino-quinazolin-6-yl)-benzyl]-acetamide,
212 4-(3-(4-(cyclopropylamino)quinazolin-6-yl)benzyl)morpholine-3,5-dione,
213 N-(3-(4-(cyclopropylamino)quinazolin-6-yl)benzyl)-N-(2-methoxyethyl)acetamide,
214 N-[3-(4-cyclopropylamino-quinazolin-6-yl)-benzyl]-N-phenethyl-acetamide,
215 N-(3-(4-(cyclopropylamino)quinazolin-6-yl)benzyl)-N-(furan-2-yl-methyl)methane sulfonamide,
216 N-[3-(4-cyclopropylamino-quinazolin-6-yl)-benzyl]-N-furan-2-yl-methyl-acetamide,
217 1-[3-(4-cyclopropylamino-quinazolin-6-yl)-benzyl]-pyrrolidine-2-one hydrochloride,
218 N-butyl-N-[3-(4-cyclopropylamino-quinazolin-6-yl)-benzyl]-acetamide hydrochloride,
219 N-benzyl-N-[3-(4-cyclopropylamino-quinazolin-6-yl)-benzyl]-acetamide hydrochloride,
220 1-[3-(4-cyclopropylamino-quinazolin-6-yl)-benzyl]-3-phenyl-piperidine-2,6-dione hydrochloride,
221 N-[3-(4-cyclopropylamino-quinazolin-6-yl)-benzyl]-N-methyl-acetamide,
222 N-benzyl-N-(3-(4-(cyclopropylamino)quinazolin-6-yl)benzyl)methane sulfonamide,
223 N-(3-(4-(cyclopropylamino)quinazolin-6-yl)benzyl)-N-methyl methane sulfonamide,
224 1-[3-(4-cyclopropylamino-quinazolin-6-yl)-benzyl]-4-methyl-piperidine-2,6-dione hydrochloride,
225 N-cyclopropyl-N-(3-(4-(cyclopropylamino)quinazolin-6-yl)benzyl)-2,2,2-trifluoroethane sulfonamide,
226 N-cyclopropyl-N-(3-(4-(cyclopropylamino)quinazolin-6-yl)benzyl)-4-(trifluoromethyl)benzole sulfonamide,
227 N-cyclopropyl-N-(3-(4-(cyclopropylamino)quinazolin-6-yl)benzyl)benzole sulfonamide,
228 2-[3-(4-cyclopropylamino-quinazolin-6-yl)-benzyl]-hexahydro-isoindole-1,3-dione hydrochloride,
229 1-[3-(4-cyclopropylamino-quinazolin-6-yl)-benzyl]-3-phenyl-pyrrolidine-2,5-dione hydrochloride,
230 N-cyclopropyl-N-[5-(4-cyclopropylamino-quinazolin-6-yl)-pyridin-3-yl-methyl]-benzamide hydrochloride,
231 N-cyclopropyl-N-(1-(3-(4-(cyclopropylamino)quinazolin-6-yl)phenyl)ethyl)methane sulfonamide,
233 N-cyclopropyl-N-{1-[3-(4-cyclopropylamino-quinazolin-6-yl)-phenyl]-ethyl}-benzamide,
234 N-cyclopropyl-N-[5-(4-cyclopropylamino-quinazolin-6-yl)-pyridin-3-yl-methyl]-methane sulfonamide,
235 N-cyclopropyl-N-{1-[3-(4-cyclopropylamino-quinazolin-6-yl)-phenyl]-ethyl}-acetamide,
236 N-{1-[3-(4-cyclopropylamino-quinazolin-6-yl)-benzyl]-2,5-dioxo-pyrrolidine-3-yl}-2,2,2-trifluoro-acetamide hydrochloride,
237 N-cyclopropyl-N-[3-(4-cyclopropylamino-quinazolin-6-yl)-benzyl]-methyl malonate,
238 N-cyclopropyl-N-[3-(4-cyclopropylamino-quinazolin-6-yl)-benzyl]-methyl oxalate,
239 N-cyclopropyl-N-[3-(4-cyclopropylamino-quinazolin-6-yl)-5-methyl-benzyl]-acetamide,
240 N-cyclopropyl-N-[5-(4-cyclopropylamino-quinazolin-6-yl)-pyridin-3-yl-methyl]-acetamide,
241 2-[3-(4-cyclopropylamino-quinazolin-6-yl)-benzyl]-isoindole-1,3-dione hydrochloride,
242 2-[3-(4-cyclopropylamino-quinazolin-6-yl)-benzyl]-hexahydro-isoindole-1,3-dione hydrochloride,
243 8-[3-(4-cyclopropylamino-quinazolin-6-yl)-benzyl]-8-aza-spiro[4.5]decane-7,9-dione hydrochloride,
244 N-cyclopropyl-N-[3-(4-cyclopropylamino-quinazolin-6-yl)-2,6-difluoro-benzyl]-benzamide,
245 1-[3-(4-cyclopropylamino-quinazolin-6-yl)-benzyl]-3-methyl-pyrrolidine-2,5-dione,
246 N-cyclopropyl-N-(3-(4-(cyclopropylamino)quinazolin-6-yl)-2,6-difluorobenzyl)methane sulfonamide,
247 1-cyclopropyl-1-[3-(4-cyclopropylamino-quinazolin-6-yl)-benzyl]-3-isopropyl-urea,
248 N-cyclopropyl-N-[3-(4-cyclopropylamino-quinazolin-6-yl)-benzyl]-nicotinamide,
249 N-cyclopropyl-N-[3-(4-cyclopropylamino-quinazolin-6-yl)-2,6-difluoro-benzyl]-acetamide,
250 N-cyclopropyl-N-(3-(4-(cyclopropylamino)quinazolin-6-yl)-4-methoxybenzyl)methane sulfonamide,
251 N-cyclopropyl-N-(5-(4-(cyclopropylamino)quinazolin-6-yl)-2-fluorobenzyl)methane sulfonamide,
252 N-cyclopropyl-N-[3-(4-cyclopropylamino-quinazolin-6-yl)-benzyl]-2-methoxy-acetamide,
253 isoxazole-5-carboxylic acid cyclopropyl-[3-(4-cyclopropylamino-quinazolin-6-yl)-benzyl]-amide,
254 N-cyclopropyl-N-[3-(4-cyclopropylamino-quinazolin-6-yl)-benzyl]-2,3,4,5,6-pentafluoro-benzamide,
255 N-cyclopropyl-N-[3-(4-cyclopropylamino-quinazolin-6-yl)-benzyl]-3-phenyl-acrylamide,
256 pentanoic acid cyclopropyl-[3-(4-cyclopropylamino-quinazolin-6-yl)-benzyl]-amide,
257 N-cyclopropyl-N-[3-(4-cyclopropylamino-quinazolin-6-yl)-benzyl]-isonicotinamide,
258 1-[3-(4-cyclopropylamino-quinazolin-6-yl)-benzyl]-pyrrolidine-2,5-dione,
259 N-cyclopropyl-N-[5-(4-cyclopropylamino-quinazolin-6-yl)-2-methoxy-benzyl]-acetamide,
260 N-cyclopropyl-N-[5-(4-cyclopropylamino-quinazolin-6-yl)-2-fluoro-benzyl]-acetamide,
261 N-cyclopropyl-N-[3-(4-cyclopropylamino-quinazolin-6-yl)-4-methoxy-benzyl]-acetamide,
262 N-cyclopropyl-N-[4-(4-cyclopropylamino-quinazolin-6-yl)-thiophen-2-ylmethyl]-acetamide,
263 N-cyclopropyl-N-[3-(4-cyclopropylamino-quinazolin-6-yl)-benzyl]-3-phenyl-propionamide,
264 N-cyclopropyl-N-[3-(4-cyclopropylamino-quinazolin-6-yl)-benzyl]-2-phenyl-acetamide,
265 N-cyclopropyl-N-(3-(4-(cyclopropylamino)quinazolin-6-yl)benzyl)cyclohexane carboxamide,
266 N-cyclopropyl-N-(3-(4-(cyclopropylamino)quinazolin-6-yl)benzyl)pivalamide,
267 N-cyclopropyl-N-(3-(4-(cyclopropylamino)quinazolin-6-yl)-4-methoxybenzyl)benzamide hydrochloride,
268 N-cyclopropyl-N-(3-(4-(cyclopropylamino)quinazolin-6-yl)-4-methoxybenzyl)benzamide,
269 N-cyclopropyl-N-(5-(4-(cyclopropylamino)quinazolin-6-yl)-2-fluorobenzyl)benzamide,
270 N-cyclopropyl-N-(3-(4-(cyclopropylamino)quinazolin-6-yl)benzyl)methane sulfonamide,
271 N-cyclopropyl-N-(3-(4-(cyclopropylamino)quinazolin-6-yl)benzyl)acetamide,
272 N-cyclopropyl-N-(3-(4-(cyclopropyl(methyl)amino)quinazolin-6-yl)benzyl)picolinamide,
273 N-cyclopropyl-N-(3-(4-(cyclopropylamino)quinazolin-6-yl)benzyl)-2,2,2-trifluoroacetamide,
274 N-cyclopropyl-N-(3-(4-(cyclopropylamino)quinazolin-6-yl)benzyl)benzamide,
275 N-cyclopropyl-N-(3-(4-(cyclopropylamino)quinazolin-6-yl)benzyl)picolinamide,
276 1-(3-(4-(benzylamino)quinazolin-6-yl)benzyl)pyrrolidine-2,5-dione,
277 2-cyano-N-cyclopropyl-N-(5-(4-(cyclopropylamino)quinazolin-6-yl)-2-fluorobenzyl)acetamide,
278 1-(5-(4-(cyclopropylamino)quinazolin-6-yl)-2-fluorobenzyl)piperidine-2,6-dione,
279 N-tert-butyl-N-(5-(4-(cyclopropylamino)quinazolin-6-yl)-2-fluorobenzyl)acetamide,
280 tert-butyl 2-(2-cyano-N-(3-(4-(cyclopropylamino)quinazolin-6-yl)benzyl)acetamido)ethylcarbamate,
281 2-cyano-N-cyclopropyl-N-(5-(4-(cyclopropylamino)quinazolin-6-yl)-2-fluorobenzyl)acetamide hydrochloride,
282 N-cyclopropyl-N-(5-(4-(cyclopropylamino)quinazolin-6-yl)-2-fluorobenzyl)-3,3,3-trifluoropropanamide,
283 2-chloro-N-cyclopropyl-N-(5-(4-(cyclopropylamino)quinazolin-6-yl)-2-fluorobenzyl)acetamide,
284 N-(2-aminoethyl)-2-cyano-N-(3-(4-(cyclopropylamino)quinazolin-6-yl)benzyl)acetamide hydrochloride,
285 1-(3-(4-(dimethylamino)quinazolin-6-yl)benzyl)pyrrolidine-2,5-dione hydrochloride,
286 1-(3-(4-(butylamino)quinazolin-6-yl)benzyl)pyrrolidine-2,5-dione hydrochloride,
287 1-(3-(4-(cyclopentylamino)quinazolin-6-yl)benzyl)pyrrolidine-2,5-dione hydrochloride,
288 1-(3-(2-benzyl-4-(cyclopropylamino)quinazolin-6-yl)benzyl)pyrrolidine-2,5-dione,
289 1-(3-(4-(cyclopropylamino)-2-(pyridin-2-yl)quinazolin-6-yl)benzyl)pyrrolidine-2,5-dione,
290 1-(3-(4-(cyclopropylamino)-2-morpholinoquinazolin-6-yl)benzyl)pyrrolidine-2,5-dione,
291 1-(3-(2-(benzylamino)-4-(cyclopropylamino)quinazolin-6-yl)benzyl)pyrrolidine-2,5-dione,
292 N-cyclopropyl-6-(3-(indolin-1-ylmethyl)phenyl)quinazoline-4-amine,
293 1-(3-(4-(cyclopropylamino)-2-phenylquinazolin-6-yl)benzyl)pyrrolidine-2,5-dione,
294 N-cyclopropyl-6-(3-((cyclopropylamino)methyl)-phenyl)quinazoline-4-amine,
295 tert-butyl 3-(4-(cyclopropylamino)quinazolin-6-yl)benzylcarbamate,
296 N-cyclopropyl-6-(3-((dimethylamino)methyl)phenyl)quinazoline-4-amine,
297 6-(3-(aminomethyl)phenyl)-N-cyclopropylquinazoline-4-amine dihydrochloride,
298 N-cyclopropyl-6-(3-((cyclopropylamino)methyl)-4-methoxyphenyl)quinazoline-4-amine,
299 N-cyclopropyl-6-(3-((cyclopropylamino)methyl)-4-fluorophenyl)quinazoline-4-amine,
300 N-cyclopropyl-6-(5-((cyclopropylamino)methyl)thiophen-3-yl)quinazoline-4-amine,
301 N-cyclopropyl-6-(5-((cyclopropylamino)methyl)-2-methoxyphenyl)quinazoline-4-amine,
302 N-(3-(4-(cyclopropylamino)quinazolin-6-yl)benzyl)acetamide,
303 N-(3-(4-(cyclopropylamino)quinazolin-6-yl)benzyl)benzamide,
304 N-cyclopropyl-6-(3-((cyclopropylamino)methyl)-2,4-difluorophenyl)quinazoline-4-amine,
305 N-cyclopropyl-6-(5-((cyclopropylamino)methyl)pyridin-3-yl)quinazoline-4-amine,
306 N-cyclopropyl-6-(3-((cyclopropylamino)methyl)-5-methylphenyl)quinazoline-4-amine,
307 N-cyclopropyl-6-(3-(1-(cyclopropylamino)ethyl)phenyl)quinazoline-4-amine,
308 6-(3-((benzylamino)methyl)phenyl)-N-cyclopropylquinazoline-4-amine,
309 N-cyclopropyl-6-(3-((methylamino)methyl)phenyl)quinazoline-4-amine,
310 6-(3-((butylamino)methyl)phenyl)-N-cyclopropylquinazoline-4-amine,
311 N-(6-(3-(acetamidomethyl)phenyl)quinazoline-4-yl)-N-cyclopropylacetamide hydrochloride,
312 N-cyclopropyl-6-(3-((furan-2-ylmethylamino)methyl)phenyl)quinazoline-4-amine dihydrochloride,
313 N-cyclopropyl-6-(3-((phenethylamino)methyl)phenyl)quinazoline-4-amine,
314 N-cyclopropyl-6-(3-((2-methoxyethylamino)methyl)phenyl)quinazoline-4-amine dihydrochloride,
315 6-(3-((2-chloroethylamino)methyl)phenyl)-N-cyclopropylquinazoline-4-amine,
316 3-chloro-N-(3-(4-(cyclopropylamino)quinazolin-6-yl)benzyl)propane-1-sulfonamide hydrochloride,
317 5-chloro-N-(3-(4-(cyclopropylamino)quinazolin-6-yl)benzyl)pentanamide hydrochloride,
318 6-(3-((cyclopentylamino)methyl)phenyl)-N-cyclopropylquinazoline-4-amine,
319 N-cyclopropyl-6-(3-((phenylamino)methyl)phenyl)quinazoline-4-amine,
320 2-(3-(4-(cyclopropylamino)quinazolin-6-yl)benzylamino)acetonitrile,
321 methyl 2-(3-(4-(cyclopropylamino)quinazolin-6-yl)benzylamino)acetate,
322 N-tert-butyl-6-(3-((cyclopropylamino)methyl)-4-fluorophenyl)quinazoline-4-amine,
323 6-(3-((tert-butylamino)methyl)phenyl)-N-cyclopropylquinazoline-4-amine,
324 N-cyclopropyl-6-(3-((cyclopropylamino)methyl)-2-fluorophenyl)quinazoline-4-amine,
325 N-cyclopropyl-6-(3-((pyridin-3-ylamino)methyl)phenyl)quinazoline-4-amine,
326 tert-butyl 2-(3-(4-(cyclopropylamino)quinazolin-6-yl)benzylamino)ethylcarbamate,
327 6-(3-((tert-butylamino)methyl)-4-fluorophenyl)-N-cyclopropylquinazoline-4-amine
328 2-(2-(3-(4-(cyclopropylamino)quinazolin-6-yl)benzylamino)-2-oxoethyl)benzoic acid,
329 4-oxo-4-(3-(4-(phenylamino)quinazolin-6-yl)benzylamino)butyric acid, and
330 4-(3-(4-(carboxymethylamino)quinazolin-6-yl)benzylamino)-4-oxobutyric acid;
in each case optionally in the form of one of their pure stereoisomers, in particular enantiomers or diastereomers, their racemates, or in the form of a mixture of stereoisomers, in particular the enantiomers and/or diastereomers, in any mixture ratio, or respectively in the form of corresponding salts or respectively in the form of corresponding solvates.

23. Compounds as claimed in claim 1-21 selected from the group consisting of
331 N-benzyl-5-bromo-N-(3-(4-(isopropylamino)quinazolin-6-yl)benzyl)thiophene-2-carboxamide,
332 N-benzyl-N-(3-(4-(isopropylamino)quinazolin-6-yl)benzyl)picolinamide,
333 N-(3-(4-(cyclopropylamino)quinazolin-6-yl)benzyl)picolinamide,
334 2-chloro-N-(3-(4-(cyclopropylamino)quinazolin-6-yl)benzyl)benzamide,
335 N-(3-(4-(cyclopropylamino)quinazolin-6-yl)benzyl)-4-methoxybenzamide,
336 N-(3-(4-(cyclopropylamino)quinazolin-6-yl)benzyl)thiophene-2-carboxamide,
337 N-(3-(4-(cyclopropylamino)quinazolin-6-yl)benzyl)-2-fluoro-4-(trifluoromethyl)benzamide,
338 N-(3-(4-(cyclopropylamino)quinazolin-6-yl)benzyl)butyramide,
339 5-bromo-N-(3-(4-(cyclopropylamino)quinazolin-6-yl)benzyl)thiophene-2-carboxamide,
340 N-(3-(4-(cyclopropylamino)quinazolin-6-yl)benzyl)-2-(trifluoromethyl)benzamide,
341 N-(3-(4-(cyclopropylamino)quinazolin-6-yl)benzyl)-4-methylbenzamide,
342 6-chloro-N-(3-(4-(cyclopropylamino)quinazolin-6-yl)benzyl)nicotinamide,
343 N-(3-(4-(cyclopropylamino)quinazolin-6-yl)benzyl)thiophene-3-carboxamide,
344 N-(3-(4-(cyclopropylamino)quinazolin-6-yl)benzyl)-2-phenoxyacetamide,
345 6-bromo-N-(3-(4-(cyclopropylamino)quinazolin-6-yl)benzyl)picolinamide,
346 4-cyano-N-(3-(4-(cyclopropylamino)quinazolin-6-yl)benzyl)benzamide,
347 N-(3-(4-(cyclopropylamino)quinazolin-6-yl)benzyl)cinnamamide,
348 N-(3-(4-(cyclopropylamino)quinazolin-6-yl)benzyl)isoquinoline-3-carboxamide,
349 N-(3-(4-(cyclopropylamino)quinazolin-6-yl)benzyl)-2,4-difluorobenzamide,
350 2-bromo-N-(3-(4-(cyclopropylamino)quinazolin-6-yl)benzyl)benzamide,
351 N-(3-(4-(cyclopropylamino)quinazolin-6-yl)benzyl)furan-2-carboxamide,
352 N-(3-(4-(cyclopropylamino)quinazolin-6-yl)benzyl)-2-(dimethylamino)acetamide,
353 N-(3-(4-(cyclopropylamino)quinazolin-6-yl)benzyl)nicotinamide,
354 N-(3-(4-(cyclopropylamino)quinazolin-6-yl)benzyl)-2-hydroxybenzamide,
355 N-(3-(4-(cyclopropylamino)quinazolin-6-yl)benzyl)isonicotinamide,
356 2-cyano-N-(3-(4-(cyclopropylamino)quinazolin-6-yl)benzyl)acetamide,
357 N-(3-(4-(cyclopropylamino)quinazolin-6-yl)benzyl)-3-methylthiophene-2-carboxamide,
358 3-cyano-N-(3-(4-(cyclopropylamino)quinazolin-6-yl)benzyl)benzamide,
359 N-(3-(4-(cyclopropylamino)quinazolin-6-yl)benzyl)-2-(pyridin-2-yl)acetamide,
360 N-(3-(4-(cyclopropylamino)quinazolin-6-yl)benzyl)pyrazine-2-carboxamide,
361 4-chloro-N-(3-(4-(cyclopropylamino)quinazolin-6-yl)benzyl)benzamide,
362 N-(3-(4-(cyclopropylamino)quinazolin-6-yl)benzyl)-3,4-dimethoxybenzamide,
363 N-cyclopropyl-N-(3-(4-(3-methylbenzylamino)quinazolin-6-yl)benzyl)benzole sulfonamide,
364 N-cyclopropyl-2-fluoro-N-(3-(4-(3-methylbenzylamino)quinazolin-6-yl)benzyl)-4-(trifluoromethyl)benzamide,
365 N-cyclopropyl-N-(3-(4-(3-methylbenzylamino)quinazolin-6-yl)benzyl)-2-phenylacetamide,
366 N-cyclopropyl-N-(5-(4-(cyclopropylamino)quinazolin-6-yl)-2-fluorobenzyl)-3,4-dimethoxybenzamide,
367 N-cyclopropyl-N-(5-(4-(cyclopropylamino)quinazolin-6-yl)-2-fluorobenzyl)nicotinamide,
368 N-cyclopropyl-N-(5-(4-(cyclopropylamino)quinazolin-6-yl)-2-fluorobenzyl)benzole sulfonamide,
369 N-cyclopropyl-1,3-dimethyl-N-(3-(4-(phenethylamino)quinazolin-6-yl)benzyl)-1H-pyrazole-5-carboxamide,
370 N-cyclopropyl-N-(3-(4-(phenethylamino)quinazolin-6-yl)benzyl)picolinamide,
371 N-cyclopropyl-4-methoxy-N-(3-(4-(3-methylbenzylamino)quinazolin-6-yl)benzyl)benzole sulfonamide,
372 N-cyclopropyl-N-(3-(4-(3-methylbenzylamino)quinazolin-6-yl)benzyl)-3-(trifluoromethyl)benzamide,
373 N-cyclopropyl-N-(3-(4-(3-methylbenzylamino)quinazolin-6-yl)benzyl)-3-phenylpropanamide,
374 N-cyclopropyl-N-(5-(4-(cyclopropylamino)quinazolin-6-yl)-2-fluorobenzyl)-3-fluorobenzamide,
375 N-cyclopropyl-N-(5-(4-(cyclopropylamino)quinazolin-6-yl)-2-fluorobenzyl)-3-(trifluoromethyl)benzole sulfonamide,
376 N-cyclopropyl-N-(3-(4-(phenethylamino)quinazolin-6-yl)benzyl)benzamide,
377 N-cyclopropyl-N-(3-(4-(phenethylamino)quinazolin-6-yl)benzyl)isonicotinamide,
378 N-cyclopropyl-N-(3-(4-(3-methylbenzylamino)quinazolin-6-yl)benzyl)thiophene-2-sulfonamide,
379 N-cyclopropyl-N-(3-(4-(3-methylbenzylamino)quinazolin-6-yl)benzyl)-2-(trifluoromethyl)benzamide,
380 N-cyclopropyl-N-(3-(4-(3-methylbenzylamino)quinazolin-6-yl)benzyl)methane sulfonamide,
381 N-cyclopropyl-N-(5-(4-(cyclopropylamino)quinazolin-6-yl)-2-fluorobenzyl)-2,4-difluorobenzamide,
382 N-cyclopropyl-N-(5-(4-(cyclopropylamino)quinazolin-6-yl)-2-fluorobenzyl)pentanamide,
383 N-cyclopropyl-N-(5-(4-(cyclopropylamino)quinazolin-6-yl)-2-fluorobenzyl)-4-methoxybenzole sulfonamide
384 N-cyclopropyl-3,4-dimethoxy-N-(3-(4-(phenethylamino)quinazolin-6-yl)benzyl)benzamide,
385 N-cyclopropyl-N-(3-(4-(phenethylamino)quinazolin-6-yl)benzyl)nicotinamide,
386 N-cyclopropyl-N-(3-(4-(3-methylbenzylamino)quinazolin-6-yl)benzyl)furan-2-carboxamide,
387 3,5-dichloro-N-cyclopropyl-N-(3-(4-(3-methylbenzylamino)quinazolin-6-yl)benzyl)benzamide,
388 N-cyclopropyl-N-(3-(4-(3-methylbenzylamino)quinazolin-6-yl)benzyl)-1-phenylmethane sulfonamide,
389 N-cyclopropyl-N-(5-(4-(cyclopropylamino)quinazolin-6-yl)-2-fluorobenzyl)-2,3,4,5,6-pentafluorobenzamide,
390 N-cyclopropyl-N-(5-(4-(cyclopropylamino)quinazolin-6-yl)-2-fluorobenzyl)-2-methoxyacetamide,
391 2,4-dichloro-N-cyclopropyl-N-(5-(4-(cyclopropylamino)quinazolin-6-yl)-2-fluorobenzyl)benzole sulfonamide,
392 N-cyclopropyl-3-fluoro-N-(3-(4-(phenethylamino)quinazolin-6-yl)benzyl)benzamide,
393 N-cyclopropyl-N-(3-(4-(phenethylamino)quinazolin-6-yl)benzyl)acetamide,
394 N-cyclopropyl-N-(3-(4-(3-methylbenzylamino)quinazolin-6-yl)benzyl)thiophene-2-carboxamide,
395 N-cyclopropyl-N-(3-(4-(3-methylbenzylamino)quinazolin-6-yl)benzyl)picolinamide,
396 2,4-dichloro-N-cyclopropyl-N-(3-(4-(3-methylbenzylamino)quinazolin-6-yl)benzyl)benzole sulfonamide,
397 N-cyclopropyl-N-(5-(4-(cyclopropylamino)quinazolin-6-yl)-2-fluorobenzyl)-2-fluoro-4-(trifluoromethyl)benzamide,
398 N-cyclopropyl-N-(5-(4-(cyclopropylamino)quinazolin-6-yl)-2-fluorobenzyl)-2-(dimethylamino)acetamide,
399 N-cyclopropyl-N-(5-(4-(cyclopropylamino)quinazolin-6-yl)-2-fluorobenzyl)-2,3,4,5,6-pentafluorobenzole sulfonamide,
400 N-cyclopropyl-2,4-difluoro-N-(3-(4-(phenethylamino)quinazolin-6-yl)benzyl)benzamide,
401 N-cyclopropyl-N-(3-(4-(phenethylamino)quinazolin-6-yl)benzyl)pivalamide,
402 N-cyclopropyl-1,3-dimethyl-N-(3-(4-(3-methylbenzylamino)quinazolin-6-yl)benzyl)-1H-pyrazole-5-carboxamide,
403 N-cyclopropyl-N-(3-(4-(3-methylbenzylamino)quinazolin-6-yl)benzyl)isonicotinamide,
404 N-cyclopropyl-2,3,4,5,6-pentafluoro-N-(3-(4-(3-methylbenzylamino)quinazolin-6-yl)benzyl)benzole sulfonamide,
405 N-cyclopropyl-N-(5-(4-(cyclopropylamino)quinazolin-6-yl)-2-fluorobenzyl)-3-(trifluoromethyl)benzamide,
406 N-cyclopropyl-N-(5-(4-(cyclopropylamino)quinazolin-6-yl)-2-fluorobenzyl)-2-phenylacetamide,
407 N-cyclopropyl-N-(5-(4-(cyclopropylamino)quinazolin-6-yl)-2-fluorobenzyl)thiophene-2-sulfonamide,
408 N-cyclopropyl-2,3,4,5,6-pentafluoro-N-(3-(4-(phenethylamino)quinazolin-6-yl)benzyl)benzamide,
409 N-cyclopropyl-N-(3-(4-(phenethylamino)quinazolin-6-yl)benzyl)pentanamide,
410 N-cyclopropyl-N-(3-(4-(3-methylbenzylamino)quinazolin-6-yl)benzyl)benzamide,
411 N-cyclopropyl-N-(3-(4-(3-methylbenzylamino)quinazolin-6-yl)benzyl)nicotinamide,
412 N-cyclopropyl-N-(3-(4-(3-methylbenzylamino)quinazolin-6-yl)benzyl)quinoline-8-sulfonamide,
413 N-cyclopropyl-N-(5-(4-(cyclopropylamino)quinazolin-6-yl)-2-fluorobenzyl)-2-(trifluoromethyl)benzamide,
414 N-cyclopropyl-N-(5-(4-(cyclopropylamino)quinazolin-6-yl)-2-fluorobenzyl)-3-phenylpropanamide,
415 N-cyclopropyl-N-(5-(4-(cyclopropylamino)quinazolin-6-yl)-2-fluorobenzyl)quinoline-8-sulfonamide,
416 N-cyclopropyl-2-fluoro-N-(3-(4-(phenethylamino)quinazolin-6-yl)benzyl)-4-(trifluoromethyl)benzamide,
417 N-cyclopropyl-2-(dimethylamino)-N-(3-(4-(phenethylamino)quinazolin-6-yl)benzyl)acetamide,
418 N-cyclopropyl-3,4-dimethoxy-N-(3-(4-(3-methylbenzylamino)quinazolin-6-yl)benzyl)benzamide,
419 N-cyclopropyl-N-(3-(4-(3-methylbenzylamino)quinazolin-6-yl)benzyl)acetamide,
420 N-cyclopropyl-N-(5-(4-(cyclopropyl(methyl)amino)quinazolin-6-yl)-2-fluorobenzyl)furan-2-carboxamide,
421 3,5-dichloro-N-cyclopropyl-N-(5-(4-(cyclopropylamino)quinazolin-6-yl)-2-fluorobenzyl)benzamide,
422 N-cyclopropyl-N-(3-(4-(phenethylamino)quinazolin-6-yl)benzyl)furan-2-carboxamide,
423 N-cyclopropyl-N-(3-(4-(phenethylamino)quinazolin-6-yl)benzyl)-3-(trifluoromethyl)benzamide,
424 N-cyclopropyl-N-(3-(4-(phenethylamino)quinazolin-6-yl)benzyl)-2-phenylacetamide,
425 N-cyclopropyl-3-fluoro-N-(3-(4-(3-methylbenzylamino)quinazolin-6-yl)benzyl)benzamide,
426 N-cyclopropyl-N-(3-(4-(3-methylbenzylamino)quinazolin-6-yl)benzyl)pentanamide,
427 N-cyclopropyl-N-(5-(4-(cyclopropyl(methyl)amino)quinazolin-6-yl)-2-fluorobenzyl)thiophene-2-carboxamide,
428 N-cyclopropyl-N-(5-(4-(cyclopropylamino)quinazolin-6-yl)-2-fluorobenzyl)picolinamide
429 N-cyclopropyl-N-(5-(4-(cyclopropylamino)quinazolin-6-yl)-2-fluorobenzyl)propane-2-sulfonamide,
430 N-cyclopropyl-N-(3-(4-(phenethylamino)quinazolin-6-yl)benzyl)thiophene-2-carboxamide,
431 N-cyclopropyl-N-(3-(4-(phenethylamino)quinazolin-6-yl)benzyl)-2-(trifluoromethyl)benzamide,
432 N-cyclopropyl-N-(3-(4-(phenethylamino)quinazolin-6-yl)benzyl)-3-phenylpropanamide,
433 N-cyclopropyl-2,4-difluoro-N-(3-(4-(3-methylbenzylamino)quinazolin-6-yl)benzyl)benzamide,
434 N-cyclopropyl-2-methoxy-N-(3-(4-(3-methylbenzylamino)quinazolin-6-yl)benzyl)acetamide,
435 N-cyclopropyl-N-(5-(4-(cyclopropylamino)quinazolin-6-yl)-2-fluorobenzyl)-1,3-dimethyl-1H-pyrazole-5-carboxamide,
436 N-cyclopropyl-N-(5-(4-(cyclopropylamino)quinazolin-6-yl)-2-fluorobenzyl)isonicotinamide,
437 N-cyclopropyl-N-(5-(4-(cyclopropylamino)quinazolin-6-yl)-2-fluorobenzyl)-1-phenylmethane sulfonamide,
438 N-cyclopropyl-N-(3-(4-(phenethylamino)quinazolin-6-yl)benzyl)isoxazole-5-carboxamide,
439 3,5-dichloro-N-cyclopropyl-N-(3-(4-(phenethylamino)quinazolin-6-yl)benzyl)benzamide,
440 N-cyclopropyl-N-(3-(4-(phenethylamino)quinazolin-6-yl)benzyl)methane sulfonamide,
441 N-cyclopropyl-N-(3-(4-(phenethylamino)quinazolin-6-yl)benzyl)-1-phenylmethane sulfonamide,
442 N-cyclopropyl-2,3,4,5,6-pentafluoro-N-(3-(4-(3-methylbenzylamino)quinazolin-6-yl)benzyl)benzamide,
443 2,3,4,5,6-pentafluoro-N-(3-(4-(methylamino)quinazolin-6-yl)benzyl)-N-(pyridin-2-ylmethyl)benzole sulfonamide,
444 N-(3-(4-(methylamino)quinazolin-6-yl)benzyl)-N-(pyridin-2-ylmethyl)-3-(trifluoromethyl)benzamide,
445 N-(3-(4-(methylamino)quinazolin-6-yl)benzyl)-3-phenyl-N-(pyridin-2-ylmethyl)propanamide,
446 N-(5-(4-(benzylamino)quinazolin-6-yl)-2-fluorobenzyl)-2,4-dichloro-N-methylbenzole sulfonamide,
447 N-cyclopropyl-N-(3-(4-(cyclopropylamino)-2-methylquinazolin-6-yl)benzyl)-2,3,4,5,6-pentafluorobenzamide,
448 2,4-dichloro-N-cyclopropyl-N-(3-(4-(cyclopropylamino)-2-methylquinazolin-6-yl)benzyl)benzole sulfonamide,
449 N-cyclopropyl-N-(3-(4-(phenethylamino)quinazolin-6-yl)benzyl)benzole sulfonamide,
450 N-(3-(4-(methylamino)quinazolin-6-yl)benzyl)-N-(pyridin-2-ylmethyl)quinoline-8-sulfonamide,
451 3,5-dichloro-N-(3-(4-(methylamino)quinazolin-6-yl)benzyl)-N-(pyridin-2-ylmethyl)benzamide,
452 N-cyclopropyl-N-(5-(4-(cyclopropylamino)quinazolin-6-yl)-2-fluorobenzyl)cyclohexane carboxamide,
453 N-(5-(4-(benzylamino)quinazolin-6-yl)-2-fluorobenzyl)-2,3,4,5,6-pentafluoro-N-methylbenzole sulfonamide,
454 N-cyclopropyl-N-(3-(4-(cyclopropylamino)-2-methylquinazolin-6-yl)benzyl)-2-fluoro-4-(trifluoromethyl)benzamide,
455 N-cyclopropyl-N-(3-(4-(cyclopropylamino)-2-methylquinazolin-6-yl)benzyl)-2,3,4,5,6-pentafluorobenzole sulfonamide,
456 N-cyclopropyl-N-(3-(4-(phenethylamino)quinazolin-6-yl)benzyl)-3-(trifluoromethyl)benzole sulfonamide,
457 N-cyclopropyl-N-(5-(4-(cyclopropylamino)quinazolin-6-yl)-2-fluorobenzyl)isoxazole-5-carboxamide,
458 N-(3-(4-(methylamino)quinazolin-6-yl)benzyl)-N-(pyridin-2-ylmethyl)furan-2-carboxamide,
459 N-(3-(4-(methylamino)quinazolin-6-yl)benzyl)-N-(pyridin-2-ylmethyl)picolinamide,
460 N-cyclopropyl-N-(3-(4-(phenethylamino)quinazolin-6-yl)benzyl)cyclohexane carboxamide,
461 N-(5-(4-(benzylamino)quinazolin-6-yl)-2-fluorobenzyl)-N-methylthiophene-2-sulfonamide,
462 N-cyclopropyl-N-(3-(4-(cyclopropylamino)-2-methylquinazolin-6-yl)benzyl)-3-(trifluoromethyl)benzamide,
463 N-cyclopropyl-N-(3-(4-(cyclopropylamino)-2-methylquinazolin-6-yl)benzyl)thiophene-2-sulfonamide,
464 N-cyclopropyl-4-methoxy-N-(3-(4-(phenethylamino)quinazolin-6-yl)benzyl)benzole sulfonamide,
465 N-cyclopropyl-N-(3-(4-(3-methylbenzylamino)quinazolin-6-yl)benzyl)pivalamide,
466 N-(3-(4-(methylamino)quinazolin-6-yl)benzyl)-N-(pyridin-2-ylmethyl)thiophene-2-carboxamide,
467 N-(3-(4-(methylamino)quinazolin-6-yl)benzyl)-N-(pyridin-2-ylmethyl)isonicotinamide,
468 N-cyclopropyl-N-(3-(4-(3-methylbenzylamino)quinazolin-6-yl)benzyl)cyclohexane carboxamide,
469 N-(5-(4-(benzylamino)quinazolin-6-yl)-2-fluorobenzyl)-N-methylquinoline-8-sulfonamide,
470 N-cyclopropyl-N-(5-(4-(cyclopropylamino)-2-methylquinazolin-6-yl)-2-fluorobenzyl)-2-(trifluoromethyl)benzamide,
471 N-cyclopropyl-N-(3-(4-(cyclopropylamino)-2-methylquinazolin-6-yl)benzyl)quinoline-8-sulfonamide,
472 2,4-dichloro-N-cyclopropyl-N-(3-(4-(phenethylamino)quinazolin-6-yl)benzyl)benzole sulfonamide,
473 N-cyclopropyl-N-(3-(4-(3-methylbenzylamino)quinazolin-6-yl)benzyl)-3-(trifluoromethyl)benzole sulfonamide,
474 1,3-dimethyl-N-(3-(4-(methylamino)quinazolin-6-yl)benzyl)-N-(pyridin-2-ylmethyl)-1H-pyrazole-5-carboxamide,
475 N-(3-(4-(methylamino)quinazolin-6-yl)benzyl)-N-(pyridin-2-ylmethyl)nicotinamide,
476 N-(5-(4-(benzylamino)quinazolin-6-yl)-2-fluorobenzyl)-N-methylmethane sulfonamide,
477 N-cyclopropyl-N-(3-(4-(cyclopropylamino)-2-methylquinazolin-6-yl)benzyl)isoxazole-5-carboxamide,
478 N-cyclopropyl-N-(3-(4-(cyclopropylamino)-2-methylquinazolin-6-yl)benzyl)methane sulfonamide,
479 3,5-dichloro-N-cyclopropyl-N-(3-(4-(cyclopropylamino)-2-methylquinazolin-6-yl)benzyl)benzamide,
480 N-cyclopropyl-2,3,4,5,6-pentafluoro-N-(3-(4-(phenethylamino)quinazolin-6-yl)benzyl)benzole sulfonamide,
481 N-(3-(4-(methylamino)quinazolin-6-yl)benzyl)-N-(pyridin-2-ylmethyl)methane sulfonamide,
482 N-(3-(4-(methylamino)quinazolin-6-yl)benzyl)-N-(pyridin-2-ylmethyl)benzamide,
483 N-(3-(4-(methylamino)quinazolin-6-yl)benzyl)-N-(pyridin-2-ylmethyl)acetamide,
484 N-(5-(4-(benzylamino)quinazolin-6-yl)-2-fluorobenzyl)-N-methylpropane-2-sulfonamide,
485 N-cyclopropyl-N-(3-(4-(cyclopropylamino)-2-methylquinazolin-6-yl)benzyl)-1,3-dimethyl-1H-pyrazole-5-carboxamide,
486 N-cyclopropyl-N-(3-(4-(cyclopropylamino)-2-methylquinazolin-6-yl)benzyl)propane-2-sulfonamide,
487 N-cyclopropyl-N-(3-(4-(cyclopropylamino)-2-methylquinazolin-6-yl)benzyl)picolinamide,
488 N-cyclopropyl-N-(3-(4-(phenethylamino)quinazolin-6-yl)benzyl)thiophene-2-sulfonamide,
489 N-(3-(4-(methylamino)quinazolin-6-yl)benzyl)-1-phenyl-N-(pyridin-2-ylmethyl)methane sulfonamide,
490 3,4-dimethoxy-N-(3-(4-(methylamino)quinazolin-6-yl)benzyl)-N-(pyridin-2-ylmethyl)benzamide,
491 N-(3-(4-(methylamino)quinazolin-6-yl)benzyl)-N-(pyridin-2-ylmethyl)pentanamide,
492 N-(5-(4-(benzylamino)quinazolin-6-yl)-2-fluorobenzyl)-N-methyl-1-phenylmethane sulfonamide,
493 N-cyclopropyl-N-(3-(4-(cyclopropylamino)-2-methylquinazolin-6-yl)benzyl)benzamide,
494 N-cyclopropyl-N-(3-(4-(cyclopropylamino)-2-methylquinazolin-6-yl)benzyl)-1-phenylmethane sulfonamide,
495 N-cyclopropyl-N-(3-(4-(cyclopropylamino)-2-methylquinazolin-6-yl)benzyl)isonicotinamide,
496 N-cyclopropyl-N-(3-(4-(phenethylamino)quinazolin-6-yl)benzyl)quinoline-8-sulfonamide,
497 N-(3-(4-(methylamino)quinazolin-6-yl)benzyl)-N-(pyridin-2-ylmethyl)benzole sulfonamide,
498 3-fluoro-N-(3-(4-(methylamino)quinazolin-6-yl)benzyl)-N-(pyridin-2-ylmethyl)benzamide,
499 2-methoxy-N-(3-(4-(methylamino)quinazolin-6-yl)benzyl)-N-(pyridin-2-ylmethyl)acetamide,
500 N-(5-(4-(benzylamino)quinazolin-6-yl)-2-fluorobenzyl)-N-methylbenzole sulfonamide,
501 N-cyclopropyl-N-(3-(4-(cyclopropylamino)-2-methylquinazolin-6-yl)benzyl)-3,4-dimethoxybenzamide,
502 N-cyclopropyl-N-(3-(4-(cyclopropylamino)-2-methylquinazolin-6-yl)benzyl)benzole sulfonamide,
503 N-cyclopropyl-N-(3-(4-(cyclopropylamino)-2-methylquinazolin-6-yl)benzyl)nicotinamide,
504 N-cyclopropyl-N-(3-(4-(3-methylbenzylamino)quinazolin-6-yl)benzyl)propane-2-sulfonamide,
505 N-(3-(4-(methylamino)quinazolin-6-yl)benzyl)-N-(pyridin-2-ylmethyl)-3-(trifluoromethyl)benzole sulfonamide,
506 2,4-difluoro-N-(3-(4-(methylamino)quinazolin-6-yl)benzyl)-N-(pyridin-2-ylmethyl)benzamide,
507 N-(3-(4-(methylamino)quinazolin-6-yl)benzyl)-N-(pyridin-2-ylmethyl)cyclohexane carboxamide,
508 N-(5-(4-(benzylamino)quinazolin-6-yl)-2-fluorobenzyl)-N-methyl-3-(trifluoromethyl)benzole sulfonamide,
509 N-cyclopropyl-N-(3-(4-(cyclopropylamino)-2-methylquinazolin-6-yl)benzyl)-3-fluorobenzamide,
510 N-cyclopropyl-N-(3-(4-(cyclopropylamino)-2-methylquinazolin-6-yl)benzyl)-3-(trifluoromethyl)benzole sulfonamide,
511 N-cyclopropyl-N-(3-(4-(cyclopropylamino)-2-methylquinazolin-6-yl)benzyl)acetamide,
512 N-cyclopropyl-N-(3-(4-(3-methylbenzylamino)quinazolin-6-yl)benzyl)isoxazole-5-carboxamide,
513 4-methoxy-N-(3-(4-(methylamino)quinazolin-6-yl)benzyl)-N-(pyridin-2-ylmethyl)benzole sulfonamide,
514 2-fluoro-N-(3-(4-(methylamino)quinazolin-6-yl)benzyl)-N-(pyridin-2-ylmethyl)-4-(trifluoromethyl)benzamide,
515 N-(3-(4-(methylamino)quinazolin-6-yl)benzyl)-2-phenyl-N-(pyridin-2-ylmethyl)acetamide,
516 N-(5-(4-(benzylamino)quinazolin-6-yl)-2-fluorobenzyl)-4-methoxy-N-methylbenzole sulfonamide,
517 N-cyclopropyl-N-(3-(4-(cyclopropylamino)-2-methylquinazolin-6-yl)benzyl)-2,4-difluorobenzamide,
518 N-cyclopropyl-N-(3-(4-(cyclopropylamino)-2-methylquinazolin-6-yl)benzyl)-4-methoxybenzole sulfonamide,
519 N-cyclopropyl-N-(3-(4-(cyclopropylamino)-2-methylquinazolin-6-yl)benzyl)pentanamide,
520 N-cyclopropyl-N-(3-(4-(cyclopropylamino)-2-methylquinazoline-6-yl)benzyl)-2-methoxyacetamide,
521 N-(2-fluoro-5-(4-(methylamino)quinazolin-6-yl)benzyl)-N-methylfuran-2-carboxamide,
522 N-(2-fluoro-5-(4-(methylamino)quinazolin-6-yl)benzyl)-N-methyl-3-(trifluoromethyl)benzamide,
523 N-(2-fluoro-5-(4-(methylamino)quinazolin-6-yl)benzyl)-N-methyl-3-(trifluoromethyl)benzole sulfonamide,
524 N-cyclopropyl-N-(5-(4-(cyclopropylamino)-2-methylquinazolin-6-yl)-2-fluorobenzyl)-2,4-difluorobenzamide,
525 N-(5-(4-(benzylamino)quinazolin-6-yl)-2-fluorobenzyl)-N-methyl-2-(trifluoromethyl)benzamide,
526 N-cyclopropyl-N-(5-(4-(cyclopropylamino)-2-methylquinazolin-6-yl)-2-fluorobenzyl)furan-2-carboxamide,
527 N-cyclopropyl-N-(5-(4-(cyclopropylamino)-2-methylquinazolin-6-yl)-2-fluorobenzyl)methane sulfonamide,
528 N-cyclopropyl-N-(3-(4-(cyclopropylamino)-2-methylquinazolin-6-yl)benzyl)-2-(dimethylamino)acetamide,
529 N-(2-fluoro-5-(4-(methylamino)quinazolin-6-yl)benzyl)-N-methylthiophene-2-carboxamide,
530 N-(2-fluoro-5-(4-(methylamino)quinazolin-6-yl)benzyl)-N-methyl-2-(trifluoromethyl)benzamide,
531 2,4-dichloro-N-(2-fluoro-5-(4-(methylamino)quinazolin-6-yl)benzyl)-N-methylbenzole sulfonamide,
532 N-cyclopropyl-N-(5-(4-(cyclopropylamino)-2-methylquinazolin-6-yl)-2-fluorobenzyl)-2-fluoro-4-(trifluoromethyl)benzamide,
533 N-(5-(4-(benzylamino)quinazolin-6-yl)-2-fluorobenzyl)-3,5-dichloro-N-methylbenzamide,
534 N-cyclopropyl-N-(5-(4-(cyclopropylamino)-2-methylquinazolin-6-yl)-2-fluorobenzyl)thiophene-2-carboxamide,
535 N-cyclopropyl-N-(5-(4-(cyclopropylamino)-2-methylquinazolin-6-yl)-2-fluorobenzyl)propane-2-sulfonamide,
536 N-cyclopropyl-N-(3-(4-(cyclopropylamino)-2-methylquinazolin-6-yl)benzyl)cyclohexane carboxamide,
537 N-(2-fluoro-5-(4-(methylamino)quinazolin-6-yl)benzyl)-N-methylisoxazole-5-carboxamide,
538 N-(2-fluoro-5-(4-(methylamino)quinazolin-6-yl)benzyl)-N-methylacetamide,
539 2,3,4,5,6-pentafluoro-N-(2-fluoro-5-(4-(methylamino)quinazolin-6-yl)benzyl)-N-methylbenzole sulfonamide,
540 N-cyclopropyl-N-(5-(4-(cyclopropylamino)-2-methylquinazolin-6-yl)-2-fluorobenzyl)pentanamide,
541 N-(5-(4-(benzylamino)quinazolin-6-yl)-2-fluorobenzyl)-N-methyl pivalamide,
542 N-cyclopropyl-N-(5-(4-(cyclopropylamino)-2-methylquinazolin-6-yl)-2-fluorobenzyl)-1,3-dimethyl-1H-pyrazole-5-carboxamide,
543 N-cyclopropyl-N-(5-(4-(cyclopropylamino)-2-methylquinazolin-6-yl)-2-fluorobenzyl)-1-phenylmethane sulfonamide,
544 N-cyclopropyl-N-(3-(4-(cyclopropylamino)-2-methylquinazolin-6-yl)benzyl)-2-phenylacetamide,
545 N-(2-fluoro-5-(4-(methylamino)quinazolin-6-yl)benzyl)-N,1,3-trimethyl-1H-pyrazole-5-carboxamide,
546 N-(2-fluoro-5-(4-(methylamino)quinazolin-6-yl)benzyl)-N-methyl cyclohexane carboxamide,
547 N-(2-fluoro-5-(4-(methylamino)quinazolin-6-yl)benzyl)-N-methylthiophene-2-sulfonamide,
548 N-(5-(4-(benzylamino)quinazolin-6-yl)-2-fluorobenzyl)-N,1,3-trimethyl-1H-pyrazole-5-carboxamide,
549 N-(5-(4-(benzylamino)quinazolin-6-yl)-2-fluorobenzyl)-N-methyl pentanamide,
550 N-cyclopropyl-N-(5-(4-(cyclopropylamino)-2-methylquinazolin-6-yl)-2-fluorobenzyl)benzamide,
551 N-cyclopropyl-N-(5-(4-(cyclopropylamino)-2-methylquinazolin-6-yl)-2-fluorobenzyl)benzole sulfonamide,
552 N-cyclopropyl-N-(3-(4-(cyclopropylamino)-2-methylquinazolin-6-yl)benzyl)-3-phenylpropanamide,
553 N-(2-fluoro-5-(4-(methylamino)quinazolin-6-yl)benzyl)-N-methylbenzamide,
554 N-(2-fluoro-5-(4-(methylamino)quinazolin-6-yl)benzyl)-2-methoxy-N-methylacetamide,
555 N-(2-fluoro-5-(4-(methylamino)quinazolin-6-yl)benzyl)-N-methylquinoline-8-sulfonamide,
556 N-(5-(4-(benzylamino)quinazolin-6-yl)-2-fluorobenzyl)-N-methylbenzamide,
557 N-(5-(4-(benzylamino)quinazolin-6-yl)-2-fluorobenzyl)-2-methoxy-N-methylacetamide,
558 N-cyclopropyl-N-(5-(4-(cyclopropylamino)-2-methylquinazolin-6-yl)-2-fluorobenzyl)-3,4-dimethoxybenzamide,
559 N-cyclopropyl-N-(5-(4-(cyclopropylamino)-2-methylquinazolin-6-yl)-2-fluorobenzyl)-3-(trifluoromethyl)benzole sulfonamide,
560 N-(2-fluoro-5-(4-(methylamino)quinazolin-6-yl)benzyl)-4-methoxy-N-methylbenzole sulfonamide,
561 N-(2-fluoro-5-(4-(methylamino)quinazolin-6-yl)benzyl)-3,4-dimethoxy-N-methylbenzamide,
562 N-(2-fluoro-5-(4-(methylamino)quinazolin-6-yl)benzyl)-N-methyl-3-phenylpropanamide,
563 N-(5-(4-(benzylamino)quinazolin-6-yl)-2-fluorobenzyl)-N-methylthiophene-2-carboxamide,
564 N-(5-(4-(benzylamino)quinazolin-6-yl)-2-fluorobenzyl)-3,4-dimethoxy-N-methylbenzamide,
565 N-(5-(4-(benzylamino)quinazolin-6-yl)-2-fluorobenzyl)-2-(dimethylamino)-N-methylacetamide,
566 N-cyclopropyl-N-(5-(4-(cyclopropylamino)-2-methylquinazolin-6-yl)-2-fluorobenzyl)-3-fluorobenzamide,
567 N-cyclopropyl-N-(5-(4-(cyclopropylamino)-2-methylquinazolin-6-yl)-2-fluorobenzyl)-4-methoxybenzole sulfonamide,
568 N-(2-fluoro-5-(4-(methylamino)quinazolin-6-yl)benzyl)-N-methylisonicotinamide,
569 3-fluoro-N-(2-fluoro-5-(4-(methylamino)quinazolin-6-yl)benzyl)-N-methylbenzamide,
570 N-(2-fluoro-5-(4-(methylamino)quinazolin-6-yl)benzyl)-N-methylmethane sulfonamide,
571 N-(5-(4-(benzylamino)quinazolin-6-yl)-2-fluorobenzyl)-3-fluoro-N-methylbenzamide,
572 N-(5-(4-(benzylamino)quinazolin-6-yl)-2-fluorobenzyl)-2,4-difluoro-N-methylbenzamide,
573 N-(2-fluoro-5-(4-(methylamino)quinazolin-6-yl)benzyl)-N-methyl nicotinamide,
574 N-cyclopropyl-N-(5-(4-(cyclopropylamino)-2-methylquinazolin-6-yl)-2-fluorobenzyl)-2,3,4,5,6-pentafluorobenzamide,
575 2,4-dichloro-N-cyclopropyl-N-(5-(4-(cyclopropylamino)-2-methylquinazolin-6-yl)-2-fluorobenzyl)benzole sulfonamide
576 N-(2-fluoro-5-(4-(methylamino)quinazolin-6-yl)benzyl)-N-methylpentanamide,
577 2,4-difluoro-N-(2-fluoro-5-(4-(methylamino)quinazolin-6-yl)benzyl)-N-methylbenzamide,
578 N-(2-fluoro-5-(4-(methylamino)quinazolin-6-yl)benzyl)-N-methylpropane-2-sulfonamide,
579 N-(5-(4-(benzylamino)quinazolin-6-yl)-2-fluorobenzyl)-N-methyl isonicotinamide,
580 N-(5-(4-(benzylamino)quinazolin-6-yl)-2-fluorobenzyl)-2,3,4,5,6-pentafluoro-N-methylbenzamide,
581 N-(5-(4-(benzylamino)quinazolin-6-yl)-2-fluorobenzyl)-N-methyl cyclohexane carboxamide,
582 N-cyclopropyl-N-(5-(4-(cyclopropylamino)-2-methylquinazolin-6-yl)-2-fluorobenzyl)-3-(trifluoromethyl)benzamide,
583 N-cyclopropyl-N-(5-(4-(cyclopropylamino)-2-methylquinazolin-6-yl)-2-fluorobenzyl)-2,3,4,5,6-pentafluorobenzole sulfonamide,
584 N-(2-fluoro-5-(4-(methylamino)quinazolin-6-yl)benzyl)-N-methyl-2-phenylacetamide,
585 2,3,4,5,6-pentafluoro-N-(2-fluoro-5-(4-(methylamino)quinazolin-6-yl)benzyl)-N-methylbenzamide,
586 N-(2-fluoro-5-(4-(methylamino)quinazolin-6-yl)benzyl)-N-methyl-1-phenylmethane sulfonamide,
587 N-(5-(4-(benzylamino)quinazolin-6-yl)-2-fluorobenzyl)-N-methyl nicotinamide,
588 N-(5-(4-(benzylamino)quinazolin-6-yl)-2-fluorobenzyl)-2-fluoro-N-methyl-4-(trifluoromethyl)benzamide,
589 N-(5-(4-(benzylamino)quinazolin-6-yl)-2-fluorobenzyl)-N-methyl-2-phenylacetamide,
590 N-(2-fluoro-5-(4-(methylamino)quinazolin-6-yl)benzyl)-N-methyl pivalamide,
591 N-cyclopropyl-N-(5-(4-(cyclopropylamino)-2-methylquinazolin-6-yl)-2-fluorobenzyl)thiophene-2-sulfonamide,
592 N-(2-fluoro-5-(4-(methylamino)quinazolin-6-yl)benzyl)-N-methyl picolinamide,
593 2-fluoro-N-(2-fluoro-5-(4-(methylamino)quinazolin-6-yl)benzyl)-N-methyl-4-(trifluoromethyl)benzamide,
594 N-(2-fluoro-5-(4-(methylamino)quinazolin-6-yl)benzyl)-N-methyl benzole sulfonamide,
595 N-(5-(4-(benzylamino)quinazolin-6-yl)-2-fluorobenzyl)-N-methylacetamide,
596 N-(5-(4-(benzylamino)quinazolin-6-yl)-2-fluorobenzyl)-N-methyl-3-(trifluoromethyl)benzamide,
597 N-(5-(4-(benzylamino)quinazolin-6-yl)-2-fluorobenzyl)-N-methyl-3-phenylpropanamide,
598 N-(5-(4-(benzylamino)quinazolin-6-yl)-2-fluorobenzyl)-N-methylfuran-2-carboxamide,
599 N-cyclopropyl-N-(5-(4-(cyclopropylamino)-2-methylquinazolin-6-yl)-2-fluorobenzyl)quinoline-8-sulfonamide,
600 3,5-dichloro-N-cyclopropyl-N-(5-(4-(cyclopropylamino)-2-methylquinazolin-6-yl)-2-fluorobenzyl)benzamide,
601 N-cyclopropyl-N-(5-(4-(cyclopropylamino)-2-methylquinazolin-6-yl)-2-fluorobenzyl)-3-phenylpropanamide,
602 N-cyclopropyl-N-(3-(4-(2,4-dimethoxybenzylamino)quinazolin-6-yl)benzyl)benzole sulfonamide,
603 N-cyclopropyl-N-(3-(4-(2,4-dimethoxybenzylamino)quinazolin-6-yl)benzyl)isonicotinamide,
604 N-cyclopropyl-N-(5-(4-(cyclopropylamino)-2-methylquinazolin-6-yl)-2-fluorobenzyl)picolinamide,
605 N-cyclopropyl-N-(5-(4-(cyclopropylamino)quinazolin-6-yl)-2-fluorobenzyl)-2-methylthiazole-4-carboxamide,
606 N-cyclopropyl-N-(3-(4-(2,4-dimethoxybenzylamino)quinazolin-6-yl)benzyl)-3-(trifluoromethyl)benzole sulfonamide,
607 N-cyclopropyl-N-(3-(4-(2,4-dimethoxybenzylamino)quinazolin-6-yl)benzyl)benzamide,
608 N-cyclopropyl-N-(5-(4-(cyclopropylamino)-2-methylquinazolin-6-yl)-2-fluorobenzyl)isonicotinamide,
609 N-cyclopropyl-2-methyl-N-(3-(4-(phenethylamino)quinazolin-6-yl)benzyl)thiazole-4-carboxamide,
610 N-cyclopropyl-N-(3-(4-(2,4-dimethoxybenzylamino)quinazolin-6-yl)benzyl)-4-methoxybenzole sulfonamide,
611 N-cyclopropyl-N-(3-(4-(2,4-dimethoxybenzylamino)quinazolin-6-yl)benzyl)-3-fluorobenzamide,
612 N-cyclopropyl-N-(5-(4-(cyclopropylamino)-2-methylquinazolin-6-yl)-2-fluorobenzyl)nicotinamide,
613 N-cyclopropyl-N-(3-(4-(2,4-dimethoxybenzylamino)quinazolin-6-yl)benzyl)-2-methylthiazole-4-carboxamide,
614 2,4-dichloro-N-cyclopropyl-N-(3-(4-(2,4-dimethoxybenzylamino)quinazolin-6-yl)benzyl)benzole sulfonamide,
615 N-cyclopropyl-N-(3-(4-(2,4-dimethoxybenzylamino)quinazolin-6-yl)benzyl)-2,4-difluorobenzamide,
616 N-cyclopropyl-N-(5-(4-(cyclopropylamino)-2-methylquinazolin-6-yl)-2-fluorobenzyl)acetamide,
617 N-(2-fluoro-5-(4-(methylamino)quinazolin-6-yl)benzyl)-N,2-dimethylthiazole-4-carboxamide,
618 N-cyclopropyl-2-methyl-N-(3-(4-(3-methylbenzylamino)quinazolin-6-yl)benzyl)thiazole-4-carboxamide,
619 N-cyclopropyl-N-(3-(4-(cyclopropylamino)-8-fluoroquinazolin-6-yl)benzyl)-2,4-difluorobenzamide,
620 N-cyclopropyl-N-(3-(4-(2,4-dimethoxybenzylamino)quinazolin-6-yl)benzyl)-2,3,4,5,6-pentafluorobenzamide,
621 N-cyclopropyl-N-(5-(4-(cyclopropylamino)-2-methylquinazolin-6-yl)-2-fluorobenzyl)pivalamide,
622 N-(5-(4-(benzylamino)quinazolin-6-yl)-2-fluorobenzyl)-N,2-dimethylthiazole-4-carboxamide,
623 2,4-dichloro-N-(3-(4-(methylamino)quinazolin-6-yl)benzyl)-N-(pyridin-2-ylmethyl)benzole sulfonamide,
624 N-cyclopropyl-N-(5-(4-(cyclopropylamino)-2-methylquinazolin-6-yl)-2-fluorobenzyl)-2-methylthiazole-4-carboxamide,
625 N-cyclopropyl-N-(3-(4-(2,4-dimethoxybenzylamino)quinazolin-6-yl)benzyl)thiophene-2-sulfonamide,
626 N-cyclopropyl-N-(3-(4-(2,4-dimethoxybenzylamino)quinazolin-6-yl)benzyl)-2-fluoro-4-(trifluoromethyl)benzamide,
627 N-cyclopropyl-N-(5-(4-(cyclopropylamino)-2-methylquinazolin-6-yl)-2-fluorobenzyl)-2-methoxyacetamide,
628 N-(3-(4-(methylamino)quinazolin-6-yl)benzyl)-N-(pyridin-2-ylmethyl)pivalamide,
629 N-(3-(4-(methylamino)quinazolin-6-yl)benzyl)-N-(pyridin-2-ylmethyl)thiophene-2-sulfonamide,
630 2-methyl-N-(3-(4-(methylamino)quinazolin-6-yl)benzyl)-N-(pyridin-2-ylmethyl)thiazole-4-carboxamide,
631 N-cyclopropyl-N-(3-(4-(2,4-dimethoxybenzylamino)quinazolin-6-yl)benzyl)quinoline-8-sulfonamide,
632 N-cyclopropyl-N-(3-(4-(2,4-dimethoxybenzylamino)quinazolin-6-yl)benzyl)-3-(trifluoromethyl)benzamide,
633 N-cyclopropyl-N-(5-(4-(cyclopropylamino)-2-methylquinazolin-6-yl)-2-fluorobenzyl)-2-(dimethylamino)acetamide,
634 N-cyclopropyl-N-(3-(4-(cyclopropylamino)-2-methylquinazolin-6-yl)benzyl)-2-methylthiazole-4-carboxamide,
635 N-(3-(4-(methylamino)quinazolin-6-yl)benzyl)-N-(pyridin-2-ylmethyl)propane-2-sulfonamide,
636 N-(5-(4-(benzylamino)quinazolin-6-yl)-2-fluorobenzyl)-N-methyl picolinamide,
637 N-cyclopropyl-N-(3-(4-(2,4-dimethoxybenzylamino)quinazolin-6-yl)benzyl)furan-2-carboxamide,
638 N-cyclopropyl-N-(3-(4-(2,4-dimethoxybenzylamino)quinazolin-6-yl)benzyl)-2-(trifluoromethyl)benzamide,
639 N-cyclopropyl-N-(5-(4-(cyclopropylamino)-2-methylquinazolin-6-yl)-2-fluorobenzyl)cyclohexane carboxamide,
640 N-cyclopropyl-N-(3-(4-(cyclopropylamino)-2-methylquinazolin-6-yl)benzyl)pivalamide,
641 N-cyclopropyl-N-(3-(4-(2,4-dimethoxybenzylamino)quinazolin-6-yl)benzyl)methane sulfonamide,
642 N-cyclopropyl-N-(3-(4-(2,4-dimethoxybenzylamino)quinazolin-6-yl)benzyl)thiophene-2-carboxamide,
643 3,5-dichloro-N-cyclopropyl-N-(3-(4-(2,4-dimethoxybenzylamino)quinazolin-6-yl)benzyl)benzamide,
644 N-cyclopropyl-N-(5-(4-(cyclopropylamino)-2-methylquinazolin-6-yl)-2-fluorobenzyl)-2-phenylacetamide,
645 N-cyclopropyl-N-(5-(4-(cyclopropylamino)quinazolin-6-yl)-2-fluorobenzyl) pivalamide,
646 N-cyclopropyl-N-(3-(4-(2,4-dimethoxybenzylamino)quinazolin-6-yl)benzyl)-1-phenylmethane sulfonamide,
647 N-cyclopropyl-N-(3-(4-(2,4-dimethoxybenzylamino)quinazolin-6-yl)benzyl) isoxazole-5-carboxamide,
648 N-cyclopropyl-N-(3-(4-(2,4-dimethoxybenzylamino)quinazolin-6-yl)benzyl) picolinamide,
649 N-cyclopropyl-N-(3-(4-(2,4-dimethoxybenzylamino)quinazolin-6-yl)benzyl)nicotinamide,
650 N-cyclopropyl-N-(3-(4-(cyclopropylamino)-2-methylquinazolin-6-yl)benzyl)furan-2-carboxamide,
651 N-cyclopropyl-N-(3-(4-(cyclopropylamino)-7-methylquinazolin-6-yl)benzyl)-3-(trifluoromethyl)benzole sulfonamide,
652 N-cyclopropyl-N-(3-(4-(cyclopropylamino)-7-methylquinazolin-6-yl)benzyl)-3-fluorobenzamide,
653 N-cyclopropyl-N-(3-(4-(cyclopropylamino)-7-methylquinazolin-6-yl)benzyl)cyclohexane carboxamide,
654 N-cyclopropyl-N-(3-(4-(cyclopropylamino)-8-fluoroquinazolin-6-yl)benzyl)pentanamide,
655 N-(2-fluorophenyl)-N-(3-(4-(methylamino)quinazolin-6-yl)benzyl)thiophene-2-sulfonamide,
656 N-cyclopropyl-N-(3-(4-(2,4-dimethoxybenzylamino)quinazolin-6-yl)benzyl)-3,4-dimethoxybenzamide,
657 N-cyclopropyl-N-(3-(4-(2,4-dimethoxybenzylamino)quinazolin-6-yl)benzyl)acetamide,
658 N-cyclopropyl-N-(3-(4-(cyclopropylamino)-2-methylquinazolin-6-yl)benzyl)thiophene-2-carboxamide,
659 N-cyclopropyl-N-(3-(4-(cyclopropylamino)-7-methylquinazolin-6-yl)benzyl)-4-methoxybenzole sulfonamide,
660 N-cyclopropyl-N-(3-(4-(cyclopropylamino)-7-methylquinazolin-6-yl)benzyl)-2,4-difluorobenzamide,
661 N-cyclopropyl-N-(3-(4-(cyclopropylamino)-7-methylquinazolin-6-yl)benzyl)-2-phenylacetamide,
662 N-cyclopropyl-N-(3-(4-(cyclopropylamino)-8-fluoroquinazolin-6-yl)benzyl)cyclohexane carboxamide,
663 N-(2-fluorophenyl)-N-(3-(4-(methylamino)quinazolin-6-yl)benzyl)quinoline-8-sulfonamide,
664 N-(5-(4-(benzylamino)quinazolin-6-yl)-2-fluorobenzyl)-N-methylisoxazole-5-carboxamide,
665 N-cyclopropyl-N-(3-(4-(2,4-dimethoxybenzylamino)quinazolin-6-yl)benzyl)pivalamide,
666 N-cyclopropyl-N-(3-(4-(cyclopropylamino)-2-methylquinazolin-6-yl)benzyl)-2-(trifluoromethyl)benzamide,
667 2,4-dichloro-N-cyclopropyl-N-(3-(4-(cyclopropylamino)-7-methylquinazolin-6-yl)benzyl)benzole sulfonamide,
668 N-cyclopropyl-N-(3-(4-(cyclopropylamino)-7-methylquinazolin-6-yl)benzyl)-2-fluoro-4-(trifluoromethyl)benzamide,
669 N-cyclopropyl-N-(3-(4-(cyclopropylamino)-7-methylquinazolin-6-yl)benzyl)-3-phenylpropanamide,
670 N-cyclopropyl-N-(3-(4-(cyclopropylamino)-8-fluoroquinazolin-6-yl)benzyl)-2-phenylacetamide,
671 N-(2-fluorophenyl)-N-(3-(4-(methylamino)quinazolin-6-yl)benzyl)isoxazole-5-carboxamide,
672 N-(2-fluorophenyl)-N-(3-(4-(methylamino)quinazolin-6-yl)benzyl)-3-(trifluoromethyl)benzole sulfonamide,
673 N-cyclopropyl-N-(3-(4-(2,4-dimethoxybenzylamino)quinazolin-6-yl)benzyl)pentanamide,
674 3,5-dichloro-N-(2-fluoro-5-(4-(methylamino)quinazolin-6-yl)benzyl)-N-methylbenzamide,
675 N-cyclopropyl-N-(3-(4-(cyclopropylamino)-7-methylquinazolin-6-yl)benzyl)-2,3,4,5,6-pentafluorobenzole sulfonamide,
676 N-cyclopropyl-N-(3-(4-(cyclopropylamino)-7-methylquinazolin-6-yl)benzyl)-3-(trifluoromethyl)benzamide,
677 N-cyclopropyl-N-(3-(4-(cyclopropylamino)-8-fluoroquinazolin-6-yl)benzyl)furan-2-carboxamide,
678 N-cyclopropyl-N-(3-(4-(cyclopropylamino)-8-fluoroquinazolin-6-yl)benzyl)methane sulfonamide,
679 N-(2-fluorophenyl)-N-(3-(4-(methylamino)quinazolin-6-yl)benzyl)benzamide,
680 N-(2-fluorophenyl)-4-methoxy-N-(3-(4-(methylamino)quinazolin-6-yl)benzyl)benzole sulfonamide,
681 N-cyclopropyl-N-(3-(4-(2,4-dimethoxybenzylamino)quinazolin-6-yl)benzyl)-2-methoxyacetamide,
682 N-cyclopropyl-N-(5-(4-(cyclopropylamino)-2-methylquinazolin-6-yl)-2-fluorobenzyl)isoxazole-5-carboxamide,
683 N-cyclopropyl-N-(3-(4-(cyclopropylamino)-7-methylquinazolin-6-yl)benzyl)quinoline-8-sulfonamide,
684 N-cyclopropyl-N-(3-(4-(cyclopropylamino)-7-methylquinazolin-6-yl)benzyl)-2-(trifluoromethyl)benzamide,
685 N-cyclopropyl-N-(3-(4-(cyclopropylamino)-8-fluoroquinazolin-6-yl)benzyl)thiophene-2-carboxamide,
686 N-cyclopropyl-N-(3-(4-(cyclopropylamino)-8-fluoroquinazolin-6-yl)benzyl)-3-(trifluoromethyl)benzole sulfonamide,
687 3-fluoro-N-(2-fluorophenyl)-N-(3-(4-(methylamino)quinazolin-6-yl)benzyl)benzamide,
688 N-(3-(4-(methylamino)quinazolin-6-yl)benzyl)-N-(pyridin-2-ylmethyl)isoxazole-5-carboxamide,
689 N-cyclopropyl-N-(3-(4-(2,4-dimethoxybenzylamino)quinazolin-6-yl)benzyl)-2-(dimethylamino)acetamide,
690 N-cyclopropyl-N-(3-(4-(cyclopropylamino)-8-fluoroquinazolin-6-yl)benzyl)benzole sulfonamide,
691 N-cyclopropyl-N-(3-(4-(cyclopropylamino)-7-methylquinazolin-6-yl)benzyl)furan-2-carboxamide,
692 3,5-dichloro-N-cyclopropyl-N-(3-(4-(cyclopropylamino)-7-methylquinazolin-6-yl)benzyl)benzamide,
693 N-cyclopropyl-N-(3-(4-(cyclopropylamino)-8-fluoroquinazolin-6-yl)benzyl)benzamide,
694 N-cyclopropyl-N-(3-(4-(cyclopropylamino)-8-fluoroquinazolin-6-yl)benzyl)-4-methoxybenzole sulfonamide,
695 2,3,4,5,6-pentafluoro-N-(2-fluorophenyl)-N-(3-(4-(methylamino)quinazolin-6-yl)benzyl)benzamide,
696 N-cyclopropyl-N-(3-(4-(2,4-dimethoxybenzylamino)quinazolin-6-yl)benzyl)cyclohexane carboxamide,
697 N-cyclopropyl-N-(3-(4-(cyclopropylamino)-7-methylquinazolin-6-yl)benzyl)methane sulfonamide,
698 N-cyclopropyl-N-(3-(4-(cyclopropylamino)-7-methylquinazolin-6-yl)benzyl)thiophene-2-carboxamide,
699 N-cyclopropyl-N-(3-(4-(cyclopropylamino)-7-methylquinazolin-6-yl)benzyl)picolinamide,
700 N-cyclopropyl-N-(3-(4-(cyclopropylamino)-8-fluoroquinazolin-6-yl)benzyl)-3,4-dimethoxybenzamide,
701 N-cyclopropyl-N-(3-(4-(cyclopropylamino)-8-fluoroquinazolin-6-yl)benzyl)thiophene-2-sulfonamide,
702 N-cyclopropyl-N-(3-(4-(cyclopropylamino)-7-methylquinazolin-6-yl)benzyl)nicotinamide,
703 N-cyclopropyl-N-(3-(4-(cyclopropylamino)-8-fluoroquinazolin-6-yl)benzyl)-3-phenylpropanamide,
704 N-cyclopropyl-N-(3-(4-(2,4-dimethoxybenzylamino)quinazolin-6-yl)benzyl)-2-phenylacetamide,
705 N-cyclopropyl-N-(3-(4-(cyclopropylamino)-7-methylquinazolin-6-yl)benzyl)propane-2-sulfonamide,
706 N-cyclopropyl-N-(3-(4-(cyclopropylamino)-7-methylquinazolin-6-yl)benzyl)-1,3-dimethyl-1H-pyrazole-5-carboxamide,
707 N-cyclopropyl-N-(3-(4-(cyclopropylamino)-7-methylquinazolin-6-yl)benzyl)isonicotinamide,
708 N-cyclopropyl-N-(3-(4-(cyclopropylamino)-8-fluoroquinazolin-6-yl)benzyl)picolinamide,
709 N-cyclopropyl-N-(3-(4-(cyclopropylamino)-8-fluoroquinazolin-6-yl)benzyl)quinoline-8-sulfonamide,
710 N-cyclopropyl-N-(3-(4-(cyclopropylamino)-7-methylquinazolin-6-yl)benzyl)acetamide,
711 N-(2-fluorophenyl)-N-(3-(4-(methylamino)quinazolin-6-yl)benzyl)acetamide,
712 N-cyclopropyl-N-(3-(4-(2,4-dimethoxybenzylamino)quinazolin-6-yl)benzyl)-3-phenylpropanamide,
713 N-cyclopropyl-N-(3-(4-(cyclopropylamino)-7-methylquinazolin-6-yl)benzyl)-1-phenylmethane sulfonamide,
714 N-cyclopropyl-N-(3-(4-(cyclopropylamino)-7-methylquinazolin-6-yl)benzyl)benzamide,
715 N-cyclopropyl-N-(3-(4-(cyclopropylamino)-7-methylquinazolin-6-yl)benzyl)pentanamide,
716 N-cyclopropyl-N-(3-(4-(cyclopropylamino)-8-fluoroquinazolin-6-yl)benzyl)acetamide,
717 N-(2-fluorophenyl)-N-(3-(4-(methylamino)quinazolin-6-yl)benzyl)methane sulfonamide,
718 2-(dimethylamino)-N-(2-fluoro-5-(4-(methylamino)quinazolin-6-yl)benzyl)-N-methylacetamide,
719 N-cyclopropyl-N-(3-(4-(cyclopropylamino)-7-methylquinazolin-6-yl)benzyl)pivalamide,
720 N-(3-(4-(methylamino)quinazolin-6-yl)benzyl)-N-(pyridin-2-ylmethyl)-2-(trifluoromethyl)benzamide,
721 N-cyclopropyl-N-(3-(4-(cyclopropylamino)-7-methylquinazolin-6-yl)benzyl)benzole sulfonamide,
722 N-cyclopropyl-N-(3-(4-(cyclopropylamino)-7-methylquinazolin-6-yl)benzyl)-3,4-dimethoxybenzamide,
723 N-cyclopropyl-N-(3-(4-(cyclopropylamino)-7-methylquinazolin-6-yl)benzyl)-2-(dimethylamino)acetamide,
724 N-cyclopropyl-N-(3-(4-(cyclopropylamino)-8-fluoroquinazolin-6-yl)benzyl)pivalamide,
725 2,4-dichloro-N-(2-fluorophenyl)-N-(3-(4-(methylamino)quinazolin-6-yl)benzyl)benzole sulfonamide,
726 N-cyclopropyl-2-(dimethylamino)-N-(3-(4-(3-methylbenzylamino)quinazolin-6-yl)benzyl)acetamide,
727 N-cyclopropyl-N-(3-(4-(cyclopropylamino)-8-fluoroquinazolin-6-yl)benzyl)-3-fluorobenzamide,
728 N-cyclopropyl-N-(3-(4-(cyclopropylamino)-8-fluoroquinazolin-6-yl)benzyl)-3-(trifluoromethyl)benzamide,
729 3,5-dichloro-N-(2-fluorophenyl)-N-(3-(4-(methylamino)quinazolin-6-yl)benzyl)benzamide,
730 N-cyclopropyl-N-(3-(4-(2,4-dimethoxybenzylamino)quinazolin-6-yl)benzyl)-1,3-dimethyl-1H-pyrazole-5-carboxamide,
731 N-cyclopropyl-N-((4-(4-(cyclopropylamino)quinazolin-6-yl)pyridin-2-yl)methyl)benzole sulfonamide,
732 N-(2-chloro-5-(4-(cyclopropylamino)quinazolin-6-yl)benzyl)-N-cyclopropyl-3-(trifluoromethyl)benzamide,
733 N-(2-chloro-5-(4-(cyclopropylamino)quinazolin-6-yl)benzyl)-N-cyclopropyl-2-(dimethylamino)acetamide,
734 N-(2-chloro-5-(4-(cyclopropylamino)quinazolin-6-yl)benzyl)-N-cyclopropylthiophene-2-sulfonamide,
735 N-cyclopropyl-N-((4-(4-(cyclopropylamino)quinazolin-6-yl)pyridin-2-yl)methyl)-2-fluoro-4-(trifluoromethyl)benzamide,
736 N-cyclopropyl-N-(3-(4-(cyclopropylamino)-8-fluoroquinazolin-6-yl)benzyl)isonicotinamide,
737 N-cyclopropyl-N-(3-(4-(cyclopropylamino)-7-methylquinazolin-6-yl)benzyl)thiophene-2-sulfonamide,
738 N-cyclopropyl-N-(3-(4-(cyclopropylamino)-8-fluoroquinazolin-6-yl)benzyl)-2-fluoro-4-(trifluoromethyl)benzamide,
739 N-cyclopropyl-N-((4-(4-(cyclopropylamino)quinazolin-6-yl)pyridin-2-yl)methyl)-2-(dimethylamino)acetamide,
740 N-(2-chloro-5-(4-(cyclopropylamino)quinazolin-6-yl)benzyl)-N-cyclopropyl-2-(trifluoromethyl)benzamide,
741 N-(2-chloro-5-(4-(cyclopropylamino)quinazolin-6-yl)benzyl)-N-cyclopropylcyclohexane carboxamide,
742 N-(2-chloro-5-(4-(cyclopropylamino)quinazolin-6-yl)benzyl)-N-cyclopropylquinoline-8-sulfonamide,
743 N-cyclopropyl-N-((4-(4-(cyclopropylamino)quinazolin-6-yl)pyridin-2-yl)methyl)-3-(trifluoromethyl)benzamide,
744 N-cyclopropyl-N-(3-(4-(cyclopropylamino)-8-fluoroquinazolin-6-yl)benzyl)-2-methoxyacetamide,
745 N-cyclopropyl-N-(3-(4-(cyclopropylamino)-7-methylquinazolin-6-yl)benzyl)isoxazole-5-carboxamide,
746 N-(2-chloro-5-(4-(cyclopropylamino)quinazolin-6-yl)benzyl)-N-cyclopropylfuran-2-carboxamide,
747 3,5-dichloro-N-(2-chloro-5-(4-(cyclopropylamino)quinazolin-6-yl)benzyl)-N-cyclopropylbenzamide,
748 N-(2-chloro-5-(4-(cyclopropylamino)quinazolin-6-yl)benzyl)-N-cyclopropyl-2-phenylacetamide,
749 N-cyclopropyl-N-((4-(4-(cyclopropylamino)quinazolin-6-yl)pyridin-2-yl)methyl)-2-(trifluoromethyl)benzamide,
750 3,5-dichloro-N-cyclopropyl-N-((4-(4-(cyclopropylamino)quinazolin-6-yl)pyridin-2-yl)methyl)benzamide,
751 N-cyclopropyl-N-(3-(4-(cyclopropylamino)-8-fluoroquinazolin-6-yl)benzyl)-2-(dimethylamino)acetamide,
752 N-cyclopropyl-N-(3-(4-(cyclopropylamino)-8-fluoroquinazolin-6-yl)benzyl)-2,3,4,5,6-pentafluorobenzamide,
753 N-cyclopropyl-N-(3-(4-(cyclopropylamino)-8-fluoroquinazolin-6-yl)benzyl)-1-phenylmethane sulfonamide,
754 N-(2-chloro-5-(4-(cyclopropylamino)quinazolin-6-yl)benzyl)-N-cyclopropylthiophene-2-carboxamide,
755 N-(2-chloro-5-(4-(cyclopropylamino)quinazolin-6-yl)benzyl)-N-cyclopropylpicolinamide,
756 N-(2-chloro-5-(4-(cyclopropylamino)quinazolin-6-yl)benzyl)-N-cyclopropyl-3-phenylpropanamide,
757 N-cyclopropyl-N-((4-(4-(cyclopropylamino)quinazolin-6-yl)pyridin-2-yl)methyl)furan-2-carboxamide,
758 N-cyclopropyl-N-((4-(4-(cyclopropylamino)quinazolin-6-yl)pyridin-2-yl)methyl)picolinamide,
759 N-cyclopropyl-N-(3-(4-(cyclopropylamino)-7-methylquinazolin-6-yl)benzyl)-2-methylthiazole-4-carboxamide,
760 N-cyclopropyl-N-(3-(4-(cyclopropylamino)-8-fluoroquinazolin-6-yl)benzyl)-1,3-dimethyl-1H-pyrazole-5-carboxamide,
761 N-cyclopropyl-N-(3-(4-(cyclopropylamino)-8-fluoroquinazolin-6-yl)benzyl)-2,3,4,5,6-pentafluorobenzole sulfonamide,
762 N-(2-chloro-5-(4-(cyclopropylamino)quinazolin-6-yl)benzyl)-N-cyclopropyl isoxazole-5-carboxamide,
763 N-(2-chloro-5-(4-(cyclopropylamino)quinazolin-6-yl)benzyl)-N-cyclopropyl isonicotinamide,
764 N-(2-chloro-5-(4-(cyclopropylamino)quinazolin-6-yl)benzyl)-N-cyclopropyl methane sulfonamide,
765 N-cyclopropyl-N-((4-(4-(cyclopropylamino)quinazolin-6-yl)pyridin-2-yl)methyl)isoxazole-5-carboxamide,
766 N-cyclopropyl-N-((4-(4-(cyclopropylamino)quinazolin-6-yl)pyridin-2-yl)methyl)nicotinamide,
767 N-(2-fluorophenyl)-N-(3-(4-(methylamino)quinazolin-6-yl)benzyl)furan-2-carboxamide,
768 N-cyclopropyl-N-(3-(4-(cyclopropylamino)-7-methylquinazolin-6-yl)benzyl)-2,3,4,5,6-pentafluorobenzamide,
769 N-(2-chloro-5-(4-(cyclopropylamino)quinazolin-6-yl)benzyl)-N-cyclopropyl-2-methylthiazole-4-carboxamide,
770 N-(2-chloro-5-(4-(cyclopropylamino)quinazolin-6-yl)benzyl)-N-cyclopropyl nicotinamide,
771 N-(2-chloro-5-(4-(cyclopropylamino)quinazolin-6-yl)benzyl)-N-cyclopropyl-1-phenylmethane sulfonamide,
772 N-cyclopropyl-N-((4-(4-(cyclopropylamino)quinazolin-6-yl)pyridin-2-yl)methyl)-2-methylthiazole-4-carboxamide,
773 N-cyclopropyl-N-((4-(4-(cyclopropylamino)quinazolin-6-yl)pyridin-2-yl)methyl)acetamide,
774 3,5-dichloro-N-cyclopropyl-N-(3-(4-(cyclopropylamino)-8-fluoroquinazolin-6-yl)benzyl)benzamide,
775 N-cyclopropyl-N-(3-(4-(cyclopropylamino)-8-fluoroquinazolin-6-yl)benzyl)isoxazole-5-carboxamide,
776 N-(2-chloro-5-(4-(cyclopropylamino)quinazolin-6-yl)benzyl)-N-cyclopropyl-1,3-dimethyl-1H-pyrazole-5-carboxamide,
777 N-(2-chloro-5-(4-(cyclopropylamino)quinazolin-6-yl)benzyl)-N-cyclopropyl acetamide,
778 N-(2-chloro-5-(4-(cyclopropylamino)quinazolin-6-yl)benzyl)-N-cyclopropyl-3-(trifluoromethyl)benzole sulfonamide,
779 N-cyclopropyl-N-((4-(4-(cyclopropylamino)quinazolin-6-yl)pyridin-2-yl)methyl)-1,3-dimethyl-1H-pyrazole-5-carboxamide,
780 N-cyclopropyl-N-((4-(4-(cyclopropylamino)quinazolin-6-yl)pyridin-2-yl)methyl)-2-methoxyacetamide,
781 N-cyclopropyl-N-(3-(4-(cyclopropylamino)-8-fluoroquinazolin-6-yl)benzyl)nicotinamide,
782 2,4-dichloro-N-cyclopropyl-N-(3-(4-(cyclopropylamino)-8-fluoroquinazolin-6-yl)benzyl)benzole sulfonamide,
783 N-(2-chloro-5-(4-(cyclopropylamino)quinazolin-6-yl)benzyl)-N-cyclopropyl benzamide,
784 N-(2-chloro-5-(4-(cyclopropylamino)quinazolin-6-yl)benzyl)-N-cyclopropyl-3,4-dimethoxybenzamide,
785 N-(2-chloro-5-(4-(cyclopropylamino)quinazolin-6-yl)benzyl)-N-cyclopropyl pivalamide,
786 N-(2-chloro-5-(4-(cyclopropylamino)quinazolin-6-yl)benzyl)-N-cyclopropyl-4-methoxybenzole sulfonamide,
787 N-cyclopropyl-N-((4-(4-(cyclopropylamino)quinazolin-6-yl)pyridin-2-yl)methyl)-3,4-dimethoxybenzamide,
788 N-cyclopropyl-N-((4-(4-(cyclopropylamino)quinazolin-6-yl)pyridin-2-yl)methyl)thiophene-2-sulfonamide,
789 N-cyclopropyl-N-(3-(4-(cyclopropylamino)-8-fluoroquinazolin-6-yl)benzyl)-2-(trifluoromethyl)benzamide,
790 2-(dimethylamino)-N-(3-(4-(methylamino)quinazolin-6-yl)benzyl)-N-(pyridin-2-ylmethyl)acetamide,
791 N-(2-chloro-5-(4-(cyclopropylamino)quinazolin-6-yl)benzyl)-N-cyclopropyl-3-fluorobenzamide,
792 N-(2-chloro-5-(4-(cyclopropylamino)quinazolin-6-yl)benzyl)-N-cyclopropyl-2,4-difluorobenzamide,
793 N-(2-chloro-5-(4-(cyclopropylamino)quinazolin-6-yl)benzyl)-N-cyclopropyl pentanamide,
794 2,4-dichloro-N-(2-chloro-5-(4-(cyclopropylamino)quinazolin-6-yl)benzyl)-N-cyclopropyl benzole sulfonamide,
795 N-cyclopropyl-N-((4-(4-(cyclopropylamino)quinazolin-6-yl)pyridin-2-yl)methyl)-3-fluorobenzamide,
796 2,4-dichloro-N-cyclopropyl-N-((4-(4-(cyclopropylamino)quinazolin-6-yl)pyridin-2-yl)methyl)benzole sulfonamide,
797 N-cyclopropyl-N-(3-(4-(cyclopropylamino)-7-methylquinazolin-6-yl)benzyl)-2-methoxyacetamide,
798 N-cyclopropyl-2-methoxy-N-(3-(4-(phenethylamino)quinazolin-6-yl)benzyl)acetamide,
799 N-(2-chloro-5-(4-(cyclopropylamino)quinazolin-6-yl)benzyl)-N-cyclopropyl benzole sulfonamide,
800 N-(2-chloro-5-(4-(cyclopropylamino)quinazolin-6-yl)benzyl)-N-cyclopropyl-2-fluoro-4-(trifluoromethyl)benzamide,
801 N-(2-chloro-5-(4-(cyclopropylamino)quinazolin-6-yl)benzyl)-N-cyclopropyl-2-methoxyacetamide,
802 N-(2-chloro-5-(4-(cyclopropylamino)quinazolin-6-yl)benzyl)-N-cyclopropyl-2,3,4,5,6-pentafluorobenzole sulfonamide,
803 N-cyclopropyl-N-((4-(4-(cyclopropylamino)quinazolin-6-yl)pyridin-2-yl)methyl)-2,3,4,5,6-pentafluorobenzamide,
804 N-cyclopropyl-N-((4-(4-(cyclopropylamino)quinazolin-6-yl)pyridin-2-yl)methyl)propane-2-sulfonamide,
805 N-cyclopropyl-N-((4-(4-(cyclopropylamino)quinazolin-6-yl)pyridin-2-yl)methyl)-1-phenylmethane sulfonamide,
806 N-cyclopropyl-N-((4-(4-(cyclopropylamino)quinazolin-6-yl)pyridin-2-yl)methyl)-4-methoxybenzole sulfonamide,
807 N-(5-(4-(tert-butylamino)quinazolin-6-yl)-2-fluorobenzyl)-N-cyclopropyl furan-2-carboxamide,
808 N-(5-(4-(tert-butylamino)quinazolin-6-yl)-2-fluorobenzyl)-N-cyclopropyl-3-(trifluoromethyl)benzamide,
809 N-(5-(4-(tert-butylamino)quinazolin-6-yl)-2-fluorobenzyl)-N-cyclopropyl cyclohexane carboxamide,
810 N-(5-(4-(tert-butylamino)quinazolin-6-yl)-2-fluorobenzyl)-N-cyclopropyl thiophene-2-sulfonamide,
811 N-cyclopropyl-N-((4-(4-(cyclopropylamino)quinazolin-6-yl)pyridin-2-yl)methyl)thiophene-2-carboxamide,
812 N-cyclopropyl-N-((4-(4-(cyclopropylamino)quinazolin-6-yl)pyridin-2-yl)methyl)quinoline-8-sulfonamide,
813 N-cyclopropyl-N-((4-(4-(cyclopropylamino)quinazolin-6-yl)pyridin-2-yl)methyl)benzamide,
814 N-(5-(4-(tert-butylamino)quinazolin-6-yl)-2-fluorobenzyl)-N-cyclopropyl-2-(trifluoromethyl)benzamide,
815 N-(5-(4-(tert-butylamino)quinazolin-6-yl)-2-fluorobenzyl)-N-cyclopropyl-2-phenylacetamide,
816 N-(5-(4-(tert-butylamino)quinazolin-6-yl)-2-fluorobenzyl)-N-cyclopropyl quinoline-8-sulfonamide,
817 N-cyclopropyl-N-((4-(4-(cyclopropylamino)quinazolin-6-yl)pyridin-2-yl)methyl)-2,4-difluorobenzamide,
818 N-cyclopropyl-N-(3-(4-(phenethylamino)quinazolin-6-yl)benzyl)propane-2-sulfonamide,
819 N-(5-(4-(tert-butylamino)quinazolin-6-yl)-2-fluorobenzyl)-N-cyclopropyl thiophene-2-carboxamide,
820 N-(5-(4-(tert-butylamino)quinazolin-6-yl)-2-fluorobenzyl)-3,5-dichloro-N-cyclopropyl benzamide,
821 N-(5-(4-(tert-butylamino)quinazolin-6-yl)-2-fluorobenzyl)-N-cyclopropyl-3-phenyl propanamide,
822 N-(5-(4-(tert-butylamino)quinazolin-6-yl)-2-fluorobenzyl)-N-cyclopropyl propane-2-sulfonamide,
823 N-cyclopropyl-N-((4-(4-(cyclopropylamino)quinazolin-6-yl)pyridin-2-yl)methyl)isonicotinamide,
824 N-cyclopropyl-N-(3-(4-(cyclopropylamino)-8-fluoroquinazolin-6-yl)benzyl)propane-2-sulfonamide,
825 N-(5-(4-(tert-butylamino)quinazolin-6-yl)-2-fluorobenzyl)-N-cyclopropyl-2-methylthiazole-4-carboxamide,
826 N-(5-(4-(tert-butylamino)quinazolin-6-yl)-2-fluorobenzyl)-N-cyclopropyl picolinamide,
827 N-(5-(4-(tert-butylamino)quinazolin-6-yl)-2-fluorobenzyl)-N-cyclopropyl methane sulfonamide,
828 N-cyclopropyl-N-((4-(4-(cyclopropylamino)quinazolin-6-yl)pyridin-2-yl)methyl)pivalamide,
829 N-cyclopropyl-N-((4-(4-(cyclopropylamino)quinazolin-6-yl)pyridin-2-yl)methyl)pentanamide,
830 N-cyclopropyl-N-(3-(4-(cyclopropylamino)-8-fluoroquinazolin-6-yl)benzyl)-2-methylthiazole-4-carboxamide,
831 N-(5-(4-(tert-butylamino)quinazolin-6-yl)-2-fluorobenzyl)-N-cyclopropyl-1,3-dimethyl-1H-pyrazole-5-carboxamide,
832 N-(5-(4-(tert-butylamino)quinazolin-6-yl)-2-fluorobenzyl)-N-cyclopropyl isonicotinamide,
833 N-(5-(4-(tert-butylamino)quinazolin-6-yl)-2-fluorobenzyl)-N-cyclopropyl-1-phenyl methane sulfonamide,
834 N-(5-(4-(tert-butylamino)quinazolin-6-yl)-2-fluorobenzyl)-N-cyclopropyl isoxazole-5-carboxamide,
835 N-cyclopropyl-N-((4-(4-(cyclopropylamino)quinazolin-6-yl)pyridin-2-yl)methyl)cyclohexane carboxamide,
836 N-cyclopropyl-N-(3-(4-(2,4-dimethoxybenzylamino)quinazolin-6-yl)benzyl)propane-2-sulfonamide,
837 N-(5-(4-(tert-butylamino)quinazolin-6-yl)-2-fluorobenzyl)-N-cyclopropyl benzamide,
838 N-(5-(4-(tert-butylamino)quinazolin-6-yl)-2-fluorobenzyl)-N-cyclopropyl nicotinamide,
839 N-(5-(4-(tert-butylamino)quinazolin-6-yl)-2-fluorobenzyl)-N-cyclopropyl benzole sulfonamide,
840 N-(5-(4-(tert-butylamino)quinazolin-6-yl)-2-fluorobenzyl)-N-cyclopropyl-2,3,4,5,6-pentafluorobenzamide,
841 N-cyclopropyl-N-((4-(4-(cyclopropylamino)quinazolin-6-yl)pyridin-2-yl)methyl)-2-phenylacetamide,
842 N-cyclopropyl-N-(3-(4-(2,4-dimethoxybenzylamino)quinazolin-6-yl)benzyl)-2,3,4,5,6-pentafluorobenzole sulfonamide,
843 N-(5-(4-(tert-butylamino)quinazolin-6-yl)-2-fluorobenzyl)-N-cyclopropyl-3,4-dimethoxybenzamide,
844 N-(5-(4-(tert-butylamino)quinazolin-6-yl)-2-fluorobenzyl)-N-cyclopropyl acetamide,
845 N-(5-(4-(tert-butylamino)quinazolin-6-yl)-2-fluorobenzyl)-N-cyclopropyl-3-(trifluoromethyl)benzole sulfonamide,
846 N-(5-(4-(tert-butylamino)quinazolin-6-yl)-2-fluorobenzyl)-N-cyclopropyl-2-methoxyacetamide,
847 N-cyclopropyl-N-((4-(4-(cyclopropylamino)quinazolin-6-yl)pyridin-2-yl)methyl)-3-phenylpropanamide,
848 N-cyclopropyl-N-((4-(4-(cyclopropylamino)quinazolin-6-yl)pyridin-2-yl)methyl)-2,3,4,5,6-pentafluorobenzole sulfonamide,
849 N-(5-(4-(tert-butylamino)quinazolin-6-yl)-2-fluorobenzyl)-N-cyclopropyl-3-fluorobenzamide,
850 N-(5-(4-(tert-butylamino)quinazolin-6-yl)-2-fluorobenzyl)-N-cyclopropyl pivalamide,
851 N-(5-(4-(tert-butylamino)quinazolin-6-yl)-2-fluorobenzyl)-N-cyclopropyl-4-methoxybenzole sulfonamide,
852 N-(2-fluorophenyl)-N-(3-(4-(methylamino)quinazolin-6-yl)benzyl)benzole sulfonamide,
853 N-cyclopropyl-N-((4-(4-(cyclopropylamino)quinazolin-6-yl)pyridin-2-yl)methyl)methane sulfonamide,
854 N-(2-chloro-5-(4-(cyclopropylamino)quinazolin-6-yl)benzyl)-N-cyclopropyl propane-2-sulfonamide,
855 N-(5-(4-(tert-butylamino)quinazolin-6-yl)-2-fluorobenzyl)-N-cyclopropyl-2,4-difluorobenzamide,
856 N-(5-(4-(tert-butylamino)quinazolin-6-yl)-2-fluorobenzyl)-N-cyclopropyl pentanamide,
857 N-(5-(4-(tert-butylamino)quinazolin-6-yl)-2-fluorobenzyl)-2,4-dichloro-N-cyclopropyl benzole sulfonamide,
858 N-cyclopropyl-N-((4-(4-(cyclopropylamino)quinazolin-6-yl)pyridin-2-yl)methyl)-3-(trifluoromethyl)benzole sulfonamide,
859 N-(2-chloro-5-(4-(cyclopropylamino)quinazolin-6-yl)benzyl)-N-cyclopropyl-2,3,4,5,6-pentafluorobenzamide,
860 N-(5-(4-(tert-butylamino)quinazolin-6-yl)-2-fluorobenzyl)-N-cyclopropyl-2-fluoro-4-(trifluoromethyl)benzamide,
861 N-(5-(4-(tert-butylamino)quinazolin-6-yl)-2-fluorobenzyl)-N-cyclopropyl-2-(dimethylamino)acetamide,
862 N-(5-(4-(tert-butylamino)quinazolin-6-yl)-2-fluorobenzyl)-N-cyclopropyl-2,3,4,5,6-pentafluorobenzole sulfonamide,
866 1-(3-(4-(neopentylamino)quinazolin-6-yl)benzyl)pyrrolidine-2,5-dione,
867 1-(3-(4-(2-methoxyethylamino)quinazolin-6-yl)benzyl)pyrrolidine-2,5-dione hydrochloride,
868 N-cyclopropyl-N-(3-(4-(cyclopropylamino)quinazolin-6-yl)benzyl)-1-methylpiperidine-4-carboxamide,
869 1-amino-N-(3-(4-(cyclopropylamino)quinazolin-6-yl)benzyl)cyclopropane carboxamide dihydrochloride,
870 N-cyclopropyl-N-(5-(4-(cyclopropylamino)quinazolin-6-yl)-2-fluorobenzyl)-2,2,2-trifluoroacetamide,
871 1-(3-(4-morpholinoquinazolin-6-yl)benzyl)pyrrolidine-2,5-dione,
872 1-(3-(4-(cyclobutylamino)quinazolin-6-yl)benzyl)pyrrolidine-2,5-dione hydrochloride,
873 1-(3-(4-(isopropylamino)quinazolin-6-yl)benzyl)pyrrolidine-2,5-dione hydrochloride,
874 N-(3-(4-(cyclopropylamino)quinazolin-6-yl)benzyl)-1-pentanamidocyclopropane carboxamide,
875 ethyl cyclopropyl(3-(4-(cyclopropylamino)quinazolin-6-yl)benzyl)carbamate,
876 6-(3-((1H-pyrrol-1-yl)methyl)phenyl)-N-cyclopropylquinazoline-4-amine,
877 1-(3-(4-(cyclopropylamino)quinazolin-6-yl)benzyl)-3-methylimidazolidine-2-one,
878 N-cyclopropyl-6-(3-((methyl(pyridin-2-yl)amino)methyl)phenyl)quinazoline-4-amine,
879 N-acetyl-N-(3-(4-(cyclopropylamino)quinazolin-6-yl)benzyl)acetamide,
880 1-(3-(4-(pyrrolidin-1-yl)quinazolin-6-yl)benzyl)pyrrolidine-2,5-dione,
881 1-(3-(8-chloro-4-(cyclopropylamino)quinazolin-6-yl)benzyl)pyrrolidine-2,5-dione,
882 1-(3-(4-(cyclopropylamino)-7-fluoroquinazolin-6-yl)benzyl)pyrrolidine-2,5-dione,
883 1-(3-(4-(cyclopropylamino)-8-(trifluoromethyl)quinazolin-6-yl)benzyl)pyrrolidine-2,5-dione,
884 1-(3-(4-(cyclopropylamino)-7-methylquinazolin-6-yl)benzyl)pyrrolidine-2,5-dione,
885 1-(3-(4-(cyclopropylamino)quinazolin-6-yl)benzyl)-5-methylpyrrolidine-2-one,
886 N-allyl-N-(3-(4-(cyclopropylamino)quinazolin-6-yl)benzyl)acetamide,
887 N-(3-(4-(cyclopropylamino)quinazolin-6-yl)benzyl)-N-propylacetamide hydrochloride,
888 N-cyclopropyl-6-(3-((cyclopropyl(pyridin-2-yl)amino)methyl)phenyl)quinazoline-4-amine,
889 1-(3-(4-(cyclopropylamino)quinazolin-6-yl)-5-fluorobenzyl)pyrrolidine-2,5-dione,
890 N-cyclopropyl-N-((6-(4-(cyclopropylamino)quinazolin-6-yl)pyridin-2-yl)methyl)acetamide,
891 N-cyclopropyl-6-(3-((cyclopropyl(methyl)amino)methyl)phenyl)quinazoline-4-amine,
892 N-cyclopropyl-N-(3-(4-(cyclopropylamino)quinazolin-6-yl)benzyl)thiazole-2-amine,
893 N-cyclobutyl-N-(3-(4-(cyclopropylamino)quinazolin-6-yl)benzyl)acetamide,
894 N-(3-(4-(cyclopropylamino)quinazolin-6-yl)benzyl)-N-ethylacetamide,
895 1-(3-(4-(cyclopropylamino)quinazolin-6-yl)-4-fluorobenzyl)pyrrolidine-2,5-dione,
896 N-cyclopropyl-N-(3-(4-(cyclopropylamino)quinazolin-6-yl)-4-fluorobenzyl)acetamide,
897 N-cyclopropyl-N-(3-(4-(cyclopropylamino)quinazolin-6-yl)-5-fluorobenzyl)acetamide,
898 methyl cyclopropyl(3-(4-(cyclopropylamino)quinazolin-6-yl)benzyl)carbamate,
899 1-(3-(4-(cyclopropylamino)-7-methoxyquinazolin-6-yl)benzyl)pyrrolidine-2,5-dione,
900 2-cyano-N-cyclopropyl-N-(3-(4-(cyclopropylamino)quinazolin-6-yl)-4-fluorobenzyl)acetamide,
901 1-(3-(4-(cyclopropyl(methyl)amino)quinazolin-6-yl)benzyl)pyrrolidine-2,5-dione,
902 2-cyano-N-cyclopropyl-N-(3-(4-(cyclopropylamino)quinazolin-6-yl)-5-fluorobenzyl)acetamide,
903 N-cyclopropyl-N-((4-(4-(cyclopropylamino)quinazolin-6-yl)thiazol-2-yl)methyl)acetamide,
904 1-(3-(4-(cyclopropylamino)-5-fluoroquinazolin-6-yl)benzyl)pyrrolidine-2,5-dione,
905 2-cyano-N-cyclopropyl-N-(5-(4-(cyclopropylamino)-7-fluoroquinazolin-6-yl)-2-fluorobenzyl)acetamide,
906 2-cyano-N-cyclopropyl-N-(3-(4-(cyclopropylamino)-7-fluoroquinazolin-6-yl)benzyl)acetamide,
907 1-(3-(4-(cyclopropylamino)-8-fluoroquinazolin-6-yl)benzyl)pyrrolidine-2,5-dione,
908 N-cyclopropyl-6-(3-((cyclopropyl(phenyl)amino)methyl)phenyl)quinazoline-4-amine,
909 1-((4-(4-(cyclopropylamino)quinazolin-6-yl)thiophen-2-yl)methyl)pyrrolidine-2,5-dione,
910 1-(3-(4-(cyclopropylamino)-5,7-difluoroquinazolin-6-yl)benzyl)pyrrolidine-2,5-dione,
911 N-cyclopropyl-N-(3-(4-(cyclopropylamino)quinazolin-6-yl)-2-methylbenzyl)acetamide, and
912 N-cyclopropyl-N-(5-(4-(cyclopropylmethyl)quinazolin-6-yl)-2-methylbenzyl)acetamide,
or a salt thereof.

26. A method for producing a compound corresponding to formula I as claimed in claim 1, said method comprising reacting a compound corresponding to formula II 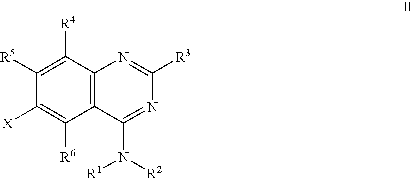
wherein R¹, R², R³, R⁴, R⁵ and R⁶ have the meanings given in claim 1, and X represents a leaving group, preferably for a halogen residue or a sulfonic ester,
with a compound corresponding to formula III 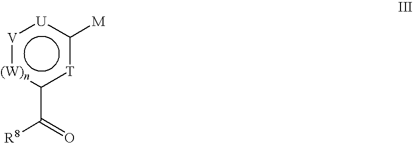
wherein T, U, V, W, n and R⁸ have the meanings given in claim 1, and
M represents —MgY or —ZnY, wherein Y represents a halogen residue or a sulfonic ester, or
M represents —BF₃K, —B(OH)₂ or —B(OR^{A})₂, wherein R^{A} represents alkyl or two residues R^{A} together with the —O—B—O— group linking them form a heterocycloalkyl residue,
in the presence of a catalyst which may be polymer-bonded, optionally in a reaction medium, optionally in the presence of a base, optionally in the presence of a ligand which may be polymer-bonded, at a temperature of −70° C. to 300° C.,
to yield a corresponding compound corresponding to formula IV 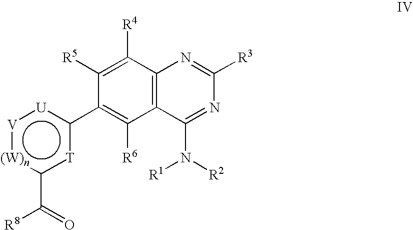
wherein R¹, R², R³, R⁴, R⁵, R⁶, R⁸, T, U, V, W and n have the meanings given above, and optionally purifying or isolating the compound of formula IV; and
reacting the compound corresponding to formula IV in a reaction medium, in the presence of a reducing agent which may be polymer-bonded, at a temperature or −100° C. to 200° C., to yield a compound corresponding to formula V 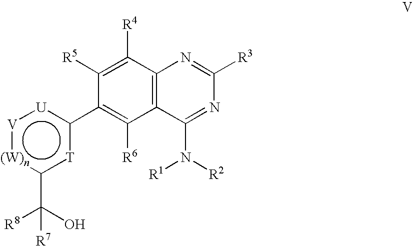
wherein R¹, R², R³, R⁴, R⁵, R⁶, R⁸, T, U, V, W and n have the meanings given above and R⁷ represents H, and optionally purifying or isolating the compound of formula V; and
reacting the compound corresponding to formula V with a compound corresponding to the formula HNR⁹R¹⁰, wherein R⁹ and R¹⁰ have the meanings given in claim 1, in the presence of a compound corresponding to the formula R^{B}—O—C(═O)—N═N—C(═O)—O—R^{B} which may be polymer-bonded, wherein R^{B} represents alkyl or benzyl, and in the presence of a tertiary phosphine which may be polymer-bonded, optionally in a reaction medium, at a temperature of −100° C. to 200° C.,
to yield a compound corresponding to formula I 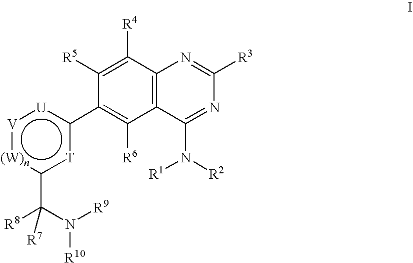
wherein R¹, R², R³, R⁴, R⁵, R⁶, R⁸, R⁹, R¹⁰, T, U, V, W and n have the meanings given above and R⁷ represents H, and optionally purifying or isolating the compound of formula I;
or reacting a compound corresponding to formula IV with a compound corresponding to the formula H₂NR⁹, wherein R⁹ is as specified above,
in a reaction medium, in the presence of a reducing agent which may be polymer-bonded, or in the presence of a catalyst in a hydrogen atmosphere, at a temperature of −100° C. to 200° C., to yield a compound corresponding to formula I, wherein R¹, R², R³, R⁴, R⁵, R⁶, R⁸, R⁹, T, U, V, W and n have the meanings given above, and R⁷ and R¹⁰ respectively represent H, and optionally purifying or isolating the compound of formula I; and
optionally reacting compound corresponding to formula I, wherein R¹, R², R³, R⁴, R⁵, R⁶, R⁸, R⁹, T, U, V, W and n have the meanings given above, and R⁷ and R¹⁰ each represent H, with a compound corresponding to the formula Z-S(═O)₂—R²¹, wherein R²¹ has the meaning given in claim 1, and Z represents a leaving group, optionally in a reaction medium, optionally in the presence of a base, at a temperature of −70° C. to 200° C.,
to yield a compound corresponding to formula I, wherein R¹, R², R³, R⁴, R⁵, R⁶, R⁸, R⁹, T, U, V, W and n have the meanings given above, R⁷ represents H, and R¹⁰ represents —S(═O)₂—R²¹, and optionally purifying or isolating the compound of formula I; or
optionally reacting a compound corresponding to formula I, wherein R¹, R², R³, R⁴, R⁵, R⁶, R⁸, R⁹, T, U, V, W and n have the meanings given above, and R⁷ and R¹⁰ each represent H, with a compound corresponding to formula Z-C(═O)—R¹⁵, wherein R¹⁵ has the meaning given in claim 1, and Z represents a leaving group, optionally in a reaction medium, optionally in the presence of a base, at a temperature of −70° C. to 200° C.,
to yield a compound corresponding to formula I, wherein R¹, R², R³, R⁴, R⁵, R⁶, R⁸, R⁹, T, U, V, W and n have the meanings given above, R⁷ represents H, and R¹⁰ represents —C(═O)—R¹⁵, and optionally purifying or isolating the compound of formula I; or
optionally reacting a compound corresponding to formula I, wherein R¹, R², R³, R⁴, R⁵, R⁶, R⁸, R⁹, T, U, V, W and n have the meanings given above and R⁷ and R¹⁰ respectively represent H,
with a compound corresponding to the formula OH—C(═O)—R¹⁵, wherein R¹⁵ has the meaning given in claim 1, in the presence of a coupling reagent which may be polymer-bonded, at a temperature of −70° C. to 200° C., optionally in a reaction medium, optionally in the presence of a base,
to yield a compound corresponding to formula I, wherein R¹, R², R³, R⁴, R⁵, R⁶, R⁸, R⁹, T, U, V, W and n have the meanings given above, R⁷ represents H, and R¹⁰ represents —C(═O)—R¹⁵; and optionally purifying or isolating the compound of formula I; or
optionally reacting a compound corresponding to formula I, wherein R¹, R², R³, R⁴, R⁵, R⁶, R⁸, R⁹, T, U, V, W and n have the meanings given above, and R⁷ and R¹⁰ each represent H, with a compound corresponding to formula R¹⁷—N═C═O or a compound corresponding to formula R¹⁷—N═C═S, wherein R¹⁷ has the meaning given in claim 1, optionally in a reaction medium, optionally in the presence of a base, at a temperature of −70° C. to 200° C.,
to yield a compound corresponding to formula I, wherein R¹, R², R³, R⁴, R⁵, R⁶, R⁸, R⁹, T, U, V, W and n have the meanings given above; R⁷ represents H, and R¹⁰ represents —C(═O)—NH—R¹⁷ or —C(═S)—NH—R¹⁷, and optionally purifying or isolating the compound of formula I.

27. A method for producing a compound corresponding to formula I as claimed in claim 1, said method comprising reacting a compound corresponding to formula II 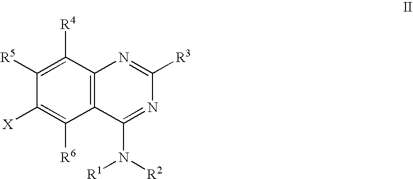
wherein R¹, R², R³, R⁴, R⁵ and R⁶ have the meanings given in claim 1, and X represents a leaving group,
with a compound corresponding to formula VI 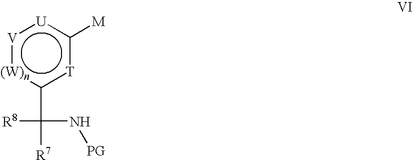
wherein T, U, V, W, n, R⁷ and R⁸ have the meanings given in claim 1, M represents —BF₃K, —B(OH)₂ or —B(OR^{A})₂, wherein R^{A} represents alkyl or two residues R^{A} together with the —O—B—O— group linking them form a heterocycloalkyl group, and PG denotes a protecting group, in the presence of a catalyst which may be polymer-bonded, optionally in a reaction medium, optionally in the presence of a base, optionally in the presence of a ligand which may be polymer-bonded, at a temperature of −70° C. to 300° C.,
to yield a compound corresponding to formula VII 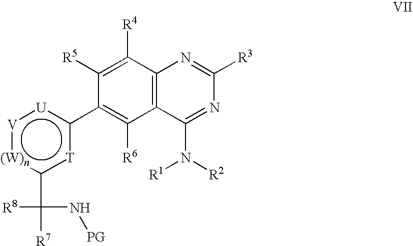
wherein R¹, R², R³, R⁴, R⁵, R⁶, R⁷, R⁸, T, U, V, W, n and PG have the meanings given above, and optionally purifying or isolating the compound of formula VII; and
if PG represents a tert-butyloxycarbonyl or 9-fluoroenylmethyloxy carbonyl group, optionally reacting the compound corresponding to formula VII in a reaction medium, in the presence of a acid, at a temperature of between −70° C. to 100° C., or
if PG represents a benzyl group or benzyloxycarbonyl group, optionally reacting the compound corresponding to formula VII in a reaction medium, in the presence of hydrogen and in the presence of a catalyst, at a temperature of between −70° C. to 200° C.,
to yield a compound corresponding to formula I or salt thereof 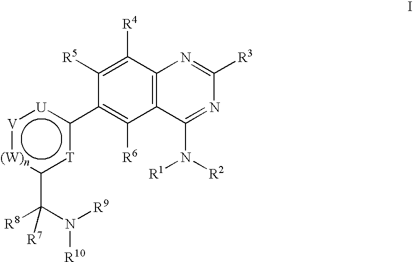
wherein R¹, R², R³, R⁴, R⁵, R⁶, R⁷, R⁸, R¹⁰, T, U, V, W and n have the meanings given above, and R⁹ and R¹⁰ each represent H, and optionally purifying or isolating the compound of formula I; and
optionally reacting a compound corresponding to formula I, wherein R¹, R², R³, R⁴, R⁵, R⁶, R⁷, R⁸, T, U, V, W and n have the meanings given above, and R⁹ and R¹⁰ each represent H, with a compound corresponding to formula Z-S(═O)₂—R²¹, wherein R²¹ has the meaning given in claim 1, and Z represents a leaving group, optionally in a reaction medium, optionally in the presence of a base, at a temperature of −70° C. to 200° C.,
to yield a compound corresponding to formula I, wherein R¹, R², R³, R⁴, R⁵, R⁶, R⁷, R⁸, R⁹, T, U, V, W and n have the meanings given above and R¹⁰ represents —S(═O)₂—R²¹, and optionally purifying or isolating the compound of formula I; or
optionally reacting a compound corresponding to formula I, wherein R¹, R², R³, R⁴, R⁵, R⁶, R⁷, R⁸, T, U, V, W and n have the meanings given above, and R⁹ and R¹⁰ each represent H, with a compound corresponding to formula Z-C(═O)—R¹⁵, wherein R¹⁵ has the meaning given in claim 1, and Z represents a leaving group, optionally in a reaction medium, optionally in the presence of a base, at a temperature of −70° C. to 200° C.,
to yield a compound corresponding to formula I, wherein R¹, R², R³, R⁴, R⁵, R⁶, R⁷, R⁸, R⁹, T, U, V, W and n have the meanings given above, and R¹⁰ represents —C(═O)—R¹⁵, and optionally purifying or isolating the compound of formula I; or
optionally reacting a compound corresponding to formula I, wherein R¹, R², R³, R⁴, R⁵, R⁶, R⁷, R⁸, T, U, V, W and n have the meanings given above, and R⁹ and R¹⁰ each represent H, with a compound corresponding to the formula OH—C(═O)—R¹⁵, wherein R¹⁵ has the meaning given in claim 1, in the presence of a coupling reagent which may be polymer-bonded, optionally in a reaction medium, optionally in the presence of a base, at a temperature of −70° C. to 200° C.,
to yield a compound corresponding to formula I, wherein R¹, R², R³, R⁴, R⁵, R⁶, R⁷, R⁸, R⁹, T, U, V, W and n have the meanings given above and R¹⁰ represents —C(═O)—R¹⁵, and optionally purifying or isolating the compound of formula I; or
optionally reacting a compound corresponding to formula I, wherein R¹, R², R³, R⁴, R⁵, R⁶, R⁷, R⁸, T, U, V, W and n have the meanings given above, and R⁹ and R¹⁰ each represent H, with a compound corresponding to the formula R¹⁷—N═C═O or a compound corresponding to the formula R¹⁷—N═C═S, wherein R¹⁷ has the meaning given in claim 1, optionally in a reaction medium, optionally in the presence of a base, at a temperature of −70° C. to 200° C., to yield a compound corresponding to formula I, wherein R¹, R², R³, R⁴, R⁵, R⁶, R⁷, R⁸, R⁹, T, U, V, W and n have the meanings given above and R¹⁰ represents —C(═O)—NH—R¹⁷ or —C(═S)—NH—R¹⁷, and optionally purifying or isolating the compound of formula I; or
optionally reacting a compound corresponding to formula I, wherein R¹, R², R³, R⁴, R⁵, R⁶, R⁷, R⁸, T, U, V, W and n have the meanings given above, and R⁹ and R¹⁰ each represent H, with a compound having at least two substituents independently selected from the group consisting of bromine, chlorine, —S(═O)₂—Cl, —S(═O)₂—Br, —C(═O)—Cl and —C(═O)—Br, optionally in a reaction medium, optionally in the presence of a base, at a temperature of −70° C. to 200° C.,
to yield a compound corresponding to formula I, wherein R¹, R², R³, R⁴, R⁵, R⁶, R⁷, R⁸, T, U, V, W and n have the meanings given above, and R⁹ and R¹⁰ together with the nitrogen atom to which they are bound form a heterocycloalkyl or a heterocycloalkenyl residue as defined in claim 1, and optionally purifying or isolating the compound of formula I.

28. A method for producing a compound corresponding to formula I as claimed in claim 1, said method comprising reacting a compound corresponding to formula II 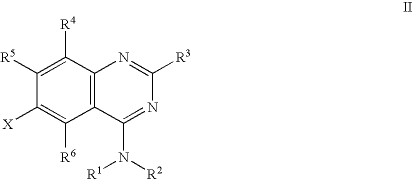
wherein R¹, R², R³, R⁴, R⁵ and R⁶ have the meanings given in claim 1, and X represents a leaving group,
with a compound corresponding to formula III 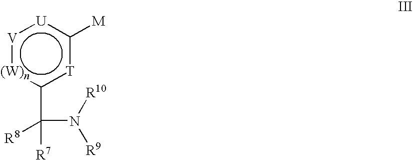
wherein T, U, V, W, n, R⁷, R⁸, R⁹ and R¹⁰ have the meanings given in claim 1, and
M represents —MgY or —ZnY, wherein Y represents a halogen residue or a sulfonic ester, or
M represents —BF₃K, —B(OH)₂ or —B(OR^{A})₂, wherein R^{A} represents alkyl or two residues R^{A} together with the —O—B—O— group linking them form a heterocycloalkyl residue, in the presence of a catalyst, which may be polymer-bonded, optionally in a reaction medium, optionally in the presence of a base, optionally in the presence of a ligand, which may be polymer-bonded, at a temperature of −70° C. to 300° C.,
to yield a compound corresponding to formula I 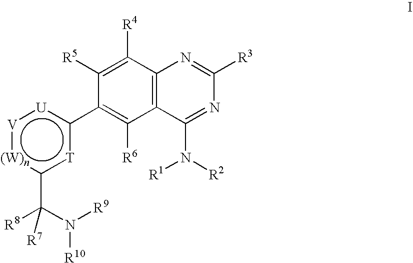
wherein R¹, R², R³, R⁴, R⁵, R⁶, R⁷, R⁸, R⁹, R¹⁰, T, U, V, W and n have the meanings given above, and optionally purifying or isolating the compound of formula I.

29. A pharmaceutical composition comprising a compound as claimed in claim 1 and at least one physiologically acceptable adjuvant.

30. A method of regulating mGluR5 receptors in a subject, said method comprising administering to said subject an effective mGluR5 receptor regulating amount of a compound as claimed in claim 1.

31. A method of treating or inhibiting a disorder or disease state at least partially mediated by mGluR5 receptors in a subject, said method comprising administering to said subject a pharmacologically effective amount of a compound as claimed in claim 1.

32. A method as claimed in claim 31, wherein said disorder or disease state is selected from the group consisting of pain; migraine; depression; neurodegenerative diseases; cognitive diseases; psychiatric disorders; epilepsy; coughing; urinary incontinence; diarrhea; pruritus; schizophrenia; cerebral ischaemia; muscle spasms; cramps; lung diseases; vomiting; stroke; dyskinesia; retinopathy; lethargy; laryngitis; eating disorders; alcohol dependency or abuse; drug or pharmaceutical dependency or abuse; withdrawal symptoms from dependency on alcohol, drugs or pharmaceuticals; development of tolerance to medications; gastro-oesophageal reflux syndrome; gastro-oesophageal reflux disease; irritable bowel syndrome; or for diuresis; for antinatriuresis; for influencing the cardiovascular system; for increasing vigilance; for increasing libido; for modulating movement activity or for local anaesthesia.

33. A method as claimed in claim 32, wherein said disorder or disease state is pain selected from the group consisting of acute pain, chronic pain, neuropathic pain and visceral pain; a neurodegenerative disease selected from the group consisting of multiple sclerosis, Alzheimer's disease, Parkinson's disease and Huntington's disease; cognitive deficiencies; a psychiatric disorder selected from the group consisting of anxiety, and panic attacks; a lung disease selected from the group consisting of asthma, and pseudo-croup; an eating disorder selected from the group consisting of bulimia, cachexia, anorexia and obesity; nicotine or cocaine dependency or abuse; or development of tolerance to natural or synthetic opioids.

34. A method as claimed in claim 31, wherein said disorder or disease state is pain selected from the group consisting of acute pain, chronic pain, neuropathic pain and visceral pain; a psychiatric disorder selected from the group consisting of anxiety and panic attacks; alcohol dependency; attention deficit disorder; gastro-oesophageal reflux, or irritable bowel syndrome.
